(19) United States
(12) Patent Application Publication
(10) Pub. No.: US 2007/0032502 A1
(43) Pub. Date: Feb. 8, 2007

(54) Quinazoline derivatives useful in cancer treatment

(75) Inventors: Alan K. Mallams, Hackettstown, NJ (US); Bimalendu Dasmahapatra, Nutley, NJ (US); Bernard R. Neustadt, West Orange, NJ (US); Mark Demma, Edison, NJ (US); Henry A. Vaccaro, South Plainfield, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/486,358

(22) Filed: Jul. 13, 2006

Related U.S. Application Data
(60) Provisional application No. 60/700,058, filed on Jul. 15, 2005.

(51) Int. Cl. A61K 31/517; C07D 403/02

(52) U.S. Cl. 514/252.17; 544/284

(57) ABSTRACT

The present invention provides compounds of Formula I (wherein X, m, R¹, R², R³, and R⁴ are as defined herein). 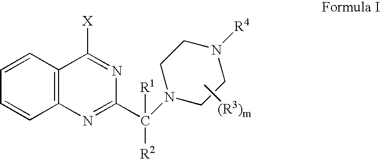 or a pharmaceutically acceptable salt, solvate or ester thereof. The present invention also provides compositions comprising these compounds that are useful for treating cellular proliferative diseases, disorders associated with activity of mutants of p53, or in causing apoptosis of cancer cells.

This Application claims the benefit of U.S. Provisional Application Ser. No. 60/700,058, filed Jul. 15, 2005, which is incorporated herein by reference in its entirety.

### FIELD OF THE INVENTION

The present invention relates to compounds and compositions that are useful for treating cellular proliferative diseases, disorders associated with mutants of p53 activity, or in causing apoptosis of cancer cells. The compounds of the present invention are capable of restoring the biochemical and biological activity of mutant p53 and in causing apoptosis of cancer cells.

### BACKGROUND OF THE INVENTION

Cancer is a leading cause of death in the United States and throughout the world. Cancer cells are often characterized by constitutive proliferative signals, defects in cell cycle checkpoints, as well as defects in apoptotic pathways. There is a great need for the development of new chemotherapeutic drugs that can block cell proliferation and enhance apoptosis of tumor cells.

The p53 tumor suppressor protein belongs to a superfamily of transcription factors that includes its homologs p63 and p73. p53 is involved in a wide range of cellular activities that help ensure the stability of the genome, whereas p63 and p73 are involved in ectodermal morphogenesis, limb morphogenesis, neurogenesis, and homeostatic control and are not considered tumor suppressor genes (1). p53 is involved in DNA damage repair, cell cycle arrest, and apoptosis via transcriptional regulation of genes involved in these activities or by direct interaction with other proteins (2-4). Mutations that inactivate p53 are present in over 50% of all cancers and are indicative of aggressive cancers that are difficult to treat by chemotherapy or ionizing radiation (2, 5).

The majority of inactivating mutations reside in the central core DNA binding domain (DBD) of p53 (2, 5). These mutations can be divided into two main classes, DNA contact mutants, like R273H, where the mutation alters a residue involved in contact with DNA, and structural mutants, like R249S, which result in structural changes in the p53 core domain (6-8). These mutations affect the function of p53 by distorting the structure and reducing the thermal stability of the protein (6-8). This can alter the ability of p53 to bind to various p53 response elements in a variety of genes, hampering its transcriptional regulation (9). In addition, these mutations may alter p53 structure, so that p53 can no longer induce apoptosis by binding to BclXL, thereby inhibiting its anti-apoptotic function (10).

One potential therapeutic approach to cancer would be restoration of growth suppression activity to mutant p53. Several approaches have been tried, ranging from micro-injection of monoclonal antibody 421, C-terminal peptide of p53 and small molecules (11-16). Recently, small molecules and peptides, such as CP-31398, PRIMA1, and CDB3 peptide, have been shown to be effective in restoring p53 function (17-25). Both PRIMAL and CDB3 have been shown to restore p53 DNA-binding activity in vitro (18-21), whereas the effects for CP-31398 have been shown primarily in cell-based assays (17, 22-25). Both CP-31398 and PRIMA1 have been shown to reduce tumor size in animal models (17, 18). It is postulated that the two molecules perform similar tasks, but by different mechanisms. PRIMAL has been suggested to work more broadly to restore p53 DNA-binding activity, but the specific mechanism is not known (18). CP-31398, on the other hand, has been suggested to stabilize p53 as a protectant against thermal denaturation and maintain monoclonal antibody 1620 epitope conformation in newly synthesized p53 (17). Recently, CP-31398 has also been shown to stabilize wild type p53 in cells by inhibiting Mdm2-mediated ubiquitination and degradation (23). Reports from other studies suggest that CP-31398 interacts with DNA and not with p53 in vitro, and it is proposed to act as a DNA-damaging agent (26).

As indicated above, the p53 tumor suppressor protein is mutated in many human cancers and tumerogenicity can be inhibited by reintroduction of the wild type gene. Most of these mutations, which map to the central DBD, appear to cause conformational changes in the domain with loss of DNA binding and sequence specific transcriptional regulatory functions. Therefore, restoring transcriptional regulatory function to mutant p53 represents an attractive target to develop novel chemotherapeutics.

International patent publication WO 2004111014 (published Jun. 4, 2004) refers to quinazolines as modulators of ATP-binding cassette transporters.

Hori et al. (Chemical and Pharmaceutical Bulletin (1993), 41 (6), 1114-17)) disclose the preparation of certain piperazinomethyl quinazolines.

### REFERENCES

1. Bernard, J., Douc-Rasy, S., and Ahomadegbe, J.-C. (2003) Human Mutat. 2, 182-191.
2. Lane, D. P., and Hupp, T. R. (2003) Drug Discov. Today 8, 347-355.
3. Vousden, K. (2000) Cell 103, 691-694.
4. Willis, A. C., and Chen, X. (2002) Curr. Mol. Med. 2, 329-345.
5. Bullock, A. N., and Fersht, A. R. (2001) Nat Rev. Cancer 1, 68-76.
6. Wong, K.-B., DeDecker, B. S., Freund, S. V., Proctor, M. R., Bycroft, M., and Fersht, A. R. (1999) Proc. Nat. Acad. Sci U.S. A. 96, 8348-8442.
7. Bullock, A. R., Henckel, J., DeDecker, B. S., Johnson, C. M., Nikolova, P. V., Proctor, M. R., Lane, D. P., and Fersht, A. R. (1997) Proc. Nat. Acad. Sci. U.S.A. 94, 14338-14343.
8. Bullock, A. N., Henckel, J., and Fersht, A. R. (2000) Oncogene 19, 1245-1256.
9. Nicholls, C. D., McLure, K. G., Shields, M. A., and Lee, P. W. K. (2002) J. Biol. Chem. 277, 12937-12945.
10. Mihara, M., Erster, S., Zaika, A., Petrenko, O., Chittenden, T., Pancoska, P., and Moll, U. M. (2003) Mol. Cell 11, 577-590.
11. Abarzua, P., LoSardo, J. E., Gubler, M. L., Spathis, R., Lu, Y.-A., Felix, A., and Neri, A. (1996) Oncogene 13, 2477-2482.
12. Abarzua, P., LoSardo, J. E., Gubler, M. L., and Neri, A. (1995) Cancer Res. 55, 3490-3494.
13. Halazonetis, T. D., Davis, L. J., and Kandil, A. N. (1993) EMBO J. 12, 1021-1028.
14. Wieczorek, A. M., Waterman, J. L. F., Waterman, J. F., and Halazonetis, T. D. (1996) Nat. Med. 2, 1143-1146.
15. Selinova, G., Ryabchenko, L., Jansson, E., lotsova, V., and Wiman, K. G. (1999) Mol. Cell Biol. 19, 3395-3402.
16. Peng, Y., Li, C., Sebti, S., and Chen, J. (2003) Oncogene 22, 4478-4487.
17. Foster, B. A., Coffey, H. A., Morin, M. J., and Rastinejad, F. (1999) Science 286, 2507-2510.
18. Bykov, V. J. N., Issaeva, N., Shilov, A., Hultcrantz, M., Pugacheva, E., Chumakov, P., Bergman, J., Wiman, K. G., and Selinova, G. (2002) Nat. Med. 8, 282-288.
19. Friedler, A., Hansson, L. O., Veprintsev, D. B., Freund, S. M. V., Rippin, T. M., Nikolova, P. V., Proctor, M. R., Rudiger, S., and Fersht, A. R. (2002) Proc. Nat. Acad. Sci. U.S.A. 99, 937-942.
20. Freidler, A., Verprintsev, D. B., Hansson, L., and Fersht, A. R. (2003) J. Biol. Chem. 278, 24108-24112.
21. Issaeva, N., Friedler, A., Bozko, P., Wiman, K. G., Fersht, A. R., and Selivanova, G. (2003) Proc. Nat. Acad. Sci. U.S.A. 100, 13303-13307.
22. Luu, Y., Bush, J., Cheung, K.-J., Jr., and Li, G. (2002) Exp. Cell Res. 276, 214-222.
23. Wang, W., Takimoto, R., Ratinejad, F., and El-Diery, W. (2003) Mol. Cell Biol. 23, 2171-2181.
24. Wischhusen, J., Naumann, U., Ohgaki, H., Rastinejad, F., and Weller, M. (2003) Oncogene 22, 8233-8245.
25. Takimoto, R., Wang, W., Dicker, D. T., Rastinejad, F., Lyssikatos, J., and El-Diery, W. S. (2002) Cancer Biol. Ther. 1, 47-55.
26. Rippin, T. M., Bykov, V. J. N., Freund, S. M. V., Selivanova, G., Wiman, K. G., and Fersht, A. R. (2002) Oncogene 21, 2119-2129

### SUMMARY OF THE INVENTION

The compounds of the present invention which include 4-(substituted hydroxy)- and 4-(substituted amino)-2-(substituted piperazinyl)quinazolines, are anticancer agents that are capable of restoring the biochemical and biological activity of mutant p53 and in causing apoptosis of cancer cells.

In one embodiment, the present invention provides a compound represented by the structural Formula I: 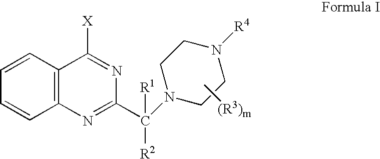
or a pharmaceutically acceptable salt, solvate or ester thereof, wherein:

(i) m is 0 to 2;

(ii) X is OR⁵ or N(R⁶)₂;

(iii) R¹ and R² are each independently selected from the group consisting of hydrogen and alkyl;

(iv) each R³ independently is alkyl;

(v) R⁴ is selected from the group of substituents consisting of alkyl, alkenyl, alkynyl, cycloalkyl, cycloalkenyl, aryl, heteroaryl, heterocyclyl, R⁷—(C═O)—, R⁸—(S(O)₂)—, and —(C═O)—NR⁹R¹⁰—, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)-2-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when the aforesaid cycloalkyl and aryl substituents contains two moieties on the same carbon, such moieties may optionally be taken together with the carbon atom to which they are attached to form a carbocyclic or heterocyclic ring; wherein each of the aforesaid moieties containing an aryl alternative may optionally be independently substituted by one or two radicals independently selected from the group consisting of alkyl, halo, alkoxy, cyano, perhaloalkyl and perhaloalkoxy;

wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—;

wherein when said aryl moiety contains two radicals on adjacent carbon atoms anywhere within said moiety, such radicals may optionally and independently in each occurrence, be taken together with the carbon atoms to which they are attached to form a five to six membered carbocyclic or heterocyclyl ring; wherein each of the aforementioned radicals containing an aryl alternative may optionally be independently substituted by one or two radicals independently selected from the group consisting of alkyl, halo, alkoxy, cyano, perhaloalkyl and perhaloalkoxy;

(vi) R⁵, and each R⁶ are independently selected from the group consisting of hydrogen, alkyl, alkenyl, alkynyl, cycloalkyl, cycloalkenyl, aryl, heteroaryl, and heterocyclyl, wherein each of the R⁵ and R⁶ substituents alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, H₂N—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—NH—(C═O)—, alkyl-O—NH—(C═O)-alkyl-NH—(C═O)—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said cycloalkyl or aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring, which carbocyclic or heterocyclic ring may optionally be fused to an aryl ring;

wherein each of said aryl, cycloalkyl, heterocyclyl and heteroaryl moieties of said R⁵ and R⁶ substituents may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-—N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy;

wherein when X is N(R⁶)₂, the two R⁶ groups may optionally be taken together with the nitrogen atom to which they are shown attached to form a heterocyclyl or heteroaryl ring which heterocyclyl or heteroaryl ring may optionally be independently substituted with one to two substituents independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;

wherein each of the aforesaid alkyl, alkenyl, aryl, arylalkyl-, cycloalkyl, heteroaryl, and heterocyclyl substituents may optionally be independently substituted with one to two moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)-(alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl, heterocyclycl, heteroaryl and aryl moieties contains two radicals on adjacent carbon atoms, such radicals may optionally and independently in each occurrence, be taken together with the carbon atoms to which they are attached to form a five to six membered carbocyclic or heterocyclic ring; wherein when each of said cycloalkyl, heterocyclycl, heteroaryl and aryl moieties contains two radicals on the same carbon, such moieties may optionally be taken together with the carbon atom to which they are attached to form a five to six membered carbocyclic or heterocyclic ring; wherein each of the aforesaid moieties containing an aryl alternative may optionally be substituted by one or two radicals independently selected from the group consisting of alkyl, halo, alkoxy, cyano, perhaloalkyl and perhaloalkoxy;

(vii) R⁷ is selected from the group of substitutents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O), aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said R⁷ aryl or cycloalkyl substituent contains two moieties on adjacent carbon atoms, such moieties may optionally and independently in each occurrence, be taken together with the carbon atoms to which they are attached to form a five to six membered carbocyclic or heterocyclyl ring; wherein each of said moieties containing an aryl alternative may optionally be substituted by one or two radicals independently selected from the group consisting of alkyl, halo, alkoxy, cyano, perhaloalkyl and perhaloalkoxy;

(viii) R⁸ is selected from the group of substituents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said R⁸ aryl or cycloalkyl substituent contains two moieties on adjacent carbon atoms, such moieties may optionally and independently in each occurrence, be taken together with the carbon atoms to which they are attached to form a five to six membered carbocyclic or heterocyclyl ring, which carbocyclic or heterorcycli ring may optionally be substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy;

(ix) R⁹ is selected from the group of substituents consisting of alkyl, alkenyl, alkynyl, cycloalkyl, cycloalkenyl, aryl, heteroaryl, and heterocyclyl, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;

wherein each of said aryl, cycloalkyl, heterocyclyl, and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said radicals containing an aryl alternative may optionally be substituted by one or two groups independently selected from the group consisting of alkyl, halo, alkoxy, cyano, perhaloalkyl and perhaloalkoxy; and

(x) R¹⁰ is selected from the group consisting of hydrogen and alkyl;

with the following provisos:

(a) when X is OR⁵, R⁴ and R⁵ simultaneously are other than unsubstituted alkyl;

(b) when X is OR⁵, R⁴ is other than R⁸—(S(O)₂)—;

(c) when X is N(R⁶)₂ wherein each R⁶ is independently hydrogen or straight or branched alkyl with no further substitution, and R⁴ is R⁸—(S(O)₂)— wherein R⁸ is an aryl which may optionally be substituted, the substituents on said aryl are other than alkoxy and halo; and

(d) when X is N(R⁶)₂ wherein the two R⁶ groups are taken together with the nitrogen atom to which they are shown attached form a piperidine ring, R⁴ is other than R⁸—S(O)₂—.

Pharmaceutical formulations or compositions for the treatment of cellular proliferative diseases, disorders associated with mutant p53 activity, for restoring biological or biochemical acitivity of mutant p53, and/or for causing apoptosis of cancer cells in a subject comprising administering a therapeutically effective amount of at least one of the inventive compounds and a pharmaceutically acceptable carrier to the subject also are provided.

Methods of treating cellular proliferative diseases, disorders associated with mutant p53 activity, for restoring biological or biochemical acitivity of mutant p53, and/or for causing apoptosis of cancer cells in a subject in need of such treatment an effective amount of at least one of the inventive compounds also are provided.

Processes for preparing the compound of formula I are also provided.

Other than in the operating examples, or where otherwise indicated, all numbers expressing quantities of ingredients, reaction conditions, and so forth used in the specification and claims are to be understood as being modified in all instances by the term “about.”

### DESCRIPTION OF DRAWINGS

FIG. 1 is a human DLD-1 tumor growth curve (unstaged).

FIG. 2 is a plot of tumor growth inhibition (unstaged) at various concentration of compound #1 of the invention.

FIG. 3 is a human DLD-1 tumor growth curve (staged).

FIG. 4 is a plot of tumor growth inhibition (staged) at various concentration of compound #1 of the invention.

FIG. 5 is an illustration of an increase in sensitivity of pancreatic cancer cells to temozolamide in the presence of the compound # 25-36 of the invention.

### DETAILED DESCRIPTION

In one embodiment, the present invention discloses compounds represented by structural Formula I or a pharmaceutically acceptable salt or ester thereof, wherein the various moieties are as described above.

In another embodiment, X in above Formula I is N(R⁶)₂:

In another embodiment, in formula I, R¹ and R² are both hydrogen.

In another embodiment, in formula I, m is 0 or 1.

In another embodiment, in formula I, m is 0.

In another embodiment, in formula I, X is N(R⁶)₂, m is 0 or 1, and R⁴ is selected from the group of substituents consisting of alkyl and alkenyl;

wherein said R⁴ alkyl and alkenyl substituents may optionally be independently substituted by one to four moieties selected independently from the group consisting of alkyl-S—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy;

wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—;

wherein when said aryl moiety contains two radicals on adjacent carbon atoms anywhere within said moiety, such radicals may optionally and independently in each occurence be taken together with the carbon atoms to which they are attached to form a five to six membered carbocyclic or heterocyclic ring; wherein each of the aforementioned radicals containing an aryl alternative may optionally be independently substituted by one or two radicals independently selected from the group consisting of alkyl, halo, alkoxy, cyano, perhaloalkyl and perhaloalkoxy.

In another embodiment, in addition to all of the limitations of the preceding embodiment, each of said R⁴ alkyl and alkenyl substituents may optionally be independently substituted by one to four moieties selected independently from the group consisting of aryl, cycloalkyl, heterocyclyl, heteroaryl, alkyl-S—, and fluorenyl;

wherein said aryl moiety may optionally be independently substituted by one to two radicals selected independently from the group consisting of alkyl, alkoxy, halo, hydroxyl, cyano, alkyl-S—, aryl-S-alkyl-S(O)₂—, alkyl-(C═O)—NH—, alkyl-O—(C═O)—, perhaloalkyl, aryl, aryloxy, aryl-alkynyl-, and alkyl-O—(C═O)-alkyl-O—; wherein when said aryl moiety contains two radicals on adjacent carbon atoms anywhere within said moiety, such radicals may optionally and independently in each occurrence, be taken together with the carbon atoms to which they are attached to form a five to six membered carbocyclic or heterocyclic ring; wherein each of said radicals containing an aryl alternative may optionally be substituted by one or two radicals independently selected from the group consisting of alkyl, halo, alkoxy, cyano, perhaloalkyl and perhaloalkoxy;

wherein said cycloalkyl moiety may optionally be independently substituted by one or two radicals selected independently from the group consisting of alkyl, halo, hydroxy, cyano, and alkyl-O—(C═O)—;

wherein said heterocyclyl moiety may optionally be independently substituted by one or two radicals selected independently from the group consting of halo, hydroxyl, alkoxy;

wherein said heteroaryl moiety may optionally be independently substituted by one or two radicals selected independently from the group consting of alkyl, hydroxyalkyl, heteroaryl, aryl, and aryl-S(O)₂—; wherein each of said radicals containing an aryl alternative may optionally be substituted by one or two radicals independently selected from the group consisting of alkyl, halo, alkoxy, cyano, perhaloalkyl and perhaloalkoxy.

In another embodiment, X is N(R⁶)₂, m is 0 or 1, and the cycloalkyl moiety of the R⁴ alkyl and alkenyl substituents is selected from the group consisting of cyclopropyl, cyclobutyl, cyclopentyl, cyclohexyl and cycloheptyl, each of which may be optionally substituted.

In another embodiment, X is N(R⁶)₂, m is 0 or 1, and the heterocyclyl moiety of the R⁴ alkyl and alkenyl substituents is selected from the group consisting of dihydropyranyl, tetrahydropyranyl, and piperidinyl, each of which may be optionally substituted.

In another embodiment, X is N(R⁶)₂, m is 0 or 1, and the heteroaryl moiety of the R⁴ alkyl and alkenyl substituents is selected from the group consisting of pyridinyl, furanyl, thiophenyl, pyrrolyl, 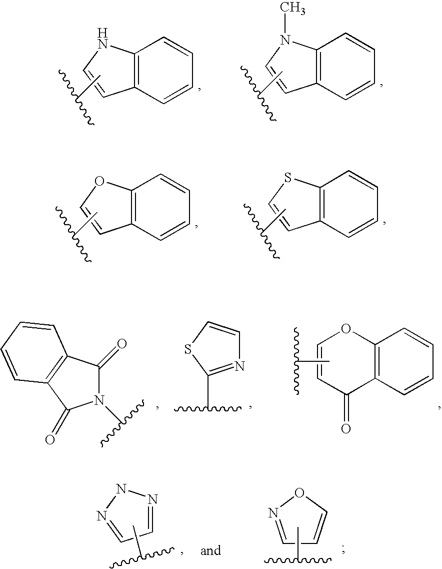
each of which may be optionally substituted.

In another embodiment, X is N(R⁶)₂, m is 0 or 1, and the aryl moiety of the R⁴ alkyl and alkenyl substituents, including aryl moiety containing two radicals on adjacent carbon atoms which are taken together with the carbon atoms to which said radicals are attached to form a five to six memebered carbocyclic or heterocyclic ring, is selected from the group consisting of phenyl, naphthyl, 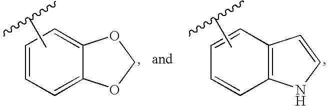
each of which may optionally be substituted.

In another embodiment, X is N(R⁶)₂, m is 0 or 1, and R⁴ is selected from group of substituents consisting of cycloalkyl, cycloalkenyl and heterocyclyl;

wherein each of the aforesaid cycloalkyl, cycloalkenyl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of the aforesaid cycloalkyl, cycloalkenyl and heterocyclyl substituents contains two moieties on the same carbon, such moieties may optionally be taken together with the carbon atom to which they are attached to form a carbocyclic or heterocyclic ring; wherein when each of the aforesaid cycloalkyl, cycloalkenyl and heterocyclyl substituents contain two moieties on adjacent carbon atoms anywhere within said substituent, such moieties may optionally and independently in each occurrence, be taken together with the carbon atoms to which they are attached, to form a five to six membered carbocyclic or heterocyclic ring; wherein said aryl moiety and each of the aforesaid moieties containing an aryl alternative may optionally be independently substituted by one or two radicals independently selected from the group consisting of alkyl, halo, alkoxy, cyano, perhaloalkyl and perhaloalkoxy.

In another embodiment, X is N(R⁶)₂, m is 0 or 1, and the R⁴ cycloalkyl, cycloalkenyl and heterocyclyl substituents may optionally be independently substituted by one to four moieties independently selected from the group consisting of cyano, alkyl, alkyl-(C═O)—, perhaloalkyl, aryl, and aryl-(C═O)—; wherein when each of the aforesaid cycloalkyl, cycloalkenyl and heterocyclyl substituents contains two moieties on the same carbon, such moieties may optionally be taken together with the carbon atom to which they are attached to form a carbocyclic or heterocyclic ring; wherein when each of the aforesaid cycloalkyl, cycloalkenyl and heterocyclyl substituents contains two moieties on adjacent carbon atoms anywhere within said substituent, such moieties may optionally and independently in each occurrence, be taken together with the carbon atoms to which they are attached, to form a five to six membered carbocyclic or heterocyclic ring; wherein the aryl and aryl-(C═O)— moieties may optionally be independently substituted by one or two radicals independently selected from the group consisting of alkyl, halo, alkoxy, cyano, perhaloalkyl and perhaloalkoxy.

In another embodiment, X is N(R⁶)₂, m is 0 or 1, and the R⁴ cycloalkyl substituent, including including cycloalkyl substituent containing two moieties on adjacent carbon atoms which are taken together with the carbon atoms to which said moieties are attached to form a five to six membered carbocyclic or heterocyclic ring, and including cycloalkyl substituent containing two moieties on the same carbon atom which are taken together with the carbon atom to which said moieties are attached to form a five to six membered carbocyclic or heterocyclic ring, is selected from the group consisting of multicyclic ring system, cyclopropyl, cyclobutyl, cyclopenyl, cyclohexyl, cycloheptyl, polycycloalkyl, 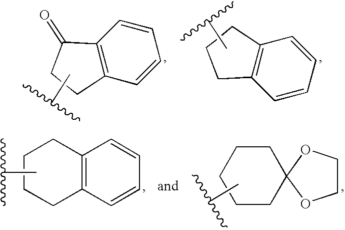
each of which may optionally be substituted.

In another embodiment, X is N(R⁶)₂, m is 0 or 1, and the R⁴ heterocyclyl substituent is selected from the group consisting of tetrahydrofuranyl, tetrahydropyranyl, tetrahydrothiophenyl, tetrahydrothiopyranyl, and piperidinyl, each of which may be optionally substituted.

In another embodiment, X is N(R⁶)₂, m is 0 or 1, and R⁴ is R⁷—(C═O)—; wherein R⁷ is selected from the group of substitutents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-G-(C═O)-[(alkyl)-N]—, alkyl-S—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said said R⁷ aryl, heteroaryl, heterocyclyl, and cycloalkyl substituents contains two moieties on adjacent carbon atoms anywhere within said substituent, such moieties may optionally and independently in each occurrence, be taken together with the carbon atoms to which they are attached to form a five to six membered carbocyclic or heterocyclyl ring; wherein each of said moieties containing an aryl alternative may optionally be substituted by one or two radicals independently selected from the group consisting of alkyl, halo, alkoxy, cyano, perhaloalkyl and perhaloalkoxy.

In another embodiment, X is N(R⁶)₂, m is 0 or 1, and R⁴ is R⁷—(C═O)—, wherein R⁷ is selected from the group of substitutents consisting of alkyl, alkenyl, aryl, cycloalkyl, heteroaryl, and heterocyclyl; wherein when each of said aryl, cycloalkyl, heteroaryl, and heterocyclyl substituents contains two moieties on adjacent carbon atoms anywhere within said substituent, such moieties may optionally and independently in each occurrence, be taken together with the carbon atoms to which such moieties are attached to form a five to six membered carbocyclic or heterocyclic ring;

wherein said alkyl and alkenyl substitutents may optionally be independently substituted with one to four moieties independently selected from the group consisting of alkyl, cycloalkyl, heterocyclyl, alkyl-S—, alkyl-O—(C═O)—, aryl, aryloxy, aryl-S—, and heteroaryl;

wherein said heterocyclyl substituent may optionally be substituted with one to four moieties independently selected from the group consisting of alkyl, halo, alkoxy, and alkyl-(C═O)—,

wherein said heteroaryl substitutent may optionally be substituted with one to four moieties independently selected from the group consisting of alkyl, aryl, halo and alkoxy;

wherein said aryl substitutent may optionally be substituted with one to four moieties independently selected from the group consisting of alkyl, alkyl-S—, cycloalkyl, alkoxy, halo, aryl, cyano, alkyl-(C═O)—NH—, and perhaloalkyl;

wherein said cycloalkyl substituent may optionally be substituted with one to four moieties independently selected from the group consisting of alkyl, halo, alkoxy, and aryl;

wherein said aryl moiety may optionally be substituted with one or two radicals selected from the group consisting of alkyl, cyano, halo, aryl, and perhaloalkyl;

wherein each of the aforesaid moieties containing an aryl alternative may optionally be substituted by one or two radicals independently selected from the group consisting of alkyl, halo, alkoxy, cyano, perhaloalkyl and perhaloalkoxy.

In another embodiment, X is N(R⁶)₂, m is 0 or 1, and R⁴ is R⁷—(C═O)— as set forth above, wherein said R⁷ cycloalkyl substituent, including R⁷ cycloalkyl substituent containing two moieties on adjacent carbon atoms which are taken together with the carbon atoms to which said moieties are attached to form a five to six membered carbocyclic or heterocyclic ring, is selected from the group consisting of multicyclic ring system, cyclopropyl, cyclobutyl, cyclopenyl, cyclohexyl, cycloheptyl, polycycloalkyl, 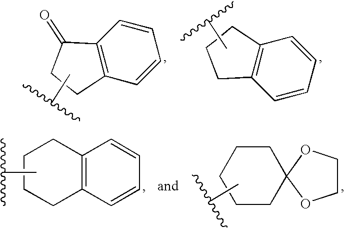
each of which may optionally be substituted.

In another embodiment, X is N(R⁶)₂, m is 0 or 1, and R⁴ is R⁷—(C═O)—, as set forth above, wherein said R⁷ heteroaryl substituent, including R⁷ heteroaryl substituent containing two moieties on adjacent carbon atoms which are taken together with the carbon atoms to which said moieties are attached to form a five to six membered carbocyclic or heterocyclic ring, is selected from the group consisting of pyridinyl, furanyl, thiophenyl, pyrrolyl, 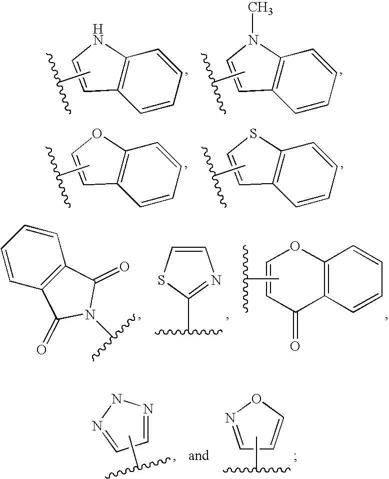
each of which may be optionally substituted.

In another embodiment, X is N(R⁶)₂, m is 0 or 1, and R⁴ is R⁷—(C═O)—, as set forth above, wherein said R⁷ heterocyclyl substituent, including heterocyclyl substituent containing two moieties on adjacent carbon atoms which are taken together with the carbon atoms to which said moieties are attached to form a five to six membered carbocyclic or heterocyclic ring, is selected from the group consisting of tetrahydrofuranyl, tetrahydropyranyl, tetrahydrothiophenyl, tetrahydrothiopyranyl, piperidinyl, 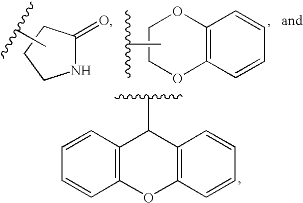
each of which may be optionally substituted.

In another embodiment, X is N(R⁶)₂, m is 0 or 1, and R⁴ is R⁷—(C═O)—, as set forth above, wherein said R⁷ aryl substituent, including aryl substituent containing two moieties on adjacent carbon atoms which are taken together with the carbon atoms to which said moieties are attached, to form a five to six membered carbocyclic or heterocyclic ring, is selected from the group consisting of phenyl, naphthyl, 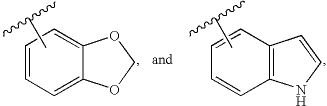
each of which may optionally be substituted.

In another embodiment, X is N(R⁶)₂, m is 0 or 1, and R⁴ is R⁸—(S(O)₂)— wherein R⁸ is selected from the group of substituents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said R⁸ aryl, heteroaryl, heterocyclyl, or cycloalkyl substituents contains two moieties on adjacent carbon atoms anywhere within said substituent, such moieties may optionally and independently in each occurrence, be taken together with the carbon atoms to which they are attached to form a five to six membered carbocyclic or heterocyclic ring, which carbocyclic or heterorcyclic ring may optionally be substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy.

In another embodiment, X is N(R⁶)₂, m is 0 or 1, and R⁴ is R⁸—(S(O)₂)— as set forth above, wherein said R⁸ is selected from the group of substituents consisting of alkyl, alkenyl, heteroaryl, and aryl;

wherein said alkyl and alkenyl substitutents may optionally be independently substituted with one to four aryl moieties;

wherein said heteroaryl substitutent may optionally be substituted with one to four moieties independently selected from the group consisting of halo, alkyl, heteroaryl, alkyl-(C═O)—NH—, and alkyl-O—(C═O)—.

wherein said aryl substitutent may optionally be substituted with one to four moieties independently selected from the group consisting of alkyl, aryl, halo, cyano, alkoxy, alkyl-(C═O)—, alkyl-O—(C═O)—, alkyl-S(O)₂—, perhaloalkyl, perhaloalkoxy, and aryloxy; wherein when each of said aryl and heteroaryl substituents contains two moieties on adjacent carbon atoms anywhere within said substituent, such moieties may optionally and independently in each occurrence, be taken together with the carbon atoms to which they are attached to form a five to six membered carbocyclic or heterocyclic ring which carbocyclic or heterocyclic ring may optionally be independently substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy;

wherein said aryl moiety of said alkyl and alkenyl substituents may optionally be substituted with one or two radicals selected from the group consisting of alkyl, cyano, halo, aryl, and perhaloalkyl.

In another embodiment, X is N(R⁶)₂, m is 0 or 1, and R⁴ is R⁸—(S(O)₂)— as set forth above, wherein said R⁸ heteroaryl substituent, including heteroaryl substituent containing two moieties on adjacent carbon atoms which are taken together with the carbon atoms to which said moieties are attached to form a five to six membered carbocyclic or heterocyclic ring, is selected from the group consisting of pyridinyl, furanyl, thiophenyl, pyrrolyl, 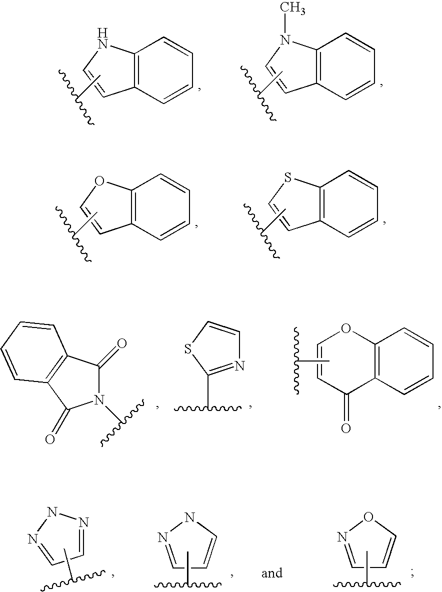
each of which may be optionally substituted.

In another embodiment, X is N(R⁶)₂, m is 0 or 1, and R⁴ is R⁸—(S(O)₂)— as set forth above, wherein said R⁸ aryl substituent, including R⁸ aryl substituent containing two moieties on adjacent carbon atoms which are taken together with the carbon atoms to which said moieties are attached to form a five to six membered carbocyclic or heterocyclic ring, is selected from the group consisting of phenyl, naphthyl, 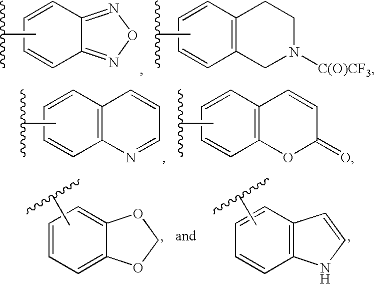
each of which may be optionally substituted.

In another embodiment, is N(R⁶)₂, m is 0 or 1, and R⁴ is —(C═O)—NR⁹R¹⁰—; wherein R⁹ is selected from the group of substituents consisting of alkyl, alkenyl, alkynyl, cycloalkyl, cycloalkenyl, aryl, heteroaryl, and heterocyclyl, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;

wherein each of said aryl, cycloalkyl, heterocyclyl, and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said radicals containing an aryl alternative may optionally be substituted by one or two groups independently selected from the group consisting of alkyl, halo, alkoxy, cyano, perhaloalkyl and perhaloalkoxy; and

R¹⁰ is selected from the group consisting of hydrogen or alkyl.

In another embodiment, X is N(R⁶)₂, m is 0 or 1, and R⁴ is —(C═O)—NR⁹R¹⁰; wherein R⁹ is selected from the group of substituents consisting of alkyl, cycloalkyl, and aryl;

wherein said alkyl substituent may optionally be substituted with one to four moieties independently selected from the group consisting of halo, alkoxy, hydroxyl, perhaloalkyl and aryl; wherein said aryl moiety may optionally be substituted with one or two radicals selected independently from the group consisting of alkyl, cyano, halo, aryl, and perhaloalkyl;

said cycloalkyl substitutent may optionally be substituted with one to four moieties independently selected from the group consisting of aryl, halo, alkyl, and alkoxy;

said aryl substitutent may optionally be substituted with one to four moieties independently selected from the group consisting of halo, alkyl, cyano, alkoxy, perhaloalkyl, nitro, and aryl; wherein when said aryl substituent contains two moieties on adjacent carbon atoms anywhere within said substituent, such moieties may optionally and independently in each occurrence, be taken together with the carbon atoms to which they are attached, to form a five to six membered carbocyclic or heterocyclic ring; and

R¹⁰ is selected from the group consisting of hydrogen or alkyl.

In another embodiment, X is N(R⁶)₂, m is 0 or 1, and R⁴ is —(C═O)—NR⁹R¹⁰ as set forth above, wherein the R⁹ cycloalkyl substituent is selected from the group consisting of cyclopropyl, cyclobutyl, cyclopenyl, cyclohexyl, and cycloheptyl, each of which may optionally be substituted.

In another embodiment, X is N(R⁶)₂, m is 0 or 1, and R⁴ is —(C═O)—NR⁹R¹⁰ as set forth above, wherein the R⁹ aryl substituent, including R⁹ aryl substituent containing two moieties on adjacent carbon atoms which are taken together with the carbon atoms to which said moieties are attached to form a five to six membered carbocyclic or heterocyclic ring, is selected from the group consisting of phenyl, naphthyl, 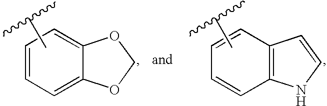
each of which may be optionally substituted.

In another embodiment, X is N(R⁶)₂, and m is 0 or 1, wherein one R⁶ is selected from the group of substituents consisting of hydrogen or alkyl, and the other R⁶ is selected from the group of substituents consisting of alkyl, cycloalkyl, heterocyclyl, heteroaryl and aryl; wherein each of the aforesaid other R⁶ alkyl, cycloalkyl, heterocyclyl, heteroaryl and aryl substituents may optionally be independently substituted by one to four moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl and aryl substituent contains two moieties on adjacent carbon atoms anywhere within said substituent, such moieties may optionally and independently in each occurence be taken together with the carbon atoms to which they are attached to form a five to six membered carbocyclic or heterocyclic ring;

wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy.

In another embodiment, X is N(R⁶)₂, m is 0 or 1, and one R⁶ is selected from the group of substituents consisting of hydrogen or alkyl, and the other R⁶ is selected from the group of substituents consisting of alkyl, cycloalkyl, heterocyclyl and aryl;

wherein the other R⁶ alkyl substituent is substituted by one to four moieties independently selected from the group consisting of amino, (alkyl)₂-amino, alkyl-O—(C═O)—, H₂N—(C═O)—, alkyl-O—NH—(C═O)—, alkyl-O—NH—(C═O)-alkyl-NH—(C═O)—, heterocyclyl, aryl, aryloxy and heteroaryl; wherein each of said heterocyclyl, aryl and aryloxy moieties may optionally be substituted by one or two radicals independently selected from the group consisting of alkyl, halo, cyano, alkoxy, perhaloalkyl and perhaloalkoxy; wherein when each of said aryl and aryloxy moieties contains two radicals on adjacent carbon atoms anywhere within said moiety, such radicals may optionally and independently in each occurence be taken together with the carbon atoms to which they are attached to form a five to six membered carbocyclic or heterocyclic ring;

wherein when the other R⁶ cycloalkyl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;

wherein the other R⁶ heterocyclyl substituent may obtionally be substituted with arylalkyl-;

wherein the other R⁶ aryl substituent may optionally be substituted by one to four moieties independently selected from the group consisting of alkyl, alkoxy, halo, cyano, and alkyl-S—.

In another embodiment, in formula I, X is N(R⁶)₂, m is 0 or 1, wherein one R⁶ is hydrogen, and the other R⁶ is alkyl substituted by one or two moieties selected indendently from the group consisting of alkyl-(C═O)—, H₂N—(C═O)—, amino, and (alkyl)₂-amino.

In another embodiment, in formula I, X is N(R⁶)₂, m is 0 or 1, wherein N(R⁶)₂ is selected from the group consisting of

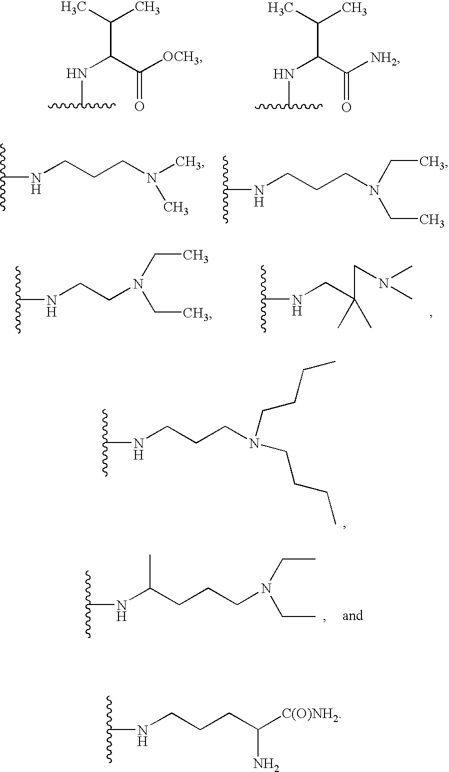

In another embodiment, in formula I, in formula I, X is N(R⁶)₂, m is 0 or 1, wherein one R⁶ is selected from the group of substituents consisting of hydrogen or alkyl, and the other R⁶ is as set forth above, wherein the heteroaryl moiety of the other R⁶ alkyl substituent is selected from the group consisting of imidazolyl, pyridinyl, furanyl, thiophenyl, and pyrrolyl, each of which may be optionally substituted.

In another embodiment, in formula I, in formula I, X is N(R⁶)₂, m is 0 or 1, wherein one R⁶ is selected from the group of substituents consisting of hydrogen or alkyl, and the other R⁶ is as set forth above, wherein the heterocyclyl moiety of the other R⁶ alkyl substituent is selected from the group consisting of tetrahydrofuranyl, tetrahydropyranyl, tetrahydrothiophenyl, tetrahydrothiopyranyl, piperidinyl, morpholinyl, piperizinyl, and pyrrolidinyl, each of which may be optionally substituted.

In another embodiment, in formula I, in formula I, X is N(R⁶)₂, m is 0 or 1, wherein one R⁶ is selected from the group of substituents consisting of hydrogen or alkyl, and the other R⁶ is as set forth above, wherein the aryl and aryloxy moieties of the other R⁶ alkyl substituent including aryl and aryloxy moieties containing two radicals on adjacent carbon atoms which are taken together with the carbon atoms to which said radicals are attached to form a five to six membered carbocyclic or heterocyclic ring, is selected from the group consisting of phenyl, phenyloxy, naphthyl, naphthyloxy, 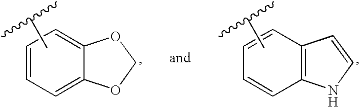
each of which may optionally be substituted.

In another embodiment, in formula I, in formula I, X is N(R⁶)₂, m is 0 or 1, wherein the two R⁶ groups of N(R⁶)₂ are taken together with the nitrogen atom to which they are shown attached to form a heterocyclyl or heteroaryl ring which heterocyclyl or heteroaryl ring may optionally be independently substituted with one to two substituents independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;

wherein each of the aforesaid alkyl, alkenyl, aryl, arylalkyl-, cycloalkyl, heteroaryl, and heterocyclyl substituents may optionally be independently substituted with one to two moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl, heterocyclycl, heteroaryl and aryl moieties contains two radicals on adjacent carbon atoms anywhere within said moiety, such radicals may optionally and independently in each occurrence, be taken together with the carbon atoms to which they are attached to form a five to six membered carbocyclic or heterocyclic ring; wherein when each of said cycloalkyl, heterocyclycl, heteroaryl and aryl moieties contains two radicals on the same carbon, such radicals may optionally be taken together with the carbon atom to which they are attached to form a five to six membered carbocyclic or heterocyclic ring; wherein each of the aforesaid moieties containing an aryl alternative may optionally be substituted by one or two radicals independently selected from the group consisting of alkyl, halo, alkoxy, cyano, perhaloalkyl and perhaloalkoxy.

In another embodiment, in formula I, X is N(R⁶)₂, m is 0 or 1, wherein the two R⁶ groups of N(R⁶)₂ are taken together with the nitrogen atom to which they are shown attached form a heterocyclyl or heteroaryl ring which heterocyclyl or heteroaryl ring may optionally be independently subsitiuted with one to two substituents independently selected from the group consisting of halo, alkyl, hydroxy, cycloalkyl, aryl, heteroaryl, heterocyclyl, aryl-(C═O)—, heterocyclyl-(C═O)—, heteroaryl-(C═O)—, arylalkyl-O—(C═O)—, (alkyl)₂-N—(C═O)—, (alkyl)₂-amino, H₂N—(C═O)—, alkyl-O—(C═O)—, and alkyl-(C═O)—; wherein when said heterocyclyl or heteroaryl ring formed by said N(R⁶)₂ contains two moieties on adjacent carbon atoms anywhere within said heterocyclyl or heteroaryl ring, such moieties may optionally and independently in each occurrence, be taken together with the carbon atoms to which they are attached to form a five to six membered carbocyclic or heterocyclic ring;

wherein when each of said cycloalkyl, aryl, heteroaryl, and heterocyclyl substituents of said heterocyclyl or heteroaryl ring contains two moieties on adjacent carbon atoms anywhere within said substituent, such moieties may optionally and independently in each occurrence, be taken together with the carbon atoms to which they are attached, to form a five to six membered carbocyclic or heterocyclic ring;

wherein the alkyl substituent may optionally be substituted with one to two moieties independently selected from the group consisting of alkoxy, halo, and aryl moieties wherein each of said aryl moieties may optionally be substituted with one to two radicals independently selected from the group consisting of alkyl, cyano, halo, perhaloalkyl, and perhaloalkoxy; wherein when said aryl moiety contains two radicals on adjacent carbon atoms anywhere within said aryl moiety, such radicals may optionally and independently in each occurrence, be taken together with the carbon atoms to which they are attached to form a five to six membered carbocyclic or heterocyclyl ring;

wherein the aryl substituent may optionally be substituted with one or two moieties independently selected from the group consisting of halo, and perhaloalkyl;

wherein when the heterocyclyl substituent contains two moieties on the same carbon atom, such moieties may optionally be taken together with the carbon atom to which they are attached to form a five or six membered carbocyclic or heterocyclic ring.

In another embodiment, in formula I, X is N(R⁶)₂, m is 0 or 1, wherein the two R⁶ groups of N(R⁶)₂ are taken together with the nitrogen atom to which they are shown attached form a heterocyclyl ring, wherein said heterocyclyl ring, including heterocyclyl ring containing two moieties on adjacent carbon atoms which are taken together with the carbon atoms to which they are attached to form a five to six membered carbocyclic or heterocyclyl ring, is selected from the group consisting of pyrrolidinyl, morpholinyl, hexamethyleneiminyl, piperizinyl, piperidinyl, thiomorpholinyl, azacyclopropyl, homopiperizinyl, thiazolidinyl, 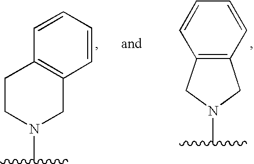
each of which may be optionally substituted.

In another embodiment, in formula I, X is N(R⁶)₂, m is 0 or 1, wherein the two R⁶ groups of N(R⁶)₂ are taken together with the nitrogen atom to which they are shown attached form a heteroaryl ring, wherein said heteraryl ring, including heteroaryl ring containing two moieties on adjacent carbon atoms which are taken together with the carbon atoms to which they are attached to form a five to six membered carbocyclic or heterocyclyl ring, is

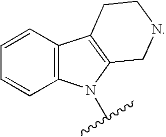

In another embodiment, the compound of formula I is selected from the group consisting of

| COMPOUND | | COMPOUND | |
|---|---|---|---|
| # | STRUCTURE | # | STRUCTURE |
| 1 | 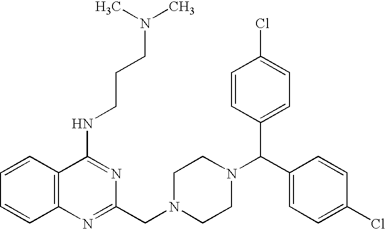 | 3 | 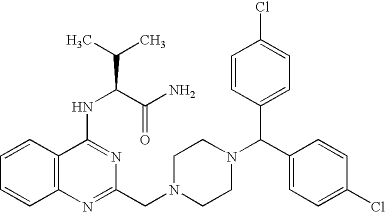 |
| 2 | 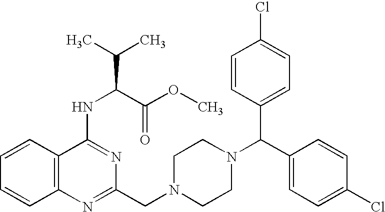 | 4-1 | 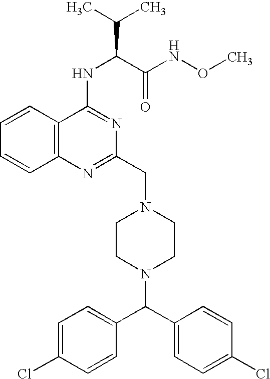 |
| 4-2 | 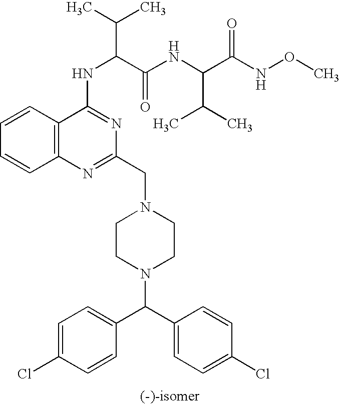 | 5-3 | 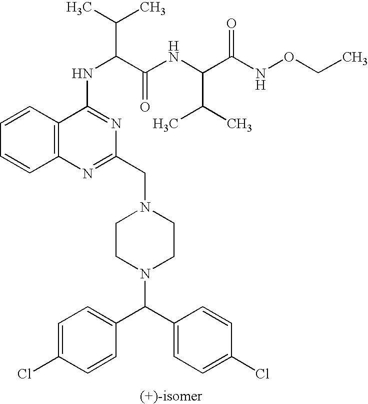 |
| 4-3 | 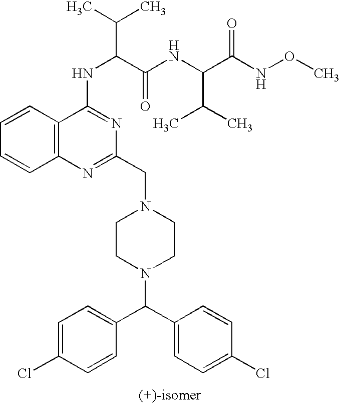 | 6 | 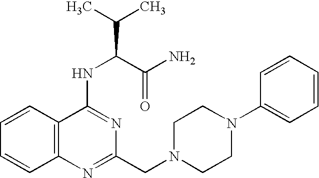 |
| 5-1 | 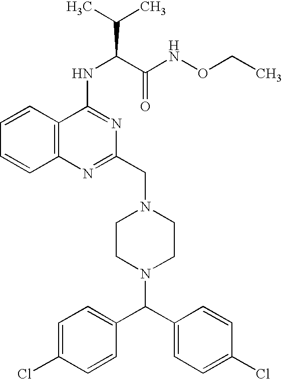 | 7 | 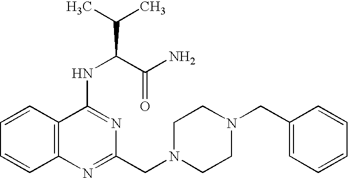 |
| 5-2 | 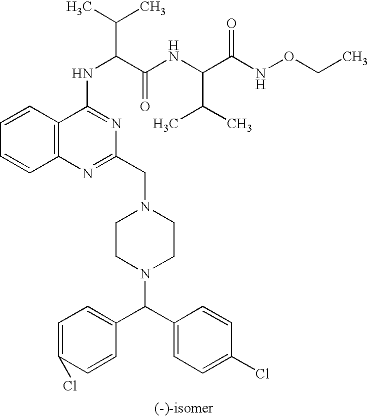 | 8 | 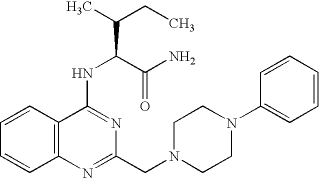 |
| 9 | 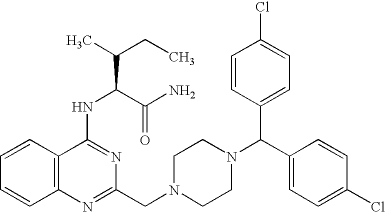 | 12-2 | 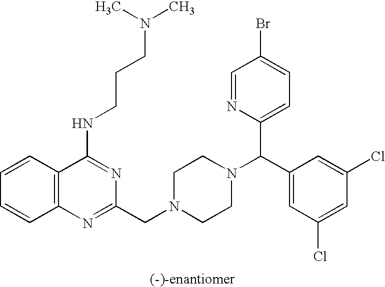 |
| 10 | 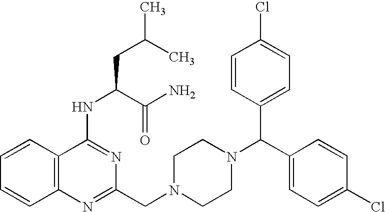 | 13-1 | 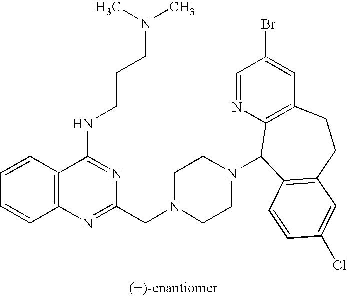 |
| 11-1 | 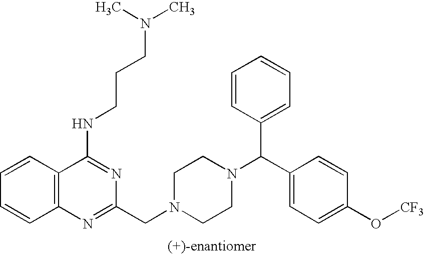 | 13-2 | 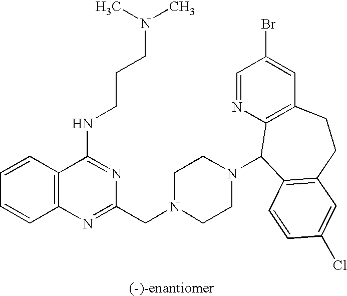 |
| 11-2 | 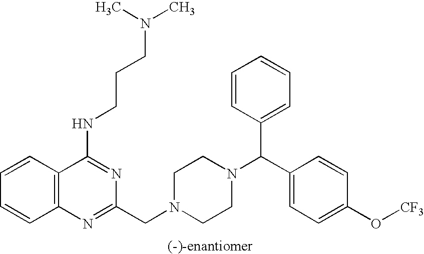 | 14 | 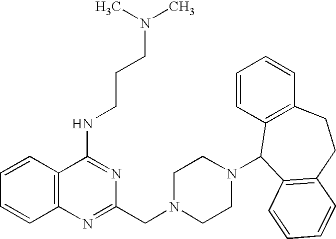 |
| 12-1 | 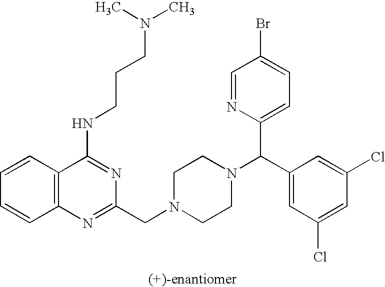 | 15 | 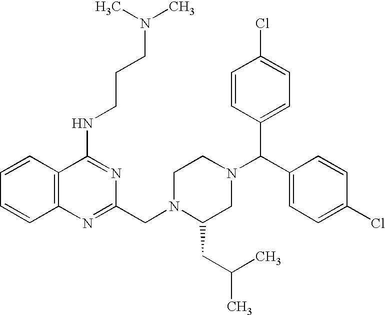 |
| 17-1 | 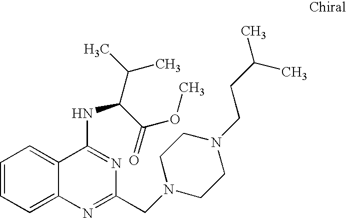 | 17-8 | 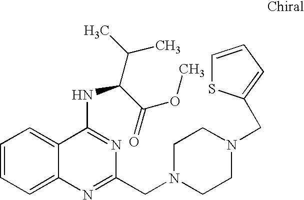 |
| 17-2 | 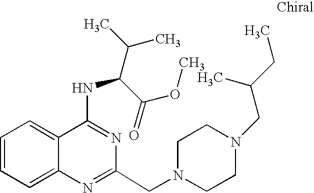 | 17-9 | 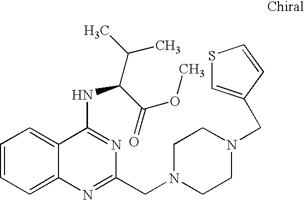 |
| 17-3 | 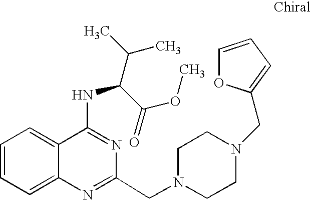 | 17-10 | 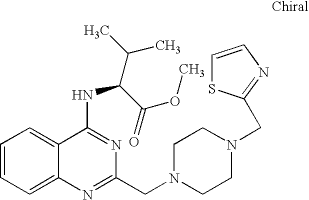 |
| 17-4 | 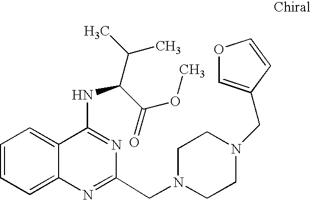 | 17-11 | 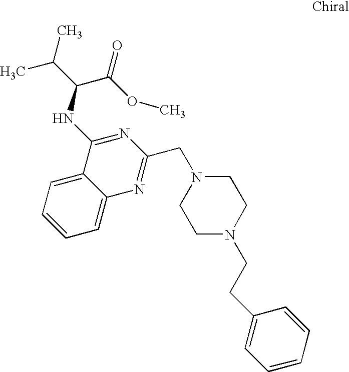 |
| 17-5 | 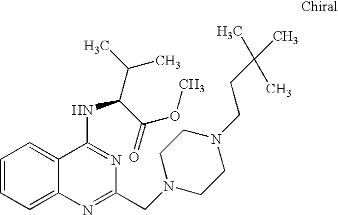 | 17-12 | 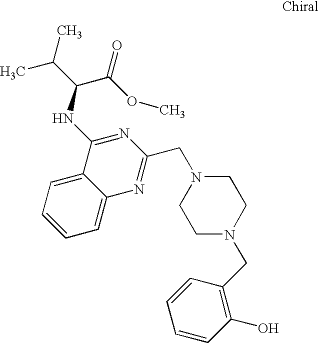 |
| 17-6 | 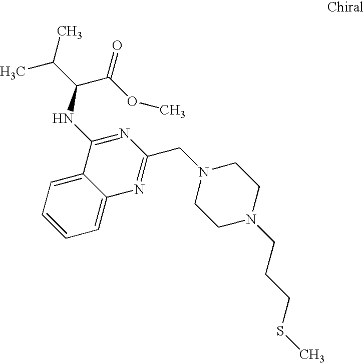 | 17-13 | 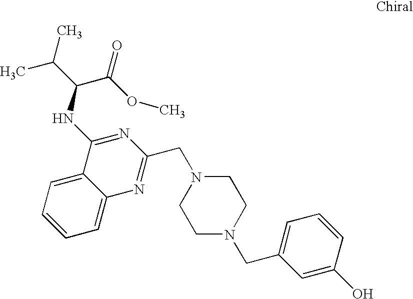 |
| 17-7 | 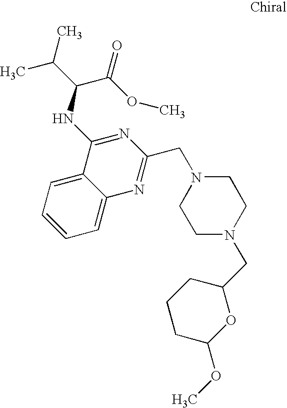 | 17-14 | 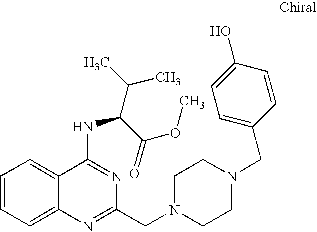 |
| 17-15 | 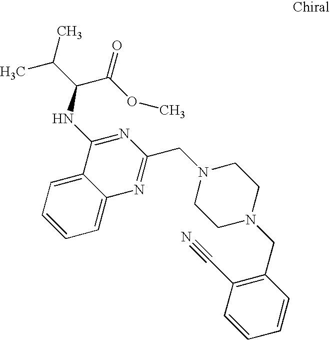 | 17-22 | 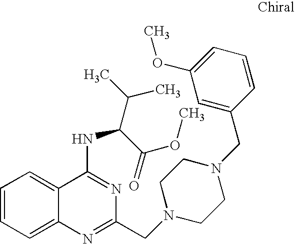 |
| 17-16 | 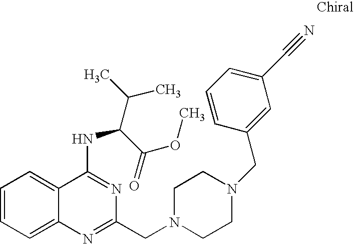 | 17-23 | 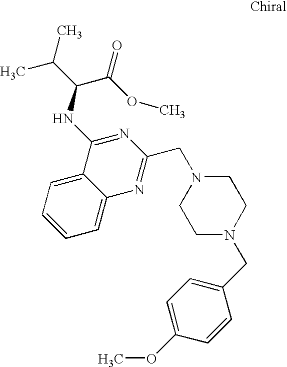 |
| 17-17 | 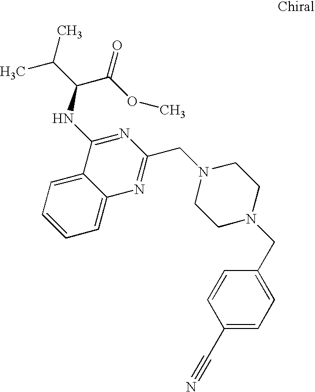 | 17-24 | 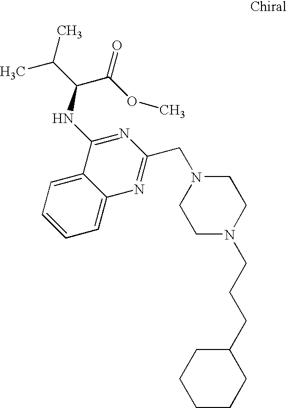 |
| 17-18 | 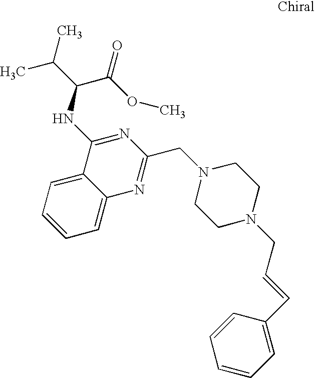 | 17-25 | 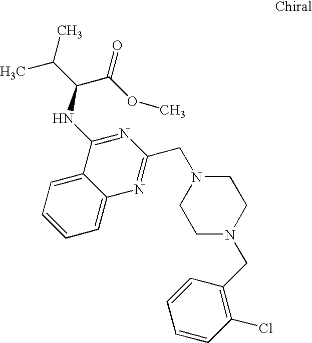 |
| 17-19 | 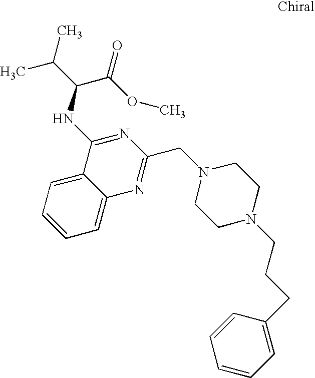 | 17-26 | 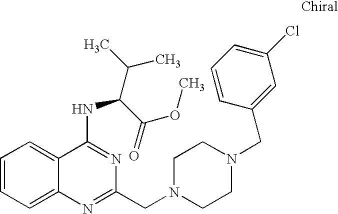 |
| 17-20 | 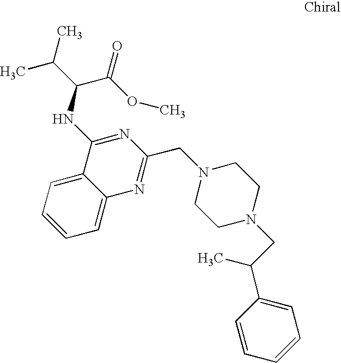 | 17-27 | 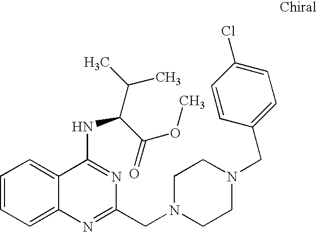 |
| 17-21 | 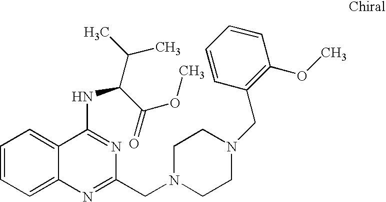 | 17-28 | 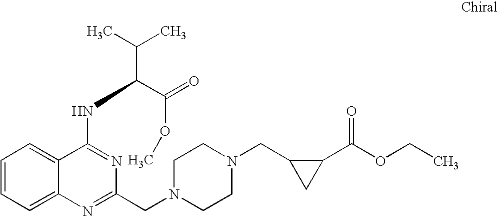 |
| 17-29 | 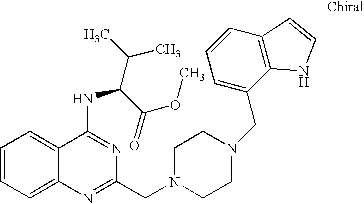 | 17-36 | 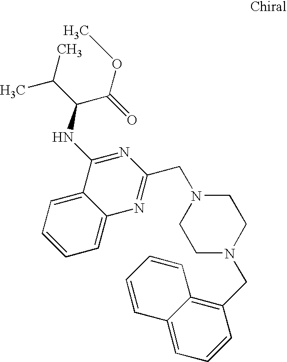 |
| 17-30 | 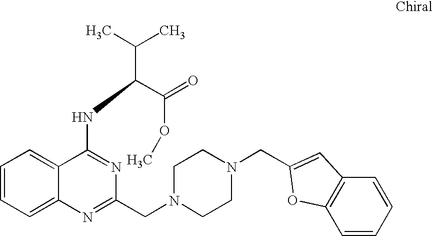 | 17-37 | 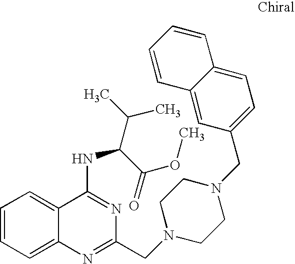 |
| 17-31 | 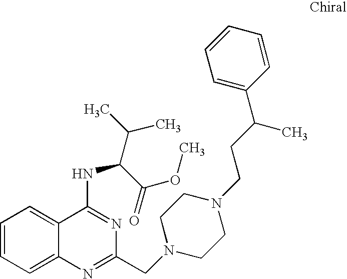 | 17-38 | 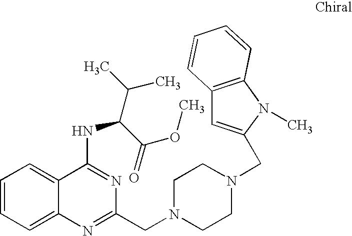 |
| 17-32 | 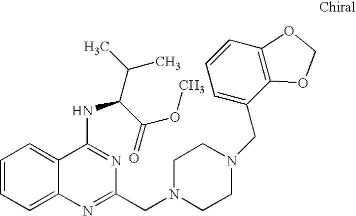 | 17-39 | 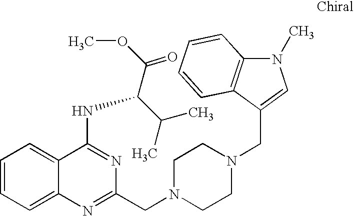 |
| 17-33 | 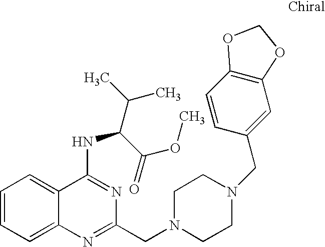 | 17-40 | 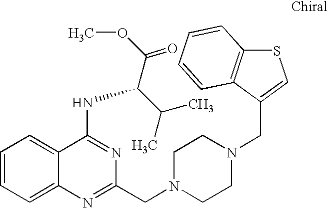 |
| 17-34 | 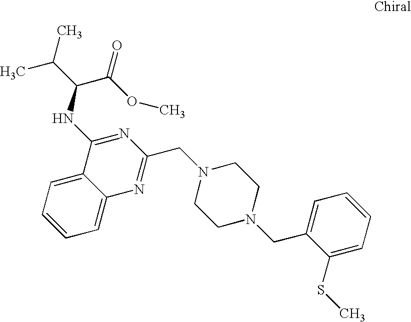 | 17-41 | 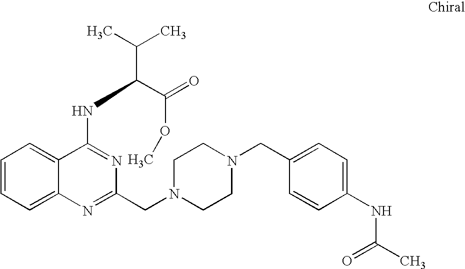 |
| 17-35 | 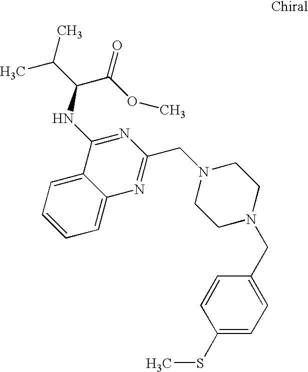 | 17-42 | 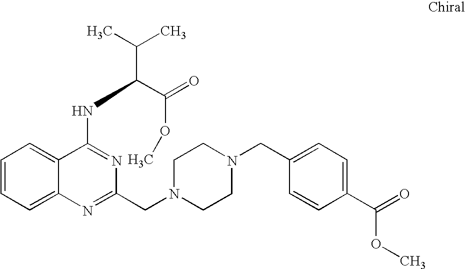 |
| 17-43 | 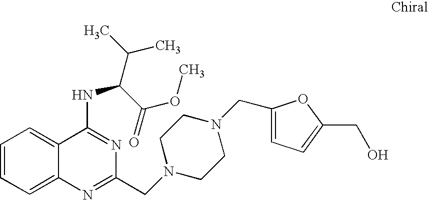 | 17-50 | 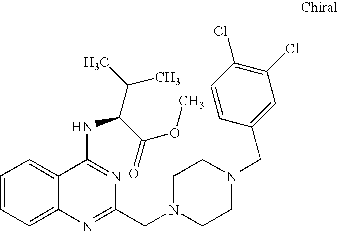 |
| 17-44 | 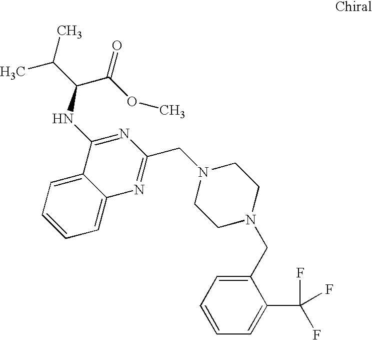 | 17-51 | 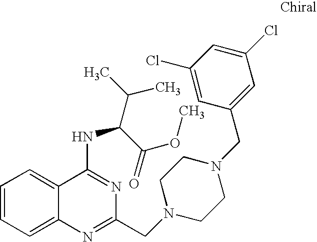 |
| 17-45 | 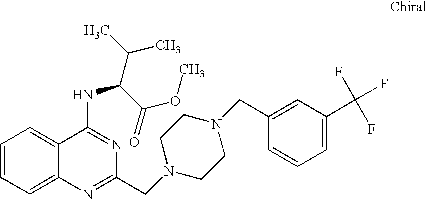 | 17-52 | 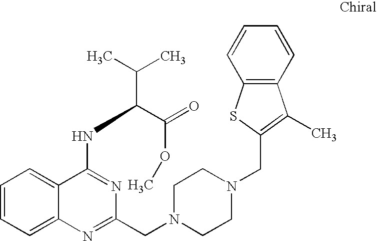 |
| 17-46 | 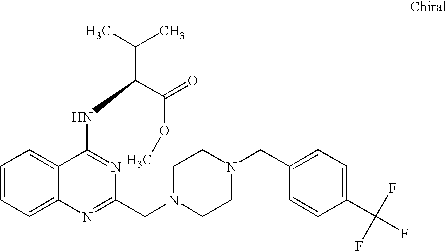 | 17-53 | 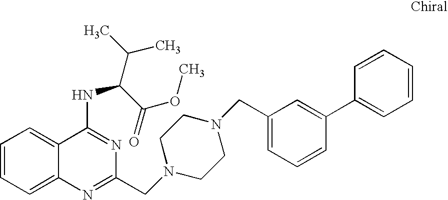 |
| 17-47 | 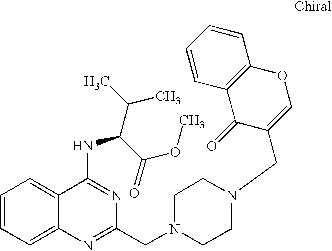 | 17-54 | 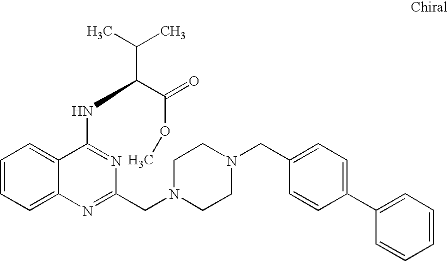 |
| 17-48 | 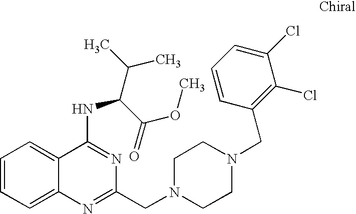 | 17-55 | 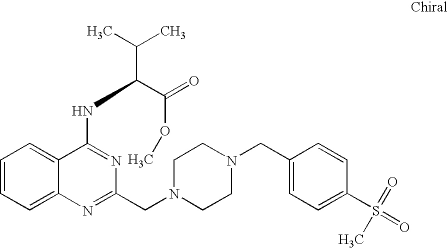 |
| 17-49 | 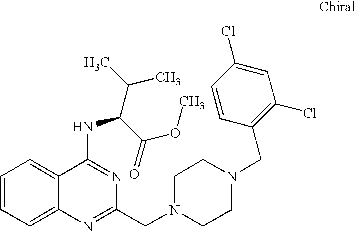 | 17-56 | 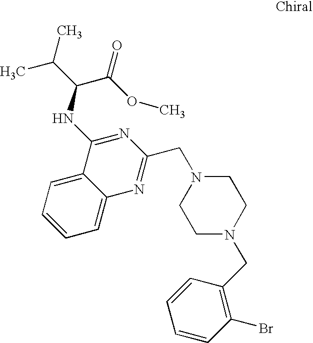 |
| 17-57 | 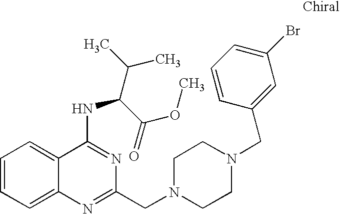 | 17-64 | 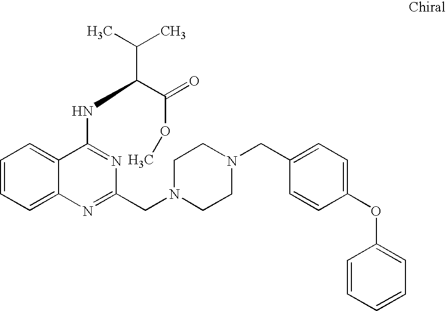 |
| 17-58 | 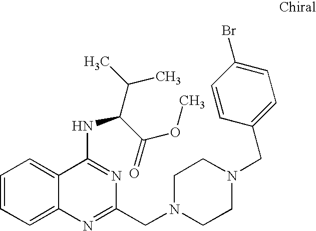 | 17-65 | 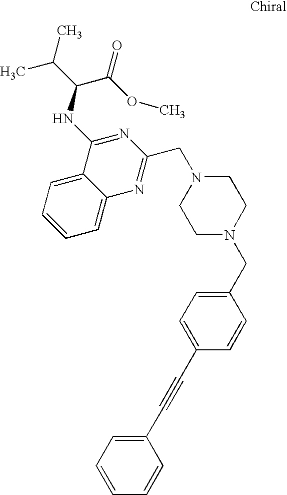 |
| 17-59 | 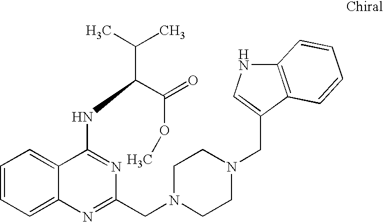 | 17-66 | 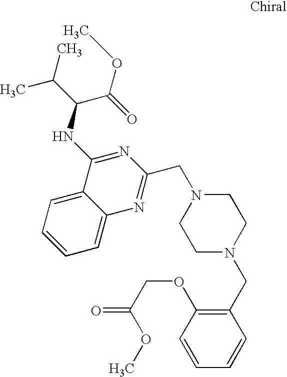 |
| 17-60 | 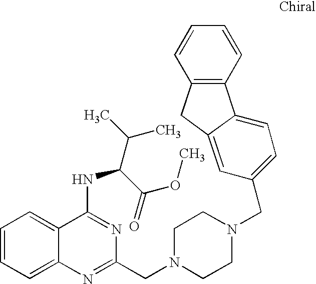 | 17-67 | 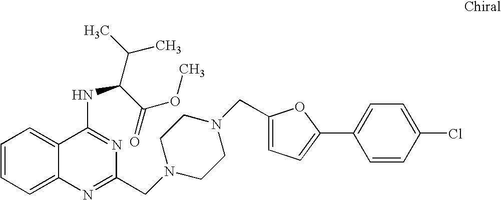 |
| 17-61 | 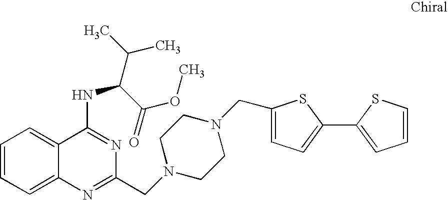 | 17-68 | 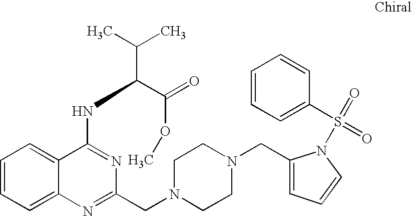 |
| 17-62 | 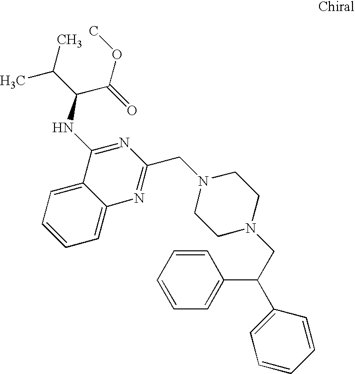 | 17-69 | 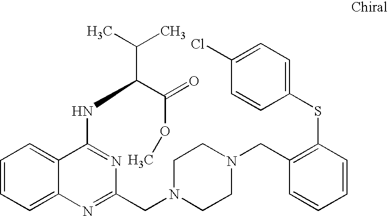 |
| 17-63 | 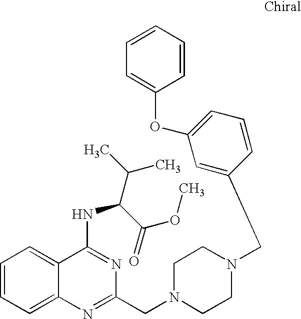 | 17-70 | 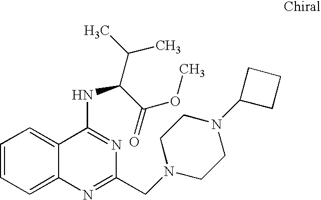 |
| 17-71 | 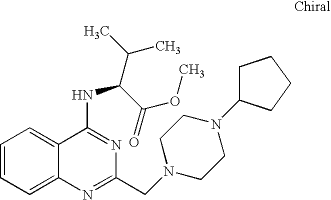 | 17-78 | 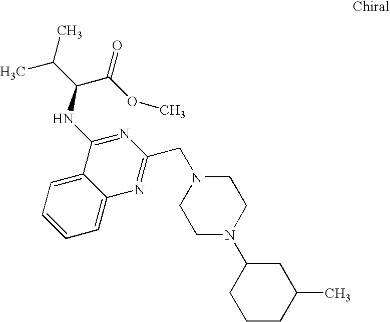 |
| 17-72 | 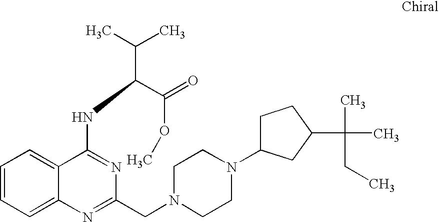 | 17-79 | 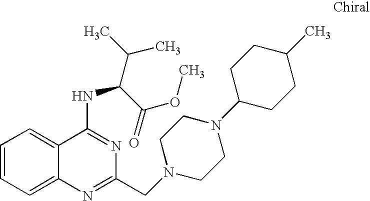 |
| 17-73 | 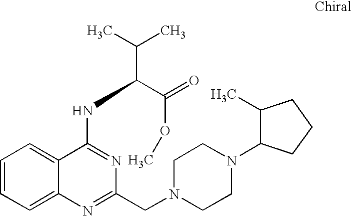 | 17-80 | 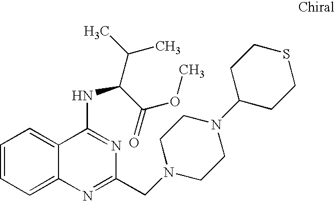 |
| 17-74 | 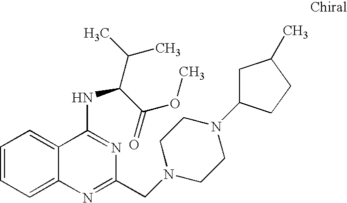 | 17-81 | 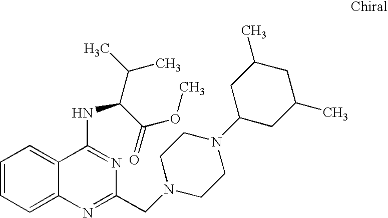 |
| 17-75 | 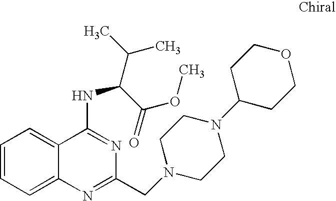 | 17-82 | 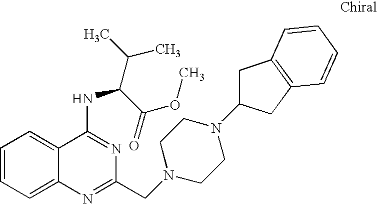 |
| 17-76 | 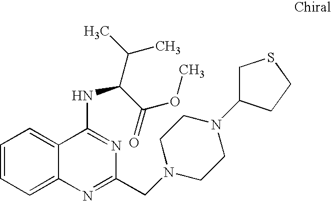 | 17-83 | 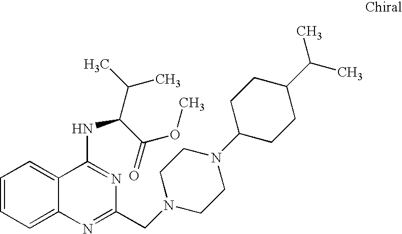 |
| 17-77 | 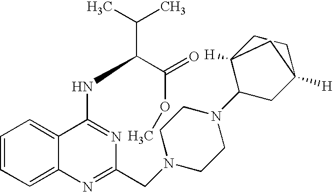 | 17-84 | 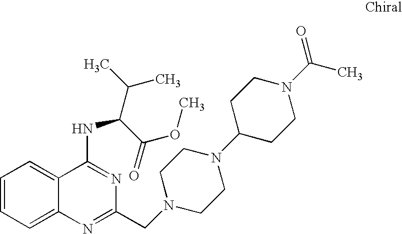 |
| 17-85 | 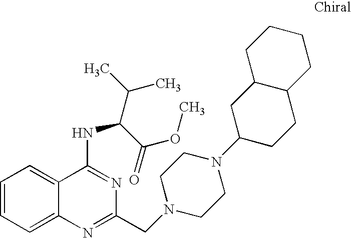 | 17-91 | 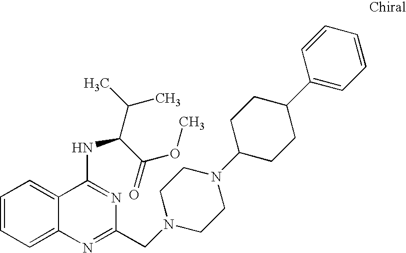 |
| 17-86 | 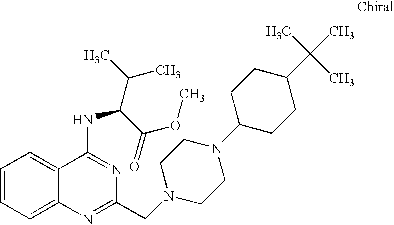 | 17-92 | 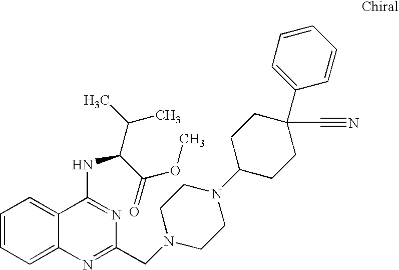 |
| 17-87 | 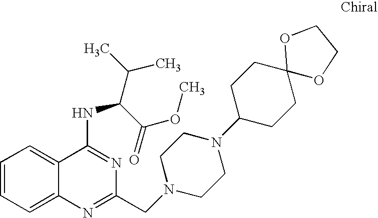 | 17-93 | 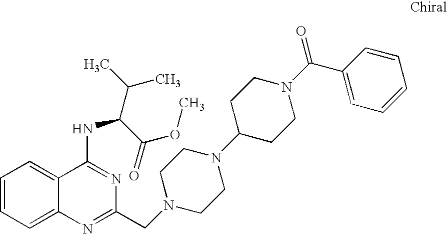 |
| 17-88 | 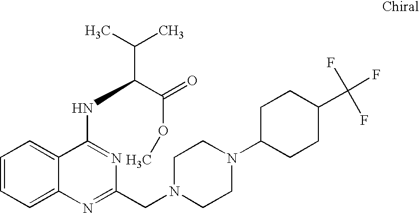 | 17-94 | 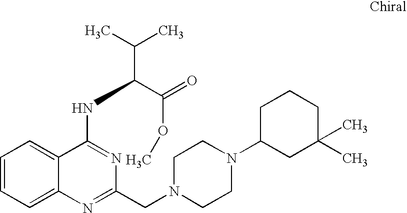 |
| 17-89 | 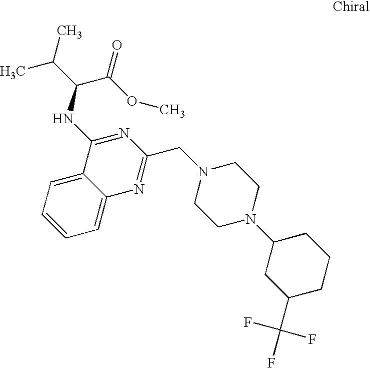 | 17-95 | 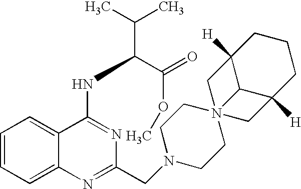 |
| 17-90 | 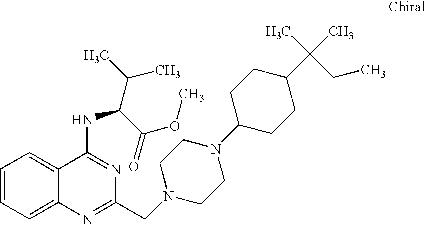 | 18-1 | 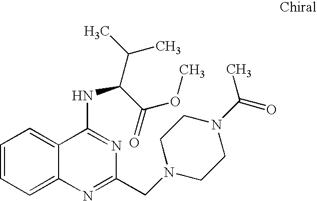 |
| 18-2 | 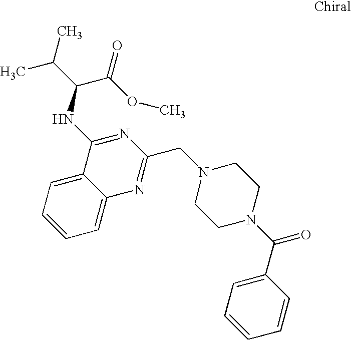 | 18-9 | 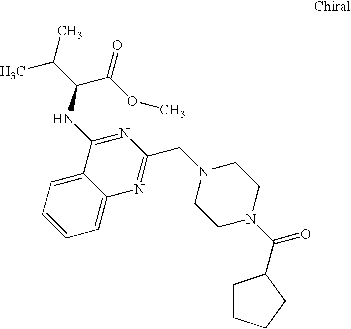 |
| 18-3 | 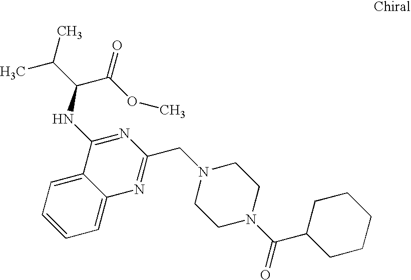 | 18-10 | 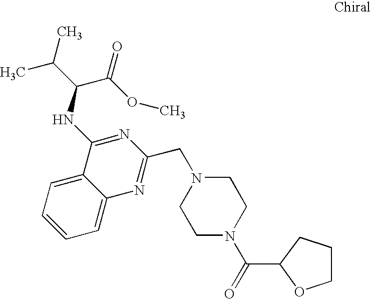 |
| 18-4 | 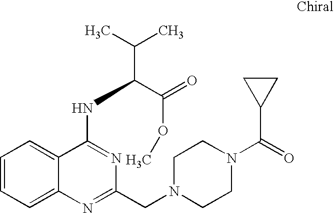 | 8-11 | 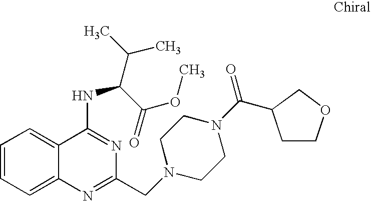 |
| 18-5 | 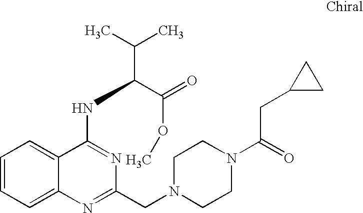 | 8-12 | 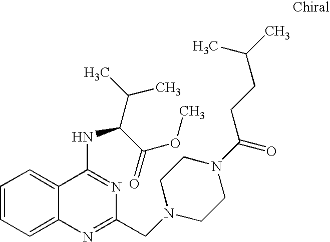 |
| 18-6 | 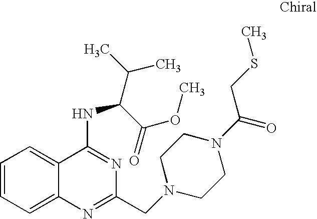 | 8-13 | 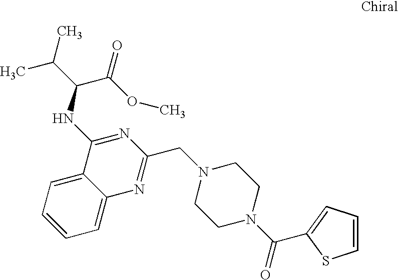 |
| 18-7 | 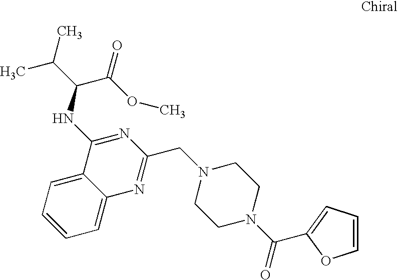 | 8-14 | 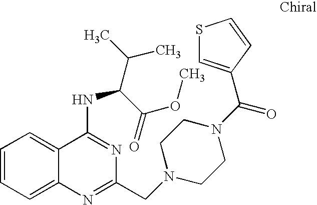 |
| 18-8 | 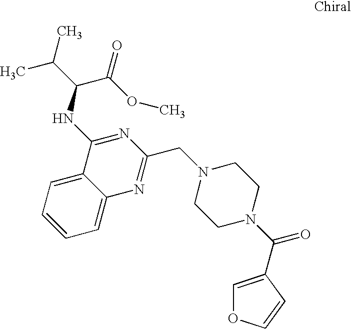 | 8-15 | 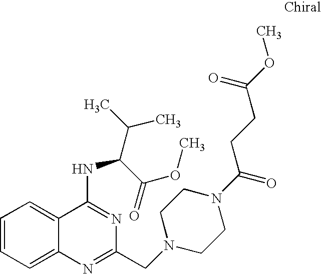 |
| 18-16 | 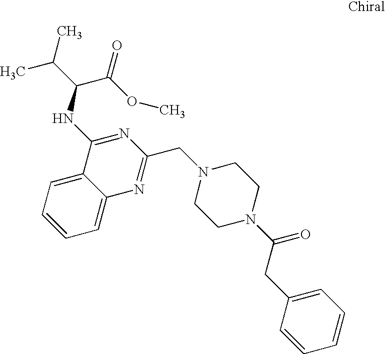 | 8-23 | 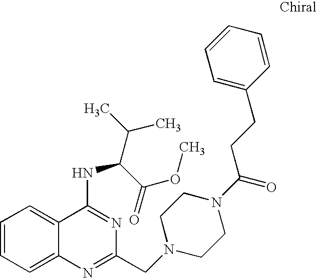 |
| 18-17 | 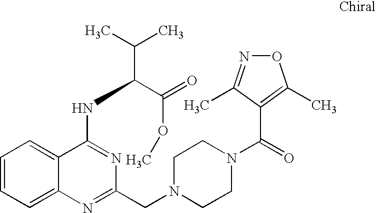 | 8-24 | 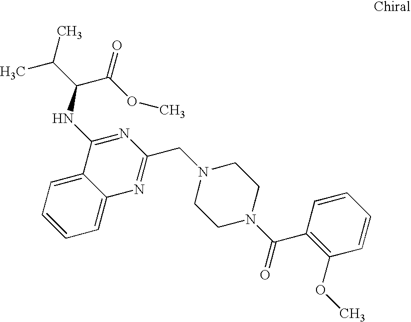 |
| 18-18 | 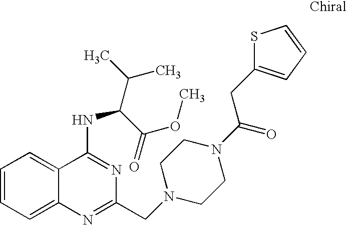 | 18-25 | 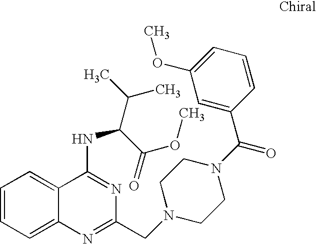 |
| 18-19 | 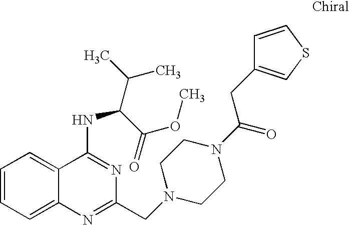 | 18-26 | 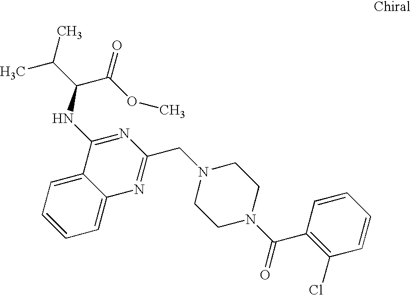 |
| 18-20 | 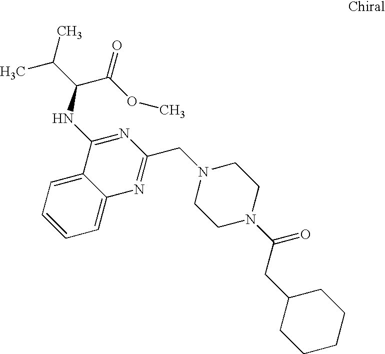 | 18-27 | 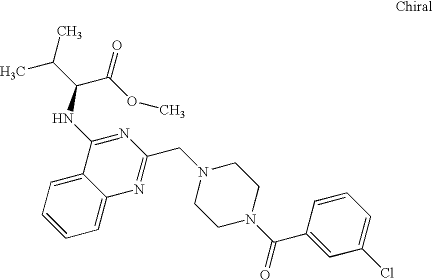 |
| 18-21 | 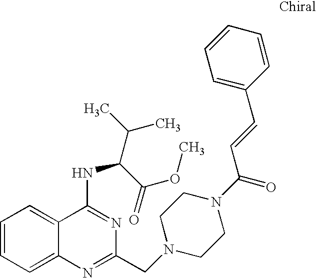 | 18-28 | 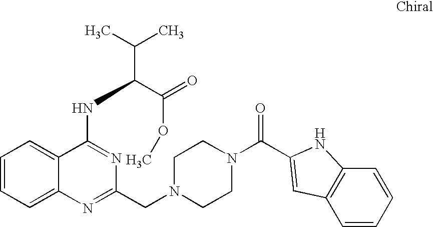 |
| 18-22 | 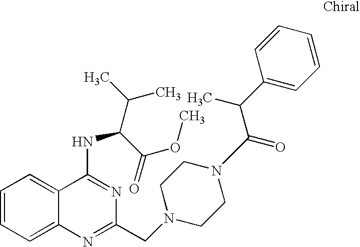 | 18-29 | 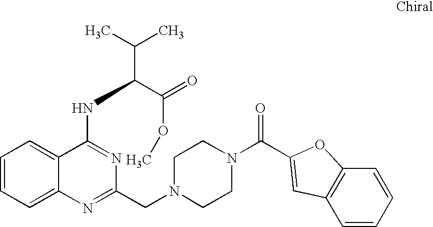 |
| 18-30 | 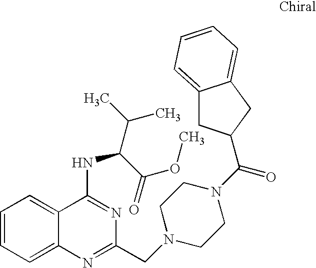 | 18-37 | 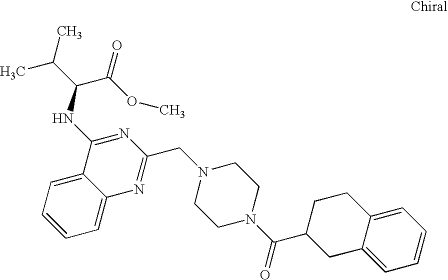 |
| 18-31 | 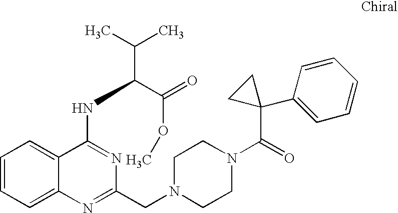 | 18-38 | 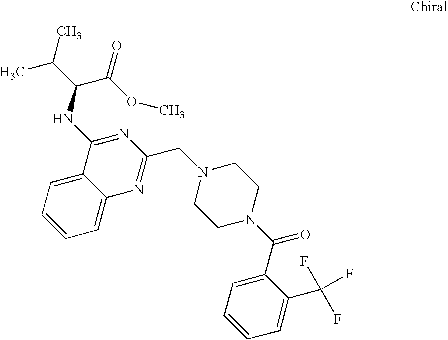 |
| 18-32 | 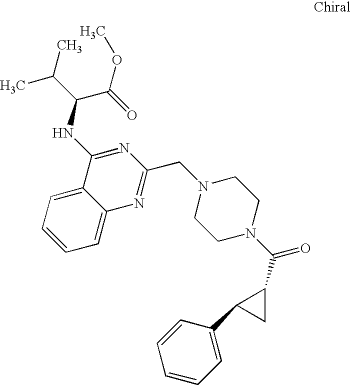 | 18-39 | 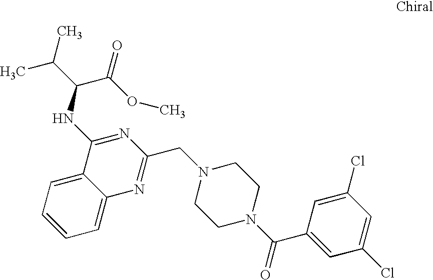 |
| 18-33 | 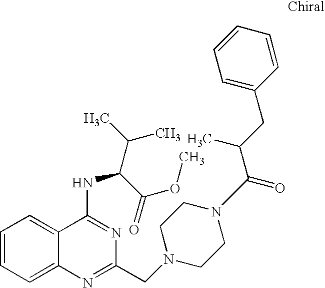 | 18-40 | 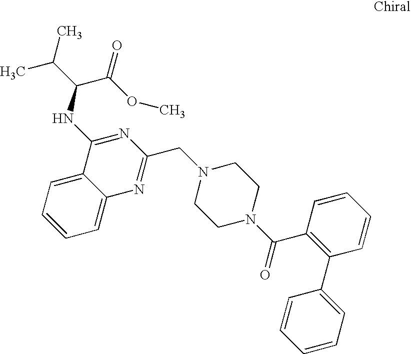 |
| 18-34 | 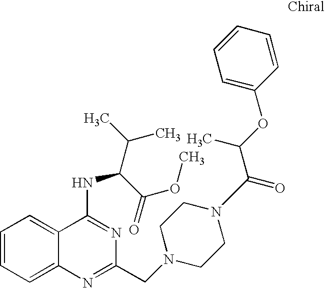 | 18-41 | 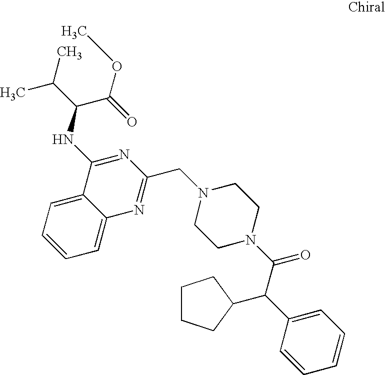 |
| 18-35 | 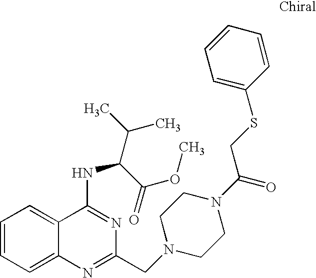 | 18-42 | 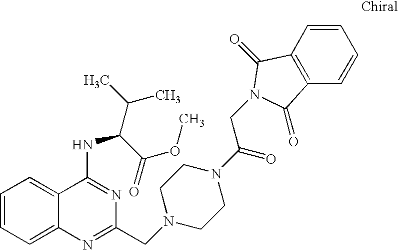 |
| 18-36 | 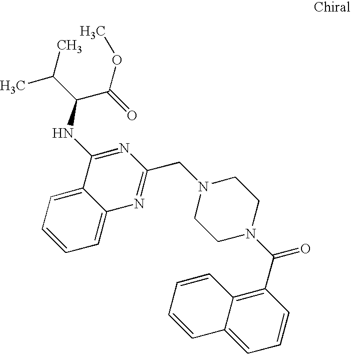 | 18-43 | 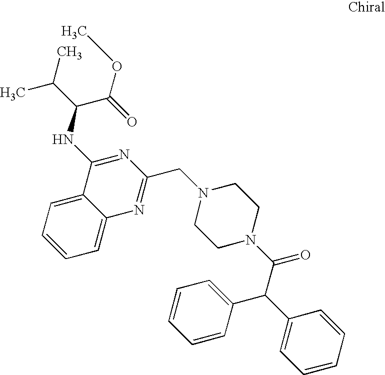 |
| 18-44 | 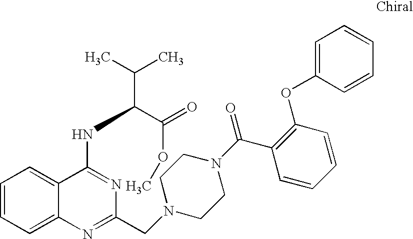 | 18-51 | 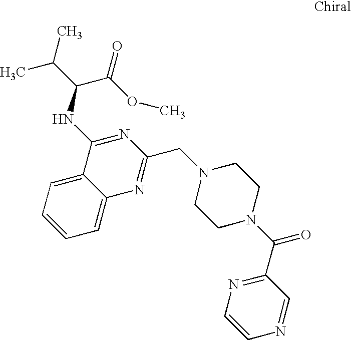 |
| 18-45 | 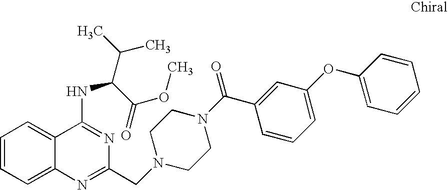 | 18-52 | 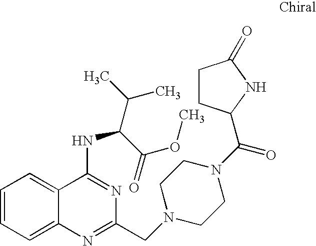 |
| 18-46 | 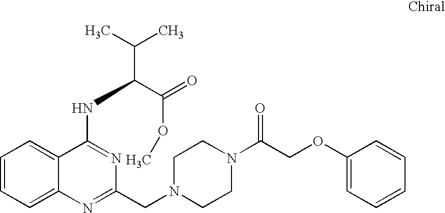 | 18-53 | 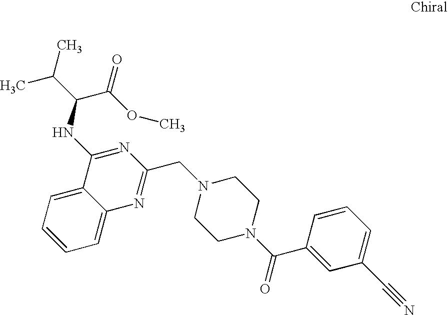 |
| 18-47 | 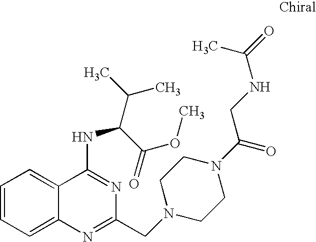 | 18-54 | 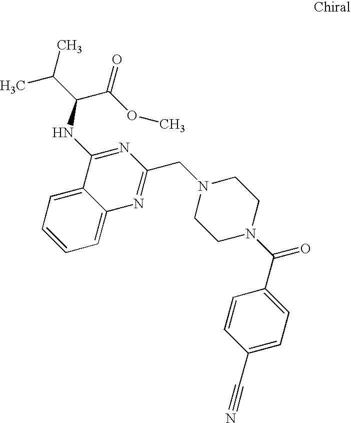 |
| 18-48 | 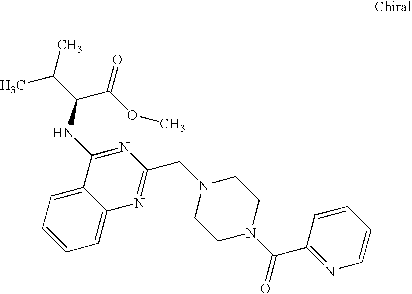 | 18-55 | 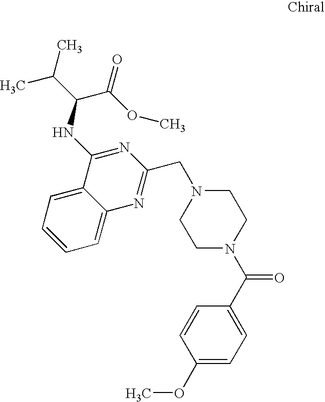 |
| 18-49 | 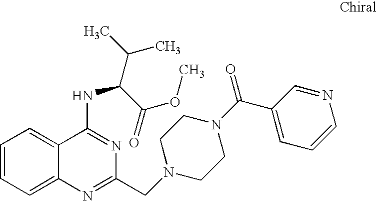 | 18-56 | 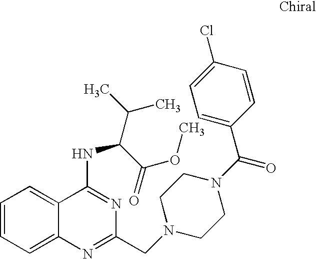 |
| 18-50 | 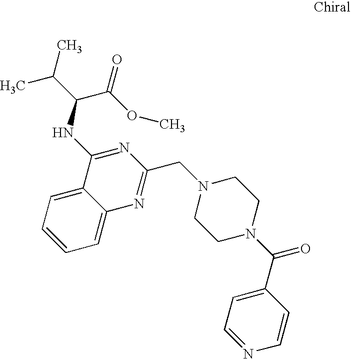 | 18-57 | 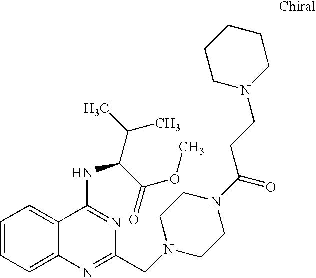 |
| 18-58 | 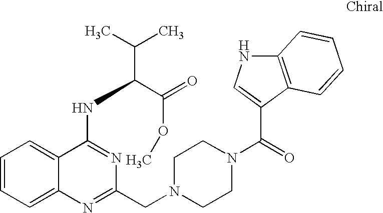 | 18-65 | 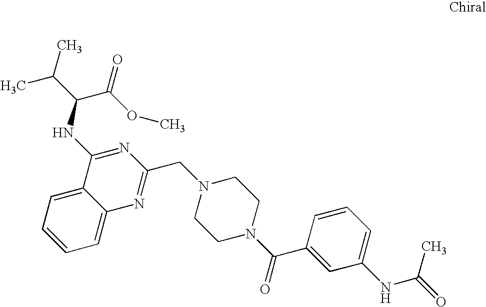 |
| 18-59 | 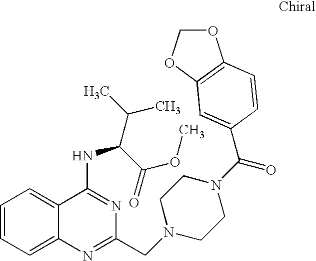 | 18-66 | 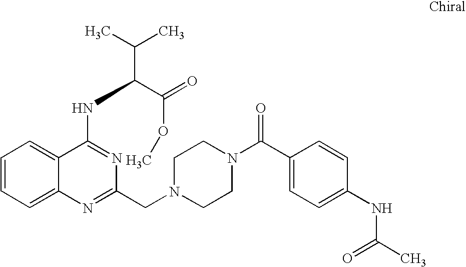 |
| 18-60 | 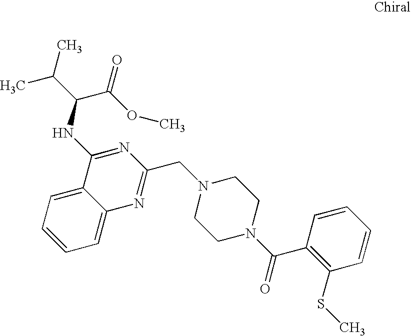 | 18-67 | 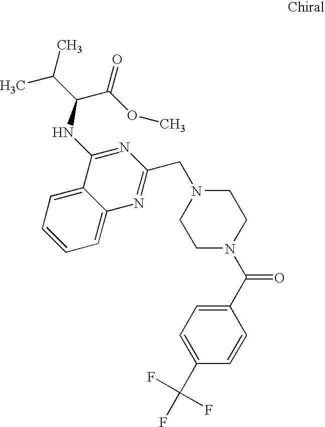 |
| 18-61 | 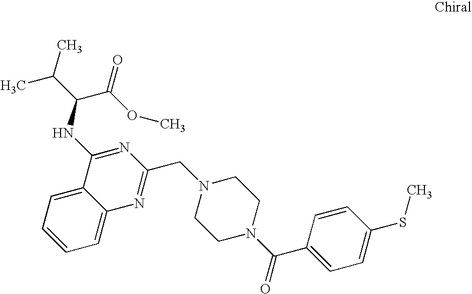 | 18-68 | 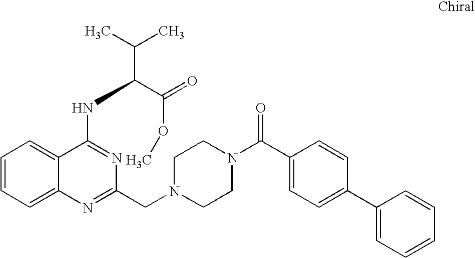 |
| 18-62 | 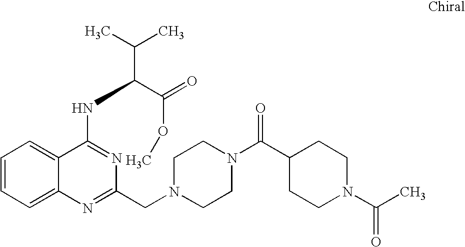 | 18-69 | 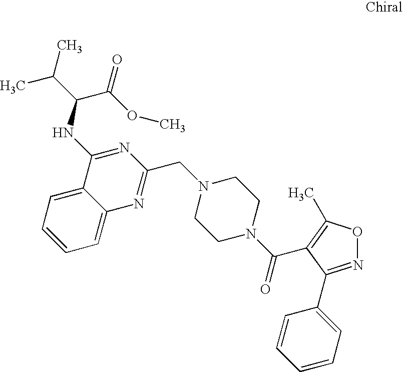 |
| 18-63 | 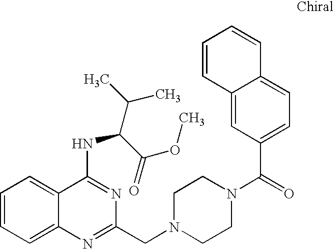 | 18-70 | 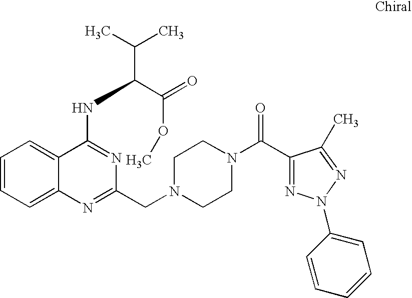 |
| 18-64 | 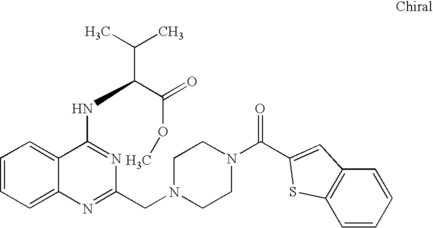 | 18-71 | 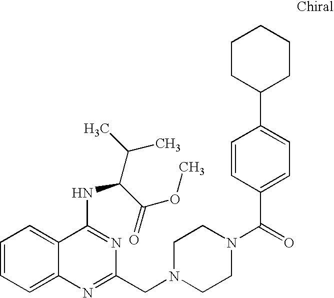 |
| 18-72 | 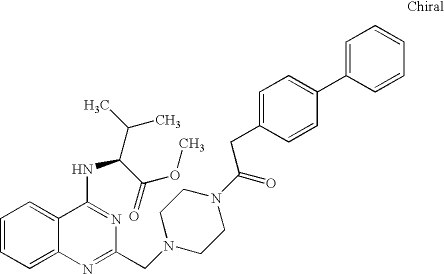 | 19-7 | 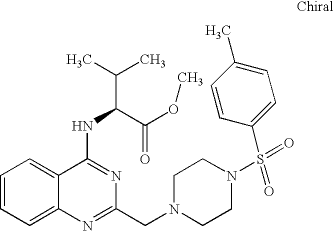 |
| 19-1 | 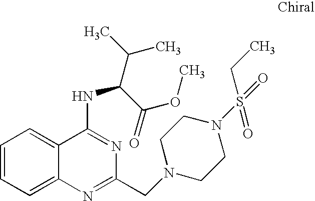 | 19-8 | 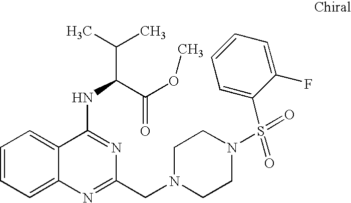 |
| 19-2 | 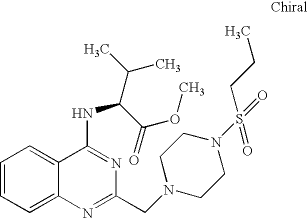 | 19-9 | 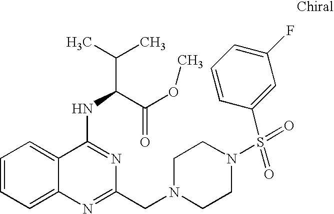 |
| 19-3 | 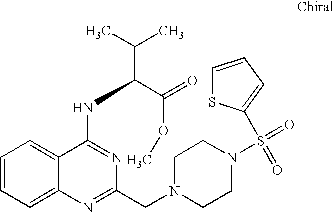 | 19-10 | 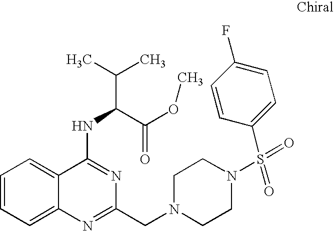 |
| 19-4 | 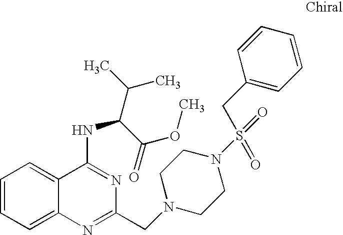 | 19-11 | 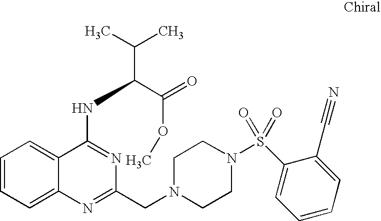 |
| 19-5 | 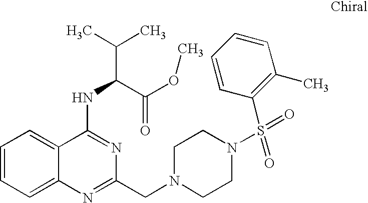 | 19-12 | 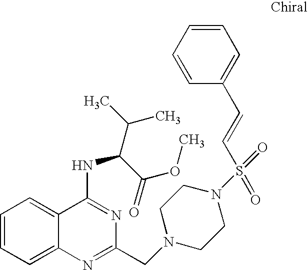 |
| 19-6 | 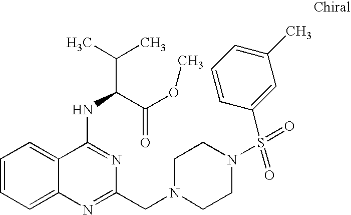 | 19-13 | 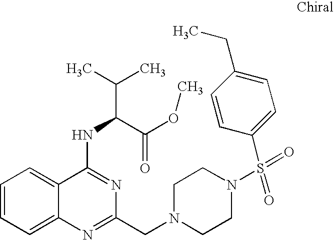 |
| 19-14 | 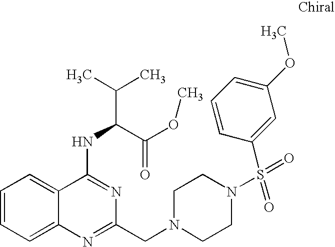 | 19-21 | 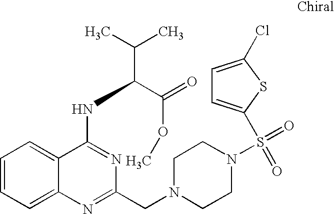 |
| 19-15 | 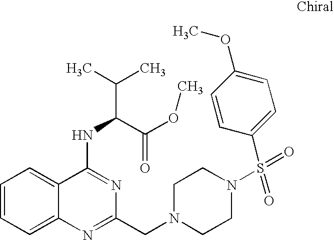 | 19-22 | 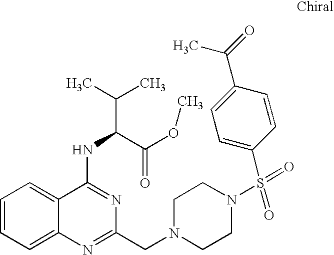 |
| 19-16 | 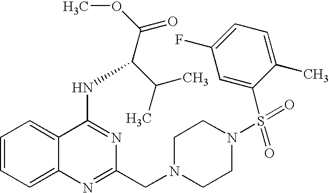 | 19-23 | 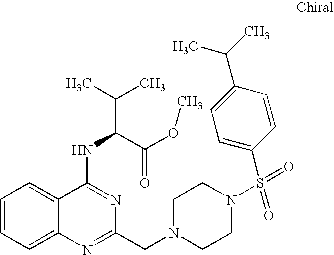 |
| 19-17 | 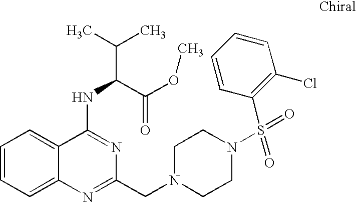 | 19-24 | 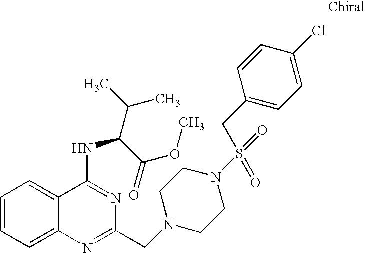 |
| 19-18 | 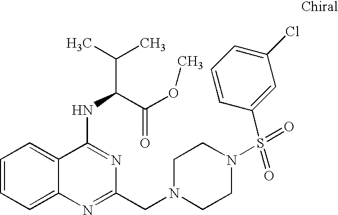 | 19-25 | 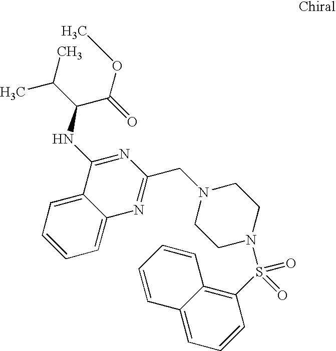 |
| 19-19 | 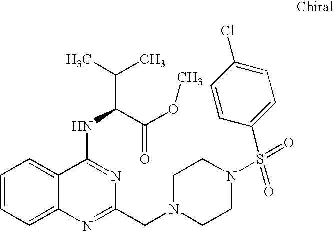 | 19-26 | 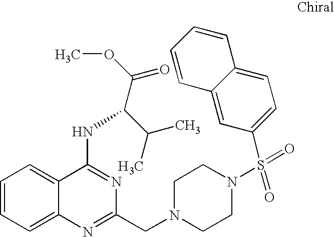 |
| 19-20 | 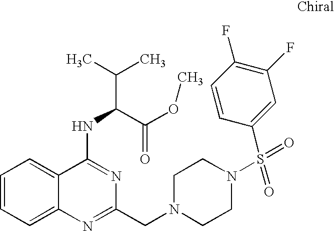 | 19-27 | 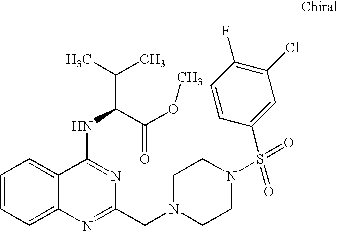 |
| 19-28 | 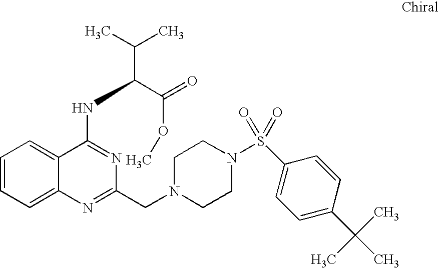 | 19-35 | 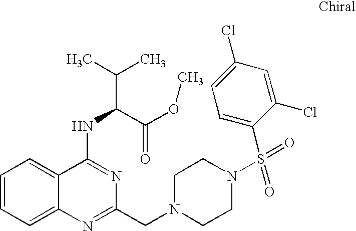 |
| 19-29 | 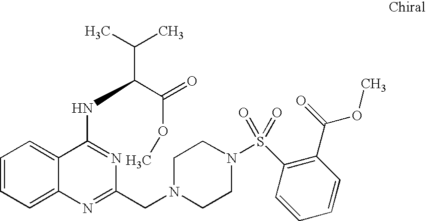 | 19-36 | 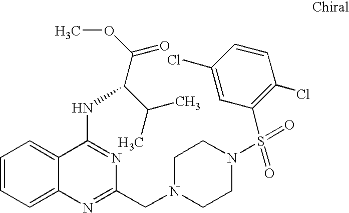 |
| 19-30 | 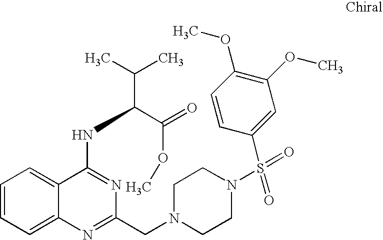 | 19-37 | 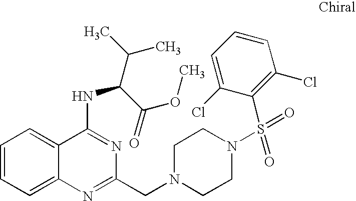 |
| 19-31 | 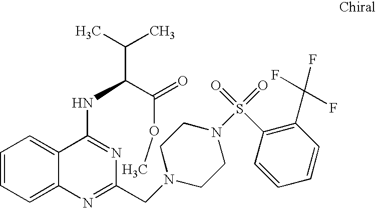 | 19-38 | 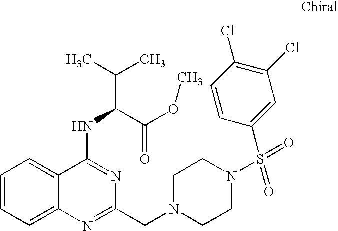 |
| 19-32 | 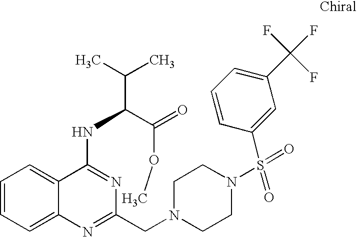 | 19-39 | 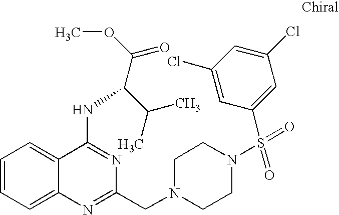 |
| 19-33 | 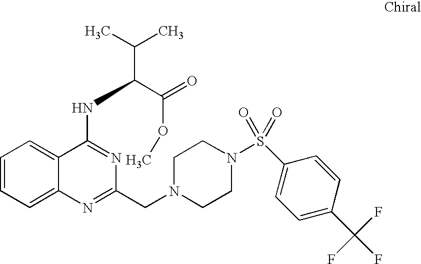 | 19-40 | 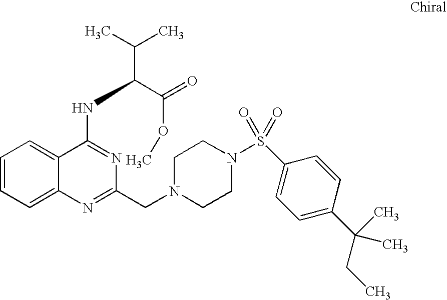 |
| 19-34 | 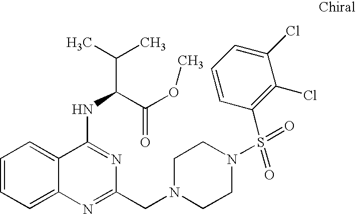 | 19-41 | 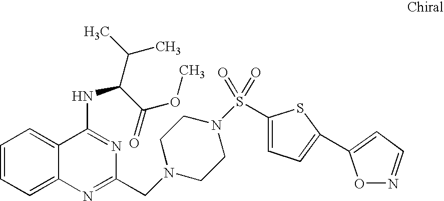 |
| 19-42 | 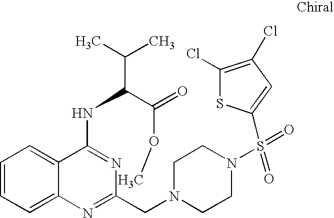 | 19-49 | 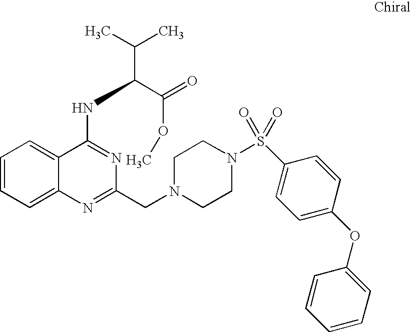 |
| 19-43 | 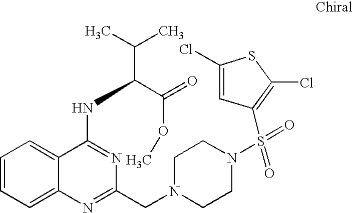 | 19-50 | 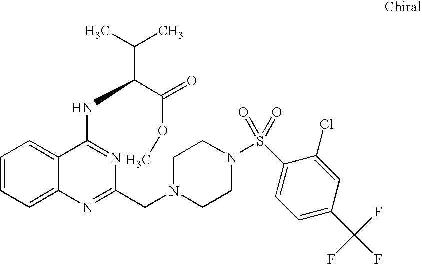 |
| 19-44 | 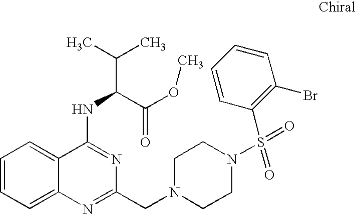 | 19-51 | 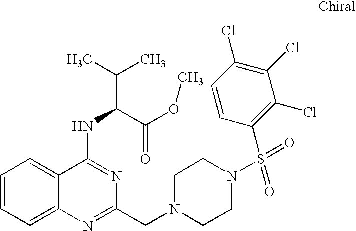 |
| 19-45 | 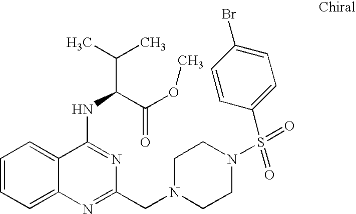 | 19-52 | 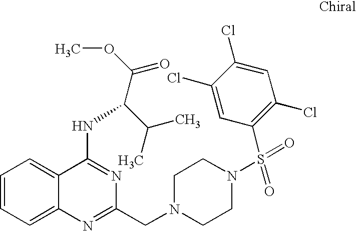 |
| 19-46 | 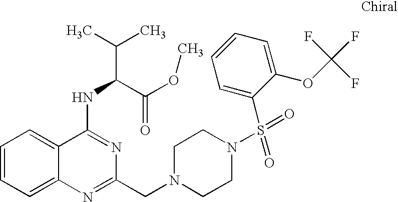 | 19-53 | 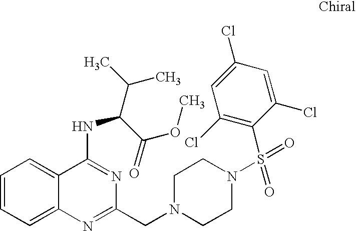 |
| 19-47 | 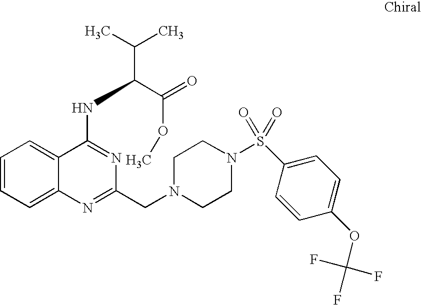 | 19-54 | 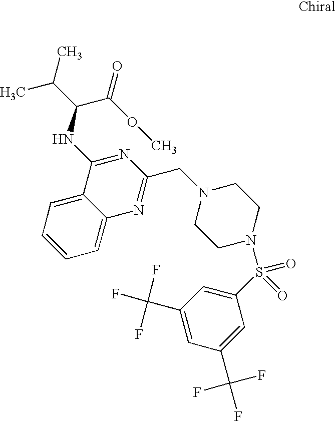 |
| 19-48 | 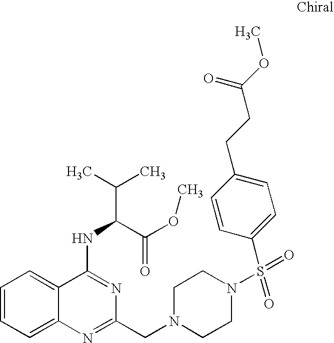 | 19-55 | 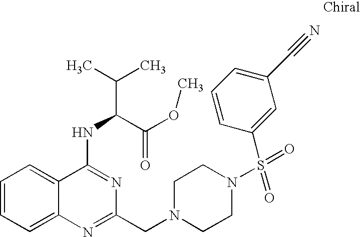 |
| 19-56 | 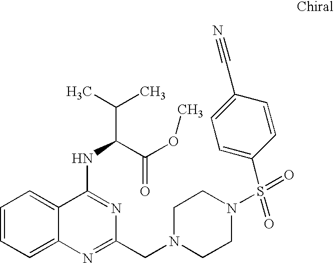 | 19-63 | 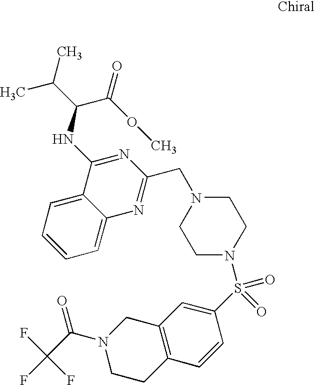 |
| 19-57 | 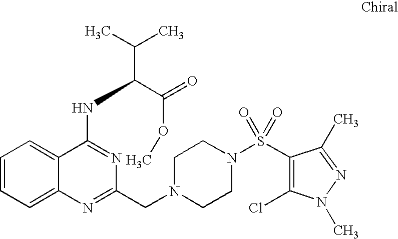 | 19-64 | 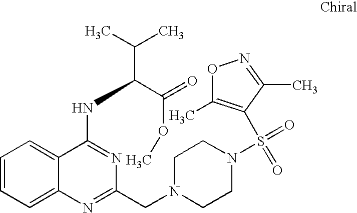 |
| 19-58 | 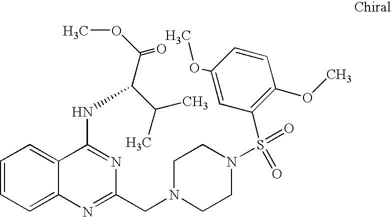 | 19-65 | 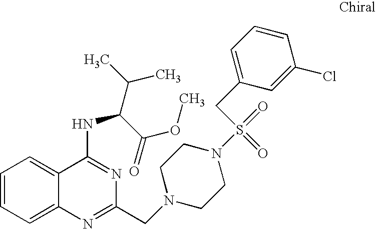 |
| 19-59 | 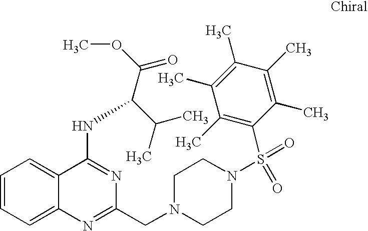 | 19-66 | 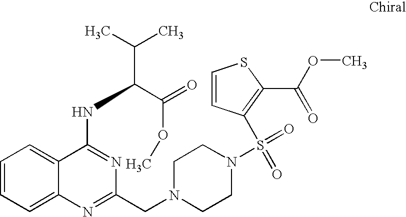 |
| 19-60 | 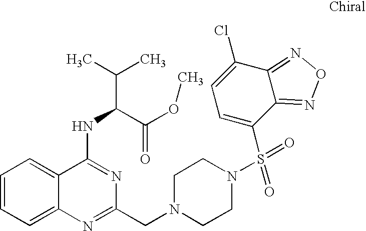 | 19-67 | 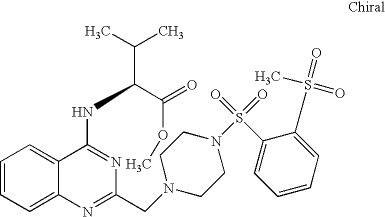 |
| 19-61 | 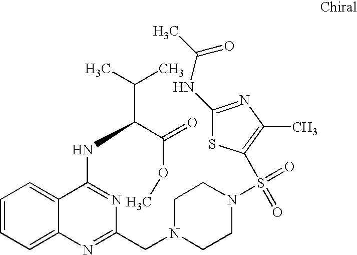 | 19-68 | 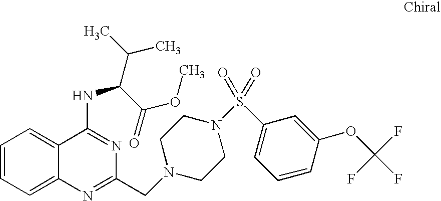 |
| 19-62 | 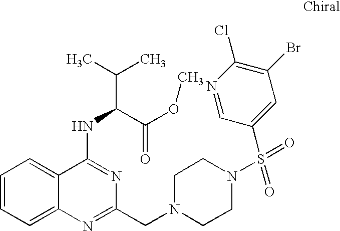 | 19-69 | 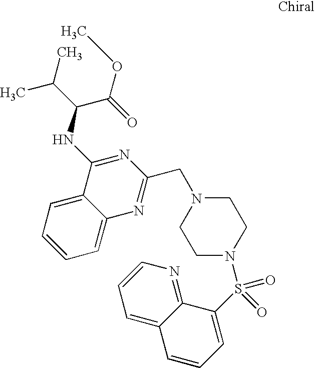 |
| 19-70 | 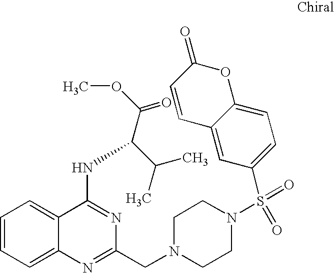 | 20-1 | 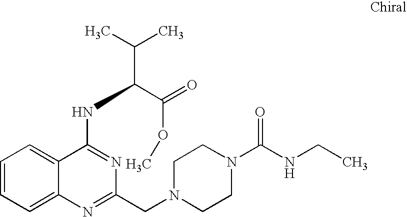 |
| 19-71 | 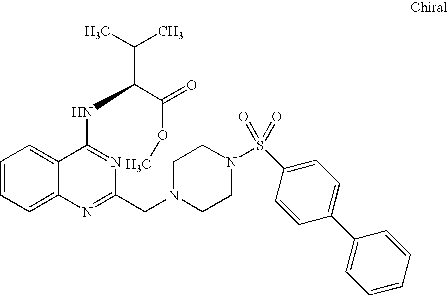 | 20-2 | 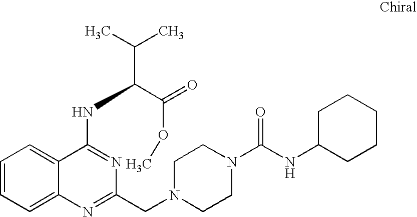 |
| 19-72 | 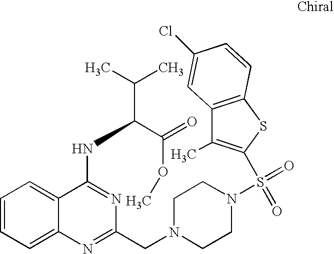 | 20-3 | 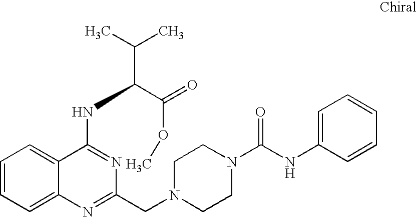 |
| 19-73 | 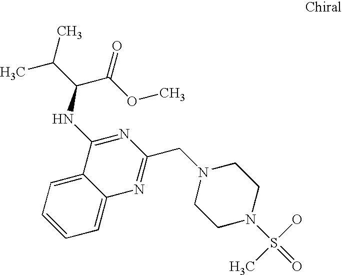 | 20-4 | 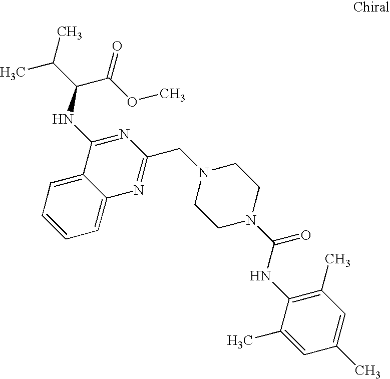 |
| 19-74 | 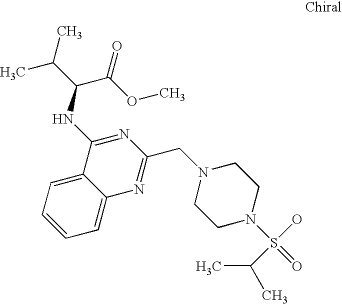 | 20-5 | 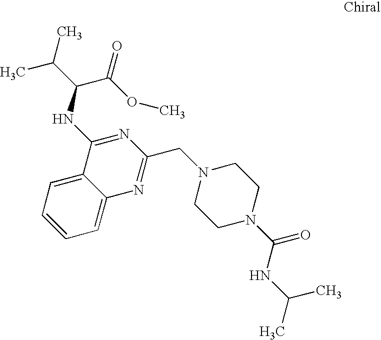 |
| 19-75 | 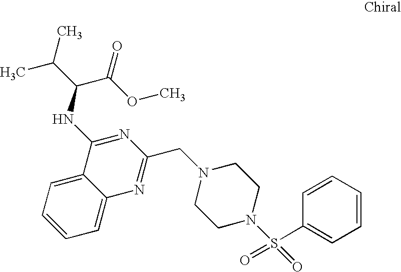 | 20-6 | 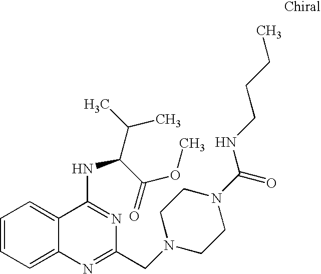 |
| 19-76 | 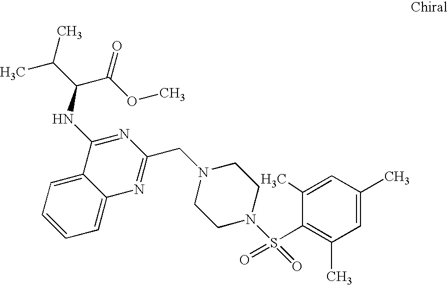 | 20-7 | 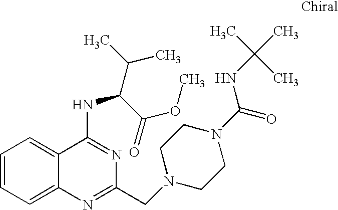 |
| 20-8 | 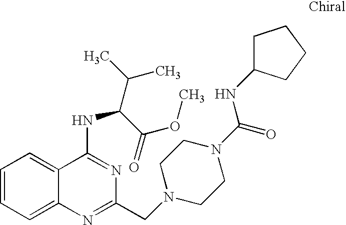 | 20-15 | 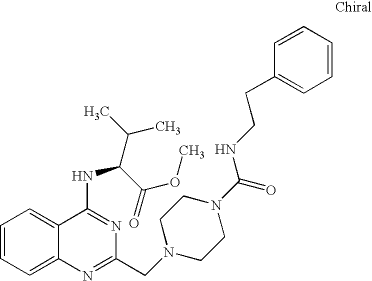 |
| 20-9 | 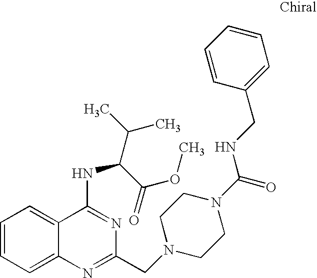 | 20-16 | 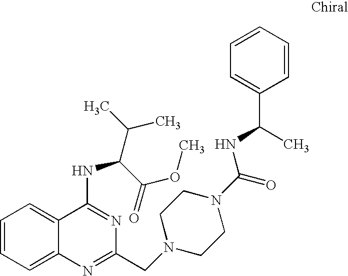 |
| 20-10 | 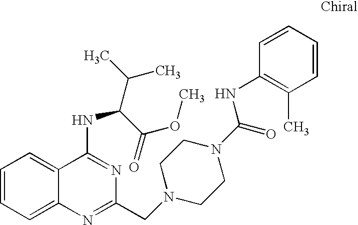 | 20-17 | 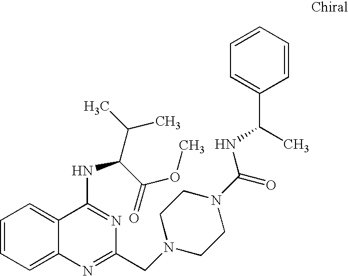 |
| 20-11 | 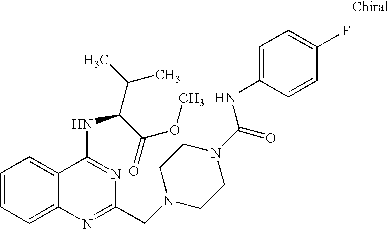 | 20-18 | 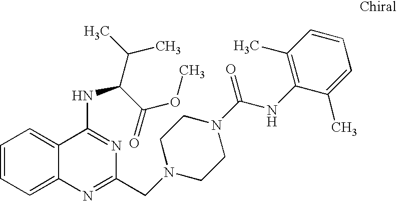 |
| 20-12 | 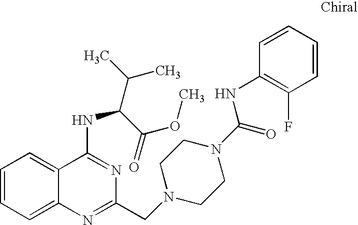 | 20-19 | 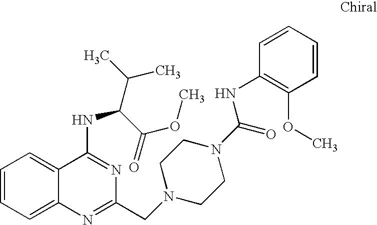 |
| 20-13 | 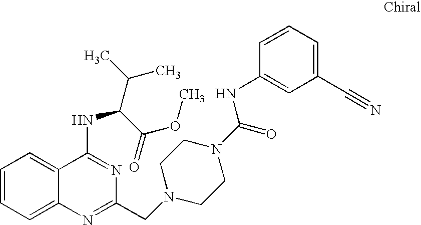 | 20-20 | 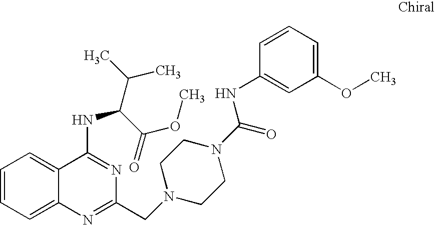 |
| 20-14 | 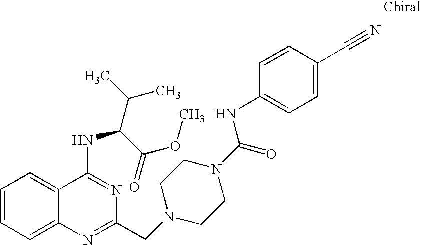 | 20-21 | 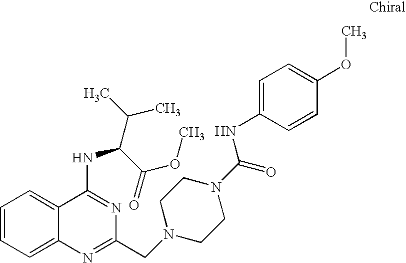 |
| 20-22 | 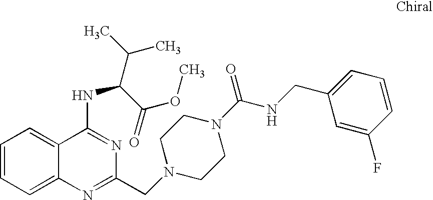 | 20-29 | 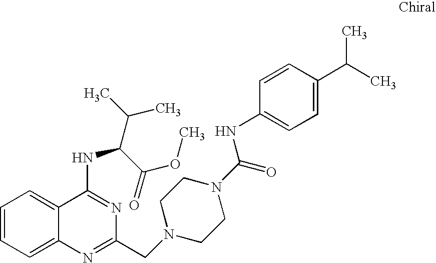 |
| 20-23 | 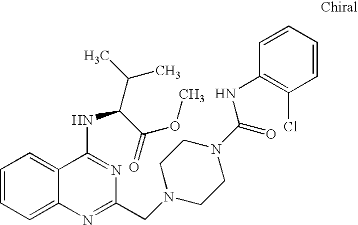 | 20-30 | 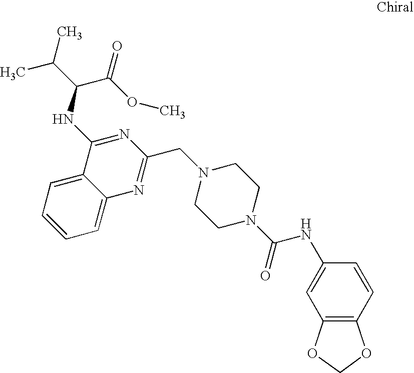 |
| 20-24 | 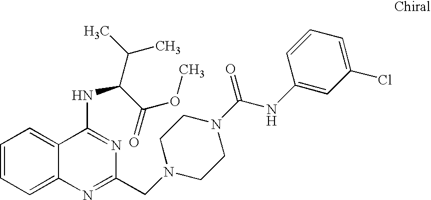 | 20-31 | 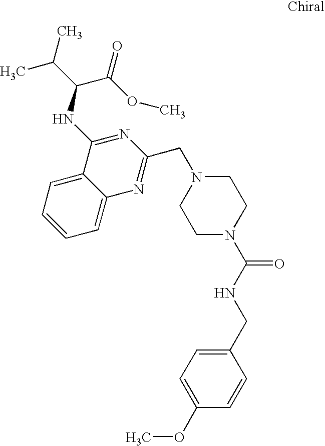 |
| 20-25 | 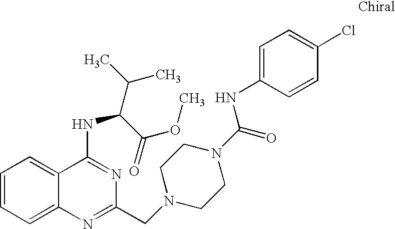 | 20-32 | 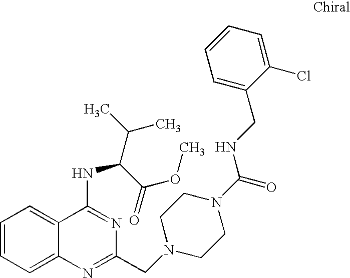 |
| 20-26 | 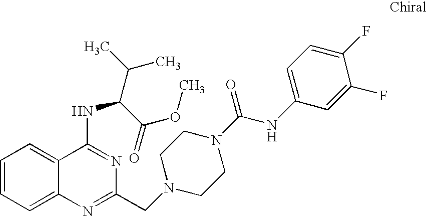 | 20-33 | 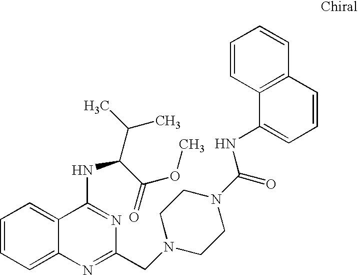 |
| 20-27 | 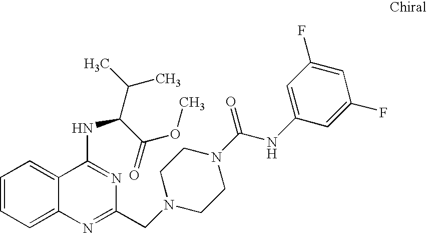 | 20-34 | 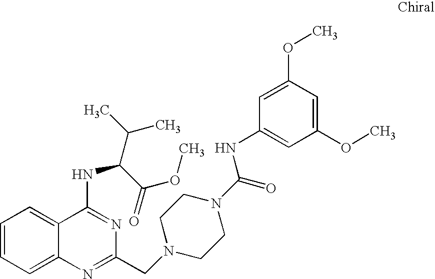 |
| 20-28 | 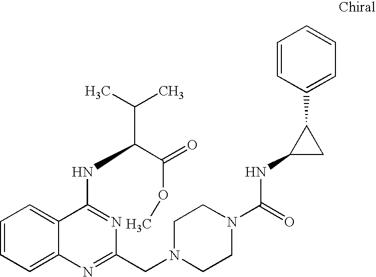 | 20-35 | 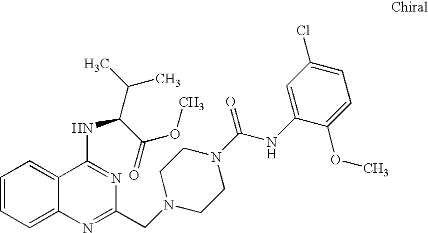 |
| 20-36 | 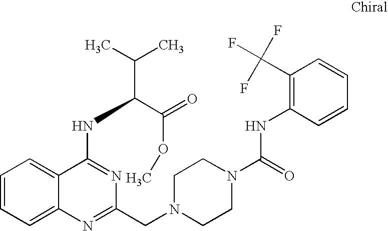 | 20-43 | 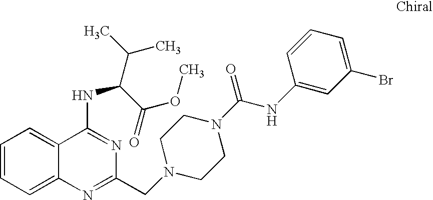 |
| 20-37 | 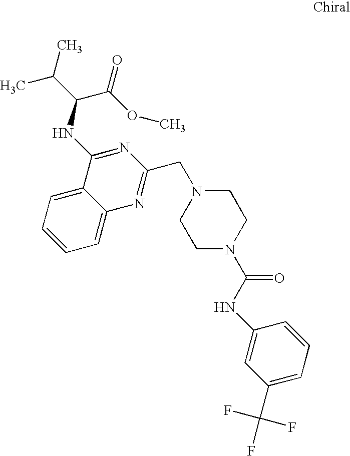 | 20-44 | 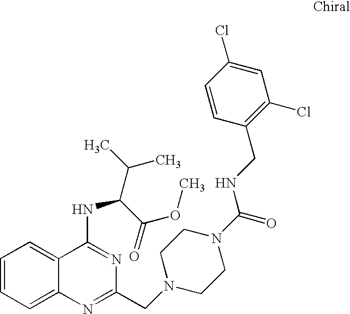 |
| 20-38 | 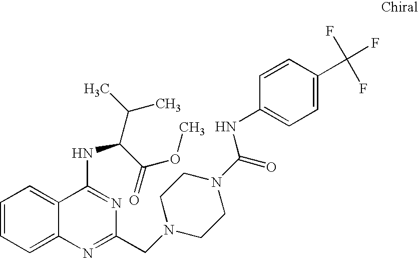 | 20-45 | 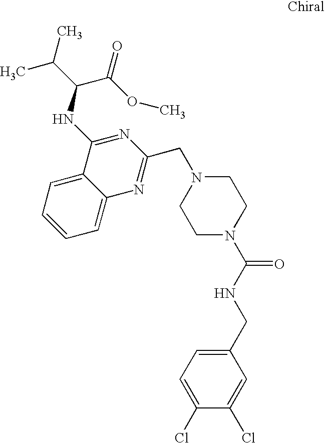 |
| 20-39 | 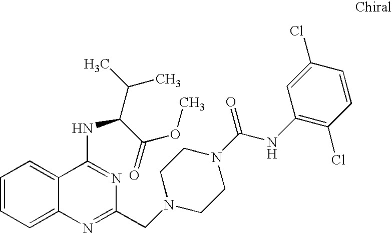 | 20-46 | 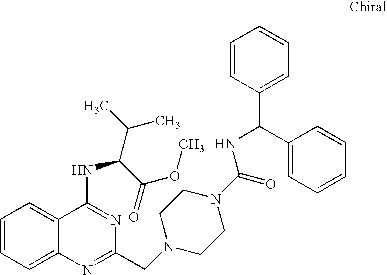 |
| 20-40 | 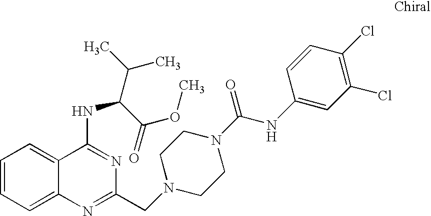 | 20-47 | 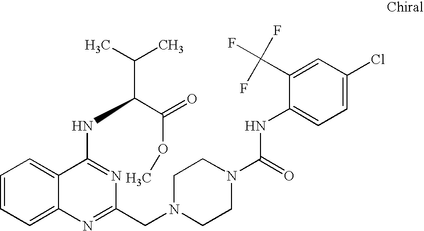 |
| 20-41 | 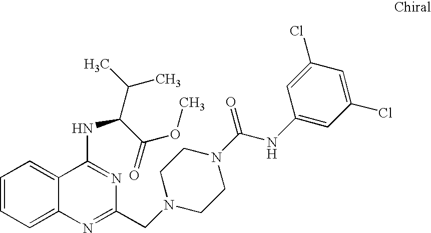 | 20-48 | 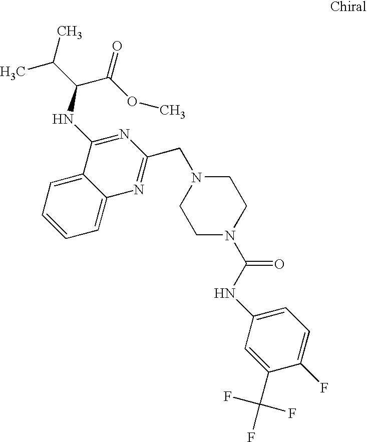 |
| 20-42 | 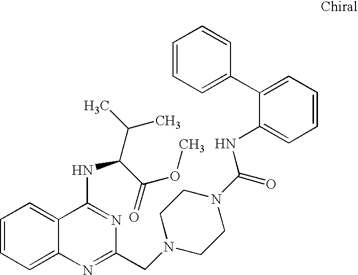 | 20-49 | 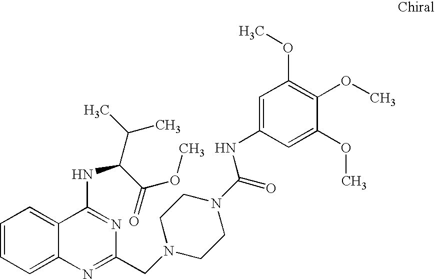 |
| 20-50 | 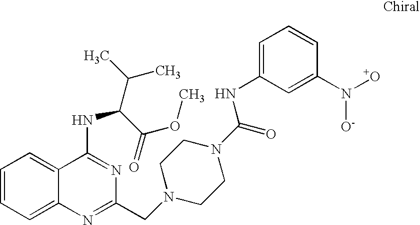 | 21-7 | 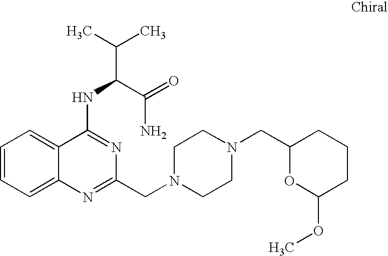 |
| 21-1 | 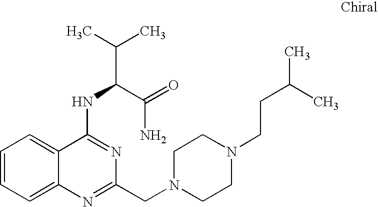 | 21-8 | 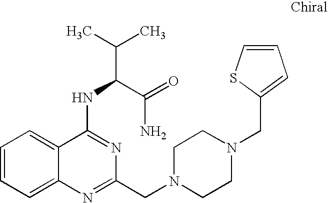 |
| 21-2 | 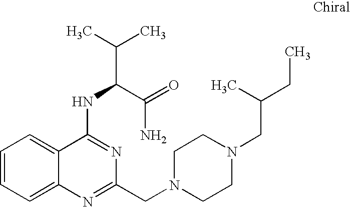 | 21-9 | 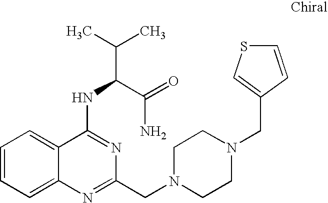 |
| 21-3 | 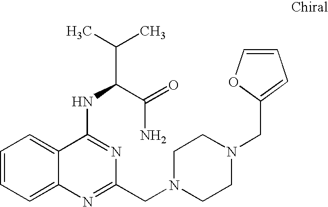 | 21-10 | 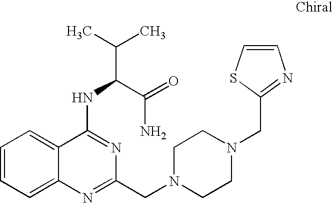 |
| 21-4 | 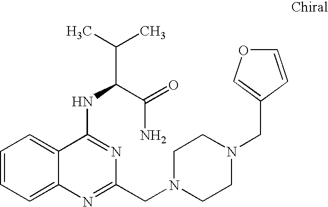 | 21-11 | 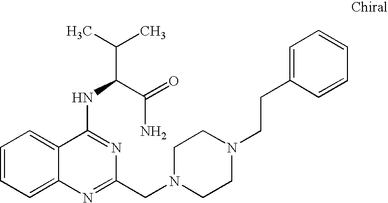 |
| 21-5 | 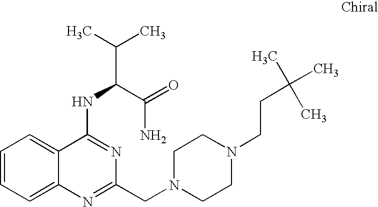 | 21-12 | 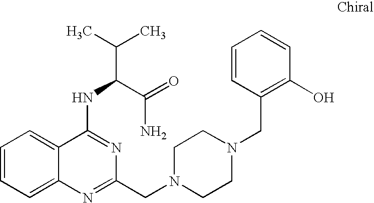 |
| 21-6 | 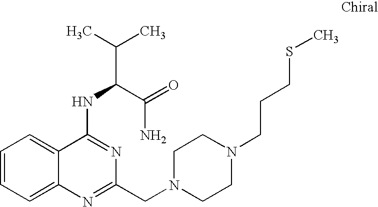 | 21-13 | 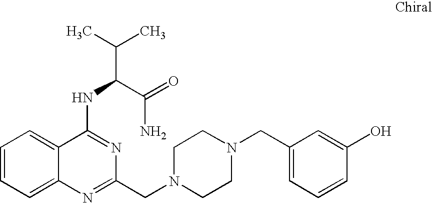 |
| 21-14 | 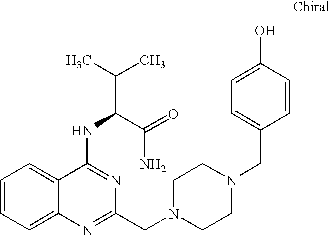 | 21-21 | 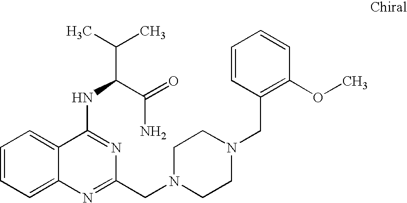 |
| 21-15 | 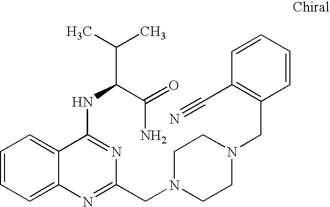 | 21-22 | 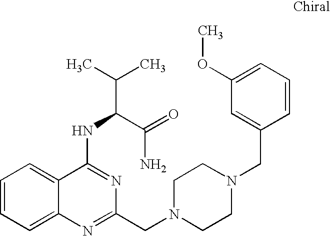 |
| 21-16 | 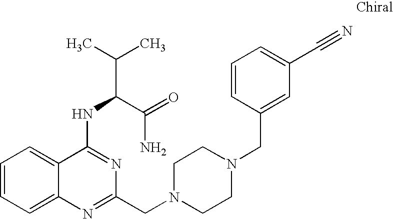 | 21-23 | 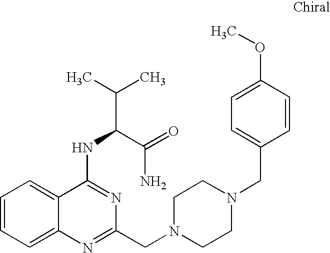 |
| 21-17 | 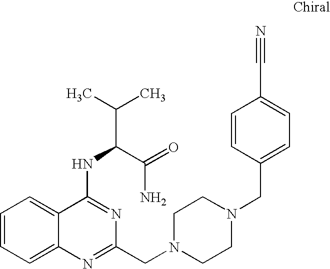 | 21-24 | 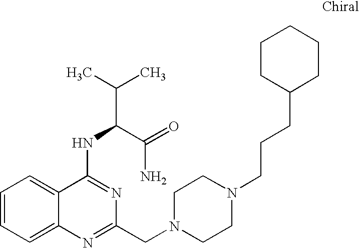 |
| 21-18 | 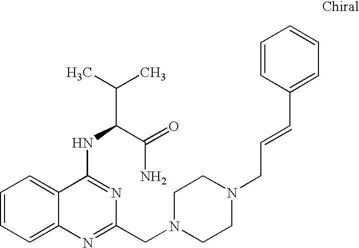 | 21-25 | 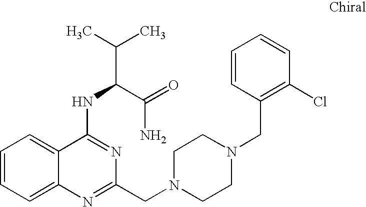 |
| 21-19 | 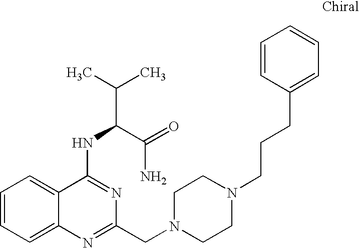 | 21-26 | 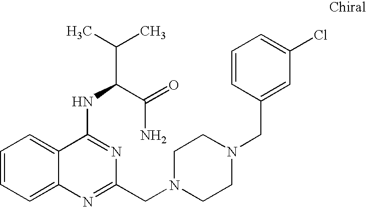 |
| 21-20 | 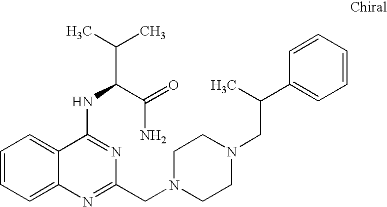 | 21-27 | 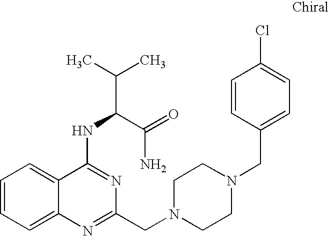 |
| 21-28 | 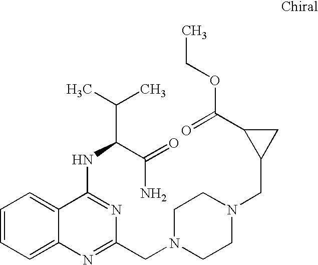 | 21-35 | 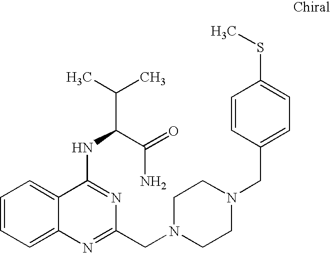 |
| 21-29 | 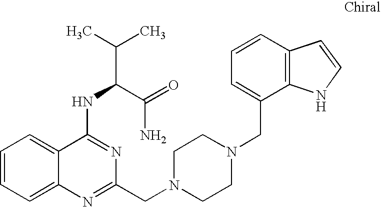 | 21-36 | 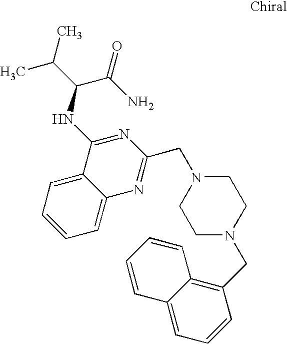 |
| 21-30 | 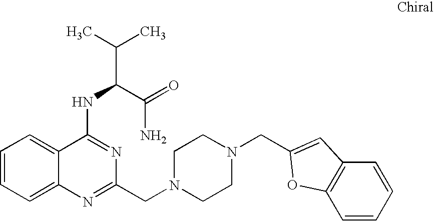 | 21-37 | 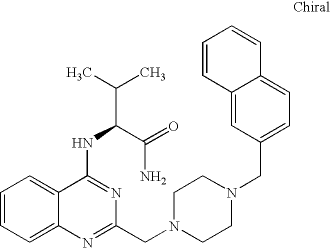 |
| 21-31 | 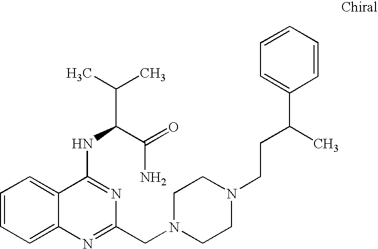 | 21-38 | 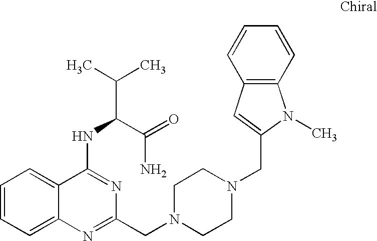 |
| 21-32 | 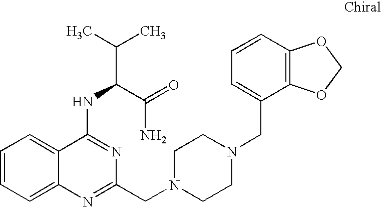 | 21-39 | 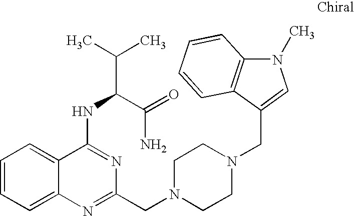 |
| 21-33 | 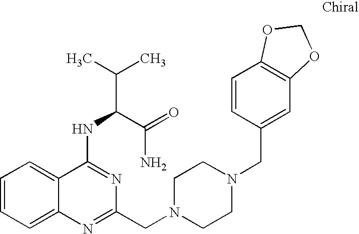 | 21-40 | 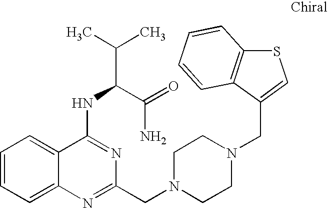 |
| 21-34 | 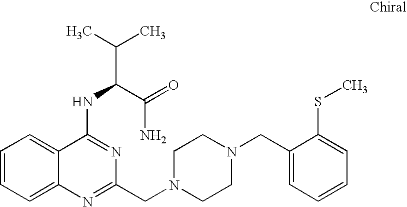 | 21-41 | 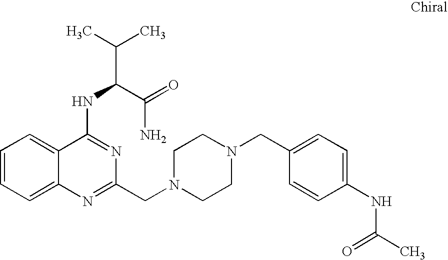 |
| 21-42 | 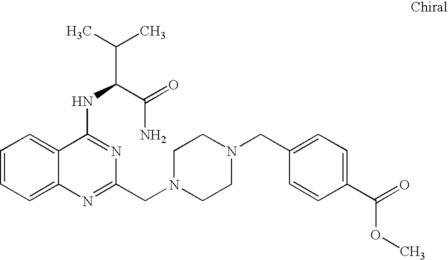 | 21-45 | 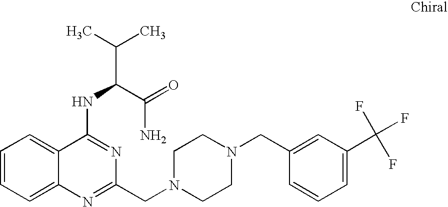 |
| 21-43 | 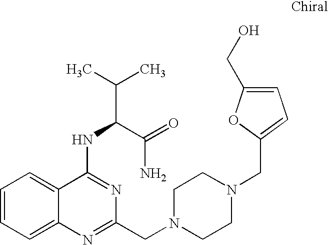 | 21-46 | 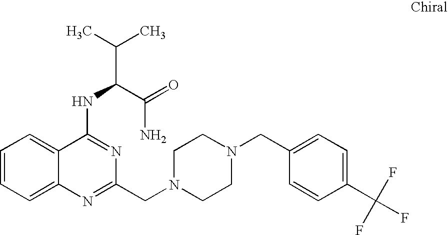 |
| 21-44 | 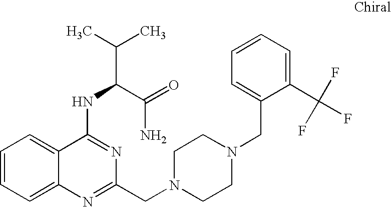 | 21-47 | 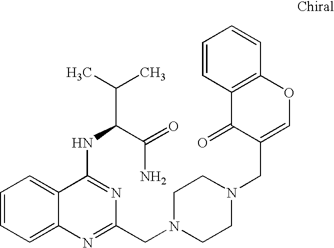 |
| 21-52 | 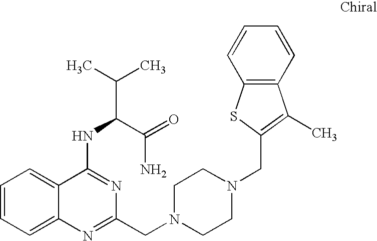 | 21-48 | 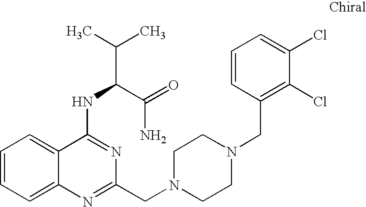 |
| 21-53 | 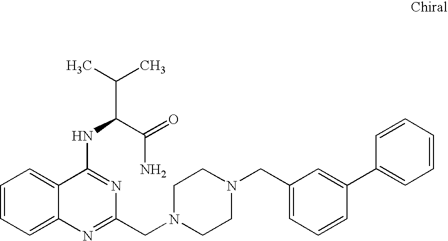 | 21-49 | 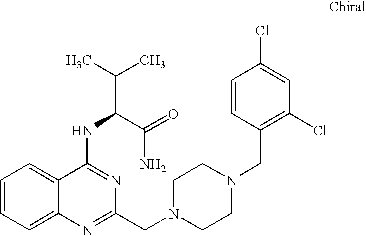 |
| 21-54 | 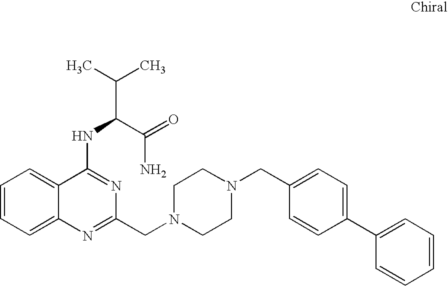 | 21-50 | 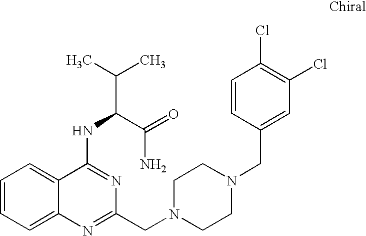 |
| 21-55 | 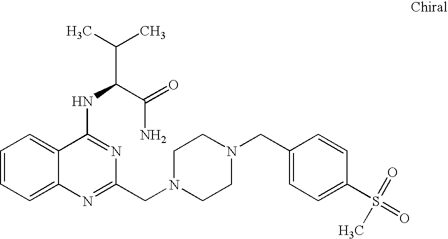 | 21-51 | 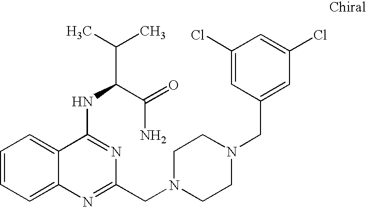 |
| 21-56 | 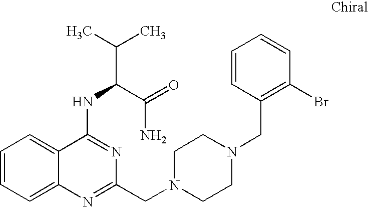 | 21-63 | 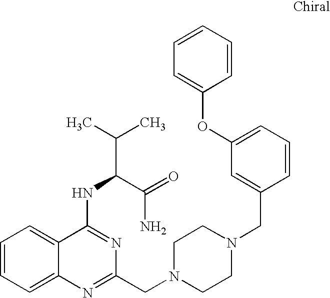 |
| 21-57 | 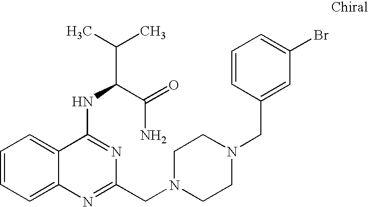 | 21-64 | 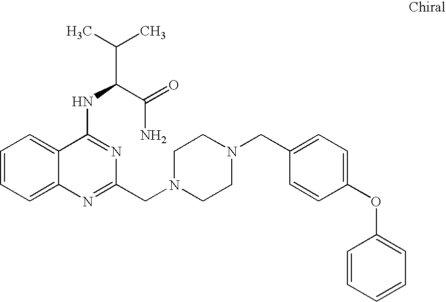 |
| 21-58 | 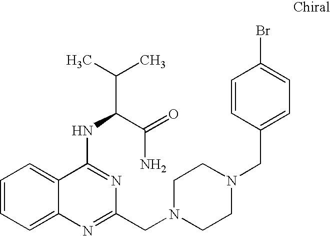 | 21-65 | 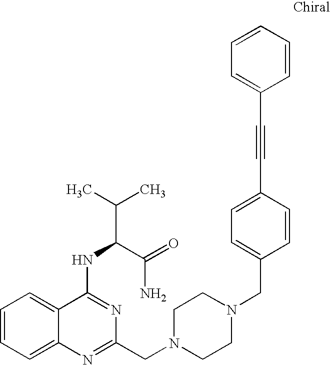 |
| 21-59 | 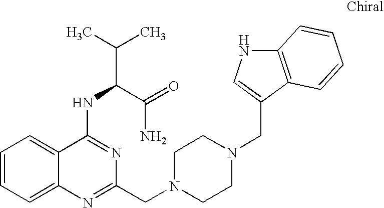 | 21-66 | 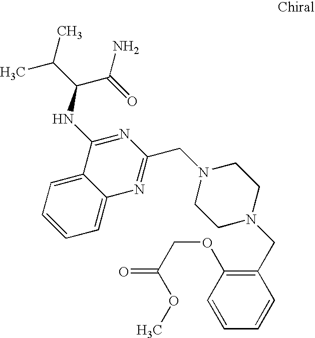 |
| 21-60 | 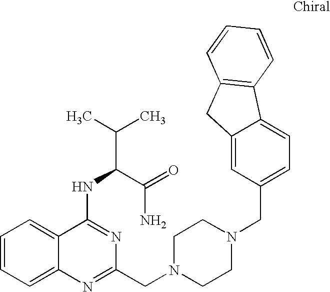 | 21-67 | 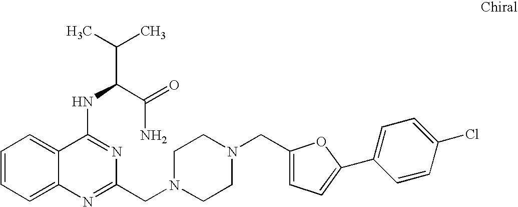 |
| 21-61 | 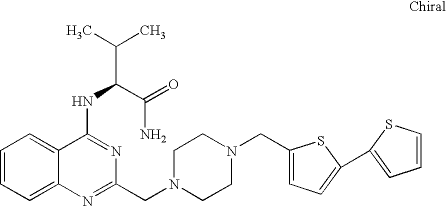 | 21-68 | 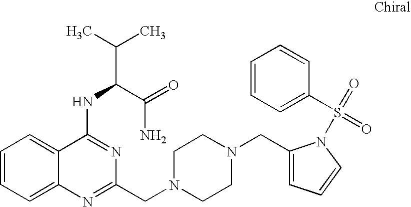 |
| 21-62 | 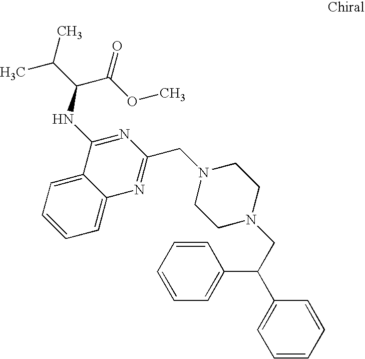 | 21-69 | 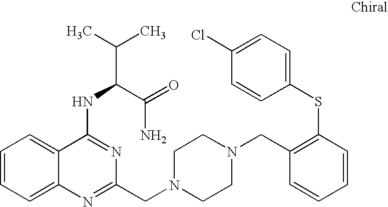 |
| 21-70 | 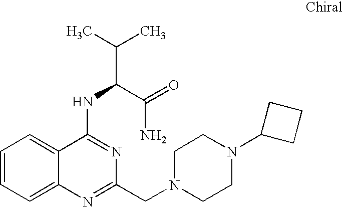 | 21-77 | 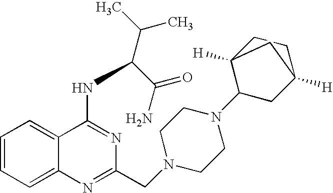 |
| 21-71 | 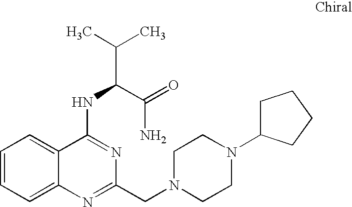 | 21-78 | 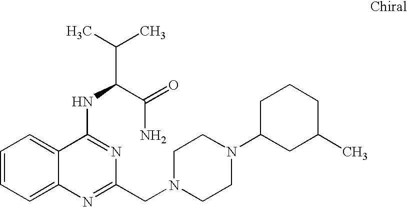 |
| 21-72 | 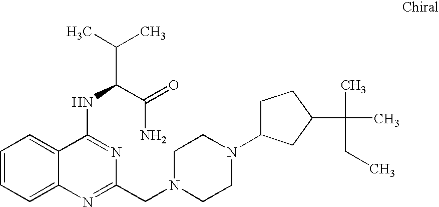 | 21-79 | 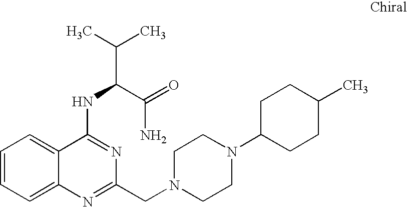 |
| 21-73 | 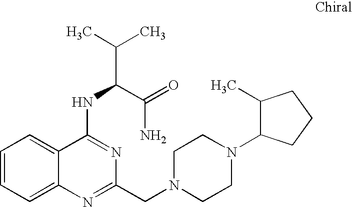 | 21-80 | 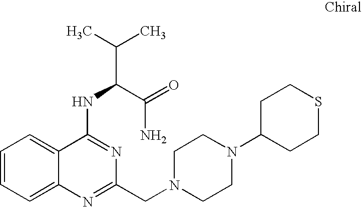 |
| 21-74 | 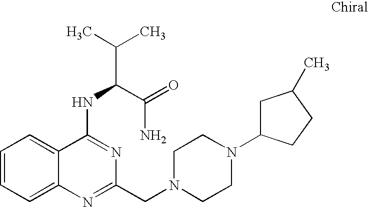 | 21-81 | 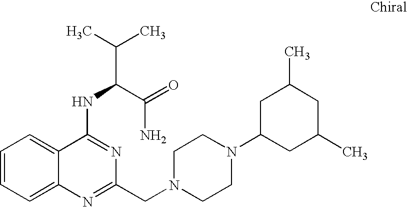 |
| 21-75 | 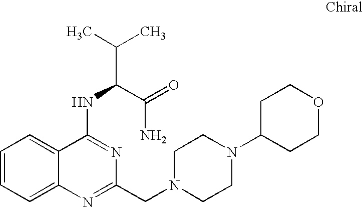 | 21-82 | 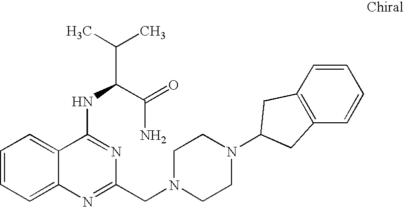 |
| 21-76 | 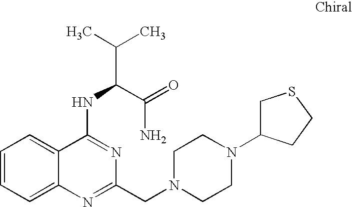 | 21-83 | 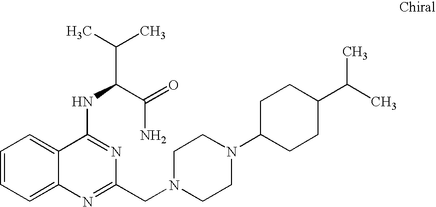 |
| 21-84 | 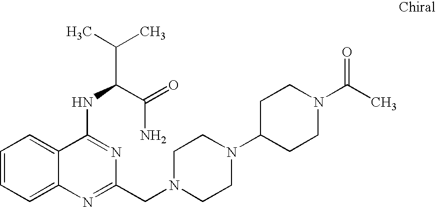 | 21-91 | 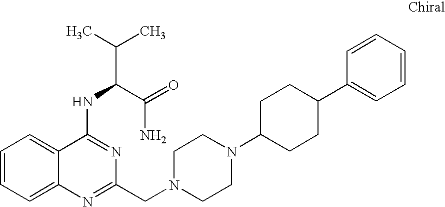 |
| 21-85 | 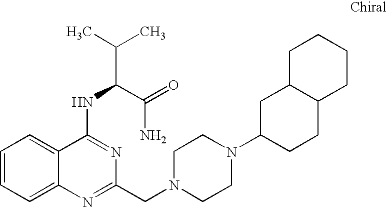 | 21-92 | 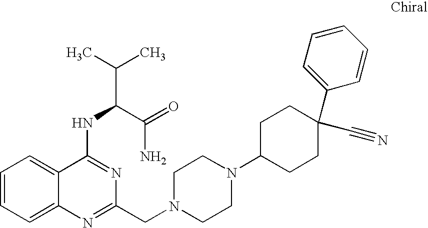 |
| 21-86 | 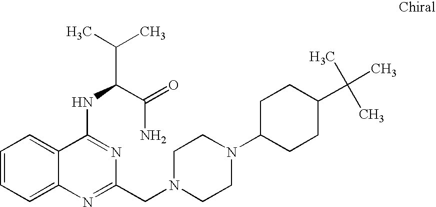 | 21-93 | 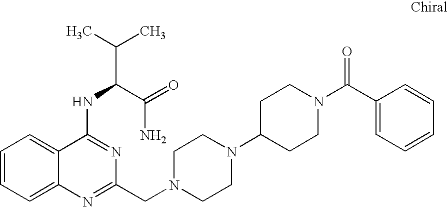 |
| 21-87 | 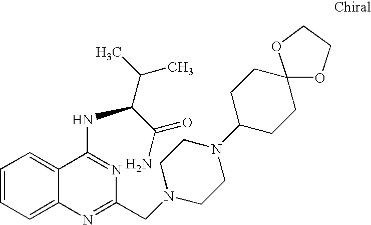 | 21-94 | 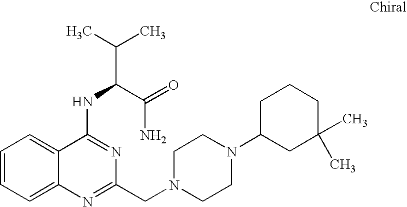 |
| 21-88 | 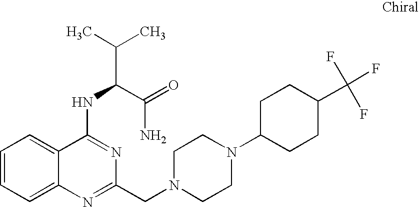 | 21-95 | 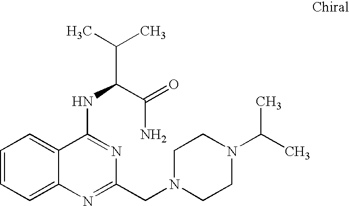 |
| 21-89 | 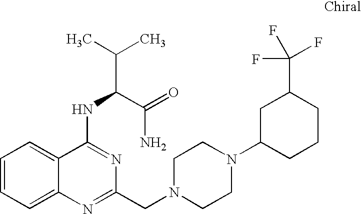 | 21-96 | 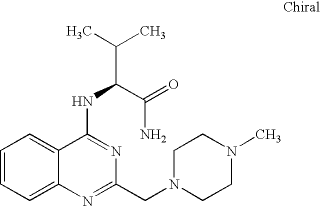 |
| 21-90 | 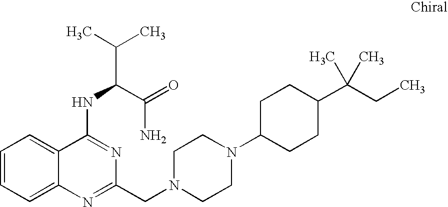 | 21-97 | 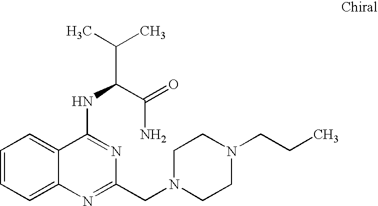 |
| 22-1 | 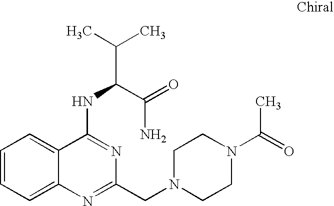 | 22-8 | 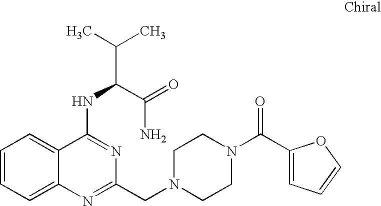 |
| 22-2 | 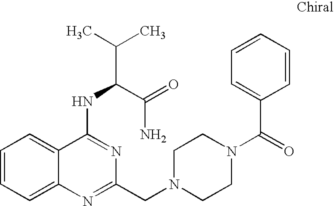 | 22-9 | 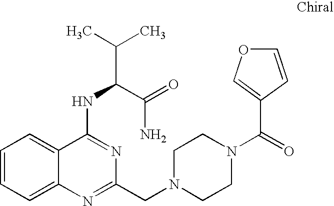 |
| 22-3 | 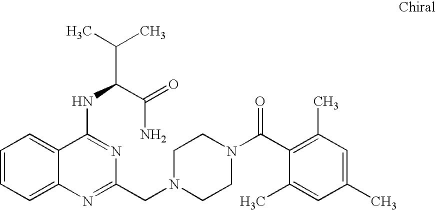 | 22-10 | 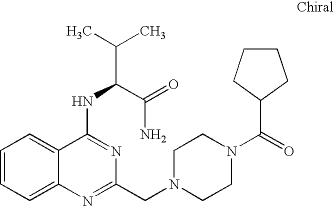 |
| 22-4 | 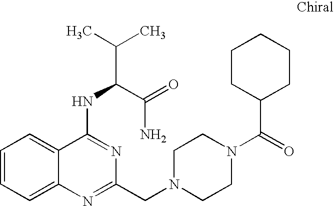 | 22-11 | 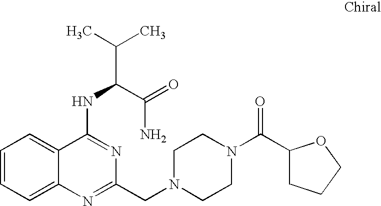 |
| 22-5 | 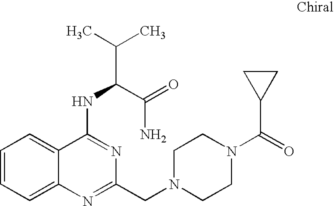 | 22-12 | 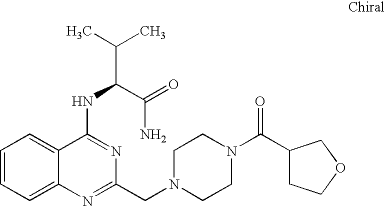 |
| 22-6 | 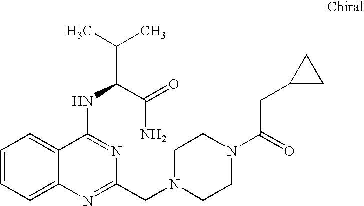 | 22-13 | 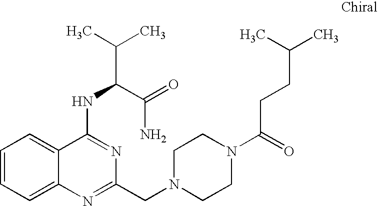 |
| 22-7 | 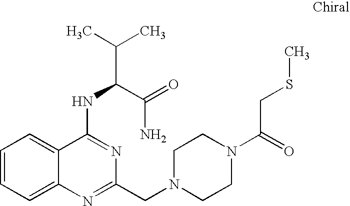 | 22-14 | 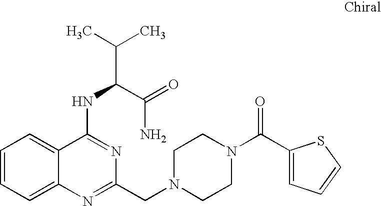 |
| 22-15 | 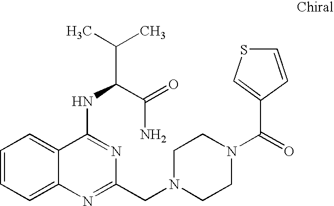 | 22-22 | 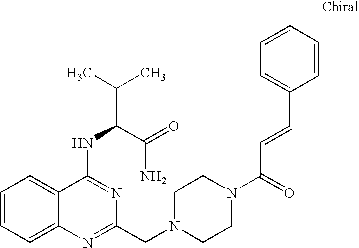 |
| 22-16 | 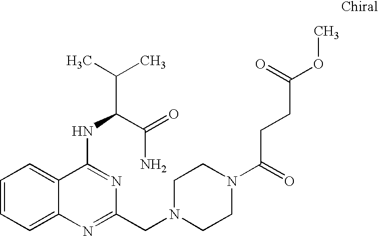 | 22-23 | 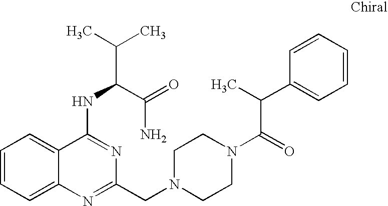 |
| 22-17 | 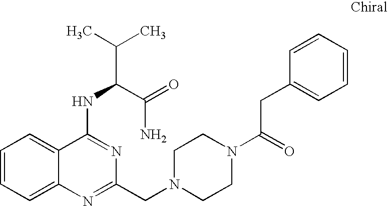 | 22-24 | 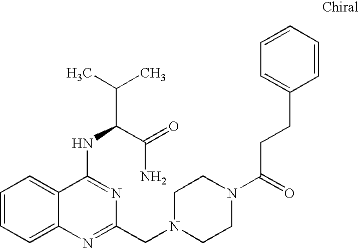 |
| 22-18 | 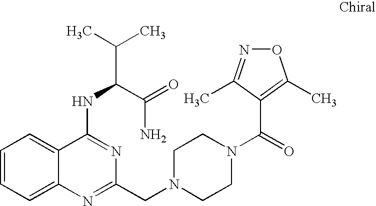 | 22-25 | 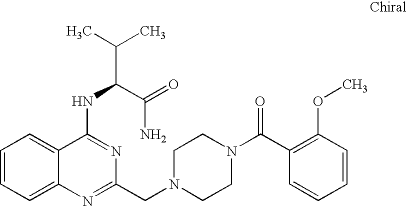 |
| 22-19 | 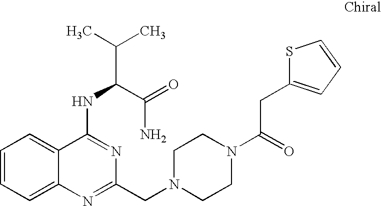 | 22-26 | 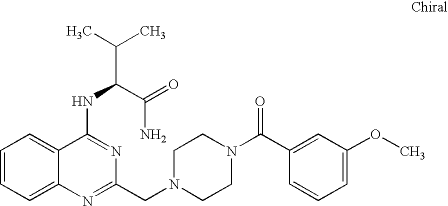 |
| 22-20 | 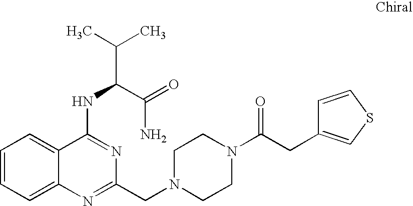 | 22-27 | 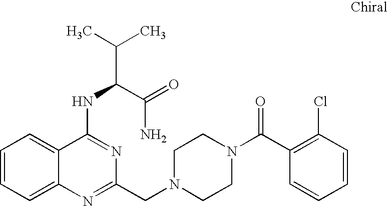 |
| 22-21 | 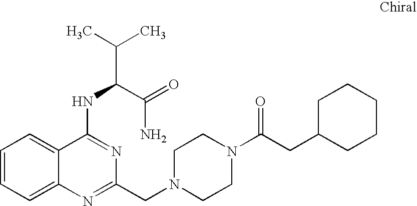 | 22-28 | 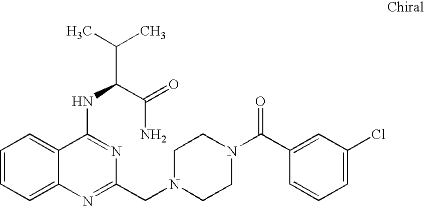 |
| 22-29 | 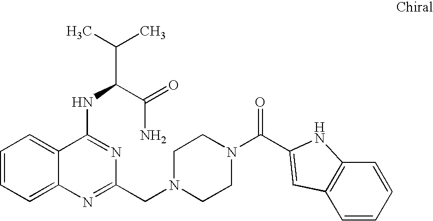 | 22-36 | 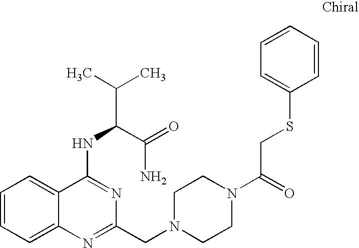 |
| 22-30 | 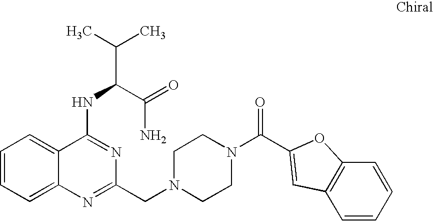 | 22-37 | 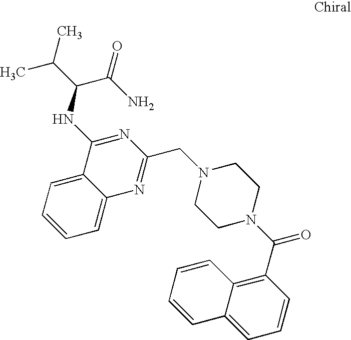 |
| 22-31 | 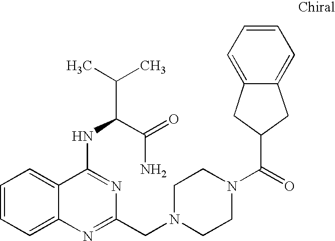 | 22-38 | 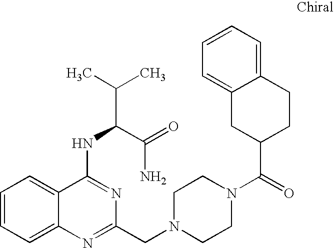 |
| 22-32 | 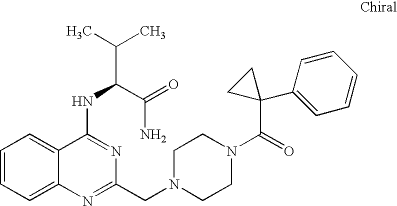 | 22-39 | 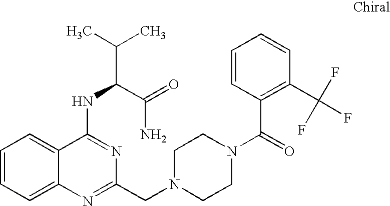 |
| 22-33 | 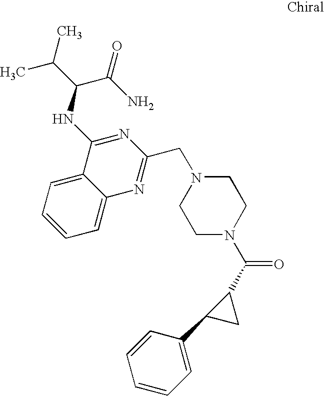 | 22-40 | 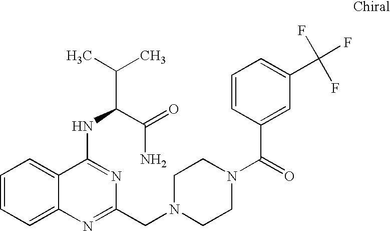 |
| 22-34 | 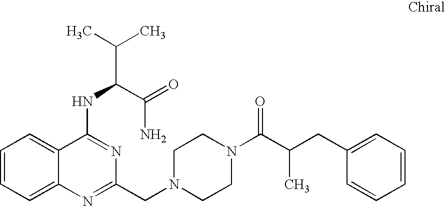 | 22-41 | 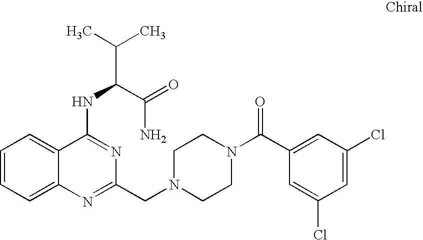 |
| 22-35 | 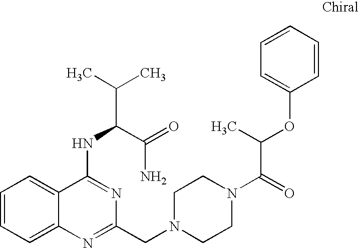 | 22-42 | 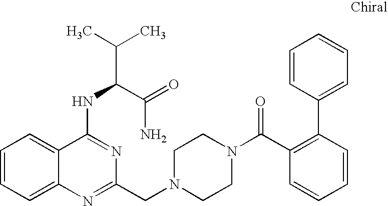 |
| 22-43 | 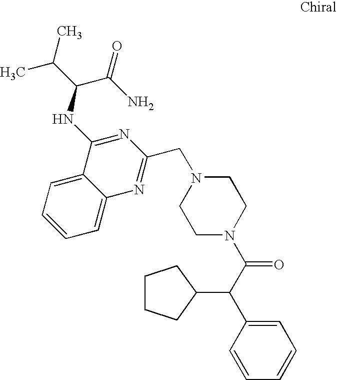 | 22-50 | 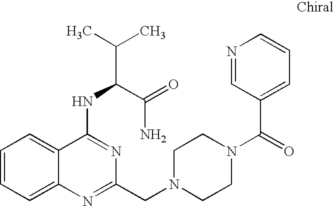 |
| 22-44 | 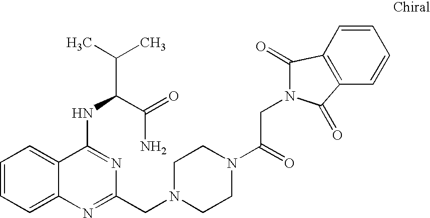 | 22-51 | 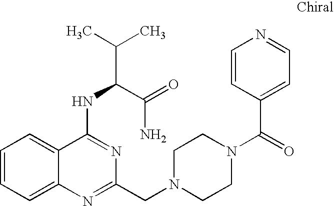 |
| 22-45 | 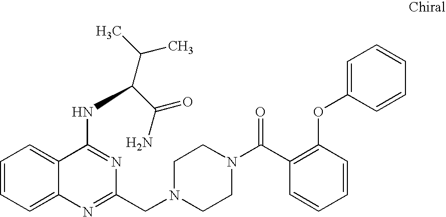 | 22-52 | 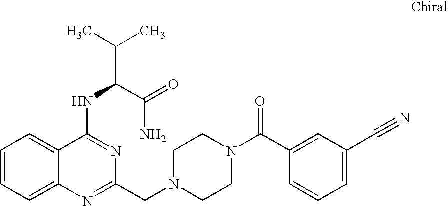 |
| 22-46 | 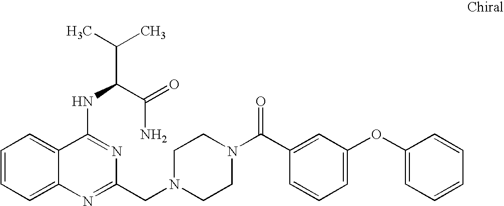 | 22-53 | 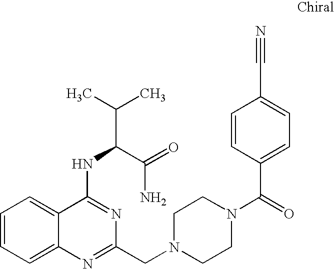 |
| 22-47 | 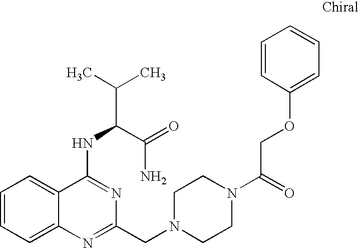 | 22-54 | 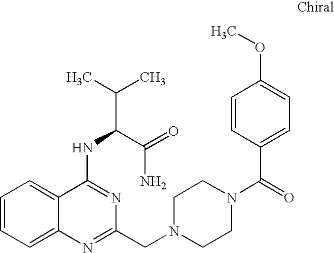 |
| 22-48 | 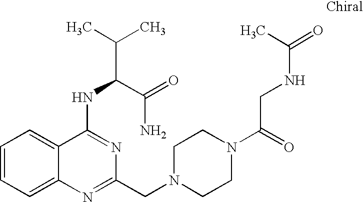 | 22-55 | 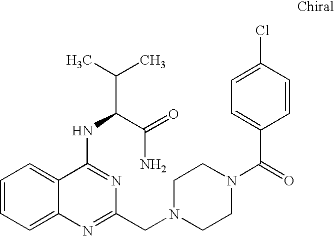 |
| 22-49 | 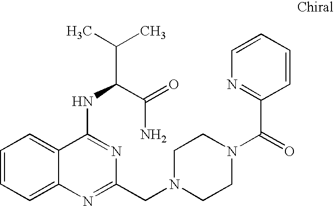 | 22-56 | 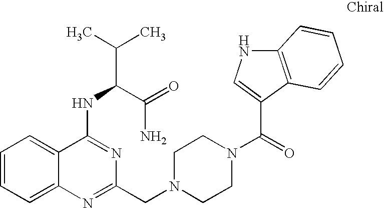 |
| 22-57 | 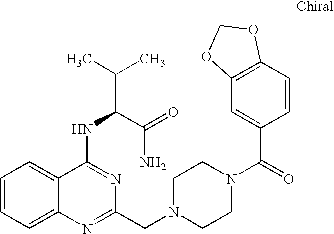 | 22-64 | 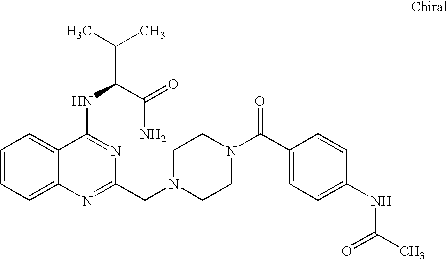 |
| 22-58 | 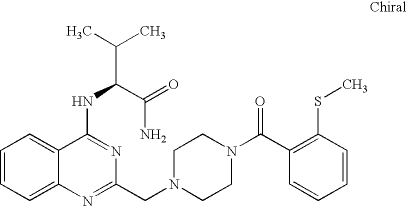 | 22-65 | 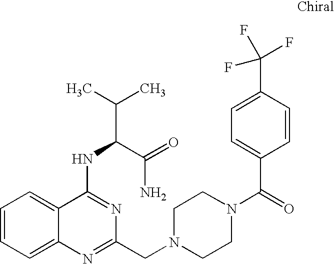 |
| 22-59 | 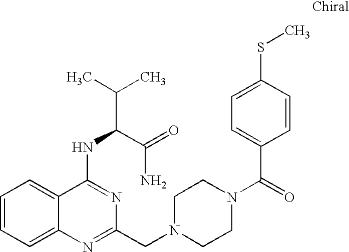 | 22-66 | 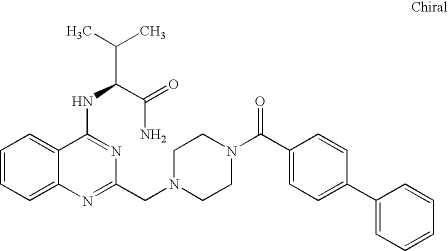 |
| 22-60 | 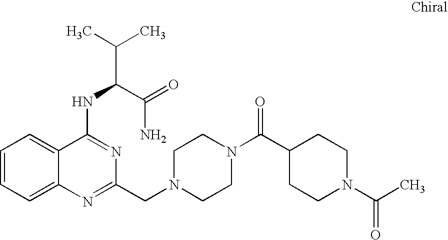 | 22-67 | 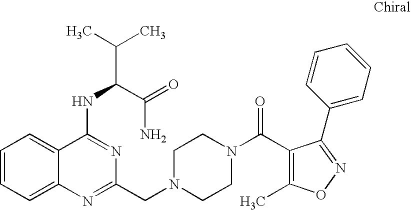 |
| 22-61 | 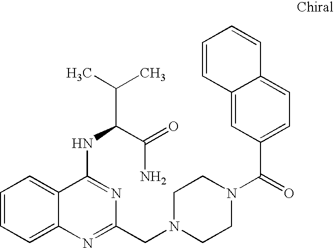 | 22-68 | 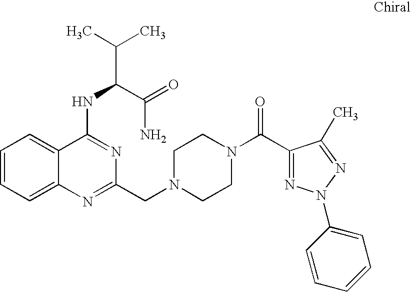 |
| 22-62 | 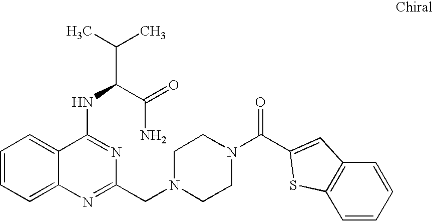 | 22-69 | 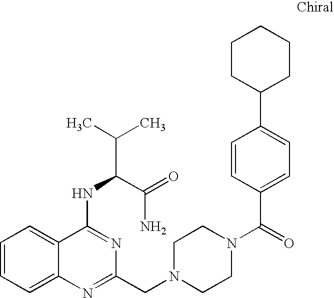 |
| 22-63 | 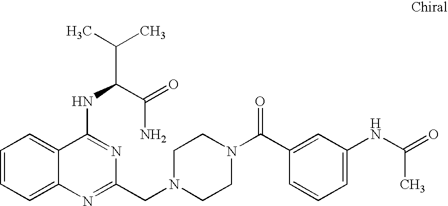 | 22-70 | 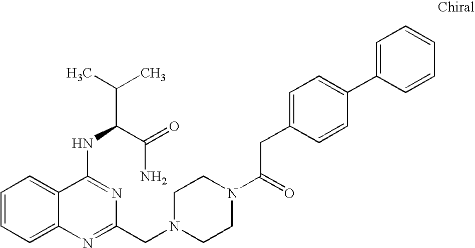 |
| 22-71 | 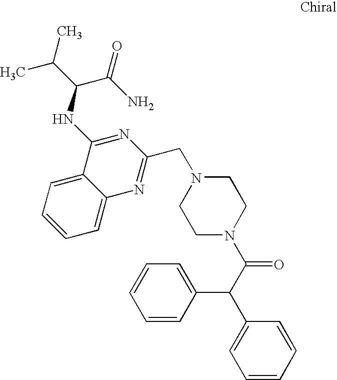 | 23-7 | 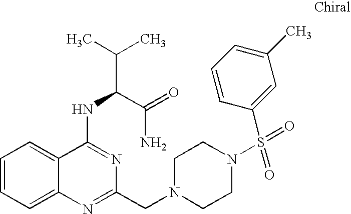 |
| 23-1 | 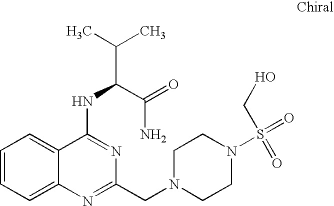 | 23-8 | 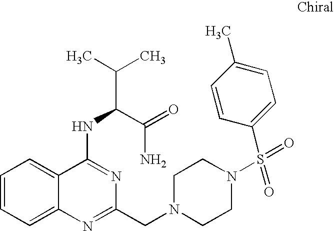 |
| 23-2 | 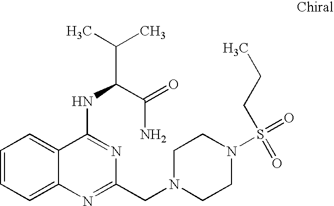 | 23-9 | 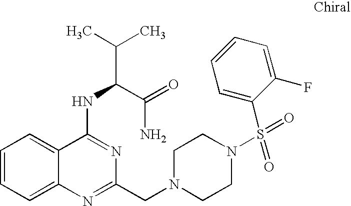 |
| 23-3 | 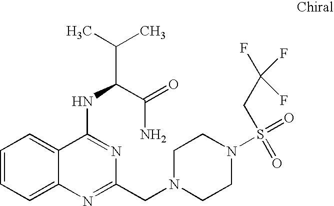 | 23-10 | 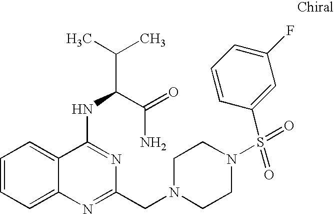 |
| 23-4 | 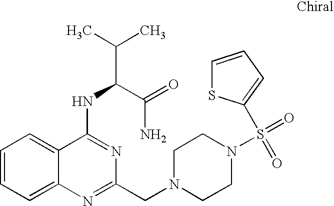 | 23-11 | 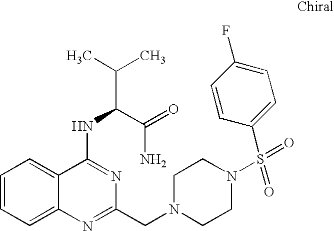 |
| 23-5 | 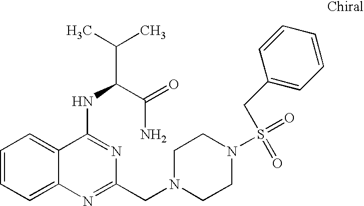 | 23-12 | 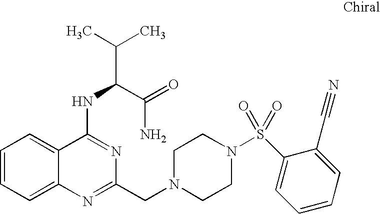 |
| 23-6 | 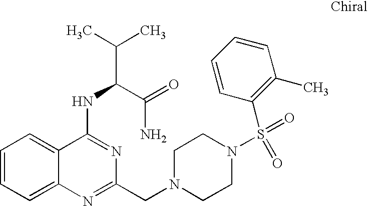 | 23-13 | 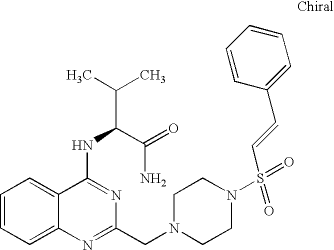 |
| 23-14 | 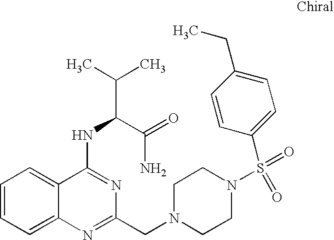 | 23-21 | 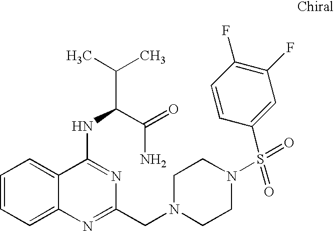 |
| 23-15 | 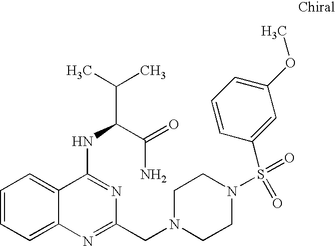 | 23-22 | 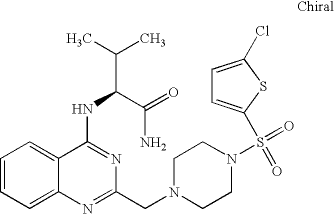 |
| 23-16 | 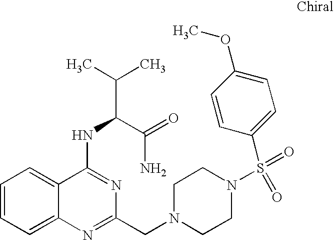 | 23-23 | 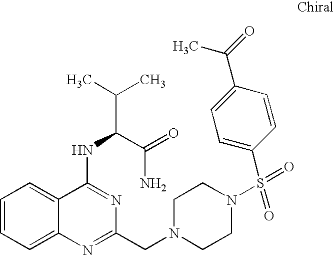 |
| 23-17 | 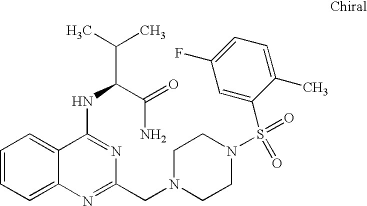 | 23-24 | 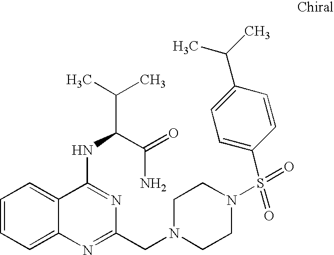 |
| 23-18 | 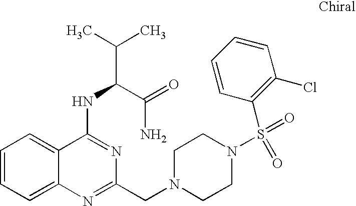 | 23-25 | 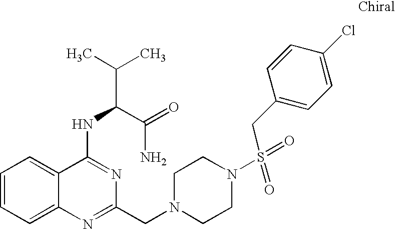 |
| 23-19 | 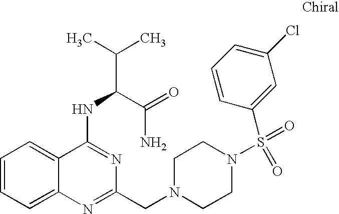 | 23-26 | 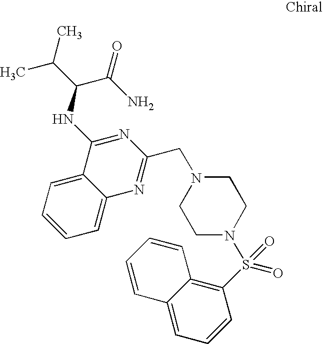 |
| 23-20 | 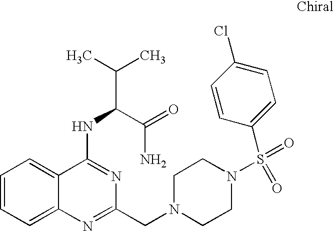 | 23-27 | 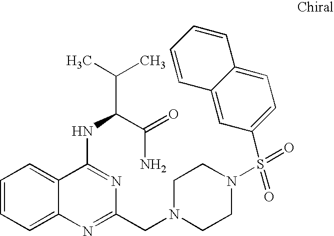 |
| 23-28 | 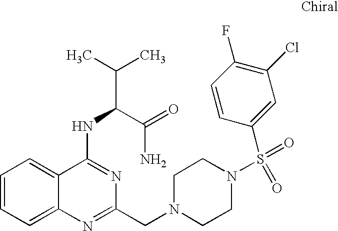 | 23-35 | 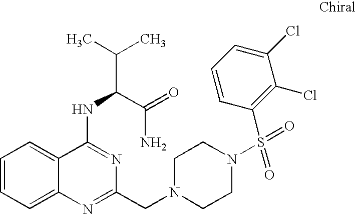 |
| 23-29 | 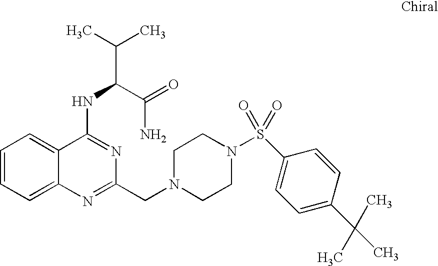 | 23-36 | 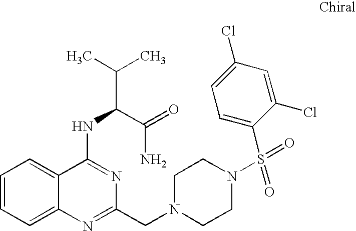 |
| 23-30 | 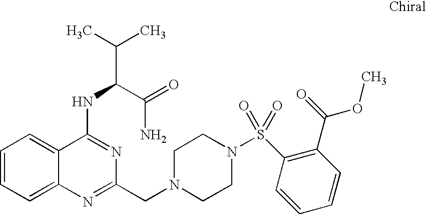 | 23-37 | 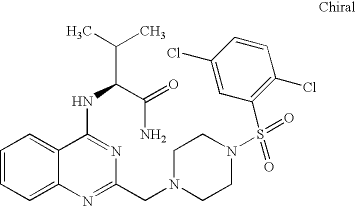 |
| 23-31 | 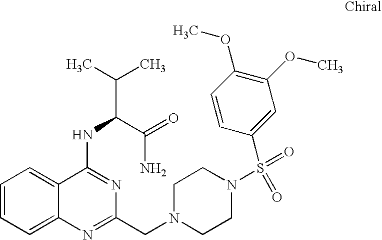 | 23-38 | 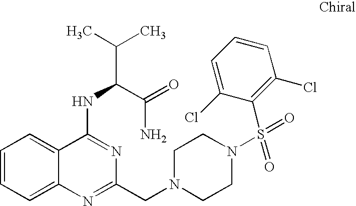 |
| 23-32 | 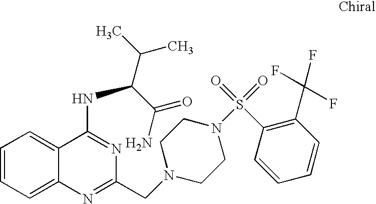 | 23-39 | 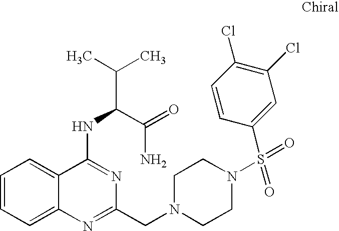 |
| 23-33 | 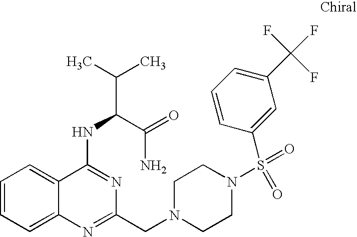 | 23-40 | 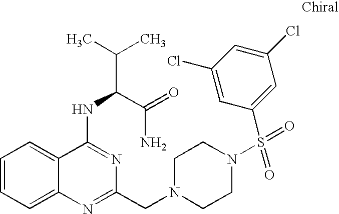 |
| 23-34 | 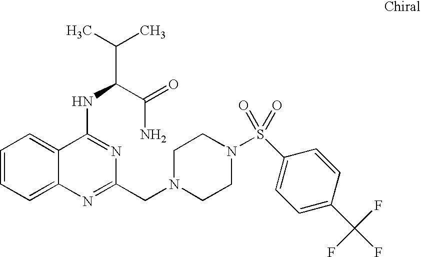 | 23-41 | 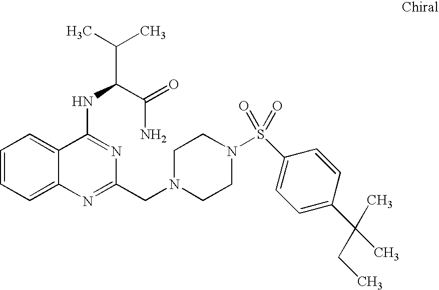 |
| 23-42 | 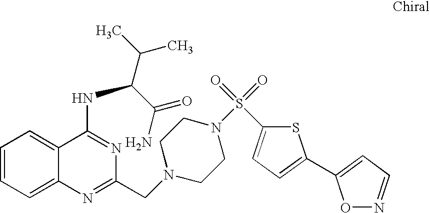 | 23-49 | 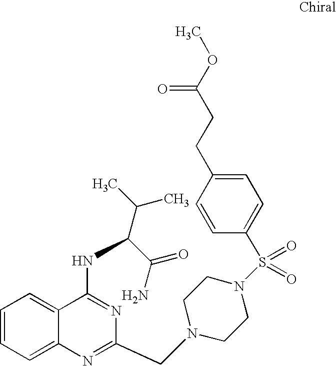 |
| 23-43 | 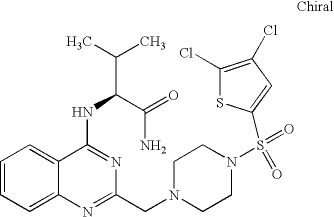 | 23-50 | 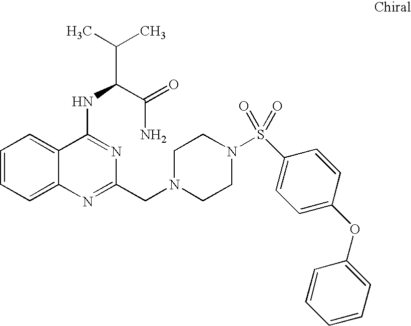 |
| 23-44 | 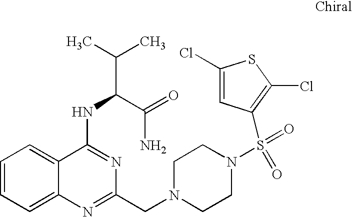 | 23-51 | 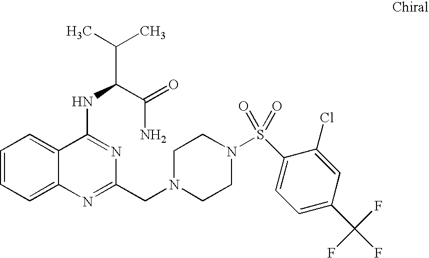 |
| 23-45 | 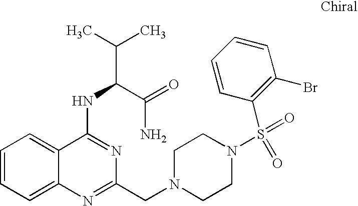 | 23-52 | 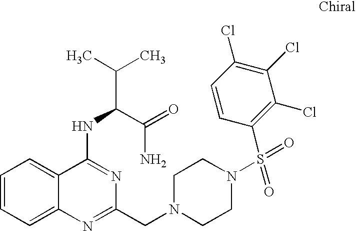 |
| 23-46 | 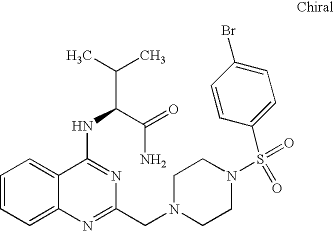 | 23-53 | 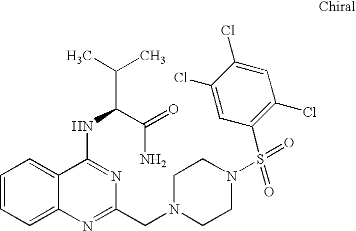 |
| 23-47 | 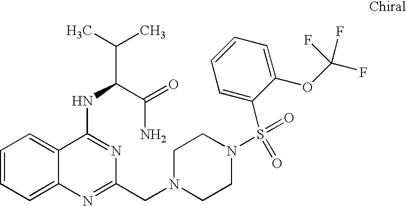 | 23-54 | 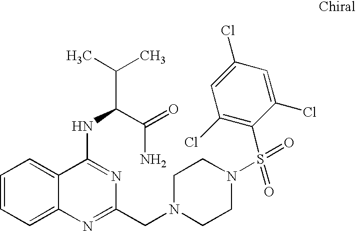 |
| 23-48 | 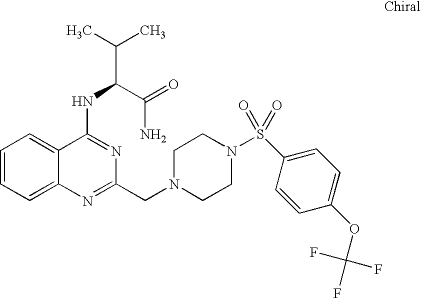 | 23-55 | 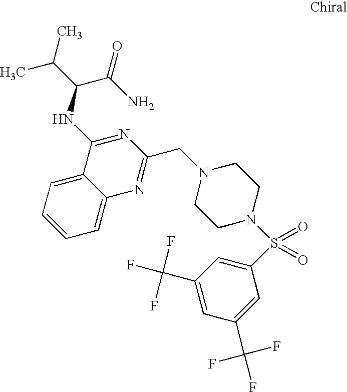 |
| 23-56 | 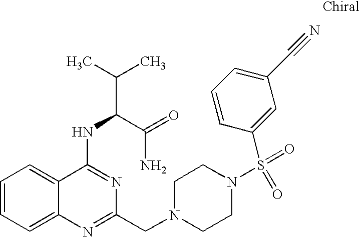 | 23-63 | 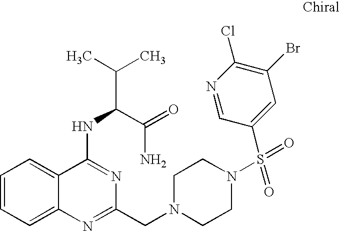 |
| 23-57 | 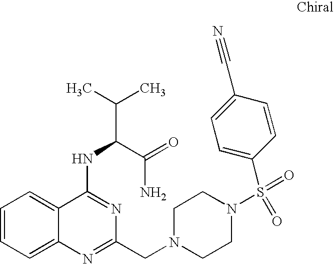 | 23-64 | 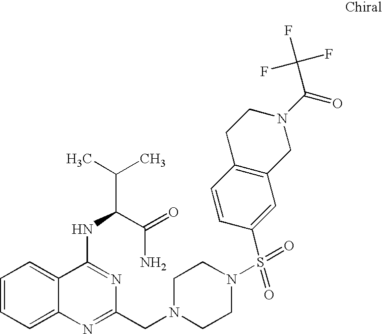 |
| 23-58 | 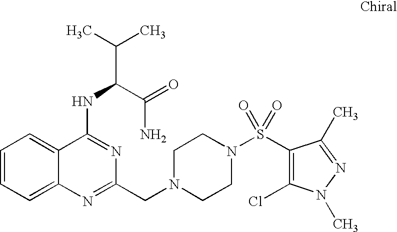 | 23-65 | 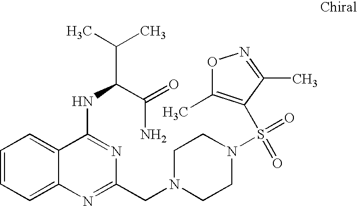 |
| 23-59 | 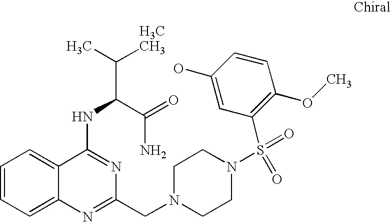 | 23-66 | 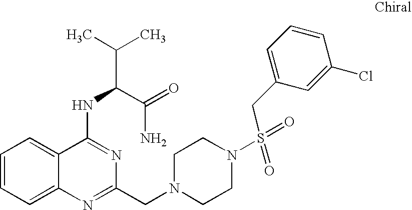 |
| 23-60 | 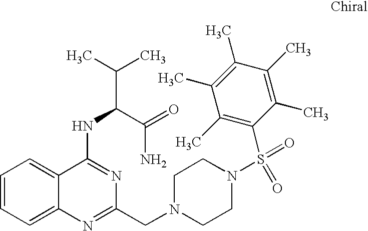 | 23-67 | 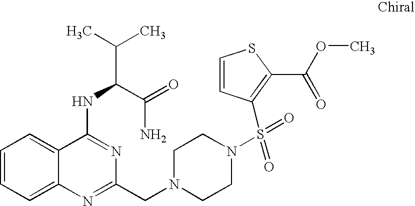 |
| 23-61 | 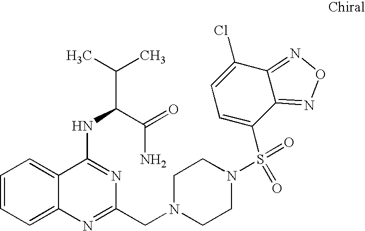 | 23-68 | 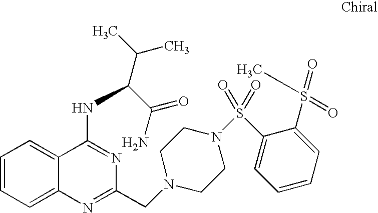 |
| 23-62 | 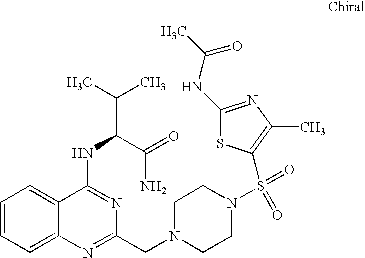 | 23-69 | 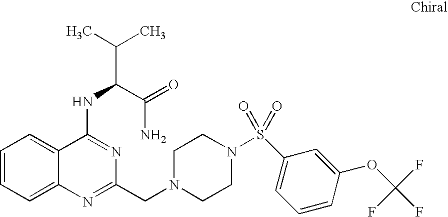 |
| 23-70 | 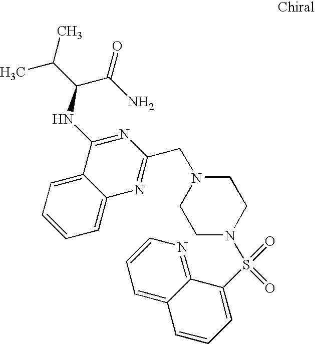 | 23-77 | 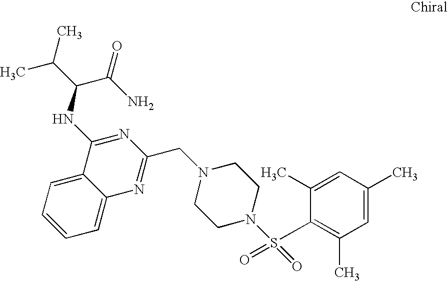 |
| 23-71 | 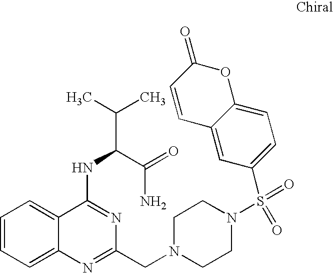 | 24-1 | 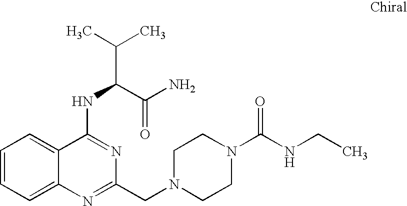 |

| | | | |
|---|---|---|---|
| 23-72 | 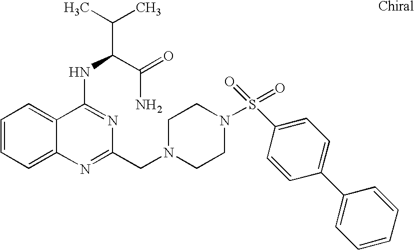 | 24-2 | 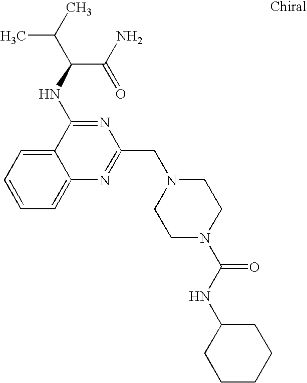 |
| 23-73 | 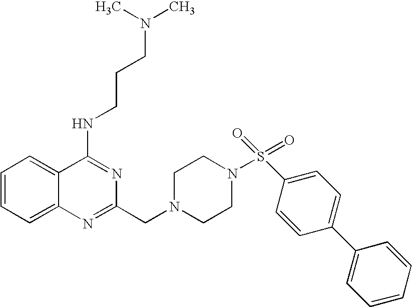 | 24-3 | 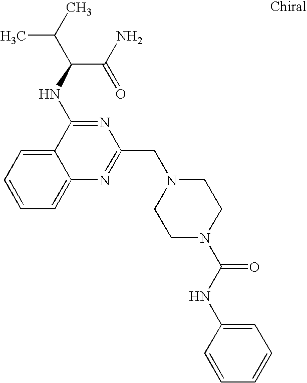 |
| 23-74 | 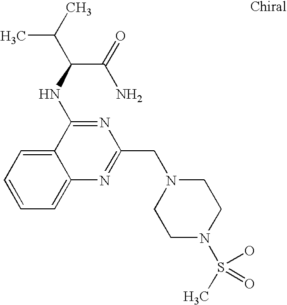 | 24-4 | 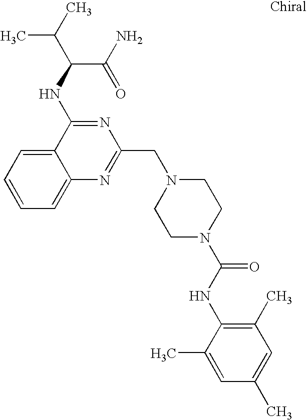 |
| 23-75 | 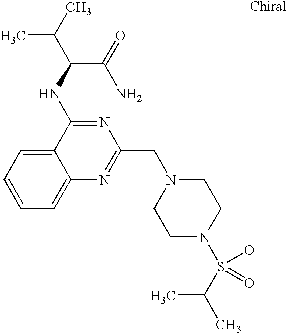 | 24-5 | 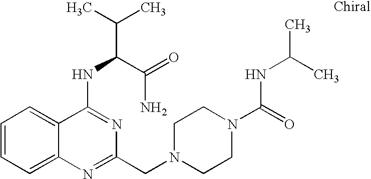 |
| 23-76 | 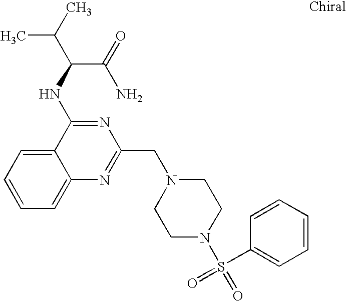 | 24-6 | 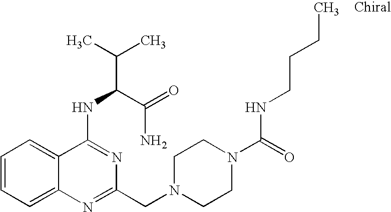 |
| 24-7 | 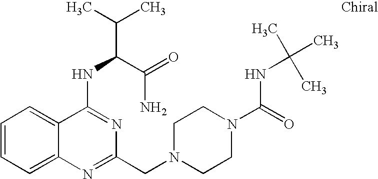 | 24-14 | 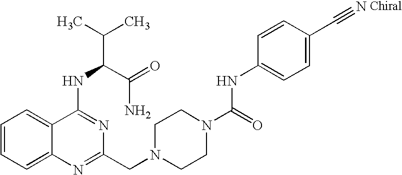 |
| 24-8 | 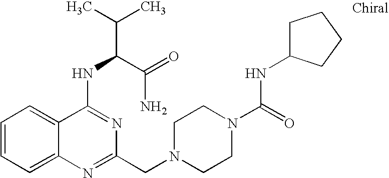 | 24-15 | 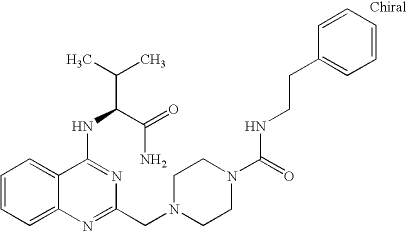 |
| 24-9 | 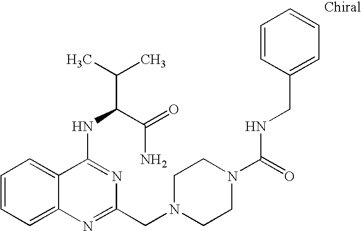 | 24-16 | 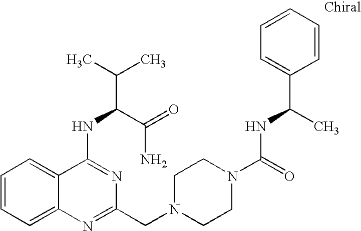 |
| 24-10 | 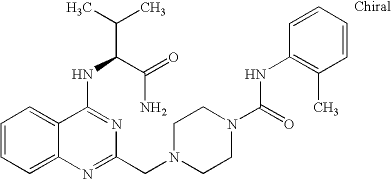 | 24-17 | 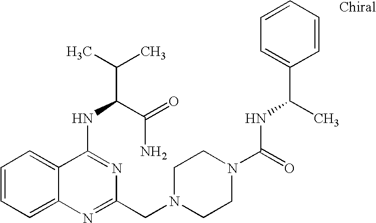 |
| 24-11 | 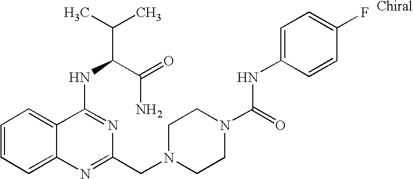 | 24-18 | 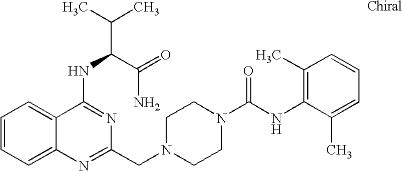 |
| 24-12 | 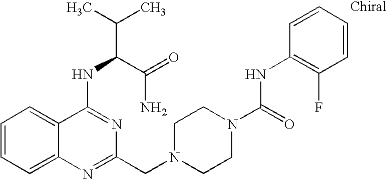 | 24-19 | 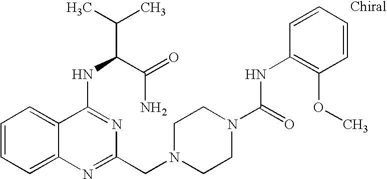 |
| 24-13 | 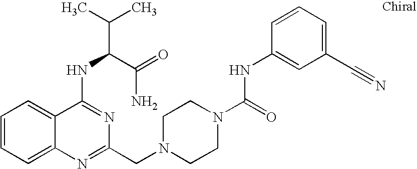 | 24-20 | 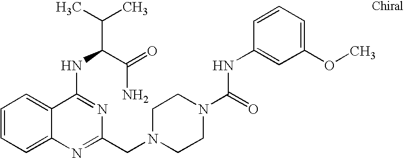 |
| 24-21 | 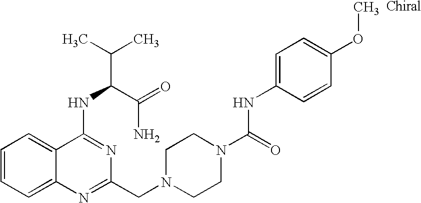 | 24-28 | 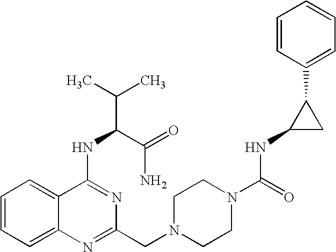 |
| 24-22 | 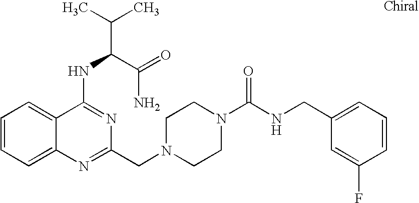 | 24-29 | 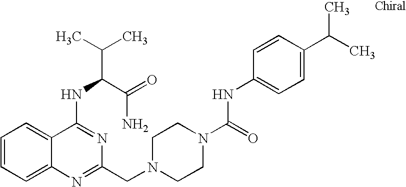 |
| 24-23 | 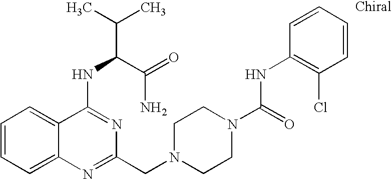 | 24-30 | 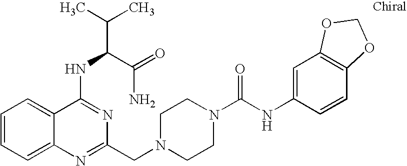 |
| 24-24 | 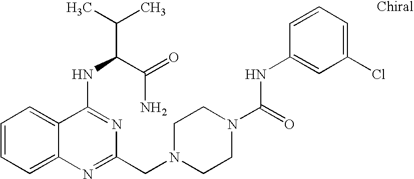 | 24-31 | 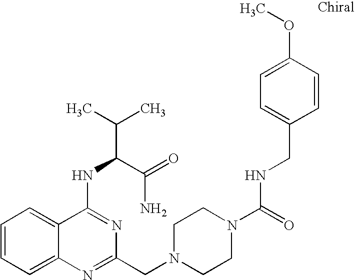 |
| 24-25 | 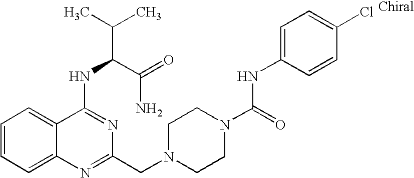 | 24-32 | 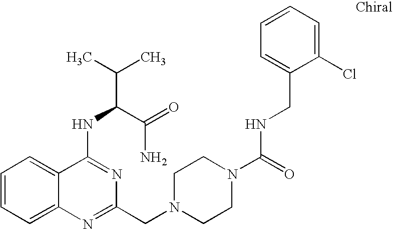 |
| 24-26 | 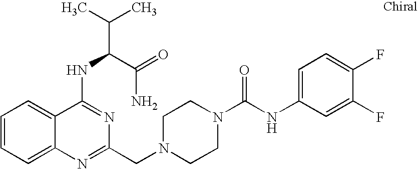 | 24-33 | 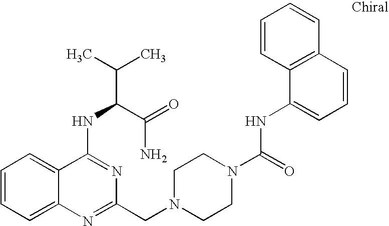 |
| 24-27 | 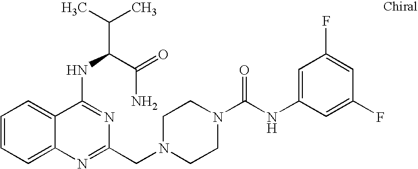 | 24-34 | 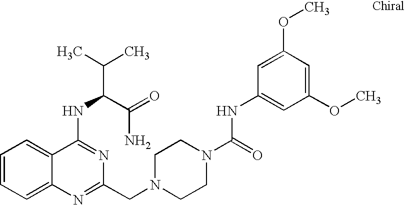 |
| 24-35 | 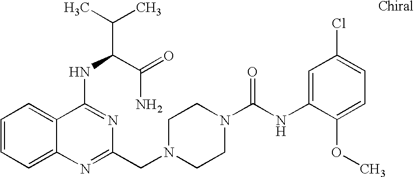 | 24-42 | 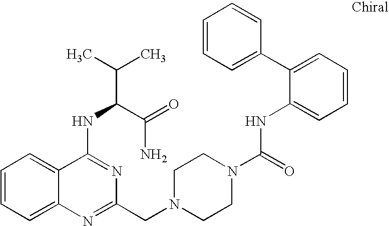 |
| 24-36 | 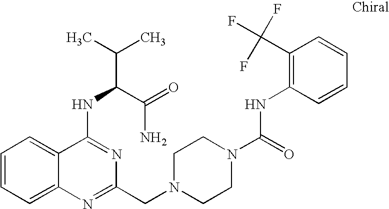 | 24-43 | 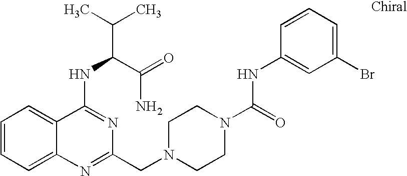 |
| 24-37 | 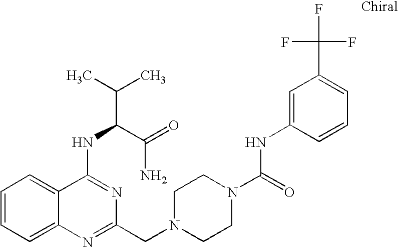 | 24-44 | 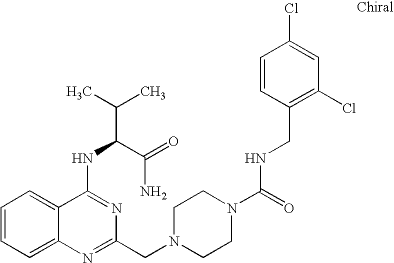 |
| 24-38 | 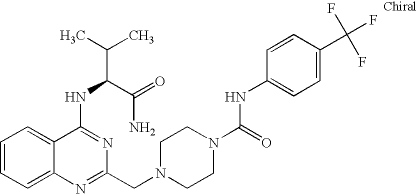 | 24-45 | 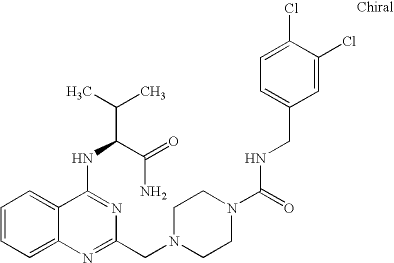 |
| 24-39 | 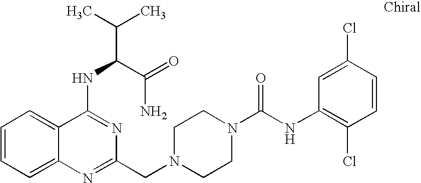 | 24-46 | 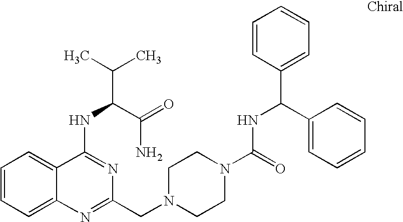 |
| 24-40 | 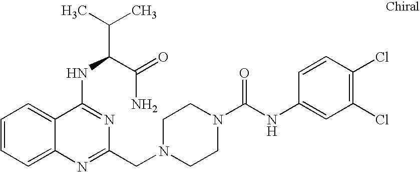 | 24-47 | 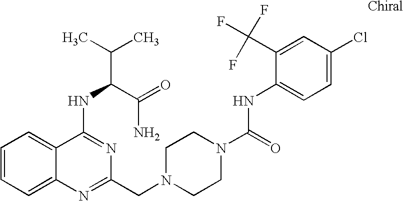 |
| 24-41 | 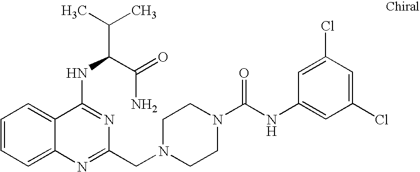 | 24-48 | 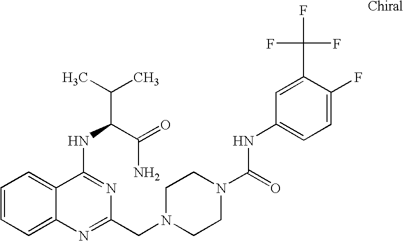 |
| 24-49 | 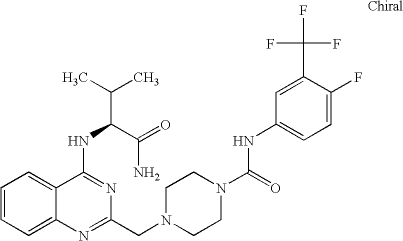 | 25-7 | 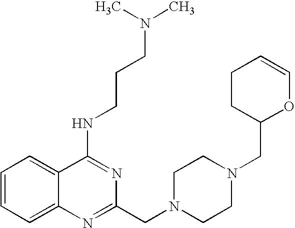 |
| 25-1 | 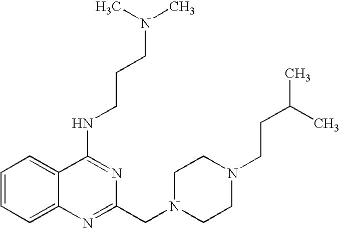 | 24-8 | 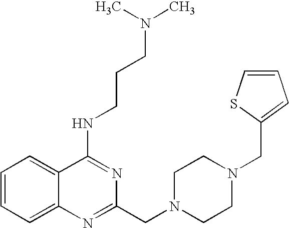 |
| 25-2 | 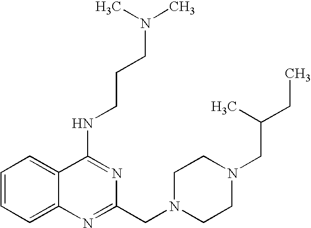 | 25-9 | 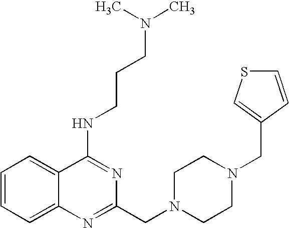 |
| 25-3 | 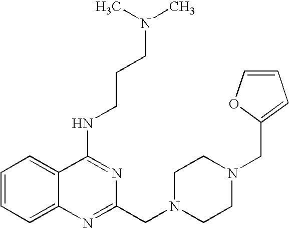 | 25-10 | 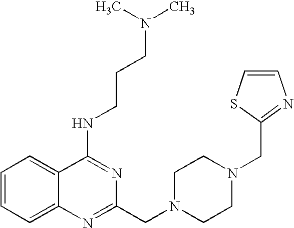 |
| 25-4 | 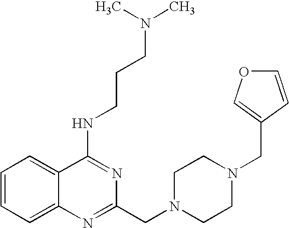 | 25-11 | 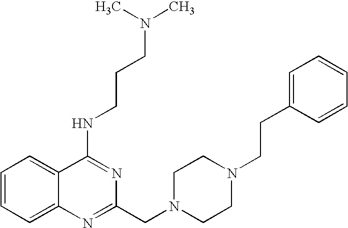 |
| 25-5 | 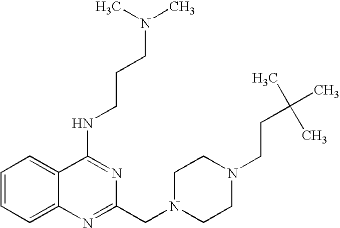 | 25-12 | 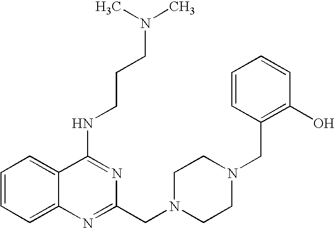 |
| 25-6 | 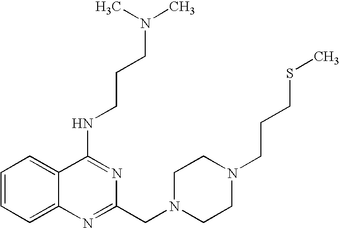 | 25-13 | 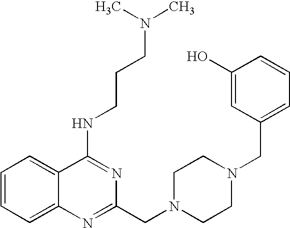 |
| 25-14 | 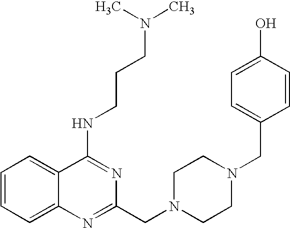 | 25-21 | 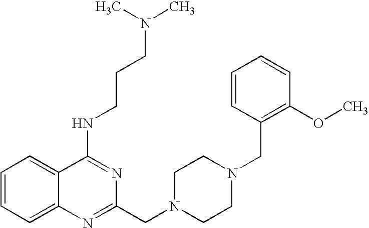 |
| 25-15 | 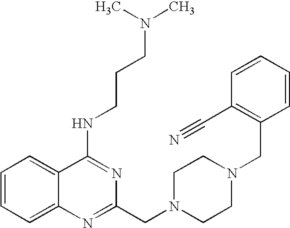 | 25-22 | 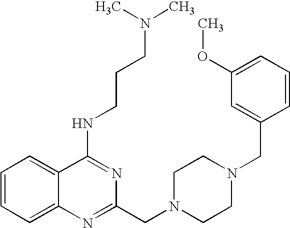 |
| 25-16 | 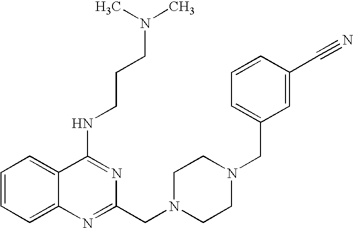 | 25-23 | 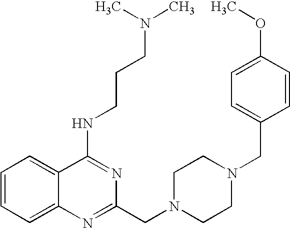 |
| 25-17 | 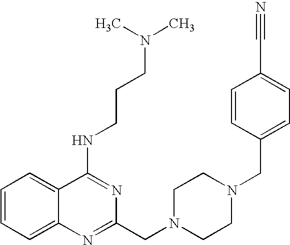 | 25-24 | 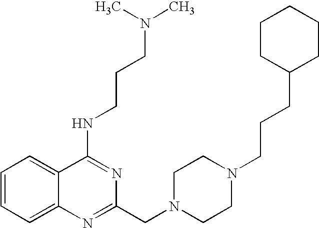 |
| 25-18 | 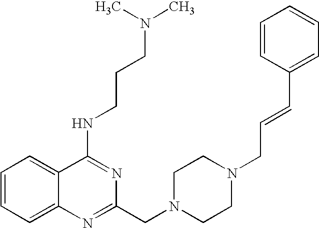 | 25-25 | 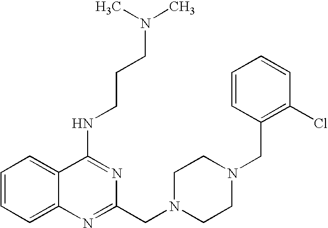 |
| 25-19 | 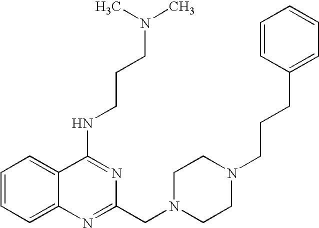 | 25-26 | 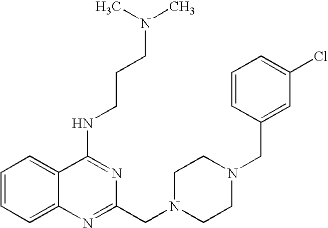 |
| 25-20 | 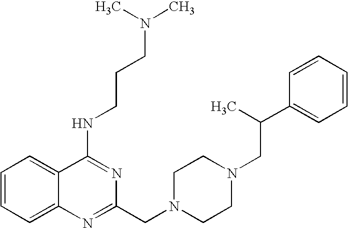 | 25-27 | 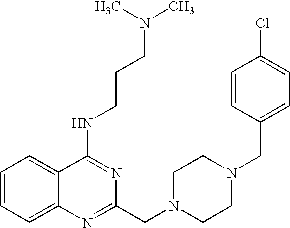 |
| 25-28 | 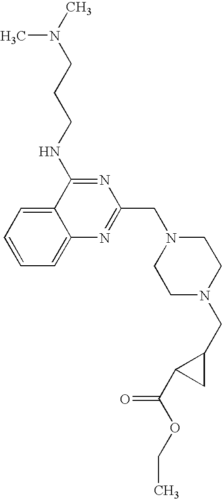 | 25-35 | 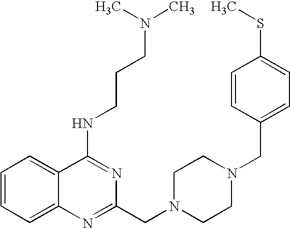 |
| 25-29 | 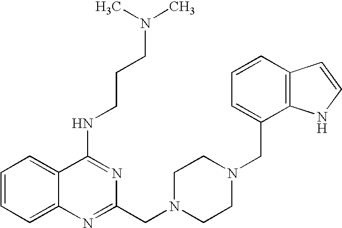 | 25-36 | 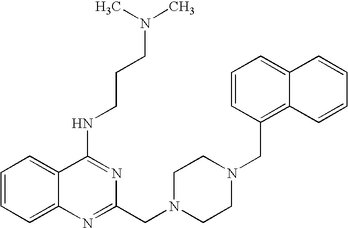 |
| 25-30 | 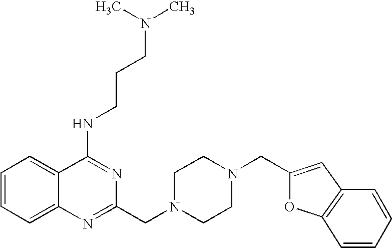 | 25-37 | 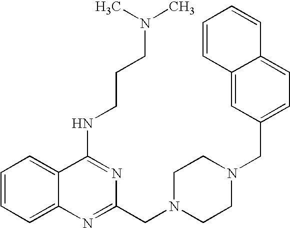 |
| 25-31 | 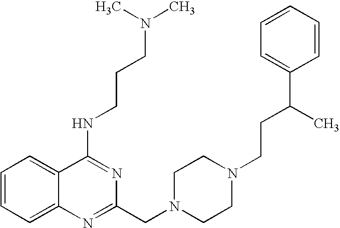 | 25-38 | 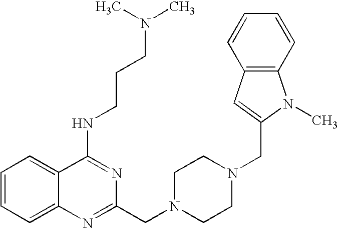 |
| 25-32 | 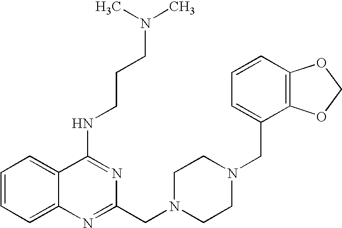 | 25-39 | 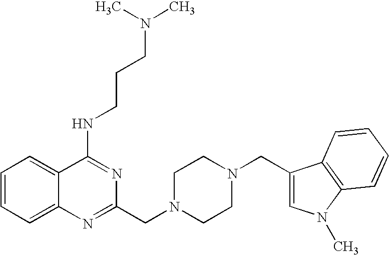 |
| 25-33 | 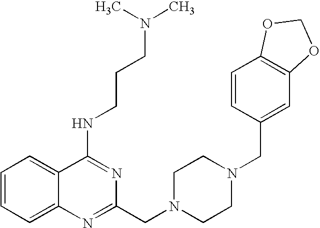 | 25-40 | 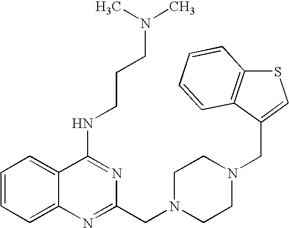 |
| 25-34 | 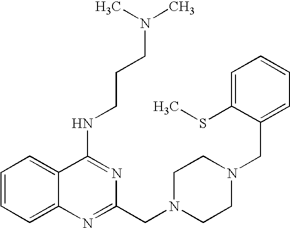 | 25-41 | 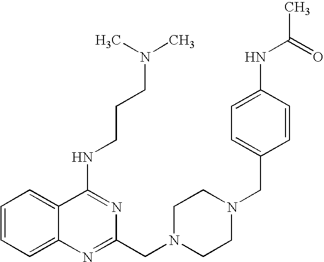 |
| 25-42 | 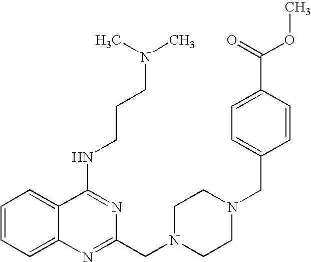 | 25-49 | 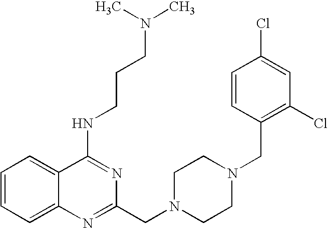 |
| 25-43 | 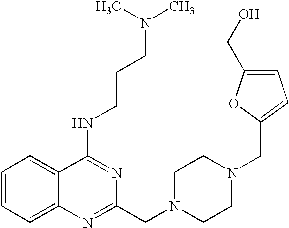 | 25-50 | 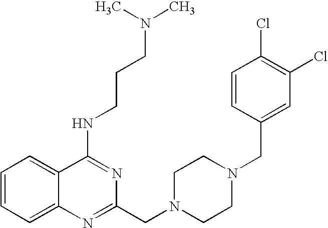 |
| 25-44 | 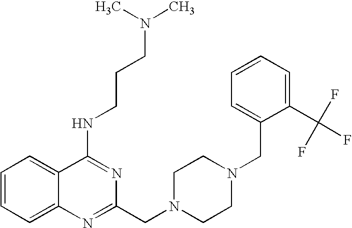 | 25-51 | 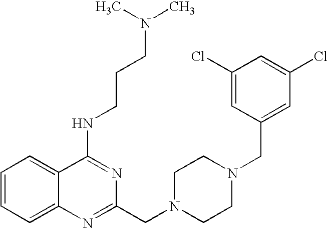 |
| 25-45 | 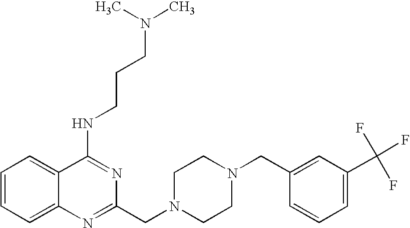 | 25-52 | 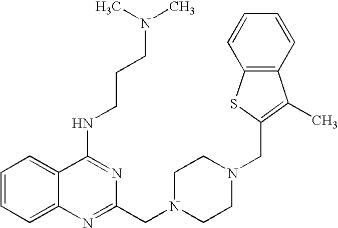 |
| 25-46 | 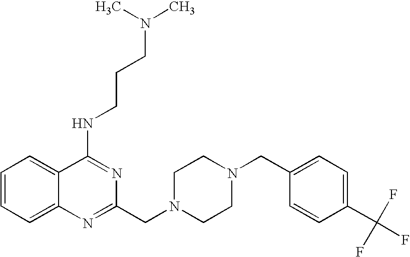 | 25-53 | 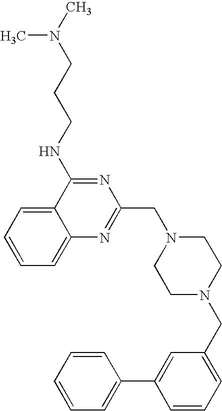 |
| 25-47 | 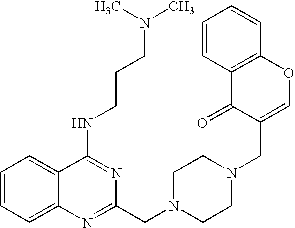 | 25-54 | 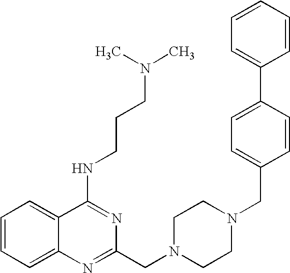 |
| 25-48 | 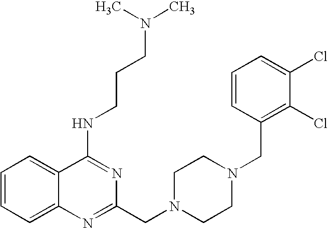 | 25-55 | 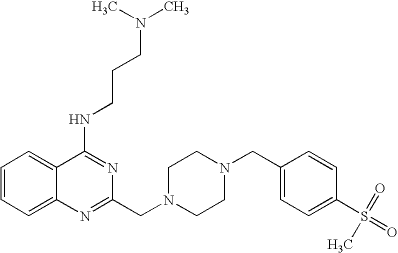 |
| 25-56 | 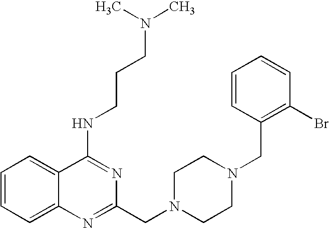 | 25-63 | 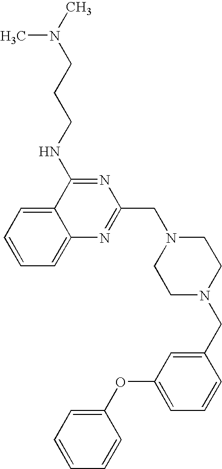 |
| 25-57 | 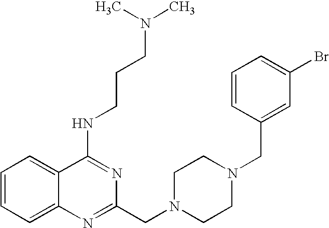 | 25-64 | 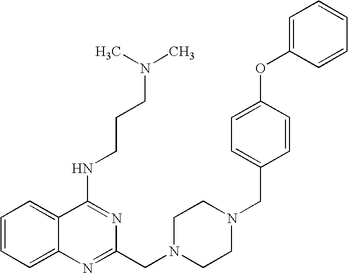 |
| 25-58 | 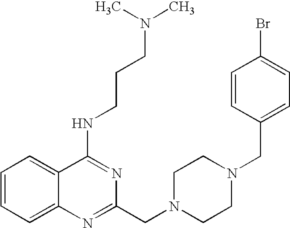 | 25-65 | 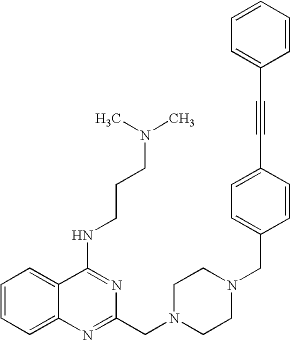 |
| 25-59 | 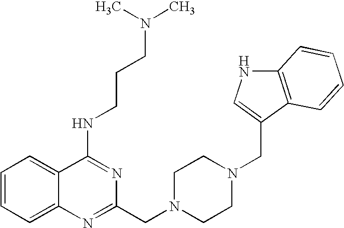 | 25-66 | 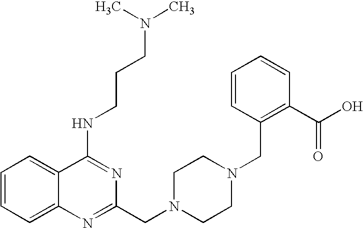 |
| 25-60 | 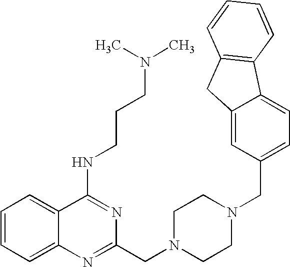 | 25-67 | 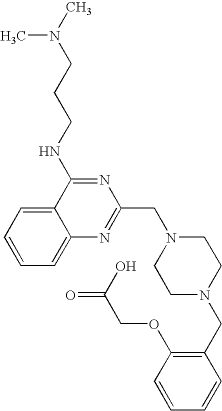 |
| 25-61 | 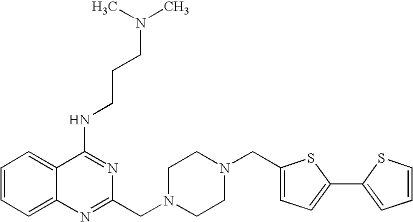 | 25-68 | 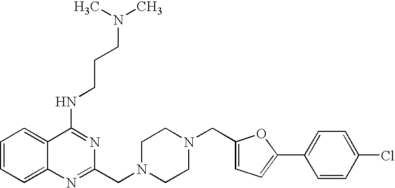 |
| 25-62 | 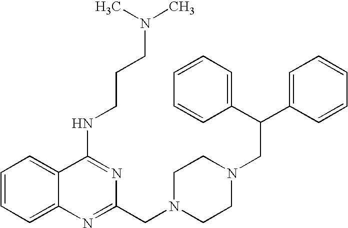 | 25-69 | 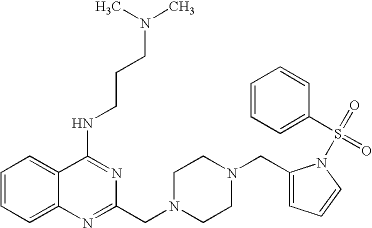 |
| 25-70 | 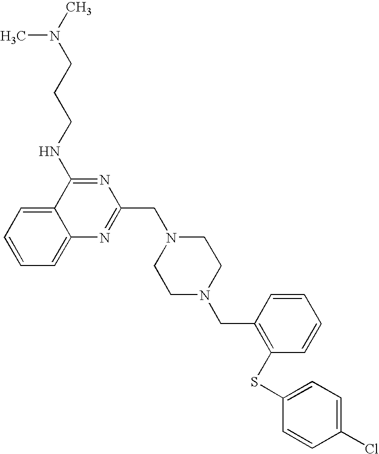 | 25-77 | 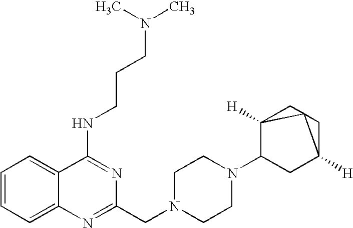 |
| 25-71 | 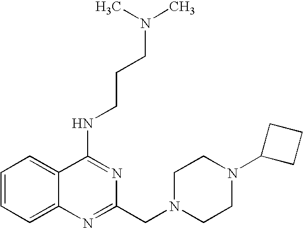 | 25-78 | 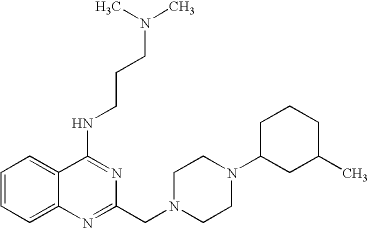 |
| 25-72 | 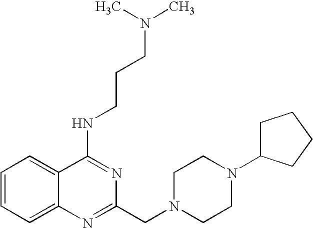 | 25-79 | 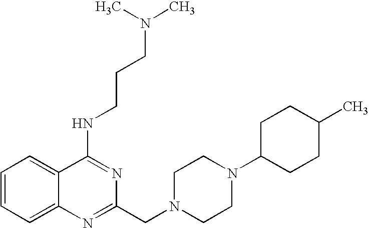 |
| 25-73 | 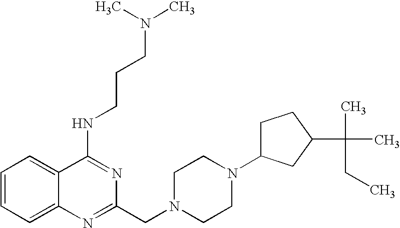 | 25-80 | 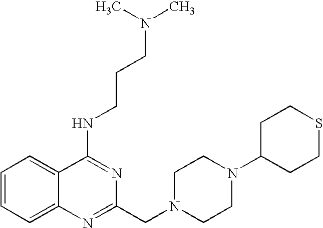 |
| 25-74 | 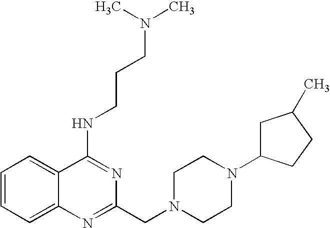 | 25-81 | 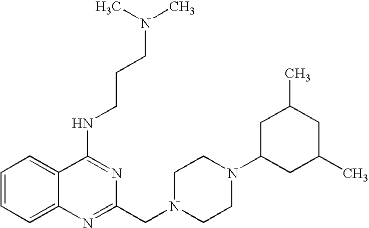 |
| 25-75 | 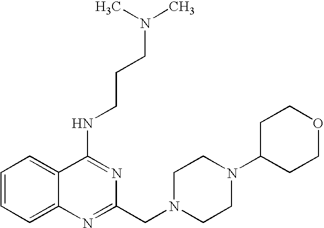 | 25-82 | 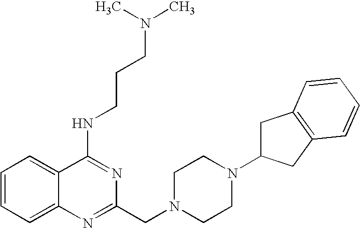 |
| 25-76 | 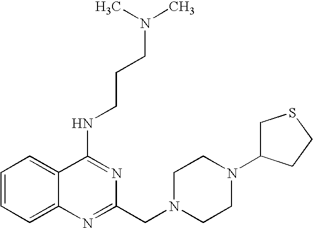 | 25-83 | 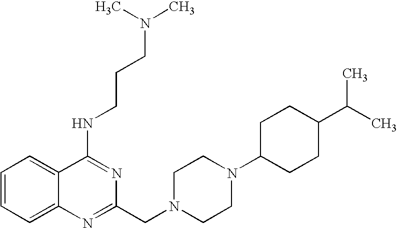 |
| 25-84 | 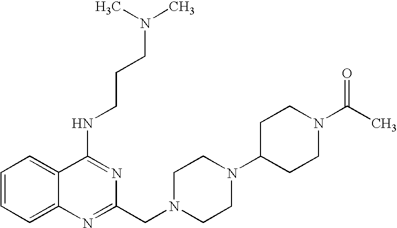 | 25-91 | 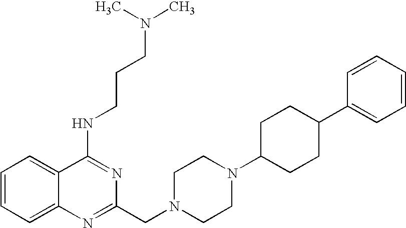 |
| 25-85 | 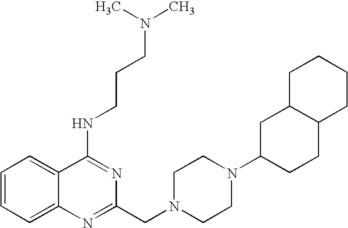 | 25-92 | 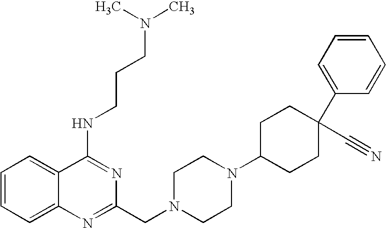 |
| 25-86 | 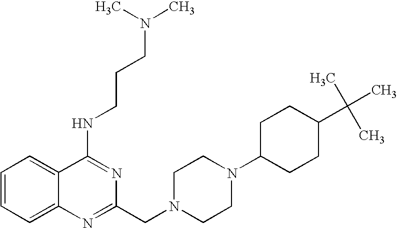 | 25-93 | 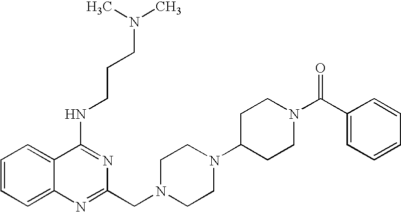 |
| 25-87 | 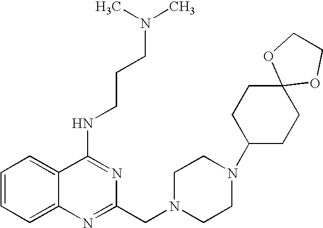 | 25-94 | 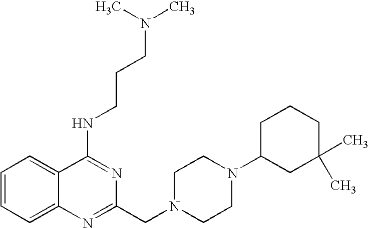 |
| 25-88 | 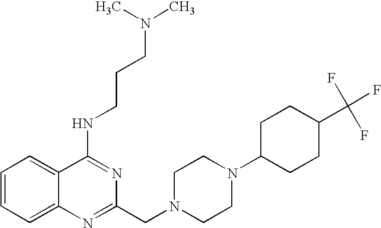 | 26-1 | 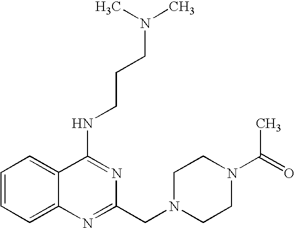 |
| 25-89 | 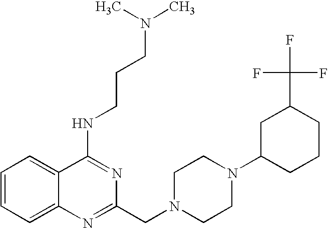 | 26-2 | 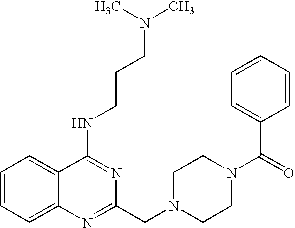 |
| 25-90 | 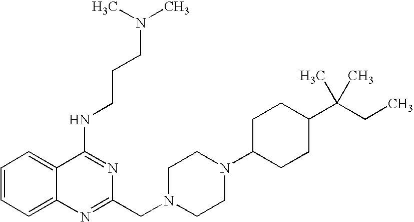 | 26-3 | 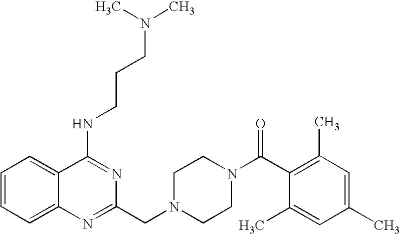 |
| 26-4 | 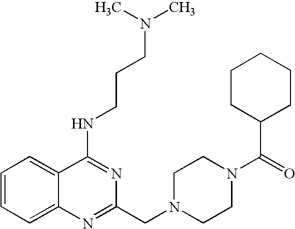 | 26-11 | 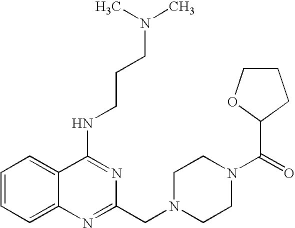 |
| 26-5 | 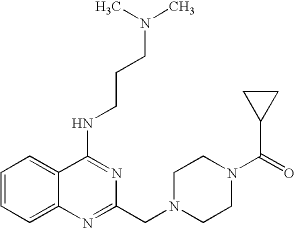 | 26-12 | 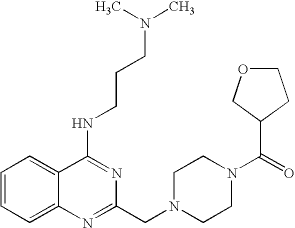 |
| 26-6 | 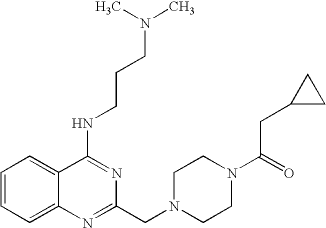 | 26-13 | 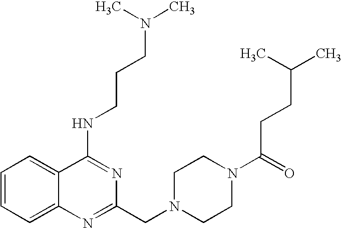 |
| 26-7 | 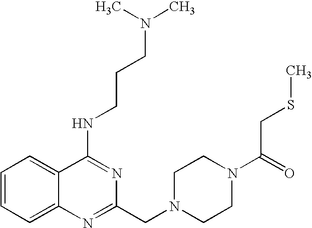 | 26-14 | 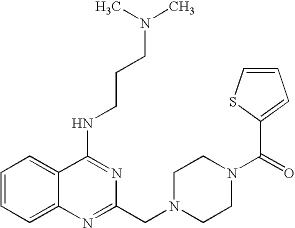 |
| 26-8 | 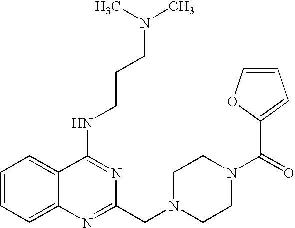 | 26-15 | 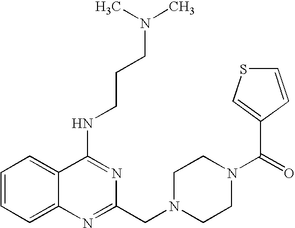 |
| 26-9 | 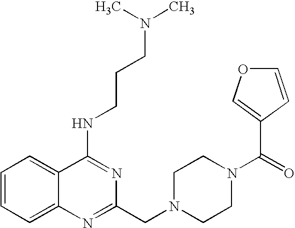 | 26-16 | 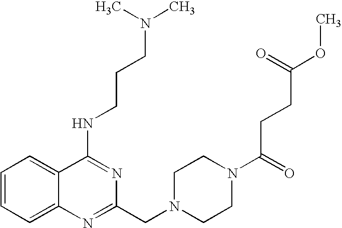 |
| 26-10 | 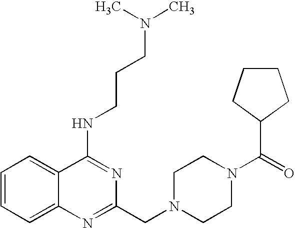 | 26-17 | 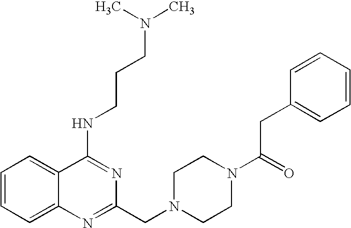 |
| 26-18 | 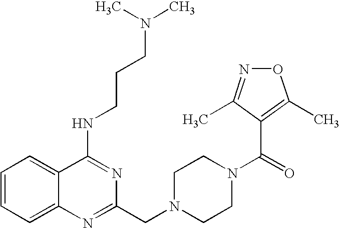 | 26-25 | 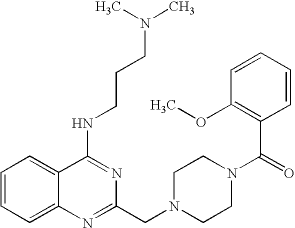 |
| 26-19 | 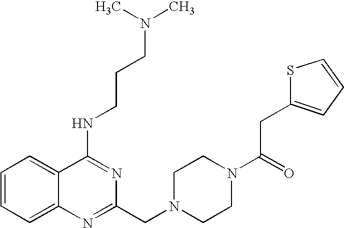 | 26-26 | 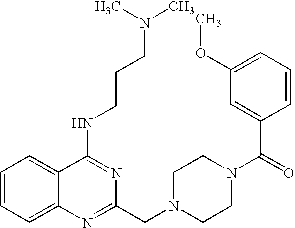 |
| 26-20 | 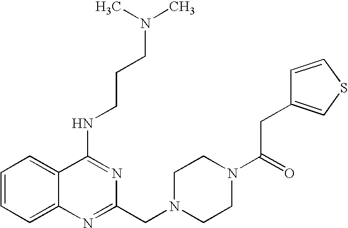 | 26-27 | 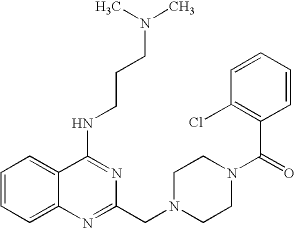 |
| 26-21 | 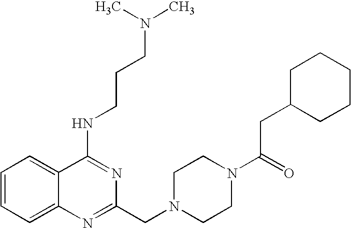 | 26-28 | 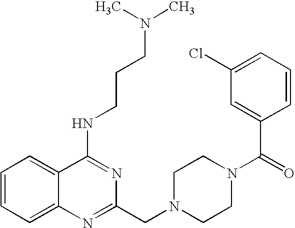 |
| 26-22 | 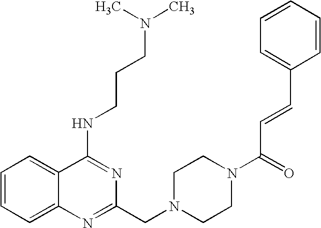 | 26-29 | 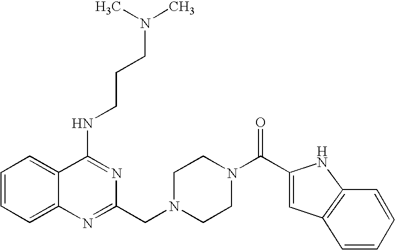 |
| 26-23 | 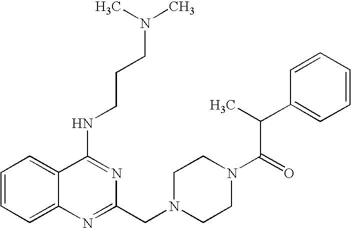 | 26-30 | 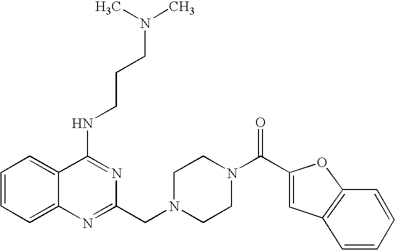 |
| 26-24 | 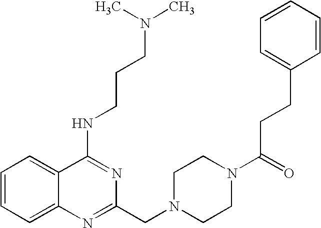 | 26-31 | 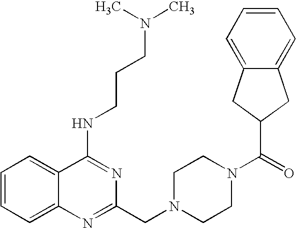 |
| 26-32 | 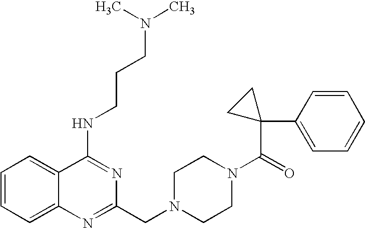 | 26-39 | 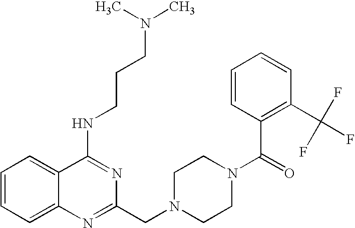 |
| 26-33 | 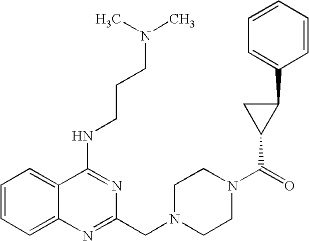 | 26-40 | 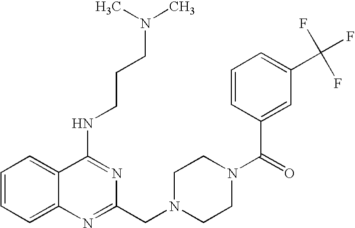 |
| 26-34 | 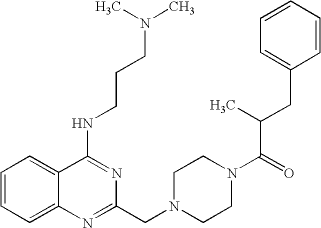 | 26-41 | 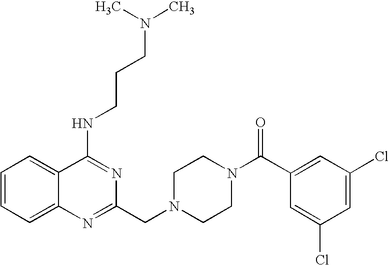 |
| 26-35 | 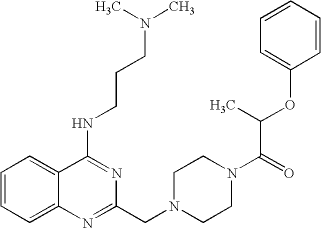 | 26-42 | 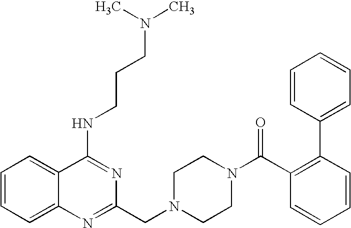 |
| 26-36 | 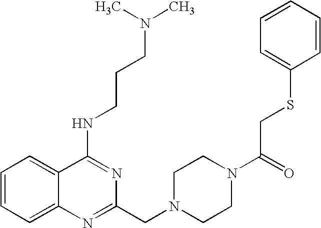 | 26-43 | 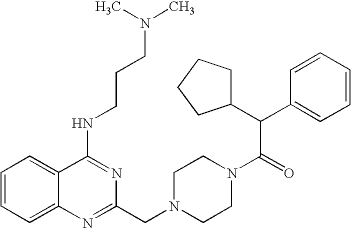 |
| 26-37 | 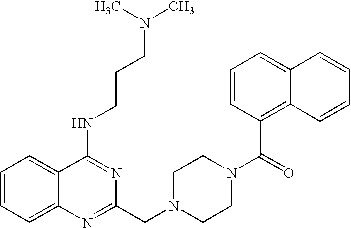 | 26-44 | 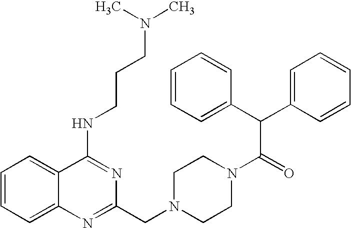 |
| 26-38 | 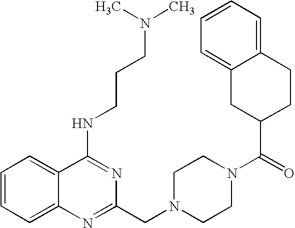 | 26-45 | 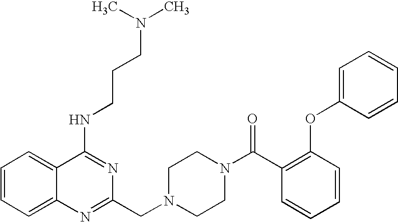 |
| 26-46 | 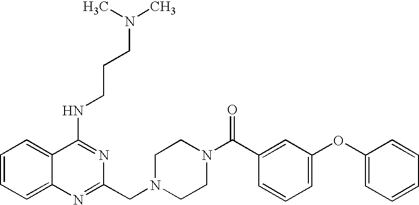 | 26-53 | 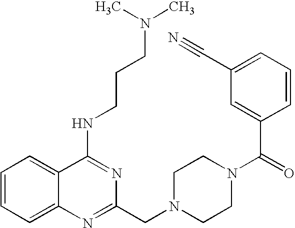 |
| 26-47 | 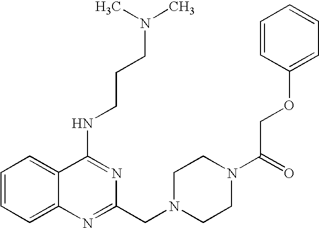 | 26-54 | 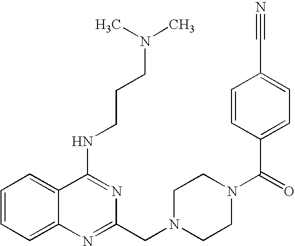 |
| 26-48 | 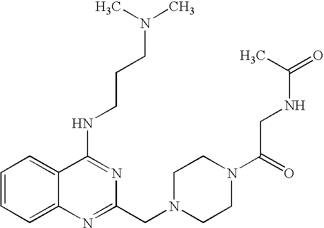 | 26-55 | 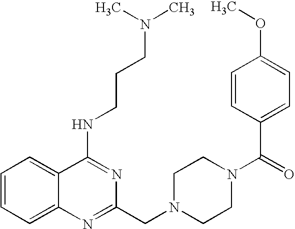 |
| 26-49 | 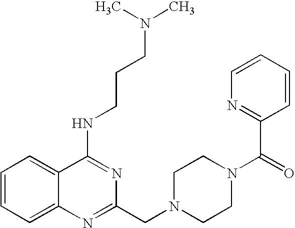 | 26-56 | 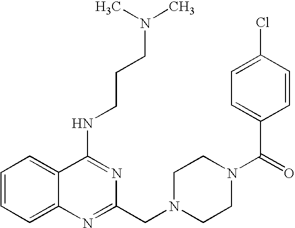 |
| 26-50 | 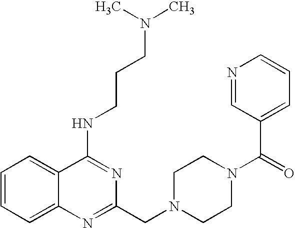 | 26-57 | 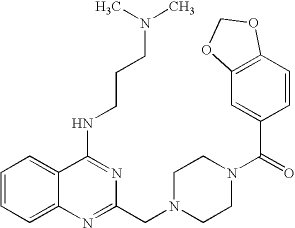 |
| 26-51 | 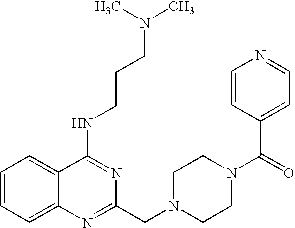 | 26-58 | 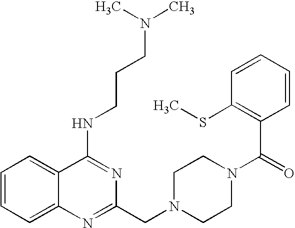 |
| 26-52 | 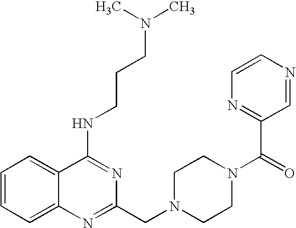 | 26-59 | 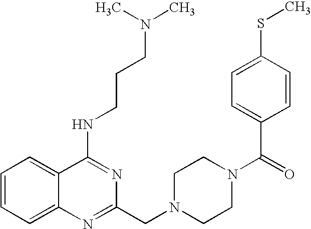 |
| 26-60 | 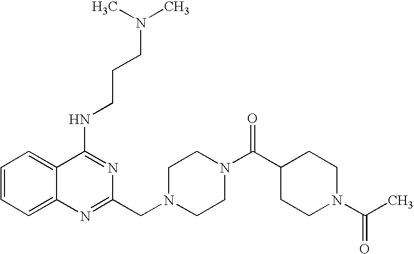 | 26-67 | 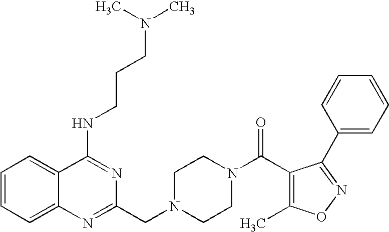 |
| 26-61 | 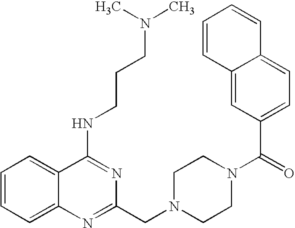 | 26-68 | 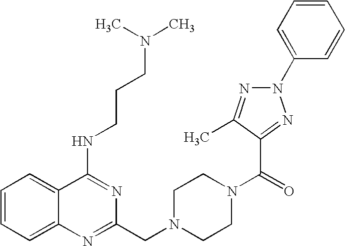 |
| 26-62 | 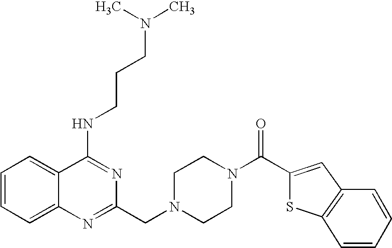 | 26-69 | 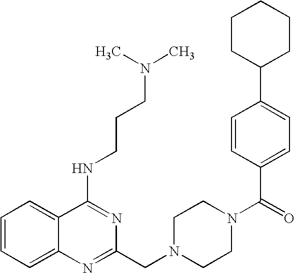 |
| 26-63 | 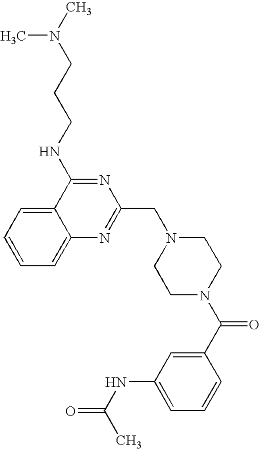 | 26-70 | 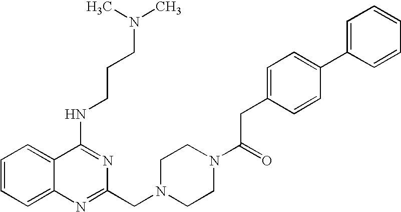 |
| 26-64 | 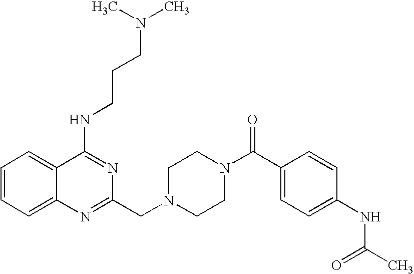 | 27-1 | 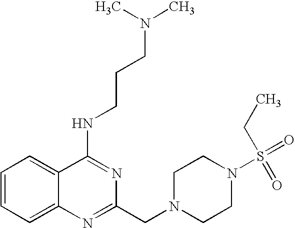 |
| 26-65 | 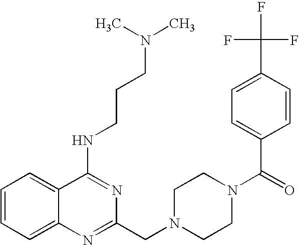 | 27-2 | 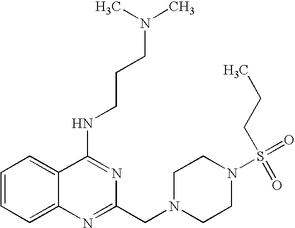 |
| 26-66 | 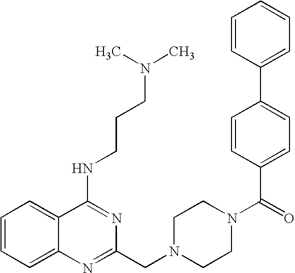 | 27-3 | 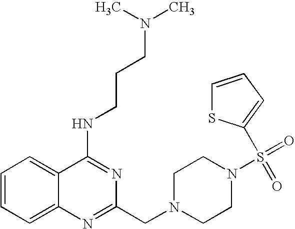 |
| 27-4 | 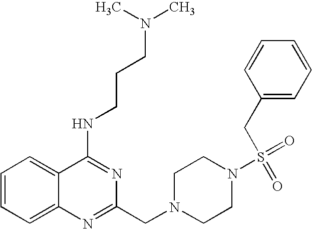 | 27-11 | 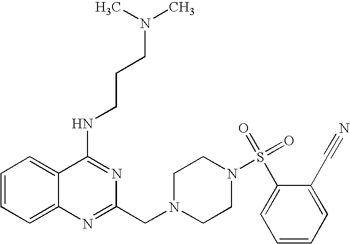 |
| 27-5 | 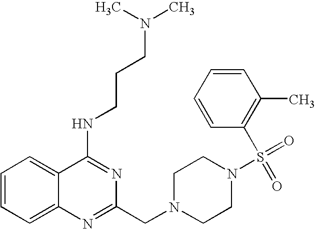 | 27-12 | 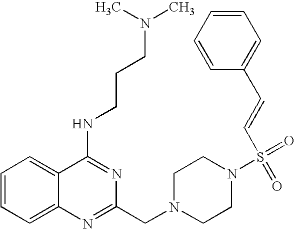 |
| 27-6 | 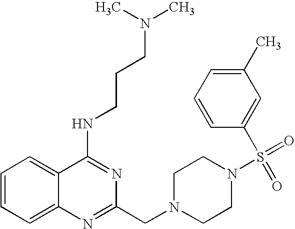 | 27-13 | 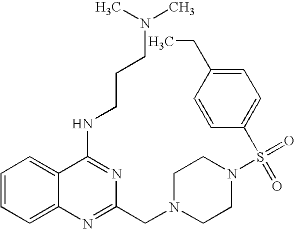 |
| 27-7 | 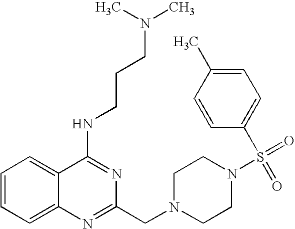 | 27-14 | 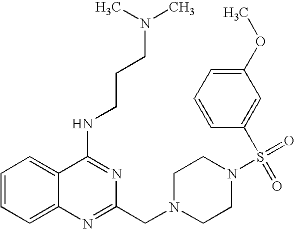 |
| 27-8 | 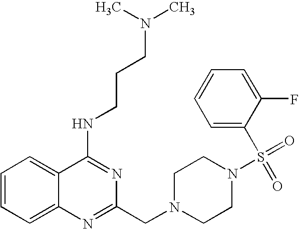 | 27-15 | 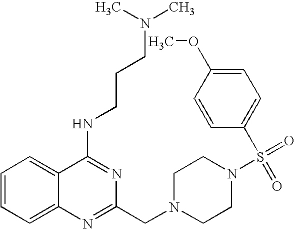 |
| 27-9 | 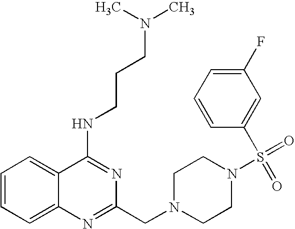 | 27-16 | 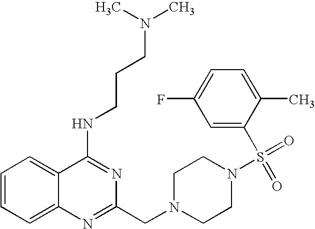 |
| 27-10 | 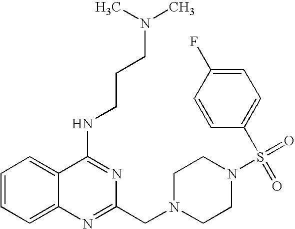 | 27-17 | 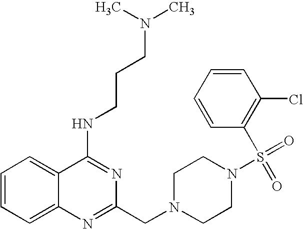 |
| 27-18 | 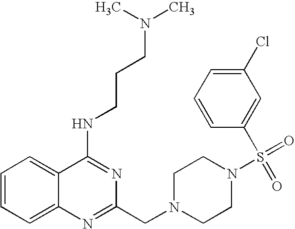 | 27-25 | 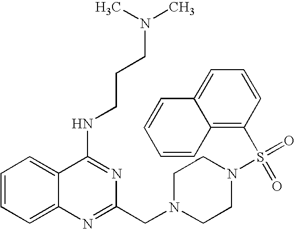 |
| 27-19 | 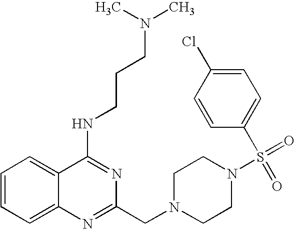 | 27-26 | 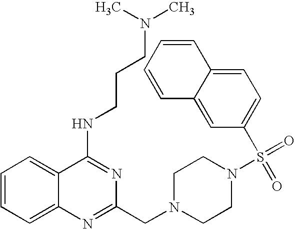 |
| 27-20 | 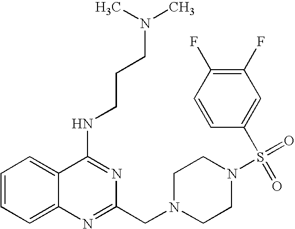 | 27-27 | 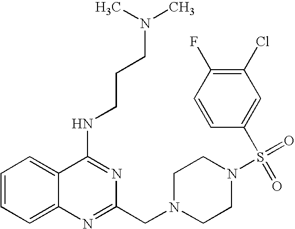 |
| 27-21 | 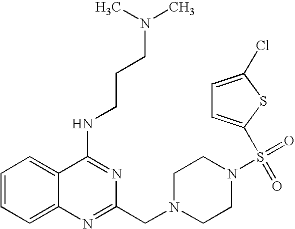 | 27-28 | 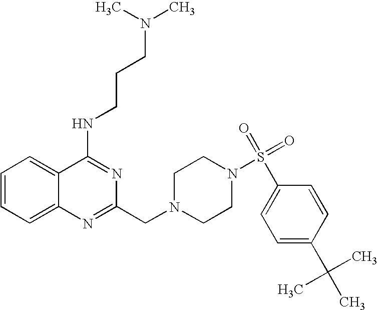 |
| 27-22 | 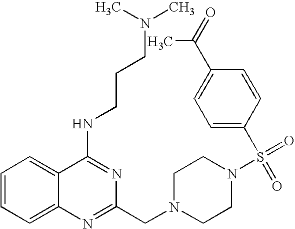 | 27-29 | 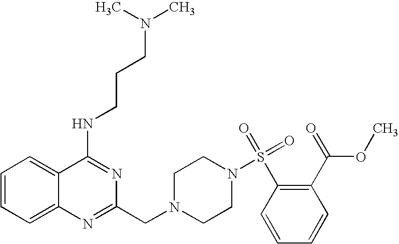 |
| 27-23 | 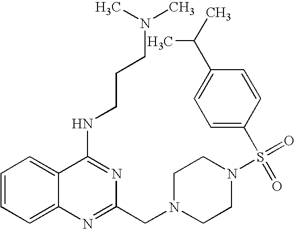 | 27-30 | 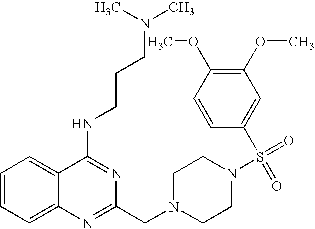 |
| 27-24 | 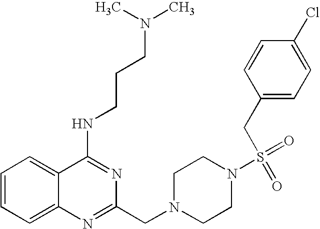 | 27-31 | 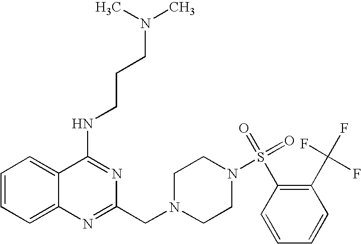 |
| 27-32 | 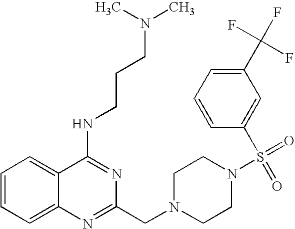 | 27-39 | 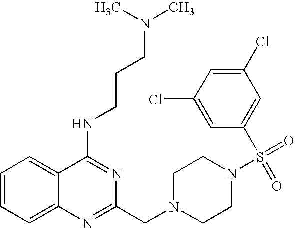 |
| 27-33 | 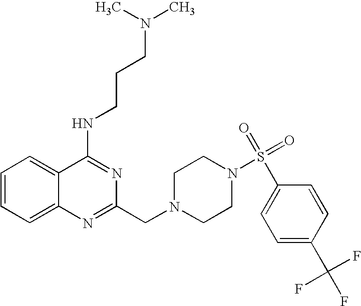 | 27-40 | 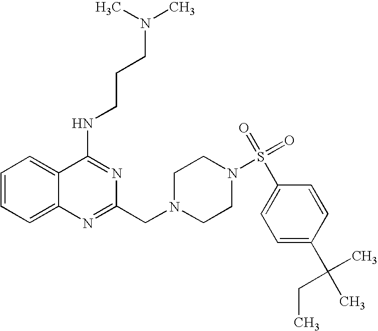 |
| 27-34 | 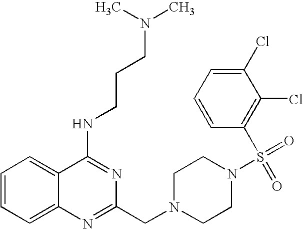 | 27-41 | 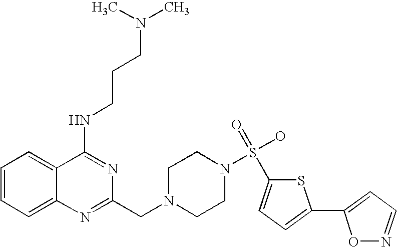 |
| 27-35 | 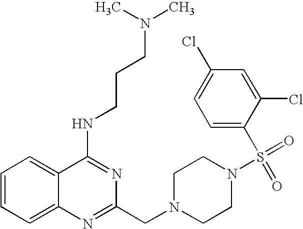 | 27-42 | 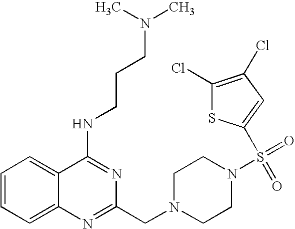 |
| 27-36 | 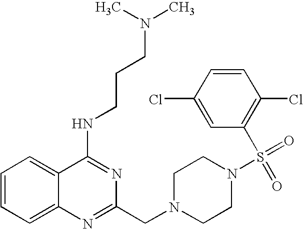 | 27-43 | 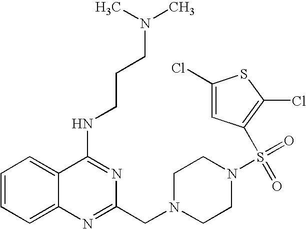 |
| 27-37 | 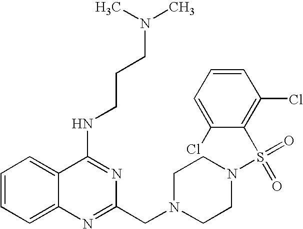 | 27-44 | 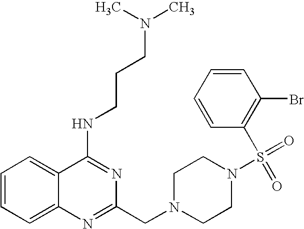 |
| 27-38 | 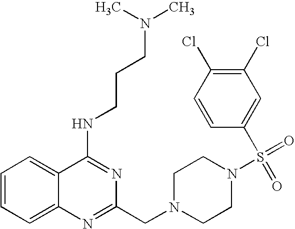 | 27-45 | 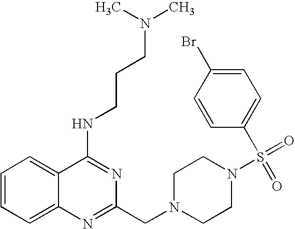 |
| 27-46 | 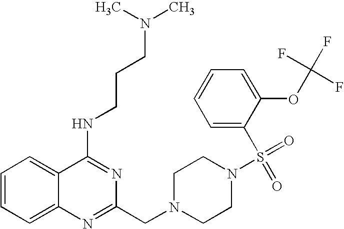 | 27-53 | 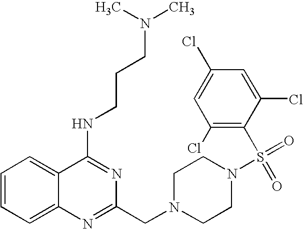 |
| 27-47 | 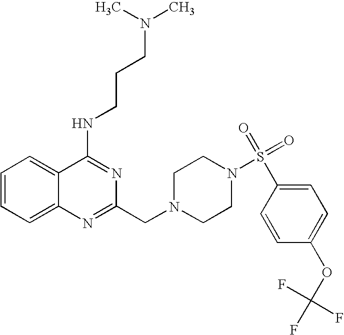 | 27-54 | 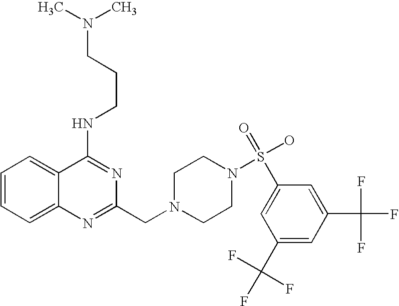 |
| 27-48 | 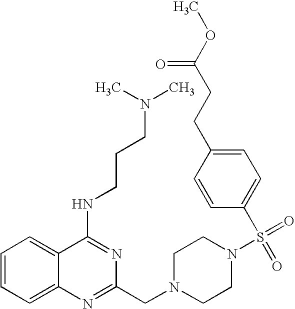 | 27-55 | 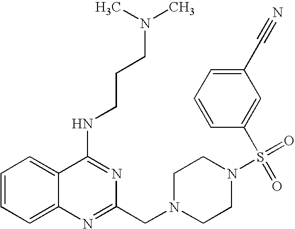 |
| 27-49 | 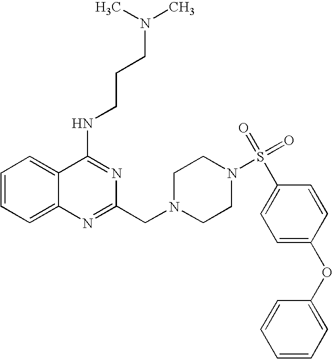 | 27-56 | 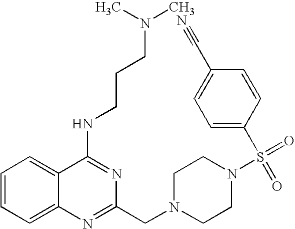 |
| 27-50 | 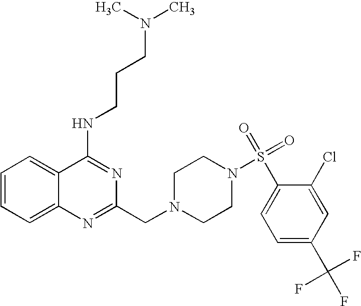 | 27-57 | 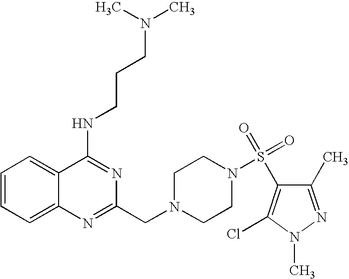 |
| 27-51 | 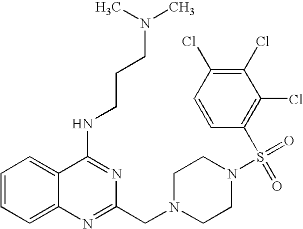 | 27-58 | 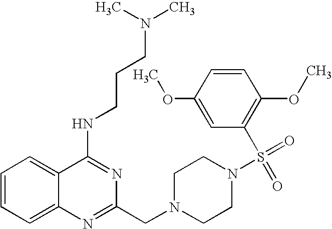 |
| 27-52 | 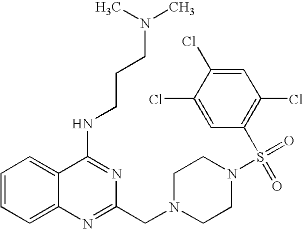 | 27-59 | 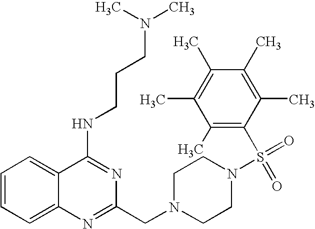 |
| 27-60 | 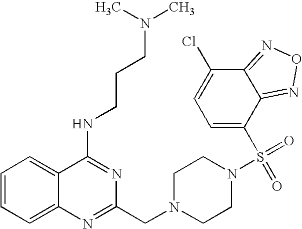 | 27-67 | 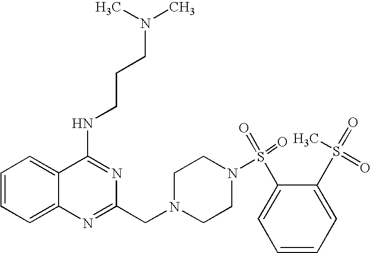 |
| 27-61 | 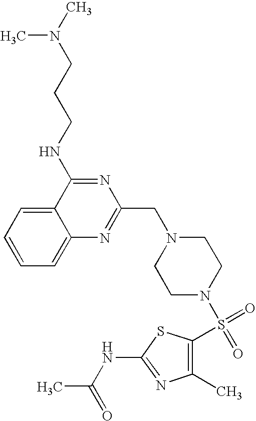 | 27-68 | 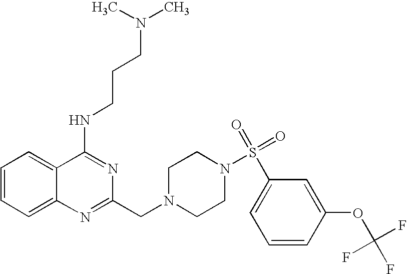 |
| 27-62 | 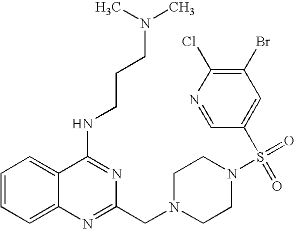 | 27-69 | 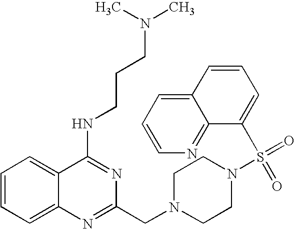 |
| 27-63 | 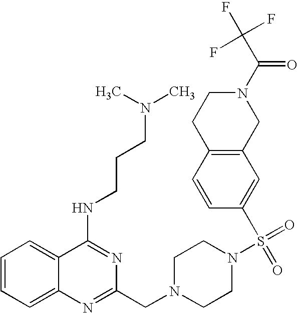 | 27-70 | 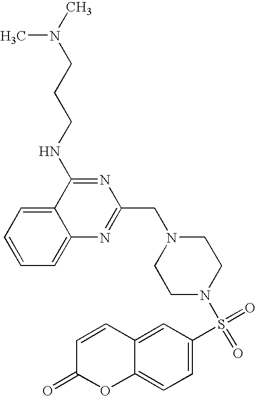 |
| 27-64 | 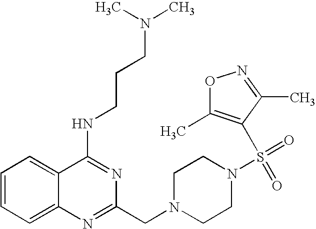 | 27-71 | 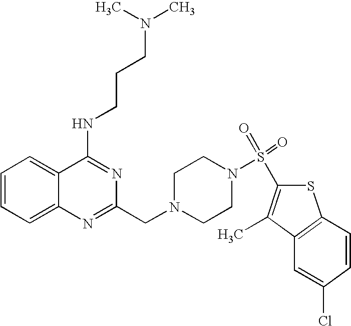 |
| 27-65 | 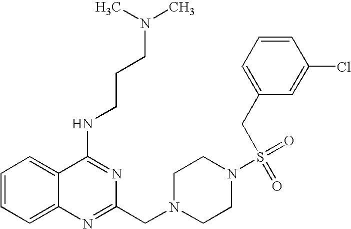 | 27-72 | 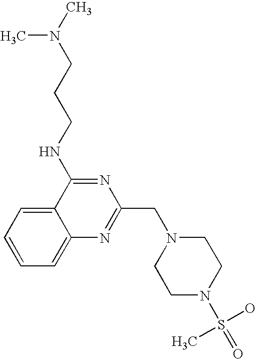 |
| 27-66 | 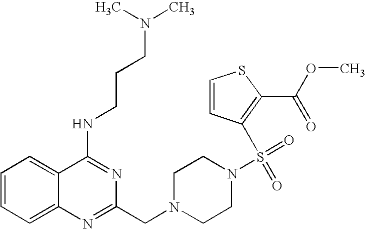 | 27-73 | 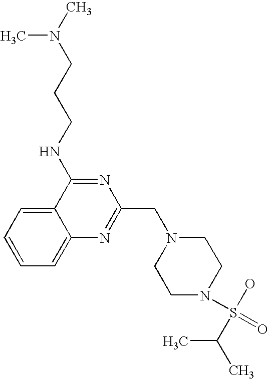 |
| 27-74 | 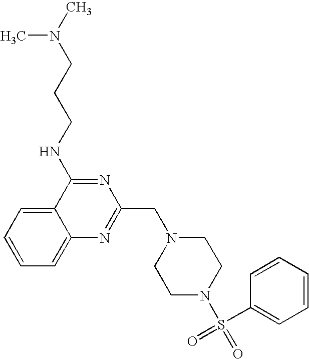 | 28-6 | 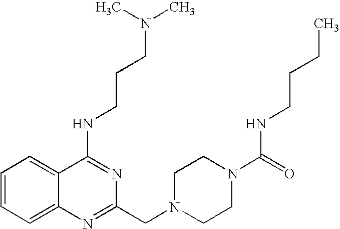 |
| 27-75 | 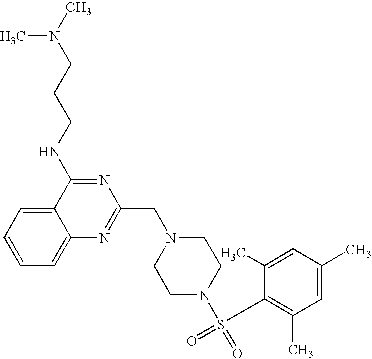 | 28-7 | 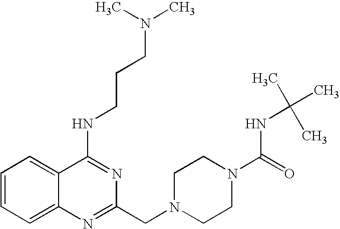 |
| 28-1 | 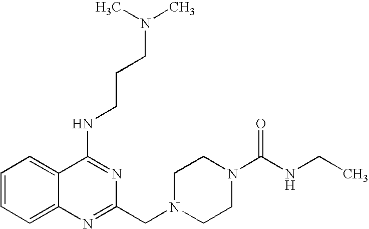 | 28-8 | 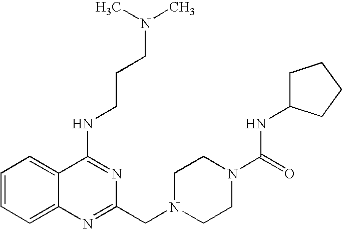 |
| 28-2 | 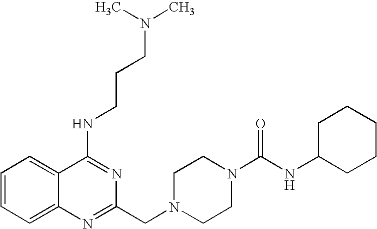 | 28-9 | 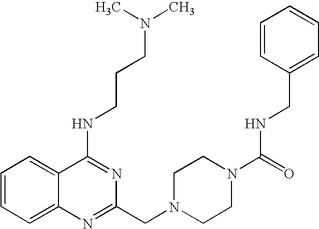 |
| 28-3 | 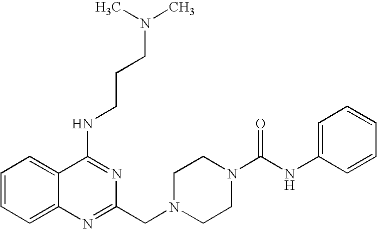 | 28-10 | 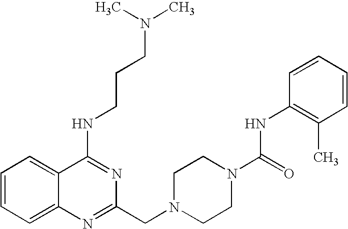 |
| 28-4 | 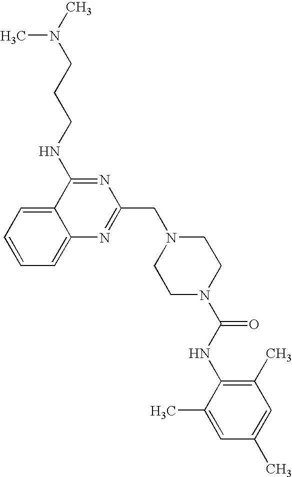 | 28-11 | 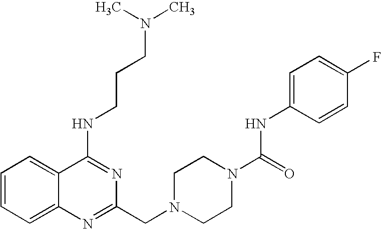 |
| 28-5 | 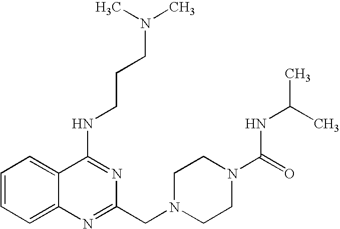 | 28-12 | 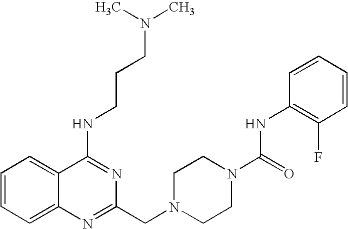 |
| 28-13 | 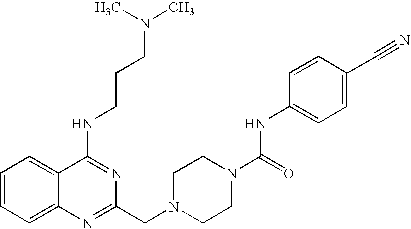 | 28-20 | 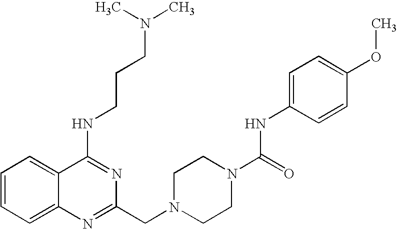 |
| 28-14 | 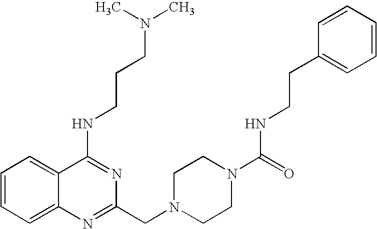 | 28-21 | 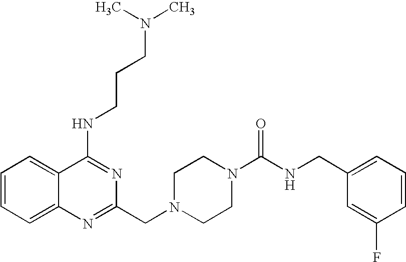 |
| 28-15 | 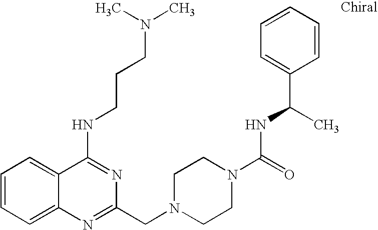 | 28-22 | 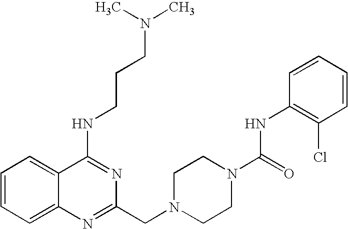 |
| 28-16 | 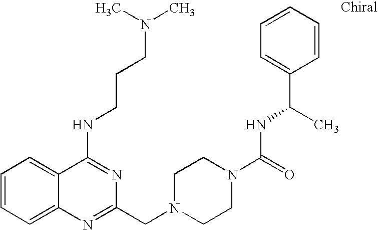 | 28-23 | 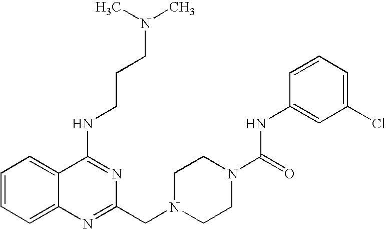 |
| 28-17 | 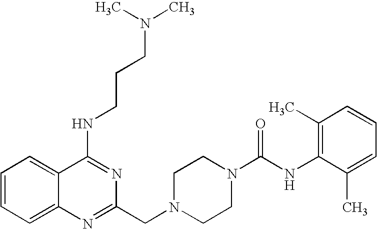 | 28-24 | 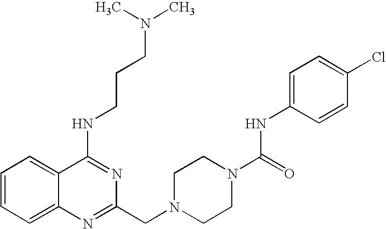 |
| 28-18 | 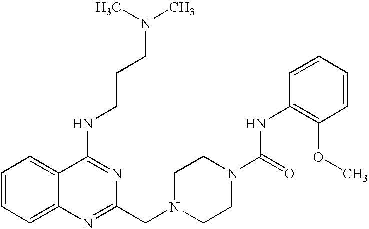 | 28-25 | 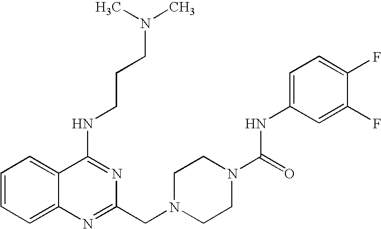 |
| 28-19 | 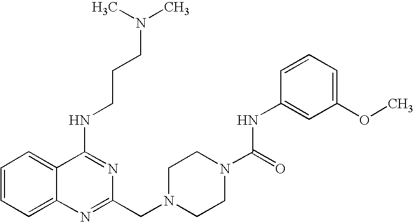 | 28-26 | 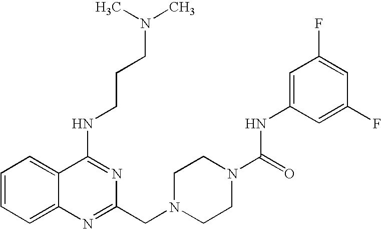 |
| 28-27 | 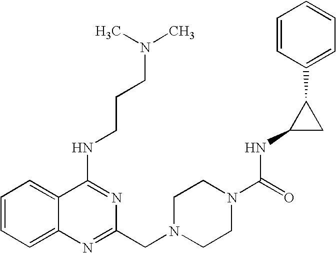 | 28-34 | 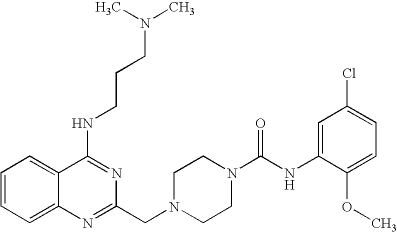 |
| 28-28 | 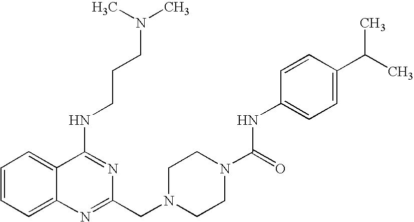 | 28-35 | 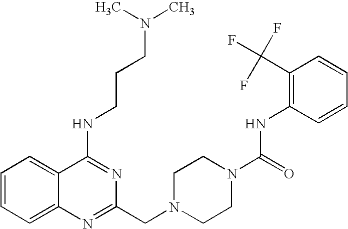 |
| 28-29 | 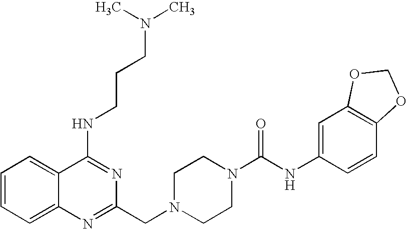 | 28-36 | 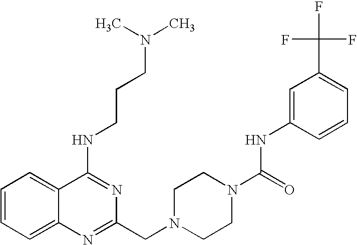 |
| 28-30 | 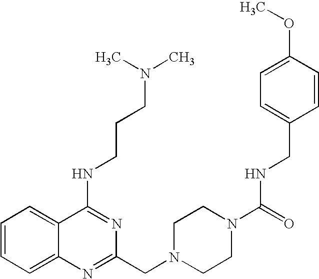 | 28-37 | 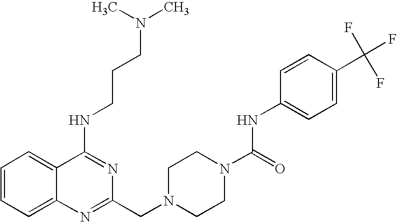 |
| 28-31 | 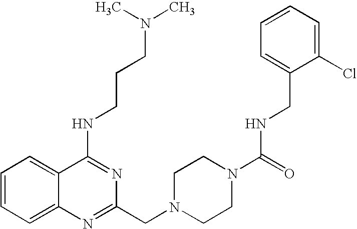 | 28-38 | 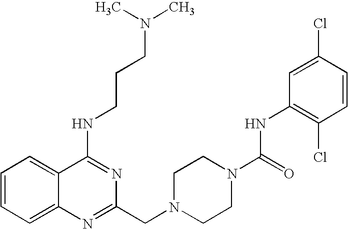 |
| 28-32 | 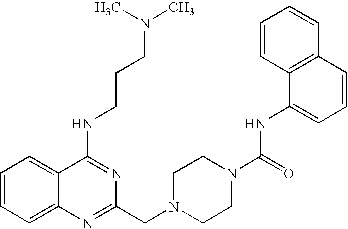 | 28-39 | 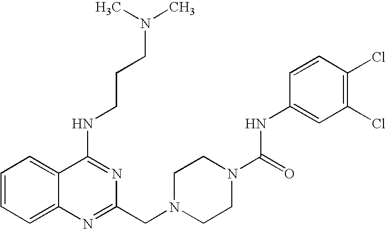 |
| 28-33 | 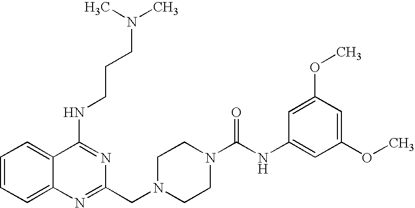 | 28-40 | 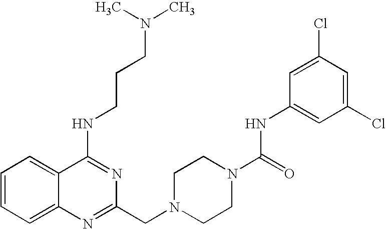 |
| 28-41 | 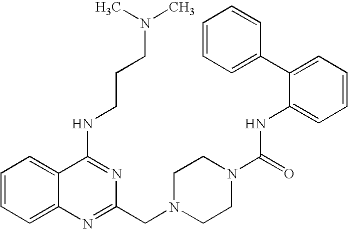 | 28-48 | 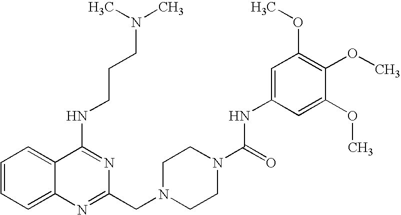 |
| 28-42 | 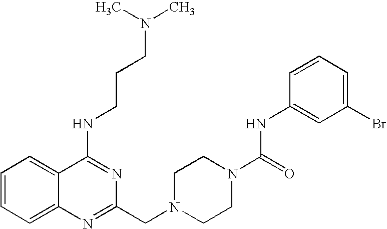 | 29-1 | 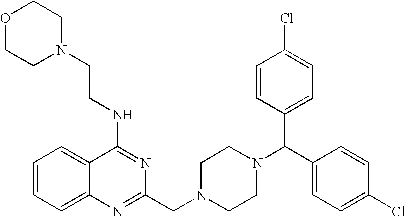 |
| 28-43 | 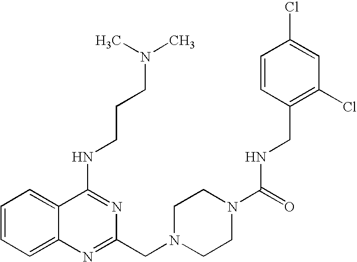 | 29-2 | 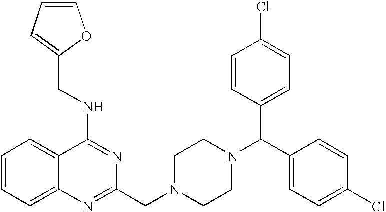 |
| 28-44 | 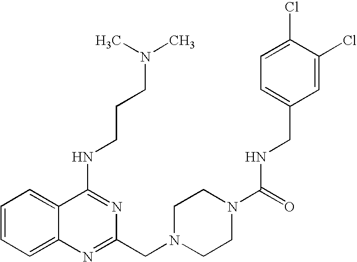 | 29-3 | 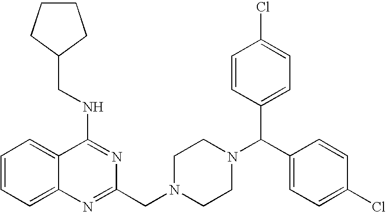 |
| 28-45 | 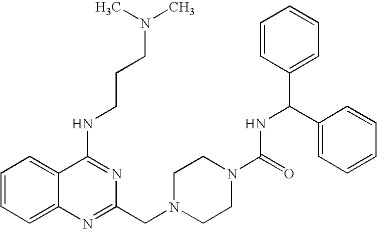 | 29-4 | 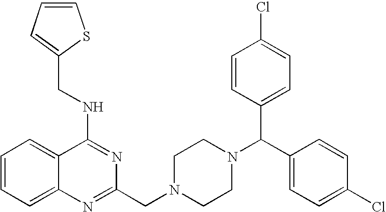 |
| 28-46 | 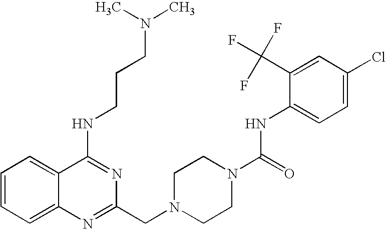 | 29-5 | 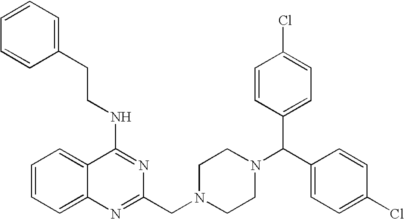 |
| 28-47 | 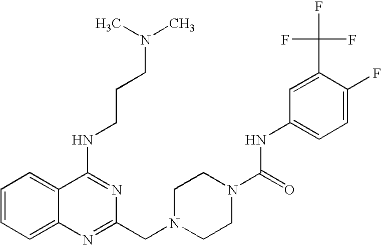 | 29-6 | 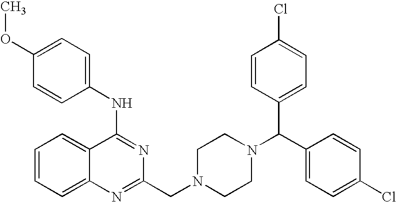 |
| 29-7 | 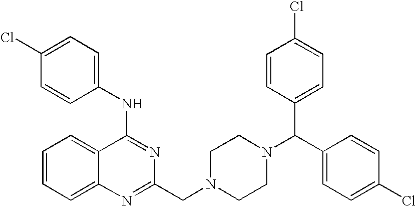 | 29-14 | 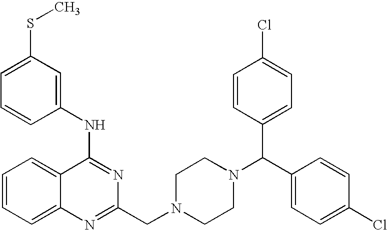 |
| 29-8 | 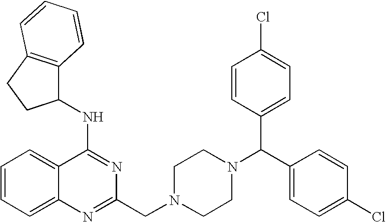 | 29-15 | 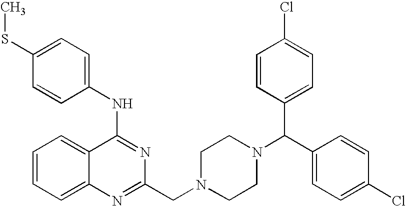 |
| 29-9 | 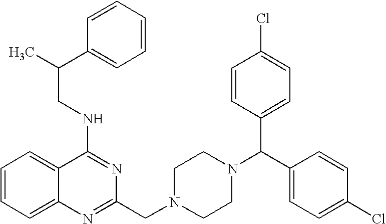 | 29-16 | 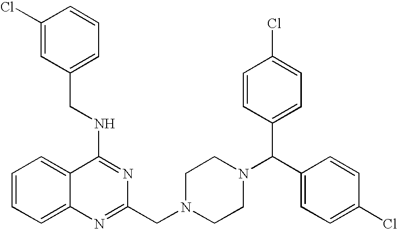 |
| 29-10 | 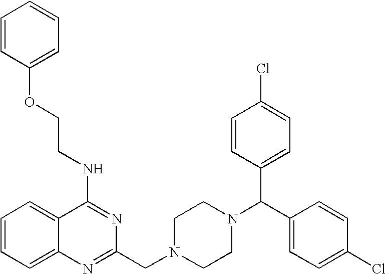 | 29-17 | 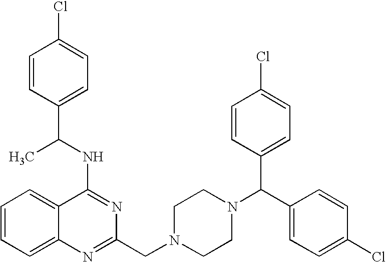 |
| 29-11 | 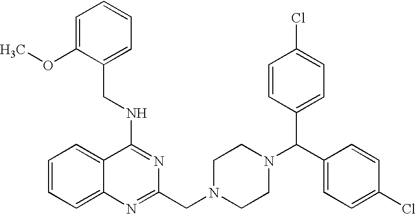 | 29-18 | 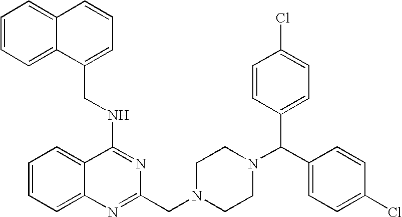 |
| 29-12 | 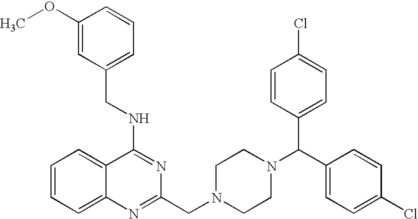 | 29-19 | 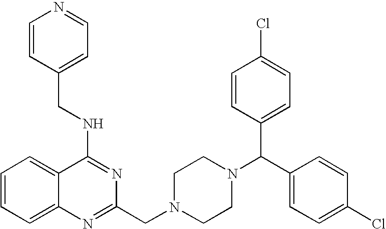 |
| 29-13 | 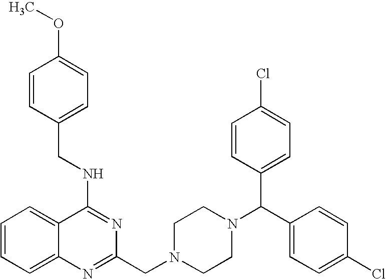 | 29-20 | 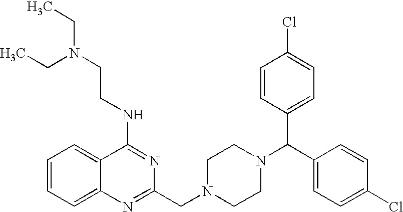 |
| 29-21 | 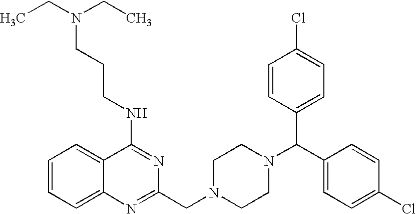 | 29-28 | 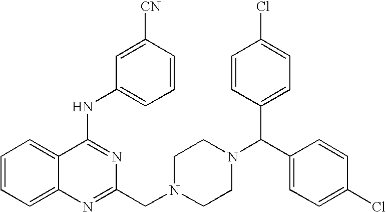 |
| 29-22 | 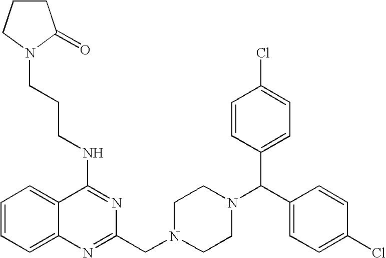 | 29-29 | 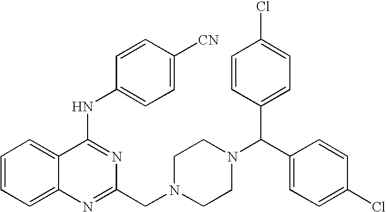 |
| 29-23 | 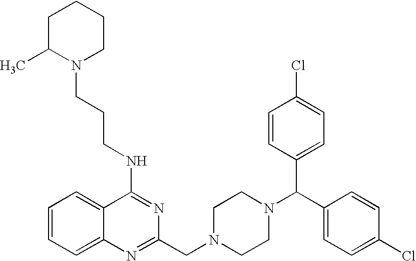 | 29-30 | 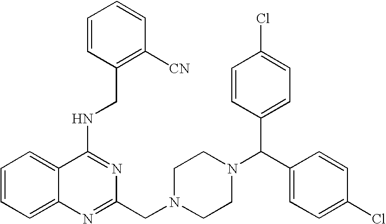 |
| 29-24 | 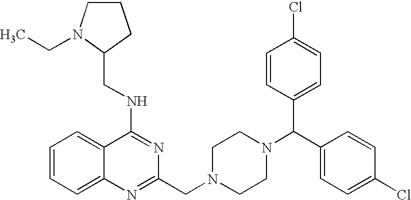 | 29-31 | 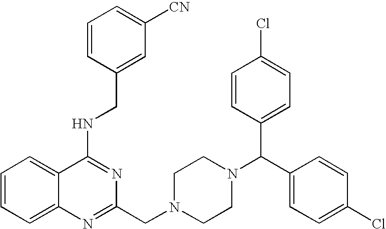 |
| 29-25 | 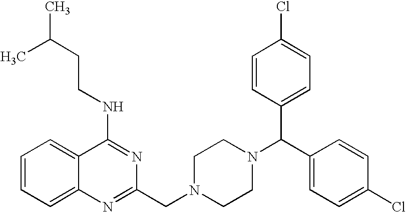 | 29-32 | 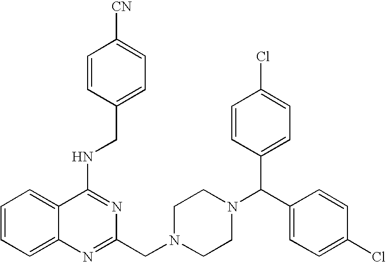 |
| 29-26 | 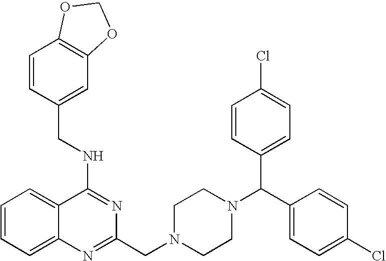 | 29-33 | 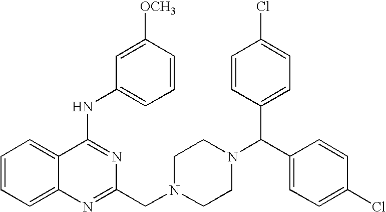 |
| 29-27 | 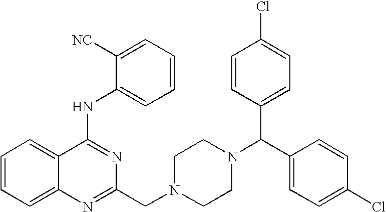 | 29-34 | 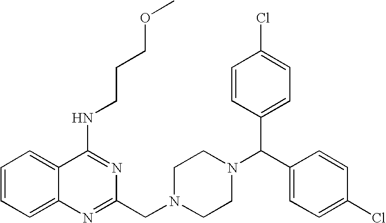 |
| 29-35 | 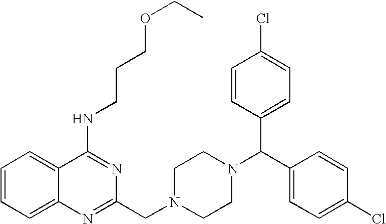 | 29-42 | 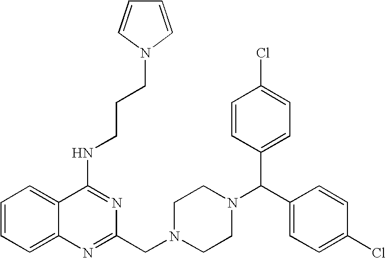 |
| 29-36 | 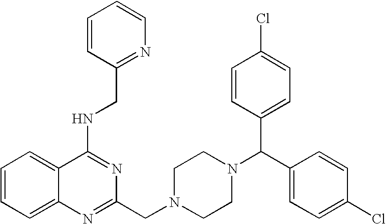 | 29-43 | 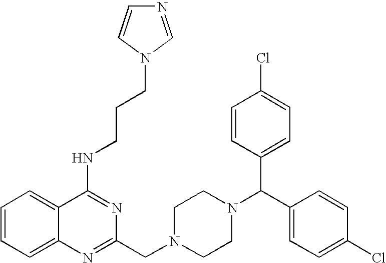 |
| 29-37 | 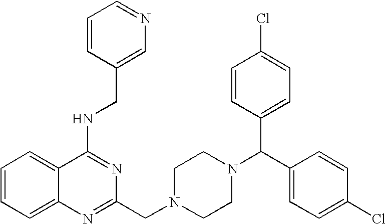 | 29-44 | 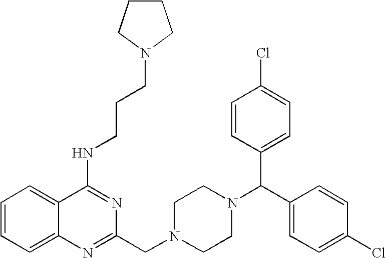 |
| 29-38 | 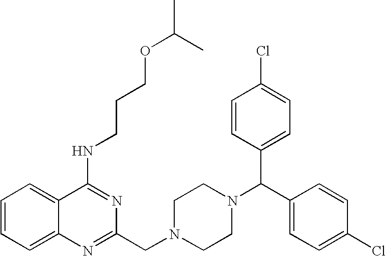 | 29-45 | 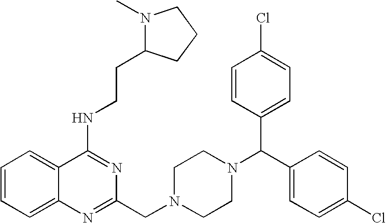 |
| 29-39 | 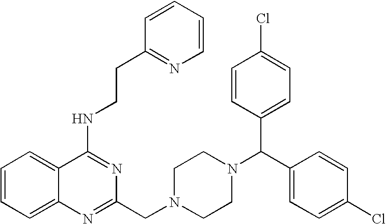 | 29-46 | 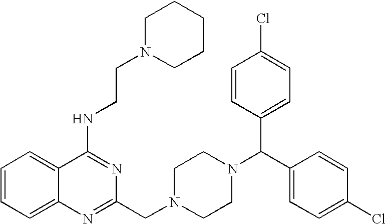 |
| 29-40 | 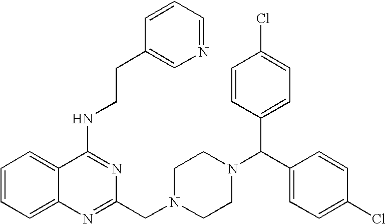 | 29-47 | 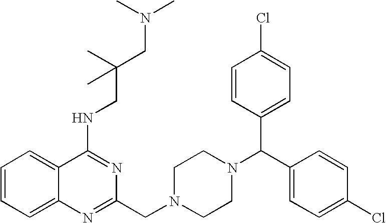 |
| 29-41 | 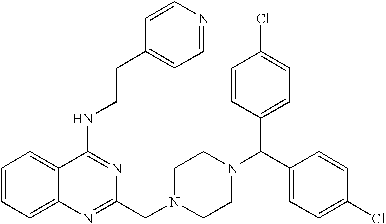 | 29-48 | 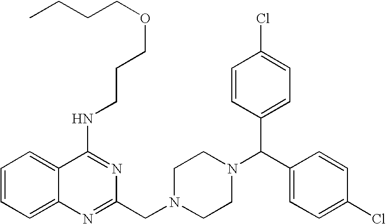 |
| 29-49 | 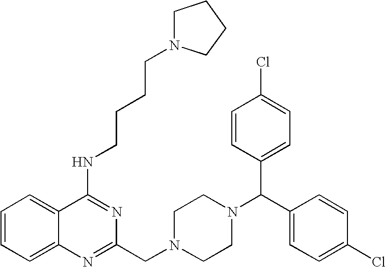 | 29-56 | 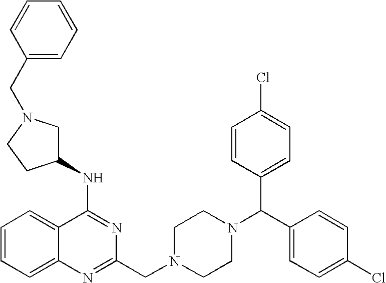 |
| 29-50 | 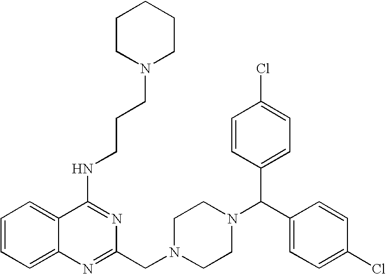 | 29-57 | 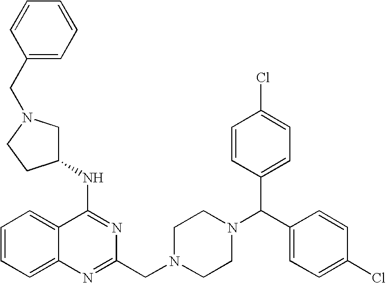 |
| 29-51 | 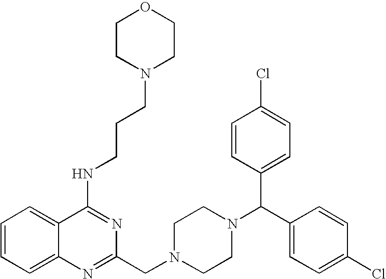 | 29-58 | 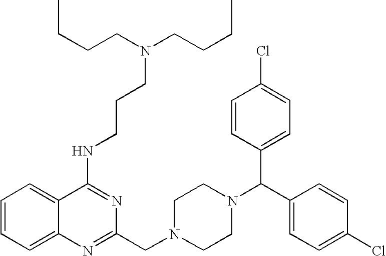 |
| 29-52 | 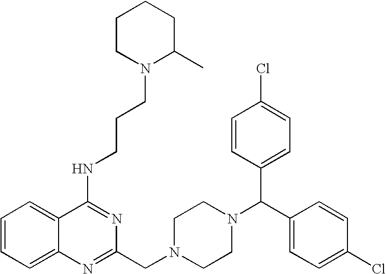 | 29-59 | 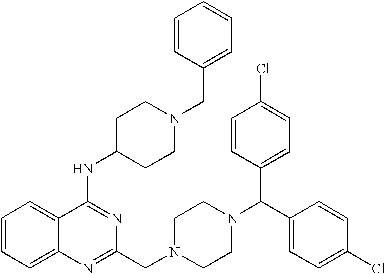 |
| 29-53 | 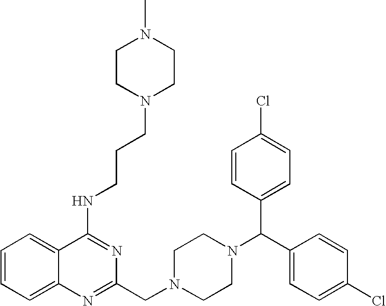 | 29-60 | 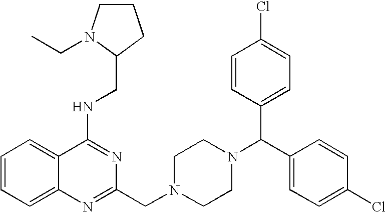 |
| 29-54 | 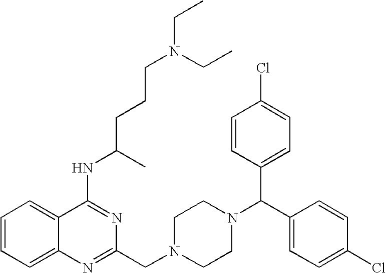 | 29-61 | 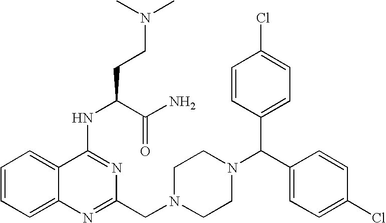 |
| 29-55 | 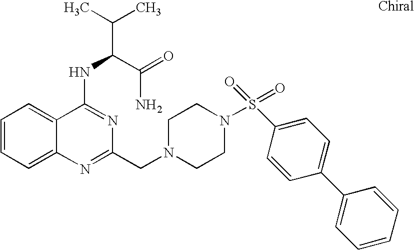 | 29-62 | 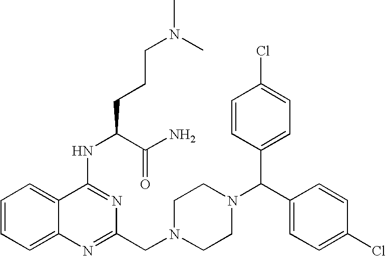 |
| 29-63 | 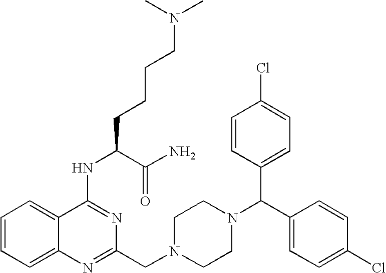 | 30-3 | 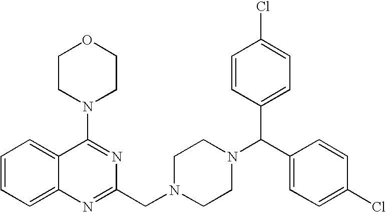 |
| 29-64 | 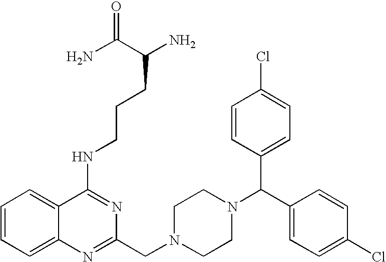 | 30-4 | 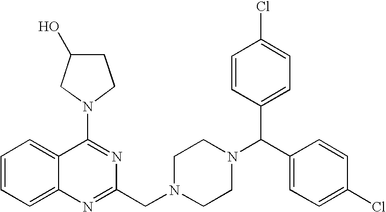 |
| 29-65 | 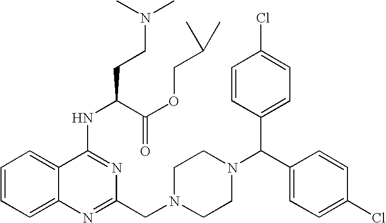 | 30-5 | 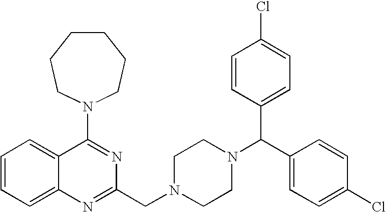 |
| 29-66 | 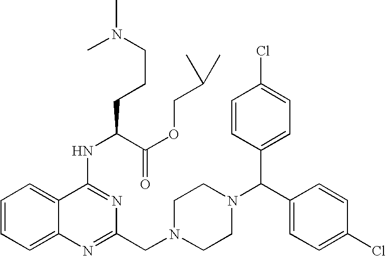 | 30-6 | 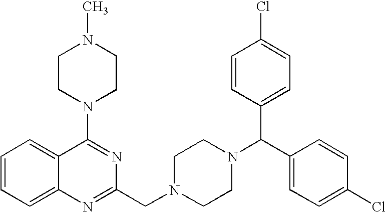 |
| 29-67 | 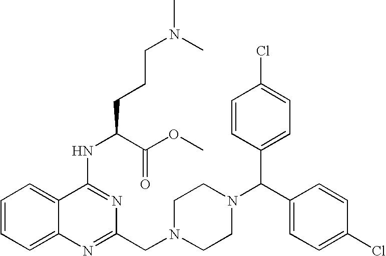 | 30-7 | 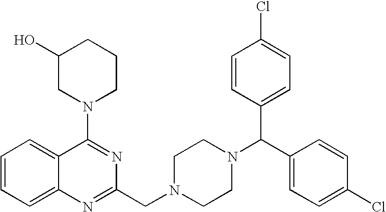 |
| 30-1 | 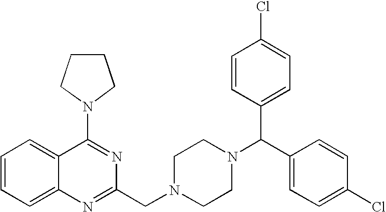 | 30-8 | 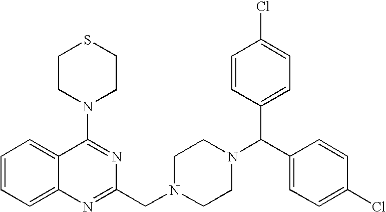 |
| 30-2 | 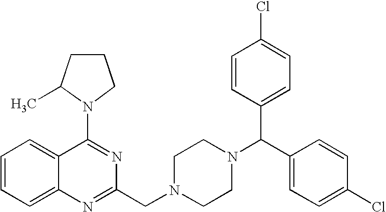 | 30-9 | 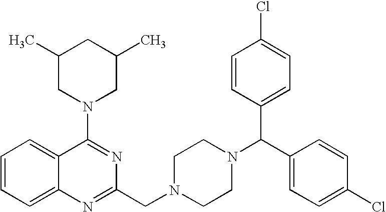 |
| 30-10 | 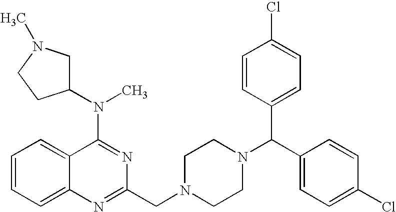 | 30-17 | 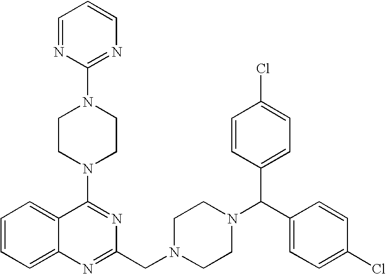 |
| 30-11 | 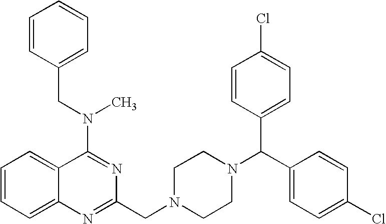 | 30-18 | 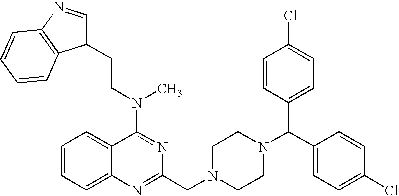 |
| 30-12 | 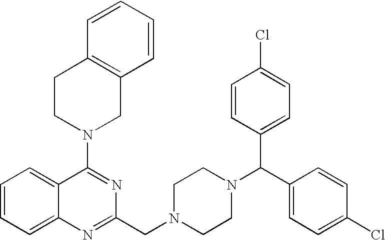 | 30-19 | 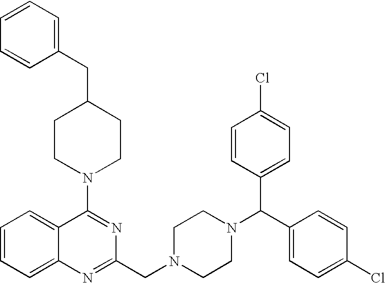 |
| 30-13 | 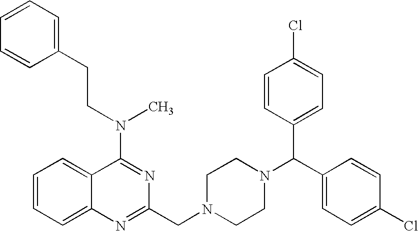 | 30-20 | 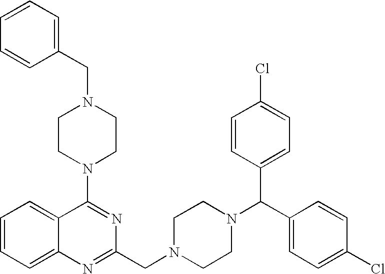 |
| 30-14 | 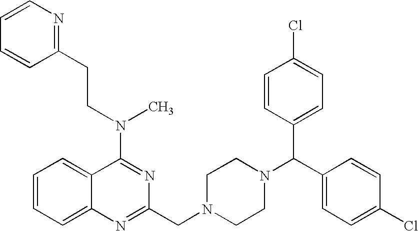 | 30-21 | 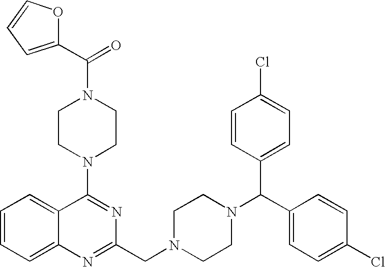 |
| 30-15 | 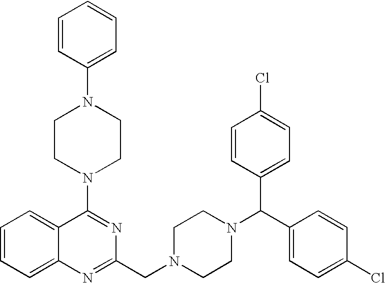 | 30-22 | 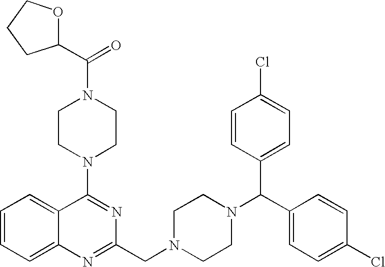 |
| 30-16 | 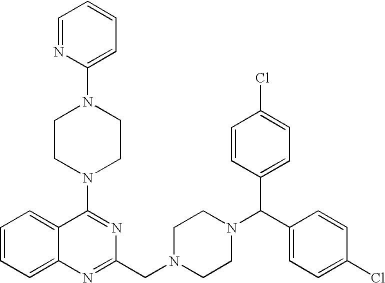 | 30-23 | 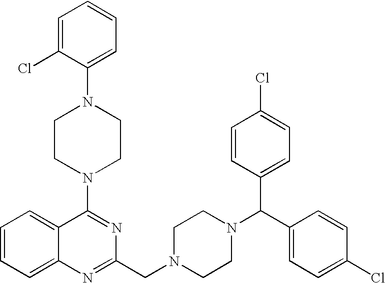 |
| 30-24 | 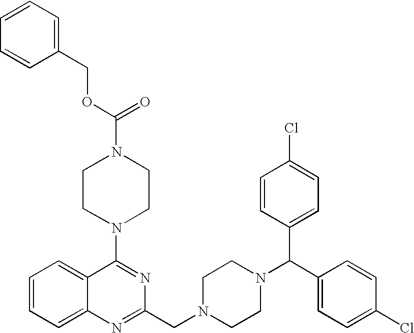 | 30-31 | 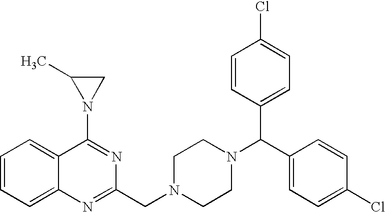 |
| 30-25 | 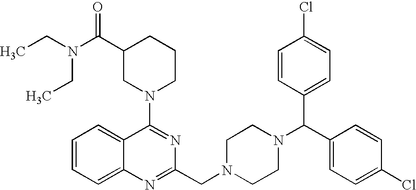 | 30-32 | 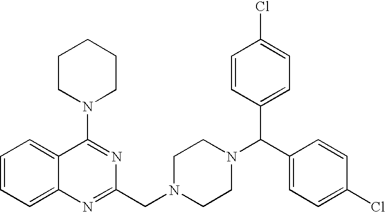 |
| 30-26 | 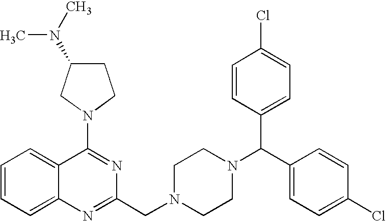 | 30-33 | 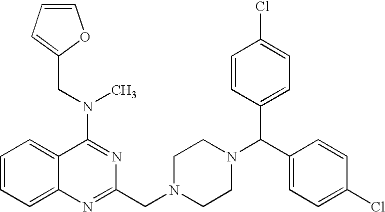 |
| 30-27 | 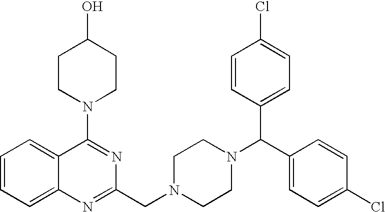 | 30-34 | 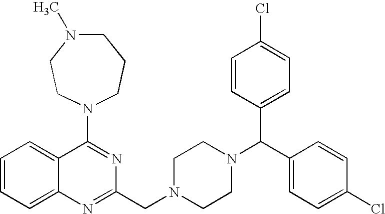 |
| 30-28 | 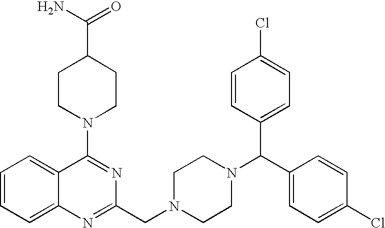 | 30-35 | 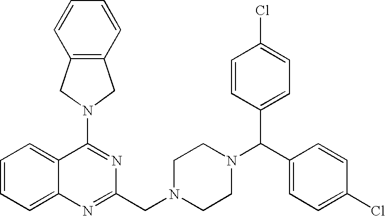 |
| 30-29 | 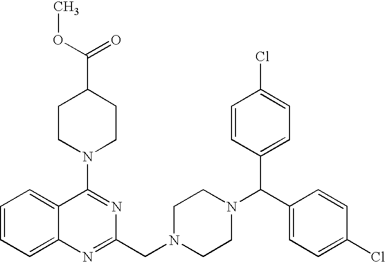 | 30-36 | 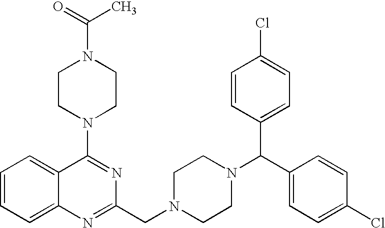 |
| 30-30 | 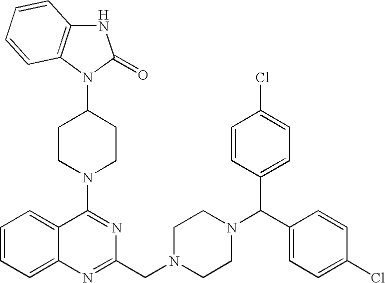 | 30-37 | 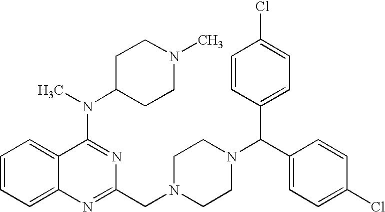 |
| 30-38 | 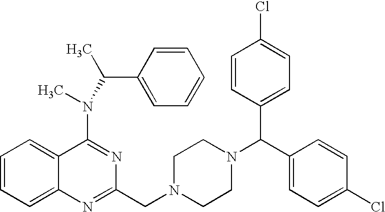 | 30-45 | 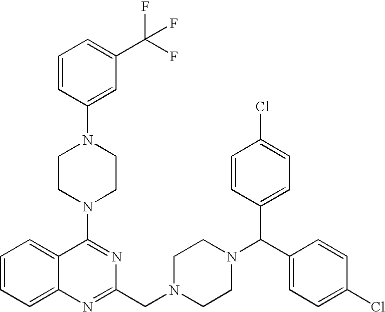 |
| 30-39 | 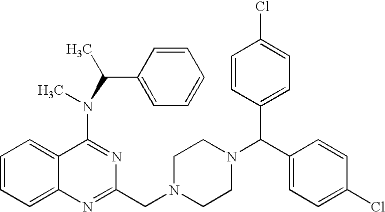 | 30-46 | 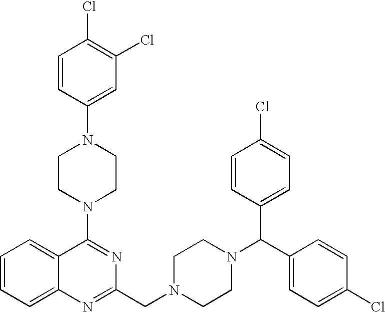 |
| 30-40 | 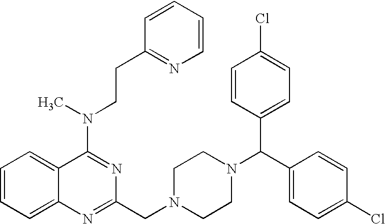 | 30-47 | 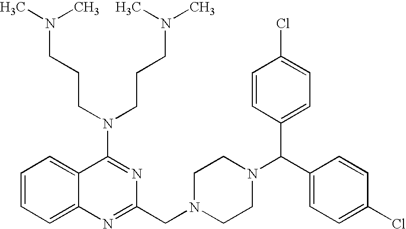 |
| 30-41 | 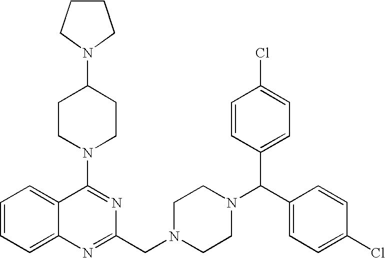 | 30-48 | 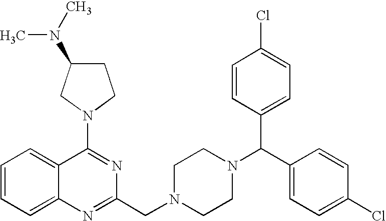 |
| 30-42 | 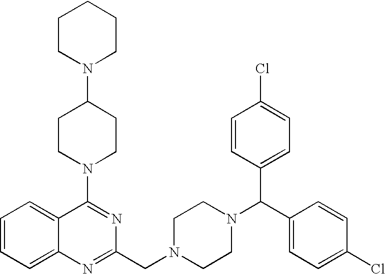 | 30-49 | 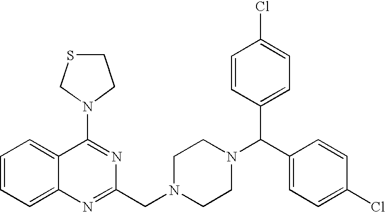 |
| 30-43 | 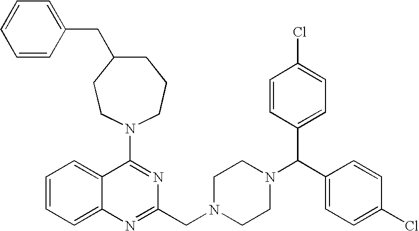 | 30-50 | 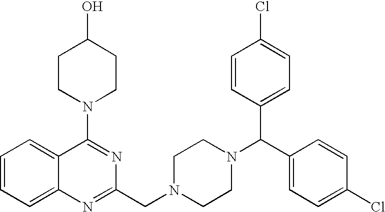 |
| 30-44 | 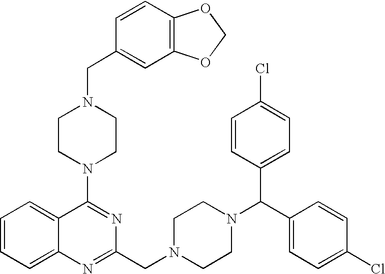 | 30-51 | 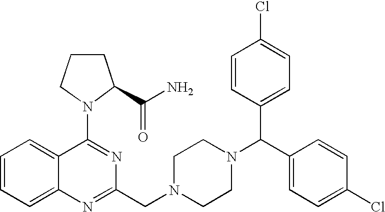 |
| 30-52 | 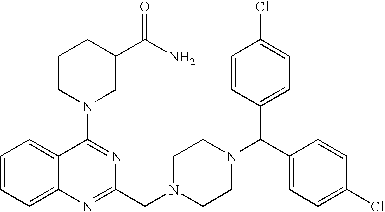 | 43 | 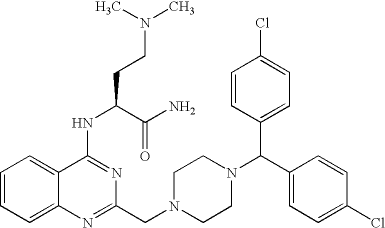 |
| 30-53 | 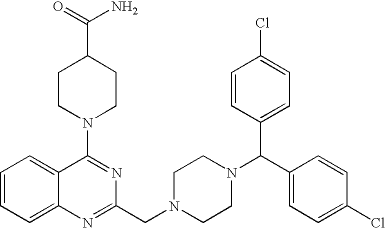 | 44 | 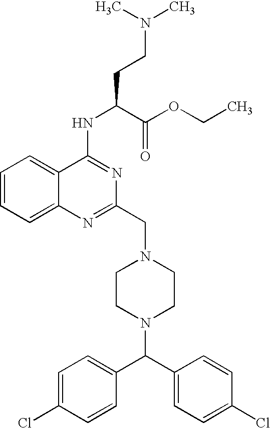 |
| 30-54 | 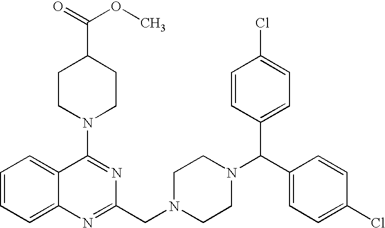 | 45 | 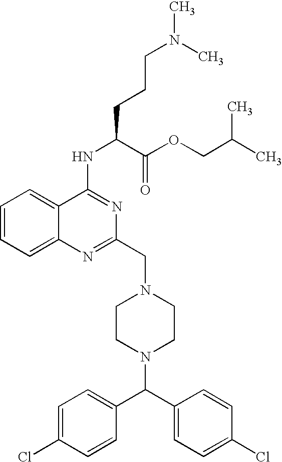 |
| 30-55 | 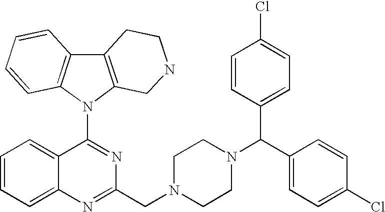 | 46 | 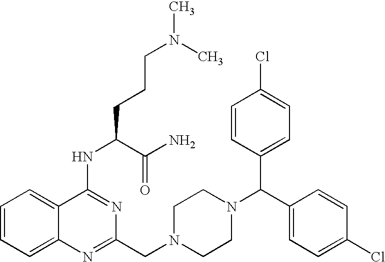 |
| 30-56 | 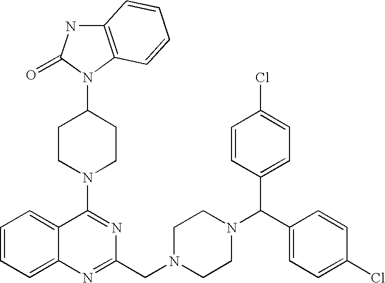 | 47 | 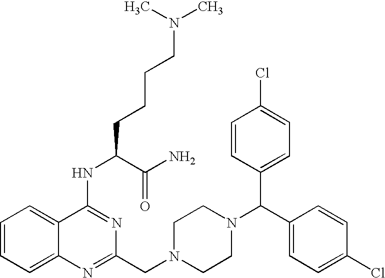 |
| 30-57 | 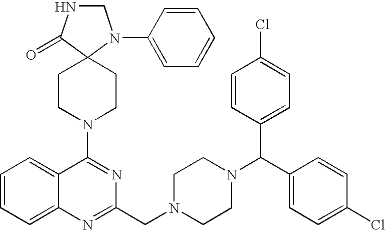 | 30-58 | 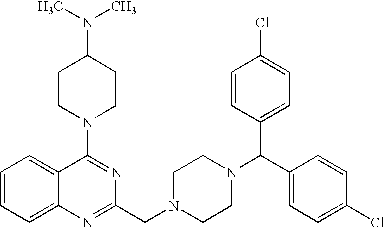 |

or a pharmaceutically acceptable salt, solvate or ester thereof.

In the above table of compounds, the compound # corresponds to the particular example # set forth in the “EXAMPLES” section below where the preparation of such compound is shown. Where a compound has two numbers separated by a dash (−), the first number represents the example # where the preparation of the compound is shown, and the second number designates an arbitrary number for the particular compound. Thus compound #11-1 indicates that this a compound whose preparation is shown in Example 10). Similarly #11-2 indicates a different compound whose preparation is also shown in Example 11. In another preferred embodiment, the compound of formula I is selected from the group consisting of

| COM- | | | |
|---|---|---|---|
| POUND # | STRUCTURE | COMPOUND # | STRUCTURE |
| 1 | 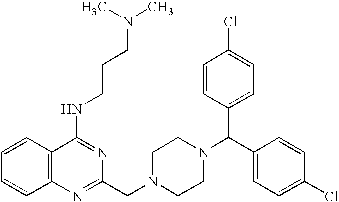 | 20-29 | 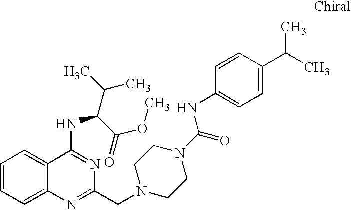 |
| 11-1 | 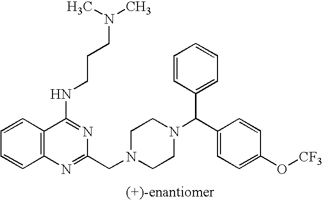 | 20-46 | 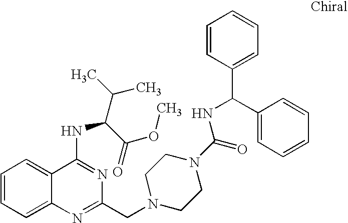 |
| 11-2 | 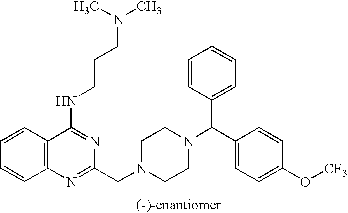 | 25-29 | 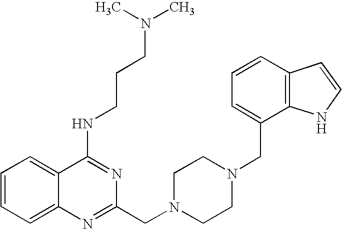 |
| 25-36 | 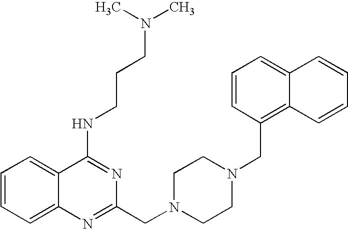 | 30-3 | 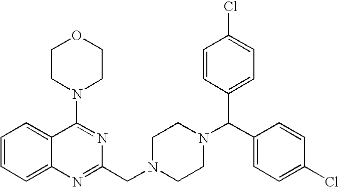 |
| 27-73 | 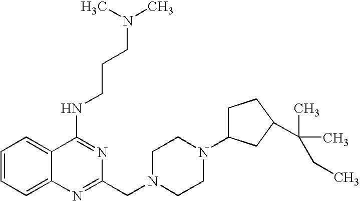 | 30-4 | 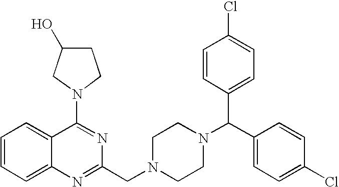 |
| 26-90 | 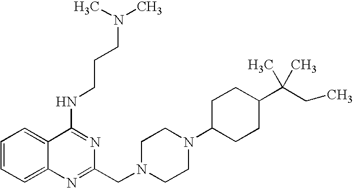 | 30-11 | 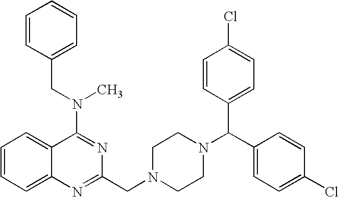 |
| 28-4 | 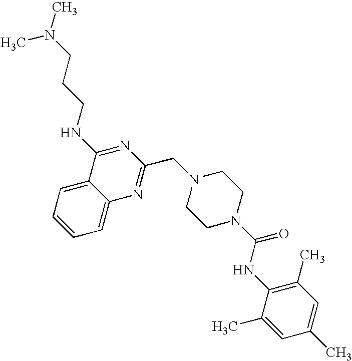 | 30-12 | 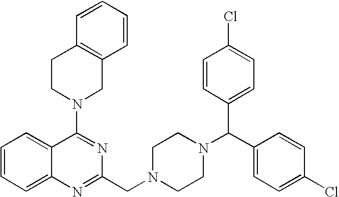 |
| 28-13 | 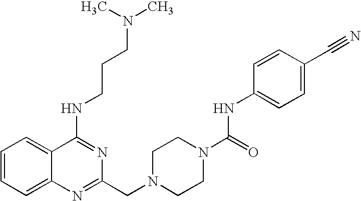 | 30-14 | 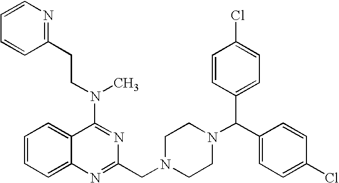 |
| 28-20 | 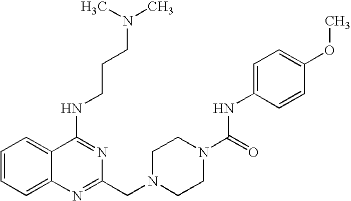 | 30-15 | 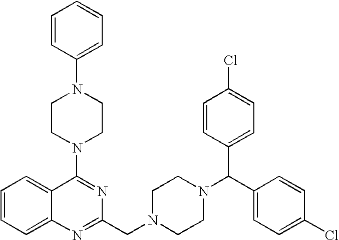 |
| 28-39 | 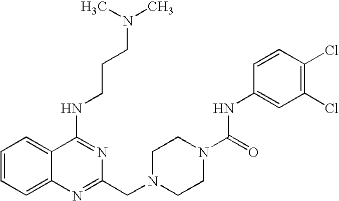 | 30-17 | 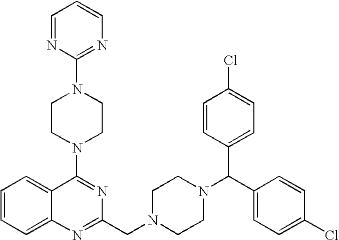 |
| 30-22 | 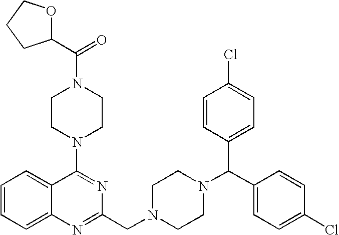 | 30-29 | 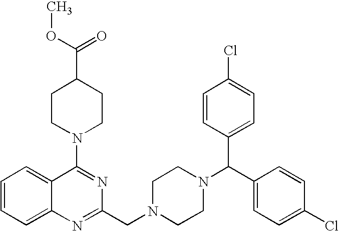 |
| 30-25 | 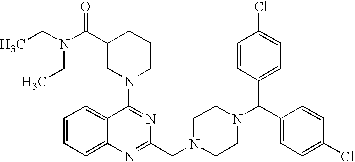 | 30-30 | 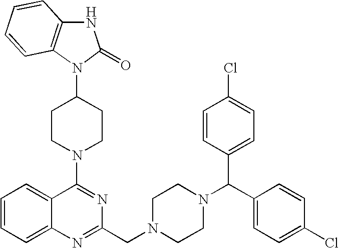 |
| 30-26 | 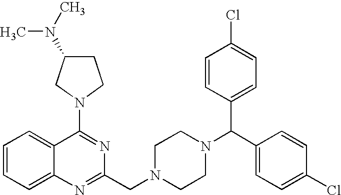 | 43 | 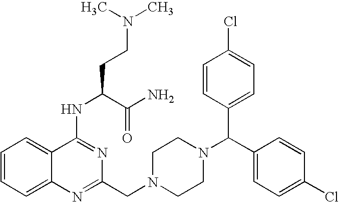 |
| 30-27 | 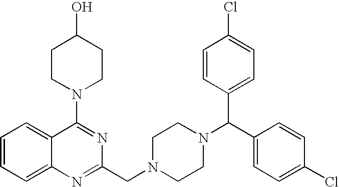 | 44 | 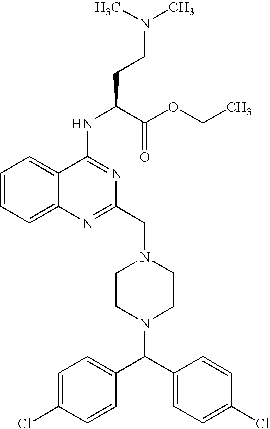 |
| 30-28 | 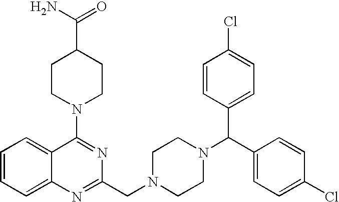 | 45 | 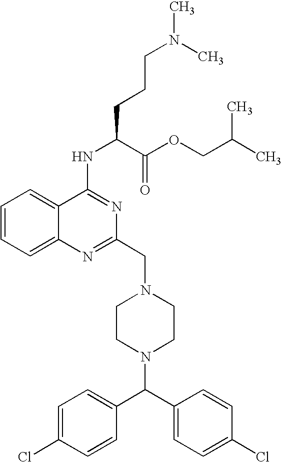 |
| 46 | 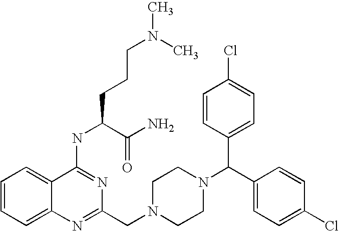 | 47 | 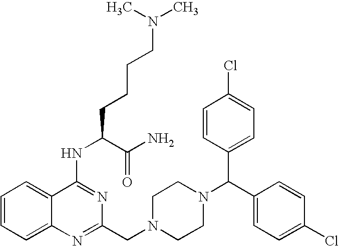 |

or a pharmaceutically acceptable salt, solvate or ester thereof.

In another more preferred embodiment, the compound of formula I is selected from the group consisting of

| COMPOUND | | COMPOUND | |
|---|---|---|---|
| # | STRUCTURE | # | STRUCTURE |
| 1 | 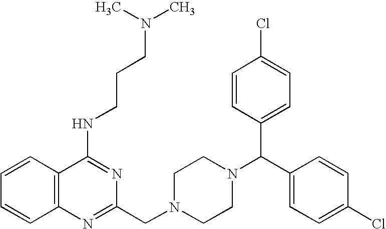 | 11-2 | 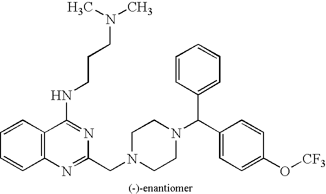 |
| 11-1 | 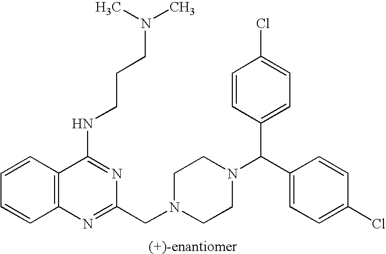 | 20-29 | 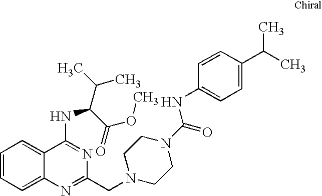 |
| 20-46 | 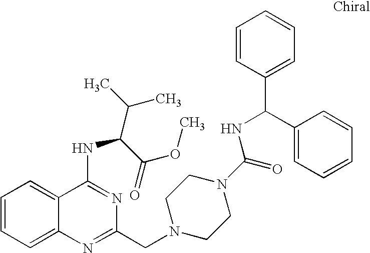 | 30-30 | 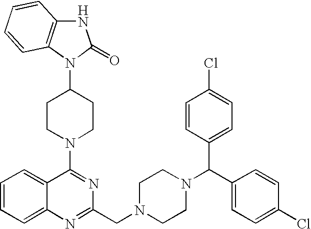 |
| 25-29 | 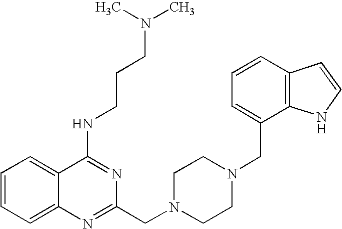 | 43 | 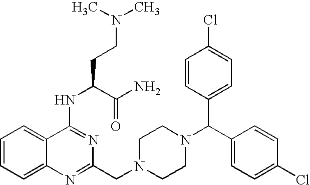 |
| 25-36 | 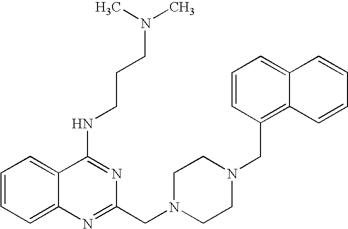 | 44 | 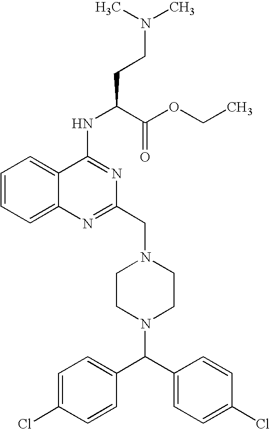 |
| 25-90 | 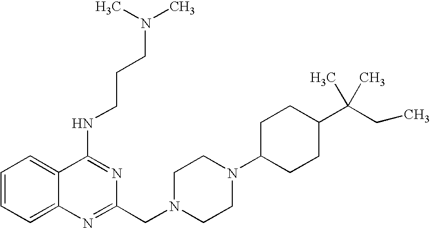 | 45 | 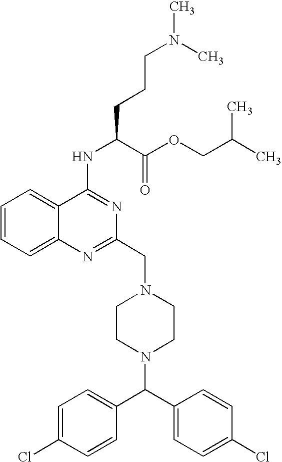 |
| 28-20 | 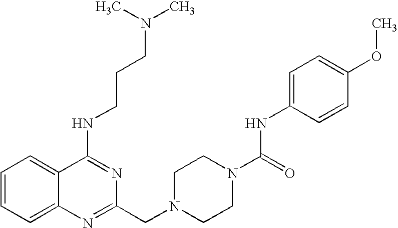 | 46 | 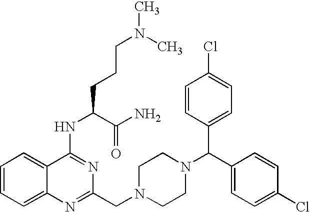 |
| 28-39 | 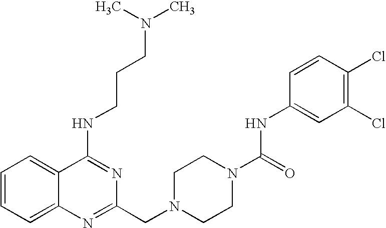 | 47 | 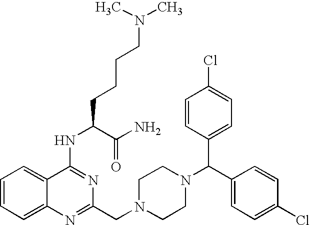 |

or a pharmaceutically acceptable salt, solvate or ester thereof.

In another most preferred embodiment, the compound of formula I is selected from the group consisting of

| COMPOUND # | STRUCTURE |
|---|---|
| 1 | 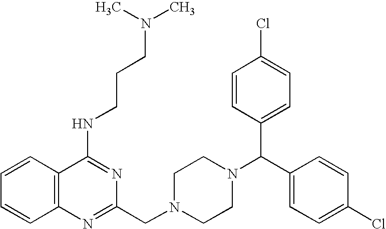 |
| 11-1 | 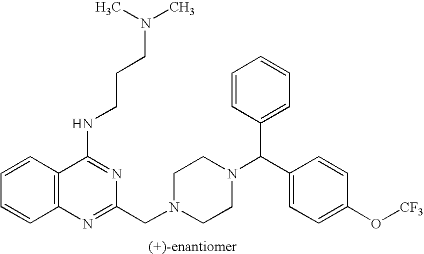 |
| 11-2 | 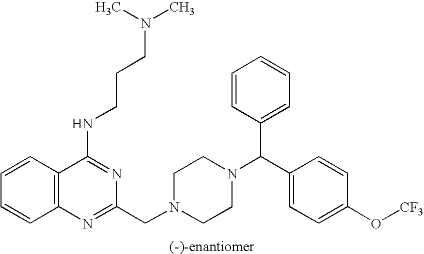 |
| 25-36 | 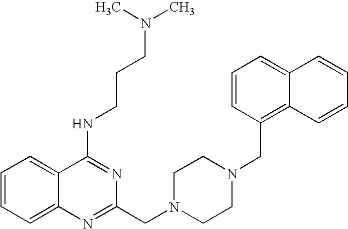 |
| 25-90 | 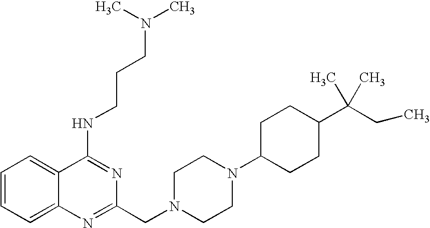 |

or a pharmaceutically acceptable salt, solvate or ester thereof.

In another embodiment, X in formula I is OR⁵; and m is 0 or 1; wherein R⁵ is as set forth above for formula I.

In another embodiment, X in formula I is OR⁵ wherein R⁵ is as set forth above for formula I; m is 0 or 1; and R¹ and R² are both hydrogen.

In another embodiment, X in formula I is OR⁵ wherein R⁵ is as set forth above for formula I; m is 0; and R¹ and R² are both hydrogen.

In another embodiment, X in formula I is OR⁵; m is 0 or 1; and R¹ and R² are both hydrogen; wherein R⁵ is alkyl; wherein said alkyl is substituted with one moiety selected from the group consisting of heterocyclic, (alkyl)₂-amino, and alkoxy; wherein each of the alkyl alteratives of the aforesaid (alkyl)₂-amino and alkoxy moieties may optionally be substituted with an (alkyl)₂-amino radical.

In another embodiment, X in formula I is OR⁵; m is 0 or 1; R¹ and R² are both hydrogen; R⁵ is alkyl; wherein said alkyl is substituted with one moiety selected from the group consisting of heterocyclic, (alkyl)₂-amino, and alkoxy; wherein each of the alkyl alteratives of the aforesaid (alkyl)₂-amino and alkoxy moieties may optionally be substituted with an (alkyl)₂-amino radical; and R⁴ is alkyl; wherein said alkyl is substituted with two phenyl substituents; wherein each phenyl substituent is substituted with two halo moieties.

In another embodiment, the compound of formula I is selected from the group consisting of

| EXAMPLE | STRUCTURE |
|---|---|
| 31-1 | 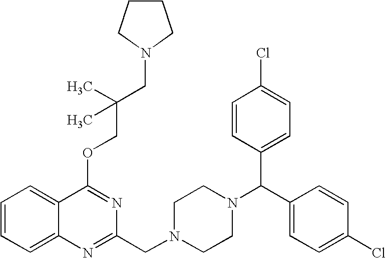 |
| 31-2 | 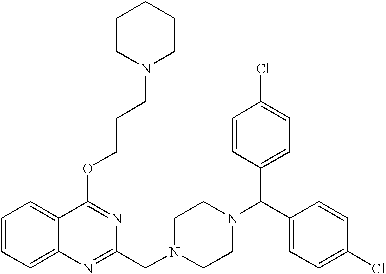 |
| 31-3 | 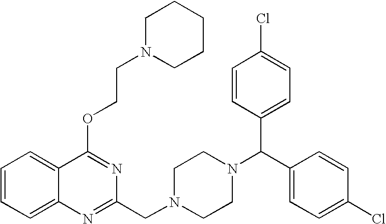 |
| 31-4 | 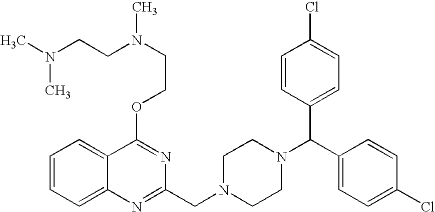 |
| 31-5 | 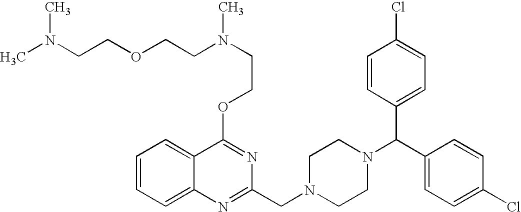 |
| 31-6 | 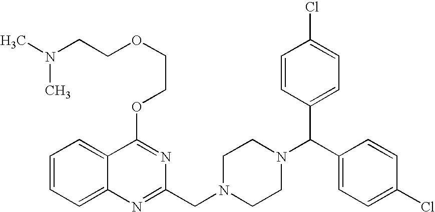 |

or a pharmaceutically acceptable salt, solvate or ester thereof.

In other embodiments, the present invention provides processes for producing such compounds, pharmaceutical formulations or compositions comprising one or more of such compounds, and methods of treating or preventing one or more conditions or diseases associated with p53 mutant activity such as those discussed in detail below.

As used above, and throughout the specification, the following terms, unless otherwise indicated, shall be understood to have the following meanings:

“Subject” includes both mammals and non-mammalian animals.

“Mammal” includes humans and other mammalian animals.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

The term “optionally substituted” means optional substitution with the specified groups, radicals or moieties. It should be noted that any atom with unsatisfied valences in the text, schemes, examples and tables herein is assumed to have the hydrogen atom(s) to satisfy the valences.

The terms “substituent”, “moiety” and “radical” have specific and distinct meanings as used herein and represent a hierarchy in the use of such terms. The hierarchy used generally is “substituent”→“moiety”→“radical”, starting out with “substituent” and ending with “radical” while describing the branching out of various groups. Thus, for example, a specific R group will be described as being selected from a group of specified substituents. The substituents will then be described as having certain “moieties”, and those moieties will be described as having certain “radicals”. Thus an “alkyl substituent” as used herein is differentiated from an “alkyl moiety” which in turn is differentiated from an “alkyl radical”. Such use of terminology is generally adhered to consistently throughout the specification for preservation of proper antecedent basis.

The term “aryl alternative” refers to a certain “moiety” or “radical” wherein said “moiety” or “radical” contains an aryl group as part a larger group. For example, in the phrase, “ . . . substituted by one to four moieties selected from alkyl, alkoxy, perfluoroalkyl, aryloxy, aryl-O—(C═O)—NH, aryl-S(O)₂NH, and aryl-HN—(C═O)—O—, wherein each of the aforesaid moieties containing an aryl alternative may optionally be independently substituted by one or two radicals selected from the group consisting of halo, alkyl and cyano”, the term “aforesaid moieties containing an aryl alternative” refers to the aryloxy, aryl-O—(C═O)—NH, aryl-S(O)₂NH, and aryl-HN—(C═O)—O— moieties, and it is the aryl group within these aryloxy, aryl-O—(C═O)—NH, aryl-S(O)₂NH, and aryl-HN—(C═O)—O— moieties that may be substituted with the halo, alkyl and cyano radicals.

The following definitions apply regardless of whether a term is used by itself or in combination with other terms, unless otherwise indicated. Therefore, the definition of “alkyl” applies to “alkyl” as well as the “alkyl” portions of “hydroxyalkyl”, “haloalkyl”, “alkoxy”, etc.

As used herein, the term “alkyl” means an aliphatic hydrocarbon group which may be straight or branched and comprising about 1 to about 20 carbon atoms in the chain. Preferred alkyl groups contain about 1 to about 12 carbon atoms in the chain. More preferred alkyl groups contain about 1 to about 6 carbon atoms in the chain. Branched means that one or more lower alkyl groups such as methyl, ethyl or propyl, are attached to a linear alkyl chain. “Lower alkyl” means a group having about 1 to about 6 carbon atoms in the chain which may be straight or branched. The alkyl group may be substituted with one or more substituents independently selected from the group consisting of halo, alkyl, aryl, cycloalkyl, cyano, hydroxy, alkoxy, amino, —NH(alkyl), —NH(cycloalkyl), —N(alkyl)₂, carboxy, —C(O)O-alkyl and —S(alkyl), wherein said alkyl, cycloalkyl and aryl are unsubstituted. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl. Whenever applicable, the term “alkyl” also includes a divalent alkyl, i.e., an “alkylene” group, obtained by removal of a hydrogen atom from an alkyl group. Examples of alkylene groups include methylene (—CH₂—), ethylene (—CH₂CH₂—), propylene (—C₃H₆—) and the like including where applicable both straight chain and branched structures.

“Alkenyl” means an aliphatic hydrocarbon group containing at least one carbon-carbon double bond and which may be straight or branched and comprising about 2 to about 15 carbon atoms in the chain. Preferred alkenyl groups have about 2 to about 12 carbon atoms in the chain; and more preferably about 2 to about 6 carbon atoms in the chain. Branched means that one or more lower alkyl groups such as methyl, ethyl or propyl, are attached to a linear alkenyl chain. “Lower alkenyl” means about 2 to about 6 carbon atoms in the chain which may be straight or branched. The alkenyl group may be substituted with one or more substituents independently selected from the group consisting of halo, alkyl, aryl, cycloalkyl, cyano, alkoxy and —S(alkyl), wherein said alkyl, cycloalkyl and aryl are unsubstituted. Non-limiting examples of suitable alkenyl groups include ethenyl, propenyl, n-butenyl, 3-methylbut-2-enyl, n-pentenyl, octenyl and decenyl.

“Alkynyl” means an aliphatic hydrocarbon group containing at least one carbon-carbon triple bond and which may be straight or branched and comprising about 2 to about 15 carbon atoms in the chain. Preferred alkynyl groups have about 2 to about 12 carbon atoms in the chain; and more preferably about 2 to about 4 carbon atoms in the chain. Branched means that one or more lower alkyl groups such as methyl, ethyl or propyl, are attached to a linear alkynyl chain. “Lower alkynyl” means about 2 to about 6 carbon atoms in the chain which may be straight or branched. Non-limiting examples of suitable alkynyl groups include ethynyl, propynyl, 2-butynyl, 3-methylbutynyl, n-pentynyl, and decynyl. The alkynyl group may be substituted with one or more substituents being independently selected from the group consisting of alkyl, aryl and cycloalkyl, wherein said alkyl, cycloalkyl and aryl are unsubstituted.

“Alkoxy” means an alkyl-O— group in which the alkyl group is as previously described. Useful alkoxy groups can comprise 1 to about 12 carbon atoms, preferably 1 to about 6 carbon atoms. Non-limiting examples of suitable alkoxy groups include methoxy, ethoxy and isopropoxy. The alkyl group of the alkoxy is linked to an adjacent moiety through the ether oxygen.

The term “perhaloalkyl” means, unless otherwise stated, alkyl substituted with (2m′+1) halogen atoms, where m′ is the total number of carbon atoms in the alkyl group. For example, the term “perhaloalkyl” includes trifluoromethyl, pentachloroethyl, 1,1,1-trifluoro-2-bromo-2-chloroethyl, and the like.

The term “perhaloalkoxy” means, unless otherwise stated, alkyloxy (i.e., alkoxy) substituted with (2m′+1) halogen atoms, where m′ is the total number of carbon atoms in the alkoxy group. For example, the term “perhaloalkoxy” includes trifluoromethoxy, pentachloroethoxy, 1,1,1-trifluoro-2-bromo-2-chloroethoxy, and the like.

“Aryl” means an aromatic monocyclic or multicyclic ring system comprising about 5 to about 14 carbon atoms, preferably about 6 to about 10 carbon atoms. The aryl group can be substituted with one or more “ring system substituents” which may be the same or different, and are as defined herein. Non-limiting examples of suitable aryl groups include phenyl and naphthyl. Also included within the scope of the term “aryl”, as used herein, is a group in which an aromatic hydrocarbon ring is fused to one or more non-aromatic carbocyclic or heteroatom-containing rings, such as in an indanyl, phenanthridinyl or tetrahydronaphthyl, where the radical or point of attachment is on the aromatic hydrocarbon ring.

“Aralkyl” or “arylalkyl” means an alkyl group substituted with an aryl group in which the aryl and alkyl are as previously described. Preferred aralkyls comprise a lower alkyl group. Non-limiting examples of suitable aralkyl groups include benzyl, phenethyl and naphthlenylmethyl. The aralkyl is linked to an adjacent moiety through the alkylene group.

“Cycloalkyl” means a non-aromatic mono- or multicyclic hydrocarbon ring system comprising about 3 to about 12 carbon atoms, preferably about 5 to about 10 carbon atoms. Preferred cycloalkyl rings contain about 5 to about 7 ring atoms. The cycloalkyl can be substituted with one or more “ring system substituents” which may be the same or different, and are as defined below. Non-limiting examples of suitable monocyclic cycloalkyls include cyclopropyl, cyclobutyl, cyclopentyl, cyclohexyl and the like. Non-limiting examples of suitable multicyclic cycloalkyls include 1-decalinyl, norbornyl, adamantyl and the like. A cycloalkyl may be fully saturated or may contain one or more units of unsaturation but is not aromatic. The term “cycloalkyl” also includes hydrocarbon rings that are fused to one or more aromatic rings where the radical or point of attachment is on the non-aromatic ring.

“Halo” or halogen refers to fluorine, chlorine, bromine or iodine radicals. Preferred are fluorine, chlorine and bromine

“Heteroaryl” means a monocyclic or multicyclic aromatic ring system of about 5 to about 14 ring atoms, preferably about 5 to about 10 ring atoms, in which one or more of the atoms in the ring system is/are atoms other than carbon, for example nitrogen, oxygen or sulfur. Preferred heteroaryls contain about 5 to about 6 ring atoms. The “heteroaryl” can be optionally substituted with one or more “ring system substituents” which may be the same or different, and are as defined herein. The prefix aza, oxa or thia before the heteroaryl root name means that at least a nitrogen, oxygen or sulfur atom respectively, is present as a ring atom. A nitrogen atom of a heteroaryl can be oxidized to form the corresponding N-oxide. All regioisomers are contemplated, e.g., 2-pyridyl, 3-pyridyl and 4-pyridyl. Examples of useful 6-membered heteroaryl groups include pyridyl, pyrimidinyl, pyrazinyl, pyridazinyl and the like and the N-oxides thereof. Examples of useful 5-membered heteroaryl rings include furyl, thienyl, pyrrolyl, thiazolyl, isothiazolyl, imidazolyl, pyrazolyl and isoxazolyl. Useful bicyclic groups are benzo-fused ring systems derived from the heteroaryl groups named above, e.g., quinolyl, phthalazinyl, quinazolinyl, benzofuranyl, benzothienyl and indolyl. Also included within the scope of the term “heteroaryl” is a group in which a heteroaromatic ring is fused to one or more aromatic or non-aromatic rings where the radical or point of attachment is on the heteroaromatic ring. The term “heteroaryl” also refers to partially saturated heteroaryl moieties such as, for example, tetrahydroisoquinolyl, tetrahydroquinolyl and the like.

“Heteroarylalkyl” or “heteroaralkyl” means an alkyl group substituted with a heteroaryl group in which the heteroaryl and alkyl are as previously described. Preferred heteroaralkyls contain a lower alkyl group. Non-limiting examples of suitable heteroaralkyl groups include pyridylmethyl, 2-(furan-3-yl)ethyl and quinolin-3-ylmethyl. The bond to the parent moiety is through the alkyl. “Heteroarylalkoxy” means a heteroaryl-alkyl-O— group in which the heteroaryl and alkyl are as previously described.

“Heterocyclyl” means a non-aromatic monocyclic or multicyclic ring system comprising about 3 to about 12 ring atoms, preferably about 5 to about 10 ring atoms, in which one or more of the atoms in the ring system is an element other than carbon, for example nitrogen, oxygen or sulfur, or combinations thereof. Preferred heterocyclyls contain about 5 to about 6 ring atoms. The prefix aza, oxa or thia before the heterocyclyl root name means that at least a nitrogen, oxygen or sulfur atom respectively is present as a ring atom. The heterocyclyl can be optionally substituted with one or more “ring system substituents” which may be the same or different, and are as defined herein. The nitrogen or sulfur atom of the heterocyclyl can be optionally oxidized to the corresponding N-oxide, S-oxide or S-dioxide. Non-limiting examples of suitable monocyclic heterocyclyl rings include piperidyl, pyrrolidinyl, piperazinyl, morpholinyl, thiomorpholinyl, thiazolidinyl, 1,3-dioxolanyl, 1,4-dioxanyl, tetrahydrofuranyl, tetrahydrothiophenyl, tetrahydrothiopyranyl, lactam, lactone, and the like. A heterocyclic ring may be fully saturated or may contain one or more units of unsaturation but is not aromatic.

“Heterocyclylalkyl” means an alkyl group substituted with a heterocyclyl group in which the heterocyclyl and alkyl groups are as previously described. Preferred heterocyclylalkyls contain a lower alkyl group. The bond to the parent moiety is through the alkyl.
“Ring system substituent” means a substituent attached to an aromatic or non-aromatic ring system that, for example, replaces an available hydrogen on the ring system. Ring system substituents may be the same or different, each being independently selected from the group consisting of aryl, heteroaryl, aralkyl, alkylaryl, aralkenyl, heteroaralkyl, alkylheteroaryl, heteroaralkenyl, hydroxy, hydroxyalkyl, alkoxy, aryloxy, aralkoxy, acyl, aroyl, halo, nitro, cyano, carboxy, alkoxycarbonyl, aryloxycarbonyl, aralkoxycarbonyl, alkylsulfonyl, arylsulfonyl, heteroarylsulfonyl, alkylsulfinyl, arylsulfinyl, heteroarylsulfinyl, alkylthio, arylthio, heteroarylthio, aralkylthio, heteroaralkylthio, cycloalkyl, cycloalkenyl, heterocyclyl, heterocyclenyl, Y₁Y₂N—, Y₁Y₂N-alkyl-, Y₁Y₂NC(O)— and Y₁Y₂NSO₂—, wherein Y₁ and Y₂ may be the same or different and are independently selected from the group consisting of hydrogen, alkyl, aryl, and aralkyl. “Ring system substituent” may also mean a single moiety which simultaneously replaces two available hydrogens on two adjacent carbon atoms (one H on each carbon) on a ring system. Examples of such moiety are methylene dioxy, ethylenedioxy, —C(CH₃)₂— and the like which form moieties such as, for example:

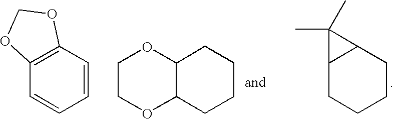

“Hydroxyalkyl” means a HO-alkyl- group in which alkyl is as previously defined. Preferred hydroxyalkyls contain lower alkyl. Non-limiting examples of suitable hydroxyalkyl groups include hydroxymethyl and 2-hydroxyethyl.

“Alkylamino” means an —NH₂ or —NH₃⁺ group in which one or more of the hydrogen atoms on the nitrogen is replaced by an alkyl group as defined above.

“Haloalkyl” means a halo-alkyl- group in which alkyl is as previously defined. Preferred haloalkyls contain lower alkyl.

“Alkoxyalkyl” means an alkoxy-alkyl group in which alkyl is as previously defined. Preferred alkoxyalkyls contain lower alkyl.

Also included in the scope of this invention are oxidized forms of the heteroatoms (e.g., nitrogen and sulfur) that are present in the compounds of this invention. Such oxidized forms include N(O)[N⁺—O⁻], S(O) and S(O)₂.

The term “isolated” or “in isolated form” for a compound refers to the physical state of said compound after being isolated from a synthetic process or natural source or combination thereof. The term “purified” or “in purified form” for a compound refers to the physical state of said compound after being obtained from a purification process or processes described herein or well known to the skilled artisan, in sufficient purity to be characterizable by standard analytical techniques described herein or well known to the skilled artisan.

When a functional group in a compound is termed “protected”, this means that the group is in modified form to preclude undesired side reactions at the protected site when the compound is subjected to a reaction. Suitable protecting groups will be recognized by those with ordinary skill in the art as well as by reference to standard textbooks such as, for example, T. W. Greene et al, Protective Groups in organic Synthesis (1991), Wiley, New York.

As used herein, the term “composition” is intended to encompass a product comprising the specified ingredients in the specified amounts, as well as any product which results, directly or indirectly, from combination of the specified ingredients in the specified amounts.

Isomers of the compounds of Formula I (where they exist), including enantiomers, stereoisomers, rotamers, tautomers and racemates are also contemplated as being part of this invention. The invention includes d and l isomers in both pure form and in admixture, including racemic mixtures. Isomers can be prepared using conventional techniques, either by reacting optically pure or optically enriched starting materials or by separating isomers of a compound of the Formula I. Isomers may also include geometric isomers, e.g., when a double bond is present. Polymorphous forms of the compounds of Formula I, whether crystalline or amorphous, also are contemplated as being part of this invention. The (+) isomers of the present compounds are preferred compounds of the present invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon are also within the scope of this invention.

It will be apparent to one skilled in the art that certain compounds of this invention may exist in alternative tautomeric forms. All such tautomeric forms of the present compounds are within the scope of the invention. Unless otherwise indicated, the representation of either tautomer is meant to include the other. For example, both isomers (1) and (2) are contemplated: 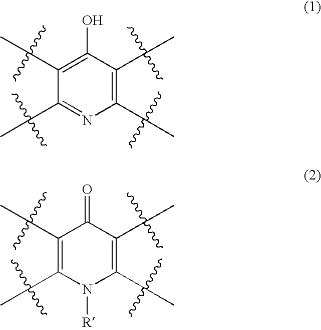
wherein R′ is H or C₁₋₆ unsubstituted alkyl.

Prodrugs and solvates of the compounds of the invention are also contemplated herein. The term “prodrug”, as employed herein, denotes a compound that is a drug precursor which, upon administration to a subject, undergoes chemical conversion by metabolic or chemical processes to yield a compound of formula I or a salt, ester and/or solvate thereof (e.g., a prodrug on being brought to the physiological pH or through enzyme action is converted to the desired drug form). A discussion of prodrugs is provided in T. Higuchi and V. Stella, Pro-drugs as Novel Delivery Systems (1987) Volume 14 of the A.C.S. Symposium Series, and in Bioreversible Carriers in Drug Design, (1987) Edward B. Roche, ed., American Pharmaceutical Association and Pergamon Press, both of which are incorporated herein by reference thereto.

“Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

One or more compounds of the invention may also exist as, or optionally converted to, a solvate. Preparation of solvates is generally known. Thus, for example, M. Caira et al, J. Pharmaceutical Sci., 93(3), 601-611 (2004) describe the preparation of the solvates of the antifungal fluconazole in ethyl acetate as well as from water. Similar preparations of solvates, hemisolvate, hydrates and the like are described by E. C. van Tonder et al, AAPS Pharm Sci Tech., 5(1), article 12 (2004); and A. L. Bingham et al, Chem. Commun., 603-604 (2001). A typical, non-limiting, process involves dissolving a compound in desired amounts of the desired solvent (organic or water or mixtures thereof) at a higher than ambient temperature, and cooling the solution at a rate sufficient to form crystals which are then isolated by standard methods. Analytical techniques such as, for example I.R. spectroscopy, show the presence of the solvent (or water) in the crystals as a solvate (or hydrate).

“Effective amount” or “therapeutically effective amount” is meant to describe an amount of a compound or a composition of the present invention effective in inhibiting mitotic kinesins, in particular KSP kinesin activity, and thus producing the desired therapeutic, ameliorative, inhibitory or preventative effect in a suitable subject.

The compounds of formula I form salts which are also within the scope of this invention. Reference to a compound of formula I herein is understood to include reference to salts, esters and solvates thereof, unless otherwise indicated. The term “salt(s)”, as employed herein, denotes acidic salts formed with inorganic and/or organic acids, as well as basic salts formed with inorganic and/or organic bases. In addition, when a compound of formula I contains both a basic moiety, such as, but not limited to a pyridine or imidazole, and an acidic moiety, such as, but not limited to a carboxylic acid, zwitterions (“inner salts”) may be formed and are included within the term “salt(s)” as used herein. Pharmaceutically acceptable (i.e., non-toxic, physiologically acceptable) salts are preferred, although other salts are also useful. Salts of the compounds of the formula I may be formed, for example, by reacting a compound of formula I with an amount of acid or base, such as an equivalent amount, in a medium such as one in which the salt precipitates or in an aqueous medium followed by lyophilization. Acids (and bases) which are generally considered suitable for the formation of pharmaceutically useful salts from basic (or acidic) pharmaceutical compounds are discussed, for example, by S. Berge et al, Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; in The Orange Book (Food & Drug Administration, Washington, D.C. on their website); and P. Heinrich Stahl, Camille G. Wermuth (Eds.), Handbook of Pharmaceutical Salts: Properties, Selection, and Use, (2002) Int'l. Union of Pure and Applied Chemistry, pp. 330-331. These disclosures are incorporated herein by reference thereto.

Exemplary acid addition salts include acetates, adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, methyl sulfates, 2-naphthalenesulfonates, nicotinates, nitrates, oxalates, pamoates, pectinates, persulfates, 3-phenylpropionates, phosphates, picrates, pivalates, propionates, salicylates, succinates, sulfates, sulfonates (such as those mentioned herein), tartarates, thiocyanates, toluenesulfonates (also known as tosylates) undecanoates, and the like.

Exemplary basic salts include ammonium salts, alkali metal salts such as sodium, lithium, and potassium salts, alkaline earth metal salts such as calcium and magnesium salts, aluminum salts, zinc salts, salts with organic bases (for example, organic amines) such as benzathines, diethylamine, dicyclohexylamines, hydrabamines (formed with N,N-bis(dehydroabietyl)ethylenediamine), N-methyl-D-glucamines, N-methyl-D-glucamides, t-butyl amines, piperazine, phenylcyclohexylamine, choline, tromethamine, and salts with amino acids such as arginine, lysine and the like. Basic nitrogen-containing groups may be quarternized with agents such as lower alkyl halides (e.g. methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

All such acid salts and base salts are intended to be pharmaceutically acceptable salts within the scope of the invention. All acid and base salts, as well as esters and solvates, are considered equivalent to the free forms of the corresponding compounds for purposes of the invention.

Pharmaceutically acceptable esters of the present compounds include the following groups: (1) carboxylic acid esters obtained by esterification of the hydroxy groups, in which the non-carbonyl moiety of the carboxylic acid portion of the ester grouping is selected from straight or branched chain alkyl (for example, acetyl, n-propyl, t-butyl, or n-butyl), alkoxyalkyl (for example, methoxymethyl), aralkyl (for example, benzyl), aryloxyalkyl (for example, phenoxymethyl), aryl (for example, phenyl optionally substituted with, for example, halogen, C₁₋₄alkyl, or C₁₋₄alkoxy or amino); (2) sulfonate esters, such as alkyl- or aralkylsulfonyl (for example, methanesulfonyl); (3) amino acid esters (for example, L-valyl or L-isoleucyl); (4) phosphonate esters and (5) mono-, di- or triphosphate esters. The phosphate esters may be further esterified by, for example, a C₁₋₂₀ alcohol or reactive derivative thereof, or by a 2,3-di(C₆₋₂₄)acyl glycerol.

In such esters, unless otherwise specified, any alkyl moiety present preferably contains from 1 to 18 carbon atoms, particularly from 1 to 6 carbon atoms, more particularly from 1 to 4 carbon atoms. Any cycloalkyl moiety present in such esters preferably contains from 3 to 6 carbon atoms. Any aryl moiety present in such esters preferably comprises a phenyl group.

Generally, the compounds of Formula I can be prepared by a variety of methods as disclosed in the examples hereinbelow.

One embodiment of the present invention relates to a process for preparing the compound of formula I comprises reacting a compound of formula II 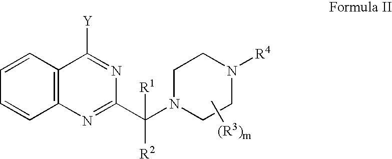
with R⁵OH or HN(R⁶)₂; wherein in formula II, Y is a halogen, and m, R¹, R², R³, and R⁴ are as set forth for formula I above; wherein reacting the compound of formula II with R⁵OH or HN(R⁶)₂ produces the compound of formula I wherein X is respectively R⁵OH or N(R⁶)₂.

Another embodiment of the present invention refers to the method of preparing the compound of formula I, wherein X is N(R⁶)₂ and wherein the compound of formula II is reacted with HN(R⁶)₂.

Another embodiment of the present invention refers to the method of preparing the compound of formula I, wherein X is N(R⁶)₂, wherein the compound of formula II is reacted with HN(R⁶)₂, and wherein R¹ and R² are both hydrogen.

Another embodiment of the present invention refers to the method of preparing the compound of formula I, wherein X is N(R⁶)₂, wherein the compound of formula II is reacted with HN(R⁶)₂, wherein R¹ and R² are both hydrogen, and wherein m is 0 or 1.

Another embodiment of the present invention refers to the method of preparing the compound of formula I, wherein X is OR⁵, and wherein the compound of formula II is reacted with R⁵OH.

Another embodiment of the present invention refers to the method of preparing the compound of formula I, wherein X is OR⁵, wherein the compound of formula II is reacted with R⁵OH, and wherein R¹ and R² are both hydrogen.

Another embodiment of the present invention refers to the method of preparing the compound of formula I, wherein X is OR⁵, wherein the compound of formula II is reacted with R⁵OH, wherein R¹ and R² are both hydrogen, and wherein wherein m is 0 or 1.

Another embodiment of the present invention relates to a process for preparing a compound of formula I comprising reacting a compound of formula III 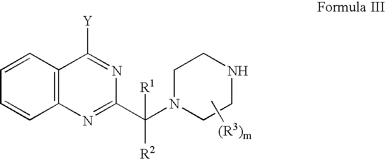
with R⁴Z, wherein Z is a halogen, R⁴ is as set forth in formula I, and m, X, R¹, R², and R³ in formula III are as set forth in formula I.

Another embodiment of the present invention refers to the method of preparing the compound of formula I, according to the above method involving formula III and R⁴Z, wherein R¹ and R² in formula I and III are both hydrogen.

Another embodiment of the present invention refers to the method of preparing the compound of formula I, according to the above method involving formula III and R⁴Z, wherein R¹ and R² in formula I and III are both hydrogen, and wherein m in formula I and III is 0 or 1.

Another embodiment of the present invention relates to a process for preparing a compound of formula I comprising reacting a compound of formula IV 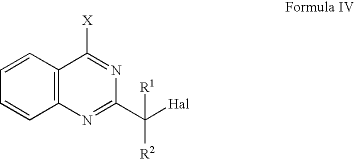
with a compound of formula V 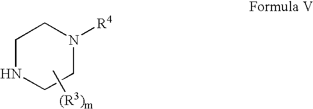
wherein Hal in formula IV is a halogen; X, R¹, and R² in formula IV and R³ and R⁴ in formula V are as set forth in formula I, provided that both R¹ and R² are not alkyl.

Another embodiment of the present invention refers to the method of preparing the compound of formula I, according to the above method involving formula IV and formula V, wherein both R¹ and R² in formula I and formula IV are hydrogen.

Another embodiment of the present invention refers to the method of preparing the compound of formula I, according to the above method involving formula IV and formula V, wherein both R¹ and R² in formula I and formula IV are hydrogen; and wherein m in formula I and formula V is 0 or 1.

Another embodiment of the present invention relates to a process for preparing a compound of formula I wherein R⁴ in formula I is —(C═O)—NHR⁹, comprising reacting a compound of formula III 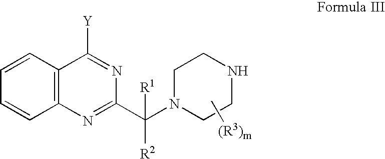
with a compound of formula R⁹—N═C═O; wherein R⁹, and m, X, R¹, R², and R³ are as set forth in formula I.

The compounds of the invention can be used to treat cellular proliferation diseases. Such disease states which can be treated by the compounds, compositions and methods provided herein include, but are not limited to, cancer (further discussed below), hyperplasia, cardiac hypertrophy, autoimmune diseases, fungal disorders, arthritis, graft rejection, inflammatory bowel disease, immune disorders, inflammation, cellular proliferation induced after medical procedures, including, but not limited to, surgery, angioplasty, and the like. Treatment includes inhibiting cellular proliferation. It is appreciated that in some cases the cells may not be in a hyper- or hypoproliferation state (abnormal state) and still require treatment. For example, during wound healing, the cells may be proliferating “normally”, but proliferation enhancement may be desired. Thus, in one embodiment, the invention herein includes application to cells or subjects afflicted or subject to impending affliction with any one of these disorders or states.

The compounds, compositions and methods provided herein are particularly useful for the treatment of cancer including solid tumors such as skin, breast, brain, colon, gall bladder, thyroid, cervical carcinomas, testicular carcinomas, etc. More particularly, cancers that may be treated by the compounds, compositions and methods of the invention include, but are not limited to:

Cardiac: sarcoma (angiosarcoma, fibrosarcoma, rhabdomyosarcoma, liposarcoma), myxoma, rhabdomyoma, fibroma, lipoma and teratoma;

Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma;

Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma);

Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma);

Liver: hepatoma (hepatocellular carcinoma), cholangiocarcinoma, hepatoblastoma, angiosarcoma, hepatocellular adenoma, hemangioma;

Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors;

Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma);

Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma);

Hematologic: blood (myeloid leukemia (acute and chronic), acute lymphoblastic leukemia, acute and chronic lymphocytic leukemia, myeloproliferative diseases, multiple myeloma, myelodysplastic syndrome), Hodgkin's disease, non-Hodgkin's lymphoma (malignant lymphoma), B-cell lymphoma, T-cell lymphoma, hairy cell lymphoma, Burkeft's lymphoma, promyelocytic leukemia;

Skin: malignant melanoma, basal cell carcinoma, squamous cell carcinoma, Karposi's sarcoma, moles dysplastic nevi, lipoma, angioma, dermatofibroma, keloids, psoriasis;

Adrenal glands: neuroblastoma; and

Other tumors: including xenoderoma pigmentosum, keratoctanthoma and thyroid follicular cancer.

As used herein, treatment of cancer includes treatment of cancerous cells, including cells afflicted by any one of the above-identified conditions.

The compounds of the present invention may also be useful in the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse.

The compounds of the present invention may also be useful in inhibiting tumor angiogenesis and metastasis.

The compounds of the present invention may also be useful as antifungal agents, by modulating the activity of the fungal members of the bimC kinesin subgroup, as is described in U.S. Pat. No. 6,284,480.

The present compounds are also useful in combination with one or more other known therapeutic agents and anti-cancer agents. Combinations of the present compounds with other anti-cancer or chemotherapeutic agents are within the scope of the invention. Examples of such agents can be found in Cancer Principles and Practice of Oncology by V. T. Devita and S. Hellman (editors), 6^{th} edition (Feb. 15, 2001), Lippincoft Williams & Wilkins Publishers. A person of ordinary skill in the art would be able to discern which combinations of agents would be useful based on the particular characteristics of the drugs and the cancer involved. Such anti-cancer agents include, but are not limited to, the following: estrogen receptor modulators, androgen receptor modulators, retinoid receptor modulators, cytotoxic/cytostatic agents, antiproliferative agents, prenyl-protein transferase inhibitors, HMG-CoA reductase inhibitors and other angiogenesis inhibitors, inhibitors of cell proliferation and survival signaling, apoptosis inducing agents and agents that interfere with cell cycle checkpoints. The present compounds are also useful when co-administered with radiation therapy.

The phrase “estrogen receptor modulators” refers to compounds that interfere with or inhibit the binding of estrogen to the receptor, regardless of mechanism. Examples of estrogen receptor modulators include, but are not limited to, tamoxifen, raloxifene, idoxifene, LY353381, LY117081, toremifene, fulvestrant, 4-[7-(2,2-dimethyl-1-oxopropoxy-4-methyl-2-[4-[2-(1-piperidinyl)ethoxy]phenyl]-2H-1-benzopyran-3-yl]-phenyl-2,2-dimethylpropanoate, 4,4′-dihydroxybenzophenone-2,4-dinitrophenyl-yd razone, aid SH646.

The phrase “androgen receptor modulators” refers to compounds which interfere or inhibit the binding of androgens to the receptor, regardless of mechanism. Examples of androgen receptor modulators include finasteride and other 5α-reductase inhibitors, nilutamide, flutamide, bicalutamide, liarozole, and abiraterone acetate.

The phrase “retinoid receptor modulators” refers to compounds which interfere or inhibit the binding of retinoids to the receptor, regardless of mechanism. Examples of such retinoid receptor modulators include bexarotene, tretinoin, 13-cis-retinoic acid, 9-cis-retinoic acid, a difluoromethylornithine, ILX23-7553, trans-N-(4′-hydroxyphenyl)retinamide, and N-4-carboxyphenyl retinamide.

The phrase “cytotoxic/cytostatic agents” refer to compounds which cause cell death or inhibit cell proliferation primarily by interfering directly with the cell's functioning or inhibit or interfere with cell mycosis, including alkylating agents, tumor necrosis factors, intercalators, hypoxia activatable compounds, microtubule inhibitors/microtubule-stabilizing agents, inhibitors of mitotic kinesins, inhibitors of kinases involved in mitotic progression, antimetabolites; biological response modifiers; hormonal/anti-hormonal therapeutic agents, haematopoietic growth factors, monoclonal antibody targeted therapeutic agents, monoclonal antibody therapeutics, topoisomerase inhibitors, proteasome inhibitors and ubiquitin ligase inhibitors.

Examples of cytotoxic agents include, but are not limited to, sertenef, cachectin, ifosfamide, tasonermin, lonidamine, carboplatin, altretamine, prednimustine, dibromodulcitol, ranimustine, fotemustine, nedaplatin, oxaliplatin, temozolomide (TEMODAR™ from Schering-Plough Corporation, Kenilworth, N.J.), cyclophosphamide, heptaplatin, estramustine, improsulfan tosilate, trofosfamide, nimustine, dibrospidium chloride, pumitepa, lobaplatin, satraplatin, profiromycin, cisplatin, doxorubicin, irofulven, dexifosfamide, cis-aminedichloro(2-methyl-pyridine)platinum, benzylguanine, glufosfamide, GPX100, (trans, trans, trans)-bis-mu-(hexane-1,6-diamine)-mu-[diamine-platinum(II)]bis[diamine(chloro)platinum(II)] tetrachloride, diarizidinylspermine, arsenic trioxide, 1-(11-dodecylamino-10-hydroxyundecyl)-3,7-dimethylxanthine, zorubicin, idarubicin, daunorubicin, bisantrene, mitoxantrone, pirarubicin, pinafide, valrubicin, amrubicin, antineoplaston, 3′-deansino-3′-morpholino-13-deoxo-10-hydroxycarminomycin, annamycin, galarubicin, elinafide, MEN10755, 4-demethoxy-3-deamino-3-aziridinyl-4-methylsulphonyl-daunombicin (see WO 00/50032), methoxtrexate, gemcitabine, and mixture thereof.

An example of a hypoxia activatable compound is tirapazamine.

Examples of proteasome inhibitors include, but are not limited to, lactacystin and bortezomib.

Examples of microtubule inhibitors/microtubule-stabilising agents include paclitaxel, vindesine sulfate, 3′,4′-didehydro-4′-deoxy-8′-norvincaleukoblastine, docetaxel, rhizoxin, dolastatin, mivobulin isethionate, auristatin, cemadotin, RPR109881, BMS184476, vinflunine, cryptophycin, 2,3,4,5,6-pentafluoro-N-(3-fluoro-4-methoxyphenyl)benzene sulfonamide, anhydrovinblastine, N,N-dimethyl-L-valyl-L-valyl-N-methyl-L-valyl-L-prolyl-L-proline-t-butylamide, TDX258, the epothilones (see for example U.S. Pat. Nos. 6,284,781 and 6,288,237) and BMS188797.

Some examples of topoisomerase inhibitors are topotecan, hycaptamine, irinotecan, rubitecan, 6-ethoxypropionyl-3′,4′-O-exo-benzylidene-chartreusin, 9-methoxy-N,N-dimethyl-5-nitropyrazolo[3,4,5-kl]acridine-2-(6H) propanamine, 1-amino-9-ethyl-5-fluoro-2,3-dihydro-9-hydroxy-4-methyl-1H, 12H-benzo[de]pyrano[3′,4′:b,7]-indolizino[1,2b]quinoline-10,13(9H,15H)dione, lurtotecan, 7-[2-(N-isopropylamino)ethyl]-(20S)camptothecin, BNP1350, BNPI1100, BN80915, BN80942, etoposide phosphate, teniposide, sobuzoxane, 2′-dimethylamino-2′-deoxy-etoposide, GL331, N-[2-(dimethylamino)ethyl]-9-hydroxy-5,6-dimethyl-6H-pyrido[4,3-b]carbazole-1-carboxamide, asulacrine, (5a,5aB,8aa,9b)-9-[2-[N-[2-(dimethylamino)ethyl]-N-methylamino]ethyl]-5-[4-hydroxy-3,5-dimethoxyphenyl]-5,5a,6,8,8a,9-hexohydrofuro(3′,4′:6,7)naphtho(2,3-d)-1,3-dioxol-6-one, 2,3-(methylenedioxy)-5-methyl-7-hydroxy-8-methoxybenzo[c]-phenanthridinium, 6,9-bis[(2-aminoethyl)amino]benzo[g]isoguinoline-5,10-dione, 5-(3-aminopropylamino)-7,10-dihydroxy-2-(2-hydroxyethylaminomethyl)-6H-pyrazolo[4,5,1-de]acridin-6-one, N-[1-[2-(diethylamino)ethylamino]-7-methoxy-9-oxo-9H-thioxanthen-4-ylmethyl]formamide, N-(2-(dimethylamino)ethyl)acridine-4-carboxamide, 6-[[2-(dimethylamino)ethyl]amino]-3-hydroxy-7H-indeno[2,1-c]quinolin-7-one, dimesna, and camptostar.

Other useful anti-cancer agents that can be used in combination with the present compounds include thymidilate synthase inhibitors, such as 5-fluorouracil.

In one embodiment, inhibitors of mitotic kinesins include, but are not limited to, inhibitors of KSP, inhibitors of MKLP1, inhibitors of CENP-E, inhibitors of MCAK, inhibitors of Kifl4, inhibitors of Mphosph1 and inhibitors of Rab6-KIFL.

The phrase “inhibitors of kinases involved in mitotic progression” include, but are not limited to, inhibitors of aurora kinase, inhibitors of Polo-like kinases (PLK) (in particular inhibitors of PLK-1), inhibitors of bub-1 and inhibitors of bub-R1.

The phrase “antiproliferative agents” includes antisense RNA and DNA oligonucleotides such as G3139, ODN698, RVASKRAS, GEM231, and INX3001, and antimetabolites such as enocitabine, carmofur, tegafur, pentostatin, doxifluridine, trimetrexate, fludarabine, capecitabine, galocitabine, cytarabine ocfosfate, fosteabine sodium hydrate, raltitrexed, paltitrexid, emitefur, tiazofurin, decitabine, nolatrexed, pemetrexed, nelzarabine, 2′-deoxy-2′-methylidenecytidine, 2′-fluoromethylene-2′-deoxycytidine, N-[5-(2,3-dihydro-benzofuryl)sulfonyl]-N′-(3,4-dichlorophenyl)urea, N6-[4-deoxy-4-[N2-[2(E),4(E)-tetradecadienoyl]glycylamino]-L-glycero-B-L-manno-heptopyranosyl]adenine, aplidine, ecteinascidin, troxacitabine, 4-[2-amino-4-oxo-4,6,7,8-tetrahydro-3H-pyrimidino[5,4-b][1,4]thiazin-6-yl-(S)-ethyl]-2,5-thienoyl-L-glutamic acid, aminopterin, 5-flurouracil, alanosine, 11-acetyl-8-(carbamoyloxymethyl)-4-formyl-6-methoxy-14-oxa-1,11-diazatetracyclo(7.4.1.0.0)-tetradeca-2,4,6-trien-9-yl acetic acid ester, swainsonine, lometrexol, dexrazoxane, methioninase, 2′-cyano-2′-deoxy-N-4-palmitoyl-1-B-D-arabino furanosyl cytosine and 3-aminopyridine-2-carboxaldehyde thiosemicarbazone.

Examples of monoclonal antibody targeted therapeutic agents include those therapeutic agents which have cytotoxic agents or radioisotopes attached to a cancer cell specific or target cell specific monoclonal antibody. Examples include Bexxar.

Examples of monoclonal antibody therapeutics useful for treating cancer include Erbitux (Cetuximab).

The phrase “HMG-CoA reductase inhibitors” refers to inhibitors of 3-hydroxy-3-methylglutaryl-CoA reductase. Examples of HMG-CoA reductase inhibitors that may be used include but are not limited to lovastatin (MEVACOR®; see U.S. Pat. Nos. 4,231,938, 4,294,926 and 4,319,039), simvastatin (ZOCOR®; see U.S. Pat. Nos. 4,444,784, 4,820,850 and 4,916,239), pravastatin (PRAVACHOL®; see U.S. Pat. Nos. 4,346,227, 4,537,859, 4,410,629, 5,030,447 and 5,180,589), fluvastatin (LESCOL®; see U.S. Pat. Nos. 5,354,772, 4,911,165, 4,929,437, 5,189,164, 5,118,853, 5,290,946 and 5,356,896) and atorvastatin (LIPITOR®; see U.S. Pat. Nos. 5,273,995, 4,681,893, 5,489,691 and 5,342,952). The structural formulas of these and additional HMG-CoA reductase inhibitors that may be used in the instant methods are described at page 87 of M. Yalpani, “Cholesterol Lowering Drugs”, Chemistry & Industry, pp. 85-89 (5 Feb. 1996) and U.S. Pat. Nos. 4,782,084 and 4,885,314. The term HMG-CoA reductase inhibitor as used herein includes all pharmaceutically acceptable lactone and open-acid forms (i.e., where the lactone ring is opened to form the free acid) as well as salt and ester forms of compounds which have HMG-CoA reductase inhibitory activity, and therefore the use of such salts, esters, open acid and lactone forms is included in the scope of this invention.

The phrase “prenyl-protein transferase inhibitor” refers to a compound which inhibits any one or any combination of the prenyl-protein transferase enzymes, including farnesyl-protein transferase (FPTase), geranylgeranyl-protein transferase type I (GGPTase-I), and geranylgeranyl-protein transferase type-II (GGPTase-II, also called Rab GGPTase).

Examples of prenyl-protein transferase inhibitors can be found in the following publications and patents: WO 96/30343, WO 97/18813, WO 97/21701, WO 97/23478, WO 97/38665, WO 98/28980, WO 98/29119, WO 95/32987, U.S. Pat. Nos. 5,420,245, 5,523,430, 5,532,359, 5,510,510, 5,589,485, 5,602,098, European Patent Publ. 0 618 221, European Patent Publ. 0 675 112, European Patent Publ. 0 604181, European Patent Publ. 0 696 593, WO 94/19357, WO 95/08542, WO 95/11917, WO 95/12612, WO 95/12572, WO 95/10514, U.S. Pat. No. 5,661,152, WO 95/10515, WO 95/10516, WO 95/24612, WO 95/34535, WO 95/25086, WO 96/05529, WO 96/06138, WO 96/06193, WO 96/16443, WO 96/21701, WO 96/21456, WO 96/22278, WO 96/24611, WO 96/24612, WO 96/05168, WO 96/05169, WO 96/00736, U.S. Pat. No. 5,571,792, WO 96/17861, WO 96/33159, WO 96/34850, WO 96/34851, WO 96/30017, WO 96/30018, WO 96/30362, WO 96/30363, WO 96/31111, WO 96/31477, WO 96/31478, WO 96/31501, WO 97/00252, WO 97/03047, WO 97/03050, WO 97/04785, WO 97/02920, WO 97/17070, WO 97/23478, WO 97/26246, WO, 97/30053, WO 97/44350, WO 98/02436, and U.S. Pat. No. 5,532,359. For an example of the role of a prenyl-protein transferase inhibitor on angiogenesis see European of Cancer, Vol. 35, No. 9, pp. 1394-1401(1999).

Examples of farnesyl protein transferase inhibitors include SARASAR™ (4-[2-[4-[(11R)-3,10-dibromo-8-chloro-6,11-dihydro-5H-benzo[5,6]cyclohepta[1,2-b]pyridin-11-yl-]-1-piperidinyl]-2-oxoehtyl]-1-piperidinecarboxamide from Schering-Plough Corporation, Kenilworth, N.J.), tipifarnib (Zarnestra® or R115777 from Janssen Pharmaceuticals), L778,123 (a farnesyl protein transferase inhibitor from Merck & Company, Whitehouse Station, N.J.), BMS 214662 (a farnesyl protein transferase inhibitor from Bristol-Myers Squibb Pharmaceuticals, Princeton, N.J.).

The phrase “angiogenesis inhibitors” refers to compounds that inhibit the formation of new blood vessels, regardless of mechanism. Examples of angiogenesis inhibitors include, but are not limited to, tyrosine kinase inhibitors, such as inhibitors of the tyrosine kinase receptors Flt-1 (VEGFR¹) and Flk-1/KDR (VEGFR2), inhibitors of epidermal-derived, fibroblast-derived, or platelet derived growth factors, MMP (matrix metalloprotease) inhibitors, integrin blockers, interferon-α (for example Intron and Peg-Intron), interleukin-12, pentosan polysulfate, cyclooxygenase inhibitors, including nonsteroidal anti-inflammatories (NSAIDs) like aspirin and ibuprofen as well as selective cyclooxygenase-2 inhibitors like celecoxib and rofecoxib (PNAS, Vol. 89, p. 7384 (1992); JNCI, Vol. 69, p. 475 (1982); Arch. Opthalmol., Vol. 108, p. 573 (1990); Anat. Rec., Vol. 238, p. 68 (1994); FEBS Letters, Vol. 372, p. 83 (1995); Clin. Orthop. Vol. 313, p. 76 (1995); J. Mol. Endocrinol., Vol. 16, p. 107 (1996); Jpn. J. Pharmacol., Vol. 75, p. 105 (1997); Cancer Res., Vol. 57, p. 1625 (1997); Cell, Vol. 93, p. 705 (1998); Intl. J. Mol. Med., Vol. 2, p. 715 (1998); J. Biol. Chem., Vol. 274, p. 9116 (1999)), steroidal anti-inflammatories (such as corticosteroids, mineralocorticoids, dexamethasone, prednisone, prednisolone, methylpred, betamethasone), carboxyamidotriazole, combretastatin A-4, squalamine, 6-O-chloroacetyl-carbonyl)-fumagillol, thalidomide, angiostatin, troponin-1, angiotensin II antagonists (see Fernandez et al., J. Lab. Clin. Med. 105:141-145 (1985)), and antibodies to VEGF (see, Nature Biotechnology, Vol. 17, pp. 963-968 (October 1999); Kim et al., Nature, 362, 841-844 (1993); WO 00/44777; and WO 00/61186).

Other therapeutic agents that modulate or inhibit angiogenesis and may also be used in combination with the compounds of the instant invention include agents that modulate or inhibit the coagulation and fibrinolysis systems (see review in Clin. Chem. La. Med. 38:679-692 (2000)). Examples of such agents that modulate or inhibit the coagulation and fibrinolysis pathways include, but are not limited to, heparin (see Thromb. Haemost. 80:10-23 (1998)), low molecular weight heparins and carboxypeptidase U inhibitors (also known as inhibitors of active thrombin activatable fibrinolysis inhibitor [TAFla]) (see Thrombosis Res. 101:329-354 (2001)). Examples of TAFla inhibitors have been described in PCT Publication WO 03/013,526.

The phrase “agents that interfere with cell cycle checkpoints” refers to compounds that inhibit protein kinases that transduce cell cycle checkpoint signals, thereby sensitizing the cancer cell to DNA damaging agents. Such agents include inhibitors of ATR, ATM, the Chk1 and Chk2 kinases and cdk and cdc kinase inhibitors and are specifically exemplified by 7-hydroxystaurosporin, flavopiridol, CYC202 (Cyclacel) and BMS-387032.

The phrase “inhibitors of cell proliferation and survival signaling pathway” refers to agents that inhibit cell surface receptors and signal transduction cascades downstream of those surface receptors. Such agents include inhibitors of EGFR (for example gefitinib and erlotinib), antibodies to EGFR (for example C225), inhibitors of ERB-2 (for example trastuzumab), inhibitors of IGFR, inhibitors of cytokine receptors, inhibitors of MET, inhibitors of PI3K (for example LY294002), serine/threonine kinases (including but not limited to inhibitors of Akt such as described in WO 02/083064, WO 02/083139, WO 02/083140 and WO 02/083138), inhibitors of Raf kinase (for example BAY-43-9006), inhibitors of MEEK (for example CI-1040 and PD-098059), inhibitors of mTOR (for example Wyeth CCI-779), and inhibitors of C-abl kinase (for example GLEEVEC™, Novartis Pharmaceuticals). Such agents include small molecule inhibitor compounds and antibody antagonists.

The phrase “apoptosis inducing agents” includes activators of TNF receptor family members (including the TRAIL receptors).

In one embodiment, the present invention provides a pharmaceutical composition comprising a therapeutically effective amount of a combination of at least one comound of formula I or a pharmaceutically acceptable salt, solvate or ester thereof and temozolomide.

In another embodiment, the present invention provides a method of treating a proliferative disease in a subject comprising administering to said subject in need of such treatment a therapeutically effective amount of a combination of of at least one comound of formula I or a pharmaceutically acceptable salt, solvate or ester thereof and temozolomide.

In another embodiment, the present invention provides a process for potentiating the growth activity suppression activity of temolozamide in cancer cells comprising administering to said cells therapeutically effective amount of a combination of at least one compound of formula I or a pharmaceutically acceptable salt, solvate or ester thereof and temozolomide.

In another embodiment, the cancer cells useful in the above process for potentiating the growth activity suppression activity of temolozolamide is selected from the group consisting of pancreatic and glioma cells.

The invention also encompasses combinations with NSAID's which are selective COX-2 inhibitors. For purposes of this specification NSAID's which are selective inhibitors of COX-2 are defined as those which possess a specificity for inhibiting COX-2 over COX-1 of at least 100 fold as measured by the ratio of IC50 for COX-2 over IC50 for COX-1 evaluated by cell or microsomal assays. Inhibitors of COX-2 that are particularly useful in the instant method of treatment are: 3-phenyl-4-(4-(methylsulfonyl)phenyl)-2-(5H)-furanone; and 5-chloro-3-(4-methylsulfonyl)phenyl-2-(2-methyl-5 pyridinyl)pyridine; or a pharmaceutically acceptable salt thereof.

Compounds that have been described as specific inhibitors of COX-2 and are therefore useful in the present invention include, but are not limited to, parecoxib, CELIEBREX® and BEXTRA® or a pharmaceutically acceptable salt thereof.

Other examples of angiogenesis inhibitors include, but are not limited to, endostatin, ukrain, ranpirnase, IM862, 5-methoxy-4-[2-methyl-3-(3-methyl-2-butenyl)oxiranyl]-1-oxaspiro[2,5]oct-6-yl(chloroacetyl)carbamate, acetyldinanaline, 5-amino-1-[[3,5-dichloro-4-(4-chlorobenzoyl)phenyl]methyl]-1H-1,2,3-triazole-4-carboxamide, CM101, squalamine, combretastatin, RP14610, NX31838, sulfated mannopentaose phosphate, 7,7-(carbonyl-bis[imino-N-methyl-4,2-pyrrolocarbonylimino[N-methyl-4,2-pyrrole]-carbonylimino]-bis-(1,3-naphthalene disulfonate), and 3-[(2,4-dimethylpyrrol-5-yl)methylene]-2-indolinone (SU5416).

As used above, “integrin blockers” refers to compounds which selectively antagonize, inhibit or counteract binding of a physiological ligand to the αᵥβ₃ integrin, to compounds which selectively antagonize, inhibit or counteract binding of a physiological ligand to the αᵥβ₅ integrin, to compounds which antagonize, inhibit or counteract binding of a physiological ligand to both the αᵥβ₃ integrin and the αᵥβ₅ integrin, and to compounds which antagonize, inhibit or counteract the activity of the particular integrin(s) expressed on capillary endothelial cells. The term also refers to antagonists of the αᵥβ₆, αᵥβ₈, α₁β₁, α₂β₁, α₅β₁, α₆β₁ and α₆β₄ integrins. The term also refers to antagonists of any combination of αᵥβ₃, αᵥβ₅, αᵥβ₆, αᵥβ₈, α₁β₁, α₂β₁, α₅β₁, α₆β₁, and α₆β₄ integrins.

Some examples of tyrosine kinase inhibitors include N-(trifluoromethylphenyl)-5-methyl isoxazol-4-carboxamide, 3-[(2,4-dimethylpyrrol-5-yl)methylidenyl)indolin-2-one, 17-(allylamino)-17-demethoxygeldanamycin, 4-(3-chloro-4-fluorophenylamino)-7-methoxy-6-[3-(4-morpholinyl)propoxyl]quinazoline, N-(3-ethynylphenyl)-6,7-bis(2-methoxyethoxy)-4-quinazolinamine, BIBX1382, 2,3,9,10,11,12-hexahydro-10-(hydroxymethyl)-10-hydroxy-9-methyl-9,12-epoxy-1H-diindolo[1,2,3-fg:3′,2′,1′-kl]pyrrolo[3,4-i][1,6]benzodiazocin-1-one, SH268, genistein, STI571, CEP2563, 4-(3-chlorophenylamino)-5,6-dimethyl-7H-pyrrolo[2,3-d]pyrimidinemethane sulfonate, 4-(3-bromo-4-hydroxyphenyl)amino-6,7-dimethoxyquinazoline, 4-(4′-hydroxyphenyl)amino-6,7-dimethoxyquinazoline, SU6668, STI571A, N -4-chlorophenyl-4-(4-pyridylmethyl)-1-phthalazinamine, and EMD121974.

Combinations with compounds other than anti-cancer compounds are also encompassed in the instant methods. For example, combinations of the present compounds with PPAR-γ (i.e., PPAR-gamma) agonists and PPAR-δ (i.e., PPAR-delta) agonists are useful in the treatment of certain malingnancies. PPAR-γ and PPAR-δ are the nuclear peroxisome proliferator-activated receptors γ and δ. The expression of PPAR-γ on endothelial cells and its involvement in angiogenesis has been reported in the literature (see J. Cardiovasc. Pharmacol. 1998; 31:909-913; J. Biol. Chem. 1999; 274:9116-9121; Invest. Ophthalmol Vis. Sci. 2000; 41:2309-2317). More recently, PPAR-γ agonists have been shown to inhibit the angiogenic response to VEGF in vitro; both troglitazone and rosiglitazone maleate inhibit the development of retinal neovascularization in mice (Arch. Ophthamol. 2001; 119:709-717). Examples of PPAR-γ agonists and PPAR-γ/α agonists include, but are not limited to, thiazolidinediones (such as DRF2725, CS-011, troglitazone, rosiglitazone, and pioglitazone), fenofibrate, gemfibrozil, clofibrate, GW2570, SB219994, AR-H039242, JTT-501, MCC-555, GW2331, GW409544, NN2344, KRP297, NP0110, DRF4158, NN622, GI262570, PNU182716, DRF552926, 2-[(5,7-dipropyl-3-trifluoromethyl-1,2-benzisoxazol-6-yl)oxy]-2-methylpropionic acid, and 2(R)-7-(3-(2-chloro-4-(4-fluorophenoxy)phenoxy)propoxy)-2-ethylchromane-2-carboxylic acid.

In one embodiment, useful anti-cancer (also known as anti-neoplastic) agents that can be used in combination with the present compounds include, but are not limited, to Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin (ELOXATIN™ from Sanofi-Synthelabo Pharmaeuticals, France), Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin (adriamycin), cyclophosphamide (cytoxan), gemcitabine, interferons, pegylated interferons, Erbitux and mixtures thereof.

Another embodiment of the present invention is the use of the present compounds in combination with gene therapy for the treatment of cancer. For an overview of genetic strategies to treating cancer, see Hall et al (Am J Hum Genet 61:785-789, 1997) and Kufe et al (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J Immunol 2000; 164:217-222).

The present compounds can also be administered in combination with one or more inhibitor of inherent multidrug resistance (MDR), in particular MDR associated with high levels of expression of transporter proteins. Such MDR inhibitors include inhibitors of p-glycoprotein (P-gp), such as LY335979, XR9576, OC144-093, R101922, VX853 and PSC833 (valspodar).

The present compounds can also be employed in conjunction with one or more anti-emetic agents to treat nausea or emesis, including acute, delayed, late-phase, and anticipatory emesis, which may result from the use of a compound of the present invention, alone or with radiation therapy. For the prevention or treatment of emesis, a compound of the present invention may be used in conjunction with one or more other anti-emetic agents, especially neurokinin-1 receptor antagonists, 5HT3 receptor, antagonists, such as ondansetron, granisetron, tropisetron, and zatisetron, GABAB receptor agonists, such as baclofen, a corticosteroid such as Decadron (dexamethasone), Kenalog, Aristocort, Nasalide, Preferid, Benecorten or those as described in U.S. Pat. Nos. 2,789,118, 2,990,401, 3,048,581, 3,126,375, 3,929,768, 3,996,359, 3,928,326 and 3,749,712, an antidopaminergic, such as the phenothiazines (for example prochlorperazine, fluphenazine, thioridazine and mesoridazine), metoclopramide or dronabinol. In one embodiment, an anti-emesis agent selected from a neurokinin-1 receptor antagonist, a 5HT3 receptor antagonist and a corticosteroid is administered as an adjuvant for the treatment or prevention of emesis that may result upon administration of the present compounds.

Examples of neurokinin-1 receptor antagonists that can be used in conjunction with the present compounds are described in U.S. Pat. Nos. 5,162,339, 5,232,929, 5,242,930, 5,373,003, 5,387,595, 5,459,270, 5,494,926, 5,496,833, 5,637,699, and 5,719,147, content of which are incorporated herein by reference. In an embodiment, the neurokinin-1 receptor antagonist for use in conjunction with the compounds of the present invention is selected from: 2-(R)-(1-(R)-(3,5-bis(trifluoromethyl)phenyl)ethoxy)-3-(S)-(4-fluorophenyl)-4-(3-(5-oxo-1H,4H-1,2,4-triazolo)methyl)morpholine, or a pharmaceutically acceptable salt thereof, which is described in U.S. Pat. No. 5,719,147.

A compound of the present invention may also be administered with one or more immunologic-enhancing drug, such as for example, levamisole, isoprinosine and Zadaxin.

Thus, the present invention encompasses the use of the present compounds (for example, for treating or preventing cellular proliferative diseases) in combination with a second compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drug, an inhibitor of cell proliferation and survival signaling, an agent that interfers with a cell cycle checkpoint, and an apoptosis inducing agent.

In one embodiment, the present invention empassesses the composition and use of the present compounds in combination with a second compound selected from: a cytostatic agent, a cytotoxic agent, taxanes, a topoisomerase II inhibitor, a topoisomerase I inhibitor, a tubulin interacting agent, hormonal agent, a thymidilate synthase inhibitors, anti-metabolites, an alkylating agent, a farnesyl protein transferase inhibitor, a signal transduction inhibitor, an EGFR kinase inhibitor, an antibody to EGFR, a C-abl kinase inhibitor, hormonal therapy combinations, and aromatase combinations.

The term “treating cancer” or “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

In one embodiment, the angiogenesis inhibitor to be used as the second compound is selected from a tyrosine kinase inhibitor, an inhibitor of epidermal-derived growth factor, an inhibitor of fibroblast-derived growth factor, an inhibitor of platelet derived growth factor, an MW (matrix metalloprotease) inhibitor, an integrin blocker, interferon-α, interleukin-12, pentosan polysulfate, a cyclooxygenase inhibitor, carboxyamidotriazole, combretastatin A-4, squalamine, 6-(O-chloroacetylcarbonyl)-fumagillol, thalidomide, angiostatin, troponin-1, or an antibody to VEGF. In an embodiment, the estrogen receptor modulator is tamoxifen or raloxifene.

Also included in the present invention is a method of treating cancer comprising administering a therapeutically effective amount of at least one compound of Formula I in combination with radiation therapy and at least one compound selected from: an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of inherent multidrug resistance, an anti-emetic agent, an immunologic-enhancing drag, an inhibitor of cell proliferation and survival signaling, an agent that interfers with a cell cycle checkpoint, and an apoptosis inducing agent.

Yet another embodiment of the invention is a method of treating cancer comprising administering a therapeutically effective amount of at least one compound of Formula I in combination with paclitaxel or trastuzumab.

The present invention also includes a pharmaceutical composition useful for treating or preventing cellular proliferation diseases (such as cancer, hyperplasia, cardiac hypertrophy, autoimmune diseases, fungal disorders, arthritis, graft rejection, inflammatory bowel disease, immune disorders, inflammation, and cellular proliferation induced after medical procedures) that comprises a therapeutically effective amount of at least one compound of Formula I and at least one compound selected from: an estrogen receptor modulator, an androgen receptor modulator, a retinoid receptor modulator, a cytotoxic/cytostatic agent, an antiproliferative agent, a prenyl-protein transferase inhibitor, an HMG-CoA reductase inhibitor, an angiogenesis inhibitor, a PPAR-γ agonist, a PPAR-δ agonist, an inhibitor of cell proliferation and survival signaling, an agent that interfers with a cell cycle checkpoint, and an apoptosis inducing agent.

A preferred dosage is about 0.001 to 500 mg/kg of body weight/day of a compound of Formula I or a pharmaceutically acceptable salt or ester thereof. An especially preferred dosage is about 0.01 to 25 mg/kg of body weight/day of a compound of Formula I or a pharmaceutically acceptable salt or ester thereof.

The phrases “effective amount” and “therapeutically effective amount” mean that amount of a compound of Formula I, and other pharmacological or therapeutic agents described herein, that will elicit a biological or medical response of a tissue, a system, or a subject (e.g., animal or human) that is being sought by the administrator (such as a researcher, doctor or veterinarian) which includes alleviation of the symptoms of the condition or disease being treated and the prevention, slowing or halting of progression of one or more cellular proliferation diseases. The formulations or compositions, combinations and treatments of the present invention can be administered by any suitable means which produce contact of these compounds with the site of action in the body of, for example, a mammal or human.

For administration of pharmaceutically acceptable salts of the above compounds, the weights indicated above refer to the weight of the acid equivalent or the base equivalent of the therapeutic compound derived from the salt.

As described above, this invention includes combinations comprising an amount of at least one compound of Formula I or a pharmaceutically acceptable salt or ester thereof, and an amount of one or more additional therapeutic agents listed above (administered together or sequentially) wherein the amounts of the compounds/treatments result in desired therapeutic effect.

When administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. The amounts of the various actives in such combination therapy may be different amounts (different dosage amounts) or same amounts (same dosage amounts). Thus, for illustration purposes, a compound of Formula I and an additional therapeutic agent may be present in fixed amounts (dosage amounts) in a single dosage unit (e.g., a capsule, a tablet and the like). A commercial example of such single dosage unit containing fixed amounts of two different active compounds is VYTORIN® (available from Merck Schering-Plough Pharmaceuticals, Kenilworth, N.J.).

If formulated as a fixed dose, such combination products employ the compounds of this invention within the dosage range described herein and the other pharmaceutically active agent or treatment within its dosage range. Compounds of Formula I may also be administered sequentially with known therapeutic agents when a combination formulation is inappropriate. The invention is not limited in the sequence of administration; compounds of Formula I may be administered either prior to or after administration of the known therapeutic agent. Such techniques are within the skills of persons skilled in the art as well as attending physicians.

The pharmacological properties of the compounds of this invention may be confirmed by a number of pharmacological assays. The antitumor activity of the compounds of the present invention (including growth suppression activity as well as the intereference in the ability of tumerigenic cells to grow in the absence of adhesion) may be assayed by methods known in the art, for example, by using the methods as described in the examples (see for example, the proliferation assay and soft agar assay in the examples)

While it is possible for the active ingredient to be administered alone, it is preferable to present it as a pharmaceutical composition. The compositions of the present invention comprise at least one active ingredient, as defined above, together with one or more acceptable carriers, adjuvants or vehicles thereof and optionally other therapeutic agents. Each carrier, adjuvant or vehicle must be acceptable in the sense of being compatible with the other ingredients of the composition and not injurious to the mammal in need of treatment.

Accordingly, this invention also relates to pharmaceutical compositions comprising at least one compound of Formula I, or a pharmaceutically acceptable salt or ester thereof and at least one pharmaceutically acceptable carrier, adjuvant or vehicle.

For preparing pharmaceutical compositions from the compounds described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

The term pharmaceutical composition is also intended to encompass both the bulk composition and individual dosage units comprised of more than one (e.g., two) pharmaceutically active agents such as, for example, a compound of the present invention and an additional agent selected from the lists of the additional agents described herein, along with any pharmaceutically inactive excipients. The bulk composition and each individual dosage unit can contain fixed amounts of the afore-said “more than one pharmaceutically active agents”. The bulk composition is material that has not yet been formed into individual dosage units. An illustrative dosage unit is an oral dosage unit such as tablets, pills and the like. Similarly, the herein-described method of treating a subject by administering a pharmaceutical composition of the present invention is also intended to encompass the administration of the afore-said bulk composition and individual dosage units.

Additionally, the compositions of the present invention may be formulated in sustained release form to provide the rate controlled release of any one or more of the components or active ingredients to optimize the therapeutic effects. Suitable dosage forms for sustained release include layered tablets containing layers of varying disintegration rates or controlled release polymeric matrices impregnated with the active components and shaped in tablet form or capsules containing such impregnated or encapsulated porous polymeric matrices.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

The compounds of this invention may also be delivered subcutaneously.

Preferably the compound is administered orally.

Preferably, the pharmaceutical preparation is in a unit dosage form. In such form, the preparation is subdivided into suitably sized unit doses containing appropriate quantities of the active component, e.g., an effective amount to achieve the desired purpose.

The quantity of active compound in a unit dose of preparation may be varied or adjusted from about 1 mg to about 100 mg, preferably from about 1 mg to about 50 mg, more preferably from about 1 mg to about 25 mg, according to the particular application.

The actual dosage employed may be varied depending upon the requirements of the patient and the severity of the condition being treated. Determination of the proper dosage regimen for a particular situation is within the skill of the art. For convenience, the total daily dosage may be divided and administered in portions during the day as required.

The amount and frequency of administration of the compounds of the invention and/or the pharmaceutically acceptable salts or esters thereof will be regulated according to the judgment of the attending clinician considering such factors as age, condition and size of the patient as well as severity of the symptoms being treated. A typical recommended daily dosage regimen for oral administration can range from about 1 mg/day to about 500 mg/day, preferably 1 mg/day to 200 mg/day, in two to four divided doses.

Another aspect of this invention is a kit comprising a therapeutically effective amount of at least one compound of Formula I or a pharmaceutically acceptable salt or ester thereof and at least one pharmaceutically acceptable carrier, adjuvant or vehicle.

Yet another aspect of this invention is a kit comprising an amount of at least one compound of Formula I or a pharmaceutically acceptable salt or ester thereof and an amount of at least one additional therapeutic agent listed above, wherein the amounts of the two or more ingredients result in desired therapeutic effect.

The invention disclosed herein is exemplified by the following preparations and examples which should not be construed to limit the scope of the disclosure. Alternative mechanistic pathways and analogous structures will be apparent to those skilled in the art.

The following solvents and reagents may be referred to by their abbreviations in parenthesis:

Thin layer chromatography: TLC

dichloromethane: CH₂Cl₂

ethyl acetate: AcOEt or EtOAc

methanol: MeOH

trifluoroacetate: TFA

triethylamine: Et₃N or TEA

butoxycarbonyl: n-Boc or Boc

nuclear magnetic resonance spectroscopy: NMR

liquid chromatography mass spectrometry: LCMS

high resolution mass spectrometry: HRMS

milliliters: mL

millimoles: mmol

microliters: μl

grams: g

milligrams: mg

room temperature or rt (ambient): about 25° C.

dimethoxyethane: DME

### EXAMPLES

Illustrating the invention are the following examples which, however, are not to be considered as limiting the invention to their details. Unless otherwise indicated, all parts and percentages in the following examples, as well as throughout the specification, are by weight.

### Preparative Example 1

### 1-(4,4′-DICHLOROBENZHYDRYL)PIPERAZINE

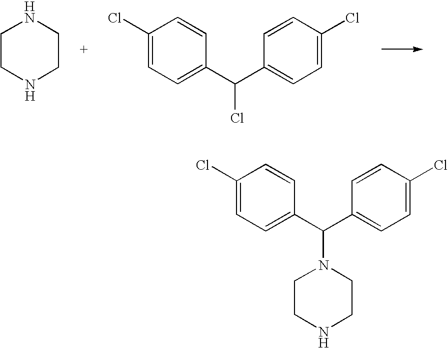

Bis-(4-chlorophenyl)methyl chloride (3 g, 9.3 mmoles) [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)] was reacted, using a modified procedure to that described in the above article, with piperazine (1.68 g, 27.9 mmoles), anhydrous potassium iodide (2.02 g, 10.23 mmoles) and anhydrous potassium carbonate (1.68 g, 10.23 mmoles) in anhydrous acetonitrile (90 mL) and the mixture was heated under reflux at 82° C. for 89 h. The resulting slurry was evaporated to dryness and the residue was partitioned between water and dichloromethane. The dichloromethane layer was dried (MgSO₄), filtered and evaporated to dryness. The residue was chromatographed on a silica gel column (30×5 cm) using 3% (10% conc. NH₄OH in methanol)-dichloromethane as the eluant to give the title compound as a pale yellow solid (2.99 g, 84%).

### Preparative Example 2

### 5-BROMO-2-[CHLORO-(2,4-DICHLOROPHENYL)METHYL]PYRIDINE

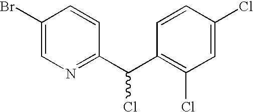

### A. 2,4-DICHLORO-N-METHOXY-N-METHYLBENZAMIDE

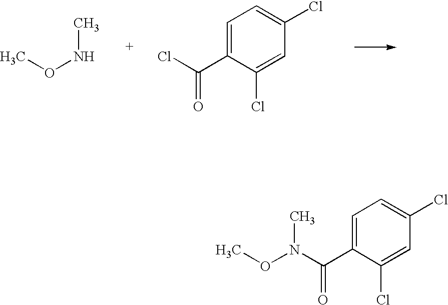

2,4-Dichlorobenzoyl chloride (11.59 g, 7.76 mL, 55.3 mmoles) and N,O-dimethylhyoxy]amine hydrochloride (4.91 g, 50.3 mmoles) were dissolved in anhydrous dichloromethane (550 mL) and the mixture was cooled to 0° C. under argon. Anhydrous pyridine (8.76 g, 8.96 mL, 110.6 mmoles) was added dropwise to the stirred solution at 0° C. and the mixture was stirred at 0° C. for 6 h. The mixture was evaporated to dryness and the residue was partitioned between diethyl ether-dichloromethane (1:1) and brine. The organic layer was dried (MgSO₄), filtered and evaporated to dryness. The residue was chromatographed on a silica gel column (60×5 cm) using 1% (10% conc. NH₄OH in methanol)-dichloromethane as the eluant to give the title compound as a colorless oil (11.78 g, 100%): ESMS: m/z 234.0 (MH⁺); Found: C, 46.16; H, 3.55; Cl, 29.81; N, 5.96. C₉H₉Cl₂NO₂ requires: C, 46.18; H, 3.88; Cl, 30.29; N, 5.98; δ_{H} (CDCl₃) 3.34 (3H, s, NCH₃), 3.48 (3H, s, OCH₃), 7.26 (2H, s, H₃, H₅) and 7.43 ppm (1H, s, H₆); δ_{C}CDCl₃) CH₃: 32.3, 61.5; CH: 127.0, 128.7, 129.6; CH: 127.0, 128.7 129.6: C: 131.8, 133.8, 135.6, 165.1.

### B. (5-BROMOPYRIDIN-2-YL)-(2,4-DICHLOROPHENYL)METHANONE

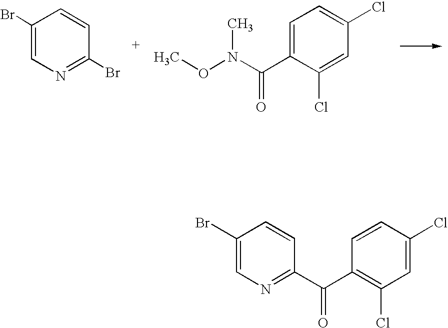

2,5-Dibromopyridine (10.2 g, 43.1 mmoles) was dissolved in anhydrous toluene (510 mL) and the mixture was stirred under argon at −78° C. 2.5M n-Butyl lithium in hexanes (20.3 mL, 51.72 mmoles) was added dropwise at −78° C. over 30 min and the mixture was stirred for 2 h at −78° C. A solution of 2,4-dichloro-N-methoxy-N-methylbenzamide (10.04 g, 43.1 mmoles) in anhydrous toluene (2 ml) was added dropwise to the stirred solution and the mixture was stirred at −78° C. for 1 h. The mixture was allowed to warm up to −10° C. Saturated aqueous NH₄Cl (102 mL) was added and the mixture was stirred and allowed to warm up to 25° C. The toluene layer was separated and dried (MgSO₄), filtered and evaporated to dryness. The residue was chromatographed on a silica gel column (30×5 cm) using 2% ethyl acetate in hexane as the eluant to give the title compound as a cream solid (9.42 g, 68%): FABMS: m/z 330 (MH⁺); HRFABMS: m/z 331.9066 (MH⁺). Calcd. for C₁₂H₇BrCl₂NO: m/z 331.9065; Found: C, 43.27; H, 1.70; Br, 23.78; Cl, 21.79; N, 4.09; C₁₂H₆BrCl₂NO requires: C, 43.54; H, 1.83; Br, 24.14; Cl, 21.42; N, 4.23; δ_{H} (CDCl₃) 7.38 (1H, dd, H_{3′}), 7.45 (2H, d, H_{5′} and H_{6′}), 8.05 (2H, dd, H₃ and H₄) and 8.70 ppm (1H, s, H₆); δ_{C} (CDCl₃) CH, 124.9, 127.2, 130.0, 131.0, 140.0, 150.6; C, 125.6, 133.1, 136.2, 137.4, 151.8, 193.5, as well as the title compound of Preparative Example 3A (380.9 mg, 3%): FABMS: m/z 330.1 (MH⁺); Found: C, 43.80; H, 1.89; Br, 23.91; Cl, 21.82; N, 4.23. C₁₂H₆BrCl₂NO requires: C, 43.54; H, 1.83; Br, 24.14; Cl, 21.42; N, 4.23; δ_{H} (CDCl₃) 7.38 (1H, s, H_{3′}), 7.42 (1H, d, H_{5′}), 7.53 (1H, d, H_{6′}), 7.64 (1H, d, H₅), 7.97 (1H, dd, H₄) and 8.64 ppm (1H, d, H₂); δ_{C} (CDCl₃) CH: 127.8, 128.6, 130.5, 130.6, 138.8, 151.7/151.8; C: 131.1, 132.6, 135.4, 138.0, 147.7, 192.1 and bis-(2,4-dichlorophenyl)methanone (412.6 mg, 3%): FABMS: m/z 424 (MH⁺).

### C. (5-BROMOPYRIDIN-2-YL)-(2,4-DICHLOROPHENYL)METHANOL

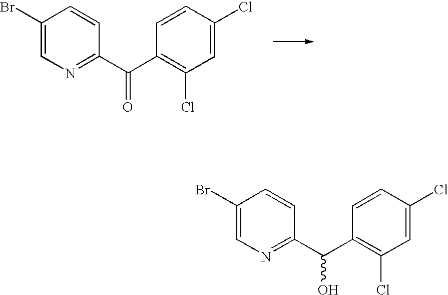

The title compound from Step A above (8.3 g, 25.1 mmoles) was dissolved in methanol (200 mL) and dichloromethane (50 mL) and cooled to 0° C. Sodium borohydride (1.38 g, 36.6 mmoles) was added and the mixture was stirred at 0° C. for 2.5 h and then allowed to warm up to 25° C. over a period of 1 h. The mixture was evaporated to dryness and the residue was partitioned between ethyl acetate and water. The ethyl acetate layer was dried (MgSO₄), filtered and evaporated to dryness. The residue was chromatographed on a silica gel column (30×5 cm) using 3-5% ethyl acetate in hexane as the eluant to give the title compound (6.93 g, 83%): FABMS: m/z 331.9 (MH⁺); δ_{H} (CDCl₃) 6.18 (1H, d, CHOH), 7.17 (1H, d, H_{6′}), 7.25 (1H, dd, H_{5′}), 7.36 (1H, d, H₃), 7.41 (1H, d, H_{3′}), 7.77 (1H, dd, H₄) and 8.63 ppm (1H, d, H₆); δ_{C} (CDCl₃) CH: 70.5, 122.6, 127.8, 129.4, 129.7, 139.8, 149.4; C: 119.9, 133.3, 134.4, 138.8, 158.2.

### D. 5-BROMO-2-[CHLORO-(2,4-DICHLOROPHENYL)METHYL]PYRIDINE

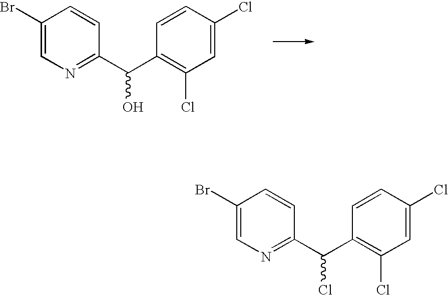

The title compound from Step D above (2.83 g, 8.57 mmoles) and triethylamine (3.58 mL, 25.7 mmoles) were added to anhydrous cyclohexane (50 mL) and the mixture was stirred at 25° C. for 15 min until all of the material had dissolved. Thionyl chloride (4.38 mL, 60 mmoles) was added and the mixture was stirred at 25° C. for 2.5 h. and then evaporated to dryness. The residue was chromatographed on a silica gel column (30×5 cm) using 2% ethyl acetate in hexane as the eluant to give 5-bromo-2-[chloro-(2,4-dichlorophenyl)methyl]pyridine (2.94 g, 98%).

### Preparative Example 3

### A. (6-BROMOPYRIDIN-3-YL)-(2,4-DICHLOROPHENYL)METHANONE

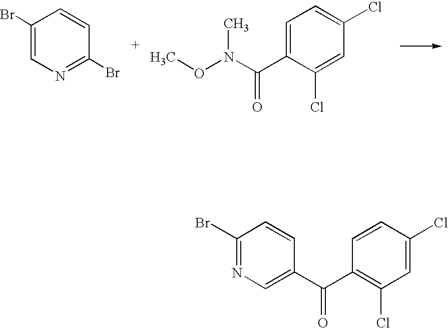

2,5-Dibromopyridine (10.8 g, 45.6 mmoles) was dissolved in anhydrous diethyl ether (541 mL) and the mixture was stirred under argon at −78° C. 2.5M n-Butyl lithium in hexanes (21.5 mL, 54.7 mmoles) was added dropwise at −78° C. over 10 min and the mixture was stirred for 40 min at −78° C. A solution of 2,4-dichloro-N-methoxy-N-methylbenzamide (10.64 g, 45.61 mmoles) (prepared as described in Preparative Example 2, Step A above) in anhydrous diethyl ether (8 ml) was added dropwise over 10 min to the stirred solution and the mixture was stirred at −78° C. for 1 h. The mixture was allowed to warm up to −10° C. Saturated aqueous NH₄Cl (108 mL) was added and the mixture was stirred and allowed to warm up to 25° C. The ether layer was separated and dried (MgSO₄), filtered and evaporated to dryness. The residue was chromatographed on a silica gel column (30×5 cm) using 2% ethyl acetate in hexane as the eluant to give the title compound as a cream solid (10.11 g, 67%): FABMS: m/z 330.1 (MH⁺); Found: C, 43.80; H, 1.89; Br, 23.91; Cl, 21.82; N, 4.23. C₁₂H₆BrCl₂NO requires: C, 43.54; H, 1.83; Br 24.14; Cl, 21.42; N, 4.23; δ_{H} (CDCl₃) 7.38 (1H, d, H_{6′}), 7.42 (1H, dd, H_{5′}), 7.53 (1H, d, H_{3′}), 7.64 (1H, d, H₅), 7.97 (1H, dd, H₄) and 8.64 ppm (1H, d, H₂); δ_{C} (CDCl₃) CH: 127.8, 128.6, 130.5, 130.6, 138.8, 151.7/151.8; C: 131.1, 132.6, 135.4, 138.0, 147.7, 192.1.

### B. (6-BROMOPYRIDIN-3-YL)-(2,4-DICHLOROPHENYL)METHANOL

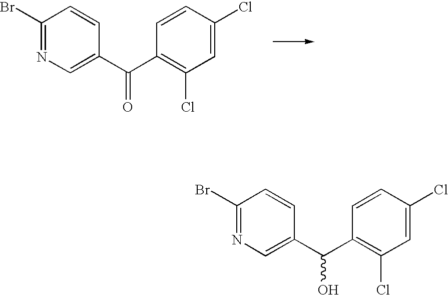

The title compound from Step A above (7.1 g, 21.5 mmoles) was dissolved in methanol (200 mL) and dichloromethane (50 mL) and cooled to 0° C. Sodium borohydride (1.18 g, 31.4 mmoles) was added and the mixture was stirred at 0° C. for 2.5 h and then allowed to warm up to 25° C. over a period of 1 h. The mixture was evaporated to dryness and the residue was partitioned between ethyl acetate and water. The ethyl acetate layer was dried (MgSO₄), filtered and evaporated to dryness. The residue was chromatographed on a silica gel column (30×5 cm) using 10% ethyl acetate in hexane as the eluant to give the title compound (6.88 g, 96%): FABMS: m/z 331.9 (MH⁺); Found: C, 43.32; H, 2.61; 23.33; Cl, 20.71: N, 3.96. C₁₂H₈BrCl₂NO requires: C, 43.28; H, 2.42; Br, 23.99; Cl, 21.29; N, 4.21; δ_{H} (CDCl₃) 6.17 (1H, d, CHOH), 7.32 (1H, d, H_{5′}), 7.38 (1H, d, H_{3′}), 7.46 (1H, d, H_{6′}), 7.52 (1H, dd, H₄), 7.57 (1H, d, H₃) and 8.36 ppm (1H, d, H₆); δ_{C} (CDCl₃) CH: 69.6, 127.9, 128.1, 128.7, 129.6, 137.2, 149.0; C: 132.8, 134.7, 137.0, 138.5, 141.4.

### C. 6-BROMO-3-[CHLORO-(2,4-DICHLOROPHENYL)METHYL]PYRIDINE

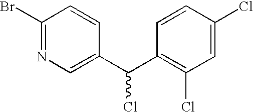

The title compound from Step B above (3 g, 8.5 mmoles) and triethylamine (2.76 g, 3.8 mL, 25.5 mmoles) were dissolved in anhydrous cyclohexane (70 mL). Thionyl chloride (7.56 g, 4.64 mL, 59.5 mmoles) was added and the mixture was heated under nitrogen at 81° C. for 4 h. The mixture was evaporated to dryness and the residue was taken up in dichloromethane and chromatographed on a silica gel column (30×5 cm) using 3% ethyl acetate in hexane as the eluant to give 6-bromo-3-[chloro-(2,4-dichlorophenyl)methyl]pyridine as a red oil (2.97 g, 96%): FABMS: m/z 350.0 (MH⁺); HRFABMS: m/z 349.8908 (MH⁺), Calcd. for C₁₂H₈BrCl₃N: m/z 349.8906; δ_{H} (CDCl₃) 6.46 (1H, s, CHCl), 7.34 (1H, dd, H_{5′}), 7.42 (1H, d, H_{3′}), 7.48 (1H, d, H_{6′}), 7.54 (1H, dd, H₄), 7.58 (1H, d, H₃) and 8.38 ppm (1H, d, H₆); δ_{C} (CDCl₃) CH: 56.5, 128.1, 128.2, 129.8, 130.5, 137.8, 149.4; C: 133.3, 134.9, 135.5, 135.5, 142.1.

### Preparative Example 4

### 5-BROMO-2-[CHLORO-(3,5-DICHLOROPHENYL)METHYL]PYRIDINE

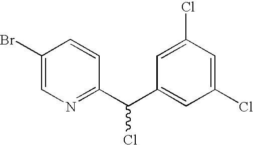

### A. 3,5-DICHLORO-N-METHOXY-N-METHYLBENZAMIDE

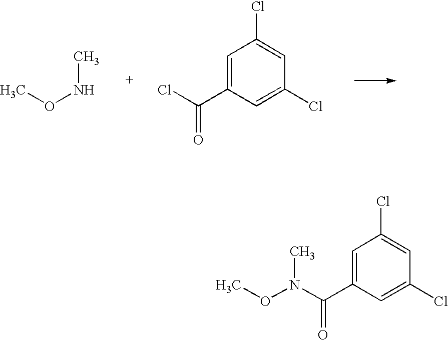

3,5-Dichlorobenzoyl chloride (10.0 g, 47.7 mmoles) and N,O-dimethylhyoxylamine hydrochloride (4.23 g, 43.4 mmoles) were dissolved in anhydrous dichloromethane (475 mL) and the mixture was cooled to 0° C. under argon. Anhydrous pyridine (7.55 g, 7.79 mL, 95.5 mmoles) was added dropwise to the stirred solution at 0° C. and the mixture was stirred at 0° C. for 5 h. The mixture was evaporated to dryness and the residue was partitioned between diethyl ether-dichloromethane (1:1) and brine. The organic layer was dried (MgSO₄), filtered and evaporated to dryness. The residue was chromatographed on a silica gel column (60×5 cm) using 0.75% (10% conc. NH₄OH in methanol)-dichloromethane as the eluant to give 3,5-dichloro-N-methoxy-N-methylbenzamide as a colorless oil (9.66 g, 95%): FABMS: m/z 234.2 (MH⁺); HRFABMS: m/z 234.0090 (MH⁺), Calcd. for C₉H₁₀Cl₂NO₂: m/z 234.0089; δ_{H} (CDCl₃) 3.34 (3H, s, NCH₃), 3.54 (3H, s, OCH₃), 7.44 (1H, dd, H₄), 7.56 ppm (2H, d, H₂ and H₆); δ_{C} (CDCl₃) CH₃: 33.5, 61.5; CH: 126.9, 126.9, 130.6; C: 134.8, 134.8, 136.7, 166.8.

### B. (5-BROMOPYRIDIN-2-YL)-(3,5-DICHLOROPHENYL)METHANONE

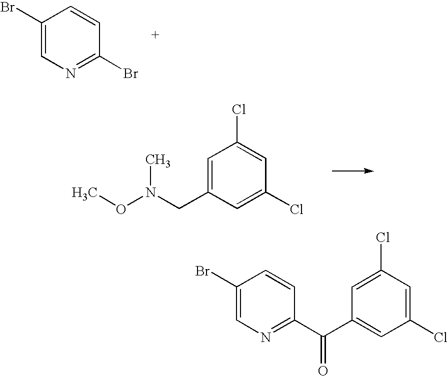

2,5-Dibromopyridine (9.21 g, 38.9 mmoles) was dissolved in anhydrous toluene (462 mL) and the mixture was stirred under argon at −78° C. 2.5M n-Butyl lithium in hexanes (18.66 mL, 46.7 mmoles) was added dropwise at −78° C. over 30 min and the mixture was stirred for 2 h at −78° C. A solution of 3,5-dichloro-N-methoxy-N-methylbenzamide (9.1 g, 38.9 mmoles) from Step A above, in anhydrous toluene (10 ml) was added dropwise to the stirred solution and the mixture was stirred at −78° C. for 1 h. The mixture was allowed to warm up to −10° C. Saturated aqueous NH₄Cl (92 mL) was added and the mixture was stirred and allowed to warm up to 25° C. The toluene layer was separated and dried (MgSO₄), filtered and evaporated to dryness. The residue was chromatographed on a silica gel column (45×8 cm) using 1% ethyl acetate in hexane as the eluant to give (5-bromopyridin-2-yl)-(3,5-dichlorophenyl)methanone as a cream solid (8.88 g, 69%): Found: C, 43.55; H, 1.88; Br, 24.13; Cl, 21.82; N, 4.22. C₁₂H₆BrCl₂NO requires: C, 43.54; H, 1.83; Br, 24.14; Cl, 21.42; N, 4.23; FABMS: m/z 331.8 (MH⁺); HRFABMS: m/z 331.9066 (MH⁺), Calcd. for C₁₂H₇BrCl₂NO: m/z 331.9065; δ_{H} (CDCl₃) 7.57 (1H, dd, H_{4′}), 7.97 (2H, d, H₂, and H_{6′}), 7.99 (1H, d, H₃), 8.07 (1H, dd, H₄) and 8.78 ppm (1H, d, H₆); δ_{C} (CDCl₃) CH: 126.1, 129.4, 129.4, 132.7, 140.2, 150.0; C: 125.4, 135.1, 135.1, 138.5, 152.1, 189.9.

### C. (5-BROMOPYRIDIN-2-YL)-(3,5-DICHLOROPHENYL)METHANOL

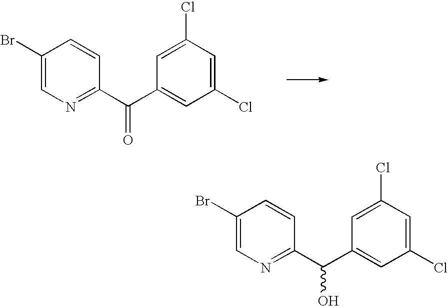

The title compound from Step B above (8.18 g, 24.6 mmoles) was dissolved in methanol (200 mL) and dichloromethane (50 mL) and cooled to 0° C. Sodium borohydride (1.35 g, 35.9 mmoles) was added and the mixture was stirred at 0° C. for 2.5 h and then allowed to warm up to 25° C. over a period of 1 h. The mixture was evaporated to dryness and the residue was partitioned between ethyl acetate and water. The ethyl acetate layer was dried (MgSO₄), filtered and evaporated to dryness. The residue was chromatographed on a silica gel column (30×5 cm) using 4% ethyl acetate in hexane as the eluant to give (5-bromopyridin-2-yl)-(3,5-dichlorophenyl)methanol (8.06 g, 99%): Found: C, 43.20; H, 2.37; Br, 23.86; Cl, 21.69; N, 4.04. C₁₂H₈BrCl₂NO requires; C, 43.28; H, 2.42; Br, 23.99; Cl, 21.29; N, 4.21; FABMS: m/z 334.0 (MH⁺); HRFABMS: m/z 333.9223 (MH⁺). Calcd. for C₁₂H₉BrCl₂NO: m/z 333.9221; δ_{H} (CDCl₃) 4.82 (1H, d, CHOH), 5.66 (1H, d, CHOH), 7.12 (1H, d, H₃), 7.27 (2H, d, H_{2′}, and H_{6′}), 7.81 (1H, dd, H₄) and 8.64 ppm (1H, d, H₆); δ_{C} (CDCl₃) CH: 73.9, 122.5, 125.4, 125.4, 128.3, 139.9, 149.5; C: 120.0, 135.3, 135.3, 146.0, 158.3.

### D. 5-BROMO-2-[CHLORO-(3,5-DICHLOROPHENYL)METHYL]PYRIDINE

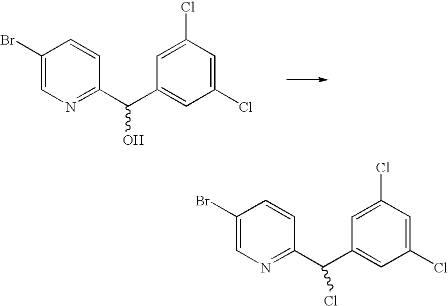

The title compound from Step C above (0.303 g, 0.91 mmoles) and triethylamine (0.276 g, 0.38 mL, 2.73 mmoles) were dissolved in anhydrous cyclohexane (12 mL). Thionyl chloride (0.764 g, 0.465 mL, 6.37 mmoles) was added and the mixture was stirred under nitrogen at 25° C. for 3.5 h. The mixture was evaporated to dryness and the residue was taken up in dichloromethane and chromatographed on a silica gel column (30×2.5 cm) using 2% ethyl acetate in hexane as the eluant to give 5-bromo-2-[chloro-(3,5-dichlorophenyl)methyl]pyridine as an oil (0.314 g, 98%): FABMS: m/z 349.9 (MH⁺); HRFABMS: m/z 351.8881 (MH⁺). Calcd. for C₁₂H₈Cl₃N: m/z 351.8881; δ_{H} (CDCl₃) 5.99 (1H, s, CHCl), 7.30 (1H, dd, H_{4′}), 7.34 (2H, d H_{2′} and H_{6′}), 7.46 (1H, d, H₃), 7.88 (1H, dd, H₄) and 8.63 ppm (1H, d, H₆); δ_{C} (CDCl₃) CH: 62.2, 123.4, 126.4, 126.4, 128.7, 140.1, 150.6; C: 120.5, 135.3, 135.3, 142.7, 157.0.

### Preparative Example 5

### 4-TRIFLUOROMETHOXYBENZHYDRYL CHLORIDE

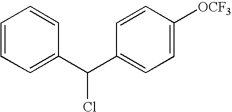

Phenyl-(4-trifluoromethoxyphenyl)methanol (463.5 mg, 17.3 mmoles) [prepared by essentially the same procedure as described in Preparative Example 4, Step C by reduction of phenyl-(4-trifluoromethoxyphenyl)methanone. The latter may be prepared as described in: J. R. Desmurs, M. Labrouillere, C. Le Roux, H. Gaspard, A. Laporterie and J. Dubac, Tetrahedron Letters, 38(15), 8871-8874 (1997)] was dissolved in anhydrous toluene (10 mL) at 0° C. Thionyl chloride (0.882 mL, 12.1 mmoles) was added and the mixture was allowed to warm up to 25° C. over a period of 18 h. The solution was evaporated to dryness and the resulting material was azeotroped with anhydrous toluene to afford 4-trifluoromethoxybenzhydryl chloride, that was used without further purification in Example 10.

### Preparative Example 6

### 2-(CHLOROMETHYL)-3H-QUINAZOLIN-4-ONE

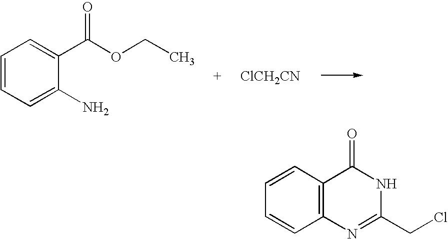

Ethyl 2-aminobenzoate (50 g, 44.76 mL, 302.7 mmoles) and chloroacetonitrile (68.56 g, 57.5 mL, 908.1 mmoles) were dissolved in anhydrous 1,4-dioxane (1L) and dry HCl gas was passed through the stirred solution at 25° C. for 5 h. The reaction was mildly exothermic for 4 h and after about 30 min the initial dense white precipitate dissolved. After about 1 h the mixture became turbid and a precipitate again formed. The reaction mixture was poured into ice/water (2L) and neutralized with concentrated ammonium hydroxide until pH 7.0 was reached. The resulting mixture was evaporated to dryness and the solid was triturated with distilled water, filtered off and rinsed with distilled water and then dried in vacuo at 50° C. for 18 h. The material was dissolved in 1,4-dioxane and silica gel was added. The mixture was then evaporated to dryness and the resulting solid was introduced onto a silica gel column (65×8.5 cm) and eluted with 3%-5%-10% methanol in dichloromethane to give 2-(chloromethyl)-3H-quinazolin-4-one (47.6 g, 81%): Found: C, 55.45; H, 3.47; N, 14.26. C₉H₇ClN₂O requires: C, 55.54; H, 3.63; N, 14.39; FABMS: m/z 195.3 (MH⁺); δ_{H} (d₆-DMSO) 4.53 (2H, s, CH₂Cl), 7.51 (1H, ddd, H₆), 7.64 (1H dd, H₈), 8.00 (1H, ddd, H₇) and 8.09 ppm (1H, dd, H₅); δ_{C} (d₆-DMSO)CH₂: 43.2; CH: 125.9, 127.2, 127.2, 134.6; C: 121.2, 148.2, 152.3, 161.5.

### Preparative Example 7

### 2-[4-{(4,4′-DICHLOROBENZHYDRYL)PIPERAZINYL}-1-METHYL]-3H-QUINAZOLIN-4-ONE

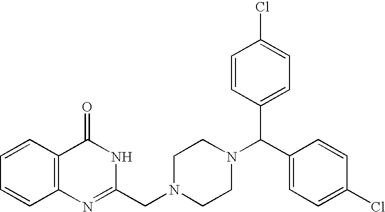

### A. 2-(PIPERAZINYL-1-METHYL)-3H-QUINAZOLIN-4-ONE and 2-[4-METHYL-2-(3H-QUINAZOLIN-4-ONE)PIPERAZIN-1-METHYL]-3H-QUINAZOLIN-4-ONE

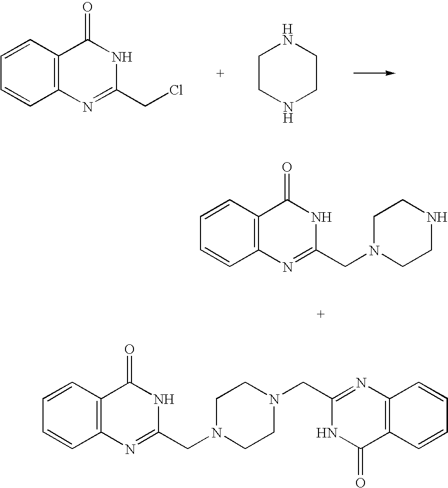

2-(Chloromethyl)-3H-quinazolin-4-one (2 g, 10.3 mmoles) (prepared as described in Preparative Example 6), piperazine (2.66 g, 30.9 mmoles) and sodium carbonate (0.54 g, 10.3 mmoles) were added to absolute ethanol (100 mL) and the slurry was heated under argon at 80° C. for 17 h. The mixture was evaporated to dryness and the resulting material was treated with methanol-dichloromethane (1:1) (500 mL) and silica gel was added. The slurry was evaporated to dryness and introduced onto a silica gel column (60×2.5 cm) and eluted with 5% (10% conc. ammonium hydroxide in methanol)-dichloromethane to give in the order of elution, 2-[4-methyl-2-(3H-quinazolin-4-one)piperazin-1-methyl]-3H-quinazolin-4-one (305.2 mg, 7%): Found: C, 62.95; H, 5.30; N, 19.51. C₂₂H₂₂N₆O₂ requires: C, 65.66; H, 5.51; N, 20.88; FABMS: m/z 403.2 (MH⁺); δ_{H} (d₆-DMSO) 2.59 (4H, s, CH₂N), 3.51 (6H, s, NCH₂CH₂N), 7.39 (2H, ddd, H₆/H_{6′}), 7.55 (2H, dd, H₈/H_{8′}), 7.68 (2H, ddd, H₇/H_{7′}) and 8.12 ppm (1H, dd, H₅/H_{5′}) and 2-(piperazinyl-1-methyl)-3H-quinazolin-4-one (2.1 g, 84%): Found: C, 63.22; H, 6.51; N, 22.53. C₁₃H₁₆N₄O requires: C, 63.91; H, 6.60; N, 22.93; FABMS: m/z 245.3 (MH⁺); δ_{H} (d₆-DMSO) 2.38 (4H, s, CH₂NCH₂), 2.68 (4H, s, CH₂NHCH₂), 3.36 (2H, s, CH₂N), 7.47 (1H, ddd, H₆), 7.61 (1H, dd, H₈), 7.77 (1H, ddd, H₇), and 8.08 ppm (1H, dd, H₅); δ_{C} (d₆-DMSO)CH₂: 45.5, 45.5, 54.0, 54.0, 61.4; CH: 125.8, 126.4, 127.0, 134.4; C: 121.3, 148.4, 154.3, 161.6.

### B. 2-[4-{(4,4′-DICHLOROBENZHYDRYL)PIPERAZINYL}-1-METHYL]-3H-QUINAZOLIN-4-ONE

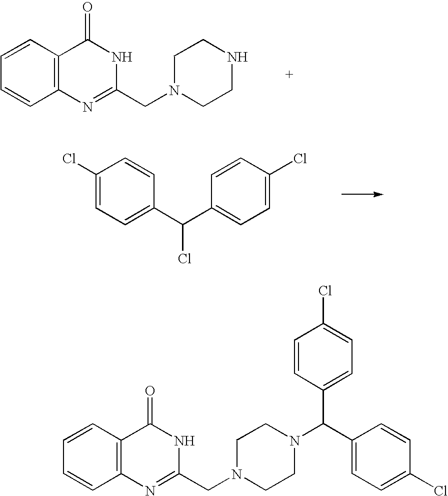 Method 1:

2-(piperazinyl-1-methyl)-3H-quinazolin-4-one (500 mg, 2.0 mmoles) (prepared as described in Preparative Example 7, Method 1, Step A above), bis-(4-chlorophenyl)methyl chloride (667 mg, 2.4 mmoles) [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)], anhydrous potassium carbonate (339.5 mg, 2.4 mmoles) and anhydrous potassium iodide (407.7 mg, 2.4 mmoles) were added to anhydrous acetonitrile (15 mL) and the mixture was heated under reflux and under argon at 82° C. for 19 h. The reaction mixture was evaporated to dryness and the residue was partitioned between dichloromethane and saturated aqueous sodium bicarbonate. The organic layer was washed with water, dried (MgSO₄), filtered and evaporated to dryness. The residue was chromatographed on a silica gel column (60×2.5 cm) using 0.5% (conc. ammonium hydroxide in methanol)-dichloromethane as the eluant to give 2-[4-{(4,4′-dichlorobenzhydryl)piperazinyl}-1-methyl]-3H-quinazolin-4-one (446.7 mg, 46%): Found: C, 64.40; H, 5.06; Cl, 15.62: N, 11.48. C₂₆H₂₄Cl₂N₄O requires: C, 65.14; H, 5.05; Cl, 14.79; N, 11.69; FABMS: m/z 479.4 (MH⁺); δ_{H} (CDCl₃) 2.44 (4H, bs, CH₂NCH₂), 2.63 (4H, bs, CH₂NCH₂), 3.58 (2H, s, 2-CH₂N), 4.24 (1H, s, NCH(C₆H₄Cl)₂), 7.25 (4H, dd, H₃/H₅/H_{3′}/H_{5′}), 7.32 (4H, d, H₂/H₆/H_{2′}/H_{6′}), 7.47 (1H, ddd, H₆), 7.66 (1H, dd, H₈), 7.76 (1H, ddd, H₇) and 8.27 ppm (1H, d, H₅); δ_{C} (CDCl₃) CH₂: 51.6, 51.6, 53.6, 53.6, 60.4; CH: 74.5, 126.7, 126.8, 127.1, 129.0, 129.0, 129.0, 129.0, 129.2, 129.2, 129.2, 129 2, 134.8; C: 121.8, 133.1, 133.1, 140.4, 140.4 149.0, 153.4 and 161.7.

Method 2:

2-(piperazinyl-1-methyl)-3H-quinazolin-4-one (1.18 g, 4.8 mmoles) (prepared as described in Preparative Example 7, Method 1, Step A above), bis-(4-chlorophenyl)methyl chloride (2.63 g, 9.6 mmoles) [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)] and anhydrous potassium carbonate (734.4 mg, 5.28 mmoles) were added to anhydrous acetonitrile (100 mL) and the mixture was heated under reflux and under argon at 82° C. for 51 h. The reaction mixture was evaporated to dryness and the residue was partitioned between dichloromethane and saturated aqueous sodium bicarbonate. The organic layer was washed with water, dried (MgSO₄), filtered and evaporated to dryness. The residue was chromatographed on a silica gel column (30×5 cm) using 0.5% (conc. ammonium hydroxide in methanol)-dichloromethane as the eluant to give 2-[4-{(4,4′-dichlorobenzhydryl)piperazinyl}-1-methyl]-3H-quinazolin-4-one (1.22 g, 52%).

Method 3:

### 2-[4-{(4,4′-DICHLOROBENZHYDRYL)PIPERAZINYL}-1-METHYL]-3H-QUINAZOLIN-4-ONE

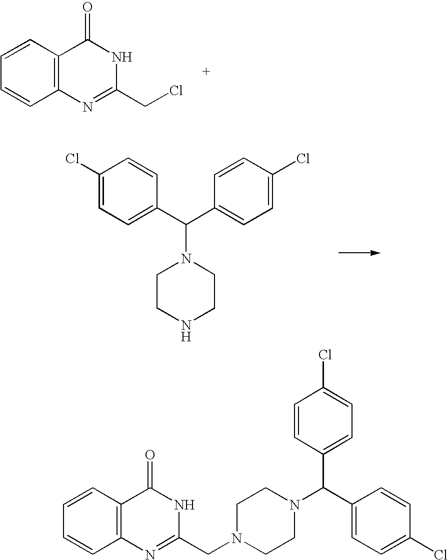

2-(Chloromethyl)-3H-quinazolin-4-one (1.18 g, 6.1 mmoles) (prepared as described in Preparative Example 6), 1-(4,4′-dichlorobenzhydryl)piperazine (2.99 g, 6.1 mmoles) (prepared as described in Preparative Example 1) and anhydrous potassium carbonate (1.42 g, 6.71 mmoles) were added to anhydrous acetonitrile (100 mL) and the mixture was stirred under reflux at 80° C. and under argon for 18 h. The mixture was evaporated to dryness and the residue was partitioned between dichloromethane and saturated aqueous sodium bicarbonate. The organic layer was washed with water, dried (MgSO₄), filtered and evaporated to dryness. The residue was chromatographed on a silica gel column (30×5 cm) using 0.5% (conc. ammonium hydroxide in methanol)-dichloromethane as the eluant to give 2-[4-{(4,4′-dichlorobenzhydryl)piperazinyl}-1-methyl]-3H-quinazolin-4-one (2.56 g, 57%).

### Preparative Example 8

### 2-{4-[BIS-(4-CHLOROPHENYL)METHYL]PIPERAZIN-1-YLMETHYL}-4-CHLOROQUINAZOLINE

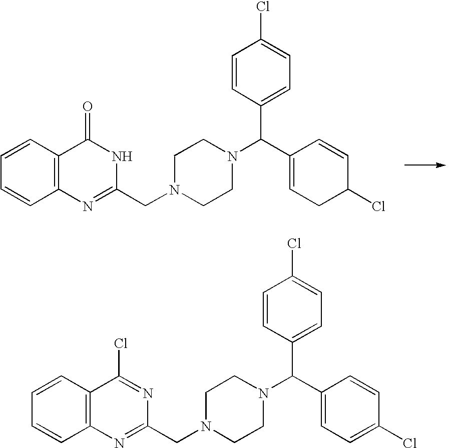 Method 1:

2-[4-{(4,4′-Dichlorobenzhydryl)piperazinyl}-1-methyl]-3H-quinazolin-4-one (200 mg, 0.417 mmoles) (prepared as described in Preparative Example 7) was added to anhydrous dichloromethane (5 mL) and thionyl chloride (496.3 mg, 0.304 mL, 4.17 mmoles) was added to the slurry. Anhydrous DMF (18.9 mg, 0.020 mL, 0.258 mmoles) was added and the mixture was heated under argon and under reflux at 80° C. for 3 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column 30×1.5 cm) using 20%-40%-50% ethyl acetate in hexane as the eluant to give 2-{4-[bis-(4-chlorophenyl)methyl]piperazin-1-ylmethyl}4-chloroquinazoline (119.7 mg, 58%): Found: C, 60.05; H, 4.80; Cl, 22.12: N, 10.28. C₂₆H₂₃Cl₃N₄ requires: C, 62.73; H, 4.66; Cl, 21.36; N, 11.25; FABMS: m/z 497.5 (MH⁺); δ_{H} (CDCl₃) 2.69 (4H, bs, CH₂NCH₂), 3.08 (4H, bs, CH₂NCH₂), 4.23 (2H, bs, CH₂N), 4.32 (1H, bs, NCH(C₆H₄Cl)₂), 7.23 (4H, dd, H₃/H₅/H_{3′}/H_{5′}), 7.31 (4H, d, H₂/H₆/H_{2′}/H_{6′}), 7.77 (1H, ddd, H₆), 7.98 (1H, ddd, H₇), 8.09 (1H, dd, H₅), and 8.29 ppm (1H, dd, H₈); δ_{C} (CDCl₃) δ1.6, 51.6, 53.6, 53.6, 64.7; CH: 74.7, 125.8, 128.6, 128.8, 128.8, 128.8, 128.8, 128.8, 129.2, 129.2, 129.2, 129.2, 134.9; C: 122.5, 132.8, 132.8, 140.9, 140.9, 140.9, 151.4, 162.6.

When the reaction was scaled up using 4 g, or 4.75 g of 2-[4-{(4,4′-dichlorobenzhydryl)piperazinyl}-1-methyl]-3H-quinazolin-4-one under the same conditions as described above, the yields of the title compound were (1.47 g, 35%) and (1.63 g, 33%) respectively.

The catalytic Vilsmeier reagent used above was developed in the nucleoside area by: J. Zemlicka and F. Sorm, Collection Czechoslov. Chem. Commun., 30, 2052-2067 (1965).

Method 2:

2-[4-{(4,4′-Dichlorobenzhydryl)piperazinyl}-1-methyl]-3H-quinazolin-4-one (200 mg, 0.417 mmoles) (prepared as described in Preparative Example 7) was treated with phosphorus oxychloride (639.7 mg, 0.389 mL, 4.17 mmoles) and the slurry was heated under argon and under reflux at 110° C. for 3 h. The mixture solidified after 30 min. N,N-Dimethylaniline (50.55 mg, 0.0529 mL, 0.417 mmoles) was added after 3 h and the mixture was heated at 110° C. for 1 h. The excess phosphorus oxychloride was removed in vacuo on a rotary evaporator and the solid was dried in vacuo for 17 h. The solid was then dissolved in dichloromethane and washed with saturated aqueous sodium bicarbonate. The organic layer was dried (MgSO₄), filtered and evaporated to dryness. The residue was chromatographed on a silica gel column (30×2.5 cm) using 10%-30% ethyl acetate in hexane as the eluant to give 2-{4-[bis-(4-chlorophenyl)methyl]piperazin-1-ylmethyl}-4-chloroquinazoline (68.5 mg, 33%).

The methodology used above was described by: I. Merino, A. Monge, M. Font, J. J. Martinez de Irujo, E. Alberdi, E Santiago, I. Prieto, J. J. Lasarte, P. Sarobe and F. Borras, II Farmaco, 54, 255-264 (1999).

### Preparative Example 9

### 2(S)-(+)-(2-CHLOROMETHYLQUINAZOLIN-4-YLAMINO)-3-METHYLBUTYRIC ACID METHYL ESTER

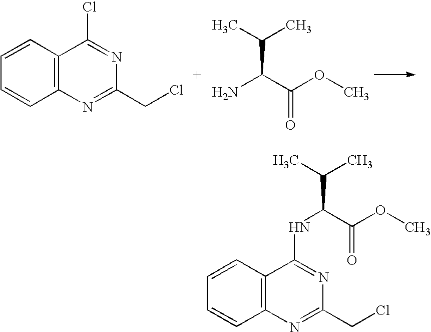

4-Chloro-2-chloromethylquinazoline (20 g, 93.9 mmoles) [prepared as described by: C. J. Shishoo, M. B. Devani, V. S. Bhadti, K. S. Jain and S. Anathan, J. Heterocyclic Chem., 27, 119-126 (1990)], L-(+)-valine methyl ester hydrochloride (15.74 g, 93.9 mmoles) and potassium carbonate (14.28 g, 103.3 mmoles) were added to anhydrous acetonitrile (700 mL) and the mixture was heated under reflux and under nitrogen at 80° C. for 18 h. The mixture was evaporated to dryness and the residue was partitioned between dichloromethane and saturated aqueous sodium bicarbonate. The organic layer was dried (MgSO₄), filtered and evaporated to dryness. The residue was chromatographed on a silica gel column (60×8.5 cm) using 2% (10% concentrated ammonium hydroxide in methanol)-dichloromethane as the eluant to give 2(S)-(+)-(2-chloromethylquinazolin-4-ylamino)-3-methylbutyric acid methyl ester (23.54 g, 81%): FABMS: m/z 308.2 (MH⁺); HRFABMS: m/z 308.1161 (MH⁺). Calcd. for C₁₅H₁₉ClN₃O₂: m/z 308.1166; δ_{H} (CDCl₃) 1.03 (3H, d, CH(CH₃)₂), 1.08 (3H, d, CH(CH₃)₂), 2.37 (3H, dq, CH(CH₃)₂), 3.81 (3H, s, COOCH₃), 4.61 (2H, s, CH₂Cl), 5.08 (1H, dd, CHCH(CH₃)₂), 6.42 (1H, d, NH), 7.44 (1H, ddd, H₆), 7.73 (1H, ddd, H₇), 7.77 (1H, dd, H₅) and 7.80 ppm (1H, dd, H₈); δ_{C} (CDCl₃) CH₃: 18.6, 19.1, 58.6; CH₂: 48.2; CH: 31.5, 52.4, 120.6, 126.5, 128.5, 133.0; C: 113.5, 149.9, 159.8, 161.3, 173.4; [α]_{D}^{25° C.} −22.7° (c=0.51, MeOH).

### Preparative Example 12

### 2(S)-(−)-(2-CHLOROMETHYLQUINAZOLIN-4-YLAMINO)-3-METHYLBUTYRAMIDE

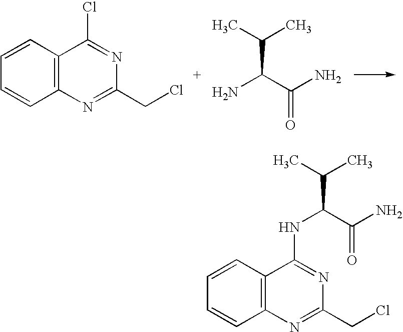

4-Chloro-2-chloromethylquinazoline (30 g, 97.5 mmoles) [prepared as described by: C. J. Shishoo, M. B. Devani, V. S. Bhadti, K. S. Jain and S. Anathan, J. Heterocyclic Chem., 27, 119-126 (1990), L-(+)-valinamide hydrochloride (21.5 g, 140.9 mmoles) and potassium carbonate (42.8 g, 309.7 mmoles) were added to anhydrous acetonitrile (500 mL) and the mixture was heated under reflux and under argon at 80° C. for 24 h. The mixture was evaporated to dryness and the residue was partitioned between dichloromethane and water. The organic layer was dried (MgSO₄), filtered and evaporated to dryness. The residue was chromatographed on a silica gel column (60×8.5 cm) using 2% (10% concentrated ammonium hydroxide in methanol)-dichloromethane as the eluant to give 2(S)-(−)-(2-aminomethylquinazolin-4-ylamino)-3-methylbutyramide (22.34 g, 78%): FABMS: m/z 293.0 (MH⁺); HRFABMS: m/z 293.1166 (MH⁺). Calcd. for C₁₄H₁₈ClN₄O: m/z 293.1169; δ_{H} (CD₃OD) 1.09 (6H, d, CH(CH₃)₂), 2.12 (3H, dq, CH(CH₃)₂), 4.57 (2H, s, CH₂Cl), 4.77 (1H, d, CHCH(CH₃)₂), 4.87 (3H, s, NH and NH₂), 7.49 (1H, ddd, H₆), 7.68 (1H, ddd, H₇), 7.75 (1H, dd, H₅) and 8.18 ppm (1H, dd, H₈); δ_{C} (CD₃OD) CH₃: 19.6, 19.6; CH₂: obscured under MeOH; CH: 31.5, 61.7, 123.4, 127.7, 127.7, 134.4; C: 114.8, 150.4, 162.1, 163.1, 177.0; [α]_{D}^{25° C.} −10.6° (c=1.01, MeOH).

### Preparative Example 13

### 2(S)-(−)-(2-CHLOROMETHYLQUINAZOLIN-4-YLAMINO)-3(R)-METHYLPENTANAMIDE and 2-CHLOROMETHYL-4-ETHOXYQUINAZOLINE

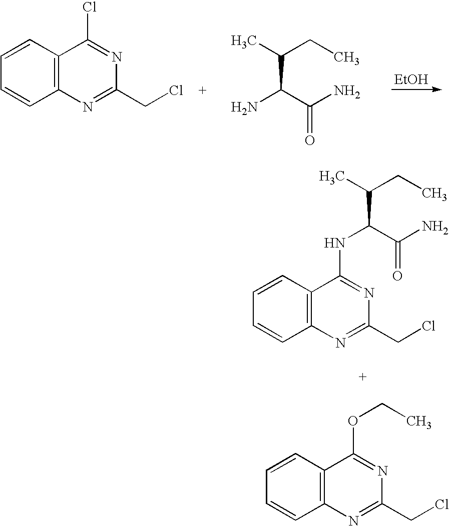

4-Chloro-2-chloromethylquinazoline (6.39 g, 20.76 mmoles) [prepared as described by: C. J. Shishoo, M. B. Devani, V. S. Bhadti, K. S. Jain and S. Anathan, J. Heterocyclic Chem., 27, 119-126 (1990), L-(+)-isoleucinamide hydrochloride (5 g, 20.76 mmoles) and potassium carbonate (4.56 g, 20.76 mmoles) were added to anhydrous acetonitrile (250 mL) and the mixture was heated under reflux and under argon at 80° C. for 25 h. Additional potassium carbonate (4.56 g, 20,76 mmoles) was added together with absolute ethanol (50 mL) and the slurry was stirred at 80° C. for a total of 46 h. The mixture was evaporated to dryness and the residue was partitioned between dichloromethane and saturated aqueous sodium bicarbonate. The organic layer was dried (MgSO₄), filtered and evaporated to dryness. The residue was chromatographed on a silica gel column (60×5 cm) using 2% (10% concentrated ammonium hydroxide in methanol)-dichloromethane as the eluant to give 2-chloromethyl-4-ethoxyquinazoline (452.3 mg, 7%): Found: C, 59.27; H, 5.01; Cl, 15.93; N, 12.58. C₁₁H₁₁ClN₂O requires: C, 59.33, H, 4.98; Cl, 15.92; N, 12.58; FABMS: m/z 222.9 (MH⁺); δ_{H} (CDCl₃) 1.52 (3H, dd, OCH₂CH₃), 4.63 (2H, q, OCH₂CH₃), 4.72 (2H, d, CH₂Cl), 7.55 (1H, ddd, H₆), 7.81 (1H, ddd, H₇) 7.90 (1H, dd, H₅) and 8.14 ppm (1H dd, H₈); δ_{C} (CDCl₃) CH₃: 14.4; CH₂: 47.8, 63.5; CH: 123.6, 127.3, 127.6, 133.8; C: 115.4, 151.0, 161.2, 167.5 and 2(S)-(−)-(2-chloromethylquinazolin-4-ylamino)-3(R)-methylpentanamide (5.33 g, 58%): FABMS: m/z 307.0 (MH⁺); HRFABMS: m/z 307.1323 (MH⁺). Calcd. for C₁₅H₂₀ClN₄O: m/z 307.1326; δ_{H} (CDCl₃) 0.93 (3H t, CH₃CHCH₂CH₃), 1.05 (3H, d, CH₃CHCH₂CH₃), 1.30 (1H, dq, CH₃CHCH₂CH₃), 1.70 (1H, dq, CH₃CHCH₂CH₃), 2.24 (1H, ddq, CH₃CHCH₂CH₃), 4.62 (2H, s, —CH₂Cl), 4.73 (1H, dd, NHCHCONH₂), 6.11 (1H, bs, NH), 6.57 (1H, bs NH₂), 7.24 (1 h, bs, NH₂), 7.34 (1H, ddd, H₆), 7.64 (1H, ddd, H₇), 7.70 (1H, dd, H₅) and 7.77 ppm (1H, dd, H₈); δ_{C} (CDCl₃) CH₃: 11.0, 15.7; CH₂: 25.5, 47.9; CH: 35.9, 59.1, 121.2, 126.7, 127.6, 133.3; C: 113.4, 148.5, 160.0, 160.7, 174.6; [α]_{D}²⁵° C. −18.9° (c=0.53, MeOH).

### Preparative Example 14

### 2(S)-(+)-(2-CHLOROMETHYLQUINAZOLIN-4-YLAMINO)-4-METHYLPENTANAMIDE

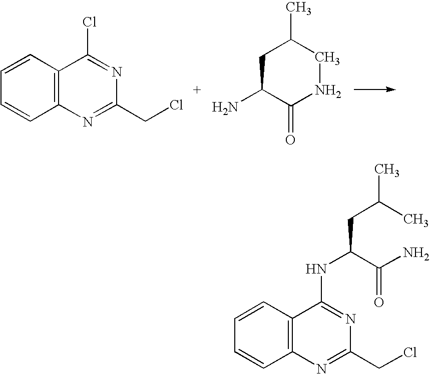

4-Chloro-2-chloromethylquinazoline (2 g, 6.5 mmoles) [prepared as described by: C. J. Shishoo, M. B. Devani, V. S. Bhadti, K. S. Jain and S. Anathan, J. Heterocyclic Chem., 27, 119-126 (1990), L-(+)-leucinamide hydrochloride (1.56 g, 6.5 mmoles) and potassium carbonate (1.43 g, 6.5 mmoles) were added to anhydrous acetonitrile (50 mL) and the mixture was heated under reflux and under argon at 80° C. for 18 h. The mixture was evaporated to dryness and the residue was partitioned between dichloromethane and water. The organic layer was dried (MgSO₄), filtered and evaporated to dryness. The residue was chromatographed on a silica gel column (30×5 cm) using 2% (10% concentrated ammonium hydroxide in methanol)-dichloromethane as the eluant to give 2(S)-(+)-(2-chloromethylquinazolin-4-ylamino)-4-methylpentanamide (1.69 g, 59%): FABMS: m/z 307.1 (MH⁺), HRFABMS: m/z 307.1321 (MH⁺). Calcd. for C₁₅H₂₀ClN₄O: m/z 307.1326; δ_{H} (CDCl₃) 0.97 (3H, s, CH(CH₃)₂), 1.02 (3H, s, CH(CH₃)₂), 1.83 (1H, m, CH₂CH(CH₃)₂), 1.92 (2H, m, CH₂CH(CH₃)₂), 4.63 (2H, m, CH₂Cl), 4.97 (1H, m, NHCHCONH₂), 5.84 (1H, bs, NH), 6.74 (1H, bs, CONH₂), 6.91 (1H, bs, CONH₂), 7.32 (1H, m, H₆), 7.66 ppm (3H, m, H_{5′} H₇ and H₈); δ_{C} (CDCl₃) CH₃: 22.3, 23.1; CH₂: 44.4, 48.4; CH: 25.0, 52.7, 120.9, 126.6, 128.1, 133.1; C: 113.4, 149.4, 159.9, 160.7, 175.4; [α]_{D}^{25° C.} +5.4° (c=0.53, MeOH).

### Preparative Example 21

### 3-METHYL-2(S)-(−)-[(2-PIPERAZIN-1-YLMETHYL)QUINAZOLIN-4-YLAMINO]BUTYRIC ACID METHYL ESTER and 2(S)-(−)-(2-{4-[4-(1-METHOXYCARBONYL-2-METHYLPROPYLAMINO)QUINAZOLIN-2-YLMETHYL]PIPERAZINYL-1-YLMETHYL}QUINAZOLIN-4-YLAMINO)-3-METHYLBUTYRIC ACID METHYL ESTER

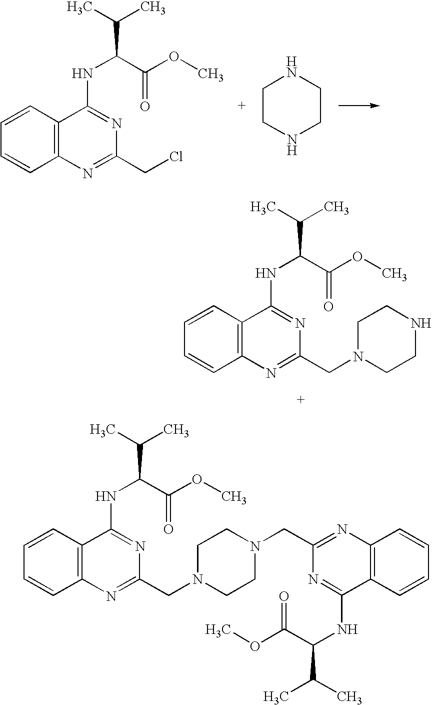

2(S)-(+)-(2-Chloromethylquinazolin-4-ylamino)-3-methylbutyric acid methyl ester (8 g, 27.7 mmoles) (prepared as described in Preparative Example 9), piperazine (13.44 g, 166.5 mmoles) and anhydrous potassium carbonate (3.95 g, 30.5 mmoles) were added to anhydrous acetonitrile (400 mL) and the mixture was heated under nitrogen and under reflux at 80° C. for 18 h. The mixture was evaporated to dryness and the residue was partitioned between dichloromethane and water. The organic layer was washed with water, dried (MgSO₄), filtered and evaporated to dryness. The residue was chromatographed on two silica gel columns (60×5 cm) using 4% (10% concentrated ammonium hydroxide in methanol)-dichloromethane as the eluant to give in the order of elution, 2(S)-(−)-(2-{4-[4-(1-methoxycarbonyl-2-methyl-propylamino)quinazolin-2-ylmethyl]piperazinyl-1-ylmethyl}quinazolin-4-ylamino)-3-methylbutyric acid methyl ester (734.5 mg, 4%): FABMS: m/z 629.3 (MH⁺); HRFABMS: m/z 629.3574 (MH⁺). Calcd. for C₃₄H₄₅N₈O₄: m/z 629.3564; δ_{H} (CDCl₃) 0.98 (6H, d, CH(CH₃)₂), 1.04 (6H, d, CH(CH₃)₂), 2.32 (2H, dq, CH(CH₃)₂), 2.77 (8H, bs, N(CH₂CH₂)₂N), 3.73 (4H, d, 2-CH₂N), 3.75 (6H, s, COOCH₃), 5.07 (2H, dd, NHCHCOOCH₃), 6.17 (2H, d, NHCHCOOCH₃), 7.41 (2H, ddd, H₆), 7.68 (2H, ddd, H₇), 7.76 (2H, dd, H₅) and 8.02 ppm (2H, dd, H₈); δ_{C} (CDCl₃) CH₃: 18.5, 18.5, 19.0, 19.0, 58.4, 58.4; CH₂: 53.4, 53.4, 53.4, 53.4, 65.4, 65.4; CH: 31.5, 31.5, 52.3, 52.3, 120.5, 120.5, 125.7, 125.7, 128.6, 128.6, 132.6, 132.6; C: 113.4, 113.4, 150.1, 150.1, 159.1, 159.1, 162.9, 162.9, 173.2, 173.2; [α]_{D}^{25° C.} 36.9° (c=0.48, MeOH) and then 3-methyl-2(S)-(−)-[(2-piperazin-1-ylmethyl)quinazolin-4-ylamino]butyric acid methyl ester (7.01 g, 74%): FABMS: m/z 358.1 (MH⁺); HRFABMS: m/z 358.2249 (MH⁺). Calcd. for C₁₉H₂₈N₅O₂: m/z 358.2243; δ_{H} (CDCl₃) 1.00 (3H, d, CH(CH₃)₂), 1.05 (3H, d, CH(CH₃)₂), 1.99 (1H, bs, N(CH₂CH₂)₂NH), 2.34 (1H, dq, CH(CH₃)₂), 2.62 (4H, bs, N(CH₂CH₂)₂NH), 2.94 (4H, bs, N(CH₂CH₂)₂NH), 3.70 (2H, d, 2-CH₂N), 3.77 (3H, s, COOCH₃), 5.07 (1H, dd, NHCHCOOCH₃), 6.18 (1H, d, NHCHCOOCH₃), 7.23 (1H, ddd, H₆), 7.70 (1H, ddd, H₇), 7.77 (1H, dd, H₅) and 8.04 ppm (1H, dd, H₈); δ_{C} (CDCl₃) CH₃: 18.5, 19.0, 58.4; CH₂: 46.1, 46.1, 54.8, 54.8, 66.0; CH: 31.5, 52.3, 120.5, 125.7, 128.6, 132.6; C: 113.4, 159.1, 162.9, 173.2; [α]_{D}^{25° C.} −21.6° (c=0.51, MeOH).

### Preparative Example 22

### 3-METHYL-2(S)-(−)-(2-PIPERAZIN-1-YLMETHYLQUINAZOLIN-4-YLAMINO)BUTYRAMIDE and 2(S)-(−)-(2-{4-[4-(1-CARBAMOYL-2-METHYLPROPYLAMINO)QUINAZOLIN-2-YLMETHYL]-PIPERAZIN -1-YLMETHYL}QUINAZOLIN-4-YLAMINO)-3-METHYLBUTYRAMIDE

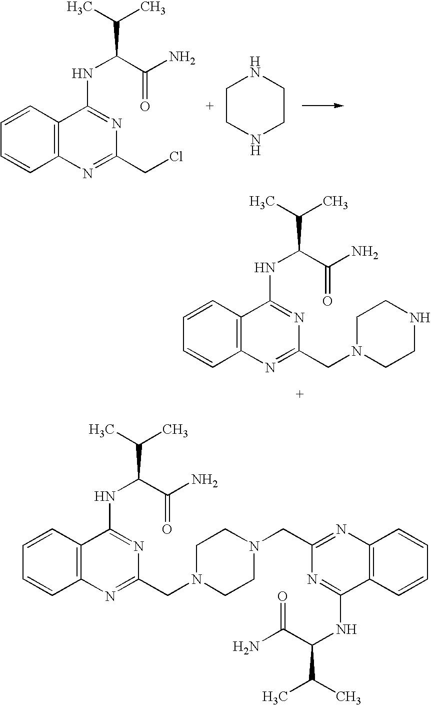

2(S)-(−)-(2-Chloromethylquinazolin-4-ylamino)-3-methylbutyramide (8 g, 27.3 mmoles) (prepared as described in Preparative Example 12), piperazine (14.16 g, 164 mmoles) and anhydrous potassium carbonate (4.16 g, 30.1 mmoles) were added to anhydrous acetonitrile (500 mL) and the mixture was heated under nitrogen and under reflux at 80° C. for 18 h. The mixture was evaporated to dryness and the residue was partitioned between dichloromethane and water. The organic layer was washed with water, dried (MgSO₄), filtered and evaporated to dryness. The residue was chromatographed on a silica gel column (60×5 cm) using 8% (10% concentrated ammonium hydroxide in methanol)-dichloromethane as the eluant to give in the order of elution, 2(S)-(−)-(2-{4-[4-(1-carbamoyl-2-methylpropylamino)quinazolin-2-ylmethyl]-piperazin-1-ylmethyl}quinazolin-4-ylamino)-3-methylbutyramide (1.34 g, 9%): FABMS: m/z 599.4 (MH⁺); HRFABMS: m/z 599.3565 (MH⁺). Calcd. for C₃₂H₄₃N₁₀O₂: m/z 599.3570; δ_{H} (CDCl₃) 1.00 (12H, d, CH(CH₃)₂), 2.33 (2H, dq, CH(CH₃)₂), 2.65 (4H, bs, N(CH₂CH₂)₂N), 2.72 (4H, bs, N(CH₂CH₂)₂N), 3.68 (4H, bs, 2-CH₂N), 4.70 (2H, dd, NHCHCONH₂), 6.74 (2H, m, NHCHCONH₂), 6.78 (2H, bs, NHCHCONH₂), 7.30 (1H, bs, NHCHCONH₂), 7.30 (2H, ddd, H₆), 7.62 (2H, ddd, H₇), 7.73 (2H, dd, H₅) and 7.79 ppm (2H, dd, H₈); δ_{C} (CDCl₃) CH₃: 19.0, 19.0, 19.6, 19.6; CH₂: 53.0, 53.0, 53.0, 53.0, 65.2, 65.2: CH: 30.1, 30.1, 60.0, 60.0, 121.0, 121.0, 125.8, 125.8, 128.1, 128.1, 132.8, 132.8; C: 113.6, 113.6, 149.8, 149.8, 159.6, 159.6, 162.9, 162.9, 175.1, 175.1; [α]_{D}^{25° C.} −1.40 (c=0.52, MeOH) and then 3-methyl-2(S)-(−)-(2-piperazin-1-ylmethylquinazolin-4-ylamino)butyramide (7.12 g, 76%): FABMS: m/z 343.1 (MH⁺); HRFABMS: m/z 343.2248 (MH⁺). Calcd. for C₁₈H₂₇N₆O: m/z 343.2246; δ_{H} (CDCl₃) 1.03 (6H, d, CH(CH₃)₂), 2.36 (1H, dq, CH(CH₃)₂), 2.44 (1H, bs, N(CH₂CH₂)₂NH), 2.57 (2H, m, N(CH₂CH₂)₂NH), 2.62 (2H, m, N(CH₂CH₂)₂NH), 2.88 (4H, bs, N(CH₂CH₂)₂NH), 3.63/3.73 (2H, AB system, 2-CH₂N), 4.78 (1H, dd, NHCHCONH₂), 6.05 (1H, bs, NHCHCONH₂), 6.69 (1H, d, NHCHCONH₂), 7.38 (1H, ddd, H₆), 7.43 (1H, bs, NHCHCONH₂), 7.68 (1H, ddd, H₇), 7.77 (1H, dd, H₅) and 8.01 ppm (1H, dd, H₈); δ_{C} (CDCl₃) CH₃: 19.0, 19.5; CH₂: 45.9, 45.9, 54.5, 54.5, 66.1; CH: 30.1, 59.7, 121.0, 125.8, 128.2, 132.8; C: 113.6, 149.9, 159.6, 162.7, 174.6; [α]_{D}^{25° C.} −11.9° (c=0.51, MeOH).

### Preparative Example 23

### 2-(4-BENZYLPIPERAZIN-1-YLMETHYL)-3H-QUINAZOLIN-4-ONE

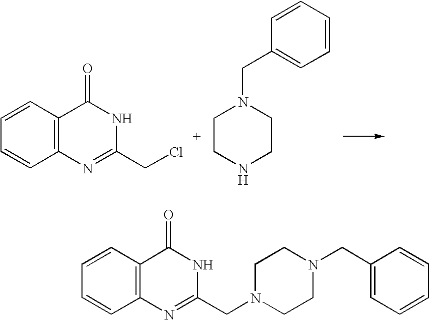 Method 1:

2-(Chloromethyl)-3H-quinazolin-4-one (30 g, 154.1 mmoles) (prepared as described in Preparative Example 6), 1-N-benzylpiperazine (80.4 mL, 462.4 mmoles) and anhydrous potassium carbonate (21 g, 154.1 mmoles) were added to anhydrous acetonitrile (1.5 L) and the mixture was heated under argon and under reflux at 80° C. for 20 h. The mixture was filtered and the solids were washed with acetonitrile and the combined filtrates were evaporated to dryness. The residue was taken up in dichloromethane and washed with saturated aqueous sodium bicarbonate. The organic layer was dried (MgSO₄), filtered and evaporated to dryness. The residue was chromatographed on a silica gel column (65×9 cm) using 0.5%-1.5%-2% (10% concentrated ammonium hydroxide in methanol)-dichloromethane as the eluant to give 2-(4-benzylpiperazin-1-ylmethyl)-3H-quinazolin-4-one (44.6 g, 87%): FABMS: m/z 335.2 (MH⁺); HRFABMS: m/z 335.1875 (MH⁺). Calcd. for C₂₀H₂₂N₄O: m/z 335.1872; Found: C, 71.55; H, 6.55; N, 16.62. C₂₀H₂₂N₄O requires: C, 71.83; H, 6.63; N, 16.75; δ_{H} (CDCl₃) 2.53 (4H, bs, N(CH₂CH₂)NBn), 2.62 (4H, bs, N(CH₂CH₂)NBn), 3.53 (2H, s, 2-CH₂N), 3.57 (2H, s, C₆H₅CH₂N), 7.25 (1H, m, C₆H₅CH₂N), 7.30 (4H, m, C₆H₅CH₂N), 7.45 (1H, ddd, H₆), 7.63 (1H, dd, H₈), 7.74 (1H, ddd, H₇) and 8.26 ppm (1H, dd, H₅); δ_{C} (CDCl₃) CH₂: 52.9, 52.9, 53.4, 53.4, 60.5, 63.0; CH: 126.7, 126.8, 127.1, 127.2, 127.2, 127.4, 129.2, 129.2, 134.8; C: 121.8, 137.9, 149.0, 153.6, 161.7.

Method 2:

2-(Chloromethyl)-3H-quinazolin-4-one (1 g, 5.14 mmoles) (prepared as described in Preparative Example 6), 1-N-benzylpiperazine (2.68 mL, 15.4 mmoles) and anhydrous potassium carbonate (0.71 g, 5.14 mmoles) were added to 200 proof ethanol (50 mL) and the mixture was heated under argon and under reflux at 80° C. for 20 h. The mixture was filtered and the solids were washed with 200 proof ethanol and the combined filtrates were evaporated to dryness. The residue was taken up in dichloromethane and washed with saturated aqueous sodium bicarbonate. The organic layer was dried (MgSO₄), filtered and evaporated to dryness. The residue was chromatographed on a silica gel column (60×5 cm) using 1.5%-3.5% (10% concentrated ammonium hydroxide in methanol)-dichloromethane as the eluant to give 2-(4-benzylpiperazin-1-ylmethyl)-3H-quinazolin-4-one (1.653 g, 96%). 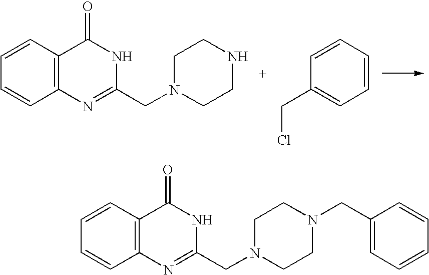
Method 3:

2-(piperazinyl-1-methyl)-3H-quinazolin-4-one (300 mg, 1.23 mmoles) (prepared as described in Preparative Example 7, Method 1, Step A above), benzyl chloride (0.424 mL, 3.69 mmoles) and anhydrous potassium carbonate (170 mg, 1.35 mmoles) were added to anhydrous acetonitrile (8 mL) and the mixture was stirred under argon at 25° C. for 22 h. The mixture was evaporated to dryness and the residue was partitioned between dichloromethane and saturated aqueous sodium bicarbonate. The organic layer was dried (MgSO₄), filtered and evaporated to dryness. The residue was chromatographed on a silica gel column (60×2.5 cm) using 3.5% (10% concentrated ammonium hydroxide in methanol)-dichloromethane as the eluant to give 2-(4-benzylpiperazin-1-ylmethyl)-3H-quinazolin-4-one (387.7 mg, 94%).

### Preparative Example 24

### N,N-DIMETHYL-N′-(2-PIPERAZIN-1-YLMETHYLQUINAZOLIN-4-YL)PROPANE-1,3-DIAMINE

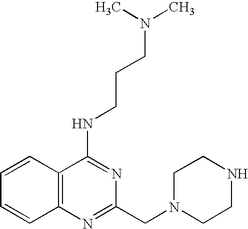

### A. 2-(4-BENZYLPIPERAZIN-1-YLMETHYL)-4-CHLOROQUINAZOLINE

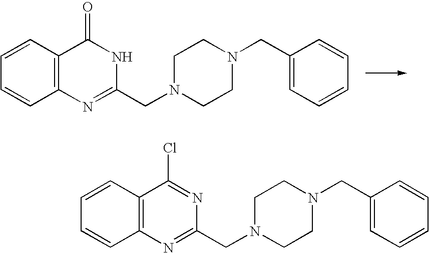

2-(4-Benzylpiperazine-1-ylmethyl)-3H-quinazolin-4-one (1.6 g, 4.78 mmoles) (prepared as described in Preparative Example 23) was dissolved in anhydrous dichloromethane (60 mL) and thionyl chloride (3.49 mL, 47.8 mmoles) was added. The mixture was stirred at 25° C. until the solid had dissolved. Anhydrous DMF (0.237 mL, 3.06 mmoles) was added and the solution was heated under argon and under reflux at 80° C. for 2.5 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (30×5 cm) using 10%-30%-40%-50% ethyl acetate in dichloromethane as the eluant to afford 2-(4-benzylpiperazin-1-ylmethyl)-4-chloroquinazoline (932.3 mg, 54%): FABMS: m/z 353.2 (MH⁺); HRFABMS: m/z 353.1532 (MH⁺). Calcd. for C₂₀H₂₂ClN₄: m/z 353.1533; δ_{H} (CDCl₃) 2.62 (4H, bs, N(CH₂CH₂)NBn), 2.75 (4H, bs, N(CH₂CH₂)NBn), 3.57 (2H, s, 2-CH₂N), 3.96 (2H, s, C₆H₅CH₂N), 7.26 (1H, m, C₆H₅CH₂N), 7.32 (4H, m, C₆H₅CH₂N), 7.67 (1H, ddd, H₆), 7.92 (1H, ddd, H₇), 8.06 (1H, dd, H₅) and 8.23 ppm (1H, dd, H₈); δ_{C} (CDCl₃) CH₂: 52.7, 52.7, 53.1, 53.1; CH: 62.9, 64.6, 125.8, 127.3, 128.3, 128.3, 128.6, 128.8, 129.5, 129.5, 134.9; C, 122.5, ˜137.0, 151.4, 162.5, 162.6.

### B. N′-[2-(4-BENZYLPIPERAZIN-1-YLMETHYL)QUINAZOLIN-4-YL]-N,N-DIMETHYLPROPANE-1,3-DIAMINE

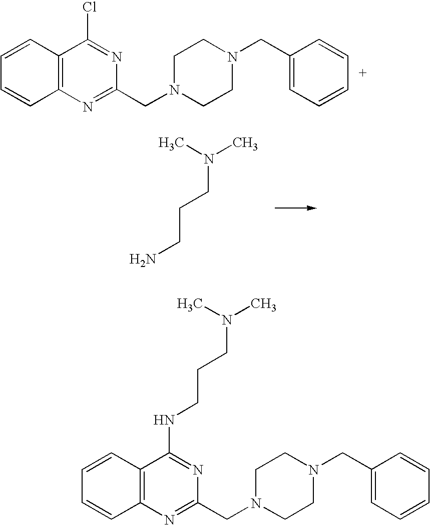

2-(4-Benzylpiperazin-1-ylmethyl)-4-chloroquinazoline (300 mg, 0.85 mmoles) (prepared as described in Preparative Example 24, Step A above) and N,N-dimethylaminopropylamine (0.214 mL, 1.7 mmoles) were dissolved in 200 proof ethanol (26.5 mL) and the mixture was heated under argon and under reflux at 80° C. for 21 h. The solution was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (60×2.5 cm) using 4%-5% (10% concentrated ammonium hydroxide in methanol)-dichloromethane as the eluant to give N′-[2-(4-benzylpiperazin-1-ylmethyl)quinazolin-4-yl]-N,N-dimethylpropane-1,3-diamine (325.7 mg, 92%): FABMS: m/z 419.1 (MH⁺); HRFABMS: m/z 419.2932 (MH⁺). Calcd. for C₂₅H₃₅N₆: m/z 419.2923; δ_{H} (CDCl₃) 1.86 (2H, m, NHCH₂CH₂CH₂N(CH₃)₂), 2.20 (6H, s, NHCH₂CH₂CH₂N(CH₃)₂), 2.56 (4H, bs, N(CH₂CH₂)NBn), 2.61 (2H, m, NHCH₂CH₂CH₂N(CH₃)₂), 2.78 (4H, bs, N(CH₂CH₂)NBn), 3.53 (2H, s, 2-CH₂N), 3.74 (2H, m, NHCH₂CH₂CH₂N(CH₃)₂), 3.76 (2H, s, C₆H₅CH₂N), 7.24 (1H, m, C₆H₅CH₂N), 7.32 (4H, m, C₆H₅CH₂N), 7.37 (1H, ddd, H₆), 7.60 (1H, dd, H₅), 7.65 (1H, ddd, H₇), 7.79 (1H, dd, H₈) and 8.63 ppm (1H, bs, NHCH₂CH₂CH₂N(CH₃)₂); δ_{C} (CDCl₃) CH₃: 45.4, 45.4; CH₂: 24.6, 42.2, 59.6, 53.1, 53.1, 53.4, 53.4, 63.2, 65.3; CH: 121.0, 125.3, 127.0, 128.2, 128.2, 128.2, 129.4, 129.4, 132.1; C: 114.1, 138.2, 149.8, 159.8, 163.3.

### C. N,N-DIMETHYL-N′-(2-PIPERAZIN-1-YLMETHYLQUINAZOLIN-4-YL)PROPANE-1,3-DIAMINE

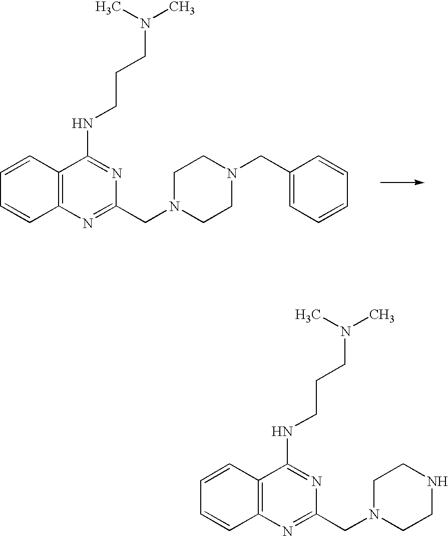

N′-[2-(4-Benzylpiperazin-1-ylmethyl)quinazolin-4-yl]-N,N-dimethylpropane-1,3-diamine (293.3 mg, 0.70 mmoles) (prepared as described in Preparative Example 24, Step B above) and ammonium formate (221 mg, 3.5 mmoles) were dissolved in methanol (16 mL). 10% Pd—C (270 mg) was added in portions under argon and the mixture was heated under argon and under reflux at 87° C. for 2 h. The catalyst was filtered off through Celite© and washed with methanol. The combined filtrates were evaporated to dryness and the residue was chromatographed on a silica gel column (30×2.5 cm) using 15% (10% concentrated ammonium hydroxide in methanol)-dichloromethane as the eluant to give N,N-dimethyl-N′-(2-piperazin-1 ylmethylquinazolin-4-yl)propane-1,3-diamine (226.2 mg, 98%): FABMS: m/z 329.1 (MH⁺); HRFABMS: m/z 329.2452 (MH⁺). Calcd. for C₁₈H₂₉N₆: m/z 329.2454; δ_{H} (CDCl₃) 1.84 (2H, m, NHCH₂CH₂CH₂N(CH₃)₂), 2.36 (6H, s, NHCH₂CH₂CH₂N(CH₃)₂), 2.58 (2H, m, NHCH₂CH₂CH₂N(CH₃)₂), 2.74 (4H, bs, N(CH₂CH₂)NH), 2.99 (4H, m, N(CH₂CH₂)NH), 3.14 (1H, bs, N(CH₂CH₂)NH), 3.74 (2H, s, 2-CH₂N), 3.74 (2H, m, NHCH₂CH₂CH₂N(CH₃)₂), 7.37 (1H, ddd, H₆), 7.56 (1H, dd, H₅), 7.64 (1H, ddd, H₇), 7.79 (1H, dd, H₈) and 8.67 ppm (1H, bs, NHCH₂CH₂CH₂N(CH₃)₂); δ_{C} (CDCl₃) CH₃: 45.6, 45.6; CH₂: 24.6, 42.6, 45.8, 45.8, 54.2, 54.2, 59.9, 65.9; CH: 120.9, 125.3, 128.3, 132.1; C: 114.1, 149.8, 159.8, 163.4.

### Preparative Example 25

### N′-[2-(2-ISOBUTYLPIPERAZIN-1-YLMETHYL)QUINAZOLIN-4-YL]-N,N-DIMETHYLPROPANE-1,3-DIAMINE

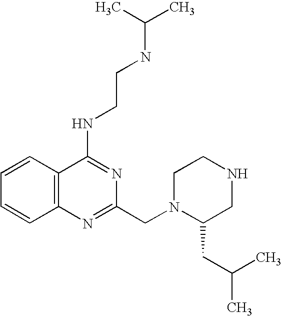

### A. [BENZYL-{2(S)-(−)-tert-BUTOXYCARBONYLAMINO-4-METHYLPENTANOYL}AMINO]ACETIC ACID ETHYL ESTER

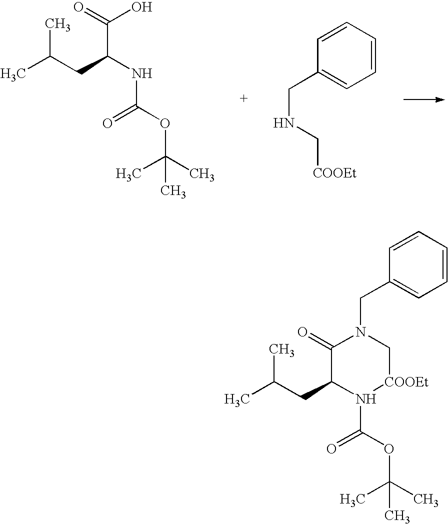

N-tert-Butoxycarbonyl-S-(−)-leucine (25 g, 112.4 mmoles), N-benzylglycine ethyl ester (20.89 g, 108.1 mmoles) and 98% 1-hydroxybenzotriazole (14.61 g, 112.4 mmoles) were dissolved in anhydrous dichloromethane (400 mL). 1M 1,3-Dicyclohexylcarbodiimide in dichloromethane (113.5 mL, 23.42 g, 118 mmoles) was added to the stirred solution at 0° C. under argon over a period of 1 h. The mixture was allowed to warm up to 25° C. over 1 h and the stirring was continued for 72 h. The solids were filtered off, rinsed with dichloromethane and the combined filtrates were evaporated to dryness. The residue was chromatograped on a silica gel column (60×5 cm) using 20% ethyl acetate in hexane as the eluant to give [benzyl-{2(S)-(−)-tert-butoxycarbonylamino -4-methyl pentanoyl}amino]acetic acid ethyl ester (40.88 g, 93%): FABMS: m/z 407.3 (MH⁺); Found: C, 65.06; H, 8.49; N, 6.81. C₂₂H₃₄N₂O₅ requires: C, 65.00, 8.43; N, 6.89; δ_{H} (CDCl₃) 0.90 (6H, d, CH(CH₃)₂), 1.25 (3H, dd, COOCH₂CH₃), 1.42 (9H, s, COOC(CH₃)₃), 4.16 (2H, m, COOCH₂CH₃) and 7.18-7.40 ppm (5H, m, CH₂C₆H₅); δ_{C} (CDCl₃) CH₃: 14.2, 21.8, 24.6, 28.4, 28.4, 28.4; CH₂: 42.0/42.7, 47.0/48.4, 50.0/50.2, 61.3/61.7; CH: 23.4/23.5, 48.6/48.7, 127.5/127.7, 128.1/128.2, 128.1/128.2, 128.7/129.0, 128.7/129.0; C: 79.6/79.8, 135.6/136.3, 155.5/155.7, 169.1, 174.1; [α]_{D}^{25° C.} −22.0° (c=1.08, MeOH).

### B. 1-BENZYL-3(S)-(+)-ISOBUTYLPIPERAZINE-2,5-DIONE

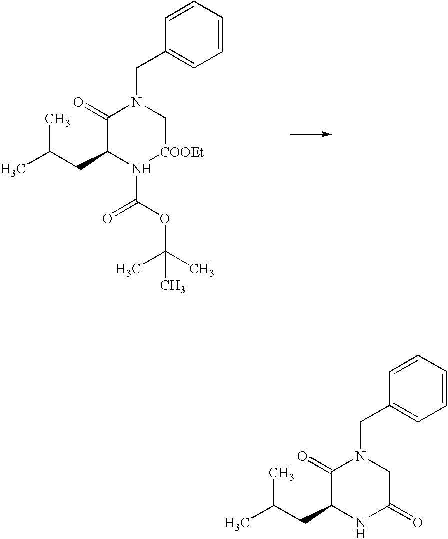

[Benzyl-{2(S)-(−)-tert-butoxycarbonylamino-4-methylpentanoyl}amino]acetic acid ethyl ester (39.83 g, 98.0 mmoles) (prepared as described in Preparative Example 25, Step A above) was dissolved in anhydrous dichloromethane (300 mL) and dry HCl gas was bubbled through the stirred solution at 25° C. for 30 min. The mixture was stirred at 25° C. for 5 h. Additional dry HCl gas was bubbled through the solution for 30 min. The mixture was then stirred at 25° C. for an additional 19 h. The mixture was evaporated to dryness and the residue was taken up in dichloromethane and washed with saturated aqueous sodium bicarbonate. The organic layer was dried (MgSO₄), filtered and evaporated to dryness to give 1-benzyl-3(S)-(+)-isobutylpiperazine-2,5-dione (25.01 g, 98%): FABMS: m/z 261.1 (MH⁺); Found: C, 69.04; H, 7.65; N, 10.72. C₁₅H₂₀N₂O₂ requires: C, 69.20; H, 7.74; N, 10.76; δ_{H} (CDCl₃) 0.96 (3H, d, CH₂CH(CH₃)₂), 0.99 (3H, d, CH₂CH(CH₃)₂), 1.64 (1H, m, CH₂CH(CH₃)₂), 1.80 (2H, m, CH₂CH(CH₃)₂), 3.79/3.88 (2H, AB system, CH₂C₆H₅), 4.06 (1H, m, 3-CH), 4.55/4.64 (2H, AB system, 6-CH₂), 6.97 (1H, bs, 4-NH), 7.26 (2H, m, CH₂C₆H₅) and 7.36 ppm (3H, m, CH₂C₆H₅); δ_{C} (CDCl₃) CH₃: 21.4, 24.3; CH₂: 43.2, 48.9, 49.8; CH: 23.2, 54.0, 129.0, 129.0, 128.2, 128.3, 128.3; C: 135.3, 166.1, 166.6; [α]_{D}^{25° C.} +13.0° (c=0.62, MeOH).

### C. 1-BENZYL-3(S)-(−)-ISOBUTYLPIPERAZINE

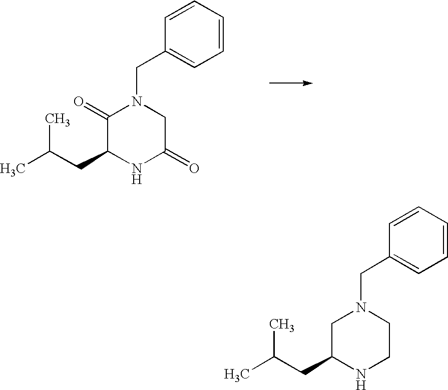

1-Benzyl-3(S)-(+)-isobutylpiperazine-2,5-dione (24.95 g, 96.0 mmoles) (prepared as described in Preparative Example 25, Step B above) was dissolved in anhydrous THF (500 mL). 1M LiAlH₄ in THF (345.0 ml, 348.7 mmoles) was added over 20 min to the stirred solution under argon at 0° C. The mixture was the heated under reflux at 65° C. for 5 h and the stirred at 25° C. for 16 h. Distilled water (100 mL) was added slowly, followed by 1N NaOH (62.5 mL). The mixture was evaporated to dryness and the residue was chromatographed on a silica gel column (45×8 cm) using 2.5%-3.0%-3.5% (10% concentrated ammonium hydroxide in methanol)-dichloromethane as the eluant to give 1-benzyl-3(S)-(−)-isobuytylpiperazine (19.06 g, 86%): FABMS: m/z 233.4 (MH⁺); HRFABMS: m/z 233.2018 (MH⁺). Calcd. for C₁₅H₂₅N₂: m/z 233.2018; δ_{H} (CDCl₃) 0.88 (3H, d, CH₂CH(CH₃)₂), 0.89 (3H, d, CH₂CH(CH₃)₂), 1.13 (1H, m, CH₂CH(CH₃)₂), 1.22 (1H, m, CH₂CH(CH₃)₂), 1.68 (2H, m, NHCHCH₂N), 2.01 (1H, m, CH₂CH(CH₃)₂), 2.72-2.87 (4H, m, NCH₂CH₂NH), 2.93 (1H, m, NHCHCH₂N), 3.45/3.53 (2H, AB system, CH₂C₆H₅), 7.27 (1H, m, CH₂C₆H₅) and 7.32 ppm (4H, m, CH₂C₆H₅); δ_{C} (CDCl₃) CH₃: 22.4, 24.3; CH₂: 43.8, 45.9, 54.0, 60.6, 63.6; CH: 23.4, 52.9, 127.1, 128.2, 128.2, 129.3, 129.3; C, 138.2; [α]_{D}^{25° C.} −5.9° (c=0.48, MeOH).

### D. 2-[4-BENZYL-2(S)-(+)-ISOBUTYLPIPERAZIN-1-YLMETHYL]-3H-QUINAZOLIN-4-ONE

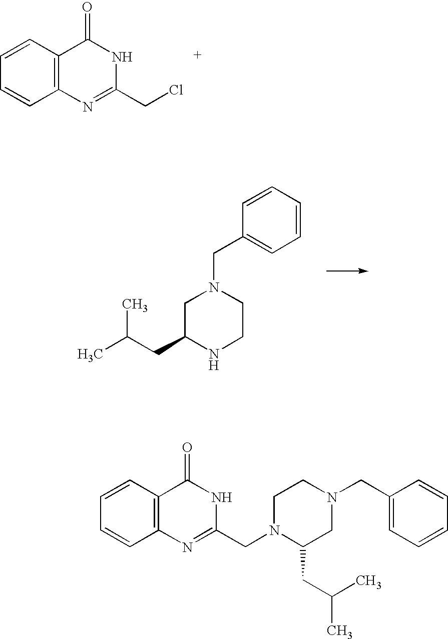

2-(Chloromethyl)-3H-quinazolin-4-one (1.675 g, 8.61 mmoles) (prepared as described in Preparative Example 6), 1-benzyl-3(S)-(−)-isobutylpiperazine (2 g, 8.61 mmoles) (prepared as described in Preparative Example 25, Step C above) and anhydrous potassium carbonate (1.176 g, 8.61 mmoles) were added to anhydrous acetonitrile (84 mL) and the mixture was heated under argon and under reflux at 80° C. for 43 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (30×5 cm) using 1% (10% concentrated ammonium hydroxide in methanol)-dichloromethane as the eluant to give 2-[4-benzyl-2(S)-(+)-isobutylpiperazin-1-ylmethyl]-3H-quinazolin-4-one (1.14 g, 34%): FABMS: m/z 391.1 (MH⁺); HRFABMS: m/z 391.2492 (MH⁺). Calcd. for C₂₄H₃₁N₄O: m/z 391.2498; δ_{H} (d₆-DMSO) 0.76 (3H, d, CH₂CH(CH₃)₂), 0.78 (3H, d, CH₂CH(CH₃)₂), 1.41 (2H, bs, CH₂CH(CH₃)₂), 2.47 (6H, s, NCH₂), 3.47/3.64 (2H, AB system, C₆H₅CH₂), 7.21 (2H, m, C₆H₅CH₂), 7.28 (3H, m, C₆H₅CH₂), 7.46/7.58 (1H, ddd, H₆), 7.55/7.71 (1H, dd, H₅), 7.77/7.96 (1H, ddd, H₇) and 8.06/8.17 ppm (1H, dd, H₈); δ_{C} (d₆-DMSO)CH₃: 21.9, 25.1; CH₂: 44.6, 52.3, 52.3, 56.4, 62.1, 62.1; CH: 23.8, 57.0, 125.9, 126.5, 127.0, 127.2/127.3, 128.2, 128.2, 128.8, 128.8, 134.5/134.9; C, 120.3/121.3, 138.4, 147.2, 159.6/161.7; [α]_{D}^{25° C.} +9.1°(c=0.56, DMSO).

### E. 2-[4-BENZYL-2(S)-(+)-ISOBUTYLPIPERAZIN-1-YLMETHYL]-4-CHLOROQUINAZOLINE

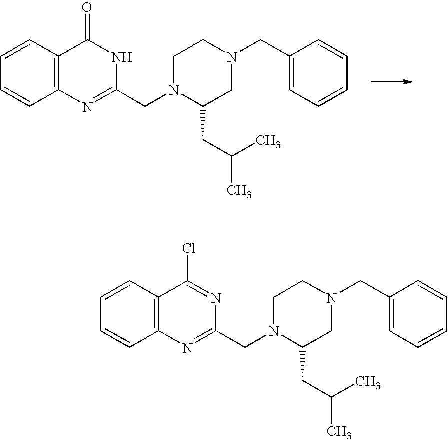

2-[4-Benzyl-2(S)-(+)-isobutylpiperazin-1-ylmethyl]-3H-quinazolin -4-one (1.04 g, 2.66 mmoles) (prepared as described in Preparative Example 25, Step D above) was dissolved in anhydrous dichloromethane (33 mL) and thionyl chloride (1.94 mL, 26.6 mmoles) was added, followed by anhydrous DMF (0.132 mL, 1.7 mmoles). The mixture was heated under argon at 80° C. for 2.5 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (30×5 cm) using 10%-20%-30%-40%-50% ethyl acetate in hexane as the eluant to give 2-[4-benzyl-2(S)-(+)-isobutylpiperazin-1-ylmethyl]-4-chloroquinazoline (174 mg, 16%): FABMS: m/z 409.1 (MH⁺); HRFABMS: m/z 409.2163 (MH⁺). Calcd. for C₂₄H₃₀ClN₄: m/z 409.2159; δ_{H} (CDCl₃) 0.84 (3H, d, CH₂CH(CH₃)₂), 0.90 (3H, d, CH₂CH(CH₃)₂), 1.44 (1H, d, CH₂CH(CH₃)₂), 1.57 (1H, d, CH₂CH(CH₃)₂) 1.88 (1H, m, CH₂CH(CH₃)₂), 2.17 (1H, dd, NCHCH₂N), 2.37 (1H, dd, NCHCH₂N), 2.62-2.84 (4H, m, NCH₂CH₂N), 3.03 (1H, m, NCHCH₂N), 3.45/3.62 (2H, AB system, C₆H₅CH₂), 4.03/4.25 (2H, AB system, 2-CH₂N), 7.24 (1H, m, C₆H₅CH₂), 7.30 (4H, m, C₆H₅CH₂), 7.67 (1H, ddd, H₆), 7.93 (1H, ddd, H₇), 8.05 (1H, dd, H₅) and 8.23 ppm (1H, dd, H₈); δ_{C} (CDCl₃) CH₃: 22.0, 24.2; CH₂: 38.6, 51.4, 52.5, 57.4, 59.6, 63.0; CH: 25.5, 57.7, 125.8, 127.1, 128.3, 128.3, 128.5, 128.7, 129.3, 129.3, 134.8; C: 122.4, 137.9, 151.3, 162.4, 163.2; [α]_{D}^{25° C.} +47.4° (c=0.33, MeOH).

### F. N′-[2-{4-BENZYL-2(S)-(+)-ISOBUTYLPIPERAZIN-1-YLMETHYL}QUINAZOLIN-4-YL]-N,N-DIMETHYLPROPANE-1,3-DIAMINE

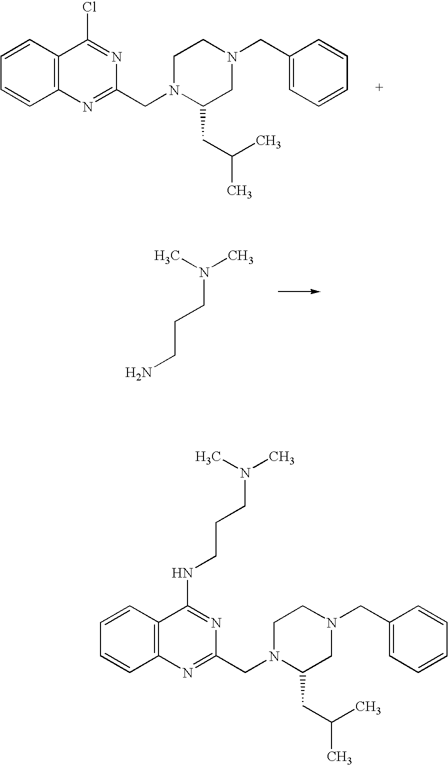

2-[4-Benzyl-2(S)-(+)-isobutylpiperazin-1-ylmethyl]-4-chloroquinazoline (164.3 mg, 0.402 mmoles) (prepared as described in Preparative Example 25, Step E above) and N,N-dimethylaminopropylamine (0.101 mL, 0.804 mmoles) were dissolved in anhydrous acetonitrile (12 mL) and the mixture was stirred under argon at 25° C. for 67 h. The mixture was evaporated to dryness and the residue was chromatographed on a silica gel column (30×2.5 cm) using 5% (10% concentrated ammonium hydroxide in methanol)-dichloromethane as the eluant to give N′-[2-{4-Benzyl-2(S)-(+)-isobutylpiperazin -1-ylmethyl}quinazolin-4-yl]-N,N-dimethylpropane-1,3-diamine (144.8 mg, 76%): FABMS: m/z 475.4 (MH⁺); HRFABMS: m/z 475.3543 (MH⁺). Calcd. for C₂₉H₄₃N₆: m/z 475.3549; δ_{H} (CDCl₃) 0.82 (3H, d, CH₂CH(CH₃)₂), 0.91 (3H, d, CH₂CH(CH₃)₂), 1.40 (1H, d, CH2CH(CH₃)₂), 1.57 (1H, d, CH₂CH(CH₃)₂), 1.83 (3H, overlapping multiplets, CH₂CH(CH₃)₂ and NHCH₂CH₂CH₂N(CH₃)₂), 2.09 (1H, dd, NCHCH₂N), 2.34 (1H, dd, NCHCH₂N), 2.37 (6H, s, N(CH₃)₂), 2.54-2.69 (3H, overlapping multiplets, NCH₂CH₂N and NHCH₂CH₂CH₂N(CH₃)₂), 2.76-2.89 (3H, overlapping multiplets, NCH₂CH₂N), 3.07 (1H, m, NCHCH₂N), 3.42/3.58 (2H, AB system, C₆H₅CH₂), 3.74 (2H, m, NHCH₂CH₂CH₂N(CH₃)₂), 3.87/3.98 (2H, AB system, 2-CH₂N), 7.24 (2H, m, C₆H₅CH₂), 7.30 (3H, m, C₆H₅CH₂), 7.37 (1H, ddd, H₆), 7.54 (1H, dd, H₅), 7.64 (1H, ddd, H₇), 7.77 (1H, dd, H₈) and 8.55 ppm (1H, bs, NHCH₂CH₂CH₂N(CH₃)₂); δ_{C} (CDCl₃) CH₃: 22.0, 24.3, 45.6, 45.6; CH₂: 24.8, 38.8, 42.6, 51.7, 52.9, 58.2, 60.0, 60.2, 60.2, 63.2; CH: 25.4, 57.2, 120.9, 125.1, 126.9, 128.2, 128.2, 128.3, 129.2, 129.2, 132.0; C: 114.0, 138.4, 149.9, 159.7, 164.2; [α]_{D}^{25° C.} +44.4° (c=0.40, MeOH).

### G. N′-[2-{2(S)-(+)-ISOBUTYLPIPERAZIN-1-YLMETHYL}QUINAZOLIN-4-YL]-N,N-DIMETHYLPROPANE-1,3-DIAMINE

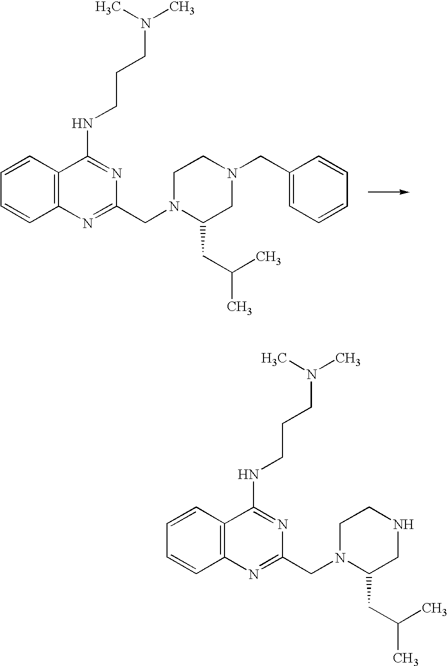

N′-[2-{4-Benzyl-2(S)-(+)-isobutylpiperazin-1-ylmethyl}quinazolin-4-yl]-N,N-dimethylpropane-1,3-diamine (137.8 mg, 0.29 mmoles) (prepared as described in Preparative Example 25, Step F above) and ammonium formate (91.8 mg, 1.45 mmoles) were dissolved in methanol (7 mL) and 10% Pd—C (112 mg) was added under argon. The mixture was heated under argon at 87° C. for 1.75 h. After 16 h at 25° C. additional 10% Pd—C (50 mg) was added and the mixture was heated at 87° C. for an additional 2 h. The catalyst was filtered off through Celite© and washed with methanol. The combined filtrates were evaporated to dryness and the residue was chromatographed on a silica gel column (15×2.5 cm) using 5% (10% concentrated ammonium hydroxide in methanol)-dichloromethane as the eluant to give unreacted N′-[2-{4-Benzyl-2-isobutylpiperazin-1-ylmethyl}quinazolin-4-yl]-N,N-dimethylpropane-1,3-diamine (36.5 mg, 27%) and N′-[2-{2(S)-(+)-isobutylpiperazin-1-ylmethyl}quinazolin-4-yl]-N,N-dimethylaminopropane-1,3-diamine (24.7 mg, 22%): FABMS: m/z 385.4 (MH⁺); HRFABMS: m/z 385.3076 (MH⁺). Calcd. for C₂₂H₃₇N₆: m/z 385.3080; Found: C, 77.22; H, 10.18; N, 11.84; C₁₅H₂₄N₂ requires: C, 77.53; H, 10.41; N, 12.06; δ_{H} (CDCl₃) 0.82 (3H, d, CH₂CH(CH₃)₂), 0.93 (3H, d, CH₂CH(CH₃)₂), 1.33 (2H, d, CH₂CH(CH₃)₂), 1.63 (1H, m, NHCH₂CH₂CH₂N(CH₃)₂), 1.84 (3H, overlapping multiplets, CH₂CH(CH₃)₂ and NHCH₂CH₂CH₂N(CH₃)₂), 2.09 (1H, dd, NCHCH₂N), 2.37 (1H, dd, NCHCH₂N), 2.37 (6H, s, N(CH₃)₂), 2.58 (3H, overlapping multiplet, NHCH₂CH₂CH₂N(CH₃)₂ and NCHCH₂N), 2.82/2.93 (4H, overlapping multiplets, NCH₂CH₂N), 3.08 (1H, m, NCHCH₂N), 3.74 (2H, m, NHCH₂CH₂CH₂N(CH₃)₂), 3.88/3.98 (2H, AB system, 2-CH₂N), 7.36 (1H, ddd, H₆), 7.56 (1H, dd, H₅), 7.64 (1H, ddd, H₇), 7.77 (1H, dd, H₈) and 8.62 (1H, bs, NHCH₂CH₂CH₂N(CH₃)₂); δ_{C} (CDCl₃) CH₃: 22.0, 24.3, 45.5, 45.5; CH₂: 24.7, 38.6, 42.7, 45.7, 50.4, 52.3, 59.9, 60.2; CH: 25.3, 57.6, 121.0, 125.2, 128.2, 132.0; C: 114.0, 149.8, 159.7, 164.2; [α]_{D}^{25° C.} +30.6° (c=0.31, MeOH).

### Preparative Example 26

### DIMETHYL-[3-(2-PIPERAZIN-1-YLMETHYLQUINAZOLIN-4-YLOXY)PROPYL]AMINE

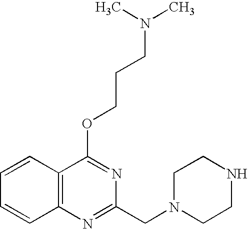

### A. {3-[2-(4-BENZYLPIPERAZIN-1-YLMETHYL)QUINAZOLIN-4-YLOXY]PROPYL}-DIMETHYLAMINE

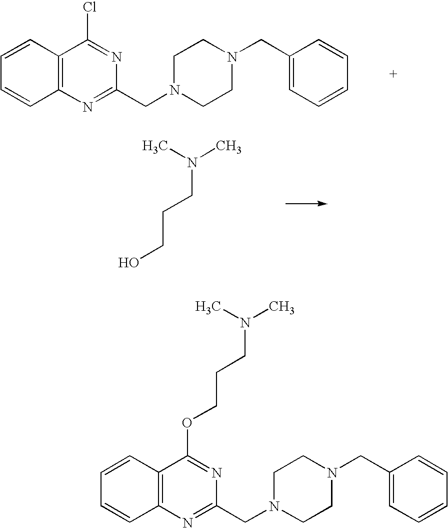

2-(4-Benzylpiperazin-1-ylmethyl)-4-chloroquinazoline (550 mg, 1.56 mmoles) (prepared as described in Preparative Example 24, Step A), 3-dimethylaminopropanol (0.369 mL, 3.12 mmoles) and anhydrous potassium carbonate (215.4 mg, 1.56 mmoles) were added to anhydrous acetonitrile (25 mL) and the mixture was heated under argon at 80° C. for 17 h. The solids were filtered off and rinsed with acetonitrile and dichloromethane and the combined filtrates were evaporated to dryness. The residue was chromatographed on a silica gel column (60×2.5 cm) using 2%-5% (10% concentrated ammonium hydroxide in methanol)-dichloromethane as the eluant to give {3-[2-(4-benzylpiperazin-1-ylmethyl)quinazolin-4-yloxy]propyl}dimethylamine (324.9 mg, 50%): FABMS: m/z 420.0 (MH⁺); HRFABMS: m/z 420.2760 (MH⁺); Calcd. for C₂₅H₃₄N₅: m/z 420.2763; δ_{H} (CDCl₃) 2.04 (2H, m, OCH₂CH₂CH₂N(CH₃)₂), 2.27 (6H, s, OCH₂CH₂CH₂N(CH₃)₂), 2.50 (2H, dd, OCH₂CH₂CH₂N(CH₃)₂), 2.56 (4H, bs, NCH₂CH₂N), 2.74 (4H, bs, NCH₂CH₂N), 3.52 (2H, s, CH₂C₆H₅), 3.84 (2H, s, 3-CH₂N), 4.62 (2H, dd, OCH₂CH₂CH₂N(CH₃)₂), 7.23 (2H, m, CH₂C₆H₅), 7.28 (3H, m, CH₂C₆H₅), 7.49 (1H, ddd, H₆), 7.77 (1H, ddd, H₇), 7.92 (1H, dd, H₄) and 8.12 ppm (1H, dd, H₈); δ_{C} (CDCl₃) CH₃: 45.6, 45.6; CH₂: 27.2, 53.1, 53.1, 53.4, 53.4, 56.5, 63.2, 65.1, 65.4; CH: 123.3, 126.4, 127.0, 127.7, 128.2, 128.2, 129.3, 129.3, 133.2; C: 115.2, 138.2, 151.3, 162.8, 166.7 and 2-(4-benzylpiperazine-1-ylmethyl)-3H-quinazolin-4-one (94.5 mg, 18%).

### B. DIMETHYL-[3-(2-PIPERAZIN-1-YLMETHYLQUINAZOLIN-4-YLOXY)PROPYL]-AMINE

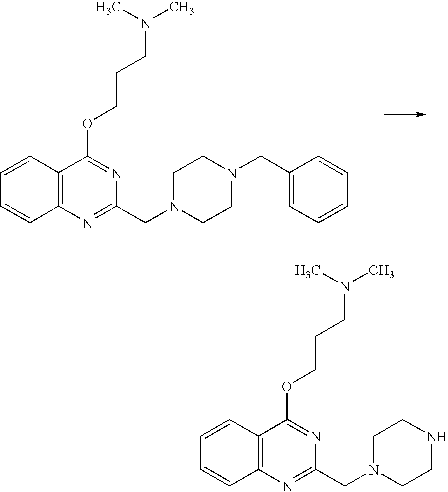

{3-[2-(4-Benzylpiperazin-1-ylmethyl)quinazolin-4-yloxy]propyl}dimethylamine (269.5 mg, 0.64 mmoles) (prepared as described in Preparative Example 26, Step A above) and ammonium formate (203 mg, 3.21 mmoles) were dissolved in methanol (15 mL) and 10% Pd—C (248 mg) was added under argon. The mixture was heated under argon at 87° C. for 1 h. The catalyst was filtered off through Celite© and the latter was rinsed with methanol. The combined filtrates were evaporated to dryness and the residue was chromatographed on a silica gel column (60×2.5 cm) using 10% (10% concentrated ammonium hydroxide in methanol)-dichloromethane as the eluant to give dimethyl-[3-(2-piperazin-1-ylmethylquinazolin-4-yloxy)propyl]amine (148.4 mg, 70%): FABMS: m/z 330.2 (MH⁺); HRFABMS: m/z 330.229 (MH⁺). Calcd. for C₁₈H₂₈N₅O: m/z 330.2294; δ_{H} (CDCl₃) 2.04 (2H, m, OCH₂CH₂CH₂N(CH₃)₂), 2.26 (6H, s, OCH₂CH₂CH₂N(CH₃)₂), 2.32 (1H, bs, NH), 2.48 (2H, dd, OCH₂CH₂CH₂N(CH₃)₂), 2.67 (4H, bs, NCH₂CH₂N), 2.96 (4H, m, NCH₂CH₂N), 3.82 (2H, s, 3-CH₂N), 4.62 (2H, dd, OCH₂CH₂CH₂N(CH₃)₂), 7.49 (1H, ddd, H₆), 7.77 (1H, ddd, H₇), 7.92 (1H, dd, H₄) and 8.11 ppm (1H, dd, H₈); δ_{C} (CDCl₃) CH₃: 45.6, 45.6; CH₂: 27.2, 46.0, 46.0, 54.6, 54.6, 56.5, 65.4, 65.7; CH: 123.4, 126.5, 127.7, 133.4; C: 115.2, 151.3, 162.7, 166.7.

### Preparative Example 27

### 2-AMINO-N-METHOXY-3-METHYLBUTYRAMIDE

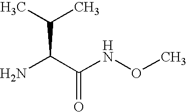

### A. [1(S)-(−)-METHOXYCARBAMOYL-2-METHYLPROPYL]CARBAMIC ACID tert-BUTYL ESTER

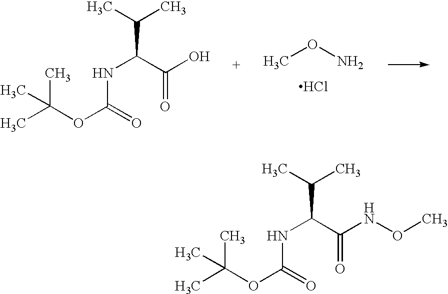

N-(tert-Butoxycarbonyl)-L(−)-valine (1 g, 4.58 mmoles), methoxy]amine hydrochloride (499.7 mg, 5.98 mmoles), 1-[3-(dimethylamino)propyl]-3-ethylcarbodiimide hydrochloride (1.15 g, 5.98 mmoles), hydroxybenzotriazole (808.5 mg, 5.98 mmoles) and N-methylmorpholine (1.21 g, 1.316 mL, 11.91 mmoles) were dissolved in anhydrous DMF (20 mL) and the mixture was stirred at 25° C. for 89 h. The solution was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (60×2.5 cm) using 0.3%-3% (10% concentrated ammonium hydroxide in methanol)-dichloromethane as the eluant to give [1(S)-(−)-methoxycarbamoyl -2-methylpropyl]carbamic acid tert-butyl ester (857.7 mg, 76%): FABMS: m/z 247.4 (MH⁺); Found: C, 54.03; H, 9.18; N, 11.38; C₁₁H₂₂N₂O₄ requires: C, 53.64; H, 9.00; N, 11.37; δ_{H} (CDCl₃) 0.96 (6H, d, CHCH(CH₃)₂), 1.43 (9H, s, NHCOOC(CH₃)₃), 2.05 (1H, dq, CHCH(CH₃)₂), 3.76 (1H, bs, NH), 3.76 (3H, s, CONHOCH₃), 5.23 (1H, m, CHCH(CH₃)₂) and 9.61 ppm (1H, bs, NH); δ_{C} (CDCl₃) CH₃: 18.5, 19.2, 28.4, 28.4, 28.4, 57.8; CH: 30.8, 64.3; C: 80.4, 156.1, 165.2, 169.2; [α]]_{D}^{25° C.} −32.7° (c=1.02, MeOH).

### B. 2(S)-(+)-AMINO-N-METHOXY-3-METHYLBUTYRAMIDE

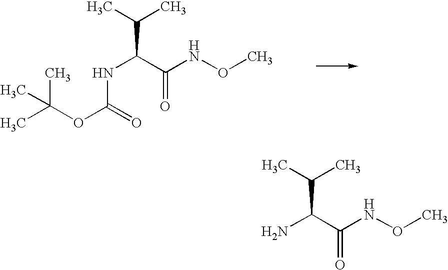

[1(S)-(−)-Methoxycarbamoyl-2-methyl propyl]carbamic acid tert-butyl ester (812 mg, 3.3 mmoles) (prepared as described in Preparative Example 27, Step A above) was dissolved in methanol (10 mL) and 10% concentrated sulfuric acid in 1,4-dioxane (v/v) (10 mL) was added. The mixture was stirred at 25° C. for 4 h. The reaction was diluted with methanol and BioRad® AG1X8 (OH⁻) resin was added until the pH reached 10. The resin was filtered off and washed with methanol. The combined filtrates were evaporated to dryness the residue was chromatographed on a silica gel column (30×2.5 cm) using 5% (10% concentrated ammonium hydroxide in methanol)-dichloromethane as the eluant to give (S)-(+)-amino-N-methoxy-3-methylbutyramide (172.7 mg, 48%): FABMS: m/z 147.2 (MH⁺); Found: C, 49.04; H, 9.39; N, 18.65; C₆H₁₄N₂O₂ requires; C, 49.30; H, 9.65; N, 19.16; δ_{H} (CDCl₃) 0.87 (6H, d, CHCH(CH₃)₂), 0.98 (9H, s, NHCOOC(CH₃)₃), 1.40 (2H, bs, NH₂), 2.29 (1H, dq, CHCH(CH₃)₂), 3.25 (1H, d, CHCH(CH₃)₂) and 3.78 (3H, s, CONHOCH₃); δ_{C} (CDCl₃) CH₃: 16.2, 19.4, 64.5; CH: 31.0, 59.5; C, 171.5; [α]_{D}^{25° C.} +39.5° (c=0.53, MeOH).

### Preparative Example 28

### 2(S)-(+)-AMINO-N-ETHOXY-3-METHYLBUTYRAMIDE

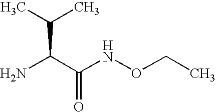

### A. (1-ETHOXYCARCARBAMOYL-2(S)-(−)-METHYLPROPYL)CARBAMIC ACID tert-BUTYL ESTER

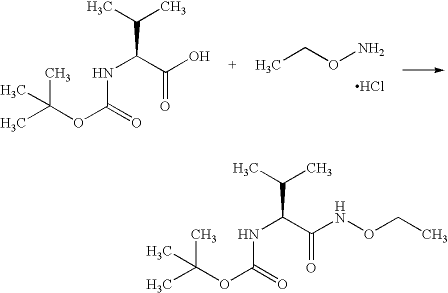

N-(tert-Butoxycarbonyl)-L(−)-valine (1 g, 4.58 mmoles), ethoxy]amine hydrochloride (583.7 mg, 5.98 mmoles), 1-[3-(dimethylamino)propyl]-3-ethylcarbodiimide hydrochloride (1.15 g, 5.98 mmoles), hydroxybenzotriazole (808.5 mg, 5.98 mmoles) and N-methylmorpholine (1.21 g, 1.316 mL, 11.91 mmoles) were dissolved in anhydrous DMF (20 mL) and the mixture was stirred at 25° C. for 89 h. The solution was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (60×2.5 cm) using 0.3%-3% (10% concentrated ammonium hydroxide in methanol)-dichloromethane as the eluant to give [1(S)-(−)-ethoxycarbamoyl -2-methylpropyl]carbamic acid tert-butyl ester (934.1 mg, 78%): FABMS: m/z 261.3 (MH⁺); Found: C, 55.83; H, 9.28; N, 10.78; C₁₂H₂₄N₂O₄ requires: C, 55.36; H, 9.29; N, 10.76; δ_{H} (CDCl₃) 0.96 (6H, d, CHCH(CH₃)₂), 1.27 (3H, t, OCH₂CH₃), 1.43 (9H, s, NHCOOC(CH₃)₃), 2.06 (1H, dq, CHCH(CH₃)₂), 3.73 (1H, t, NH), 3.95 (2H, q, CONHOCH₂CH₃), 5.18 (1H, d, CHCH(CH₃)₂) and 9.32 ppm (1H, bs, NH); δ_{C} (CDCl₃) CH₃: 13.5, 18.5, 19.2, 28.4, 28.4, 28.4; CH₂: 72.2; CH: 30.7, 57.9; C: 80.3, 156.1, 169.2; [α]_{D}^{25° C.} −35.9° (c=1.05, MeOH).

### B. 2(S)-(+)-AMINO-N-ETHOXY-3-METHYLBUTYRAMIDE

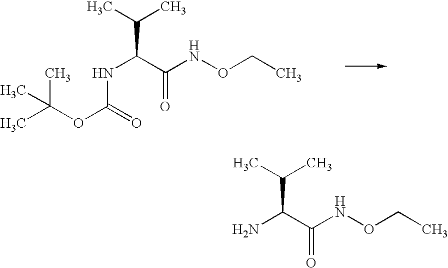

[1(S)-(−)-Ethoxycarbamoyl-2-methylpropyl]carbamic acid tert-butyl ester (894 mg, 3.4 mmoles) (prepared as described in Preparative Example 28, Step A above) was dissolved in methanol (10 mL) and 10% concentrated sulfuric acid in 1,4-dioxane (v/v) (10 mL) was added. The mixture was stirred at 25° C. for 4 h. The reaction was diluted with methanol and BioRad®AG1X8 (OH⁻) resin was added until the pH reached 10 The resin was filtered off and washed with methanol. The combined filtrates were evaporated to dryness the residue was chromatographed on a silica gel column (30×2.5 cm) using 10% (10% concentrated ammonium hydroxide in methanol)-dichloromethane as the eluant to give (S)-(+)-amino-N-ethoxy-3-methylbutyramide (352 mg, 64%): FABMS: m/z 161.3 (MH⁺); Found: C, 52.75; H, 9.84; N, 17.33; C₇H₁₆N₂O₂ requires; C, 52.48; H, 10.07; N, 17.49; δ_{H} (CDCl₃) 0.87 (6H, d, CHCH(CH₃)₂), 0.97 (9H, s, NHCOOC(CH₃)₃), 1.27 (3H, t, CONHOCH₂CH₃), 1.36 (2H, bs, NH₂), 2.26 (1H, dq, CHCH(CH₃)₂), 3.24 (1H, d, CHCH(CH₃)₂), 3.97 (2H, q, CONHOCH₂CH₃) and 9.57 ppm (1H, bs, NH); δ_{C} (CDCl₃) CH₃: 13.5, 16.3, 19.4, 64.5; CH₂: 72.2; CH: 31.0, 59.5; C, 171.6; [α]_{D}^{25° C.} +33.9° (c=0.51, MeOH).

### Preparative Example 29

### 2(S)-(−)-tert-BUTOXYCARBONYLAMINO-4-DIMETHYLAMINOBUTYRIC ACID

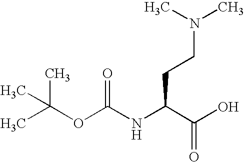

### A. 4-BENZYLOXYCARBONYLAMINO-2(S)-(−)-tert-BUTOXYCARBONYLAMINOBUTYRIC ACID

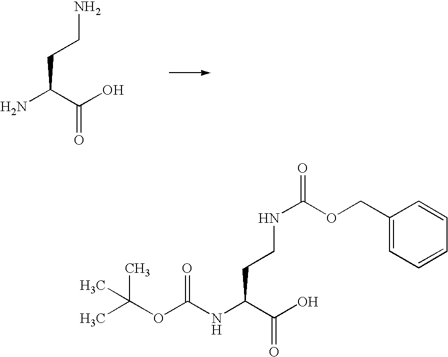

Using the procedure described in the literature for the conversion of racemic 2,4-diaminobutyric acid into racemic 4-benzyloxycarbonylamino-2-tert-butoxycarbonylaminobutyric acid [A. D. Borthwick, S. J. Angier, A. J. Crame, A. M. Exall, T. M. Haley, G. J. Hart, A. M. Mason, A. M. K. Pennell and G. G. Weingarten, J. Med. Chem., 43(23), 4452-4464 (2000)], 2(S)-(−)-2,4-diaminobutyric acid (20.78 g, 108 mmoles) was converted into 4-benzyloxycarbonylamino-2(S)-(−)-tert-butoxycarbonylaminobutyric acid (17.07 g, 69%): FABMS: m/z 353.0 (MH⁺); δ_{H} (CDCl₃) 1.43 (9H, s, COOC(CH₃)₃), 5.04/5.13 (2H, AB system, CH₂C₆H₅) and 7.37 ppm (5H, m, CH₂C₆H₅); δ_{C} (CDCl₃) CH₃: 28.4, 28.4, 28.4; CH₂: 33.4, 37.2, 67.1; CH: 50.9, 128.2, 128.2, 128.6, 128.6, 128.6; C: 80.5, 136.4, 156.0, 157.1, 176.0; [α]_{D}^{25° C.} −13.5° (c=0.51, MeOH). The (S)-(−)-Isomer has also been prepared by an alternative procedure [K. Vogler, R. O. Studer, P. Lanz, W. Lergier and E. Bohni, Helv. Chim. Acta, 48(5), 1161-1177 (1965)].

### B. 2(S)-(−)-tert-BUTOXYCARBONYLAMINO-4-DIMETHYLAMINOBUTYRIC ACID

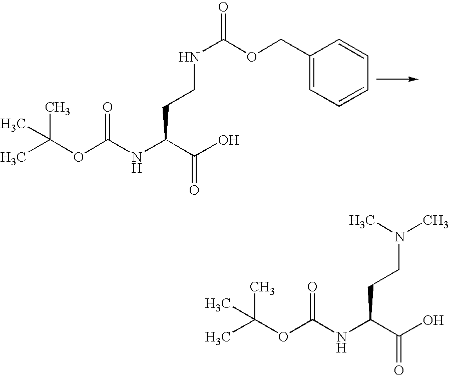

4-Benzyloxycarbonylamino-2(S)-(−)-tert-butoxycarbonylaminobutyric acid (17 g, 48.2 mmoles) and 37% aqueous formaldehyde (9.03 mL, 115.8 mmoles) were dissolved in methanol-distilled water (1:1) (260 mL). 10% Pd—C (wet; ˜7 g) was added under argon and the mixture was hydrogenated at 25° C. and 50 psi on a Parr hydrogenator for 74 h. The catalyst was filtered off through Celite© and the latter was washed with methanol-distilled water (1:1). The combined filtrates were evaporated to dryness to give 2(S)-(−)-tert-butoxycarbonylamino-4-dimethylaminobutyric acid (10.89 g, 92%): FABMS: m/z 247.0 (MH⁺); HRFABMS: m/z 247.1660 (MH⁺). Calcd. for C₁₁H₂₃N₂O₄: m/z 247.1658; δ_{H} (CDCl₃) 1.34 (9H, s, COOC(CH₃)₃), 1.80 (1H, m, CHCH₂CH₂N(CH₃)₂), 1.87 (1H, m, CHCH₂CH₂N(CH₃)₂), 2.43 (6H, s, N(CH₃)₂), 2.68 (1H, m, CHCH₂CH₂N(CH₃)₂), 2.79 (1H, m, CHCH₂CH₂N(CH₃)₂), 3.74 (1H, m, CHCH₂CH₂N(CH₃)₂) and 6.47 ppm (1H, d, NH); δ_{C} (CDCl₃) CH₃: 28.3, 28.3, 28.3, 42.7, 42.7; CH₂: 28.3, 56.2; CH: 53.9; C: 79.5, 155.8, 175.2; [α]_{D}^{25° C.} −1.7° (c=0.30, MeOH).

### Preparative Example 30

### 2(S)-tert-BUTOXYCARBONYLAMINO-4-DIMETHYLAMINOBUTYRIC ACID ISOBUTYL ESTER

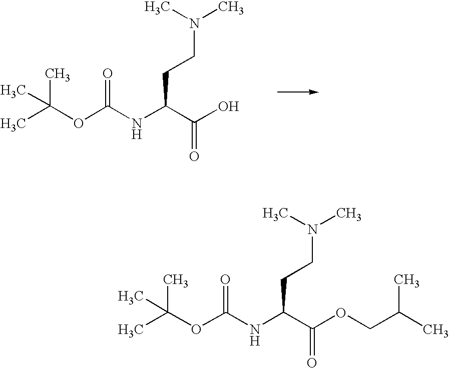

2(S)-(−)-tert-Butoxycarbonylamino-4-dimethylaminobutyric acid (5 g, 20.3 mmoles) (prepared as described in Preparative Example 29, step B above), N-methylmorpholine (2.26 g, 2.46 mL, 22.3 mmoles) and isobutyl chloroformate (3.05 g, 2.9 mL, 22.3 mmoles) were dissolved in anhydrous THF (200 mL) and the mixture was stirred at 0° C. for 1.5 h. Conc. ammonium hydroxide (30%) (10 mL) was added and the mixture was stirred at 0° C. for 3 h. The mixture was evaporated to dryness and the product was chromatographed on a silica gel column (30×5 cm) using 1% (10% concentrated ammonium hydroxide in methanol)-dichloromethane as the eluant to give 2(S)-tert-butoxycarbonylamino-4-dimethylaminobutyric acid isobutyl ester (3.56 g, 58%): FABMS: m/z 303.1 (MH⁺); HRFABMS: m/z 303.2287 (MH⁺). Calcd. for C₁₅H₃₁N₂O₄: m/z 303.2284; δ_{H} (CDCl₃) 0.93 (6H, d, COOCH₂CH(CH₃)₂), 1.42 (9H, s, COOC(CH₃)₃), 1.83 (1H, m, OCH₂CH(CH₃)₂), 1.92 (1H, m, CHCH₂CH₂N(CH₃)₂), 1.97 (1H, m, CHCH₂CH₂N(CH₃)₂), 2.22 (6H, s, N(CH₃)₂), 2.31 (1H, m, CHCH₂CH₂N(CH₃)₂), 2.40 (1H, m, CHCH₂CH₂N(CH₃)₂), 3.38 (2H, m, OCH₂CH(CH₃)₂), 4.33 (1H, m, CHCH₂CH₂N(CH₃)₂) and 5.90 ppm (1H, m, NH); δ_{C} (CDCl₃) 19.1, 19.1, 28.4, 28.4, 28.4, 45.4, 45.4; CH₂: 29.5, 56.0, 71.3; CH: 27.8, 53.0; C: 79.6, 155.7, 172.8; [α]_{D}^{25° C.} 0° (c=0.53, MeOH).

### Preparative Example 31

### 2(S)-(+)-AMINO-4-DIMETHYLAMINOBUTYRIC ACID ISOBUTYL ESTER

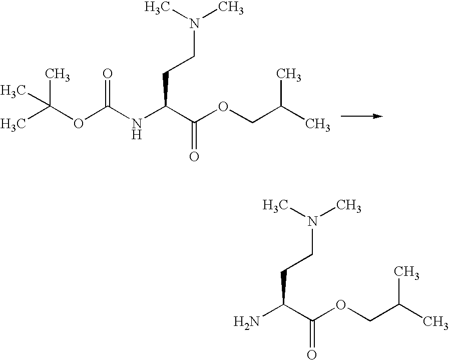

2(S)-tert-Butoxycarbonylamino-4-dimethylaminobutyric acid isobutyl ester (1.6 g, 5.3 mmoles) (prepared as described in Preparative Example 30 above) was dissolved in anhydrous dichloromethane (100 mL) and the solution was cooled to 0° C. under nitrogen with stirring. Tin (II) triflate (2.21 g, 5.3 mmoles) was added in portions to the stirred solution at 0° C. The mixture was then stirred at 25° C. for 48 h. A viscous gum separated that eventually solidified. The reaction mixture was partitioned between dichloromethane and saturated aqueous sodium bicarbonate. The aqueous layer was extracted twice with dichloromethane (200 mL) and the combined extracts were dried (MgSO₄), filtered and evaporated to dryness. The residue was chromatographed on a silica gel column (30×5 cm) using 3% (10% concentrated ammonium hydroxide in methanol)-dichloromethane as the eluant to give unreacted 2(S)-tert-butoxycarbonylamino-4-dimethylaminobutyric acid isobutyl ester (410.5 mg, 26%) and 2(S)-(+)-amino-4-dimethylaminobutyric acid isobutyl ester (77.1 mg, 7%).

The unreacted 2(S)-tert-butoxycarbonylamino-4-dimethylaminobutyric acid isobutyl ester (410.5 mg) was taken up in 10% (v/v) concentrated sulfuric acid in dioxane (5 mL) and the mixture was stirred at 25° C. for 2 h. BioRad AG1X8 (OH⁻) resin was added until the pH reached 8 and the resin was then filtered off and washed with methanol. The filtrate was evaporated to dryness and the residue was chromatographed on a silica gel column (30×1.5 cm) using 5% (10% concentrated ammonium hydroxide in methanol)-dichloromethane as the eluant to give 2(S)-(+)-amino-4-dimethylaminobutyric acid isobutyl ester (166.3 mg, 16%) (Total yield: 243.4 mg, 23%): LCMS: m/z 203.1 (MH⁺); HRFABMS: m/z 203.1756 (MH⁺). Calcd. For C₁₀H₂₃N₂O₂: m/z 203.1760; δ_{H} (CDCl₃) 0.99 (6H, d, COOCH₂CH(CH₃)₂), 1.74 (1H, m, COOCH₂CH(CH₃)₂), 1.74 (2H, m, CHCH₂CH₂N(CH₃)₂), 2.00 (2H, m, NH₂), 2.27 (6H, s, N(CH₃)₂), 2.41 (1H, m, CHCH₂CH₂N(CH₃)₂), 2.47 (1H, m, CHCH₂CH₂N(CH₃)₂), 3.58 (1H, m, CHCH₂CH₂N(CH₃)₂) and 3.97 ppm (2H, m, COOCH₂CH(CH₃)₂); δ_{C} (CDCl₃) CH₃: 19.5, 19.5, 45.9, 45.9; CH₂: 32.9, 56.7, 71.4; CH: 28.2, 53.6; C, 176.6; [α]_{D}^{25° C.} +2.90° (c=1.00, MeOH).

### Preparative Example 32

### 2(S)-tert-BUTOXYCARBONYLAMINO-4-DIMETHYLAMINOBUTYRIC ACID METHYL ESTER

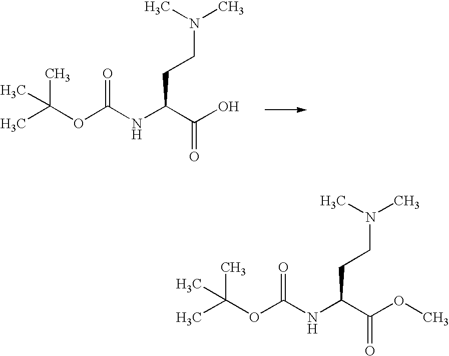

2(S)-(−)-tert-Butoxycarbonylamino-4-dimethylaminobutyric acid (prepared as described in Preparative Example 29, Step B above) may be reacted with either diazomethane, or trimethylsilyl diazomethane in a suitable inert solvent such as THF using methods well known to those skilled in the art, to give 2(S)-tert-butoxycarbonylamino -4-dimethylaminobutyric acid methyl ester.

### Preparative Example 33

### 2(S)-AMINO-4-DIMETHYLAMINOBUTYRIC ACID METHYL ESTER

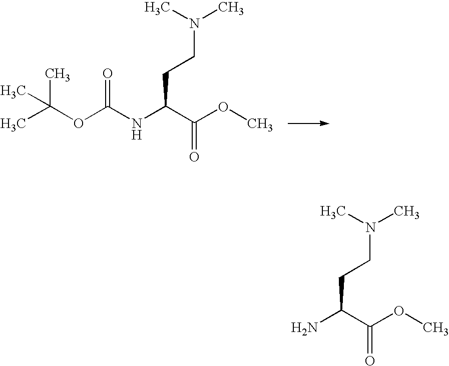

2(S)-tert-Butoxycarbonylamino-4-dimethylaminobutyric acid methyl ester (prepared as described in Preparative Example 32 above) may be deprotected as described in Preparative Example 27, Step B to give 2(S)-amino-4-dimethylaminobutyric acid methyl ester.

### Preparative Example 34

### 2(S)-tert-BUTOXYCARBONYLAMINO-4-DIMETHYLAMINOBUTYRAMIDE

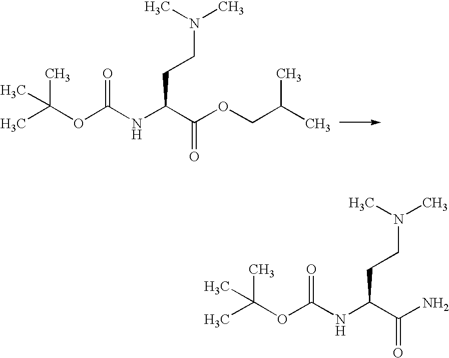

2(S)-tert-Butoxycarbonylamino-4-dimethylaminobutyric acid isobutyl ester (1.5 g, 0.5 mmoles) (prepared as described in Preparative Example 30 above) was dissolved in anhydrous methanol (14 mL) and the solution was stirred and cooled to 0° C. and then saturated with anhydrous ammonia for 15 min. The vessel was sealed and stirred at 25° C. for 243 h. The reaction mixture was evaporated to dryness and the residue was chromatographed on a silica gel column (30×5 cm) using 10% (10% concentrated ammonium hydroxide in methanol)-dichloromethane as the eluant to give 2(S)-tert-butoxycarbonylamino-4-dimethylaminobutyramide (599.5 mg, 49%): LCMS: m/z 246.1 (MH⁺); HRFABMS: m/z 246.1827 (MH⁺). Calcd. for C₁₁H₂₄N₃O₃: m/z 246.1818; δ_{H} (CDCl₃) 1.45 (9H, s, COOC(CH₃)₃), 1.84 (1H, m, CHCH₂CH₂N(CH₃)₂), 1.96 (1H, m, CHCH₂CH₂N(CH₃)₂), 2.23 (6H, s, N(CH₃)₂), 2.40 (1H, m, CHCH₂CH₂N(CH₃)₂), 2.48 (1H, m, CHCH₂CH₂N(CH₃)₂), 4.23 (1H, m, CHCH₂CH₂N(CH₃)₂), 5.23 (1H, m, NH), 6.30 (1H m, CONH₂) and 7.40 ppm (1H, m, CONH₂); δ_{C} (CDCl₃) CH₃: 28.8, 28.8, 28.8, 45.6, 45.6; CH₂: 29.9, 53.9, 57.0; CH: 53.9; C: 80.2, 156.0, 174.9.

### Preparative Example 35

### 2(S)-AMINO-4-DIMETHYLAMINOBUTYRAMIDE

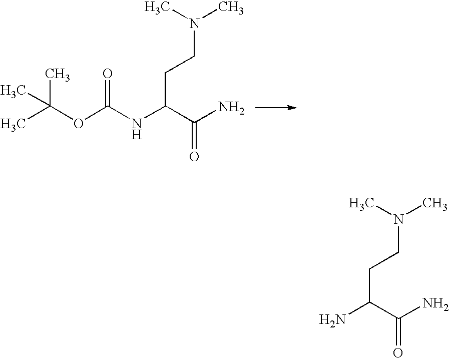

2(S)-tert-Butoxycarbonylamino-4-dimethylaminobutyramide (570 mg, 2.3 mmoles) (prepared as described in Preparative Example 34 above) was dissolved in anhydrous dichloromethane (40 mL) and the mixture was cooled to 0° C. under nitrogen with stirring. Tin (II) triflate (969.1 mg, 2.3 mmoles) was added in portions at 0° C. and the mixture stirred at 25° C. for 66 h, during which time a gummy solid separated. The reaction mixture was partitioned between dichloromethane and saturated aqueous sodium bicarbonate. The aqueous layer was extracted twice with dichloromethane (200 mL) and the latter was dried (MgSO₄), filtered and evaporated to dryness. The residue was taken up in methanol (2 mL) and 10% (v/v) concentrated sulfuric acid in dioxane (10 mL) was added and the mixture was stirred at 25° C. for 3 h. The reaction mixture was diluted with methanol and BioRad AG1X8 (OH⁻) resin was added until the pH reached 8. The resin was filtered off and washed with methanol. The combined filtrates were evaporated to dryness to give 2(S)-amino-4-dimethylaminobutyramide (38.7 mg, 11%): LCMS: m/z 146.1 (MH⁺).

### Preparative Example 36

### 2(S)-(+)-tert-BUTOXYCARBONYLAMINO-5-DIMETHYLAMINOPENTANOIC ACID

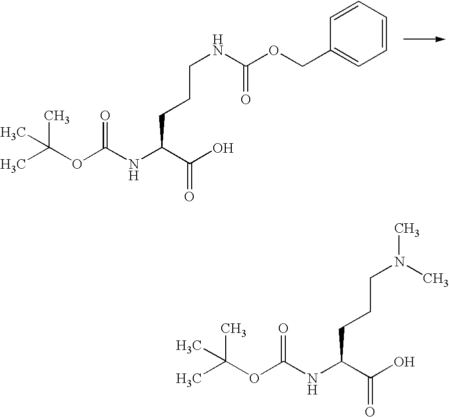

5-Benzyloxycarbonylamino-2(S)-tert-butoxycarbonylaminopentanoic acid (20 g, 54.6 mmoles) and 37% aqueous formaldehyde (130.1 mL, 131 mmoles) were dissolved in methanol-distilled water (1:1) (300 mL). 10% Pd—C (wet, ˜7 g) was added in portions under argon and the mixture was hydrogenated at 25° C. at 50 psi in a Parr hydrogenator for 4 days. The catalyst was filtered off through Celite© and the latter was washed with methanol-distilled water (1:1). The combined filtrates were evaporated to dryness to give 2(S)-(+)-tert-butoxycarbonylamino-4-dimethylaminopentanoic acid (14.21 g, 100%): ESMS: m/z 261.0 (MH⁺); Found: C, 55.15; H, 8.97: N, 10.38; C₁₂H₂₄N₂O₄ requires: C, 55.36; H, 9.29; N, 10.96; δ_{H} (CDCl₃) 1.40 (9H, s, COCC(CH₃)₃), 1.59 (1H, m, CHCH₂CH₂CH₂N(CH₃)₂) 1.77 (3H, m, CHCH₂CH₂CH₂N(CH₃)₂), 2.67 (6H, s, CHCH₂CH₂CH₂N(CH₃)₂), 2.77 (1H, m, CHCH₂CH₂CH₂N(CH₃)₂), 2.90 (1H, m, CHCH₂CH₂CH₂N(CH₃)₂), 4.06 (1H, m, CHCH₂CH₂CH₂N(CH₃)₂) and 5.68 ppm (1H, d, NH); δ_{C} (CDCl₃) CH₃: 28.5, 28.5, 28.5, 42.7, 42.7; CH₂: 21.0, 30.2, 57.9; CH: 54.5; C: 78.9, 155.5, 176.5; [α]_{D}^{25° C.} +23.2° (c=0.51, MeOH).

### Preparative Example 37

### 2(S)-(+)-tert-BUTOXYCARBONYLAMINO-5-DIMETHYLAMINOPENTANOIC ACID ISOBUTYL ESTER

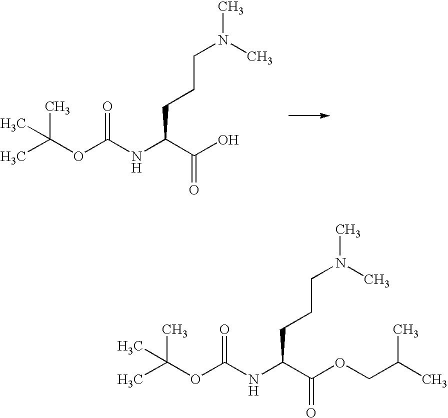

2(S)-(+)-tert-Butoxycarbonylamino-4-dimethylaminopentanoic acid (7 g, 26.9 mmoles) (prepared as described in Preparative Example 36 above), N-methylmorpholine (2.99 g, 3.25 mL, 29.6 mmoles) and isobutyl chloroformate (4.04 g, 3.84 mL, 26.9 mmoles) were dissolved in anhydrous THF (270 mL) and the mixture was stirred at −20° C. for 30 min. Conc. ammonium hydroxide (30%) (13.5 mL) was added and the mixture was stirred at 0° C. for 3 h. The mixture was evaporated to dryness and the product was chromatographed on a silica gel column (30×5 cm) using 5% (10% concentrated ammonium hydroxide in methanol)-dichloromethane as the eluant to give 2(S)-(+)-tert-butoxycarbonylamino-4-dimethylaminopentanoic acid isobutyl ester (6.48 g, 76%): FABMS: m/z 317.2 (MH⁺); HRFABMS: m/z 317.2437 (MH⁺). Calcd. for C₁₆H₃₃N₂O₄: m/z 317.2440; δ_{H} (CDCl₃) 0.93 (6H, d, COOCH₂CH(CH₃)₂), 1.42 (9H, s, COOC(CH₃)₃), 1.70-1.90 (2H, m, CHCH₂CH₂CH₂N(CH₃)₂), 1.94 (1H, d, COOCH₂CH(CH₃)₂), 2.58 (6H, s, CHCH₂CH₂CH₂N(CH₃)₂), 2.79 (2H, m, CHCH₂CH₂CH₂N(CH₃)₂), 3.92 (2H, d, COOCH₂CH(CH₃)₂), 4.26 (1H, m, CHCH₂CH₂CH₂N(CH₃)₂) and 5.52 ppm (1H, d, NH); δ_{C} (CDCl₃) CH₃: 19.1, 19.1, 28.4, 28.4, 28.4, 43.7, 43.7; CH₂: 21.4, 30.5, 57.8, 71.7; CH: 27.7, 52.7; C: 80.0, 155.8, 172.3; [α]_{D}^{25° C.} +19.9° (c=0.52, MeOH).

### Preparative Example 38

### 2(S)-AMINO-5-DIMETHYLAMINOPENTANOIC ACID ISOBUTYL ESTER

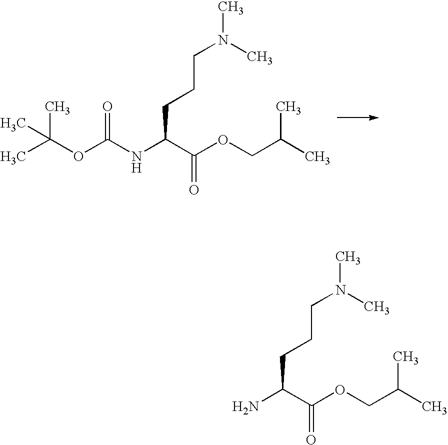

2(S)-(+)-tert-Butoxycarbonylamino-5-dimethylaminopentanoic acid isobutyl ester (1.0 g, 3.2 mmoles) (prepared as described in Preparative Example 37 above) was dissolved in anhydrous dichloromethane (100 mL) and the mixture was stirred at 0° C. under nitrogen. Tin (II) triflate (1.317 g, 3.2 mmoles) was added in portions at 0° C. and the mixture stirred at 25° C. for 23 h. The reaction mixture was partitioned between dichloromethane and saturated aqueous sodium bicarbonate. The dichloromethane extracts were dried (MgSO₄), filtered and evaporated to dryness. The residue was chromatographed on a silica gel column (30×2.5 cm) using 10% (10% concentrated ammonium hydroxide in methanol)-dichloromethane as the eluant to give 2(S)-(+)-amino-5-dimethylaminopentanoic acid methyl ester (142.8 mg, 21%): LCMS: m/z 217.1 (MH⁺); HRFABMS: m/z 217.1710 (MH⁺). Calcd. for C₁₁H₂₅N₂O₂: m/z 217.1916; δ_{H} (CDCl₃) 1.00 (6H, d, COOCH₂CH(CH₃)₂), 1.62 (2H, m, CHCH₂CH₂CH₂N(CH₃)₂), 1.77 (1H, m, CHCH₂CH₂CH₂N(CH₃)₂), 1.98 (1H, m, CHCH₂CH₂CH₂N(CH₃)₂), 2.28 (1H, m, COOCH₂CH(CH₃)₂), 2.31 (6H, s, N(CH₃)₂), 2.40 (2H, m, CHCH₂CH₂CH₂N(CH₃)₂), 3.50 (1H, m, CHCH₂CH₂CH₂N(CH₃)₂) and 3.96 ppm (2H, m, COOCH₂CH(CH₃)₂); δ_{C} (CDCl₃) CH₃: 18.4, 18.4, 44.2, 44.2; CH₂: 23.2, 32.5, 59.3, 71.1; CH: 28.0, 54.0; C, 175.4; [α]_{D}^{25° C.} +0.36° (c=0.88, MeOH).

### Preparative Example 39

### 2(S)-tert-BUTOXYCARBONYLAMINO-5-DIMETHYLAMINOPENTANOIC ACID METHYL ESTER

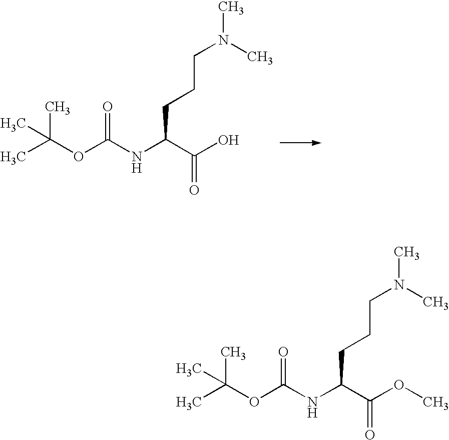

2(S)-(+)-tert-Butoxycarbonylamino-4-dimethylaminopentanoic acid (prepared as described in Preparative Example 38 above) may be reacted with either diazomethane, or trimethylsilyl diazomethane in a suitable inert solvent such as THF using methods well known to those skilled in the art, to give 2(S)-tert-butoxycarbonylamino -4-dimethylaminopentanoic acid methyl ester.

### Preparative Example 40

### 2(S)-AMINO-5-DIMETHYLAMINOPENTANOIC ACID METHYL ESTER

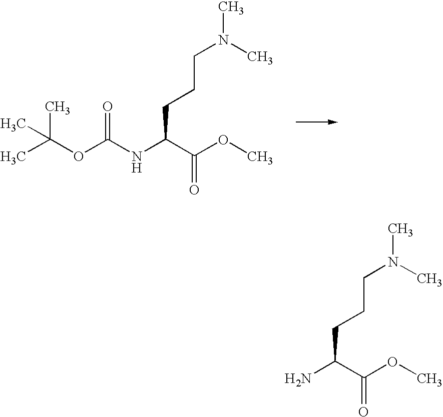

2(S)-(+)-tert-Butoxycarbonylamino-4-dimethylaminopentanoic acid methyl ester (prepared as described in Preparative Example 39 above) may be deprotected as described in Preparative Example 27, Step B to give 2(S)-amino-4-dimethylaminopentanoic acid methyl ester.

### Preparative Example 41

### [1-CARBAMOYL-4(S)-(+)-DIMETHYLAMINOBUTYL]CARBAMIC ACID tert-BUTYL ESTER

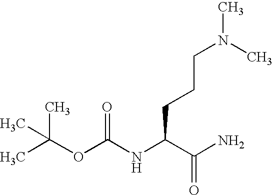

### A. [4(S)-(+)-tert-BUTOXYCARBONYLAMINO-4-CARBAMOYLBUTYL]CARBAMIC ACID BENZYL ESTER

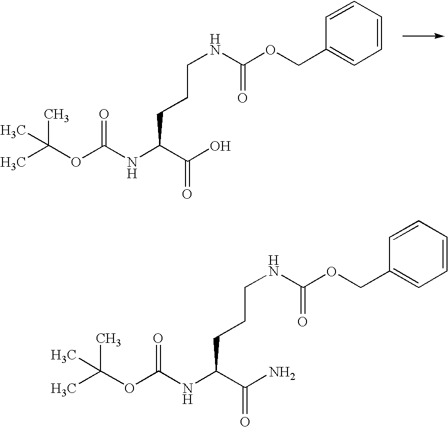

5-Benzyloxycarbonylamino-2(S)-tert-butoxycarbonylaminopentanoic acid (10 g, 27.3 mmoles), N-methylmorpholine (3.04 g, 3.3 mL, 30.0 mmoles) and isobutyl chloroformate (4.1 g, 3.89 mL, 30.0 mmoles) were dissolved in anhydrous THF (300 mL) and the mixture was stirred at −20° C. for 15 min. Conc. ammonium hydroxide (30%) (20 mL) was added and the mixture was stirred at −20 to 0° C. for 3 h. and then evaporated to dryness. The residue was chromatographed on a silica gel column (30×5 cm) using 5% (10% concentrated ammonium hydroxide in methanol)-dichloromethane as the eluant to give [4(S)-(+)-tert-butoxycarbonylamino -4-carbamoylbutyl]carbamic acid benzyl ester (9.93 g, 100%): ESMS: m/z 366.2 (MH⁺); HRFABMS: m/z 366.2032 (MH⁺). Calcd. for C₁₈H₂₈N₃O₅: m/z 366.2029; □_{H} (d₆-DMSO) 1.34 (9H, s, COOC(CH₃)₃), 1.38 (2H, m, NHCHCH₂CH₂CH₂NHCOO), 1.55 (1H, m, NHCHCH₂CH₂CH₂NHCOO), 2.48 (1H, m, NHCHCH₂CH₂CH₂NHCOO), 2.93 (2H, m, NHCHCH₂CH₂CH₂NHCOO), 3.78 (1H, m, NHCHCH₂CH₂CH₂NHCOO), 4.97 (2H, s, CH₂C₆H₅), 6.68 (1H, d, NHCHCH₂CH₂CH₂NHCOO), 6.92 (1H, d, NHCHCH₂CH₂CH₂NHCOO), 7.20 (2H, m, CH₂C₆H₅) and 7.32 ppm (3H, m, CH₂C₆H₅); □_{C} (d₆-DMSO)CH₃: 29.2, 29.2, 29.2; CH₂: 26.1, 29.4, 65.1; CH: 53.8, 127.7, 127.7, 128.4, 128.4, 128.4; C: 77.9, 137.3, 155.3, 156.1, 174.1; [α]_{D}^{25° C.} +4.1° (c=0.52, MeOH).

### B. [1-CARBAMOYL-4(S)-(+)-DIMETHYLAMINOBUTYL]CARBAMIC ACID tert-BUTYL ESTER and [4-DIMETHYLAMINO-1(S)-(−)-(HYDROXYMETHYLCARBAMOYL)BUTYLCARBAMIC ACID tert-BUTYL ESTER

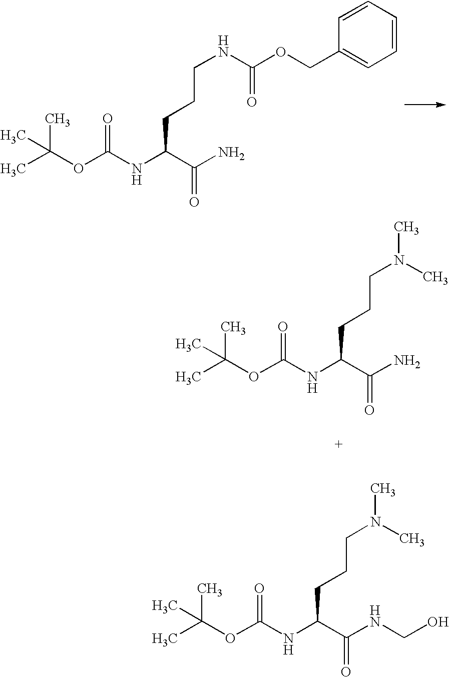

[4(S)-(+)-tert-Butoxycarbonylamino-4-carbamoylbutyl]carbamic acid benzyl ester (6 g, 16.4 mmoles) (prepared as described in Preparative Example 41, Step A above) was dissolved in methanol (150 mL) and distilled water (50 mL) and 37% aqueous formaldehyde (3.19 mL, 39.4 mmoles) was added. 10% Pd—C (wet, ˜3.5 g) was added in portions under argon and the mixture was hydrogenated at 25° C. and 50 psi in a Parr hydrogenator for 24 h. The catalyst was filtered off through Celite© and the latter was washed with methanol-distilled water (1:1). The combined filtrates were evaporated to dryness. The residue was chromatographed on a silica gel column (30×5 cm) using 7% (10% concentrated ammonium hydroxide in methanol)-dichloromethane as the eluant to give [1-carbamoyl-4(S)-(+)-dimethylaminobutyl]carbamic acid tert-butyl ester (3.33 g, 78%): FABMS: m/z 260.2 (MH⁺); HRFABMS: m/z 260.1982 (MH⁺). Calcd. for C₁₂H₂₆N₃O₃: m/z 260.1974; δ_{H} (CDCl₃) 1.43 (9H, s, COOC(CH₃)₃), 1.58 (2H, m, NHCHCH₂CH₂CH₂N(CH₃)₂), 1.80 (2H, m, NHCHCH₂CH₂CH₂N(CH₃)₂), 2.22 (6H, s, NHCHCH₂CH₂CH₂N(CH₃)₂), 2.31 (2H, m, NHCHCH₂CH₂CH₂N(CH₃)₂), 4.08 (H, m, NHCHCH₂CH₂CH₂N(CH₃)₂), 5.69 (1H, bs, NH), 6.60 (1H, bs, NH₂) and 6.72 ppm (1H, bs, NH₂); δ_{C} (CDCl₃) CH₃: 28.4, 28.4, 28.4; CH₂: 23.5, 30.8, 58.9; CH: 53.8; C: 79.7, 156.2, 174.8; [α]_{D}^{25° C.} +2.6° (c=0.50, MeOH) and [4-dimethylamino-1(S)-(−)-(hydroxymethylcarbamoyl)butylcarbamic tert-butyl ester (466.5 mg, 10%): FABMS: m/z 290.2 (MH⁺); HRFABMS; m/z 290.2092 (MH⁺). Calcd. for C₁₄H₂₈N₄O₃: m/z 290.2080; δ_{H} (CDCl₃) 1.43 (3H, s, COOC(CH₃)₃), 1.60 (2H, m, NHCHCH₂CH₂CH₂N(CH₃)₂), 1.77 (1H, m, NHCHCH₂CH₂CH₂N(CH₃)₂), 1.81 (1H, m, NHCHCH₂CH₂CH₂N(CH₃)₂), 2.24 (6H, s, NHCHCH₂CH₂CH₂N(CH₃)₂), 2.30 (1H, m, NHCHCH₂CH₂CH₂N(CH₃)₂), 2.42 (1H, m, NHCHCH₂CH₂CH₂N(CH₃)₂), 4.09 (1H, m, NHCHCH₂CH₂CH₂N(CH₃)₂), 4.78 (2H, m, CONHCH₂OH), 6.49 (1H, m, NHCHCH₂CH₂CH₂N(CH₃)₂) and 7.92 ppm (1H, bs, CONHCH₂OH); δ_{C} (CDCl₃) CH₃: 28.5, 28.5, 28.5; CH₂: 23.2, 30.8, 58.6, 64.5; CH: 53.8; C: 79.8, 156.2, ˜174.0; [α]_{D}^{25° C.} −6.2° (c=0.66, MeOH).

### Preparative Example 42

### 2(S)-(+)-AMINO-5-DIMETHYLAMINOPENTANAMIDE

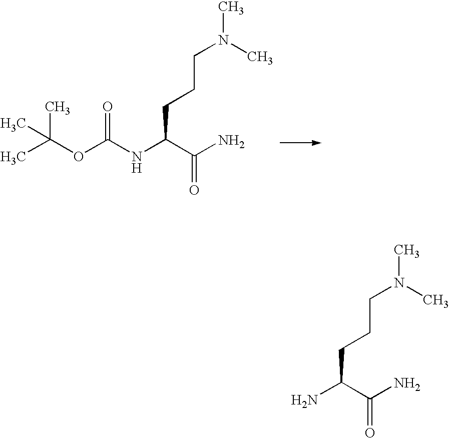

[1-Carbamoyl-4(S)-(+)-dimethylaminobutyl]carbamic acid tert-butyl ester (3.12 g, 12.0 mmoles) (prepared as described in Preparative Example 41, Step B above) was dissolved in methanol (15 mL) and 10% (v/v) concentrated sulfuric acid in dioxane (50 mL) was added. The mixture was stirred at 25° C. for 3 h and then diluted with methanol. BioRad AG1X8 (OH⁻) resin was added until the pH reached 8. The resin was filtered off and washed with methanol and the combined filtrates were evaporated to dryness. The residue was chromatographed on a silica gel column (15×5 cm) using 10% (10% concentrated ammonium hydroxide in methanol)-dichloromethane as the eluant to give 2(S)-(+)-amino-5-dimethylaminopentanamide (592 mg, 31%): LCMS: m/z 160.1 (MH⁺); HRFABMS: m/z 160.1457 (MH⁺). Calcd. for C₇H₁₈N₃O: m/z 160.1450; δ_{H} (CDCl₃) 1.70 (2H, m, CHCH₂CH₂CH₂N(CH₃)₂), 1.70 (1H, m, CHCH₂CH₂CH₂N(CH₃)₂), 1.83 (1H, m, CHCH₂CH₂CH₂N(CH₃)₂), 2.47 (6H, s, N(CH₃)₂), 2.62 (2H, m, CHCH₂CH₂CH₂N(CH₃)₂) and 3.72 ppm (1H, m, CHCH₂CH₂CH₂N(CH₃)₂); δ_{C} (CDCl₃) CH₃: 43.5, 43.5; CH₂: 22.8, 33.0, 58.7; CH: 54.2; C, 176.8; [α]_{D}^{25° C.} +4.07° (c=1.10, MeOH).

### Preparative Example 43

### 2(S)-(+)-tert-BUTOXYCARBONYLAMINO-6-DIMETHYLAMINOHEXANOIC ACID

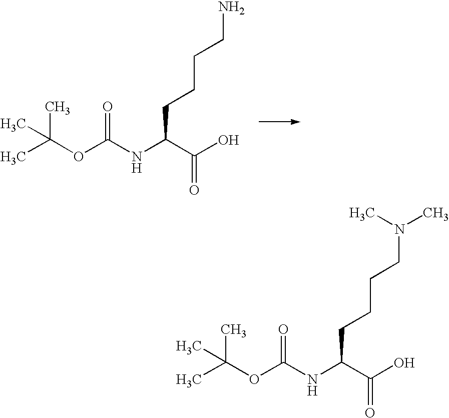

2-tert-Butoxycarbonyl-(S)-(+)-lysine (20 g, 81.2 mmoles) and 37% aqueous formaldehyde (19.5 mL, 19.5 mmoles) were dissolved in distilled water (300 mL). 10% Pd—C (wet, ˜7 g) was added in portions under argon and the mixture was hydrogenated at 25° C. and 50 psi in a Parr hydrogenator for 4 days The catalyst was filtered off through Celite© and the latter was washed with methanol-distilled water (1:1). The combined filtrates were evaporated to dryness to give 2(S)-(+)-tert-butoxycarbonylamino-6-dimethylaminohexanoic acid (22.53 g, 100%): ESMS: m/z 275.0 (MH⁺); Found: C, 55.08; H, 9.64; N, 9.69; C₁₃H₂₆N₂O₄ requires: C, 56.91; H, 9.55; N, 10.21; δ_{H} (CDCl₃) 1.32 (1H, m, NHCHCH₂CH₂CH₂CH₂N(CH₃)₂), 1.42 (9H, s, COOC(CH₃)₃), 1.44 (1H, m, NHCHCH₂CH₂CH₂CH₂N(CH₃)₂), 1.70 (2H, m, NHCHCH₂CH₂CH₂CH₂N(CH₃)₂), 1.79 (1H, m, NHCHCH₂CH₂CH₂CH₂N(CH₃)₂), 1.90 (1H, m, NHCHCH₂CH₂CH₂CH₂N(CH₃)₂), 2.68 (6H, s, NHCHCH₂CH₂CH₂CH₂N(CH₃)₂), 2.80 (1H, m, NHCHCH₂CH₂CH₂CH₂N(CH₃)₂), 2.88 (1H, m, NHCHCH₂CH₂CH₂CH₂N(CH₃)₂), 4.08 (1H, m, NHCHCH₂CH₂CH₂CH₂N(CH₃)₂) and 5.62 ppm (1H, d, NHCHCH₂CH₂CH₂CH₂N(CH₃)₂); δ_{C} (CDCl₃) CH₃: 28.3, 28.3, 28.3; CH₂: 22.2, 25.0, 32.8, 57.5; CH: 54.6; C: 78.7, 155.5, 177.2; [α]_{D}^{25° C.} +18.5° (c=0.52, MeOH).

### Preparative Example 44

### 2-tert-BUTOXYCARBONYLAMINO-6-DIMETHYLAMINOHEXANOIC ACID METHYL ESTER

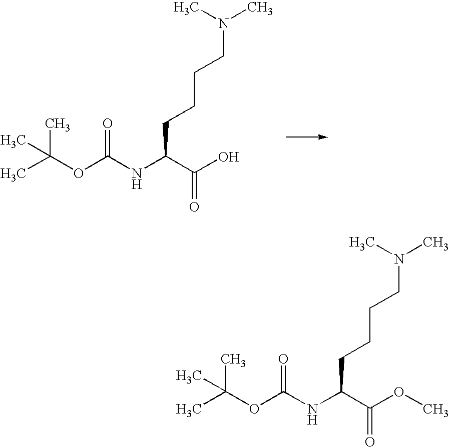

2(S)-(+)-tert-Butoxycarbonylamino-6-dimethylaminohexanoic acid (prepared as described in Preparative Example 43 above) may be reacted with either diazomethane, or trimethylsilyl diazomethane in a suitable inert solvent such as THF using methods well known to those skilled in the art, to give 2(S)-tert-butoxycarbonylamino-6-dimethylaminohexanoic acid methyl ester.

### Preparative Example 45

### 2-AMINO-6-DIMETHYLAMINOHEXANOIC ACID METHYL ESTER

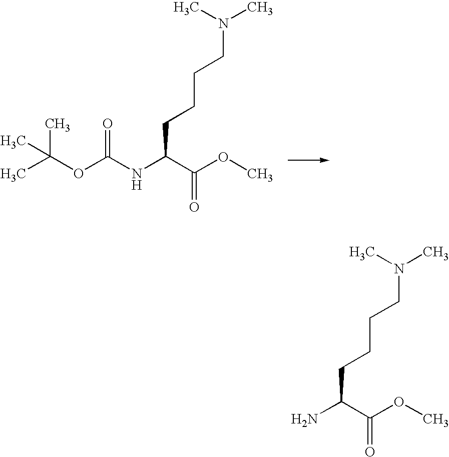

2(S)-tert-Butoxycarbonylamino-6-dimethylaminohexanoic acid methyl ester (prepared as described in Preparative Example 45 above) may be deprotected as described in Preparative Example 27, Step B to give 2(S)-amino-6-dimethylaminohexanoic acid methyl ester.

### Preparative Example 46

### [1(S)-(+)-CARBAMOYL-5-DIMETHYLAMINOPENTYL]CARBAMIC ACID tert-BUTYL ESTER

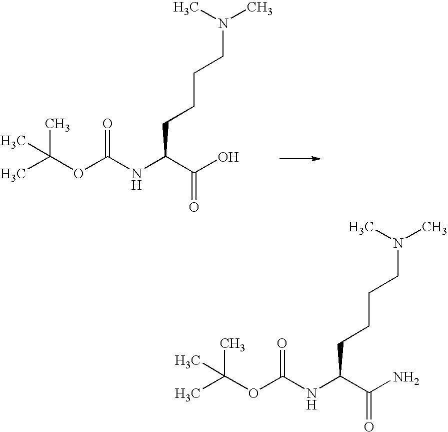

2(S)-(+)-tert-Butoxycarbonylamino-6-dimethylaminohexanoic acid (10 g, 36.4 mmoles) (prepared as described in Preparative Example 43 above), N-methylmorpholine (4.06 g, 4.41 mL, 40.1 mmoles) and isobutyl chloroformate (5.48 g, 5.2 mL, 40.1 mmoles) were dissolved in anhydrous THF (370 mL) and the mixture was stirred at −20° C. for 30 min. Conc. ammonium hydroxide (30%) (18.5 mL) was added and the mixture was stirred at 0° C. for 3 h. The mixture was evaporated to dryness and the product was chromatographed on a silica gel column (30×5 cm) using 7% (10% concentrated ammonium hydroxide in methanol)-dichloromethane as the eluant to give [1(S)-(+)-carbamoyl-5-dimethylaminopentyl]carbamic acid tert-butyl ester (8.81 g, 88%): FABMS: m/z 274.2 (MH⁺); HRFABMS: m/z 274.2129 (MH⁺). Calcd. for C₁₃H₂₈N₃O₃: m/z 274.2131; δ_{H} (CDCl₃) 1.43 (2H, m, NHCHCH₂CH₂CH₂CH₂N(CH₃)₂), 1.43 (9H, s, COOC(CH₃)₃), 1.58 (2H, m, NHCHCH₂CH₂CH₂CH₂N(CH₃)₂), 1.67 (1H, m, NHCHCH₂CH₂CH₂CH₂N(CH₃)₂), 1.84 (1H, m, NHCHCH₂CH₂CH₂CH₂N(CH₃)₂), 2.32 (6H, s, NHCHCH₂CH₂CH₂CH₂N(CH₃)₂), 2.42 (2H, m, NHCHCH₂CH₂CH₂CH₂N(CH₃)₂), 4.13 (1H, m, NHCHCH₂CH₂CH₂CH₂N(CH₃)₂), 5.45 ppm (1H, d, NHCHCH₂CH₂CH₂CH₂N(CH₃)₂), 5.84 (1H, bs, CONH₂) and 6.69 ppm (1H, bs, CONH₂); δ_{C} (CDCl₃) CH₃: 28.4, 28.4, 28.4; CH₂: 23.0, 26.4, 32.1, 58.9; CH: 53.9; C; 80.0, 155.9, 174.8; [α]_{D}^{25° C.} +2.2° (c=0.52, MeOH).

### Preparative Example 47

### 2(S)-(+)-AMINO-6-DIMETHYLAMINOHEXANOIC ACID AMIDE

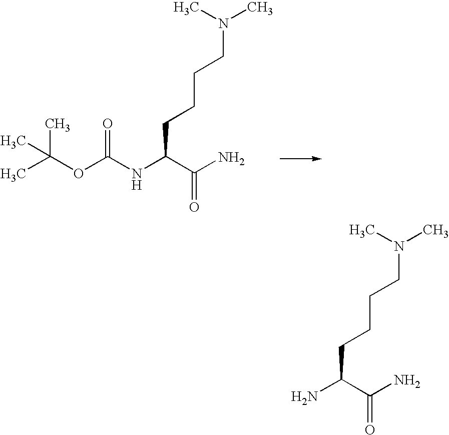

[1(S)-(+)-Carbamoyl-6-dimethylaminopentyl]carbamic acid tert-butyl ester (prepared as described in Preparative Example 46 above) may be deprotected as described in Preparative Example 27, Step B to give 2(S)-amino-5-dimethylaminohexanoic acid amide.

### Example 1

### N′-(2-{4-[BIS-(4-CHLOROPHENYL)METHYL]PIPERAZIN-1-YLMETHYL}QUINAZOLIN-4-YL)-N,N-DIMETHYLPROPANE-1,3-DIAMINE

Method 1:

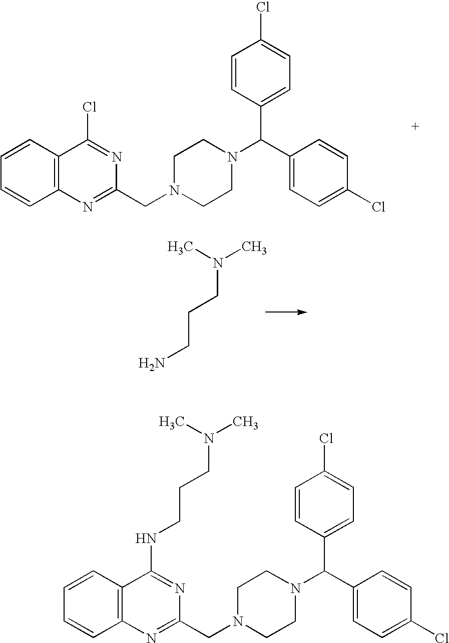

2-{4-[Bis-(4-chlorophenyl)methylpiperazin-1-ylmethyl}4-chloroquinazoline (160 mg, 0.32 mmoles) (prepared as described in Preparative Example 8 above) and 3-dimethylaminopropylamine (65.7 mg, 0.0811 mL, 0.64 mmoles) were dissolved in 200 proof ethanol (10 mL) and the mixture was heated under argon at 80° C. for 21 h. The solution was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column 30×2.5 cm) using 5% (10% concentrated ammonium hydroxide in methanol)-dichloromethane as the eluant to give N′-(2-{4-[bis-(4-chlorophenyl)methyl]piperazin-1-ylmethyl}quinazolin-4-yl)-N,N-dimethylpropane-1,3-diamine (126 mg, 70%): FABMS: m/z 563.4 (MH⁺); HRFABMS: m/z 563.2449 (MH⁺). Calcd. for C₃₁H₃₇Cl₂N₆: m/z 563.2457; Found: C, 64.95; H, 6.51; Cl, 11.52; N, 14.39. C₃₁H₃₆Cl₂N₆ requires: C, 66.07; H, 6.44; Cl, 12.58; N, 14.91; δ_{H} (CDCl₃) 1.84 (2H, m, NHCH₂CH₂CH₂N(CH₃)₂), 2.38 (6H, s, NHCH₂CH₂CH₂N(CH₃)₂), 2.46 (4H, m, N(CH₂CH₂)N), 2.60 (4H, m, NHCH₂CH₂CH₂N(CH₃)₂), 2.76 (4H, m, N(CH₂CH₂)N), 3.74 (4H, s, NHCH₂CH₂CH₂N(CH₃)₂), 4.20 (1H, s, NCH(C₆H₄Cl)₂), 7.23 (4H, d, NCH(C₆H₄Cl)₂), 7.32 (4H, d, NCH(C₆H₄Cl)₂), 7.38 (1H, ddd, H₆), 7.59 (1H, dd, H₅), 7.65 (1H, ddd, H₇), 7.78 (1H, dd, H₈) and 8.63 ppm (1H, bm, NH); δ_{C} (CDCl₃) CH₃: 45.6, 45.6; CH₂: 24.7, 42.6, 51.9, 51.9, 53.7, 53.7, 60.0, 65.4; CH: 74.9, 120.9, 125.2, 128.3, 128.8, 128.8, 128.8, 128.8, 129.2, 129.2, 129.2, 129.2, 132.0; C: 114.1, 132.7, 132.7, 141.1, 141.1, 149.9, 159.8, 163.5.
Method 2:

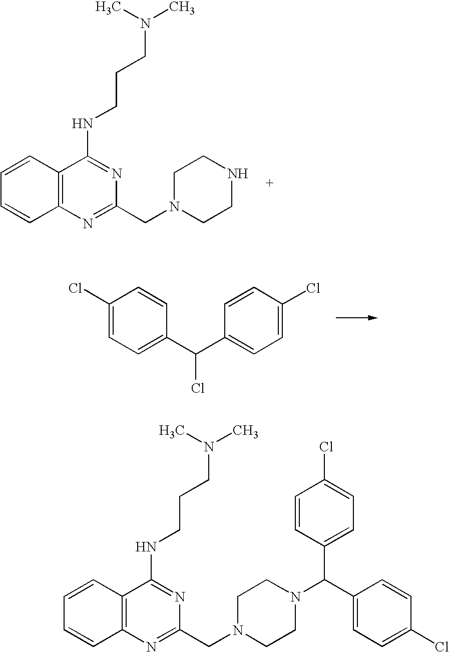

N,N-Dimethyl-N′-(2-piperazin-1-ylmethylquinazolin-4-yl)propane-1,3-diamine (9 g, 27.4 mmoles) (prepared as described in Preparative Example 24 above), anhydrous potassium carbonate (3.79 g, 27.4 mmoles) and anhydrous potassium iodide (4.55 g, 27.4 mmoles) were dissolved in anhydrous acetonitrile (41 mL) and a solution of bis-(4-chlorophenyl)methyl chloride (14.9 g, 54.8 mmoles) [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)] in anhydrous acetonitrile (87 mL) was added. The mixture was stirred at 25° C. for 117 h. The mixture was evaporated to dryness and the residue was partitioned between dichloromethane and water. The organic layer was dried (MgSO₄), filtered and evaporated to dryness. The residue was chromatographed on a silica gel column (45×8 cm) using 5% (10% concentrated ammonium hydroxide in methanol)-dichloromethane as the eluant to give N′-(2-{4-[bis-(4-chlorophenyl)methyl]piperazin-1-ylmethyl}quinazolin-4-yl)-N,N-dimethylpropane-1,3-diamine (7.87 g, 51%).

N′-(2-{4-[Bis-(4-chlorophenyl)methyl]piperazin-1-ylmethyl}quinazolin-4-yl)-N,N-dimethylpropane-1,3-diamine (1.72 g, 3.06 mmoles) was dissolved in anhydrous dichloromethane (52 mL) and 4.0M HCl in 1,4-dioxane (3.83 mL, 15.3 mmoles) was added dropwise and the mixture was stirred at 25° C. for 20 min. The solution was evaporated to dryness and the hydrochloride salt was dried in vacuo at 25° C. for 67 h (2.33 g): Found: C, 50.97; H, 6.19; Cl, 25.57; N, 10.36 (C₃₁H₃₆Cl₂N₆.4.5HCl.1.1C₄H₈O₂).

The compound was found to have % Residual T @ 2 ug/mL rating according to scintillation proximity assay (SPA) of “A” and an EC50 value according to the proliferation assay (MB468) rating of “A”. (See descriptions of assays below).

### Example 2

### 2(S)-(−)-(2-{4-[BIS-(4-CHLOROPHENYL)METHYL]PIPERAZIN-1-YLMETHYL}QUINAZOLIN-4-YLAMINO)-3-METHYLBUTYRIC ACID METHYL ESTER

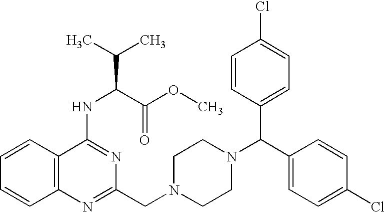 Method 1:

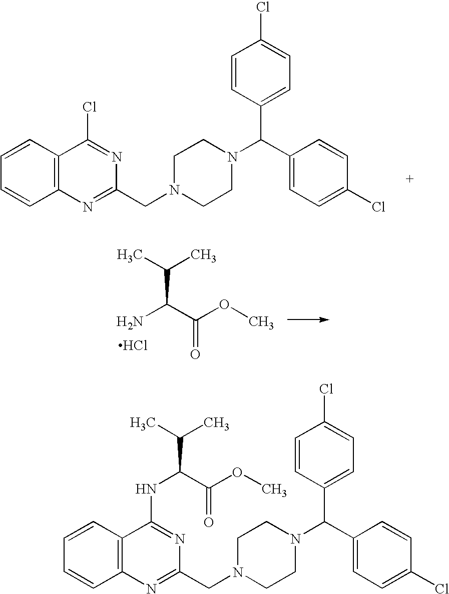

2-{4-[Bis-(4-chlorophenyl)methylpiperazin-1-ylmethyl}-4-chloroquinazoline (160 mg, 0.32 mmoles) (prepared as described in Preparative Example 8 above), 2(S)-(+)-valine methyl ester hydrochloride (107.8 mg, 0.64 mmoles) and triethylamine (195.2 mg, 0.268 mL, 1.92 mmoles) were added to anhydrous 1,4-dioxane (10 mL) and the slurry was heated under argon at 102° C. for 24 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (30×2.5 cm) using 0.5% (10% concentrated ammonium hydroxide in methanol)-dichloromethane as the eluant to give 2(S)-(−)-(2-{4-[bis-(4-chlorophenyl)methyl]piperazin-1-ylmethyl}quinazolin-4-ylamino)-3-methylbutyric acid methyl ester (103.5 mg, 54%): FABMS: m/z 592.3 (MH⁺).
Method 2:

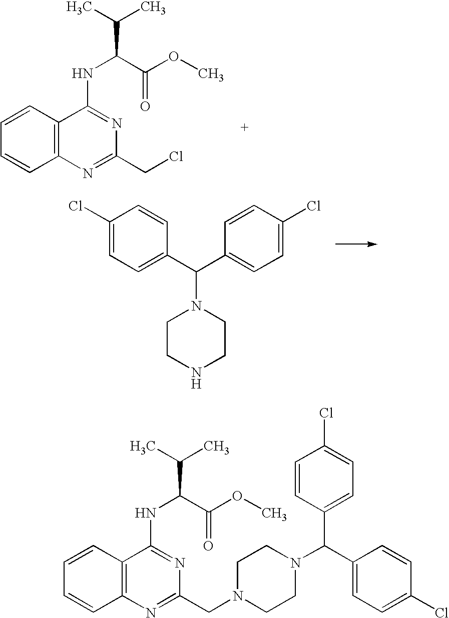

2(S)-(+)-(2-Chloromethylquinazolin-4-ylamino)-3-methylbutyric acid methyl ester (871.9 mg, 2.83 mmoles) (prepared as described in Preparative Example 9 above), 1-(4,4′-dichlorobenzhydryl)piperazine (910 mg, 2.83 mmoles) (prepared as described in Preparative Example 1 above) and anhydrous potassium carbonate (391.51 mg, 2.83 mmoles) were added to anhydrous acetonitrile (50 mL) and the mixture was heated under argon at 80° C. for 18 h. The mixture was evaporated to dryness and the residue was partitioned between dichloromethane and distilled water. The organic layer was dried (MgSO₄), filtered and evaporated to dryness. The residue was chromatographed on a silica gel column (30×5 cm) using 2% (conc. ammonium hydroxide in methanol)-dichloromethane as the eluant to give 2(S)-(−)-(2-{4-[bis-(4-chlorophenyl)methyl]piperazin-1-ylmethyl}quinazolin-4-ylamino)-3-methylbutyric acid methyl ester (1.53 g, 91%): FABMS: m/z 592.4 (MH⁺); HRFABMS: m/z 592.2258 (MH⁺). Calcd. for C₃₂H₃₆Cl₂N₅O₂: m/z 592.2246; Found: C, 63.99; H, 5.64; Cl, 11.65; N, 11.71. C₃₂H₃₅Cl₂N₅O₂ requires: C, 64.86; H, 5.95; Cl, 11.97; N, 11.82; δ_{H} (CDCl₃) 1.08 (3H, d, NHCHCH(CH₃)₂), 1.12 (3H, d, NHCHCH(CH₃)₂), 2.42 (1H, m, NHCHCH(CH₃)₂), 2.50 (4H, m, N(CH₂CH₂)N), 2.80 (4H, m, N(CH₂CH₂)N), 3.82 (3H, s, COOCH₃), 4.27 (2H, s, 2-CH₂N), 5.15 (1H, m, NHCHCH(CH₃)₂), 6.24 (1H, d, NHCHCH(CH₃)₂), 7.28 (4H, ddd, NCH(C₆H₄Cl)₂), 7.36 (4H, ddd, NCH(C₆H₄Cl)₂), 7.50 (4H, ddd, NCH(C₆H₄Cl)₂), 7.50 (1H, ddd, H₆), 7.76 (1H, ddd, H₇), 7.83 (1H, dd, H₅) and 7.89 ppm (1H, dd, H₈); δ_{C} (CDCl₃) CH₃: 18.5, 19.0, 58.4; CH₂: 51.7, 51.7, 53.4, 53.4, 65.1; CH: 31.5, 52.2, 74.7, 120.5, 125.8, 128.7, 128.7, 128.7, 128.7, 128.7, 129.2, 129.2, 129.2, 129.2, 132.7; C: 113.8, 132.8, 132.8, 141.0, 141.0, 150.1, 159.1, ˜163.1, 173.1; [α]_{D}²⁵° C. −19.6° (c=0.42, MeOH).

### Example 3

### 2-(2-{4-[BIS-(4-CHLOROPHENYL)METHYL]PIPERAZIN-1-YLMIETHYL}QUINAZOLIN-4-YLAMINO)-3-MIETHYLBUTYRAMIDE

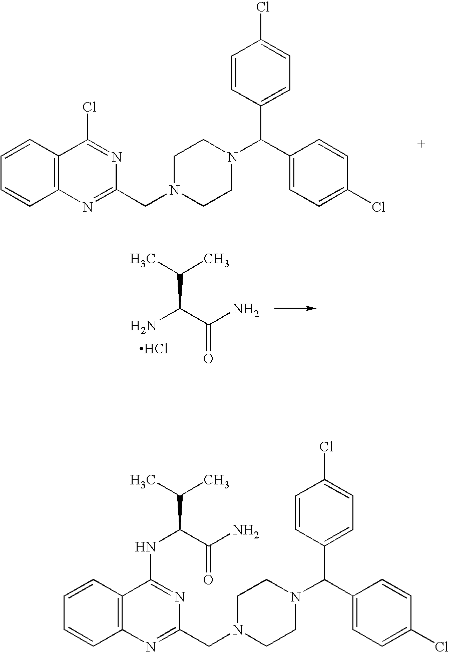

2-{4-[Bis-(4-chlorophenyl)methylpiperazin-1-ylmethyl}-4-chloroquinazoline (160 mg, 0.32 mmoles) (prepared as described in Preparative Example 8 above), 2(S)-(+)-valinamide hydrochloride (98 mg, 0.64 mmoles) and triethylamine (195.2 mg, 0.268 mL, 1.92 mmoles) were added to anhydrous 1,4-dioxane (10 mL) and the slurry was heated under argon at 102° C. for 24 h. The mixture was evaporated to dryness and the residue was taken up in dichloromethane and washed with saturated aqueous sodium bicarbonate. The organic layer was dried (MgSO₄), filtered and evaporated to dryness. The residue was chromatographed on a silica gel column (30×2.5 cm) using 2% (10% concentrated ammonium hydroxide in methanol)-dichloromethane as the eluant to give 2(S)-(−)-(2-{4-[bis-(4-chlorophenyl)methyl]piperazin-1-ylmethyl}quinazolin-4-ylamino)-3-methylbutyramide (77.7 mg, 42%): FABMS: m/z 577.3 (MH⁺); Found: C, 63.25; H, 5.70; Cl, 12.77; N, 14.15. C₃₁H₃₄Cl₂N₆O requires: C, 64.47; H, 5.93; Cl, 12.28; N, 14.55; δ_{H} (CDCl₃) 1.07 (6H, d, NHCHCH(CH₃)₂), 2.40 (4H, m, N(CH₂CH₂)N), 2.65 (4H, m, N(CH₂CH₂)N), 3.66/3.78 (2H, AB system, 2-CH₂N), 4.18 (1H, s, NCH(C₆H₄Cl)₂), 4.72 (1H, d, NHCHCH(CH₃)₂), 5.70 (1H, bs, CONH₂), 6.43 (1H, d, NHCHCH(CH₃)₂), 6.99 (1H, bs, CONH₂), 7.27 (4H, d, NCH(C₆H₄Cl)₂), 7.30 (4H, d, NCH(C₆H₄Cl)₂), 7.43 (1H, ddd, H₆), 7.72 (1H, ddd, H₇), 7.78 (1H, dd, H₅) and 7.80 ppm (1H, dd, H₈); δ_{C} (CDCl₃) CH₃: 18.8, 19.6; CH₂: 51.9, 51.9, 53.6, 53.6, 65.4; CH: 30.0, 59.6, 74.9, 120.8, 125.9, 128.5, 128.9, 128.9, 128.9, 128.9, 129.1, 129.1, 129.1, 129.1, 132.9; C: 113.6, 132.9, 132.9, 140.9, 140.9, 150.0, 159.5, 162.7, 174.1; [α]_{D}^{25° C.} −12.5° (c=0.50, MeOH).

### Example 4

### [2(S)-(−)-[4-[BIS-(4-CHLOROPHENYL)METHYL]-4-QUINAZOLINYL]AMINO]-N-METHOXY-3-METHYLBUTANAMIDE and (−)-[2-[[4-[BIS-(4-CHLOROPHENYL)METHYL]-4-QUINAZOLINYL]AMINO]-[2-[N-METHOXY-3-METHYLBUTANAMIDO]]-3-METHYL-BUTANAMIDE (ISOMER 1) and (+)-[2-[[4-[BIS-(4-CHLOROPHENYL)METHYL]-4-QUINAZOLINYL]AMINO]-[2-[N-METHOXY-3-METHYLBUTANAMIDO]]-3-METHYL-BUTANAMIDE (ISOMER 2)

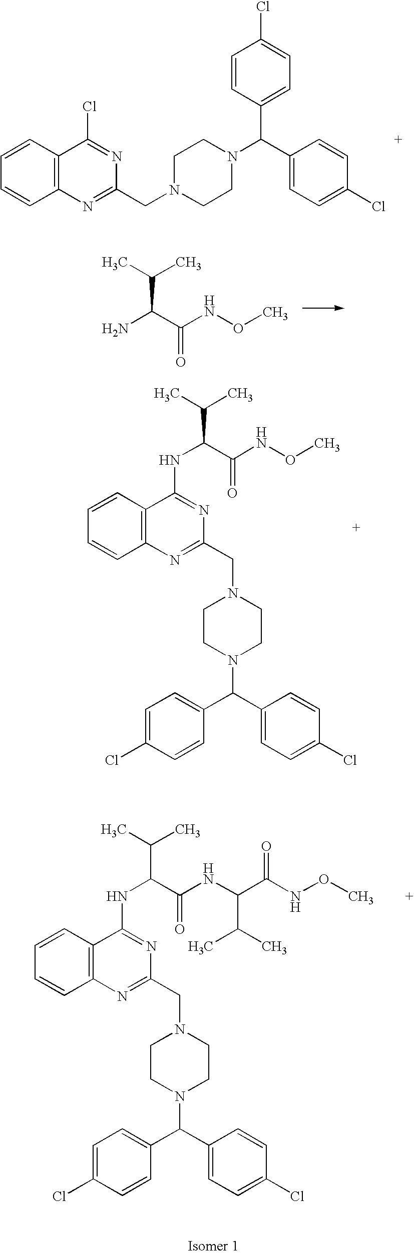 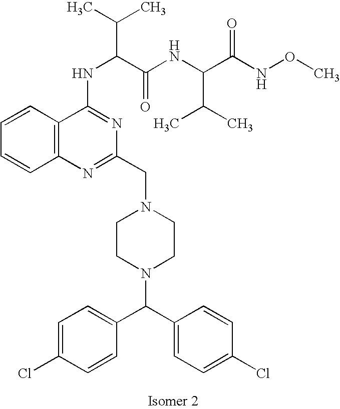 Method 1:

2-{4-[Bis-(4-chlorophenyl)methylpiperazin-1-ylmethyl}-4-chloroquinazoline (160 mg, 0.32 mmoles) (prepared as described in Preparative Example 8 above), (S)-(+)-amino-N-methoxy-3-methylbutyramide (94 mg, 0.64 mmoles) and triethylamine (195.2 mg, 0.268 mL, 1.92 mmoles) were added to anhydrous 1,4-dioxane (10 mL) and the slurry was heated under argon and under reflux at 102° C. for 24 h. The mixture was evaporated to dryness and the residue was taken up in dichloromethane and washed with saturated aqueous sodium bicarbonate. The organic layer was dried (MgSO₄), filtered and evaporated to dryness. The residue was first chromatographed on a silica gel column (30×2.5 cm) using 1% (10% concentrated ammonium hydroxide in methanol)-dichloromethane as the eluant and the three products were then further purified by preparative tic on 20×20 cm 250μ silica gel plates using 5% (10% concentrated ammonium hydroxide in methanol)-dichloromethane as the eluant to give in the order of elution, [2(S)-(−)-[[4-[bis(4-chlorophenyl)methyl]-4-quinazolinyl]amino]-N-methoxy-3-methylbutanamide (16.7 mg, 9%): FABMS: m/z 607.6 (MH⁺); HRFABMS: m/z 607.2343 (MH⁺). Calcd. for C₃₂H₃₇Cl₂N₆O₂: m/z 607.2355; δ_{H} (CDCl₃) 1.08 (6H, d, NHCHCH(CH₃)₂), 2.47 (4H, m, N(CH₂CH₂)N), 2.75 (4H, m, N(CH₂CH₂)N), 3.68 (3H, s, NHOCH₃), 3.65/3.76 (2H, AB system, 2-CH₂N), 4.24 (1H, s, NCH(C₆H₄Cl)₂), 4.24 (1H, d, NHCHCH(CH₃)₂), 6.39 (1H, d, NHCHCH(CH₃)₂), 7.25 (4H, d, NCH(C₆H₄Cl)₂), 7.31 (4H, d, NCH(C₆H₄Cl)₂), 7.38 (1H, ddd, H₆), 7.68 (1H, ddd, H₇), 7.73 (1H, dd, H₅) and 7.73 ppm (1H, dd, H₈); δ_{C} (CDCl₃) CH₃: 19.4/19.7, 19.4/19.7, 54.0; CH₂: 51.4/51.9, 51.4/51.9, 53.7/54.0, 53.7/54.0, 65.2; CH: 29.0, 58.8/63.9, 74.7, 120.9, 126.0, 128.3, 128.9, 128.9, 128.9, 128.9, 129.2, 129.2, 129.2, 129.2, 133.0; C: 113.4, 133.1, 133.1, 140.7, 140.7, 150.0, 159.6, 162.2; [α]_{D}^{25° C.} −23.2° (c=0.44, MeOH), then (−)-[2-[[4-[bis-(4-chlorophenyl)methyl]-4-quinazolinyl]amino]-[2-[N-methoxy-3-methylbutanamido]]-3-methylbutanamide (isomer 1) (20.2 mg, 9%): FABMS: m/z 706.4 (MH⁺); HRFABMS: m/z 706.3028 (MH⁺). Calcd. for C₃₇H₄₆Cl₂N₇O₃: m/z 706.3039; δ_{H} (CDCl₃) 0.80 (3H, d, NHCHCH(CH₃)₂), 0.89 (3H, d, NHCHCH(CH₃)₂), 1.01 (3H, d, NHCHCH(CH₃)₂), 1.05 (3H, d, NHCHCH(CH₃)₂), 2.48 (4H, m, N(CH₂CH₂)N), 2.69 (4H, m, N(CH₂CH₂)N), 3.73 (3H, s, NHOCH₃), 3.71/3.79 (2H, AB system, 2-CH₂N), 4.03 (1H, d, NHCHCH(CH₃)₂), 4.22 (1H, s, NCH(C₆H₄Cl)₂), 4.73 (1H, d, NHCHCH(CH₃)₂), 6.23 (1H, d, NHCHCH(CH₃)₂), 7.24 (4H, d, NCH(C₆H₄Cl)₂), 7.32 (4H, d, NCH(C₆H₄Cl)₂), 7.42 (1H, ddd, H₆), 7.71 (1H, ddd, H₇), 7.76 (1H, dd, H₅) and 8.03 ppm (1H, dd, H₈); δ_{C} (CDCl₃) CH₃: 18.5/18.8, 19.3/19.5, 56.7; CH₂: 51.6, 51.6, 53.7, 53.7, 65.2; CH: 30.1/30.7, 60.1/64.4, 74.8, 120.6, 126.9, 128.7, 128.8, 128.8, 128.8, 128.8, 129.2, 129.2, 129.2, 129.2, 133.0; C: 113.4, 132.9, 132.9, 140.9, 140.9, 150.1, 159.4, 162.3, 172.2; [α]_{D}^{25° C.} −37.6° (c=0.55, MeOH) and finally (+)-[2-[[4-[bis-(4-chlorophenyl)methyl]-4-quinazolinyl]amino]-[2-[N-methoxy-3-methylbutanamido]]-3-methyl-butanamide (isomer 2) (44.2 mg, 19%): FABMS: m/z 706.4 (MH⁺); HRFABMS: m/z 706.3026 (MH⁺). Calcd. for C₃₇H₄₆Cl₂N₇O₃: m/z 706.3039; δ_{H} (CDCl₃) 0.96 (3H, d, NHCHCH(CH₃)₂), 0.92 (3H, d, NHCHCH(CH₃)₂), 1.01 (6H, d, NHCHCH(CH₃)₂), 2.45 (4H, m, N(CH₂CH₂)N), 2.68 (4H, m, N(CH₂CH₂)N), 3.52 (3H, s, NHOCH₃), 3.66/3.88 (2H, AB system, 2-CH₂N), 4.12 (1H, d, NHCHCH(CH₃)₂), 4.22 (1H, s, NCH(C₆H₄Cl)₂), 4.69 (1H, d, NHCHCH(CH₃)₂), 6.38 (1H, d, NHCHCH(CH₃)₂), 7.23 (4H, d, NCH(C₆H₄Cl)₂), 7.31 (4H, d, NCH(C₆H₄C[)₂), 7.38 (1H, ddd, H₆), 7.68 (1H, ddd, H₇), 7.72 (1H, dd, H₅) and 7.79 ppm (1H, dd, H₈); δ_{C} (CDCl₃) CH₃: 18.6/18.8, 19.5, 56.9; CH₂: 51.5, 51.5, 53.6, 53.6, 65.3; CH: 30.2/30.9, 60.3/64.0, 74.7, 126.0, 127.0, 128.5, 128.9, 128.9, 128.9, 128.9, 129.2, 129.2, 129.2, 129.2, 132.9; C: 113.4, 133.8, 133.8, 140.8, 140.8, 149.9, 159.5, 162.4, 172.6; [α]_{D}^{25° C.} +18.7° (c=0.53, MeOH).

Method 2:

2-{4-[Bis-(4-chlorophenyl)methylpiperazin-1-ylmethyl}-4-chloroquinazoline (160 mg, 0.32 mmoles) (prepared as described in Preparative Example 8 above), (S)-(+)-amino-N-methoxy-3-methylbutyramide (47 mg, 0.32 mmoles) and triethylamine (97.6 mg, 0.134 mL, 0.96 mmoles) were added to anhydrous 1,4-dioxane (10 mL) and the slurry was heated underargon at 102° C. for 24 h. The mixture was evaporated to dryness and the residue was taken up in dichloromethane and washed with saturated aqueous sodium bicarbonate. The organic layer was dried (MgSO₄), filtered and evaporated to dryness. The residue was by preparative tic on 20×20 cm 250μ silica gel plates using 5% (10% concentrated ammonium hydroxide in methanol)-dichloromethane as the eluant to give [2(S)-(−)-[[4-[bis(4-chlorophenyl)methyl]-4-quinazolinyl]amino]-N-methoxy-3-methylbutanamide (42.1 mg, 22%).

### Example 5

### [2(S)-(−)-[[4-[BIS-(4-CHLOROPHENYL)METHYL]-4-QUINAZOLINYL]AMINO]-N-ETHOXY-3-METHYL-(2S)-BUTANAMIDE and (−)-[2-[[4-[BIS-(4-CHLOROPHENYL)METHYL]-4-QUINAZOLINYL]AMINO]-[2-[N-ETHOXY-3-METHYLBUTANAMIDO]]-3-METHYL-BUTANAMIDE (ISOMER 1) and (+)-[2-[[4-[BIS-(4-CHLOROPHENYL)METHYL]-4-QUINAZOLINYL]AMINO]-[2-[N-ETHOXY-3-METHYLBUTANAMIDO]]-3-METHYL-BUTANAMIDE (ISOMER 2)

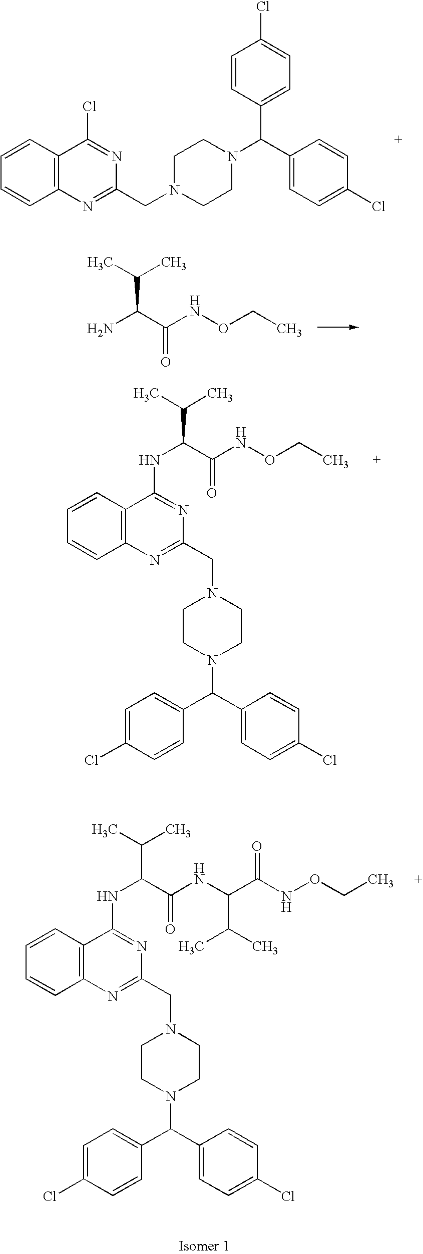

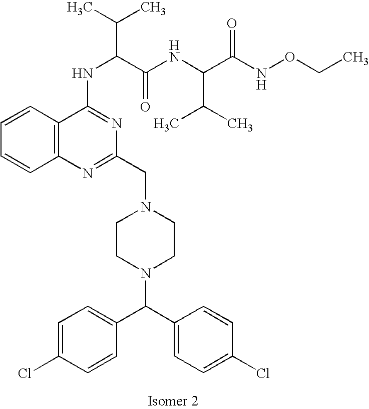

2-{4-[Bis-(4-chlorophenyl)methylpiperazin-1-ylmethyl}-4-chloroquinazoline (160 mg, 0.32 mmoles) (prepared as described in Preparative Example 8 above), (S)-(+)-amino-N-ethoxy-3-methylbutyramide (103 mg, 0.64 mmoles) and triethylamine (195.2 mg, 0.268 mL, 1.92 mmoles) were added to anhydrous 1,4-dioxane (10 mL) and the slurry was heated under argon at 102° C. for 24 h. The mixture was evaporated to dryness and the residue was taken up in dichloromethane and washed with saturated aqueous sodium bicarbonate. The organic layer was dried (MgSO₄), filtered and evaporated to dryness. The residue was purified by preparative tic on 20×20 cm 250μ silica gel plates using 5% (10% concentrated ammonium hydroxide in methanol)-dichloromethane as the eluant to give [2(S)-(−)-[[4-[bis(4-chlorophenyl)methyl]-4-quinazolinyl]amino]-N-ethoxy-3-methylbutanamide (45.2 mg, 23%): FABMS: m/z 621.8 (MH⁺); HRFABMS: m/z 621.2523 (MH⁺). Calcd. for C₃₃H₃₉Cl₂N₆O₂: m/z 621.2512; δ_{H} (CDCl₃) 1.04 (6H, d, NHCHCH(CH₃)₂), 1.23 (3H, dd, NHOCH₂CH₃), 2.44 (4H, m, N(CH₂CH₂)N), 2.71 (4H, m, N(CH₂CH₂)N), 3.64/3.74 (2H, AB system, 2CH₂N), 3.92 (3H, m, NHOCH₂CH₃), 4.21 (1H, s, NCH(C₆H₄Cl)₂), 4.28 (1H, dd, NHCHCH(CH₃)₂), 6.48 (1H, d, NHCHCH(CH₃)₂), 7.23 (4H, d, NCH(C₆H₄Cl)₂), 7.29 (4H, d, NCH(C₆H₄Cl)₂), 7.33 (1H, ddd, H₆), 7.64 (1H, ddd, H₇), 7.68 (1H, dd, H₅) and 7.72 ppm (1H, dd, H₈); δ_{C} (CDCl₃) CH₃: 13.7, 19.4, 19.7; CH₂: 65.3, 51.5, 51.5, 53.9, 53.9, 71.7; CH: 29.1, 58.8, 74.8, 121.0, 125.9, 128.5, 128.9, 128.9, 128.9, 128.9, 129.2, 129.2, 129.2, 129.2, 133.0; C: 113.4, 132.9, 132.9, 140.8, 140.8, 149.8, 159.6, 1262.2; [α]_{D}^{25° C.} −43.60 (c=0.52, MeOH), then (−)-[2-[[4-[bis-(4-chlorophenyl)methyl]-4-quinazolinyl]amino]-[2-[N-ethoxy-3-methylbutanamido]]-3-methyl-butanamide (isomer 1) (17.8 mg, 8%): FABMS: m/z 720.8 (MH⁺); HRFABMS: m/z 720.3207 (MH⁺). Calcd. for C₃₈H₄₈Cl₂N₇O₃: m/z 720.3196; δ_{H} (CDCl₃) 0.78 (3H, d, NHCHCH(CH₃)₂), 0.87 (3H, d, NHCHCH(CH₃)₂), 0.97 (3H, d, NHCHCH(CH₃)₂), 1.01 (3H, d, NHCHCH(CH₃)₂), 1.23/1.24 (3H, dd, NHOCH₂CH₃), 2.43 (4H, m, N(CH₂CH₂)N), 2.67 (4H, m, N(CH₂CH₂)N), 3.69/3.77 (2H, AB system, 2-CH₂N), 3.92 (2H, m, NHOCH₂CH₃), 4.03 (1H, dd, NHCHCH(CH₃)₂), 4.20 (1H, s, NCH(C₆H₄Cl)₂), 4.74 (1H, dd, NHCHCH(CH₃)₂), 6.32 (1H, d, NHCHCH(CH₃)₂), 7.20 (4H, d, NCH(C₆H₄Cl)₂), 7.31 (4H, d, NCH(C₆H₄Cl)₂), 7.40 (1H, ddd, H₆), 7.69 (1H, ddd, H₇), 7.74 (1H, dd, H₅) and 7.80 ppm (1H, dd, H₈); δ_{C} (CDCl₃) CH₃: 13.5, 18.6/18.9, 19.3/19.5; CH₂: 51.6, 51.6, 53.7, 53.7, 65.3, 72.3; CH: 28.9/29.4/30.2/30.7, 56.8/60.1, 74.8, 120.7, 126.0, 128.6, 128.9, 128.9, 128.9, 128.9, 129.2, 129.2, 129.2, 129.2; C: 113.4, 132.8, 132.8, 141.0, 141.0, 150.1, 159.5, 162.3, 172.3; [α]_{D}^{25° C.} −33.1° (c=0.53, MeOH) and finally (+)-[2-[[4-[bis-(4-chlorophenyl)methyl]-4-quinazolinyl]amino]-[2-[N-ethoxy-3-methylbutanamido]]-3-methyl-butanamide (isomer 2) (25.1 mg, 11%): FABMS: m/z 720.7 (MH⁺); HRFABMS: m/z 720.3204 (MH⁺). Calcd. for C₃₈H₄₈Cl₂N₇O₃: m/z 720.3196; δ_{H} (CDCl₃) 0.92 (3H, d, NHCHCH(CH₃)₂), 0.95 (3H, d, NHCHCH(CH₃)₂), 0.98 (6H, d, NHCHCH(CH₃)₂), 2.42 (4H, m, N(CH₂CH₂)N), 2.67 (4H, m, N(CH₂CH₂)N), 3.63 (2H, AB system, m, 2-CH₂N), 3.82 (2H, m, NHOCH₂CH₃), 4.10 (1H, d, NHCHCH(CH₃)₂), 4.19 (1H, s, NCH(C₆H₄Cl)₂), 4.67 (1H, d, NHCHCH(CH₃)₂), 6.38 (1H, d, NHCHCH(CH₃)₂), 7.22 (4H, d, NCH(C₆H₄Cl)₂), 7.30 (4H, d, NCH(C₆H₄Cl)₂), 7.39 (1H, ddd, H₆), 7.67 (1H, ddd, H₇), 7.76 (1H, dd, H₅) and 7.80 ppm (1H, dd, H₈); δ_{C} (CDCl₃) CH₃: 13.3, 18.5/18.9, 19.5; CH₂: 51.5, 51.5, 53.6, 53.6, 65.3, 71.9; CH: 29.4/29.9/30.7, 57.0/60.4, 74.8, 120.8, 126.0, 128.5, 128.8, 128.8, 128.8, 128.8, 129.2, 129.2, 129.2, 129.2, 132.9; C: 113.4, 132.9, 132.9, 140.8, 150.0, 159.6, 168.5, 172.7; [α]_{D}^{25° C.} +14.6° (c=0.45, MeOH).

### Example 6

### 3-METHYL-2(S)-(−)-[2-(4-PHENYLPIPERAZIN-1-YLMETHYL)QUINAZOLIN-4-YLAMINO]BUTYRAMIDE

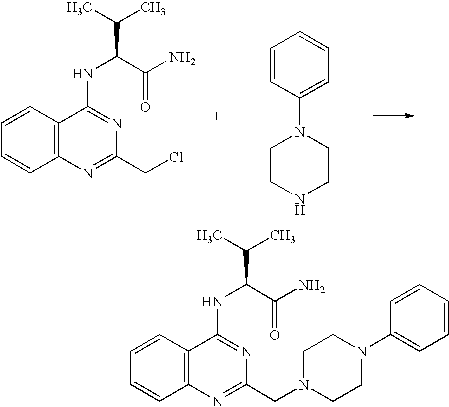

2(S)-(−)-(2-Chloromethylquinazolin-4-ylamino)-3-methylbutyramide (500 mg, 1.71 mmoles) (prepared as described in Preparative Example 12 above), 1-N-phenylpiperazine (277.1 mg, 1.71 mmoles) and anhydrous potassium carbonate (259.6 mg, 1.88 mmoles) were added to anhydrous acetonitrile (50 mL) and the mixture was heated under argon at 80° C. for 18 h. The mixture was evaporated to dryness and the residue was partitioned between dichloromethane and saturated aqueous sodium bicarbonate. The organic layer was dried (MgSO₄), filtered and evaporated to dryness. The residue was chromatographed on a silica gel column (30×2.5 cm) using 3% (10% concentrated ammonium hydroxide in methanol)-dichloromethane as the eluant to give to 3-methyl-2(S)-(−)-[2-(4-phenylpiperazin-1-ylmethyl)quinazolin-4-ylamino]butyramide (536 mg, 75%): FABMS: m/z 419.2 (MH⁺); HRFABMS: m/z 419.2561 (MH⁺). Calcd. for C₂₄H₃₁N₆O: m/z 419.2559; δ_{H} (CDCl₃) 1.04 (3H, d, NHCHCH(CH₃)₂), 1.06 (3H, d, NHCHCH(CH₃)₂), 2.38 (1H, m, NHCHCH(CH₃)₂), 2.63 (1H, bs, NH), 2.78 (4H, m, N(CH₂CH₂)N), 3.20 (4H, m, N(CH₂CH₂)N), 3.70/3.80 (2H, AB system, 2-CH₂N), 4.73 (1H, d, NHCHCH(CH₃)₂), 5.97 (1H, bs, CONH₂), 6.68 (1H, d, NHCHCH(CH₃)₂), 6.83 (1H, dd, NC₆H₅), 6.92 (2H, d, NC₆H₅), 7.12 (1H, bs, CONH₂), 7.24 (2H, dd, NC₆H₅), 7.39 (1H, ddd, H₆), 7.69 (1H, ddd, H₇) and 7.81 ppm (2H, dd, H₅ and H₈); δ_{C} (CDCl₃) CH₃: 18.9, 19.5; CH₂: 49.1, 49.1, 53.4, 53.4, 65.6; CH: 30.0, 59.8, 116.1, 116.1, 119.8, 121.0, 125.9, 128.4, 129.2, 129.2, 132.9; C: 113.6, 149.9, 151.3, 159.6, 162.6, 174.5; [α]_{D}^{25° C.} −11.3° (c=0.51, MeOH).

### Example 7

### 2(S)-(−)-[2-(4-BENZYLPIPERAZIN-1-YLMETHYL)QUINAZOLIN-4-YLAMINO]-3-METHYLBUTYRAMIDE

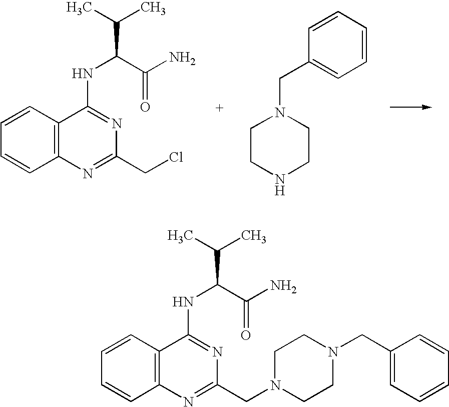

2(S)-(−)-(2-Chloromethylquinazolin-4-ylamino)-3-methylbutyramide (500 mg, 1.71 mmoles) (prepared as described in Preparative Example 12 above), 1-N-benzylpiperazine (301 mg, 0.297 mL, 1.71 mmoles) and anhydrous potassium carbonate (259.6 mg, 1.88 mmoles) were added to anhydrous acetonitrile (50 mL) and the mixture was heated under argon at 80° C. for 18 h. The mixture was evaporated to dryness and the residue was partitioned between dichloromethane and saturated aqueous sodium bicarbonate. The organic layer was dried (MgSO₄), filtered and evaporated to dryness. The residue was chromatographed on a silica gel column (30×2.5 cm) using 2% (10% concentrated ammonium hydroxide in methanol)-dichloromethane as the eluant to give 3-methyl-2(S)-(−)-[2-(4-phenylpiperazin-1-ylmethyl)quinazolin-4-ylamino]butyramide (517.1 mg, 70%): FABMS: m/z 433.3 (MH⁺); HRFABMS: m/z 433.2720 (MH⁺). Calcd. for C₂₅H₃₃N₆O: m/z 433.2716; δ_{H} (CDCl₃) 1.07 (6H, d, NHCHCH(CH₃)₂), 2.42 (1H, m, NHCHCH(CH₃)₂), 2.49 (4H, m, N(CH₂CH₂)N), 2.67 (4H, m, N(CH₂CH₂)N), 3.49 (2H, s, NCH₂C₆H₅), 3.67/3.77 (2H, AB system, 2-CH₂N), 4.74 (1H, dd, NHCHCH(CH₃)₂), 5.73 (1H, bs, CONH₂), 6.50 (1H, d, NHCHCH(CH₃)₂), 7.24 (2H, m, NCH₂C₆H₅), 7.29 (3H, m, NCH₂C₆H₅), 7.39 (1H, ddd, H₆), 7.68 (1H, ddd, H₇) and 7.77 ppm (2H, dd, H₅ and H₈); δ_{C} (CDCl₃) CH₃: 19.0, 19.5; CH₂: 53.2, 53.2, 53.2, 53.2, 63.2, 65.4; CH: 30.1, 59.7, 120.9, 125.8, 127.1, 128.3, 128.3, 128.4, 129.3, 129.3 132.8; CH: 113.6, 138.0, 150.0, 159.5, 162.8, 174.3; [α]_{D}^{25° C.} −9.8° (c=0.50, MeOH).

### Example 8

### (−)-3(R)-METHYL-2(S)-[2-(4-PHENYLPIPERAZIN-1-YLMETHYL)QUINAZOLIN-4-YLAMINO]-PENTANAMIDE

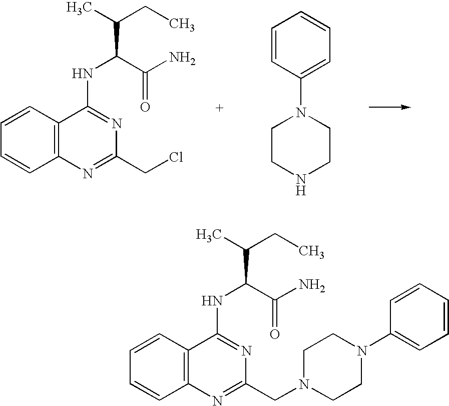

(−)-2(S)-(2-Chloromethylquinazolin-4-ylamino)-3(R)-methylpentanamide (500 mg, 1.63 mmoles) (prepared as described in Preparative Example 13 above), 1-N-phenylpiperazine (264.4 mg, 0.249 mL, 1.63 mmoles) and anhydrous potassium carbonate (247.8 mg, 1.8 mmoles) were added to anhydrous acetonitrile (50 mL) and the mixture was heated under nitrogen at 80° C. for 18 h. The mixture was evaporated to dryness and partitioned between dichloromethane and saturated aqueous sodium bicarbonate and the organic layer was dried (MgSO₄), filtered and evaporated to dryness. The residue was chromatographed on a silica gel column (30×2.5 cm) using 1-2% (10% concentrated ammonium hydroxide in methanol)-dichloromethane as the eluant to give (−)-3(R)-methyl-2(S)-[2-(4-phenylpiperazin-1-ylmethyl)quinazolin-4-ylamino]pentanamide (414 mg, 59%): FABMS: m/z 433.3 (MH⁺); HRFABMS: m/z 433.2721 (MH⁺). Calcd. for C₂₅H₃₃N₆O: m/z 433.2716; δ_{H} (CDCl₃) 0.89 (3H, t, NHCHCH(CH₃)CH₂CH₃), 1.02 (3H, d, NHCHCH(CH₃)CH₂CH₃), 1.24 (1H, m, NHCHCH(CH₃)CH₂CH₃), 1.64 (1H, m, NHCHCH(CH₃)CH₂CH₃), 2.08 (1H, m, NHCHCH(CH₃)CH₂CH₃), 2.31 (2H, bs, CONH₂), 2.77 (4H, m, N(CH₂CH₂)N), 3.23 (4H, m, N(CH₂CH₂)N), 3.71/3.82 (2H, AB system, 2-CH₂N), 4.98 (1H, m, NHCHCH(CH₃)CH₂CH₃), 5.78 (1H, bs, NHCHCH(CH₃)CH₂CH₃), 6.86 (1H, m, NC₆H₅), 6.92 (2H, d, NC₆H₅), 7.23 (2H, d, NC₆H₅), 7.40 (1H, ddd, H₆), 7.69 (1H, ddd, H₇), 7.77 (1H, dd, H₅) and 7.84 ppm (1H, dd, H₈); δ_{C} (CDCl₃) CH₃: 10.8, 15.6; CH₂: 25.3, 48.7, 48.7, 53.3, 53.3, 65.6; CH: 36.2, 57.7, 116.1, 116.1, 120.0, 121.2, 126.0, 127.9, 129.2, 129.2, 132.9; C: 113.7, 149.7, 151.2, 159.8, 162.2, 175.0; [α]_{D}^{25° C.} −13.3° (c=0.51, MeOH).

### Example 9

### (−)-2(S)-(2-{4-[BIS-(4-CHLOROPHENYL)METHYL]PIPERAZIN-1-YLMETHYL}QUINAZOLIN-4-YLAMINO)-3(R)-METHYLPENTANAMIDE

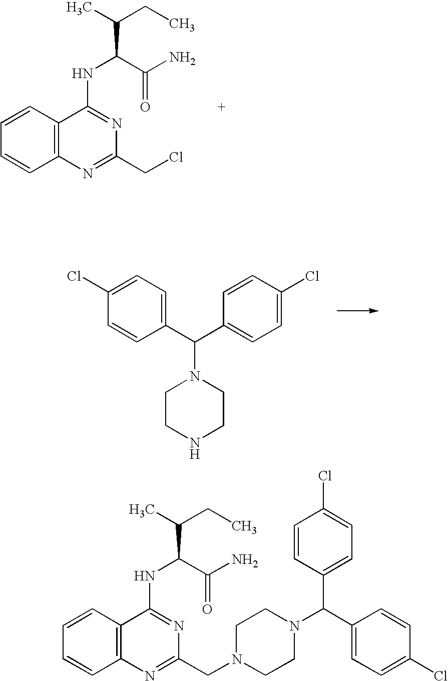

(−)-2(S)-(2-Chloromethylquinazolin-4-ylamino)-3(R)-methylpentanamide (500 mg, 1.63 mmoles) (prepared as described in Preparative Example 13 above), 1-(4,4′-dichlorobenzhydryl)piperazine (523.6 mg, 1.63 mmoles) (prepared as described in Preparative Example 1) and anhydrous potassium carbonate (247.8 mg, 1.8 mmoles) were added to anhydrous acetonitrile (50 mL) and the mixture was heated under nitrogen at 80° C. for 18 h. The mixture was evaporated to dryness and partitioned between dichloromethane and saturated aqueous sodium bicarbonate and the organic layer was dried (MgSO₄), filtered and evaporated to dryness. The residue was chromatographed on a silica gel column (30×2.5 cm) using 1% (10% concentrated ammonium hydroxide in methanol)-dichloromethane as the eluant to give (−)-2(S)-(2-{4-[bis-(4-chlorophenyl)methyl]piperazin-1-ylmethyl}quinazolin-4-ylamino)-3(R)-methylpentanamide (621.4 mg, 64%): ESMS: m/z 591.1 (MH⁺); HRFABMS: m/z 591.2401 (MH⁺). Calcd. for C₃₂H₃₇Cl₂N₆O: m/z 591.2406; δ_{H} (CDCl₃) 0.88 (3H, m, NHCHCH(CH₃)CH₂CH₃), 1.02 (3H, m, NHCHCH(CH₃)CH₂CH₃), 1.27 (1H, m, NHCHCH(CH₃)CH₂CH₃), 1.66 (1H, m, NHCHCH(CH₃)CH₂CH₃), 2.17 (1H, m, NHCHCH(CH₃)CH₂CH₃), 2.40 (4H, m, N(CH₂CH₂)N), 2.64 (4H, m, N(CH₂CH₂)N), 3.69/3.76 (2H, AB system, 2-CH₂N), 4.18 (1H, s, NCH(C₆H₄Cl)₂), 4.80 (1H, m, NHCHCH(CH₃)CH₂CH₃), 5.31 (1H, bs, CONH₂), 5.79 (1H, bs, NHCHCH(CH₃)CH₂CH₃), 6.53 (1H, bs, CONH₂), 7.06-7.37 (8H, m, NCH(C₆H₄Cl)₂), 7.40 (1H, m, H₆), 7.69 (1H, m, H₇), 7.69 (1H, dd, H₅) and 7.77 ppm (1H, dd, H₈); δ_{C} (CDCl₃) CH₃: 11.1, 15.7; CH₂: 25.4, 51.8, 51.8, 53.6, 53.6, 65.4; CH: 36.0, 58.4, 74.8, 120.9, 125.9, 128.4, 128.9, 128.9, 128.9, 128.9, 129.1, 129.1, 129.1, 129.1, 132.9; C: 113.6, 132.9, 132.9, 140.9, 140.9, 149.9, 159.4, 162.6; [α]_{D}^{25° C.} −12.6° (c=0.52, MeOH).

### Example 10

### 2-(2-{4-[BIS-(4-CHLOROPHENYL)METHYL]PIPERAZIN-1-YLMETHYL}QUINAZOLIN-4-YLAMINO)-4-METHYLPENTANOIC ACID AMIDE

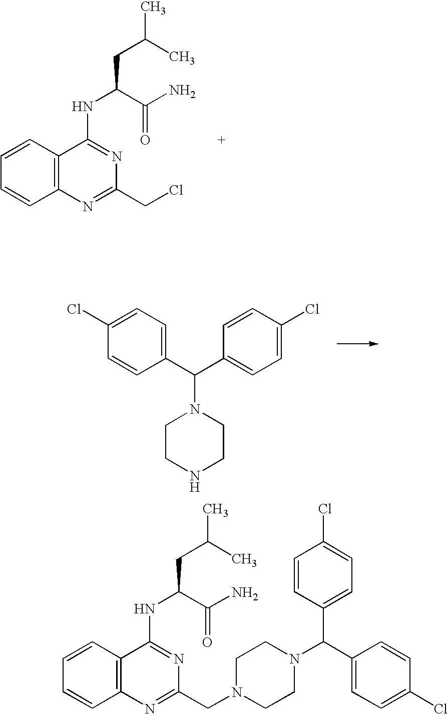

(+)-2(S)-(2-Chloromethylquinazolin-4-ylamino)-3(R)-4-methylpentanamide (prepared as described in Preparative Example 13 above), 1-(4,4′-dichlorobenzhydryl)piperazine (prepared as described in Preparative Example 1) and anhydrous potassium carbonate may be reacted under essentially the same conditions as those described in Example 9 to give the title compound.

### Example 11

### (+)-N,N-DIMETHYL-N′-[2-{4-[PHENYL-(4-TRIFLUOROMETHOXYPHENYL)METHYL]-PIPERAZIN-1-YLMETHYL}QUINAZOLIN-4-YL]PROPANE-1,3-DIAMINE (ISOMER 1) and (−)-N,N-DIMETHYL-N′-[2-{4-[PHENYL-(4-TRIFLUOROMETHOXYPHENYL)METHYL]-PIPERAZIN-1-YLMETHYL}QUINAZOLIN-4-YL]PROPANE-1,3-DIAMINE (ISOMER 2)

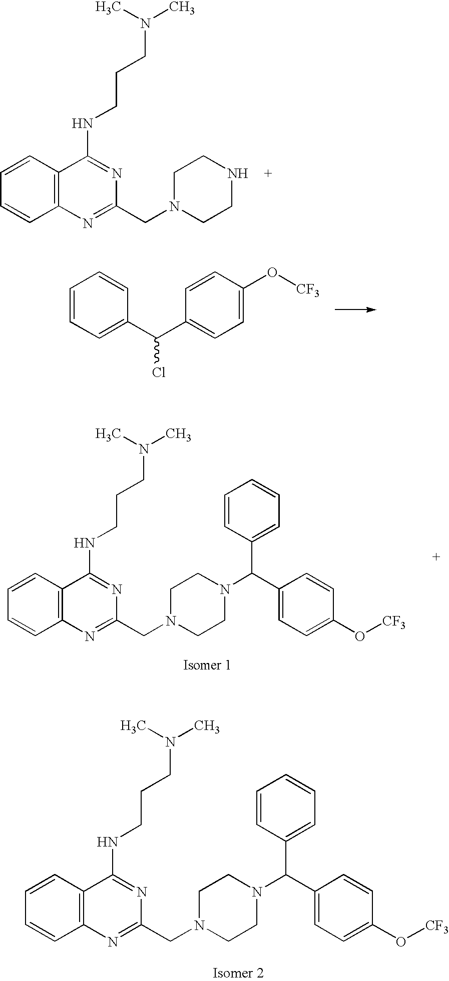

N,N-Dimethyl-N′-(2-piperazin-1-ylmethylquinazolin-4-yl)propane-1,3-diamine (283.8 mg, 0.864 mmoles) (prepared as described in Preparative Example 24, step C above), 4-trifluoromethoxybenzhydryl chloride (510.3 mg, 1.73 mmoles) (prepared as described in Preparative Example 5 above), anhydrous potassium carbonate (119.4 mg, 0.864 mmoles) and anhydrous potassium iodide (143 mg, 0.864 mmoles) were added to anhydrous acetonitrile (4 mL) and the mixture was stirred at 25° C. for 41 h. The mixture was filtered and the solids were washed with anhydrous acetonitrile (2×20 mL). The combined filtrates were evaporated to dryness and the residue was chromatographed on a silica gel column (60×2.5 cm) using 5% (10% concentrated ammonium hydroxide in methanol)-dichloromethane as the eluant to give (±)-N,N-dimethyl-N′-[2-{4-[phenyl-(4-trifluoromethoxyphenyl)methyl]-piperazin-1-ylmethyl}quinazolin-4-yl]propane-1,3-diamine (167.4 mg, 33%).

The racemate (165 mg) was subjected to chiral HPLC on a Chiralpak® AD column using hexane:isopropyl alcohol:diethylamine: 90:10:0.2 to give in the order of elution (+)-N,N-dimethyl-N′-[2-{4-[phenyl-(4-trifluoromethoxyphenyl)methyl]-piperazin-1-ylmethyl}quinazolin-4-yl]propane-1,3-diamine (Isomer 1) (71.7 mg, 14%): FABMS: m/z 579.4 (MH⁺); HRFABMS: m/z 579.3066 (MH⁺). Calcd. for C₃₂H₃₈F₃N₆O: m/z 579.3059; δ_{H} (CDCl₃) 1.84 (2H, m, NHCH₂CH₂CH₂N(CH₃)₂), 2.36 NHCH₂CH₂CH₂N(CH₃)₂), 2.47 (4H, m, N(CH₂CH₂)N), 2.57 (2H, m, NHCH₂CH₂CH₂N(CH₃)₂), 2.77 (4H, m, N(CH₂CH₂)N), 3.73 (2H, m, NHCH₂CH₂CH₂N(CH₃)₂), 3.75 (2H, s, 2-CH₂N), 4.26 (1H, s, NCH(C₆H₅)(C₆H₄OCF₃)), 7.10 (2H, d, C₆H₄OCF₃), 7.19 (1H, dd, C₆H₅), 7.27 (2H, dd, C₆H₅), 7.38 (1H, ddd, H₆), 7.40 (2H, d, C₆H₄OCF₃), 7.44 (2H, d, C₆H₅), 7.56 (1H, dd, H₅), 7.65 (1H, ddd, H₇), 7.78 (1H, dd, H₈) and 8.63 ppm (1H, bs, NHCHCH(CH₃)CH₂CH₃); δ_{C} (CDCl₃) CH₃: 45.6, 45.6; CH₂: 24.7, 42.5, 51.9, 51.9, 53.8, 53.8, 59.9, 65.4; CH: 75.6, 120.9, 120.9, 120.9, 125.2, 127.1, 128.0, 128.0, 128.3, 128.6, 128.6, 129.2, 129.2, 132.0; C: 114.1, 114.1, 141.8, 142.4, 148.0, 149.9, 159.8, 163.5; [α]_{D}^{25° C.} +6.3° (c=0.34, MeOH) and (−)-N,N-dimethyl-N′-[2-{4-[phenyl-(4-trifluoromethoxyphenyl)methyl]-piperazin-1-ylmethyl}quinazolin-4-yl]propane-1,3-diamine (Isomer 2) (70.6 mg, 14%): FABMS: m/z 579.4 (MH⁺); HRFABMS: m/z 579.3066 (MH⁺). Calcd. for C₃₂H₃₈F₃N₆O: m/z 579.3059; δ_{H} (CDCl₃) 1.84 (2H, m, NHCH₂CH₂CH₂N(CH₃)₂), 2.36 NHCH₂CH₂CH₂N(CH₃)₂), 2.49 (4H, m, N(CH₂CH₂)N), 2.59 (2H, m, NHCH₂CH₂CH₂N(CH₃)₂), 2.77 (4H, m, N(CH₂CH₂)N), 3.73 (2H, m, NHCH₂CH₂CH₂N(CH₃)₂), 3.75 (2H, 2, 2-CH₂N), 4.26 (1H, s, NCH(C₆H₅)(C₆H₄OCF₃)), 7.10 (2H, d, C₆H₄OCF₃), 7.19 (1H, dd, C₆H₅), 7.27 (2H, dd, C₆H₅), 7.38 (1H, ddd, H₆), 7.39 (2H, d, C₆H₄OCF₃), 7.43 (2H, d, C₆H₅), 7.56 (1H, dd, H₅), 7.64 (1H, ddd, H₇), 7.78 (1H, dd, H₈) and 8.63 ppm (1H, bs, NHCHCH(CH₃)CH₂CH₃); δ_{C} (CDCl₃) CH₃: 45.5, 45.5; CH₂: 24.7, 42.5, 51.9, 51.9, 53.8, 53.8, 59.9, 65.4; CH: 75.6, 120.9, 120.9, 120.9, 125.2, 127.1, 128.0, 128.0, 128.3, 128.6, 128.6, 129.2, 129.2, 132.0; C: 114.1, 114.1, 141.8, 142.4, 148.0, 149.9, 159.8, 163.5; [α]_{D}^{25° C.} −8.3° (c=0.32, MeOH).

Both isomer 1 and 2 were found to have % Residual T @ 2 ug/mL ratings according to scintillation proximity assay (SPA) of “B” and EC50 ratings according to the proliferation assay (MB468) of “A”. (See descriptions of assays below).

### Example 12

### (+)-N′-(2-{4-[(5-BROMOPYRIDIN-2-YL)-(3,5-DICHLOROPHENYL)METHYL]PIPERAZIN-1-YLMETHYL}QUINAZOLIN-4-YL)-N,N-DIMETHYLPROPANE-1,3-DIAMINE (ISOMER 1) and (−)-N′-(2-{4-[(5-BROMOPYRIDIN-2-YL)-(3,5-DICHLOROPHENYL)METHYL]PIPERAZIN-1-YLMETHYL}QUINAZOLIN-4-YL)-N,N-DIMETHYLPROPANE-1,3-DIAMINE (ISOMER 2)

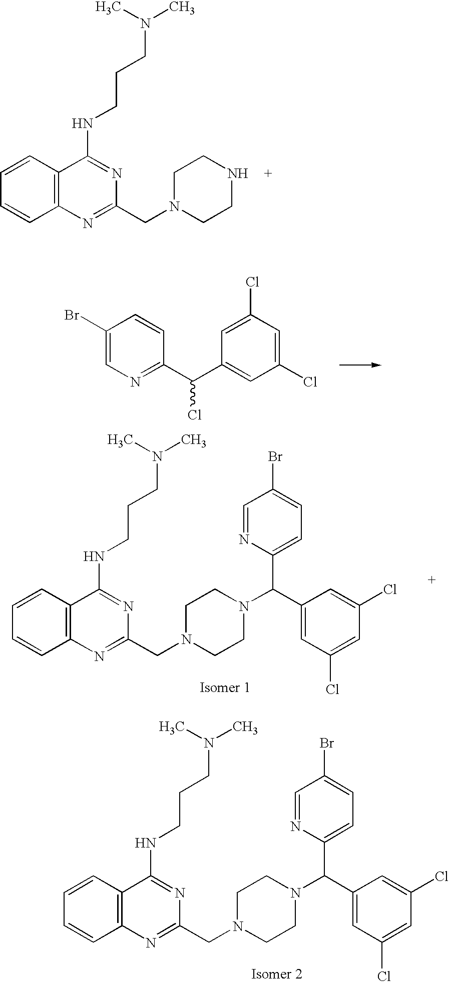

N,N-Dimethyl-N′-(2-piperazin-1-ylmethylquinazolin-4-yl)propane-1,3-diamine (334 mg, 1.02 mmoles) (prepared as described in Preparative Example 24, Step C above), was dissolved in anhydrous acetonitrile (4 mL) and 5-bromo-2-[chloro-(3,5-dichlorophenyl)methyl]pyridine (530.2 mg, 1.52 mmoles) (prepared as described in Preparative Example 4, Step D above) dissolved in anhydrous acetonitrile (6 mL) was added. Anhydrous potassium carbonate (154.8 mg, 1.12 mmoles) and anhydrous potassium iodide (169.2 mg, 1.02 mmoles) were added and the mixture was stirred at 25° C. for 44 h. The mixture was filtered and the solids were washed with anhydrous acetonitrile and dichloromethane. The combined filtrates were evaporated to dryness and the residue was chromatographed on a silica gel column (60×2.5 cm) using 4% (10% concentrated ammonium hydroxide in methanol)-dichloromethane as the eluant to give (±)-N′-(2-{4-[(5-bromopyridin-2-yl)-(3,5-dichlorophenyl)methyl]piperazin-1-ylmethyl}quinazolin-4-yl)-N,N-dimethylpropane-1,3-diamine (295.6 mg, 45%): FABMS: m/z 642.1 (MH⁺).

The racemate (395.6 mg) was subjected to chiral HPLC on a Chiralpak® AD column using hexane:isopropyl alcohol:diethylamine: 95:5:0.2 to give in the order of elution (+)-N′-(2-{4-[(5-bromopyridin-2-yl)-(3,5-dichlorophenyl)methyl]piperazin-1-ylmethyl}quinazolin-4-yl)-N,N-dimethylpropane-1,3-diamine (Isomer 1) (142.5 mg): FABMS: m/z 642.1 (MH⁺); HRFABMS: m/z 643.1530 (Isotope MH⁺). Calcd. for C₃₁H₃₆BrCl₂N₆: m/z 643.1541; δ_{H} (CDCl₃) 1.83 (2H, m, NHCH₂CH₂CH₂N(CH₃)₂), 2.36 (6H, s, NHCH₂CH₂CH₂N(CH₃)₂), 2.50 (4H, m, N(CH₂CH₂)N), 2.57 (2H, m, NHCH₂CH₂CH₂N(CH₃)₂), 2.77 (4H, m, N(CH₂CH₂)N), 3.73 (2H, m, NHCH₂CH₂CH₂N(CH₃)₂), 3.75 (2H, s, 2-CH₂N), 4.36 (1H, s, NCH(C₅H₃BrN)(C₆H₃Cl₂)), 7.18 (1H, dd, C₆H₃Cl₂), 7.35 (2H, d, C₆H₃Cl₂), 7.37 (1H, ddd, H₆), 7.41 (1H, dd, C₅H₃BrN), 7.56 (1H, d, H₆), 7.64 (1H, ddd, H₇), 7.76 (1H, dd, H₈), 7.76 (1H, dd, C₅H₃BrN), 8.56 (1H, d, C₅H₃BrN) and 8.62 ppm (1H, bs, NHCH₂CH₂CH₂N(CH₃)₂); δ_{C} (CDCl₃) CH₃: 45.5, 45.5; CH₂: 24.7, 42.5, 51.8, 51.8, 53.4, 53.4, 59.8, 65.2; CH: 76.4, 121.0, 123.7, 125.3, 126.7, 126.7, 127.7, 128.3, 132.1, 139.6, 150.6; C: 114.1, 119.4, 135.1, 135.1, 144.3, 149.8, 159.4, 159.8, 163.3; [α]_{D}^{25° C.} +4.9° (c=0.33, MeOH) and (−)-N′-(2-{4-[(5-bromopyridin-2-yl)-(3,5-dichlorophenyl)methyl]piperazin-1-ylmethyl}quinazolin-4-yl)-N,N-dimethylpropane -1,3-diamine (Isomer 2) (148.4 mg): FABMS: m/z 642.1 (MH⁺); HRFABMS: m/z 643.1530 (Isotope MH⁺). Calcd. for C₃₁H₃₆BrCl₂N₆: m/z 643.1541; δ_{H} (CDCl₃) 1.83 (2H, m, NHCH₂CH₂CH₂N(CH₃)₂), 2.37 (6H, s, NHCH₂CH₂CH₂N(CH₃)₂), 2.50 (4H, m, N(CH₂CH₂)N), 2.58 (2H, m, NHCH₂CH₂CH₂N(CH₃)₂), 2.78 (4H, m, N(CH₂CH₂)N), 3.73 (2H, m, NHCH₂CH₂CH₂N(CH₃)₂), 3.75 (2H, s, 2-CH₂N), 4.36 (1H, s, NCH(C₅H₃BrN)(C₆H₃Cl₂)), 7.17 (1H, dd, C₆H₃Cl₂), 7.33 (2H, d, C₆H₃Cl₂), 7.37 (1H, ddd, H₆), 7.42 (1H, dd, C₅H₃BrN), 7.55 (1H, d, H₆), 7.64 (1H, ddd, H₇), 7.76 (1H, dd, H₈), 7.77 (1H, dd, C₅H₃BrN), 8.56 (1H, d, C₅H₃BrN) and 8.64 ppm (1H, bs, NHCH₂CH₂CH₂N(CH₃)₂); δ_{C} (CDCl₃) CH₃: 45.6, 45.6; CH₂: 24.7, 42.5, 51.8, 51.8, 53.4, 53.4, 59.9, 65.2; CH: 76.4, 120.9, 123.7, 125.3, 126.7, 126.7, 127.7, 128.3, 132.1, 139.6, 150.6; C: 114.1, 119.4, 135.1, 135.1, 144.3, 149.8, 159.4, 159.9, 163.3; [α]_{D}^{25° C.} −2.7° (c=0.31, MeOH) and (±)-N′-(2-{4-[(5-bromopyridin-2-yl)-(3,5-dichlorophenyl)methyl]piperazin -1-ylmethyl}quinazolin-4-yl)-N,N-dimethylpropane-1,3-diamine (80 mg).

### Example 13

### (+)-N′-{2-[4-(3-BROMO-8-CHLORO-6,11-DIHYDRO-5H-BENZO[5,6]CYCLOHEPTA[1,2-b]PYRIDIN-11-YL)PIPERAZIN-1-YLMETHYL]QUINAZOLIN-4-YL}-N,N-DIMETHYLPROPANE-1,3-DIAMINE (ISOMER 1) and (−)-N′-{2-[4-(3-BROMO-8-CHLORO-6,11-DIHYDRO-5H-BENZO[5,6]CYCLOHEPTA[1,2-b]PYRIDIN-11-YL)PIPERAZIN-1-YLMETHYL]QUINAZOLIN-4-YL}-N,N-DIMETHYLPROPANE-1,3-DIAMINE (ISOMER 2)

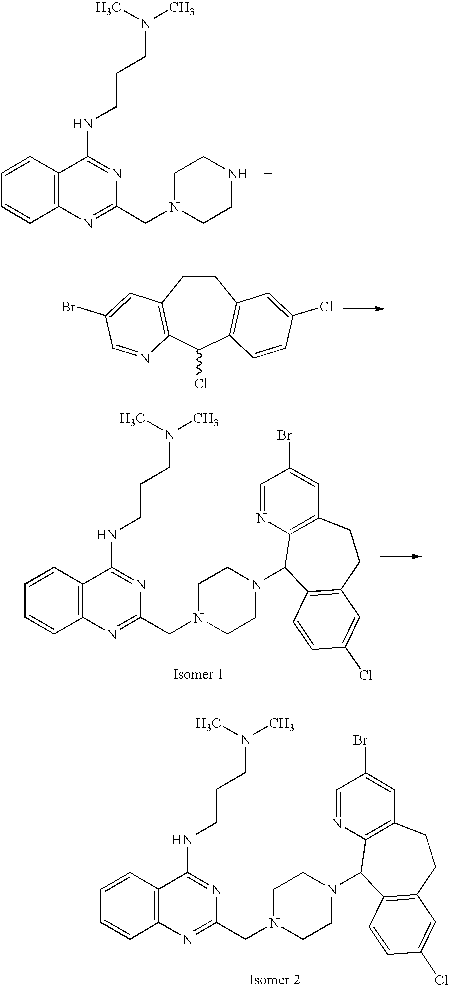

N,N-Dimethyl-N′-(2-piperazin-1-ylmethylquinazolin-4-yl)propane-1,3-diamine (200 mg, 0.61 mmoles) (prepared as described in Preparative Example 24, Step C above) and triethylamine (0.255 mL, 1.83 mmoles) were dissolved in anhydrous dichloromethane (2 mL) and 3-bromo-8,11-dichloro-6,11-dihydro[5,6]cyclohepta[1,2-b]pyridine (312.2 mg, 0.91 mmoles) (prepared from the alcohol as described in Preparative Example 40 in U.S. Pat. No. 5,719,148; Feb. 17, 1998) dissolved in anhydrous dichloromethane (5 mL) was added. The mixture was stirred at 25° C. for 20 h. and then evaporated to dryness. The residue was taken up in dichloromethane (10 mL) and 10% conc. ammonium hydroxide in methanol (10 mL) was added. The mixture was evaporated to dryness and the residue was chromatographed on a silica gel column (60×2.5 cm) using 6% (10% concentrated ammonium hydroxide in methanol)-dichloromethane as the eluant to give (±)-N′-{2-[4-(3-bromo-8-chloro-6,11-dihydro-5H-benzo[5,6]cyclohepta[1,2-b]pyridin -11-yl)piperazin-1-ylmethyl]quinazolin-4-yl}-N,N-dimethylpropane-1,3-diamine (260 mg, 67%): FABMS: m/z 634.2 (MH⁺).

The racemate (255 mg) was subjected to chiral HPLC on a Chiralpak® AD column using hexane:isopropyl alcohol:diethylamine: 90:10:0.2 to give in the order of elution (+)-N′-{2-[4-(3-bromo-8-chloro-6,11-dihydro-5H-benzo[5,6]cyclohepta[1,2-b]pyridin-11-yl)piperazin-1-ylmethyl]quinazolin-4-yl}-N,N-dimethylpropane-1,3-diamine (118.4 mg, 46%): FABMS: m/z 634.3 (MH⁺); HRFABMS: m/z 636.2051 (MH⁺ isotope peak). Calcd. for C₃₂H₃₈BrClN₇: m/z 636.2042; δ_{H} (CDCl₃) 1.81 (2H, m, NHCH₂CH₂CH₂N(CH₃)₂), 2.32 (6H, s, NHCH₂CH₂CH₂N(CH₃)₂), 2.58 (2H, m, NHCH₂CH₂CH₂N(CH₃)₂), 3.70 (2H, m, NHCH₂CH₂CH₂N(CH₃)₂), 3.70 (2H, s, 2-CH₂N), 4.34 (1H, s, H_{11′″}), 7.06 (2H, m, H_{7′″} and H_{9′″}), 7.18 (1H, d, H₅), 7.36 (1H, ddd, H₆), 7.54 (1H, d, H_{9′″}), 7.54 (1H, d, H_{4′″}), 7.64 (1H, ddd, H₇), 7.76 (1H, dd, H₈), 8.34 (1H, d, H_{2′″}) and 8.64 ppm (1H, bs, NHCH₂CH₂CH₂N(CH₃)₂); δ_{C} (CDCl₃) CH₃: 45.5, 45.5; CH₂: 24.6, 30.3, 30.7, 42.5, 51.7, 51.7, 53.7, 53.7, 59.9, 65.3; CH: 79.4, 120.9, 125.2, 126.1, 128.3, 130.5, 132.1, 132.5, 141.0, 146.9; C: 114.1, 119.7, 133.8, 136.2, 136.8, 140.7, 149.8, 157.0, 159.8, 163.5; [α]_{D}^{25° C.} +14.1° (c=0.39, MeOH) and (−)-N′-{2-[4-(3-bromo-8-chloro-6,11-dihydro-5H-benzo[5,6]cyclohepta[1,2-b]pyridin-11-yl)piperazin-1-ylmethyl]quinazolin-4-yl}-N,N-dimethylpropane-1,3-diamine (124.5 mg, 49%): FABMS: m/z 634.3 (MH⁺); HRFABMS: m/z 636.2051 (MH⁺ isotope peak). Calcd. for C₃₂H₃₈BrClN₇: m/z 636.2042; δ_{H} (CDCl₃) 1.83 (2H, m, NHCH₂CH₂CH₂N(CH₃)₂), 2.34 (6H, s, NHCH₂CH₂CH₂N(CH₃)₂), 2.58 (2H, m, NHCH₂CH₂CH₂N(CH₃)₂), 3.70 (2H, s, 2-CH₂N), 3.73 (2H, m, NHCH₂CH₂CH₂N(CH₃)₂), 4.34 (1H, S, H_{11′″}), 7.06 (2H, m, H_{7′″} and H_{9′″}), 7.18 (1H, d, H₅), 7.36 (1H, ddd, H₆), 7.54 (1H, d, H_{9′″}), 7.54 (1H, d, H_{4′″}), 7.63 (1H, ddd, H₇), 7.76 (1H, dd, H₈), 8.34 (1H, d, H_{2′″}) and 8.63 ppm (1H, bs, NHCH₂CH₂CH₂N(CH₃)₂); δ_{C} (CDCl₃) CH₃: 45.5, 45.5; CH₂: 24.6, 30.3, 30.7, 42.5, 51.7, 51.7, 53.7, 53.7, 59.8, 65.3; CH: 79.4, 120.9, 125.2, 126.1, 128.2, 130.5, 132.1, 132.5, 141.0, 146.9; C: 114.1, 119.7, 133.8, 136.2, 136.8, 140.7, 149.8, 156.9, 159.8, 163.5; [α]_{D}^{25° C.} −12.7° (c=0.38, MeOH).

### Example 14

### N′-{2-[4-(10,11-DIHYDRO-5H-DIBENZO[a,d]CYCLOHEPTEN-5-YL)PIPERAZIN-1-YLMETHYL]QUINAZOLIN-4-yl}-N,N-DIMETHYLPROPANE -1,3-DIAMINE

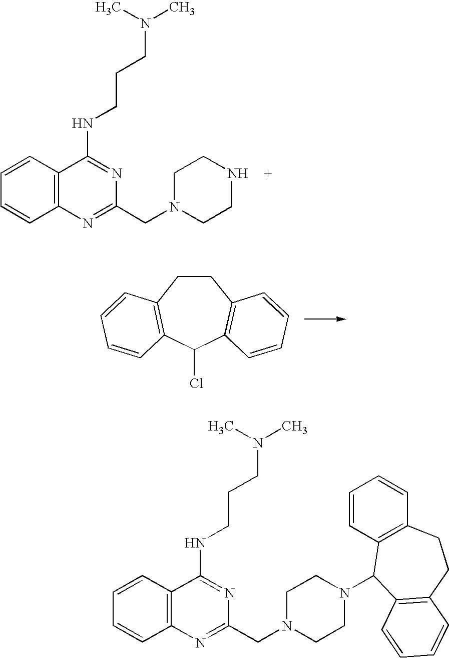

N,N-Dimethyl-N′-(2-piperazin-1-ylmethylquinazolin-4-yl)propane-1,3-diamine (200 mg, 0.61 mmoles) (prepared as described in Preparative Example 24, Step C above) and triethylamine (0.255 mL, 1.83 mmoles) were dissolved in anhydrous dichloromethane (5 mL) and 5-chloro-10,11-dihydro-5H-dibenzo[a,d]cycloheptene (5-chlorodibenzosuberane) (209 mg, 0.91 mmoles) was added. The mixture was stirred at 25° C. for 20 h. and then evaporated to dryness. The residue was chromatographed on a silica gel column (30×2.5 cm) using 5% (10% concentrated ammonium hydroxide in methanol)-dichloromethane as the eluant to give N′-{2-[4-(10,11-dihydro-5H-dibenzo[a,d]cyclohepten-5-yl)piperazin-1-ylmethyl]quinazolin-4-yl}-N,N-dimethylpropane-1,3-diamine (216.1 mg, 68%): FABMS: m/z 521.4 (MH⁺); HRFABMS: m/z 521.3392 (MH⁺). Calcd. for C₃₃H₄₁N₆: m/z 521.3393; δ_{H} (CDCl₃) 1.93 (2H, m, NHCH₂CH₂CH₂N(CH₃)₂), 2.40 (4H, m, N(CH₂CH₂)N), 2.43 (6H, s, NHCH₂CH₂CH₂N(CH₃)₂), 2.70 (4H, m, N(CH₂CH₂)N), 2.77 (2H, m, NHCH₂CH₂CH₂N(CH₃)₂), 3.75 (2H, m, NHCH₂CH₂CH₂N(CH₃)₂), 3.75 (2H, s, 2-CH₂N), 4.00 (1H, s, H_{11′″}), 7.00-7.20 (8H, m, Ar—H), 7.38 (1H, ddd, H₆), 7.64 (1H, ddd, H₇), 7.74 (1H, dd, H₅), 7.77 (1H, dd, H₈) and 8.57 ppm (1H, bs, NHCH₂CH₂CH₂N(CH₃)₂); δ_{C} (CDCl₃) CH₃: 44.9, 44.9; CH₂: 24.4, 31.8, 31.8, 41.2, 51.7, 51.7, 53.8, 53.8, 58.7, 65.0; CH: 79.1, 121.4, 125.4, 125.4, 125.4, 127.6, 127.6, 128.1, 130.7, 130.7, 130.8, 130.8, 132.2; C: 114.0, 139.4, 139.4, 139.7, 139.7, 149.7, 159.8, 162.9.

### Example 15

### N1-[2-[[4-[BIS-(4-CHLOROPHENYL)METHYL]-2(S)-(2-METHYLPROPYL)-1-PIPERAZINYL]METHYL]-4-QUINAZOLINYL)-N 3,N3-DIMETHYL-1,3-PROPANEDIAMINE

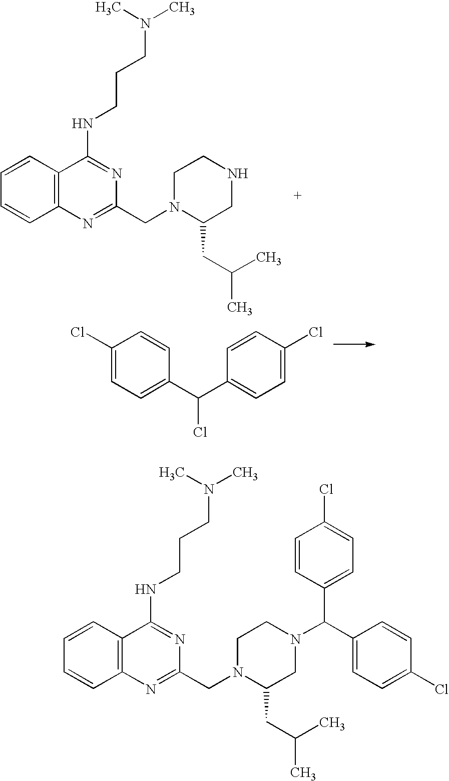

N′-[2-{4-Benzyl-2(S)-(+)-isobutylpiperazin-1-ylmethyl}quinazolin-4-yl]-N,N-dimethylpropane-1,3-diamine (137.8 mg, 0.29 mmoles) (prepared as described in Preparative Example 25, Step G above) (20 mg, 0.052 mmoles), bis-(4-chlorophenyl)methyl chloride [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)] (28.3 mg, 0.104 mmoles), anhydrous potassium carbonate (7.2 mg, 0.052 mmoles) and anhydrous potassium iodide (8.6 mg, 0.052 mmoles) were added to anhydrous acetonitrile (2 mL) and anhydrous dichloromethane (0.5 mL) and the mixture was stirred at 25° C. for 165 h. The mixture was filtered and the solids were rinsed with anhydrous acetonitrile and dichloromethane. The combined filtrates were evaporated to dryness and the residue was subjected to preparative tic on a 250□ silica gel plate (20×20 cm) using 30% methanol in dichloromethane as the eluant to give N1-[2-[[4-[bis-(4-chlorophenyl)methyl]-2(S)-(2-methyl propyl)-1-piperazinyl]methyl]-4-quinazolinyl)-N3,N3-dimethyl-1,3-propanediamine (2 mg, 6%): ESMS: m/z 619.4 (MH⁺).

### Example 16

### [3-(2-{4-[BIS-(4-CHLOROPHENYL)METHYL]PIPERAZIN-1-YLMETHYL}QUINAZOLIN-4-YLOXY)PROPYL]DIMETHYLAMINE

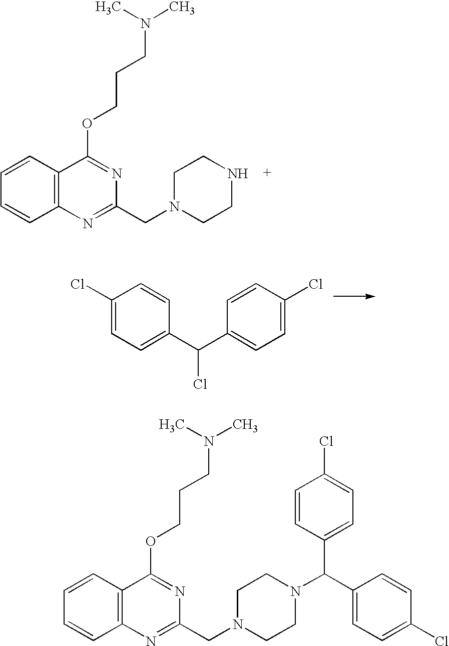

Dimethyl-[3-(2-piperazin-1-ylmethylquinazolin-4-yloxy)propyl]amine (140 mg, 0.425 mmoles) (prepared as described in Preparative Example 26, Step B above), bis-(4-chlorophenyl)methyl chloride [prepared as described in: S. Younes, G. Baziard-Mouysset, G. de Saqui-Sannes, J. L. Stigliani, M. Payard, R. Bonnafous and J. Tisne-Versailles, Eur. J. Med. Chem., 28, 943-948 (1993)] (231 mg, 0.85 mmoles), anhydrous potassium carbonate (58.7 mg, 0.425 mmoles) and anhydrous potassium iodide (70.5 mg, 0.425 mmoles) were added to anhydrous acetonitrile (4 mL) and the mixture was stirred at 25° C. for 20 h. The mixture was filtered and the solids were rinsed with anhydrous acetonitrile. The combined filtrates were evaporated to dryness and the residue was chromatographed on a silica gel column (30×2.5 cm) using 5% (10% concentrated ammonium hydroxide in methanol)-dichloromethane as the eluant to give [3-(2-{4-[bis-(4-chlorophenyl)methyl]piperazin-1-ylmethyl}quinazolin-4-yloxy)propyl]dimethylamine (34.6 mg) and unreacted dimethyl-[3-(2-piperazin-1-ylmethylquinazolin-4-yloxy)propyl]amine (79.2 mg, 33%). The latter was recycled as described above only using a reaction time of 45 h to give a total yield of [3-(2-{4-[bis-(4-chlorophenyl)methyl]piperazin-1-ylmethyl}quinazolin-4-yloxy)propyl]dimethylamine (92.9 mg, 57%): FABMS: m/z 564.3 (MH⁺); HRFABMS: m/z 564.2288 (MH⁺). Calcd. for C₃₁H₃₆Cl₂N₅O: m/z 564.2297; δ_{H} (CDCl₃) 2.07 (2H, dt, OCH₂CH₂CH₂N(CH₃)₂), 2.29 (6H, s, OCH₂CH₂CH₂N(CH₃)₂), 2.46 (4H, bs, NCH₂CH₂N), 2.54 (2H, dd, OCH₂CH₂CH₂N(CH₃)₂), 2.73 (4H, bs, NCH₂CH₂N), 3.84 (2H, s, 2-CH₂N), 4.21 (1H s, NCH(C₆H₄Cl)₂), 4.62 (2H, dd, OCH₂CH₂CH₂N(CH₃)₂), 7.23 (4H, d, NCH(C₆H₄Cl)₂), 7.30 (4H, d, NCH(C₆H₄Cl)₂), 7.50 (1H, ddd, H₆), 7.78 (1H, ddd, H₇), 7.91 (1H, dd, H₅) and 8.13 ppm (1H, dd, H₈); δ_{C} (CDCl₃) CH₃: 45.5, 45.5; CH₂: 27.0, 51.8, 51.8, 53.6, 53.6, 56.5, 65.0, 65.3; CH: 74.8, 123.4, 126.5, 127.7, 128.8, 128.8, 128.8, 128.8, 129.2, 129.2, 129.2, 129.2, 133.4; C: 115.2, 132.8, 132.8, 141.0, 141.0, 151.3, 162.7, 166.7. The compound was found to have % Residual T @ 2 ug/mL rating according to the scintillation proximity assay (SPA) of “D” and an EC50 rating according to the proliferation assay of “D” (See descriptions of assays below).

### Example 17

### Reaction of 3-METHYL-2(S)-(−)-[2-(PIPERAZIN-1-YLMETHYL)QUINAZOLIN-4-YLAMINO]BUTYRIC ACID METHYL ESTER with an ALDEHYDE/KETONE LIBRARY

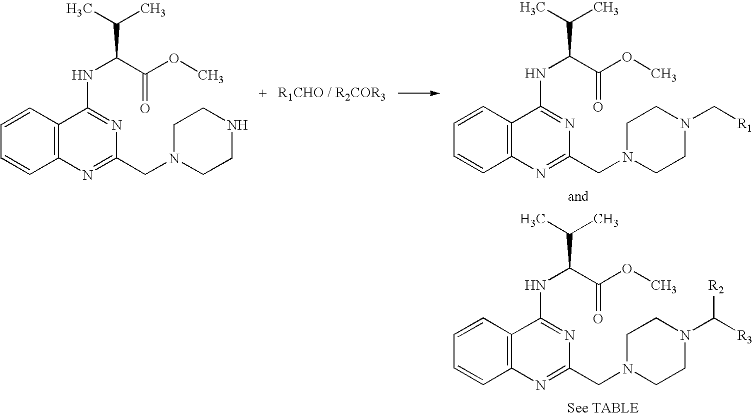

A stock solution of 3-methyl-2(S)-(−)-[2-(piperazin-1-ylmethyl)quinazolin-4-ylamino]butyric acid methyl ester (1 mL, 0.0279 mmoles) (prepared as described in Preparative Example 21 above) in THF was added to the tubes in four 24 tube Bohdan Miniblocks. A 1M stock solution of each of the individual aldehydes R₁CHO and ketones (R₂COR₃) in THF (00.1 mL, 0.1 mmoles) was then added, one to each tube. Sodium triacetoxyborohydride (18 mg, 0.0865 mmoles) was added to each tube, followed by additional THF (0.5 mL). The Miniblocks were sealed and shaken at 25° C. for 20 h. Methanol (0.5 mL) was added to each tube. MP-TsOH resin (˜0.12 g) was added to each tube and the blocks were shaken at 25° C. for 4 h. The tubes were drained and the resin was washed three times with methanol, shaking for 5 min each time, to remove unreacted reagents. Ammonia in methanol (2N, 2 mL) was added to each tube and the blocks were again shaken at 25° C. for 20 min. The methanol filtrates were collected and the resin was again shaken with ammonia in methanol (2N, 2 mL). The combined filtrates from each tube were evaporated to dryness overnight, on a Speedvac concentrator. The resulting samples were analyzed by LCMS and any samples that were <70% pure, were further purified by preparative LCMS. The compounds that were prepared having a purity >70%, are listed in the table below.

### Examples 17-1 Through 17-96

| EXAMPLE | STRUCTURE | MW | LCMS m/z |
|---|---|---|---|
| 17-3 | 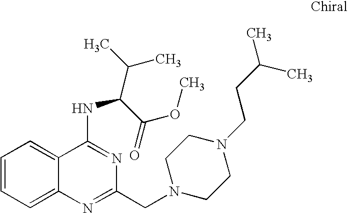 | 427.6 | 428.2 |
| 17-2 | 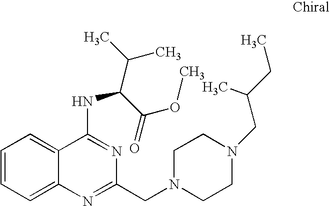 | 427.6 | 428.2 |
| 17-3 | 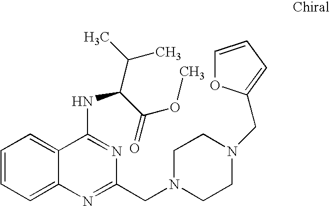 | 437.6 | 438.2 |
| 17-4 | 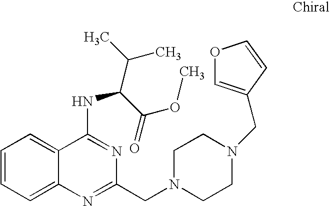 | 437.6 | 438.2 |
| 17-5 | 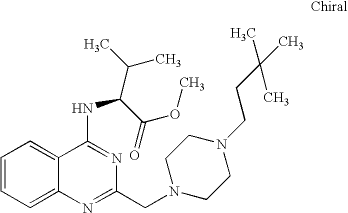 | 441.6 | 442.2 |
| 17-6 | 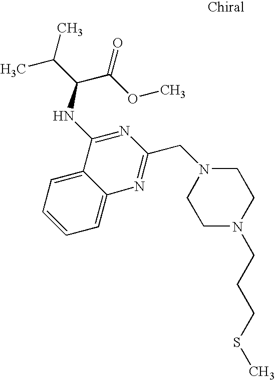 | 445.6 | 446.2 |
| 17-7 | 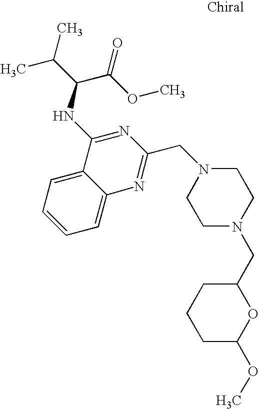 | 485.6 | 486.3 |
| 17-8 | 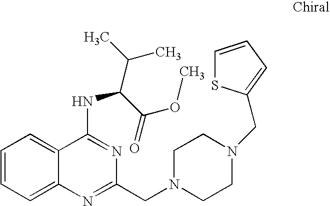 | 453.6 | 454.2 |
| 17-9 | 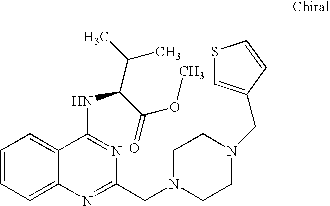 | 453.6 | 454.2 |
| 17-10 | 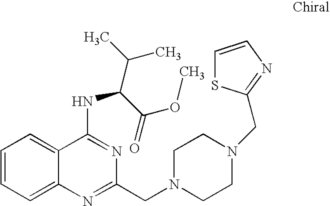 | 454.6 | 454.3 |
| 17-11 | 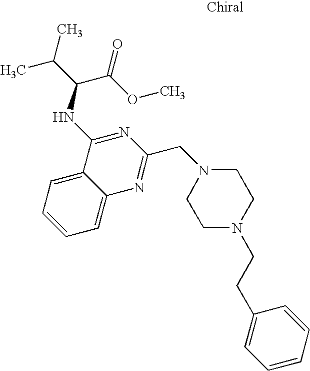 | 461.6 | 462.3 |
| 17-12 | 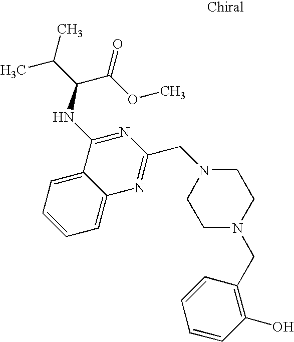 | 463.6 | 464.3 |
| 17-13 | 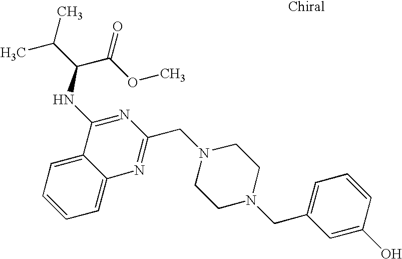 | 463.6 | 464.3 |
| 17-14 | 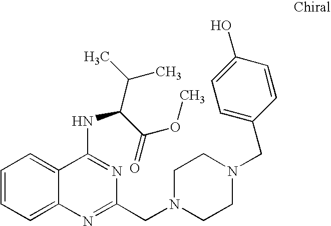 | 463.6 | 464.3 |
| 17-15 | 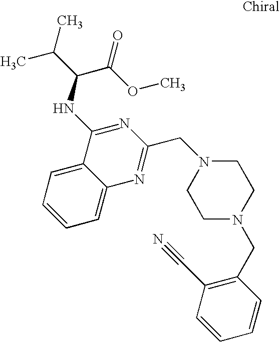 | 472.6 | 473.3 |
| 17-16 | 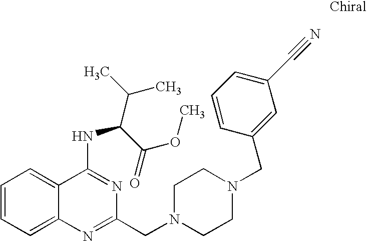 | 472.6 | 473.3 |
| 17-17 | 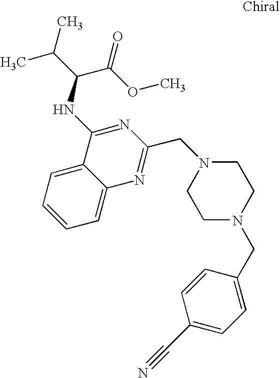 | 472.6 | 473.3 |
| 17-18 | 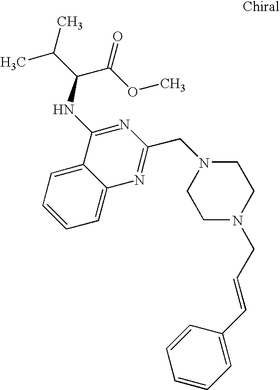 | 473.6 | 474.3 |
| 17-19 | 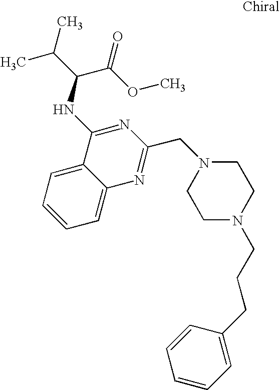 | 475.6 | 476.3 |
| 17-20 | 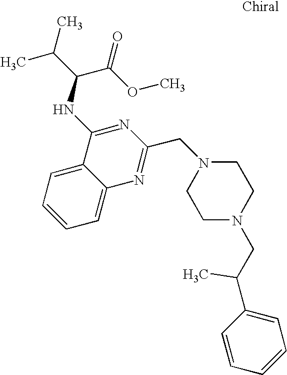 | 475.6 | 476.3 |
| 17-21 | 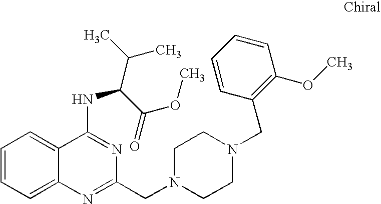 | 477.6 | 478.3 |
| 17-22 | 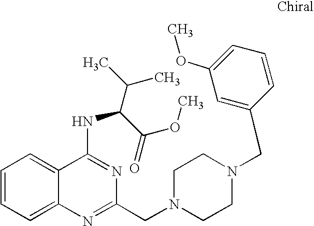 | 477.6 | 478.3 |
| 17-23 | 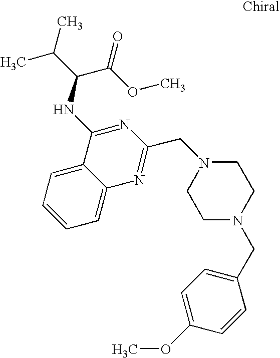 | 477.6 | 478.3 |
| 17-24 | 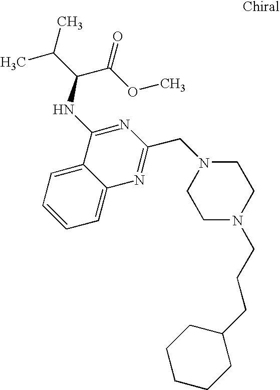 | 481.7 | 482.3 |
| 17-25 | 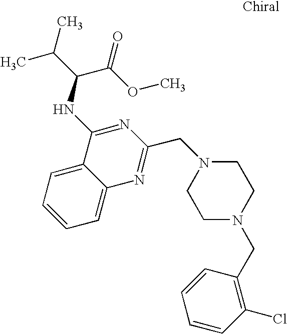 | 482.0 | 482.3 |
| 17-26 | 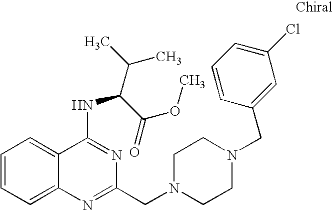 | 482.0 | 482.3 |
| 17-27 | 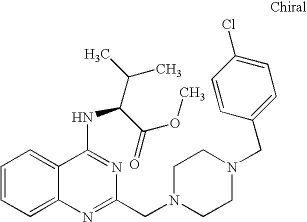 | 482.0 | 482.3 |
| 17-28 | 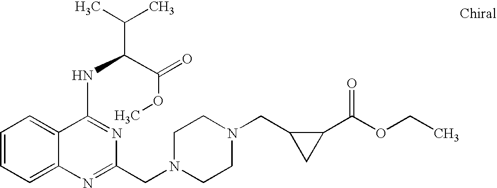 | 483.6 | 484.3 |
| 17-29 | 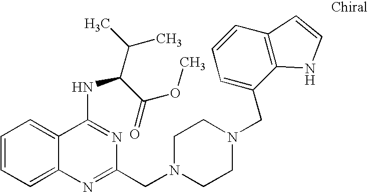 | 486.6 | 487.3 |
| 17-30 | 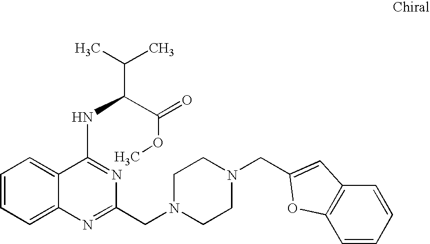 | 487.6 | 487.6 |
| 17-31 | 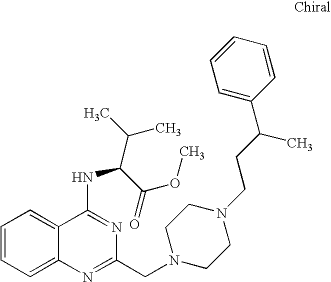 | 489.7 | 490.3 |
| 17-32 | 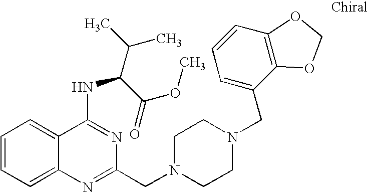 | 491.6 | 492.3 |
| 17-33 | 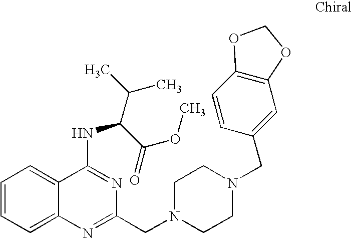 | 491.6 | 492.3 |
| 17-34 | 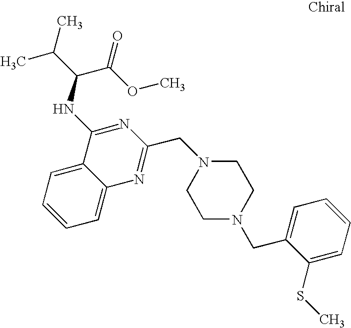 | 493.7 | 494.3 |
| 17-35 | 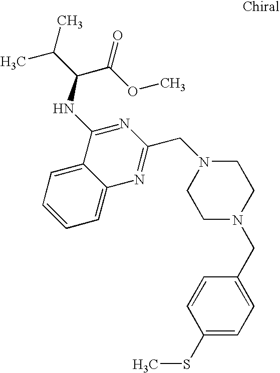 | 493.7 | 494.3 |
| 17-36 | 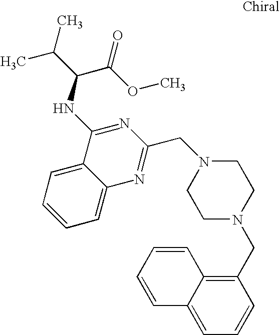 | 497.7 | 498.3 |
| 17-37 | 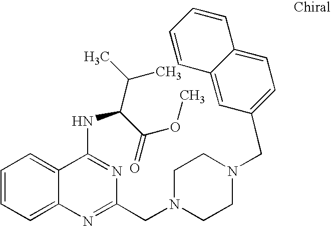 | 497.7 | 498.3 |
| 17-38 | 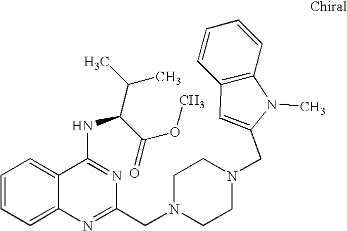 | 500.7 | 501.3 |
| 17-39 | 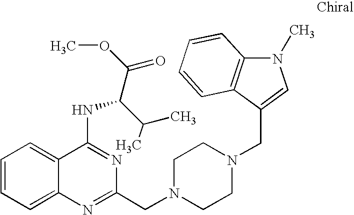 | 500.7 | 501.3 |
| 17-40 | 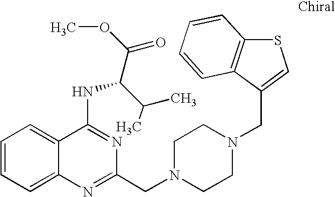 | 503.7 | 504.3 |
| 17-41 | 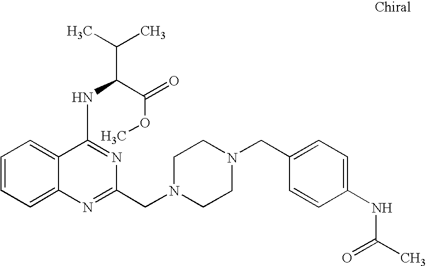 | 504.6 | 505.3 |
| 17-42 | 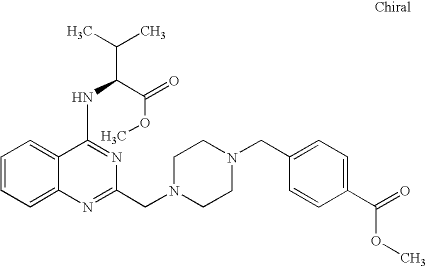 | 505.6 | 506.3 |
| 17-43 | 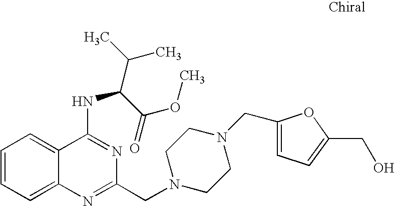 | 467.6 | 468.3 |
| 17-44 | 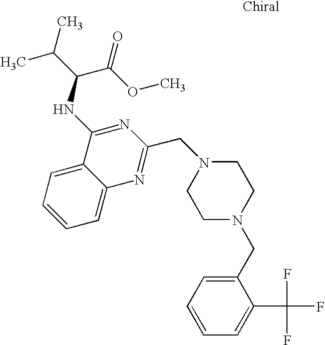 | 515.6 | 516.3 |
| 17-45 | 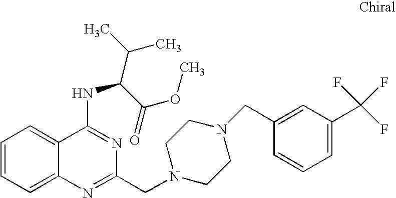 | 515.6 | 516.3 |
| 17-46 | 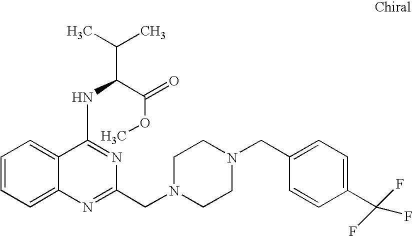 | 515.6 | 516.3 |
| 17-47 | 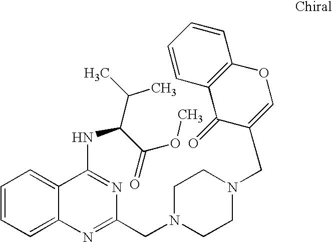 | 515.6 | 516.3 |
| 17-48 | 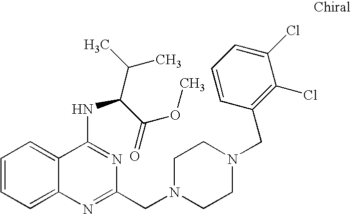 | 516.5 | 516.3 |
| 17-49 | 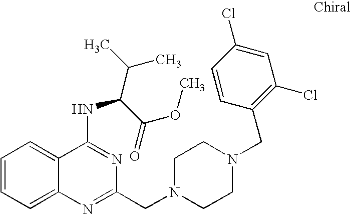 | 516.5 | 516.3 |
| 17-50 | 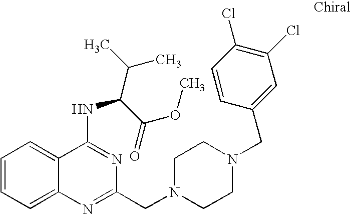 | 516.5 | 516.3 |
| 17-51 | 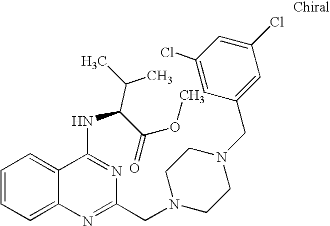 | 516.5 | 516.3 |
| 17-52 | 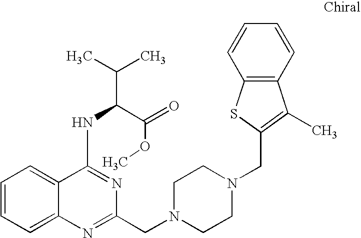 | 517.7 | 518.3 |
| 17-53 | 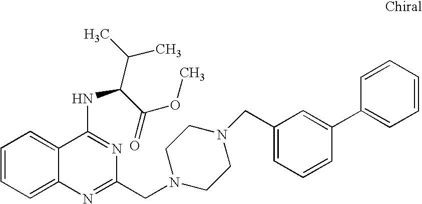 | 523.7 | 524.3 |
| 17-54 | 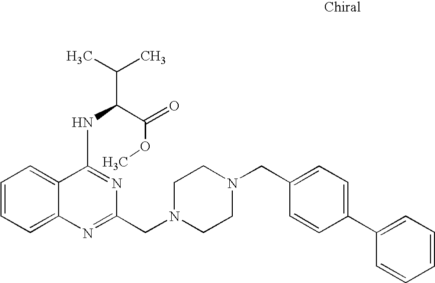 | 523.7 | 524.3 |
| 17-55 | 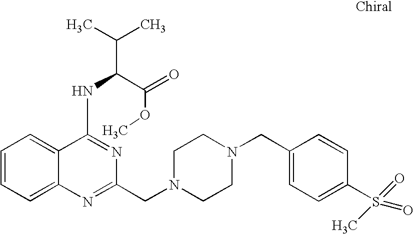 | 525.7 | 526.3 |
| 17-56 | 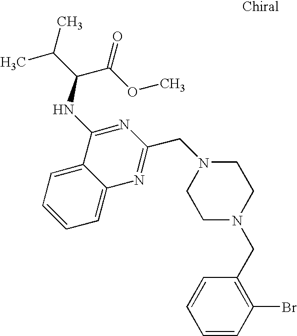 | 526.5 | 528,.3 |
| 17-57 | 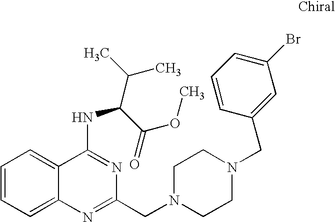 | 526.5 | 528.3 |
| 17-58 | 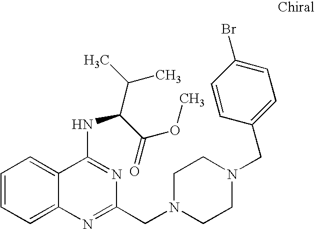 | 526.5 | 528.3 |
| 17-59 | 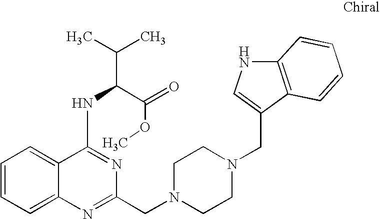 | 486.6 | 487.3 |
| 17-60 | 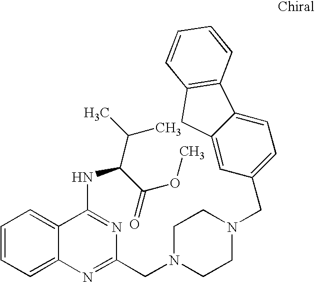 | 535.7 | 536.3 |
| 17-61 | 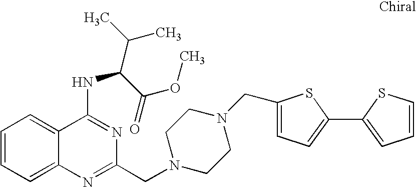 | 535.7 | 536.3 |
| 17-62 | 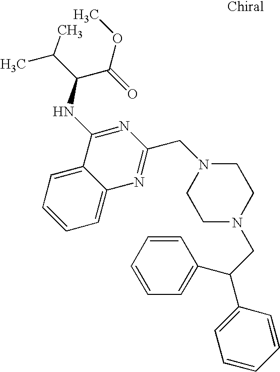 | 537.7 | 538.3 |
| 17-63 | 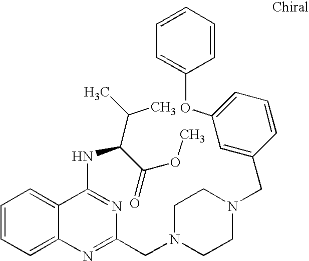 | 539.7 | 540.3 |
| 17-64 | 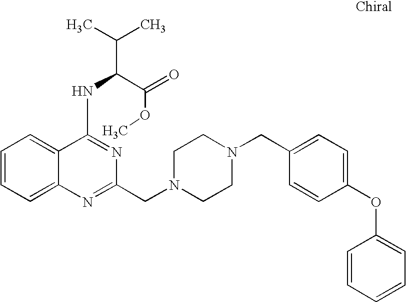 | 539.7 | 540.3 |
| 17-65 | 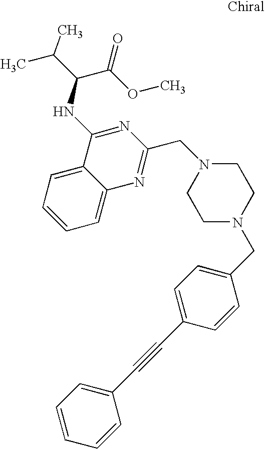 | 547.7 | 548.3 |
| 17-66 | 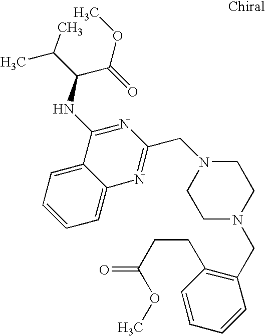 | 535.6 | 536.3 |
| 17-67 | 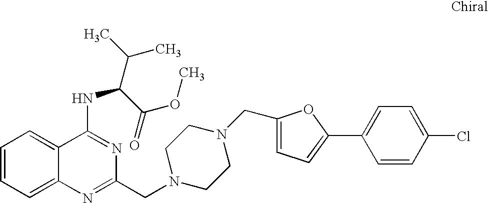 | 548.1 | 548.3 |
| 17-68 | 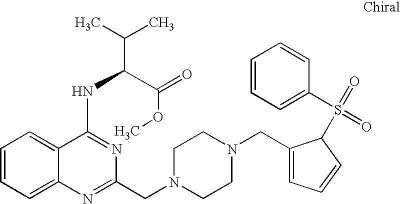 | 576.7 | 577.1 |
| 17-69 | 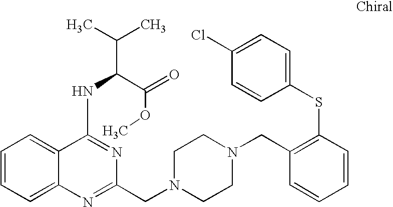 | 590.2 | 590.3 |
| 17-70 | 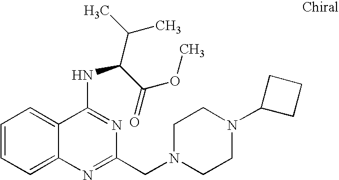 | 411.6 | 412.2 |
| 17-71 | 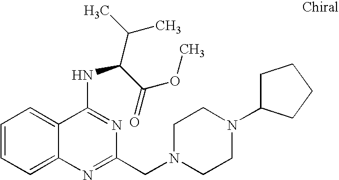 | 425.6 | 426.2 |
| 17-72 | 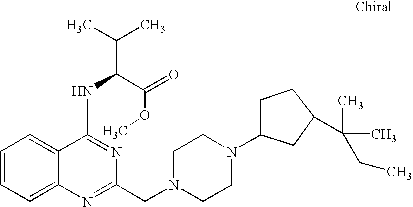 | 495.7 | 496.3 |
| 17-73 | 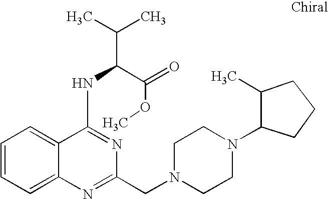 | 439.6 | 440.2 |
| 17-74 | 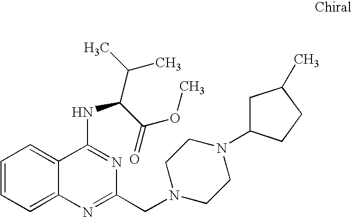 | 439.6 | 440.2 |
| 17-75 | 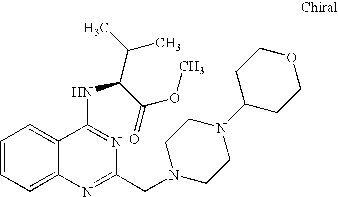 | 441.6 | 442.2 |
| 17-76 | 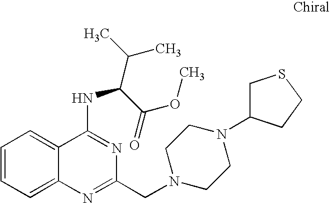 | 443.6 | 444.2 |
| 17-77 | 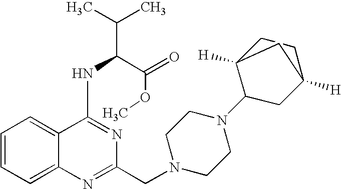 | 451.6 | 452.2 |
| 17-78 | 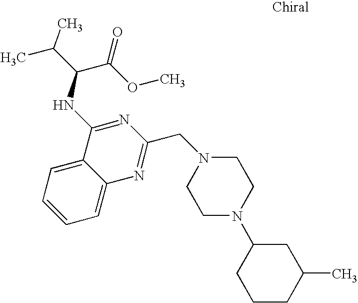 | 453.6 | 454.2 |
| 17-79 | 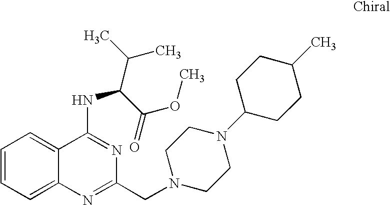 | 453.6 | 454.2 |
| 17-80 | 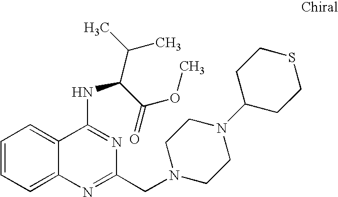 | 457.6 | 458.3 |
| 17-81 | 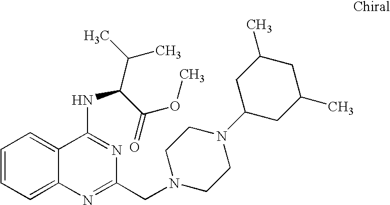 | 467.7 | 468.3 |
| 17-82 | 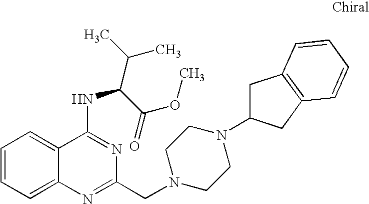 | 473.6 | 747.3 |
| 17-83 | 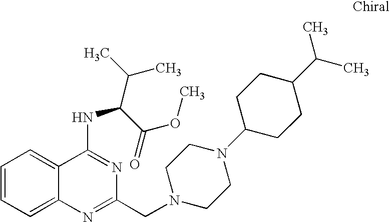 | 481.7 | 482.3 |
| 17-84 | 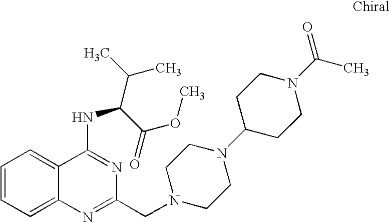 | 482.6 | 483.3 |
| 17-85 | 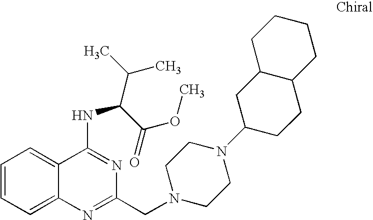 | 493.7 | 494.3 |
| 17-86 | 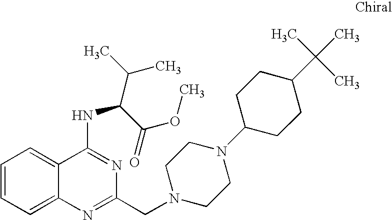 | 495.7 | 496.3 |
| 17-87 | 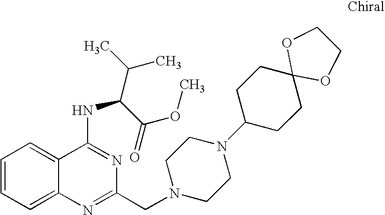 | 497.6 | 498.3 |
| 17-88 | 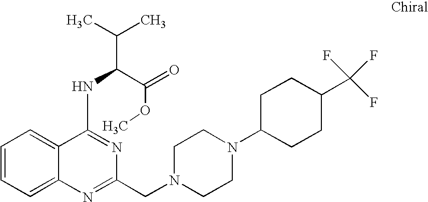 | 507.6 | 508.3 |
| 17-89 | 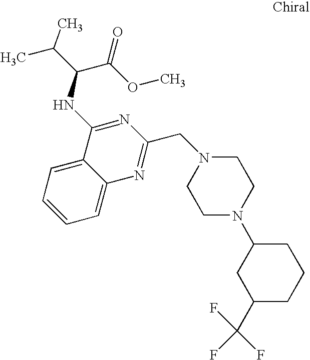 | 507.6 | 508.3 |
| 17-90 | 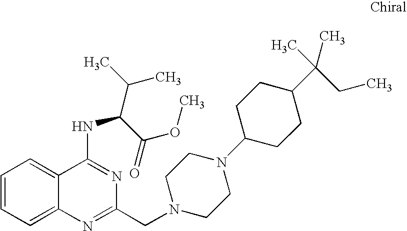 | 509.7 | 510.3 |
| 17-91 | 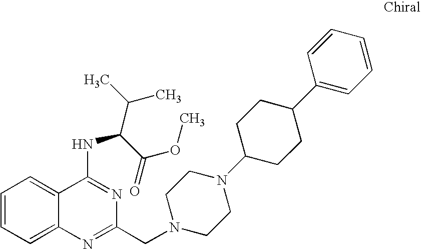 | 515.7 | 516.3 |
| 17-92 | 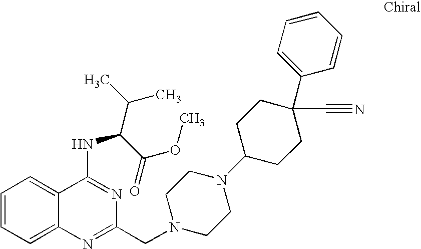 | 540.7 | 541.3 |
| 17-93 | 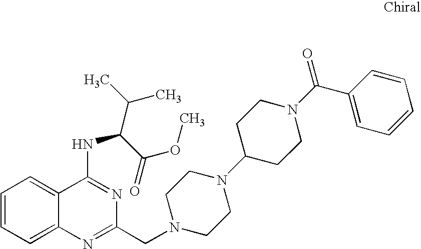 | 544.7 | 545.3 |
| 17-94 | 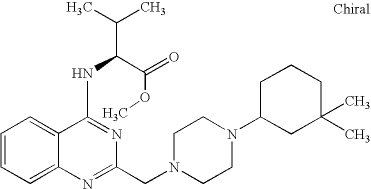 | 467.7 | 468.3 |
| 17-95 | 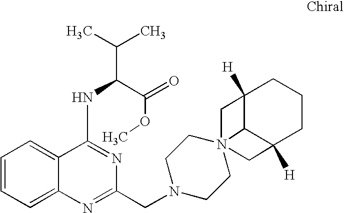 | 479.7 | 480.3 |

### Example 18

### Reaction of 3-METHYL-2(S)-(−)-[2-(PIPERAZIN-1-YLMETHYL)QUINAZOLIN-4-YLAMINO]BUTYRIC ACID METHYL ESTER with an ACID LIBRARY

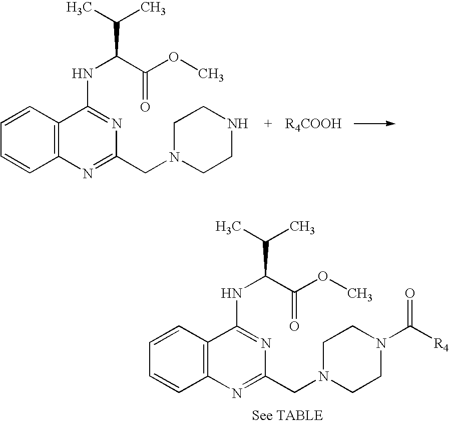

A stock solution of 3-methyl-2(S)-(−)-[2-(piperazin-1-ylmethyl)quinazolin-4-ylamino]butyric acid methyl ester (1 mL, 0.0233 mmoles) (prepared as described in Preparative Example 21 above) in THF was added to the tubes in three 24 tube Bohdan Miniblocks. PS-EDC resin (41 mg, 0.0583 mmoles) was added to each tube, followed by a stock solution of HOBT in THF (0.5 mL, 0.0350 mmoles). 1M stock solutions of each of the individual acids (0.03 mL, 0.0303 mmoles) were added, one to each tube, and the Miniblocks were sealed and shaken at 25° C. for 21 h. PS-Isocyanate resin (3 equivalents, 0.0699 mmoles) was added to each tube, followed by PS-Trisamine resin (6 equivalents, 0.1398 mmoles) and the Miniblocks were shaken at 25° C. for 4 h. The tubes were drained and the resin was washed with THF (2×1 mL), shaking for 5 min each time. The filtrates from each tube were combined and evaporated to dryness overnight, on a Speedvac concentrator. The resulting samples were evaluated by LCMS and those that were >70% pure are listed in the table below.

### Examples 18-1 Through 18-72

| EXAMPLE | STRUCTURE | MW | LCMS M+ |
|---|---|---|---|
| 18-1 | 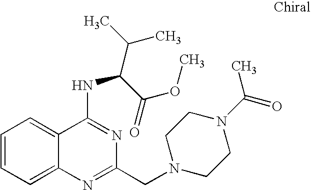 | 399.5 | 400.1 |
| 18-2 | 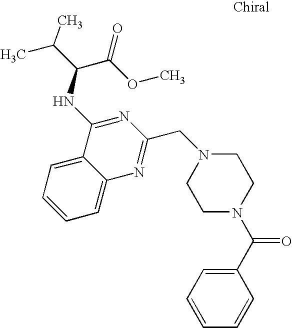 | 461.6 | 462.1 |
| 18-3 | 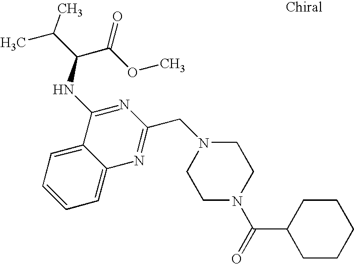 | 467.6 | 468.1 |
| 18-4 | 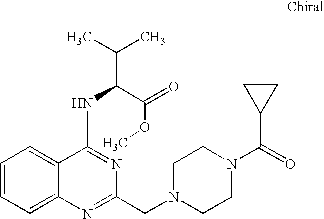 | 425.5 | 426.2 |
| 18-5 | 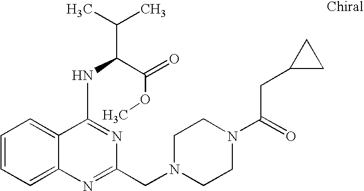 | 439.6 | 440.2 |
| 18-6 | 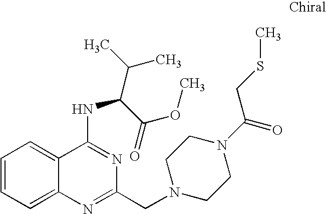 | 445.6 | 446.2 |
| 18-7 | 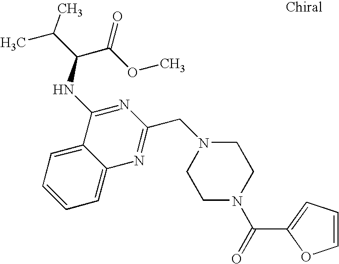 | 451.5 | 452.2 |
| 18-8 | 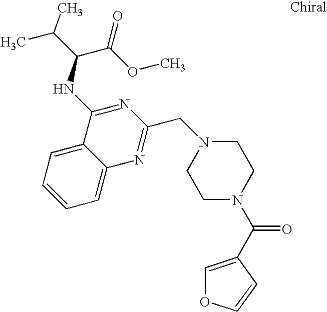 | 451.5 | 452.2 |
| 18-9 | 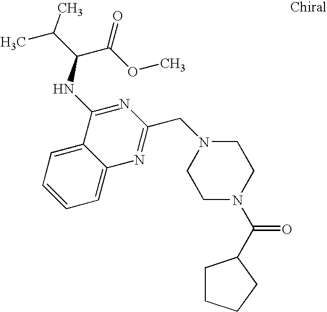 | 453.6 | 454.2 |
| 18-10 | 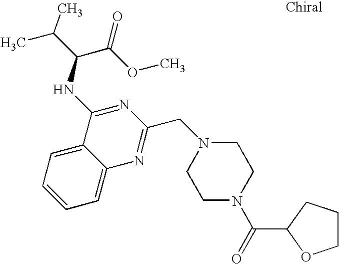 | 455.6 | 456.3 |
| 18-11 | 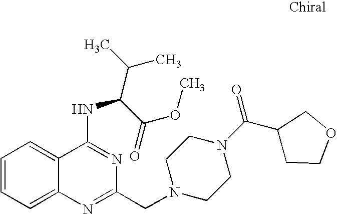 | 455.6 | 456.3 |
| 18-12 | 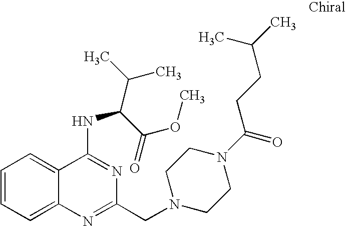 | 455.6 | 456.3 |
| 18-13 | 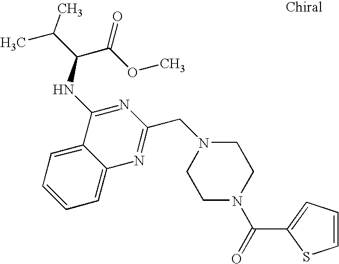 | 467.6 | 468.3 |
| 18-14 | 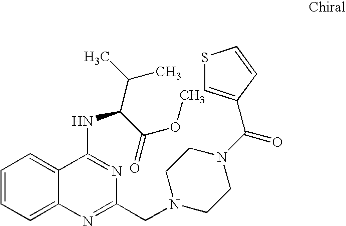 | 467.6 | 468.3 |
| 18-15 | 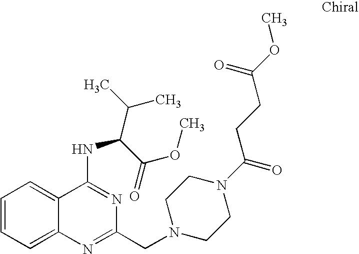 | 471.6 | 472.3 |
| 18-16 | 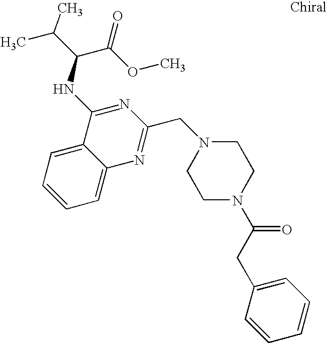 | 475.6 | 476.3 |
| 18-17 | 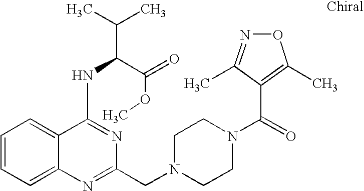 | 480.6 | 481.3 |
| 18-18 | 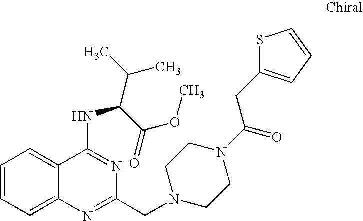 | 481.6 | 482.3 |
| 18-19 | 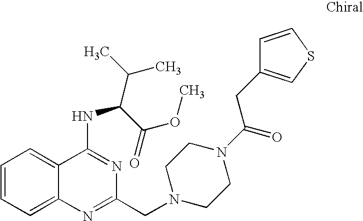 | 481.6 | 482.3 |
| 18-20 | 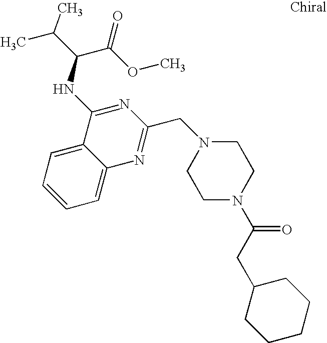 | 481.6 | 482.3 |
| 18-21 | 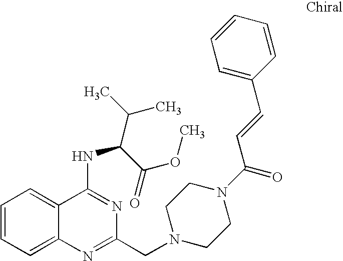 | 487.6 | 488.3 |
| 18-22 | 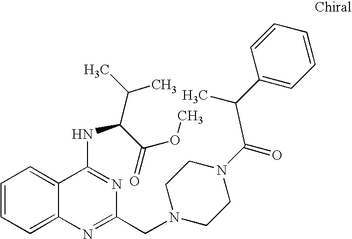 | 489.6 | 490.3 |
| 18-23 | 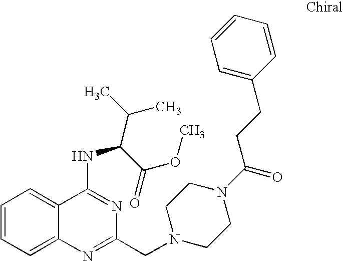 | 489.6 | 490.3 |
| 18-24 | 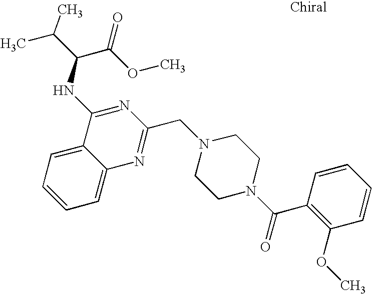 | 491.6 | 492.3 |
| 18-25 | 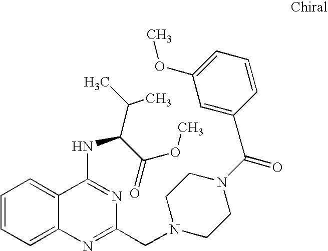 | 491.6 | 492.3 |
| 18-26 | 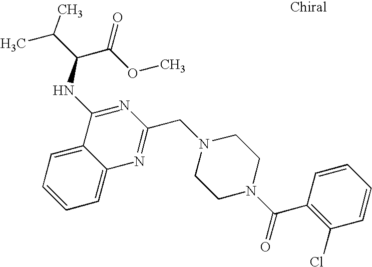 | 496.0 | 496.3 |
| 18-27 | 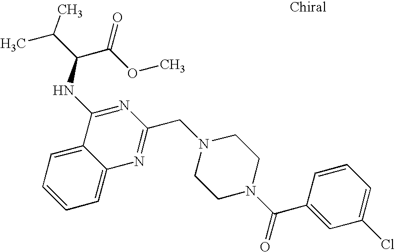 | 496.0 | 496.3 |
| 18-28 | 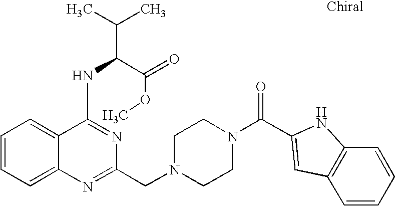 | 500.6 | 501.3 |
| 18-29 | 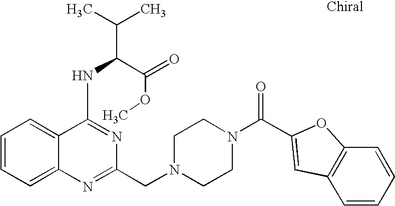 | 501.6 | 502.3 |
| 18-30 | 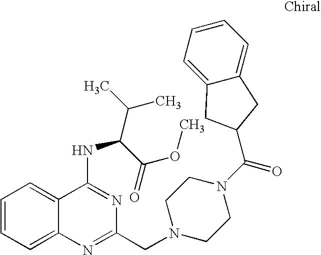 | 501.6 | 502.3 |
| 18-31 | 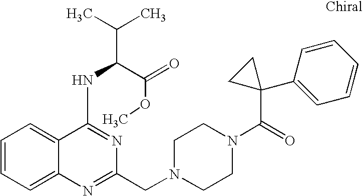 | 501.6 | 502.3 |
| 18-32 | 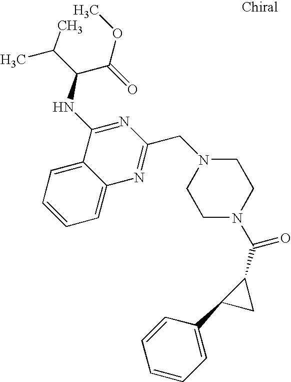 | 501.6 | 502.3 |
| 18-33 | 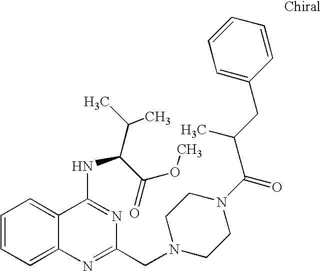 | 503.6 | 504.3 |
| 18-34 | 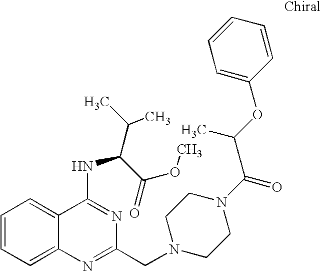 | 505.6 | 506.3 |
| 18-35 | 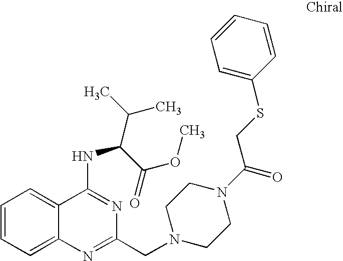 | 507.7 | 508.3 |
| 18-36 | 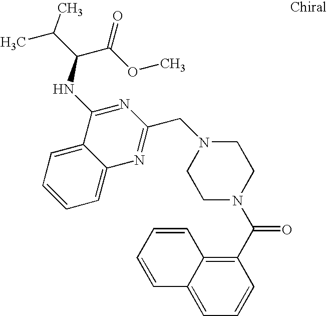 | 511.6 | 512.3 |
| 18-37 | 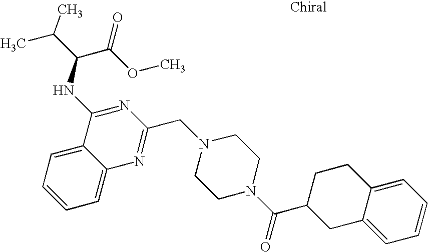 | 515.7 | 516.3 |
| 18-38 | 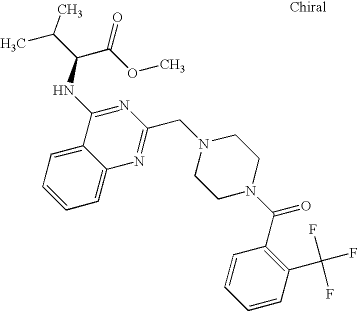 | 529.6 | 530.3 |
| 18-39 | 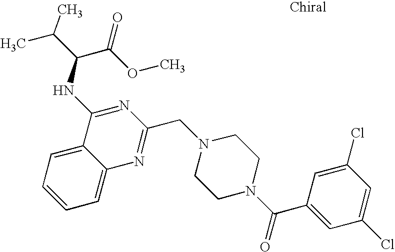 | 530.5 | 530.3 |
| 18-40 | 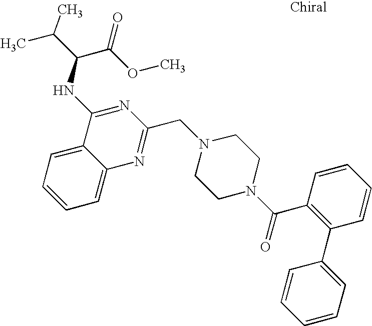 | 537.7 | 538.3 |
| 18-41 | 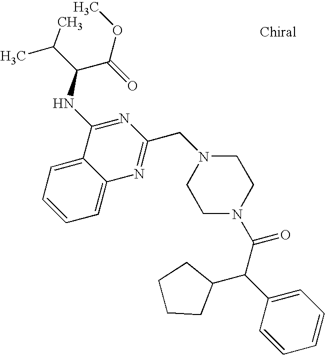 | 543.7 | 544.3 |
| 18-42 | 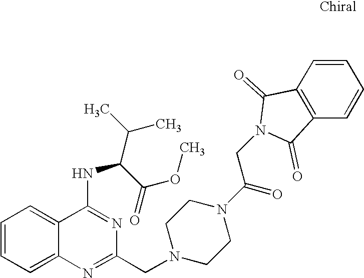 | 544.6 | 545.3 |
| 18-43 | 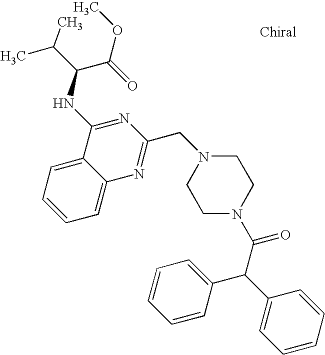 | 551.7 | 552.3 |
| 18-44 | 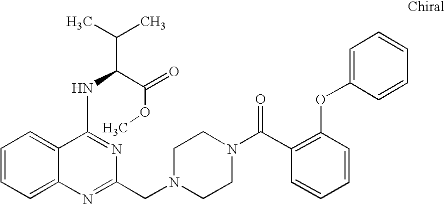 | 553.7 | 554.3 |
| 18-45 | 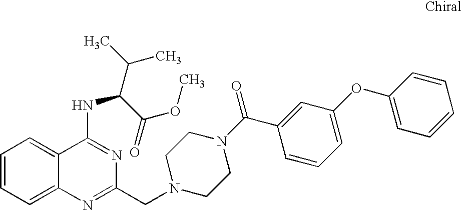 | 553.7 | 554.3 |
| 18-46 | 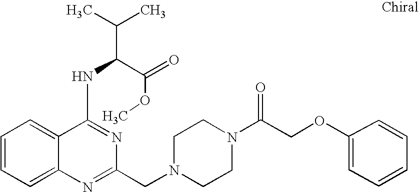 | 491.6 | 492.3 |
| 18-47 | 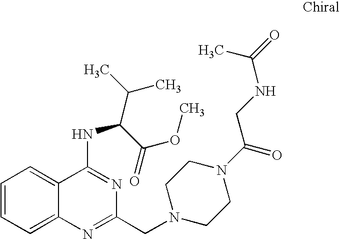 | 456.5 | 457.3 |
| 18-48 | 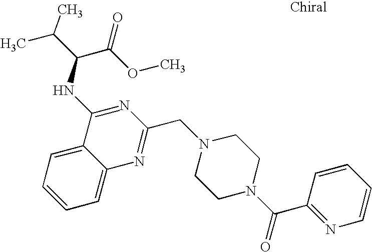 | 462.6 | 463.3 |
| 18-49 | 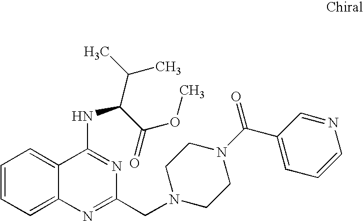 | 462.6 | 463.3 |
| 18-50 | 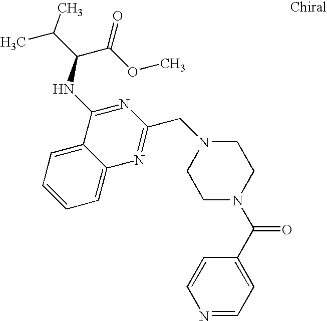 | 462.6 | 463.3 |
| 18-51 | 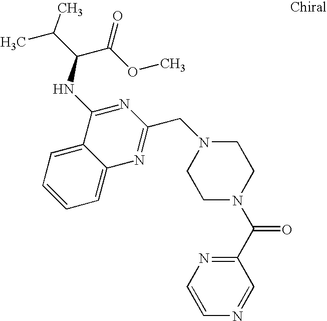 | 463.5 | 464.3 |
| 18-52 | 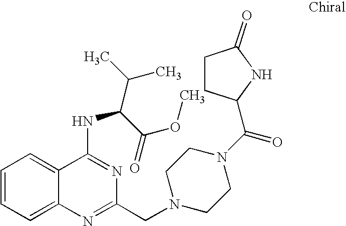 | 468.6 | 469.3 |
| 18-53 | 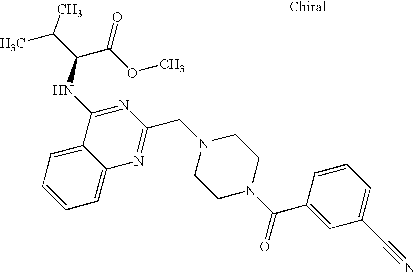 | 486.6 | 487.3 |
| 18-54 | 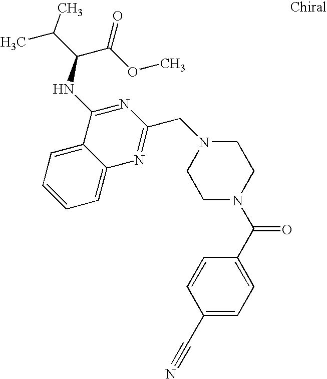 | 486.6 | 487.3 |
| 18-55 | 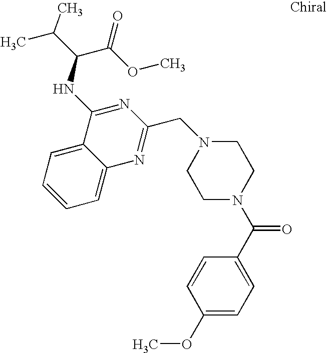 | 491.6 | 492.3 |
| 18-56 | 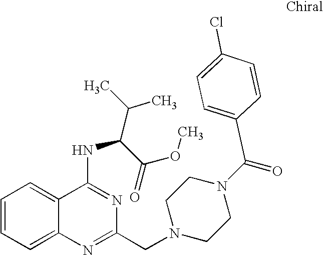 | 496.0 | 496.3 |
| 18-57 | 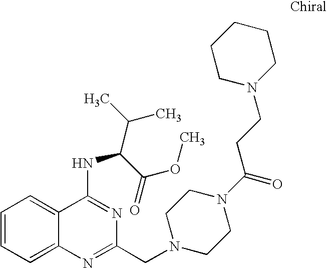 | 496.7 | 497.3 |
| 18-58 | 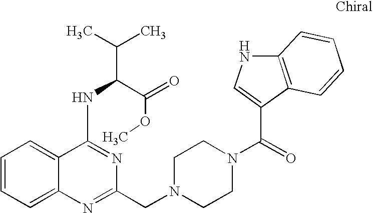 | 500.6 | 501.3 |
| 18-59 | 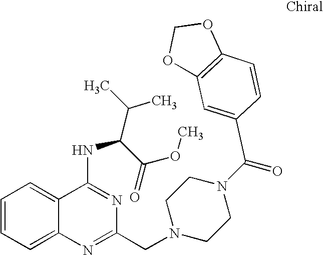 | 505.6 | 506.3 |
| 18-60 | 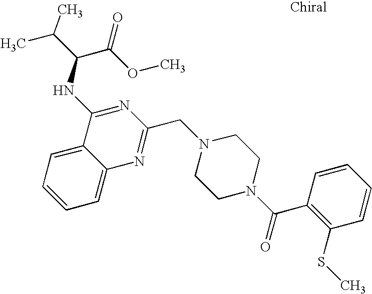 | 507.7 | 508.3 |
| 18-61 | 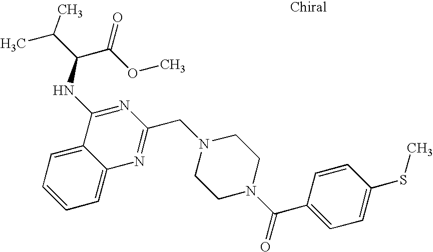 | 507.7 | 508.3 |
| 18-62 | 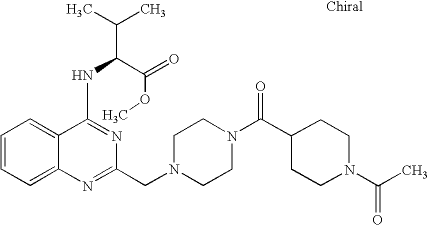 | 510.6 | 511.3 |
| 18-63 | 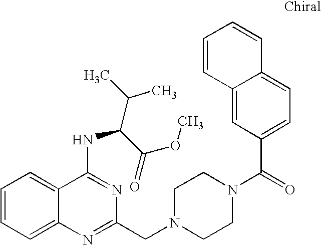 | 511.6 | 512.3 |
| 18-64 | 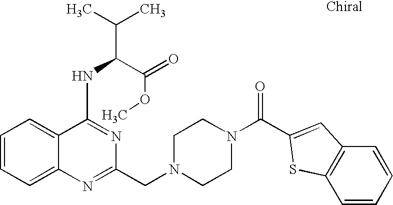 | 517.7 | 518.3 |
| 18-65 | 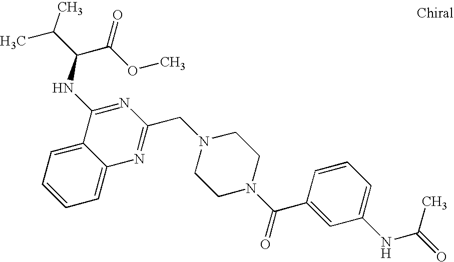 | 518.6 | 519.3 |
| 18-66 | 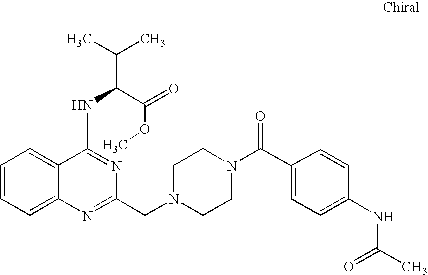 | 518.6 | 519.3 |
| 18-67 | 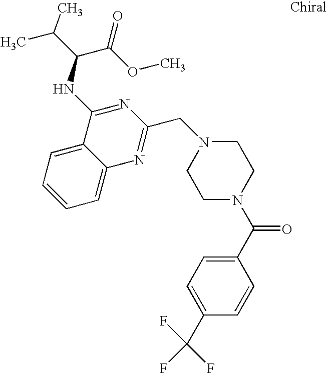 | 529.6 | 530.3 |
| 18-68 | 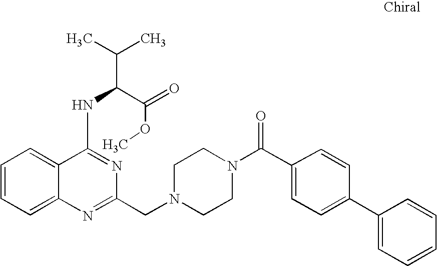 | 537.7 | 538.3 |
| 18-69 | 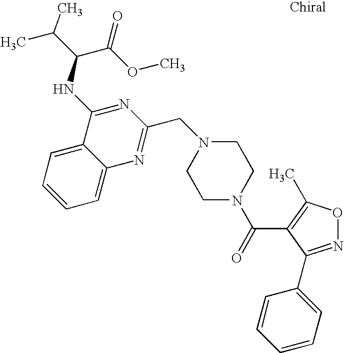 | 542.6 | 543.3 |
| 18-70 | 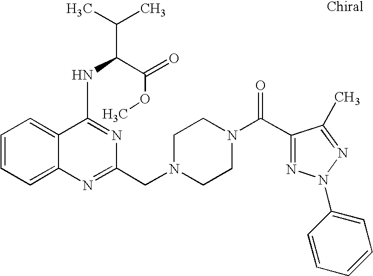 | 542.6 | 543.3 |
| 18-71 | 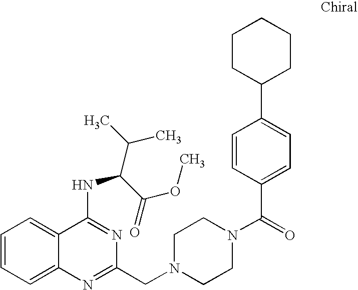 | 543.7 | 544.3 |
| 18-72 | 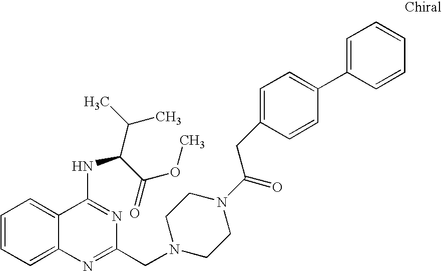 | 551.7 | 552.3 |

### Example 19

### Reaction of 3-METHYL-2(S)-(−)-[2-(PIPERAZIN-1-YLMETHYL)QUINAZOLIN-4-YLAMINO]BUTYRIC ACID METHYL ESTER with a SULFONYL CHLORIDE LIBRARY

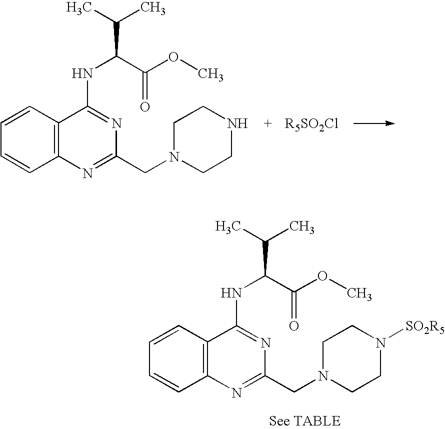

A stock solution of 3-methyl-2(S)-(−)-[2-(piperazin-1-ylmethyl)quinazolin-4-ylamino]butyric acid methyl ester (1 mL, 0.0233 mmoles) (prepared as described in Preparative Example 21 above) in THF was added to the tubes in three 24 tube Bohdan Miniblocks. PS-DIEA resin (3 equivalents, 0.0699 mmoles) was added to all of the tubes, followed by a 1M solution of each of the sulfonyl chlorides (R₅SO₂Cl) (1.5 equivalents, 0.0350 mmoles) in THF. Additional THF (0.5 mL) was added to each tube and the Miniblocks were sealed and shaken at 25° C. for 22 h. PS-Isocyanate resin (3 equivalents, 0.0699 mmoles) was added to each tube, followed by PS-Trisamine resin (6 equivalents, 0.14 mmoles) and the Miniblocks were shaken at 25° C. for 4 h. The tubes were drained and the resin was washed with THF (2×1 mL). The combined filtrates from each tube, were evaporated to dryness overnight, on a Speedvac concentrator. The resulting samples were evaluated by LCMS and those that were >70% pure are listed in the table below.

### Examples 19-1 Through 19-76

| EXAMPLE | STRUCTURE | MW | LCMS m/z |
|---|---|---|---|
| 19-1 | 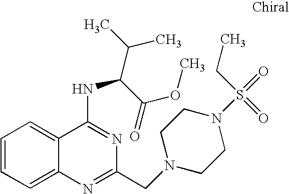 | 449.6 | 450.2 |
| 19-2 | 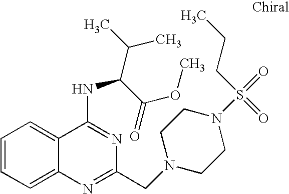 | 463.6 | 464.3 |
| 19-3 | 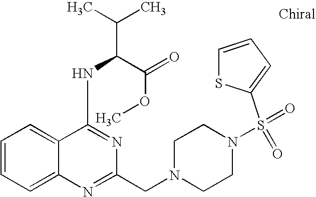 | 503.6 | 504.3 |
| 19-4 | 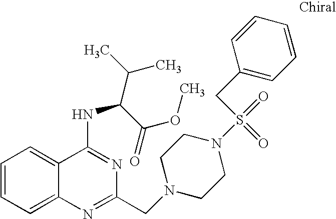 | 511.6 | 512.3 |
| 19-5 | 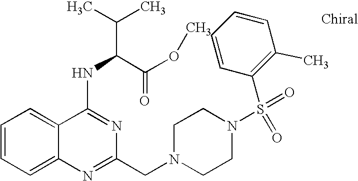 | 511.6 | 512.3 |
| 19-6 | 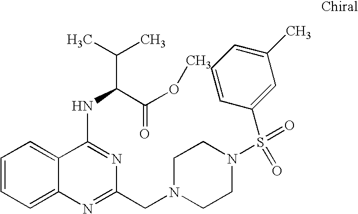 | 511.6 | 512.3 |
| 19-7 | 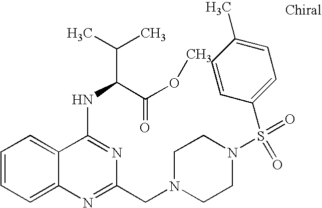 | 511.6 | 512.3 |
| 19-8 | 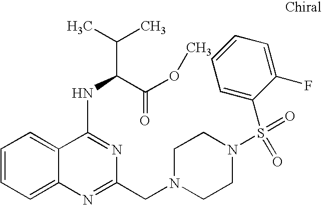 | 515.6 | 516.3 |
| 19-9 | 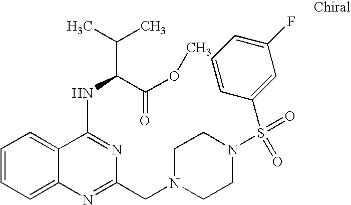 | 515.6 | 516.3 |
| 19-10 | 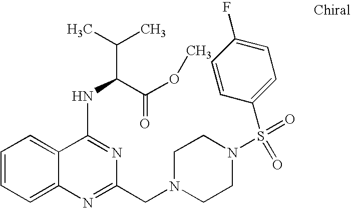 | 515.6 | 516.3 |
| 19-11 | 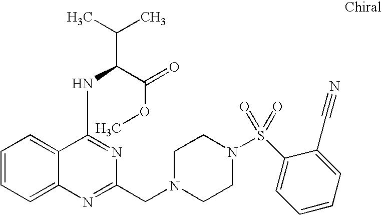 | 522.6 | 523.3 |
| 19-12 | 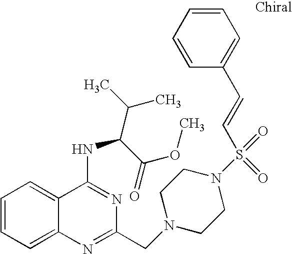 | 523.7 | 524.3 |
| 19-13 | 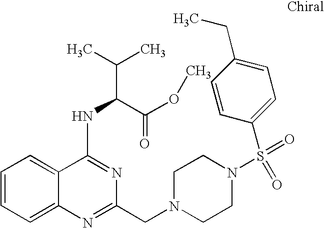 | 525.7 | 526.3 |
| 19-14 | 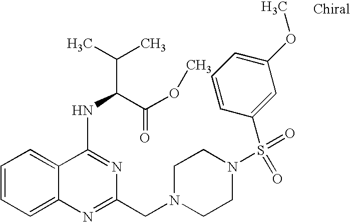 | 527.6 | 528.3 |
| 19-15 | 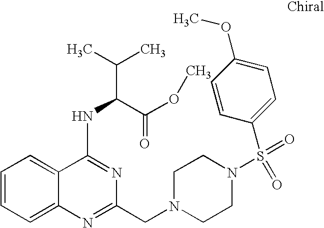 | 527.6 | 528.3 |
| 19-16 | 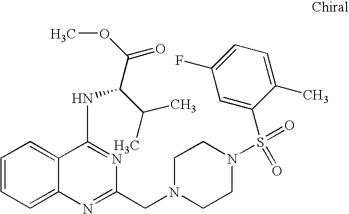 | 529.6 | 530.3 |
| 19-17 | 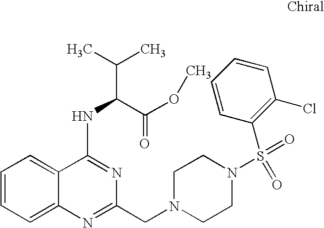 | 532.1 | 532.3 |
| 19-18 | 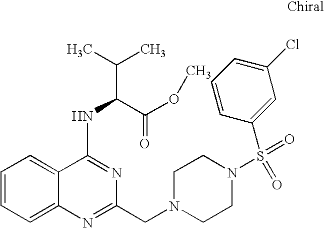 | 532.1 | 532.3 |
| 19-19 | 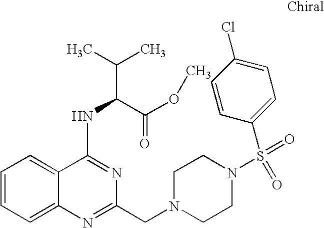 | 532.1 | 532.3 |
| 19-20 | 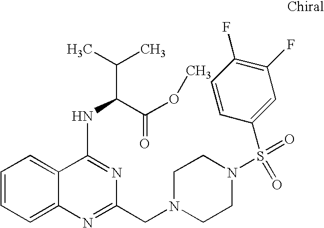 | 533.6 | 534.3 |
| 19-21 | 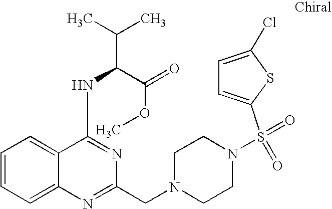 | 538.1 | 538.3 |
| 19-22 | 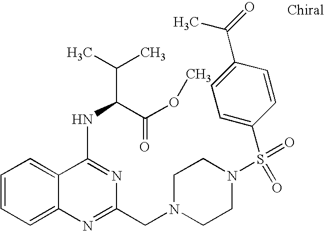 | 539.7 | 540.3 |
| 19-23 | 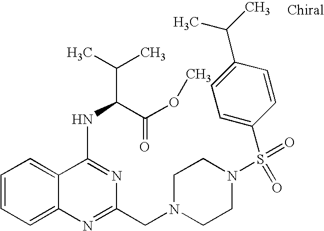 | 539.7 | 540.3 |
| 19-24 | 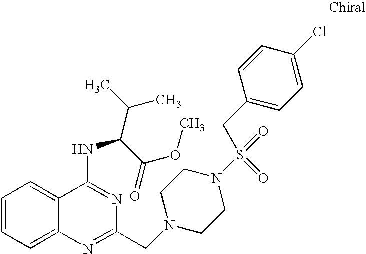 | 546.1 | 546.3 |
| 19-25 | 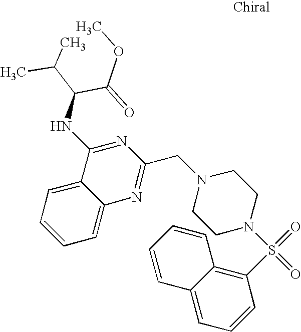 | 547.7 | 548.3 |
| 19-26 | 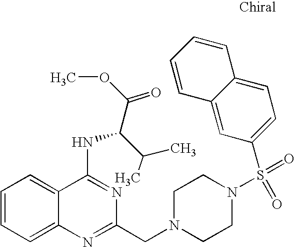 | 547.7 | 548.3 |
| 19-27 | 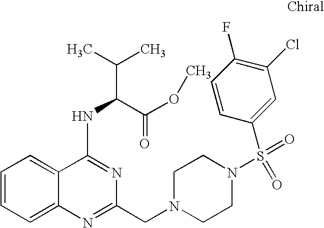 | 550.1 | 550.3 |
| 19-28 | 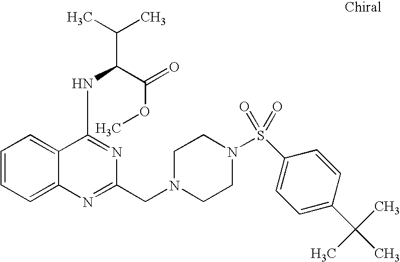 | 553.7 | 554.3 |
| 19-29 | 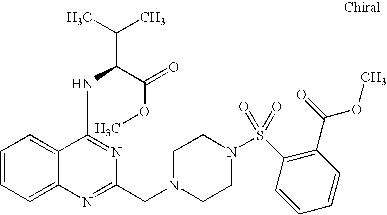 | 555.7 | 556.3 |
| 19-30 | 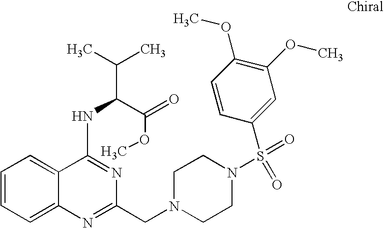 | 557.7 | 558.3 |
| 19-31 | 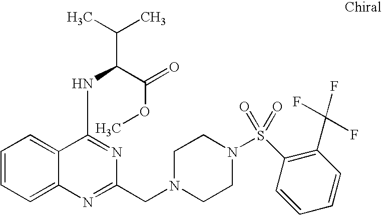 | 565.6 | 566.3 |
| 19-32 | 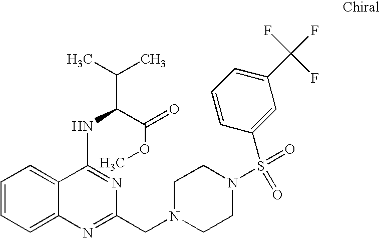 | 565.6 | 566.3 |
| 19-33 | 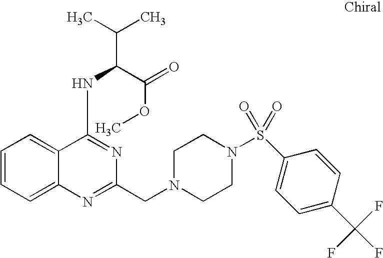 | 565.6 | 566.3 |
| 19-34 | 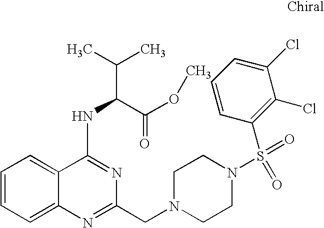 | 566.5 | 566.3 |
| 19-35 | 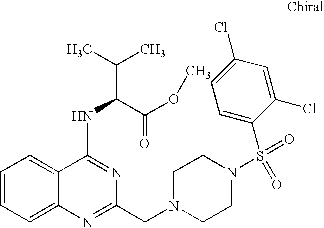 | 566.5 | 566.3 |
| 19-36 | 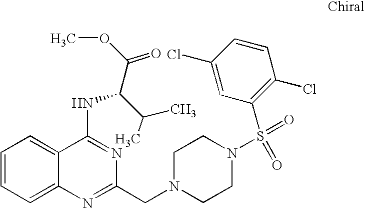 | 566.5 | 566.3 |
| 19-37 | 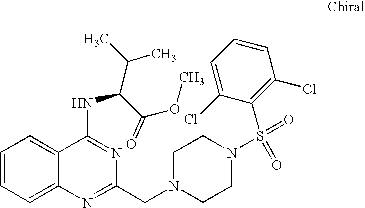 | 566.5 | 566.3 |
| 19-38 | 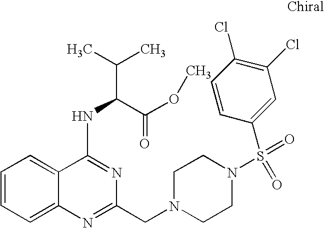 | 566.5 | 566.3 |
| 19-39 | 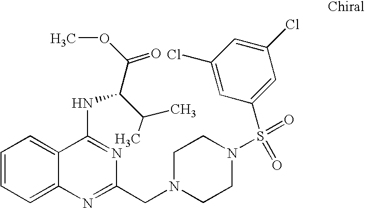 | 566.5 | 566.3 |
| 19-40 | 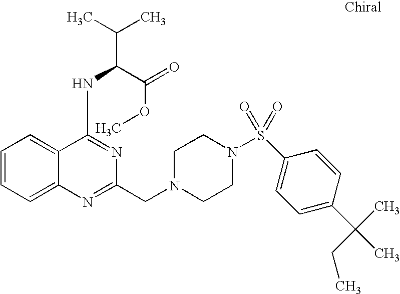 | 567.8 | 568.3 |
| 19-41 | 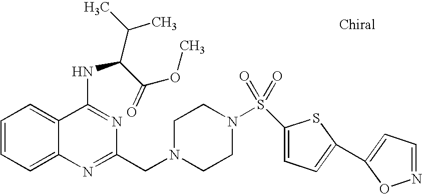 | 570.7 | 571.3 |
| 19-42 | 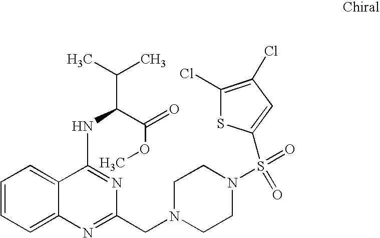 | 572.5 | 572.3 |
| 19-43 | 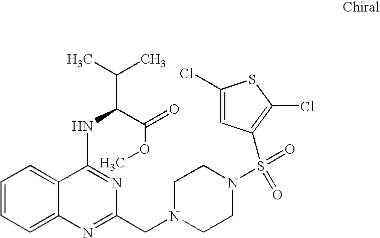 | 572.5 | 572.3 |
| 19-44 | 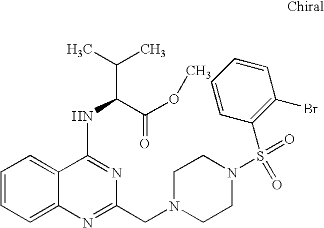 | 576.5 | 578.3 |
| 19-45 | 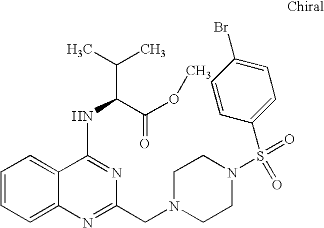 | 576.5 | 578.3 |
| 19-46 | 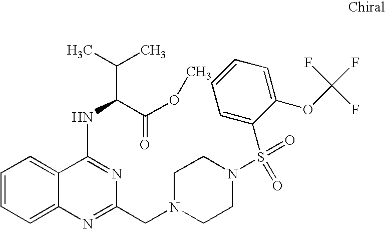 | 581.6 | 582.3 |
| 19-47 | 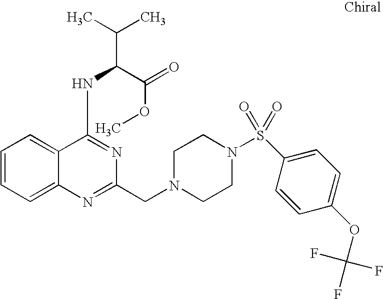 | 581.6 | 582.3 |
| 19-48 | 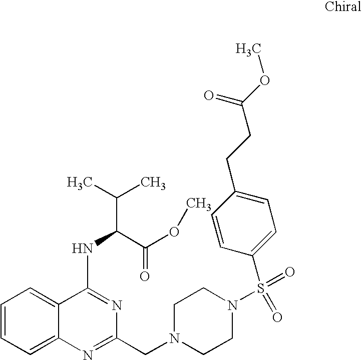 | 583.7 | 584.3 |
| 19-49 | 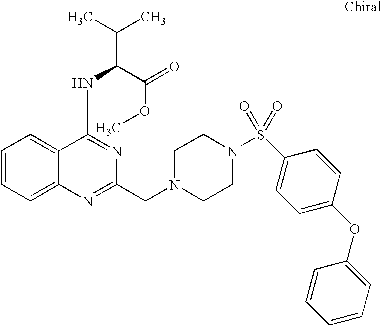 | 589.7 | 590.3 |
| 19-50 | 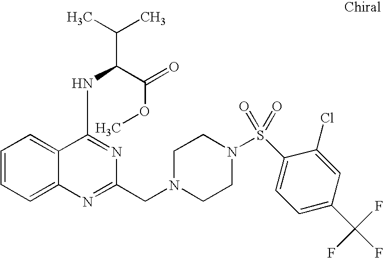 | 600.1 | 600.3 |
| 19-51 | 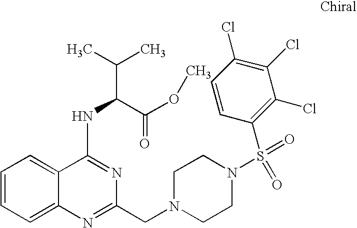 | 601.0 | 602.3 |
| 19-52 | 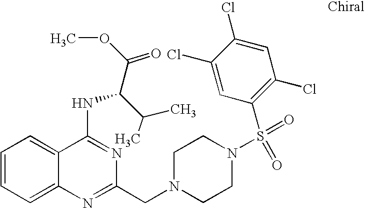 | 601.0 | 602.3 |
| 19-53 | 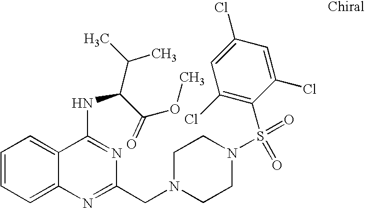 | 601.0 | 602.3 |
| 19-54 | 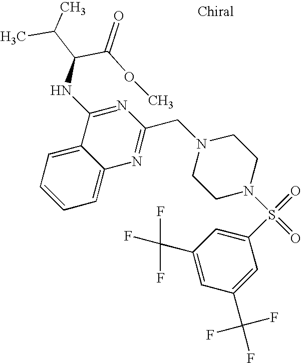 | 633.6 | 634.3 |
| 19-55 | 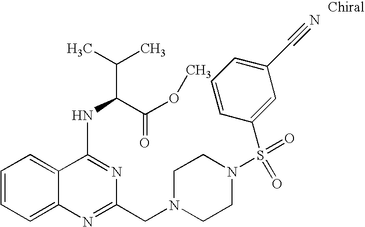 | 522.6 | 523.3 |
| 19-56 | 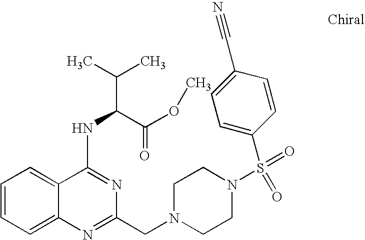 | 522.6 | 523.3 |
| 19-57 | 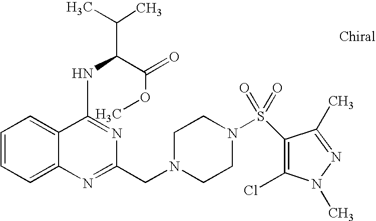 | 550.1 | 550.3 |
| 19-58 | 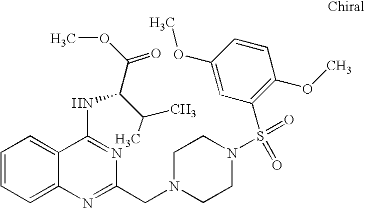 | 557.7 | 558.3 |
| 19-59 | 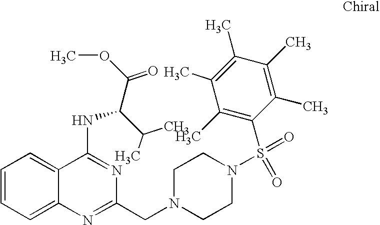 | 567.8 | 568.3 |
| 19-60 | 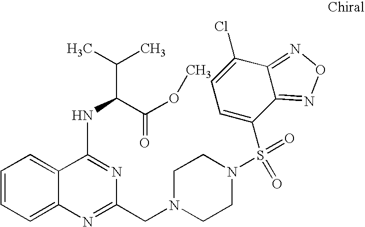 | 574.1 | 574.3 |
| 19-61 | 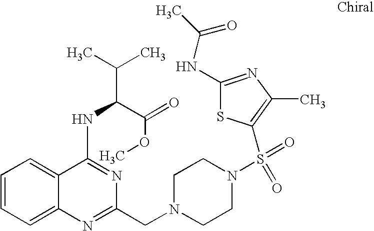 | 575.7 | 576.3 |
| 19-62 | 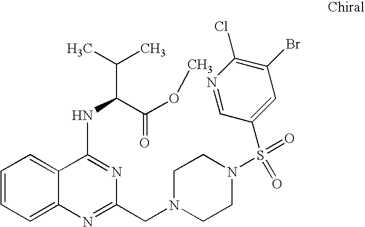 | 612.0 | 613.3 |
| 19-63 | 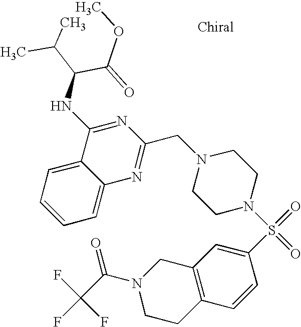 | 648.7 | 649.4 |
| 19-64 | 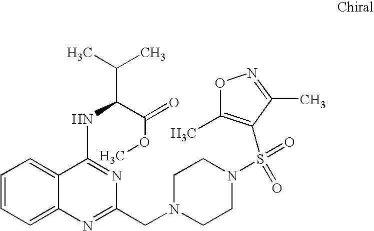 | 516.6 | 517.3 |
| 19-65 | 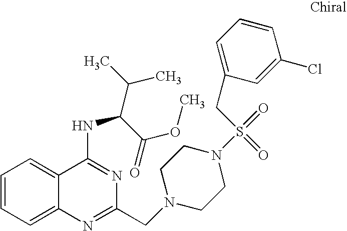 | 546.1 | 546.3 |
| 19-66 | 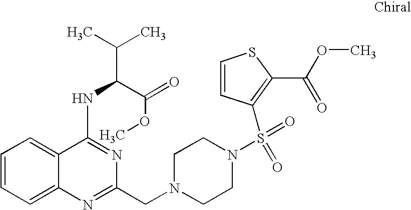 | 561.7 | 562.3 |
| 19-67 | 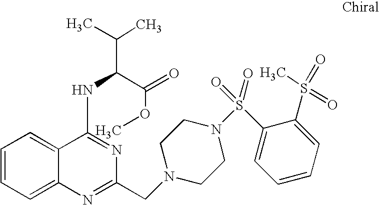 | 575.7 | 576.3 |
| 19-68 | 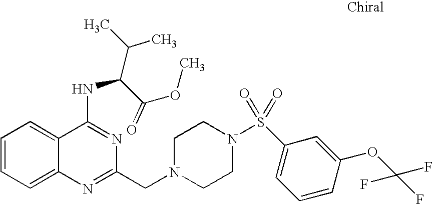 | 581.6 | 582.3 |
| 19-69 | 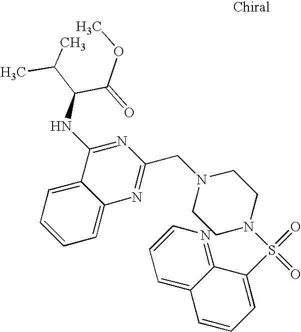 | 548.7 | 549.3 |
| 19-70 | 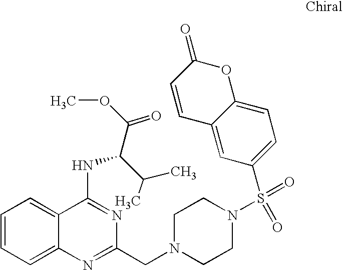 | 565.7 | 566.3 |
| 19-71 | 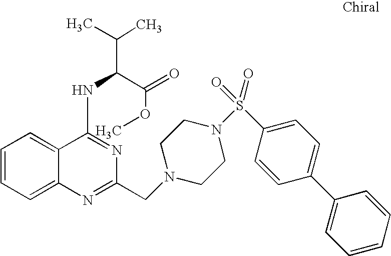 | 573.7 | 574.3 |
| 19-72 | 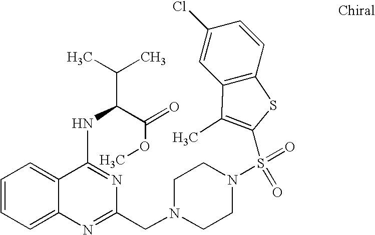 | 602.2 | 602.3 |
| 19-73 | 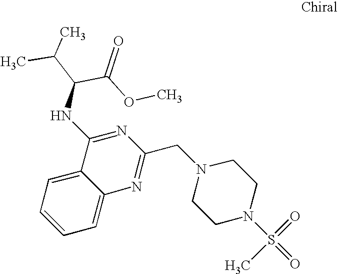 | 435.5 | 436.2 |
| 19-74 | 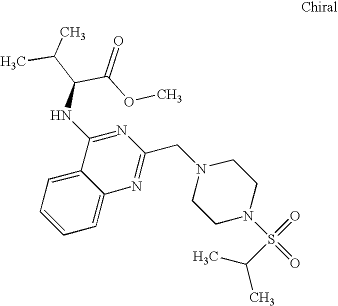 | 463.6 | 464.3 |
| 19-75 | 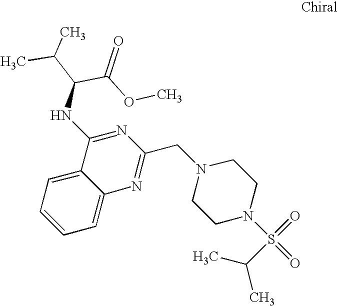 | 497.6 | 498.3 |
| 19-76 | 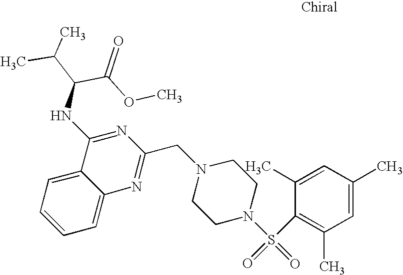 | 539.7 | 540.3 |

### Example 20

### Reaction of 3-METHYL-2(S)-(−)-[2-(PIPERAZIN-1-YLMETHYL)QUINAZOLIN-4-YLAMINO]BUTYRIC ACID METHYL ESTER with an ISOCYANATE LIBRARY

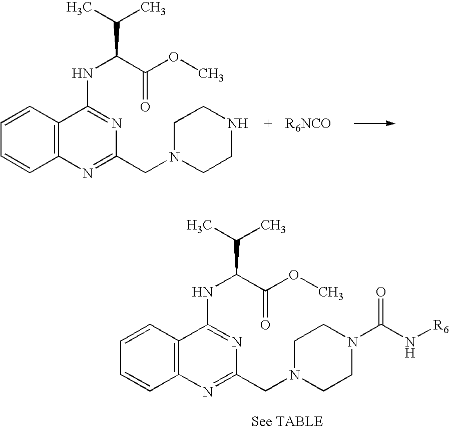

A stock solution of 3-methyl-2(S)-(−)-[2-(piperazin-1-ylmethyl)quinazolin-4-ylamino]butyric acid methyl ester (1 mL, 0.0233 mmoles) (prepared as described in Preparative Example 21 above) in THF was added to the tubes in three 24 tube Bohdan Miniblocks. A 1M stock solution of each of the isocyanates (R₆NCO) in THF (0.05 mL, 0.0466 mmoles) was added to each tube. PS-Isocyanate resin (3 equivalents, 0.0699 mmoles) was added to each tube, followed by PS-Trisamine resin (6 equivalents, 0.14 mmoles) and the Miniblocks were shaken at 25° C. for 18 h. The blocks were drained onto MP-TsOH resin (4 equivalents, 0.0932 mmoles) and the PS-Trisamine resins were washed with THF. The Miniblocks were shaken at 25° C. for 4 h and then drained and the resin was washed with dichloromethane and the filtrates were discarded. Ammonia in methanol (2N, 2 mL) was added to each tube and the Miniblocks were shaken at 25° C. for 4 h. The methanol filtrates were collected and the resin was again shaken with ammonia in methanol (2N, 2 mL). The combined filtrates from each tube were evaporated to dryness overnight, on a Speedvac concentrator. The resulting samples were analyzed by LCMS and those that were >70% pure are listed in the table below.

### Examples 20-1 Through 20-50

| | | | | | SPA |
|---|---|---|---|---|---|
| | | | | PROLIFE- | ASSAY |
| | | | | RATION | (% |
| | | | | ASSAY | residual |
| | | | | EC50 | T @ |
| COMPOUND | | | LCMS | MB468 | 2 ug/mL |
| # | STRUCTURE | MW | m/z | (uM) | of drug) |
| 20-1 | 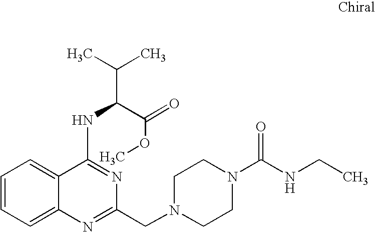 | 428.5 | 429.2 | | |
| 20-2 | 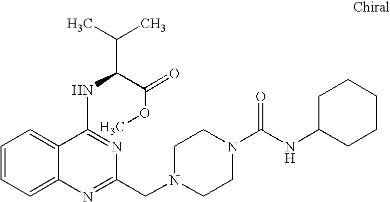 | 482.6 | 483.3 | | |
| 20-3 | 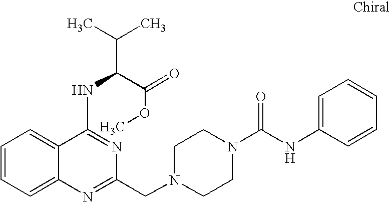 | 476.6 | 477.3 | | |
| 20-4 | 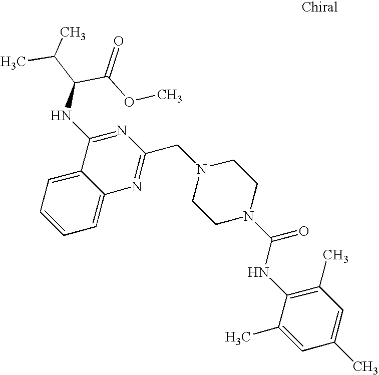 | 518.7 | 519.3 | | |
| 20-5 | 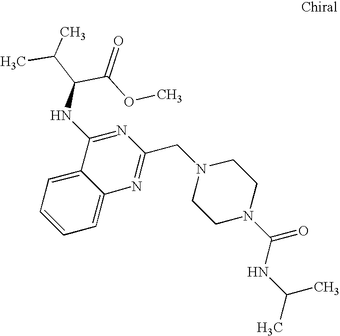 | 442.6 | 443.2 | | |
| 20-6 | 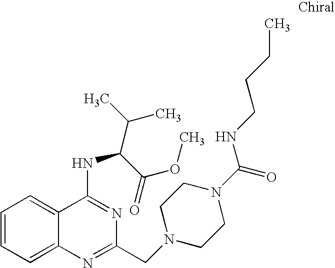 | 456.6 | 457.3 | | |
| 20-7 | 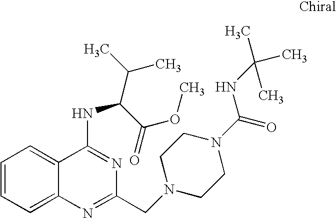 | 456.6 | 457.3 | | |
| 20-8 | 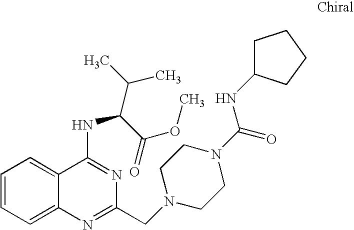 | 468.6 | 469.3 | | |
| 20-9 | 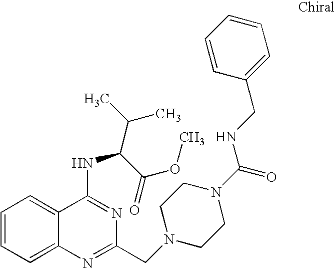 | 490.6 | 491.3 | | |
| 20-10 | 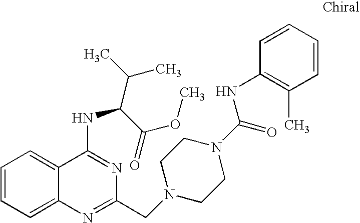 | 490.6 | 491.3 | | |
| 20-11 | 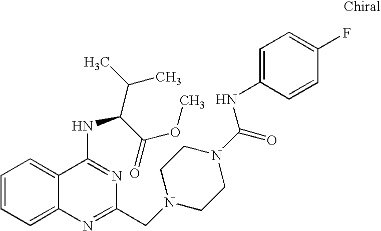 | 494.6 | 495.3 | | |
| 20-12 | 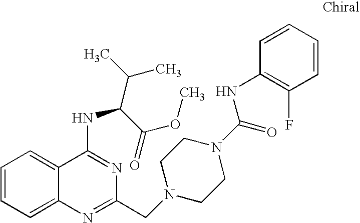 | 494.6 | 495.3 | | |
| 20-13 | 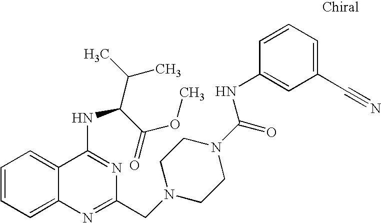 | 501.6 | 502.3 | | |
| 20-14 | 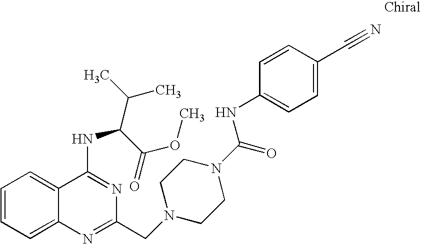 | 501.6 | 502.3 | | |
| 20-15 | 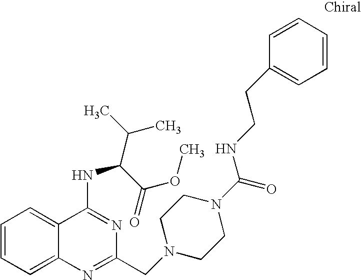 | 504.6 | 505.3 | | |
| 20-16 | 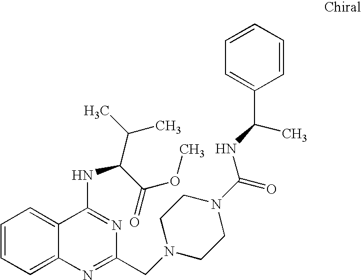 | 504.6 | 505.3 | | |
| 20-17 | 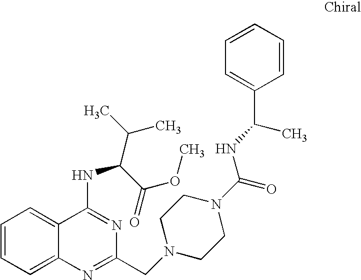 | 504.6 | 505.3 | | |
| 20-18 | 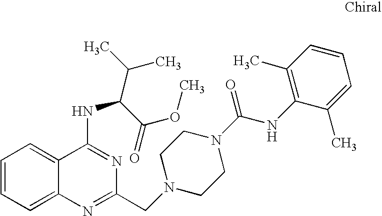 | 504.6 | 505.3 | | |
| 20-19 | 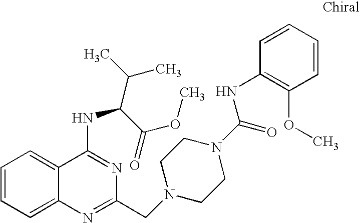 | 506.6 | 507.3 | | |
| 20-20 | 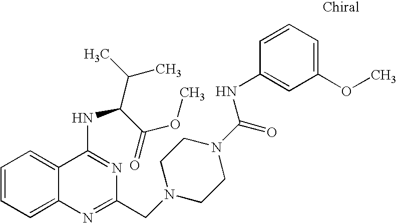 | 506.6 | 507.3 | | |
| 20-21 | 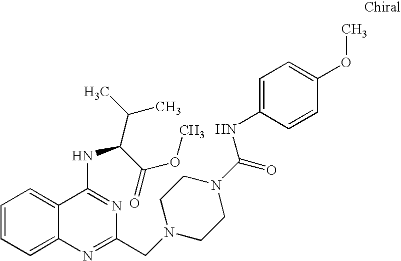 | 506.6 | 507.3 | | |
| 20-22 | 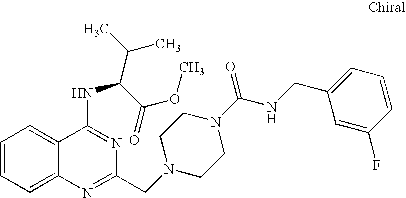 | 508.6 | 509.3 | | |
| 20-23 | 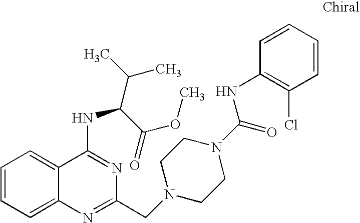 | 511.0 | 511.3 | | |
| 20-24 | 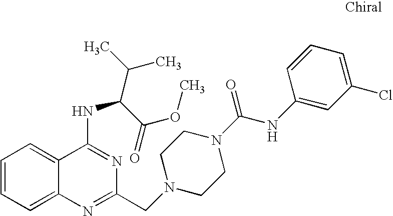 | 511.0 | 511.3 | | |
| 20-25 | 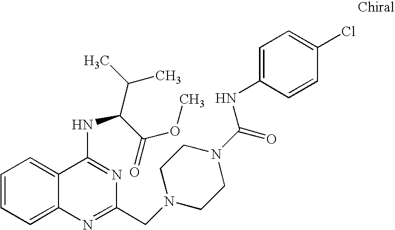 | 511.0 | 511.3 | | |
| 20-26 | 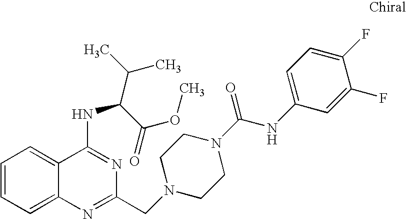 | 512.6 | 513.3 | | |
| 20-27 | 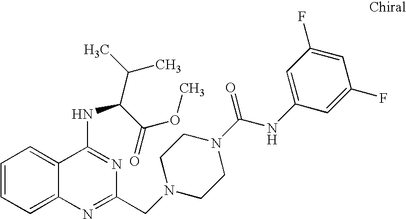 | 512.6 | 513.3 | | |
| 20-28 | 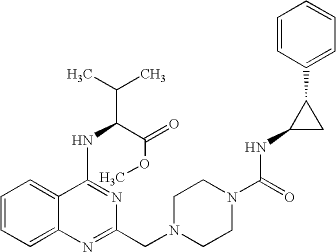 | 516.6 | 517.3 | | |
| 20-29 | 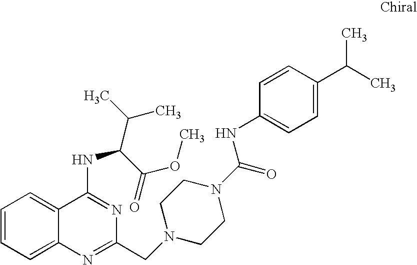 | 518.7 | 519.3 | C | B |
| 20-30 | 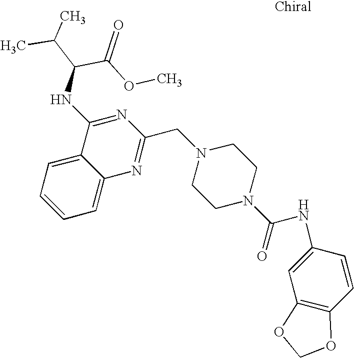 | 520.6 | 521.3 | | |
| 20-31 | 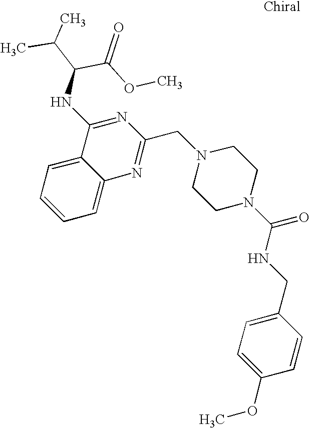 | 520.6 | 521.3 | | |
| 20-32 | 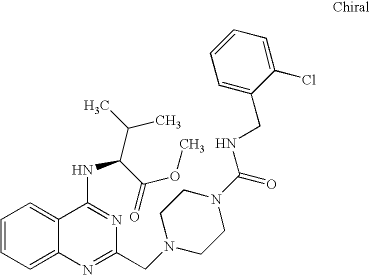 | 525.1 | 525.3 | | |
| 20-33 | 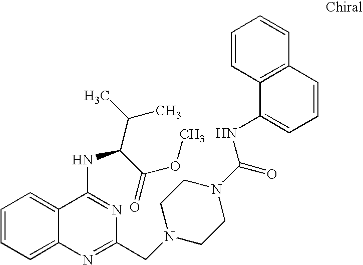 | 526.6 | 527.3 | | |
| 20-34 | 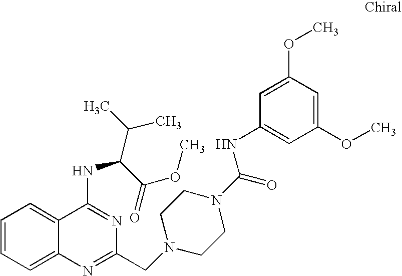 | 536.6 | 537.3 | | |
| 20-35 | 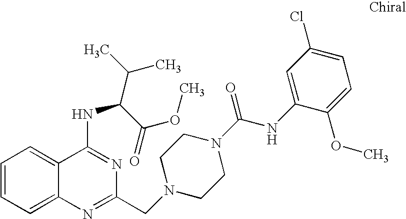 | 541.1 | 541.3 | | |
| 20-36 | 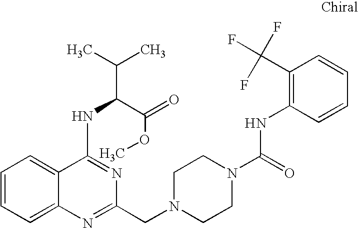 | 544.6 | 545.3 | | |
| 20-37 | 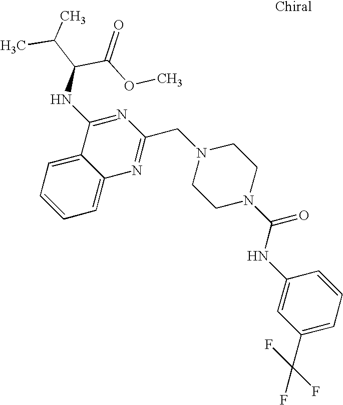 | 544.6 | 545.3 | | |
| 20-38 | 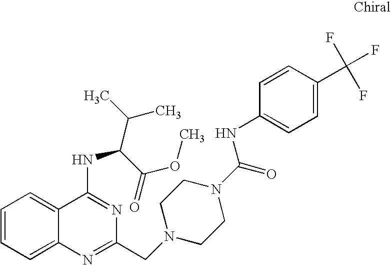 | 544.6 | 545.3 | | |
| 20-39 | 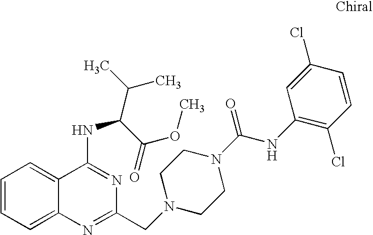 | 545.5 | 545.3 | | |
| 20-40 | 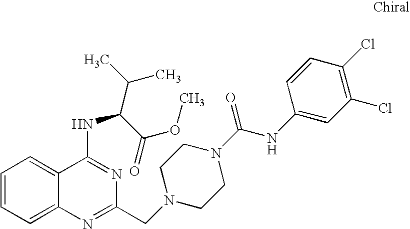 | 545.5 | 545.3 | | |
| 20-41 | 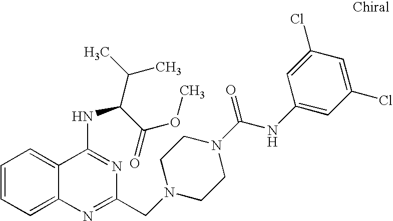 | 545.5 | 545.3 | | |
| 20-42 | 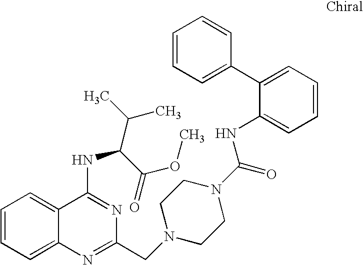 | 552.7 | 553.3 | | |
| 20-43 | 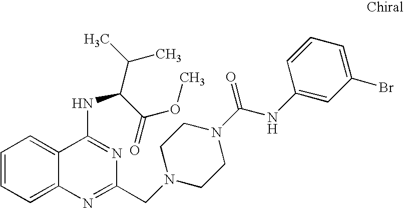 | 555.5 | 555.3 | | |
| 20-44 | 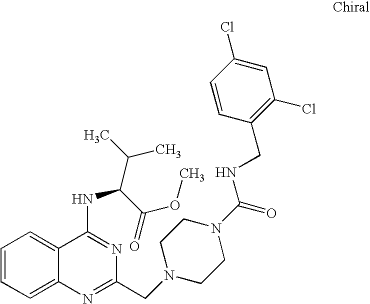 | 559.5 | 559.3 | | |
| 20-45 | 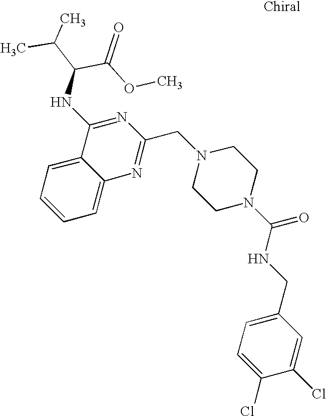 | 559.5 | 559.3 | | |
| 20-46 | 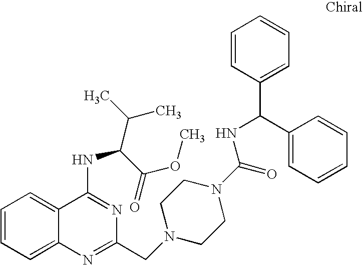 | 566.7 | 567.3 | | |
| 20-47 | 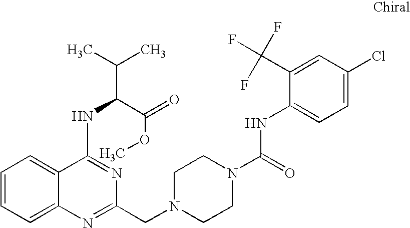 | 579.0 | 579.3 | | |
| 20-48 | 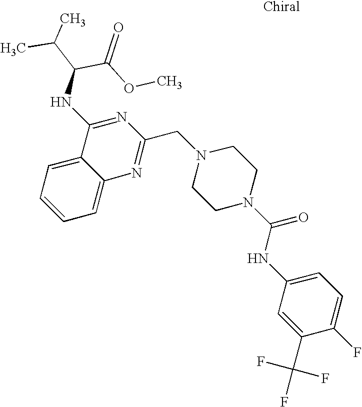 | 562.6 | 563.3 | | |
| 20-49 | 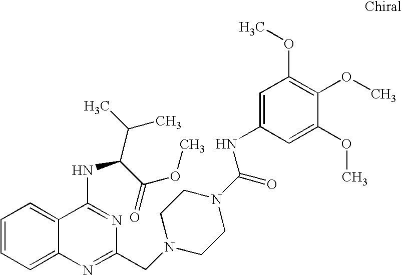 | 566.7 | 567.3 | | |
| 20-50 | 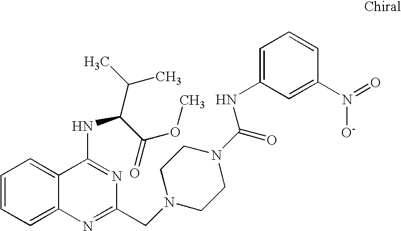 | 521.6 | 522.3 | | |

### Example 21

### Reaction of 2(S)-(−)-[2-(PIPERAZIN-1-YLMETHYL)QUINAZOLIN-4-YLAMINO]-3-METHYLBUTYRAMIDE with an ALDEHYDE/KETONE LIBRARY

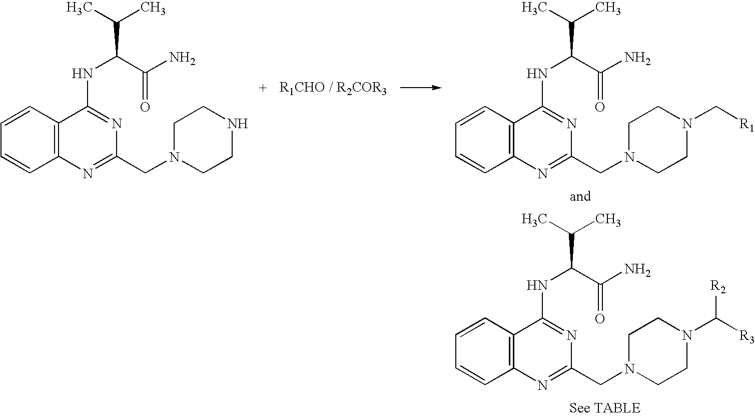

A stock solution of 2(S)-(−)-[2-(piperazin-1-ylmethyl)quinazolin-4-ylamino]-3-methylbutyramide (1 mL, 0.0277 mmoles) (prepared as described in Preparative Example 22 above) in THF was added to the tubes in five 24 tube Bohdan Miniblocks. A 1M stock solution of each of the individual aldehydes R₁CHO and ketones (R₂COR₃) in THF (0.1 mL, 0.0997 mmoles) was then added, one to each tube. Sodium triacetoxyborohydride (18 mg, 0.0859 mmoles) was added to each tube, followed by additional THF (0.5 mL). The Miniblocks were sealed and shaken at 25° C. for 20 h. Methanol (0.5 mL) and MP-TsOH resin (˜0.12 g) were added to each tube and the Miniblocks were shaken at 25° C. for 4 h. The tubes were drained and the resin was washed three times with methanol, shaking for 5 min each time, to remove unreacted reagents. Ammonia in methanol (2N, 2 mL) was added to each tube and the Miniblocks were again shaken at 25° C. for 20 min. The methanol filtrates were collected and the resin was again shaken with ammonia in methanol (2N, 2 mL) as described above. The combined filtrates from each tube, were evaporated to dryness overnight, on a Speedvac concentrator. The resulting samples were analyzed by LCMS and any samples that were <70% pure, were further purified by preparative LCMS. The compounds that were prepared that were >70% pure are listed in the table below.

### Examples 21-1 Through 21-97

| EXAMPLE | STRUCTURE | MW | LCMS m/z |
|---|---|---|---|
| 21-1 | 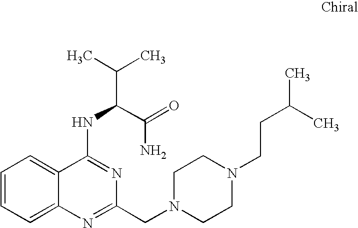 | 412.6 | 413.2 |
| 21-2 | 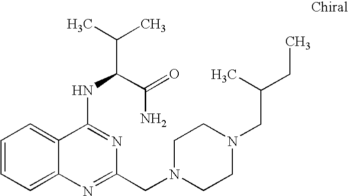 | 412.6 | 413.2 |
| 21-3 | 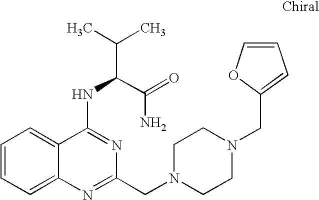 | 422.5 | 423.2 |
| 21-4 | 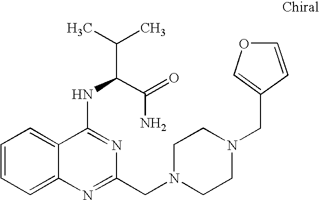 | 422.5 | 423.2 |
| 21-5 | 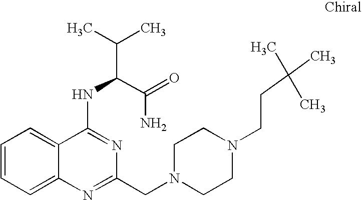 | 426.6 | 427.2 |
| 21-6 | 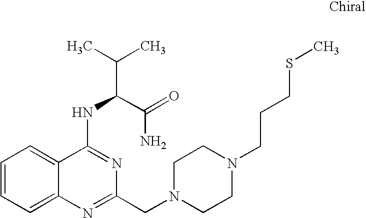 | 430.6 | 431.2 |
| 21-7 | 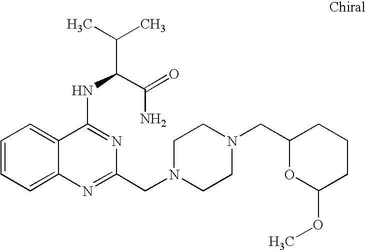 | 470.6 | 471.3 |
| 21-8 | 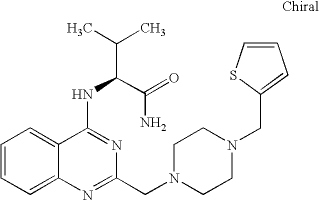 | 438.6 | 439.2 |
| 21-9 | 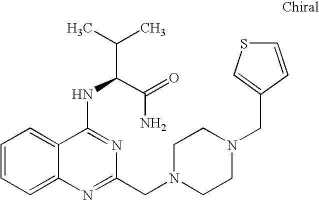 | 438.6 | 439.2 |
| 21-10 | 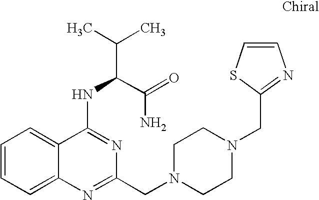 | 439.6 | 440.2 |
| 21-11 | 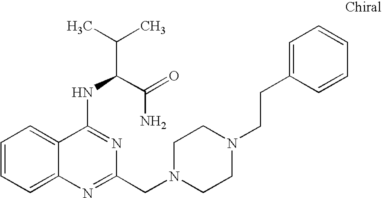 | 446.6 | 447.2 |
| 21-12 | 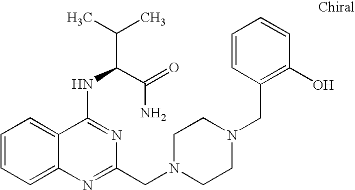 | 448.6 | 449.2 |
| 21-13 | 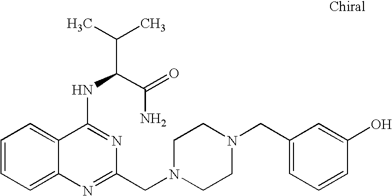 | 448.6 | 449.2 |
| 21-14 | 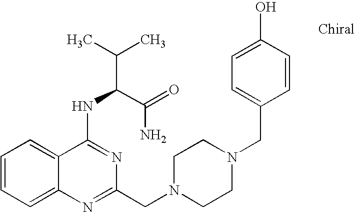 | 448.6 | 449.2 |
| 21-15 | 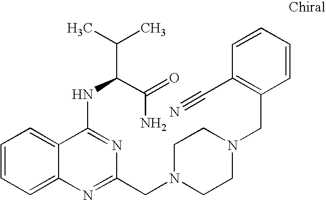 | 457.6 | 458.3 |
| 21-16 | 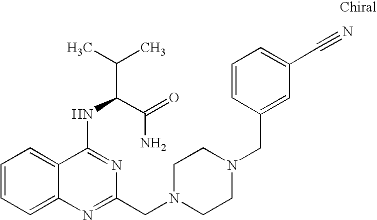 | 457.6 | 458.3 |
| 21-17 | 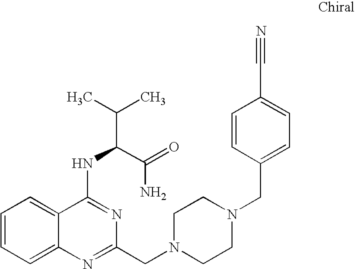 | 457.6 | 458.3 |
| 21-18 | 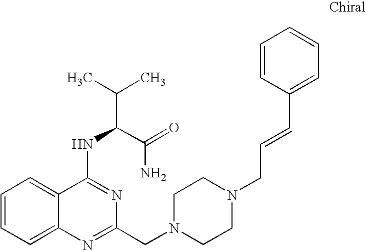 | 458.6 | 459.3 |
| 21-19 | 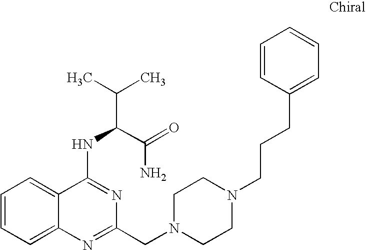 | 460.6 | 461.3 |
| 21-20 | 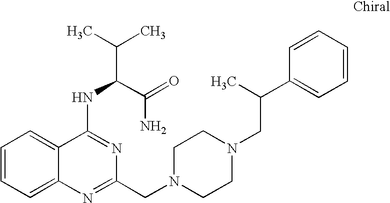 | 460.6 | 461.3 |
| 21-21 | 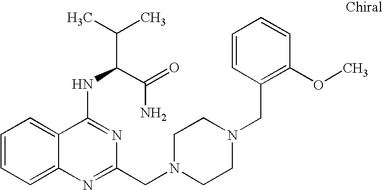 | 462.6 | 463.3 |
| 21-22 | 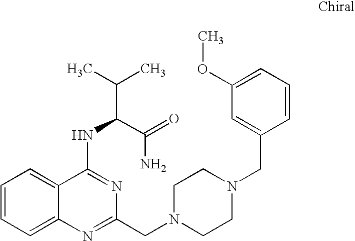 | 462.6 | 463.3 |
| 21-23 | 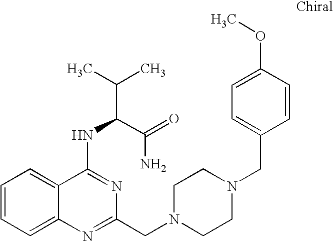 | 462.6 | 463.3 |
| 21-24 | 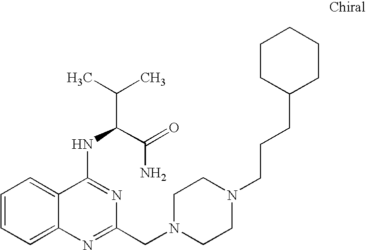 | 466.7 | 467.3 |
| 21-25 | 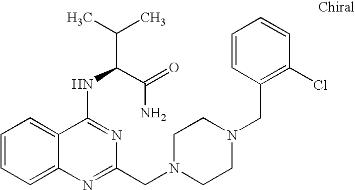 | 467.0 | 467.3 |
| 21-26 | 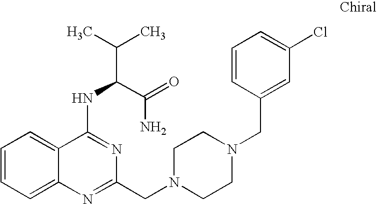 | 467.0 | 467.3 |
| 21-27 | 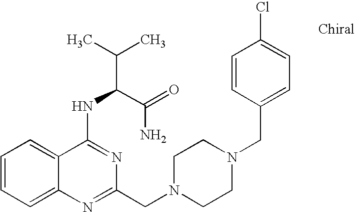 | 467.0 | 467.3 |
| 21-28 | 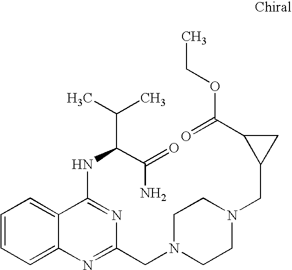 | 468.6 | 469.3 |
| 21-29 | 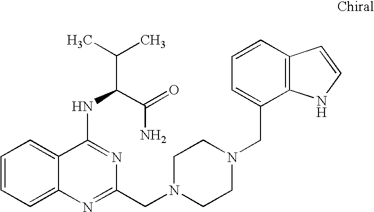 | 471.6 | 472.3 |
| 21-30 | 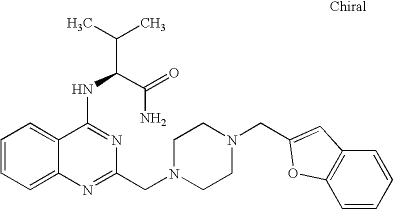 | 472.6 | 473.3 |
| 21-31 | 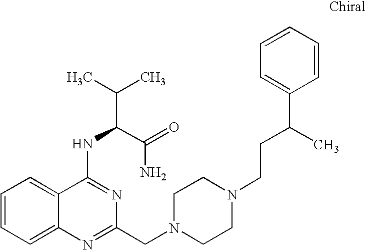 | 474.7 | 475.3 |
| 21-32 | 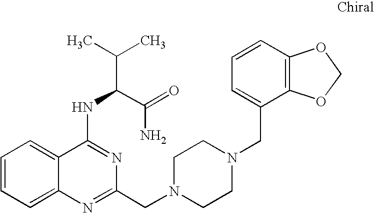 | 476.6 | 477.3 |
| 21-33 | 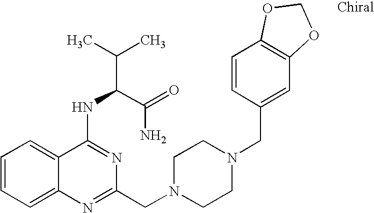 | 476.6 | 477.3 |
| 21-34 | 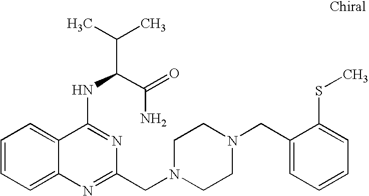 | 478.7 | 479.3 |
| 21-35 | 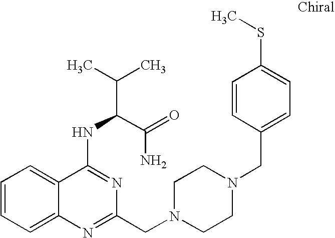 | 478.7 | 479.3 |
| 21-36 | 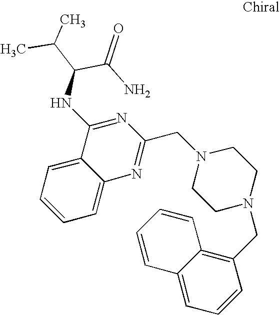 | 482.6 | 483.3 |
| 21-37 | 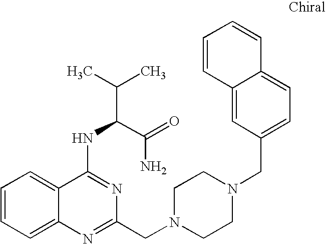 | 482.6 | 483.3 |
| 21-38 | 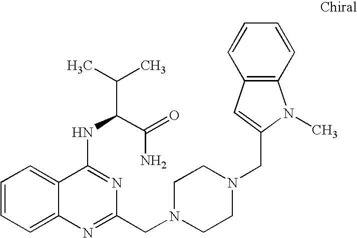 | 485.6 | 486.3 |
| 21-39 | 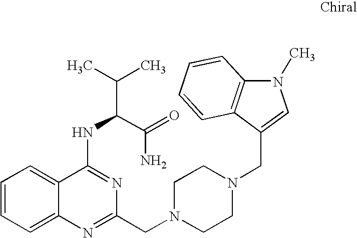 | 485.6 | 486.3 |
| 21-40 | 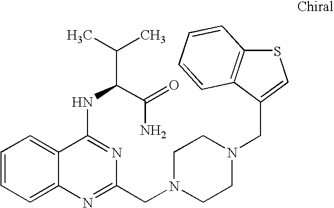 | 488.7 | 489.3 |
| 21-41 | 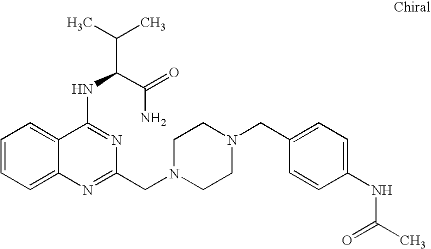 | 489.6 | 490.3 |
| 21-42 | 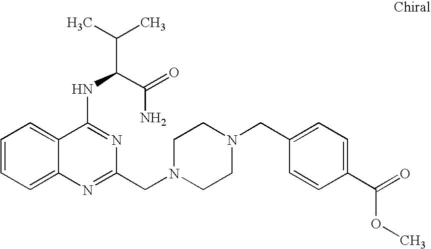 | 490.6 | 491.3 |
| 21-43 | 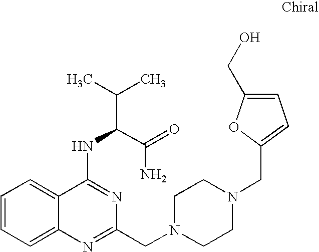 | 452.6 | 453.2 |
| 21-44 | 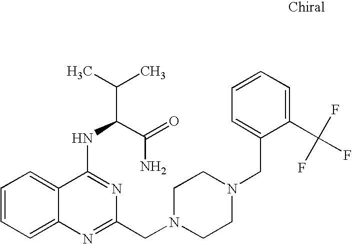 | 500.6 | 501.3 |
| 21-45 | 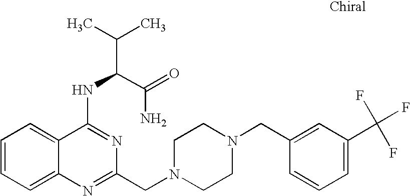 | 500.6 | 501.3 |
| 21-46 | 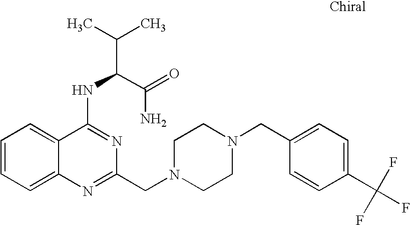 | 500.6 | 501.3 |
| 21-47 | 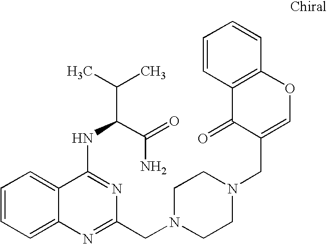 | 500.6 | 501.3 |
| 21-48 | 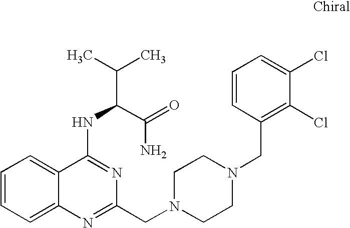 | 501.5 | 501.3 |
| 21-49 | 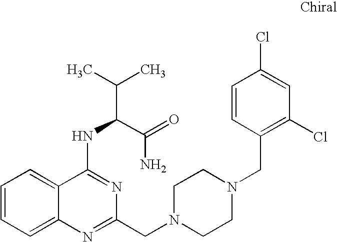 | 501.5 | 501.3 |
| 21-50 | 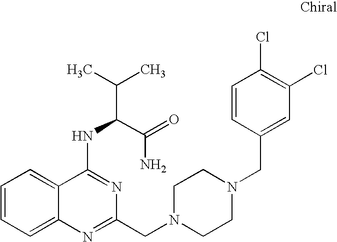 | 501.5 | 501.3 |
| 21-51 | 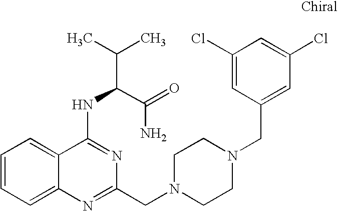 | 501.5 | 501.3 |
| 21-52 | 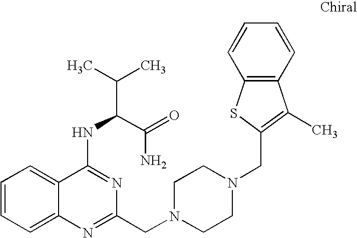 | 502.7 | 503.3 |
| 21-53 | 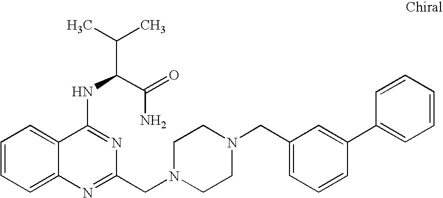 | 508.7 | 509.3 |
| 21-54 | 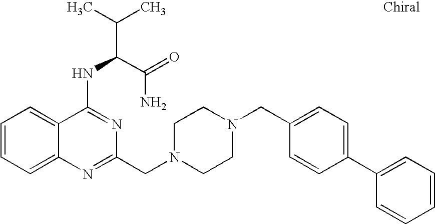 | 508.7 | 509.3 |
| 21-55 | 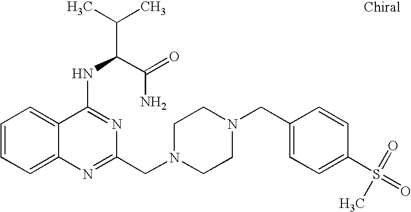 | 510.7 | 511.3 |
| 21-56 | 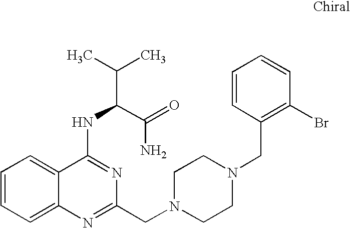 | 511.5 | 513.3 |
| 21-57 | 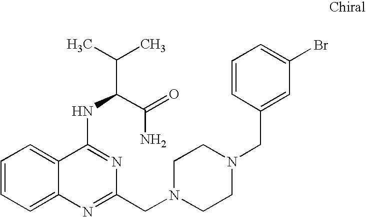 | 511.5 | 513.3 |
| 21-58 | 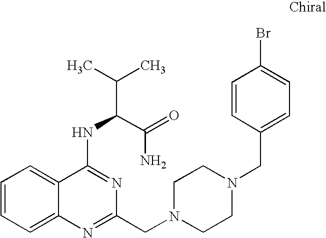 | 511.5 | 513.3 |
| 21-59 | 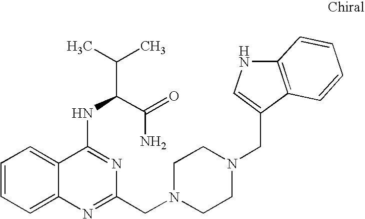 | 471.6 | 472.3 |
| 21-60 | 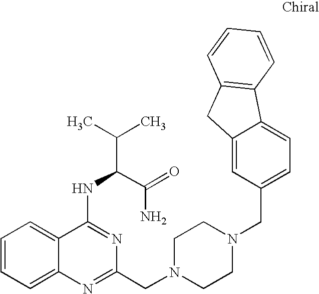 | 520.7 | 521.3 |
| 21-61 | 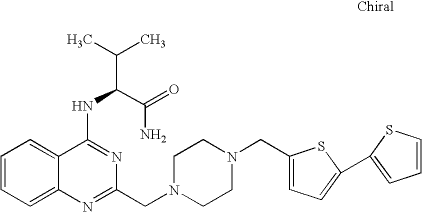 | 520.7 | 521.3 |
| 21-62 | 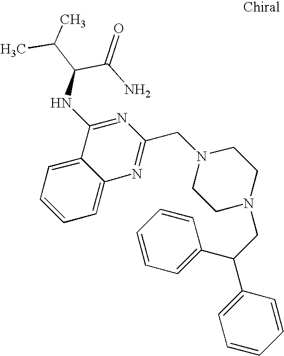 | 522.7 | 523.3 |
| 21-63 | 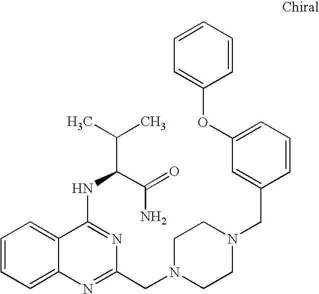 | 524.7 | 525.3 |
| 21-64 | 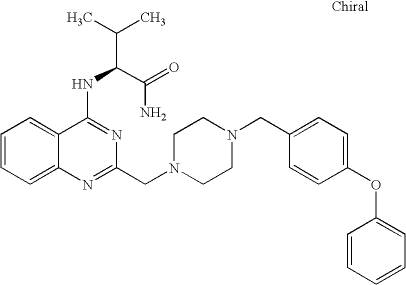 | 524.7 | 525.3 |
| 21-65 | 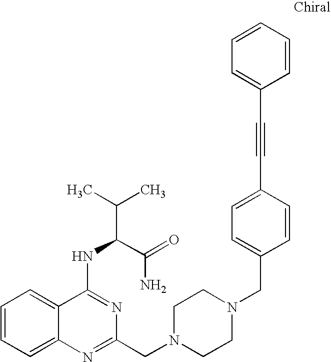 | 532.7 | 533.3 |
| 21-66 | 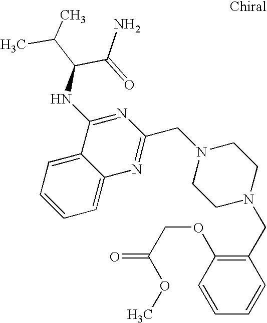 | 520.6 | 521.3 |
| 21-67 | 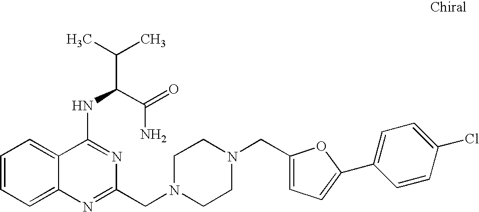 | 533.1 | 533.3 |
| 21-68 | 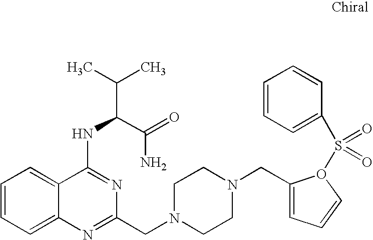 | 561.7 | 562.3 |
| 21-69 | 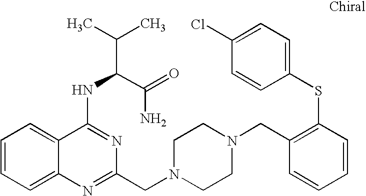 | 575.2 | 575.3 |
| 21-70 | 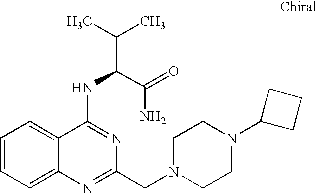 | 396.5 | 397.2 |
| 21-71 | 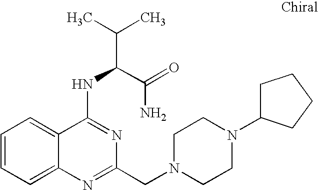 | 410.6 | 411.2 |
| 21-72 | 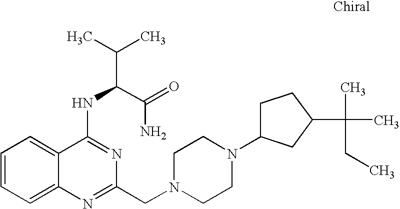 | 480.7 | 481.3 |
| 21-73 | 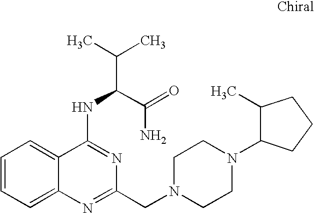 | 424.6 | 425.2 |
| 21-74 | 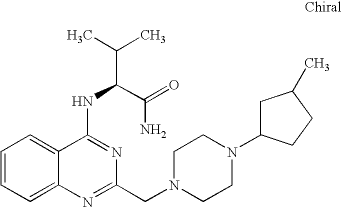 | 424.6 | 425.2 |
| 21-75 | 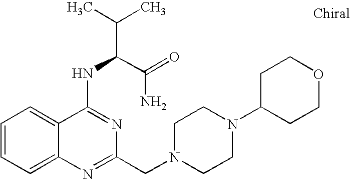 | 426.6 | 427.2 |
| 21-76 | 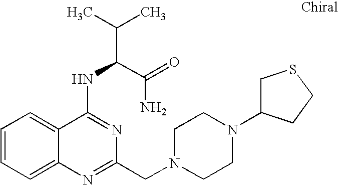 | 428.6 | 429.2 |
| 21-77 | 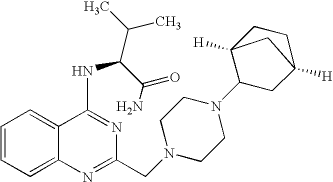 | 436.6 | 437.2 |
| 21-78 | 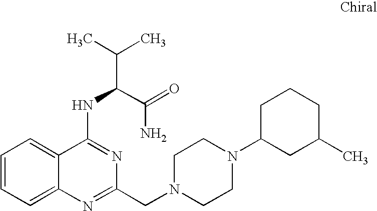 | 438.6 | 439.2 |
| 21-79 | 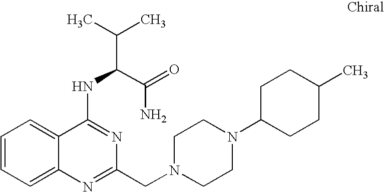 | 438.6 | 439.2 |
| 21-80 | 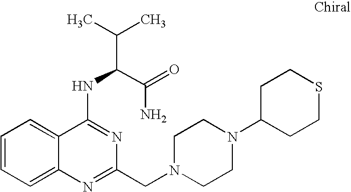 | 442.6 | 443.2 |
| 21-81 | 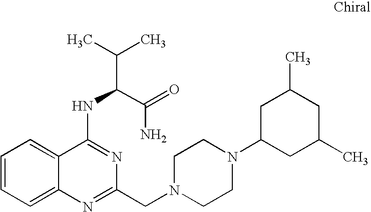 | 452.6 | 453.2 |
| 21-82 | 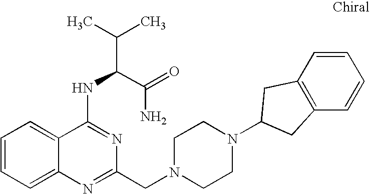 | 458.6 | 459.3 |
| 21-83 | 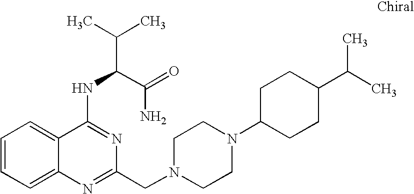 | 466.7 | 467.3 |
| 21-84 | 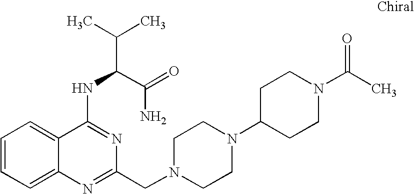 | 467.6 | 468.3 |
| 21-85 | 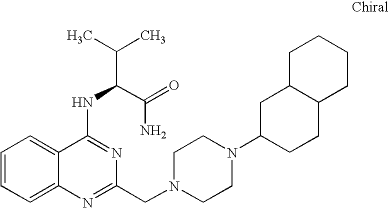 | 478.7 | 479.3 |
| 21-86 | 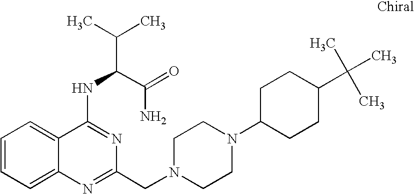 | 480.7 | 481.3 |
| 21-87 | 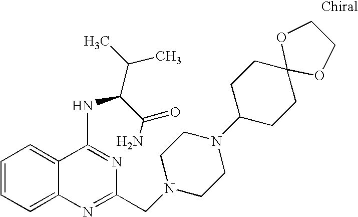 | 482.6 | 483.3 |
| 21-88 | 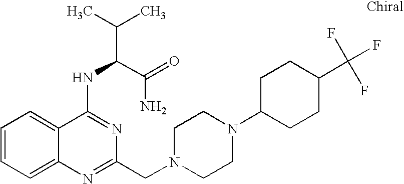 | 492.6 | 493.3 |
| 21-89 | 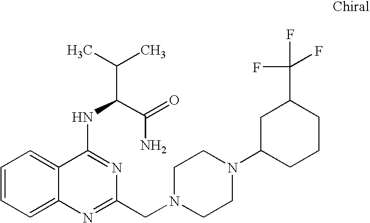 | 492.6 | 493.3 |
| 21-90 | 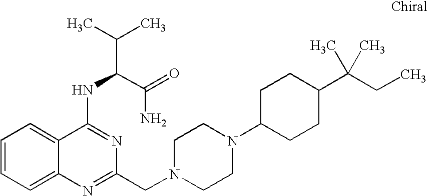 | 494.7 | 495.3 |
| 21-91 | 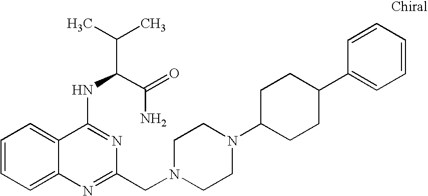 | 500.7 | 501.3 |
| 21-92 | 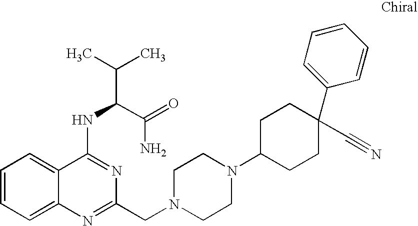 | 525.7 | 526.3 |
| 21-93 | 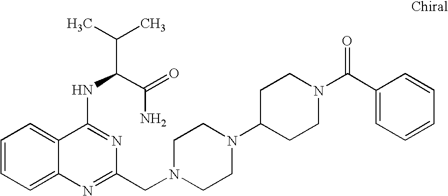 | 529.7 | 530.3 |
| 21-94 | 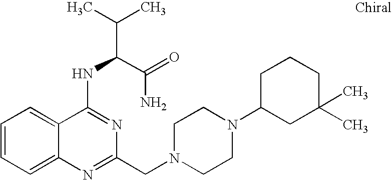 | 452.6 | 453.2 |
| 21-95 | 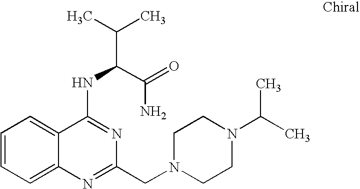 | 384.5 | 385.2 |
| 21-96 | 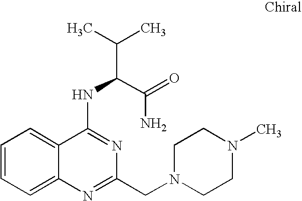 | 356.5 | 357.2 |
| 21-97 | 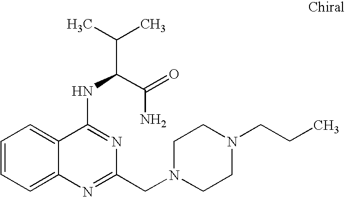 | 384.5 | 385.2 |

### Example 22

### Reaction of 2(S)-(−)-[2-(PIPERAZIN-1-YLMETHYL)QUINAZOLIN-4-YLAMINO]-3-METHYLBUTYRAMIDE with an ACID LIBRARY

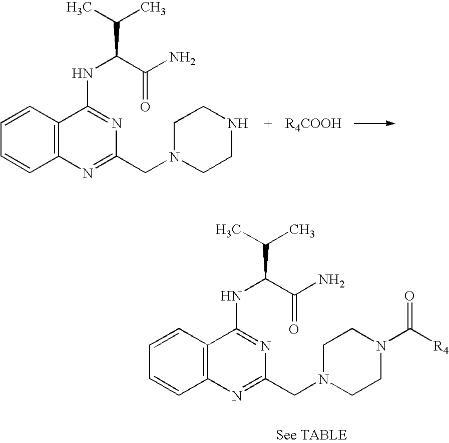

A stock solution of 2(S)-(−)-[2-(piperazin-1-ylmethyl)quinazolin-4-ylamino]-3-methylbutyramide (1 mL, 0.0233 mmoles) (prepared as described in Preparative Example 22 above) in THF was added to the tubes in three 24 tube Bohdan Miniblocks. PS-EDC resin (41 mg, 0.0583 mmoles) was added to each tube, followed by a stock solution of HOBT in THF (0.5 mL, 0.0350 mmoles). 1M stock solutions of each of the individual acids (R₄COOH) (0.03 mL, 0.0303 mmoles) were added to the tubes and the Miniblocks were sealed and shaken at 25° C. for 21 h. PS-Isocyanate resin (3 equivalents, 0.0699 mmoles) was added to each tube, followed by PS-Trisamine resin (6 equivalents, 0.14 mmoles) and the Miniblocks were shaken at 25° C. for 4 h. The tubes were drained and the resin was washed with THF (2×1 mL), shaking for 5 min each time. The combined filtrates from each tube, were evaporated to dryness overnight, on a Speedvac concentrator. The resulting samples were evaluated by LCMS and those that were >70% pure are listed in table below.

### Examples 22-1 Through 22-71

| EXAMPLE | STRUCTURE | MW | LCMS m/z |
|---|---|---|---|
| 22-1 | 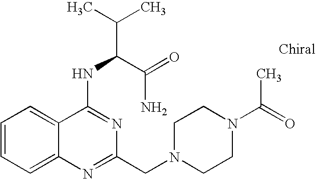 | 384.5 | 385.1 |
| 22-2 | 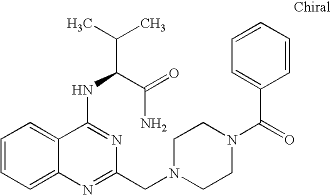 | 446.6 | 447.1 |
| 22-3 | 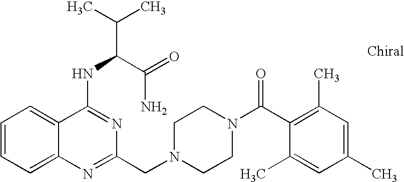 | 488.6 | 489.1 |
| 22-4 | 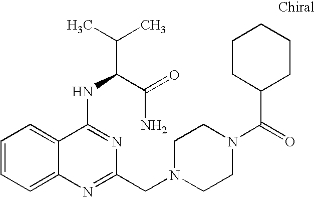 | 452.6 | 453.1 |
| 22-5 | 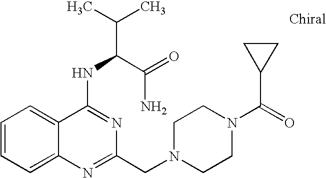 | 410.5 | 411.2 |
| 22-6 | 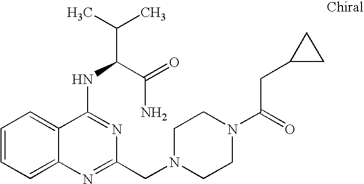 | 424.6 | 425.2 |
| 22-7 | 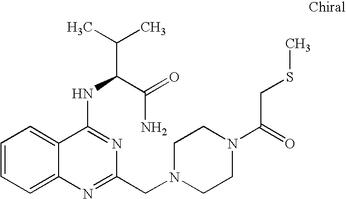 | 430.6 | 431.2 |
| 22-8 | 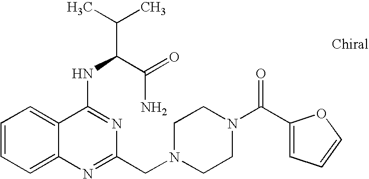 | 436.5 | 437.2 |
| 22-9 | 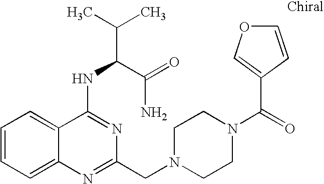 | 436.5 | 437.2 |
| 22-10 | 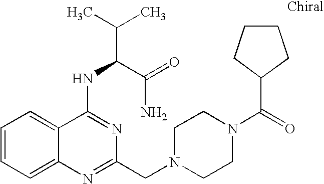 | 438.6 | 439.2 |
| 22-11 | 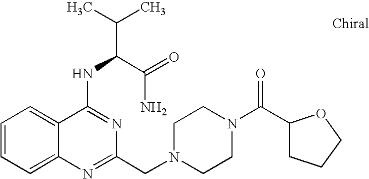 | 440.6 | 441.2 |
| 22-12 | 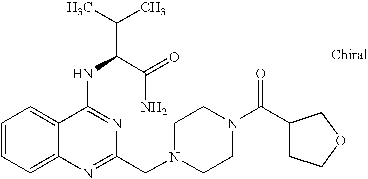 | 440.6 | 441.2 |
| 22-13 | 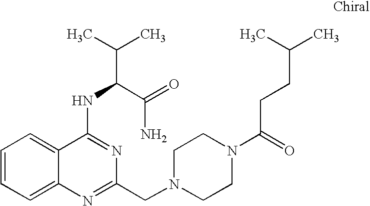 | 440.6 | 441.2 |
| 22-14 | 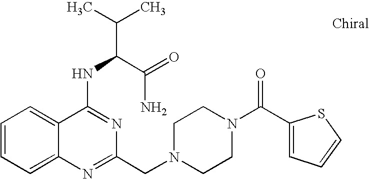 | 452.6 | 453.2 |
| 22-15 | 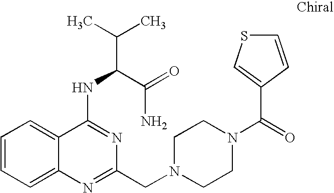 | 452.6 | 453.2 |
| 22-16 | 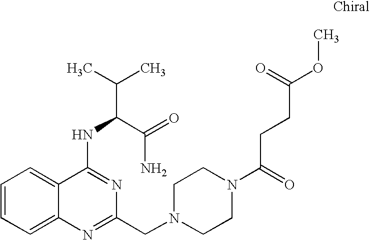 | 456.5 | 4573 |
| 22-17 | 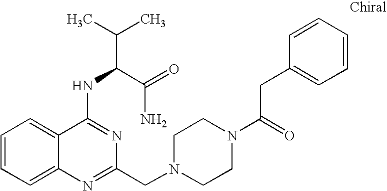 | 460.6 | 461.3 |
| 22-18 | 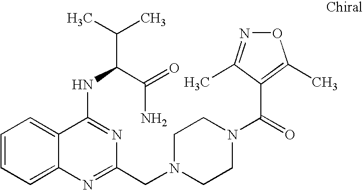 | 465.6 | 466.3 |
| 22-19 | 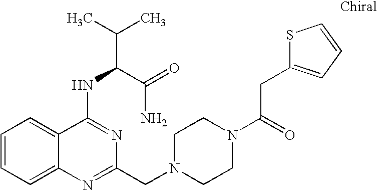 | 466.6 | 467.3 |
| 22-20 | 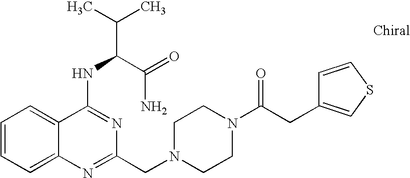 | 466.6 | 467.3 |
| 22-21 | 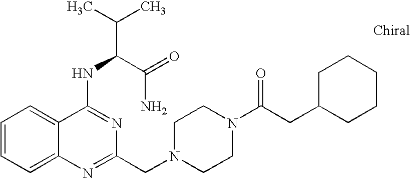 | 466.6 | 467.3 |
| 22-22 | 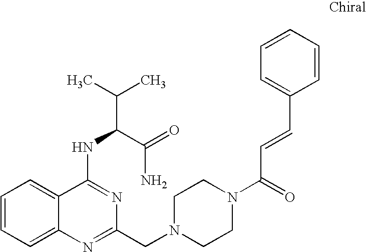 | 472.6 | 473.3 |
| 22-23 | 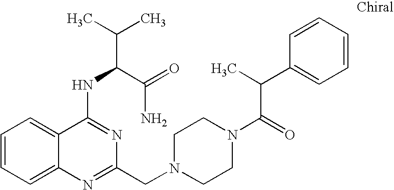 | 474.6 | 475.3 |
| 22-24 | 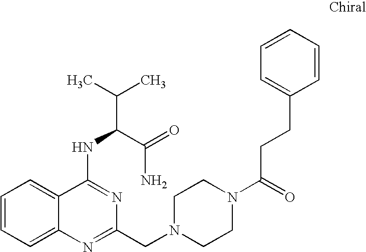 | 474.6 | 475.3 |
| 22-25 | 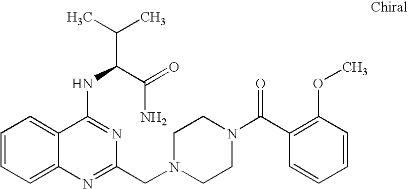 | 476.6 | 477.3 |
| 22-26 | 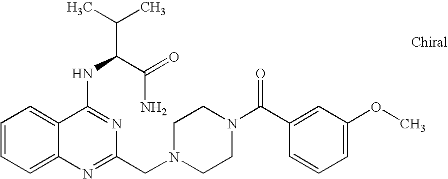 | 476.6 | 477.3 |
| 22-27 | 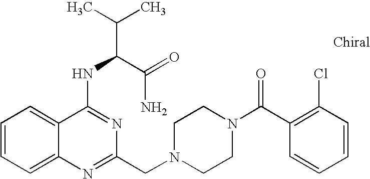 | 481.0 | 481.3 |
| 22-28 | 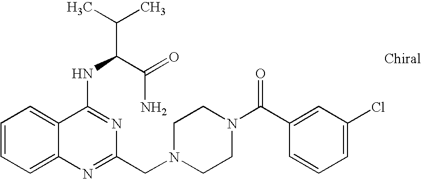 | 481.0 | 481.3 |
| 22-29 | 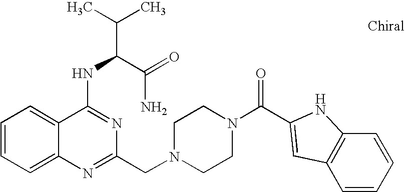 | 485.6 | 486.3 |
| 22-30 | 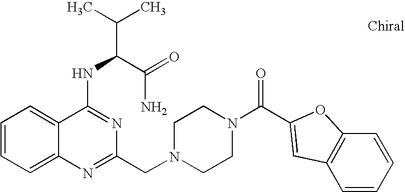 | 486.6 | 487.3 |
| 22-31 | 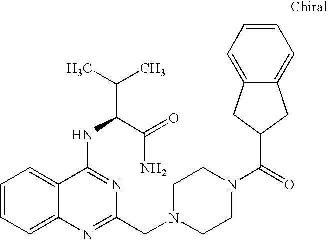 | 486.6 | 487.3 |
| 22-32 | 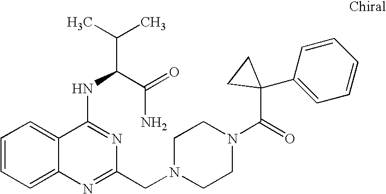 | 486.6 | 487.3 |
| 22-33 | 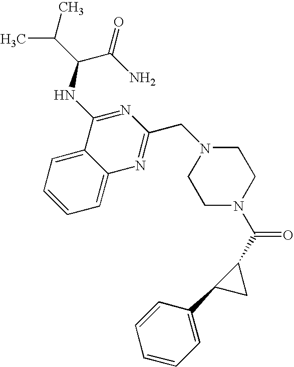 | 486.6 | 487.3 |
| 22-34 | 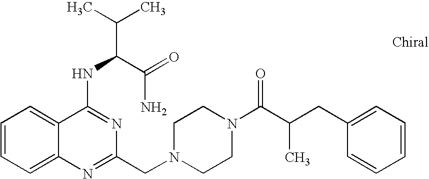 | 488.6 | 489.3 |
| 22-35 | 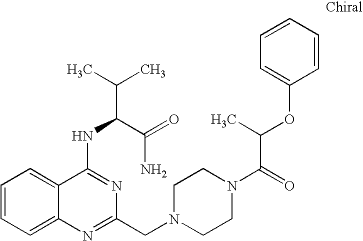 | 490.6 | 491.3 |
| 22-36 | 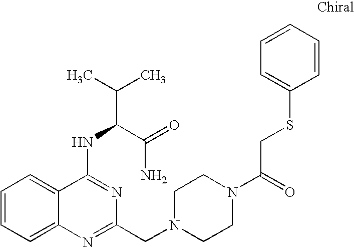 | 492.6 | 493.3 |
| 22-37 | 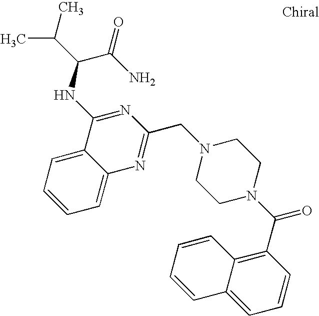 | 496.6 | 497.3 |
| 22-38 | 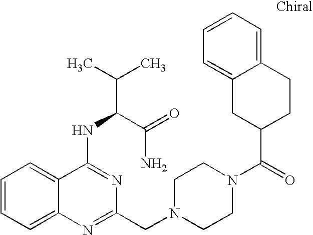 | 500.6 | 501.3 |
| 22-39 | 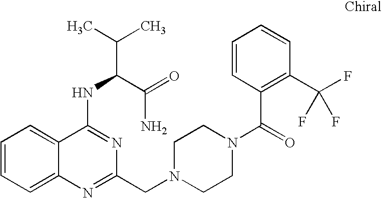 | 514.6 | 515.3 |
| 22-40 | 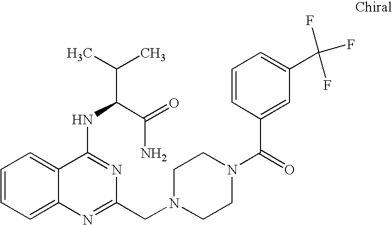 | 514.6 | 515.3 |
| 22-41 | 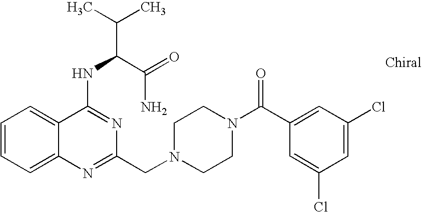 | 515.4 | 515.3 |
| 22-42 | 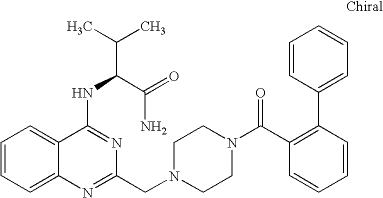 | 522.7 | 523.3 |
| 22-43 | 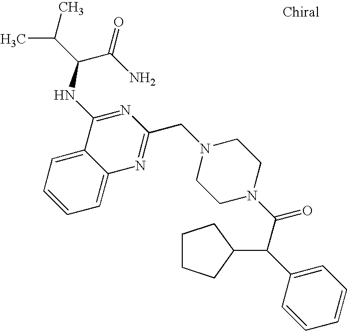 | 528.7 | 529.3 |
| 22-44 | 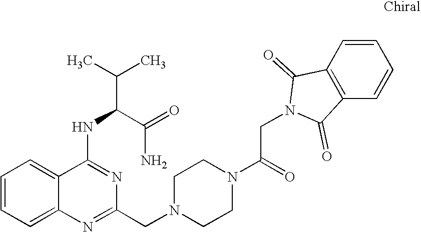 | 529.6 | 530.3 |
| 22-45 | 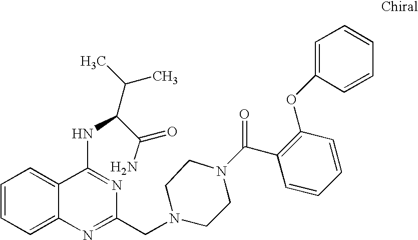 | 538.7 | 539.3 |
| 22-46 | 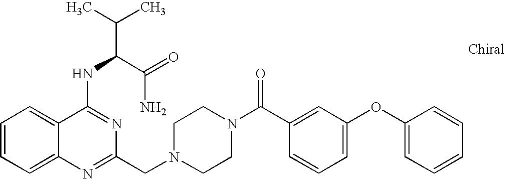 | 538.7 | 539.3 |
| 22-47 | 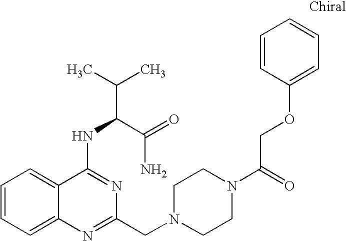 | 476.6 | 477.3 |
| 22-48 | 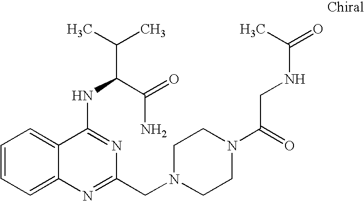 | 441.5 | 442.2 |
| 22-49 | 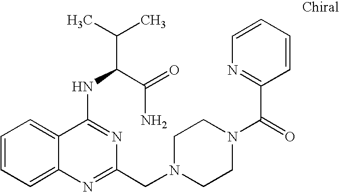 | 447.5 | 448.2 |
| 22-50 | 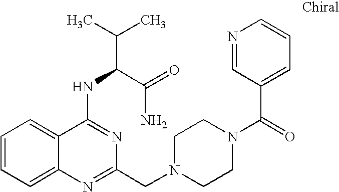 | 447.5 | 448.2 |
| 22-51 | 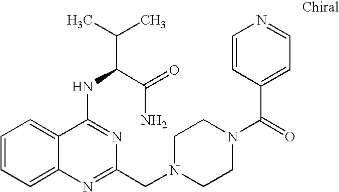 | 447.5 | 448.2 |
| 22-52 | 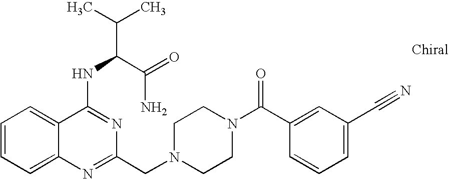 | 471.6 | 472.3 |
| 22-53 | 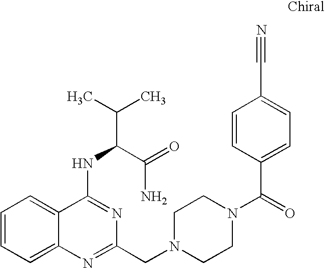 | 471.6 | 472.3 |
| 22-54 | 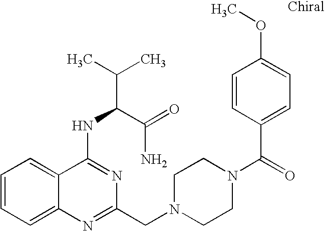 | 476.6 | 477.3 |
| 22-55 | 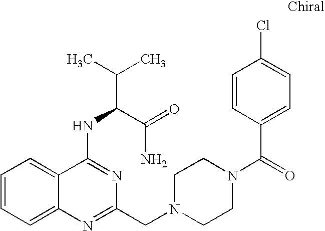 | 481.0 | 481.3 |
| 22-56 | 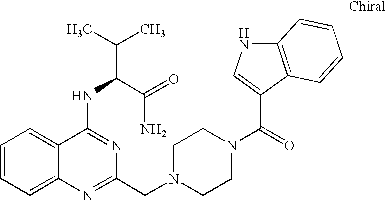 | 485.6 | 486.3 |
| 22-57 | 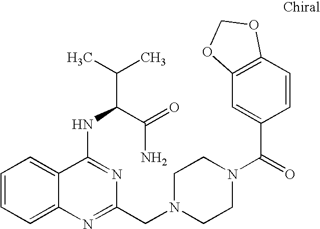 | 490.6 | 491.3 |
| 22-58 | 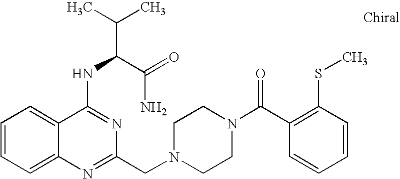 | 492.6 | 493.3 |
| 22-59 | 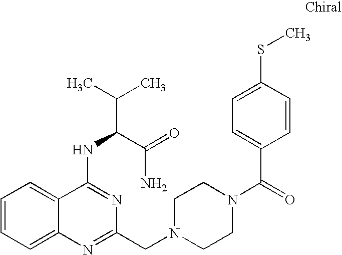 | 492.6 | 493.3 |
| 22-60 | 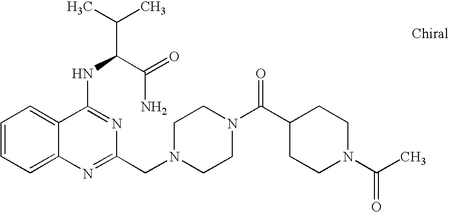 | 495.6 | 496.3 |
| 22-61 | 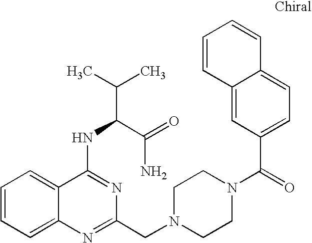 | 496.6 | 497.3 |
| 22-62 | 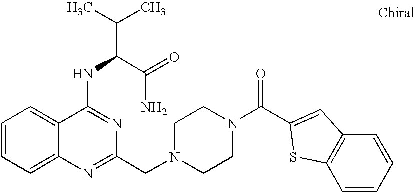 | 502.6 | 503.3 |
| 22-63 | 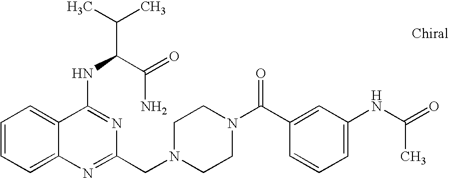 | 503.6 | 504.3 |
| 22-64 | 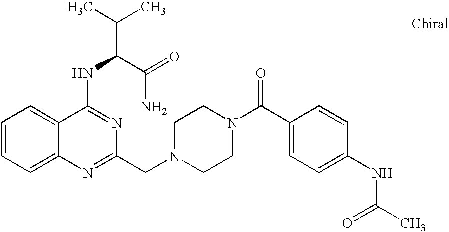 | 503.6 | 504.3 |
| 22-65 | 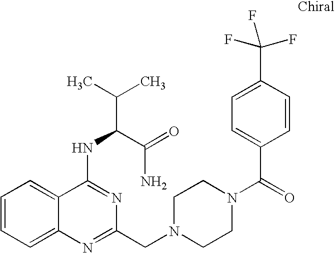 | 514.6 | 515.3 |
| 22-66 | 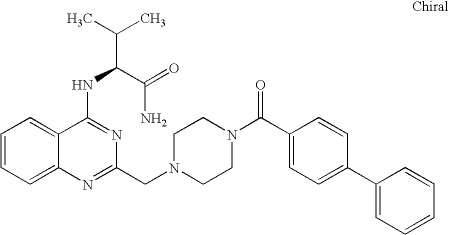 | 522.7 | 523.3 |
| 22-67 | 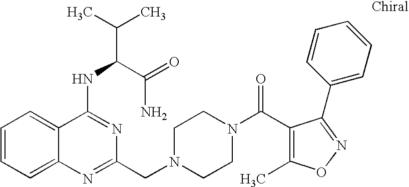 | 527.6 | 528.3 |
| 22-68 | 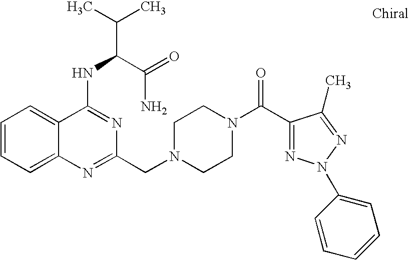 | 527.6 | 528.3 |
| 22-69 | 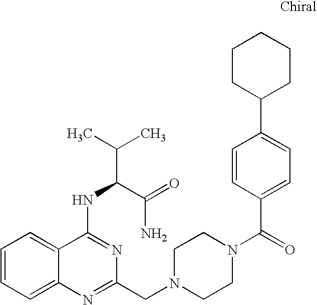 | 528.7 | 529.3 |
| 22-70 | 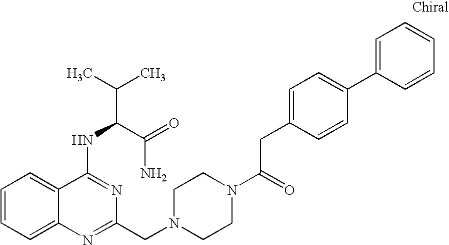 | 536.7 | 537.3 |
| 22-71 | 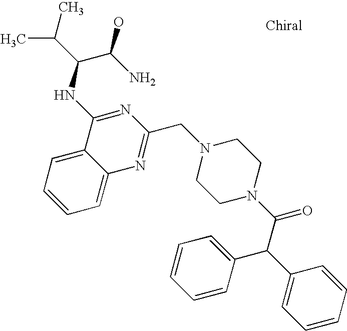 | 536.7 | 537.3 |

### Example 23

### Reaction of 2(S)-(−)-[2-(PIPERAZIN-1-YLMETHYL)QUINAZOLIN-4-YLAMINO]-3-METHYLBUTYRAMIDE with a SULFONYL CHLORIDE LIBRARY

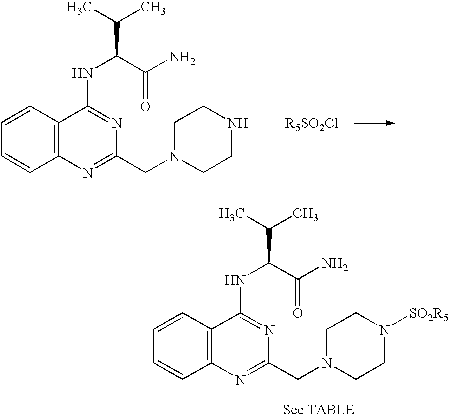

A stock solution of 3-methyl-2(S)-(−)-[2-(piperazin-1-ylmethyl)quinazolin-4-ylamino]butyric acid methyl ester (1 mL, 0.0233 mmoles) (prepared as described in Preparative Example 22 above) in THF was added to the tubes in four 24 tube Bohdan Miniblocks. PS-DIEA resin (3 equivalents, 0.0699 mmoles) was added to all of the tubes, followed by a 1M solution of each of the sulfonyl chlorides (R₅SO₂Cl) (1.5 equivalents, 0.0350 mmoles) in THF. Additional THF (0.5 mL) was added to each tube and the Miniblocks were sealed and shaken at 25° C. for 22 h. PS-Isocyanate resin (3 equivalents, 0.0699 mmoles) was added to each tube, followed by PS-Trisamine resin (6 equivalents, 0.14 mmoles) and the Miniblocks were shaken at 25° C. for 4 h. The tubes were drained and the resin was washed with THF (2×1 mL), shaking for 5 min each time. The filtrates from each tube were combined and evaporated to dryness overnight, on a Speedvac concentrator. The resulting samples were evaluated by LCMS and those that were >70% pure are listed in the table below.

### Examples 23-1 Through 23-77

| EXAMPLE | STRUCTURE | MW | LCMS m/z |
|---|---|---|---|
| 23-1 | 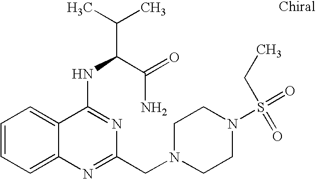 | 434.6 | 435.2 |
| 23-2 | 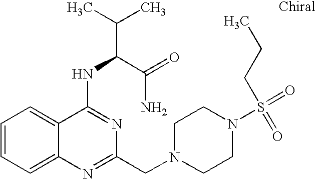 | 448.6 | 449.2 |
| 23-3 | 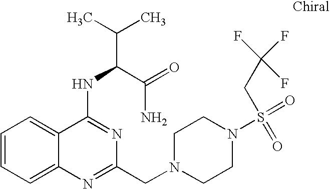 | 488.5 | 489.3 |
| 23-4 | 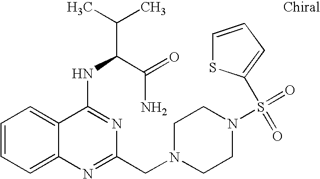 | 488.6 | 489.3 |
| 23-5 | 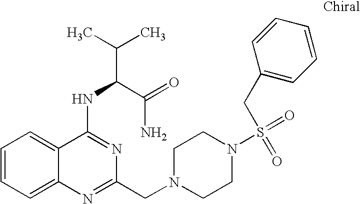 | 496.6 | 497.3 |
| 23-6 | 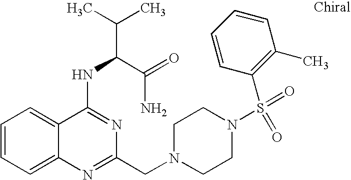 | 496.6 | 497.3 |
| 23-7 | 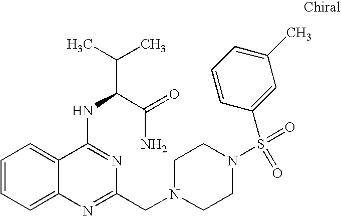 | 496.6 | 497.3 |
| 23-8 | 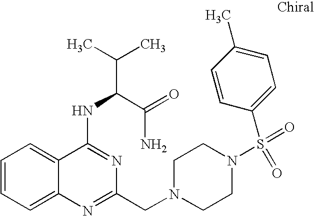 | 496.6 | 497.3 |
| 23-9 | 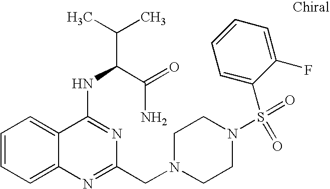 | 500.6 | 501.3 |
| 23-10 | 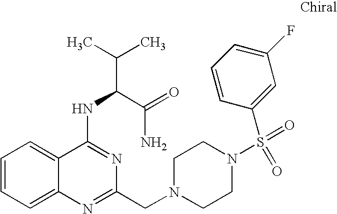 | 500.6 | 501.3 |
| 23-11 | 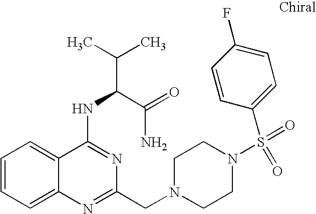 | 500.6 | 501.3 |
| 23-12 | 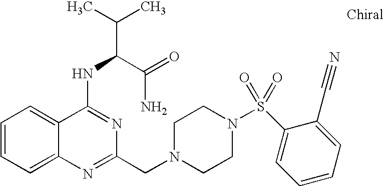 | 507.6 | 508.3 |
| 23-13 | 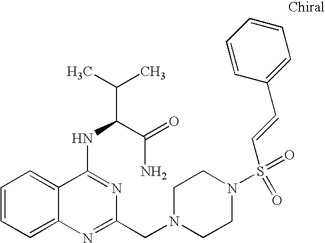 | 508.6 | 509.3 |
| 23-14 | 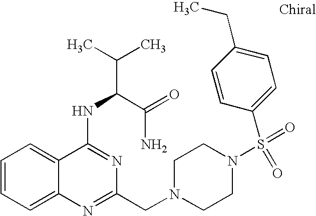 | 510.7 | 511.3 |
| 23-15 | 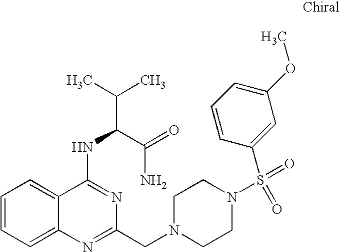 | 512.6 | 513.3 |
| 23-16 | 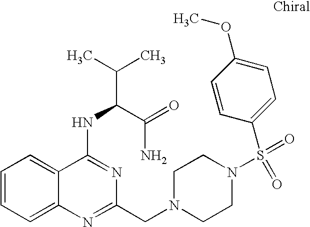 | 512.6 | 513.3 |
| 23-17 | 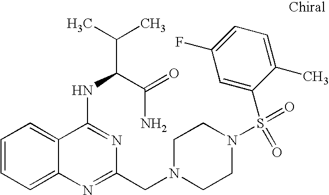 | 514.6 | 515.3 |
| 23-18 | 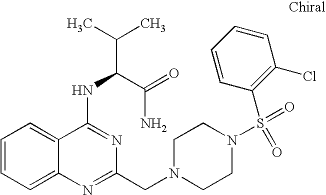 | 517.1 | 517.3 |
| 23-19 | 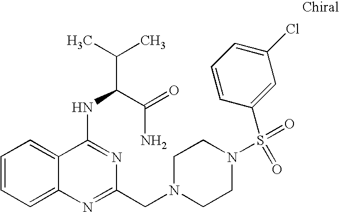 | 517.1 | 517.3 |
| 23-20 | 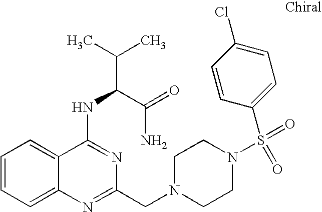 | 517.1 | 517.3 |
| 23-21 | 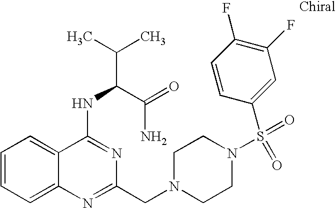 | 518.6 | 519.3 |
| 23-22 | 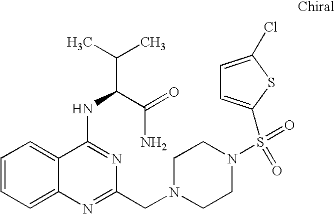 | 523.1 | 523.3 |
| 23-23 | 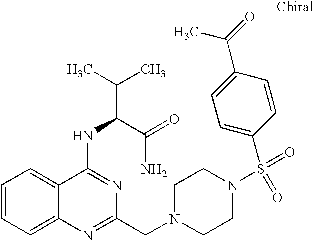 | 524.6 | 525.3 |
| 23-24 | 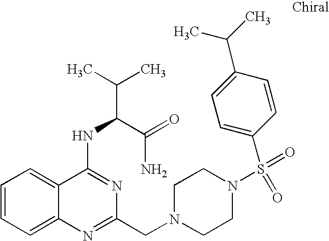 | 524.7 | 525.3 |
| 23-25 | 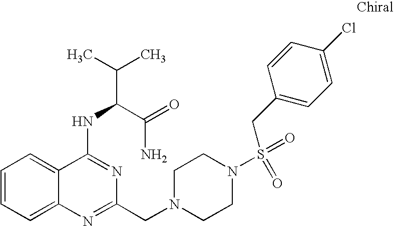 | 531.1 | 531.3 |
| 23-26 | 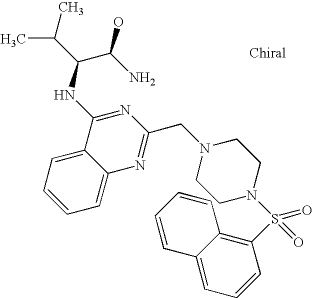 | 532.7 | 533.3 |
| 23-27 | 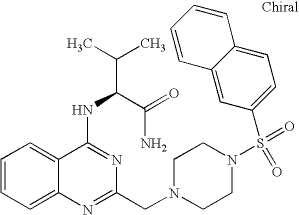 | 532.7 | 533.3 |
| 23-28 | 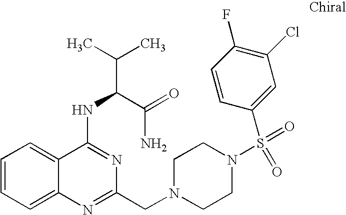 | 535.0 | 535.3 |
| 23-29 | 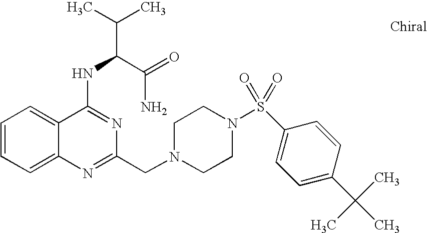 | 538.7 | 539.3 |
| 23-30 | 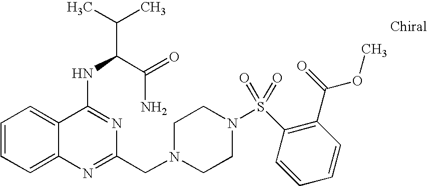 | 540.6 | 541.3 |
| 23-31 | 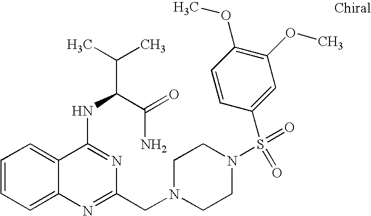 | 542.7 | 543.3 |
| 23-32 | 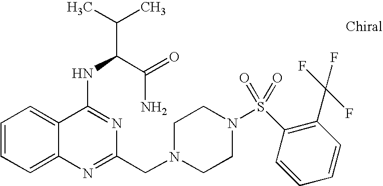 | 550.6 | 551.3 |
| 23-33 | 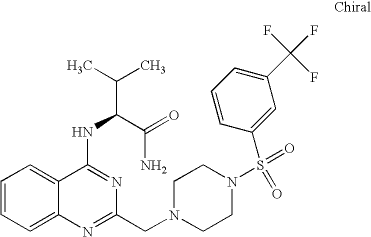 | 550.6 | 551.3 |
| 23-34 | 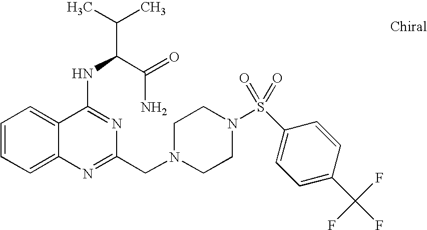 | 550.6 | 551.3 |
| 23-35 | 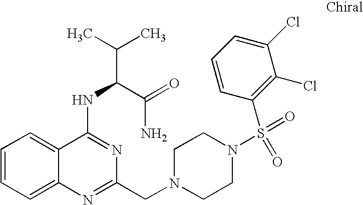 | 551.5 | 551.3 |
| 23-36 | 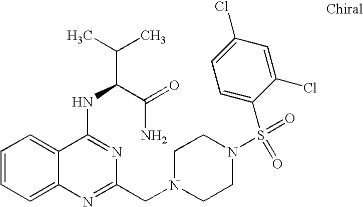 | 551.5 | 551.3 |
| 23-37 | 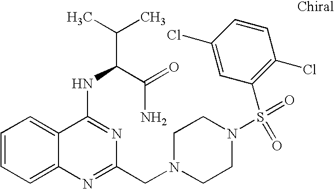 | 551.5 | 551.3 |
| 23-38 | 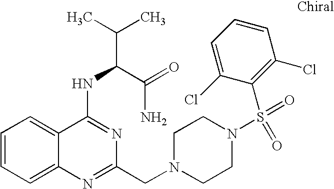 | 551.5 | 551.3 |
| 23-39 | 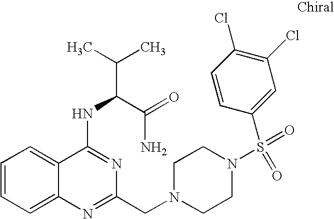 | 551.5 | 551.3 |
| 23-40 | 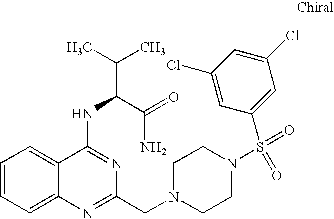 | 551.5 | 551.3 |
| 23-41 | 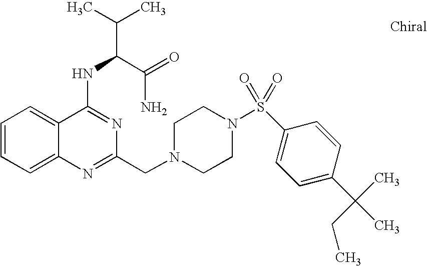 | 552.7 | 553.3 |
| 23-42 | 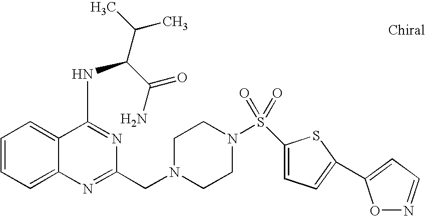 | 555.7 | 556.3 |
| 23-43 | 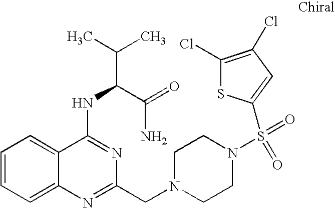 | 557.5 | 557.3 |
| 23-44 | 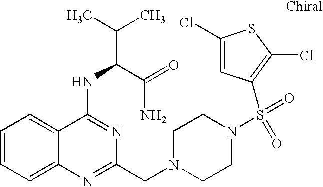 | 557.5 | 557.3 |
| 23-45 | 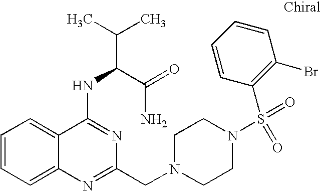 | 561.5 | 563.3 |
| 23-46 | 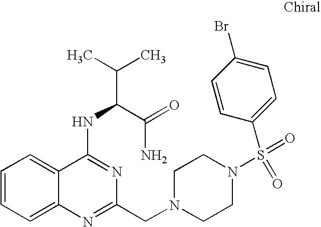 | 561.5 | 563.3 |
| 23-47 | 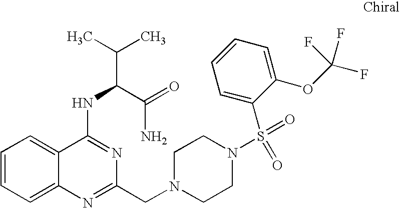 | 566.6 | 567.3 |
| 23-48 | 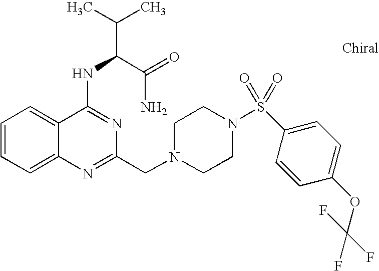 | 566.6 | 567.3 |
| 23-49 | 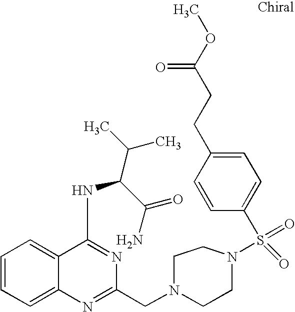 | 568.7 | 569.3 |
| 23-50 | 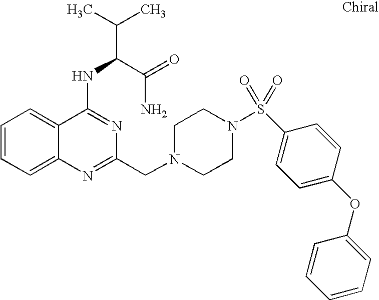 | 574.7 | 575.3 |
| 23-51 | 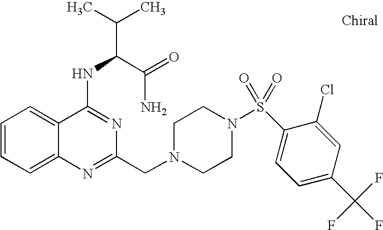 | 585.1 | 585.3 |
| 23-52 | 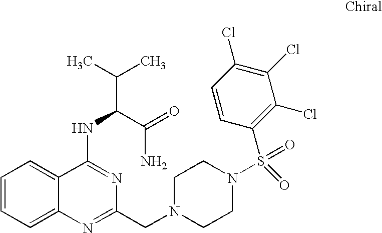 | 585.9 | 587.3 |
| 23-53 | 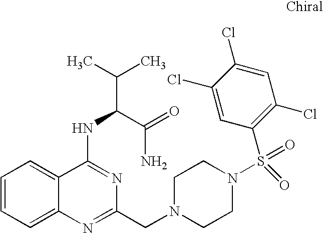 | 585.9 | 587.3 |
| 23-54 | 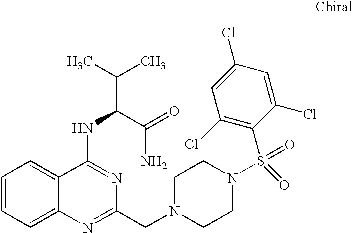 | 585.9 | 587.3 |
| 23-55 | 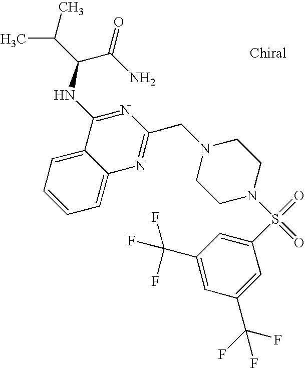 | 618.6 | 619.3 |
| 23-56 | 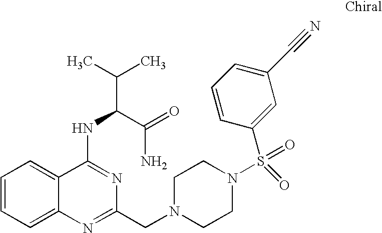 | 507.6 | 508.3 |
| 23-57 | 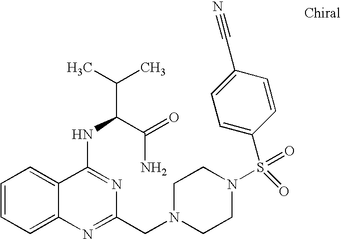 | 507.6 | 508.3 |
| 23-58 | 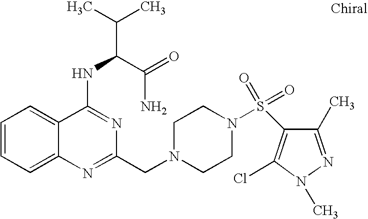 | 535.1 | 535.3 |
| 23-59 | 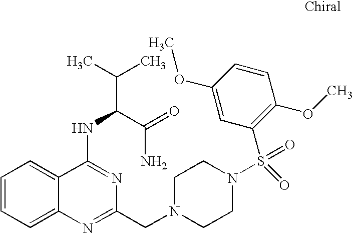 | 542.7 | 5433 |
| 23-60 | 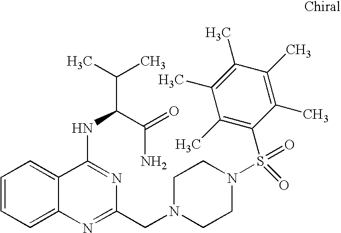 | 552.7 | 553.3 |
| 23-61 | 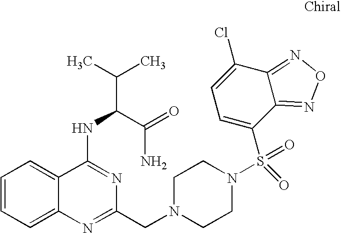 | 559.1 | 559.3 |
| 23-62 | 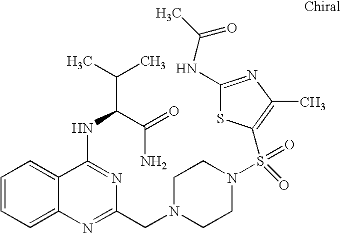 | 560.7 | 561.3 |
| 23-63 | 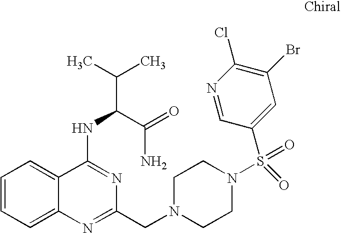 | 596.9 | 598.3 |
| 23-64 | 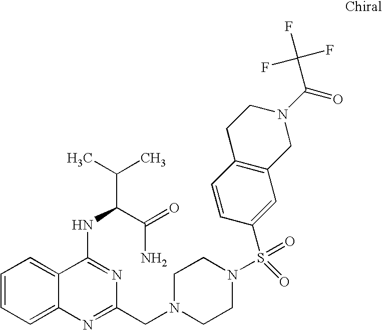 | 633.7 | 634.3 |
| 23-65 | 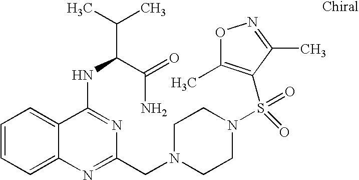 | 501.6 | 502.3 |
| 23-66 | 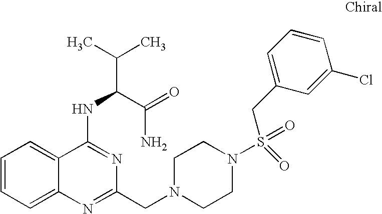 | 531.1 | 531.3 |
| 23-67 | 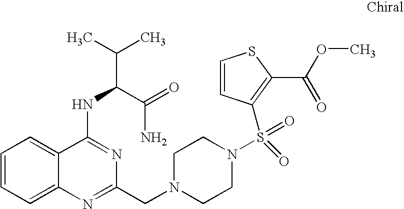 | 546.7 | 547.3 |
| 23-68 | 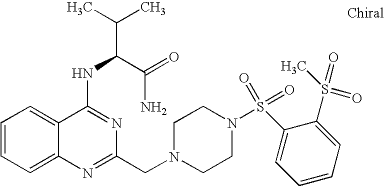 | 560.7 | 561.3 |
| 23-69 | 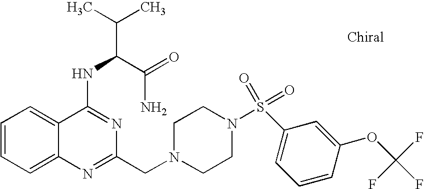 | 566.6 | 567.3 |
| 23-70 | 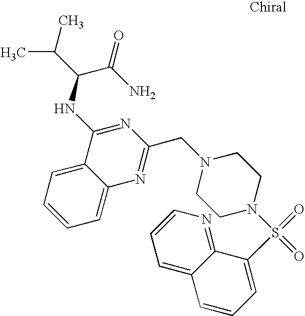 | 533.7 | 534.3 |
| 23-71 | 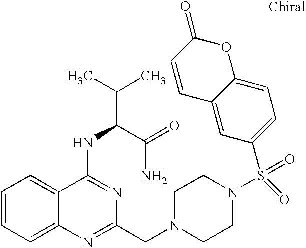 | 550.6 | 551.3 |
| 23-72 | 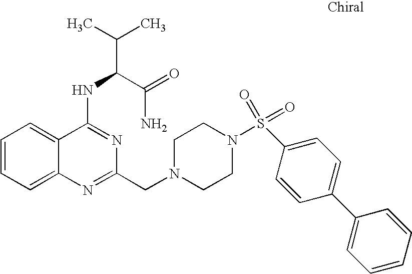 | 558.7 | 559.3 |
| 23-73 | 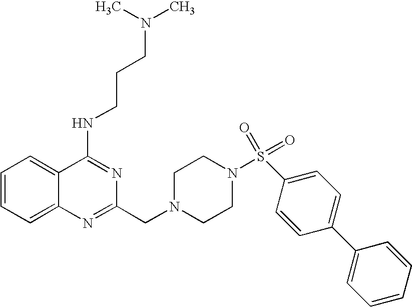 | 544.7 | 545.3 |
| 23-74 | 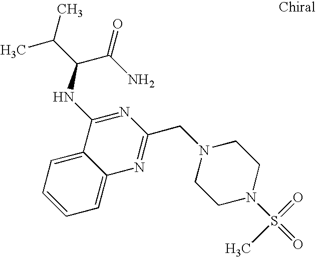 | 420.5 | 421.2 |
| 23-75 | 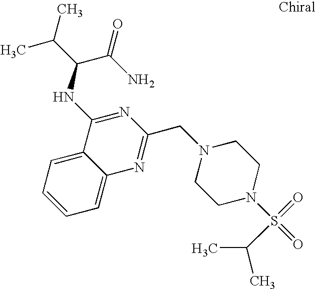 | 448.6 | 449.2 |
| 23-76 | 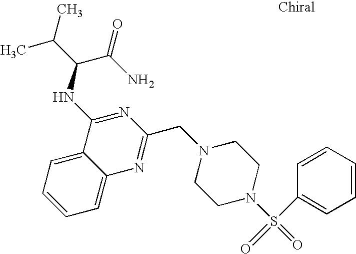 | 482.6 | 483.3 |
| 23-77 | 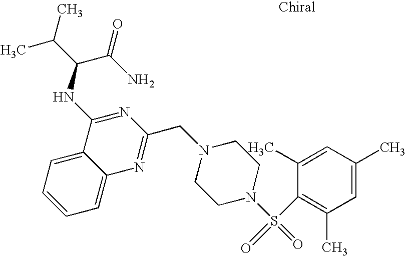 | 524.7 | 525.3 |

### Example 24

### Reaction of 2(S)-(−)-[2-(PIPERAZIN-1-YLMETHYL)QUINAZOLIN-4-YLAMINO]-3-METHYLBUTYRAMIDE with an ISOCYANATE LIBRARY

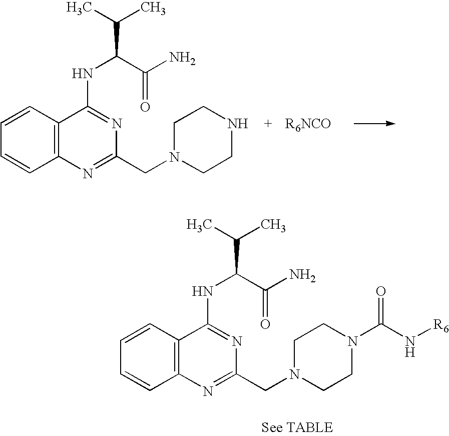

A stock solution of 2(S)-(−)-[2-(piperazin-1-ylmethyl)quinazolin-4-ylamino]-3-methylbutyramide (1 mL, 0.0233 mmoles) (prepared as described in Preparative Example 22 above) in THF was added to the tubes in three 24 tube Bohdan Miniblocks. A 1M stock solution of each of the isocyanates (R₆NCO) in THF (0.05 mL, 0.0466 mmoles) was added to each tube. PS-Isocyanate resin (3 equivalents, 0.0699 mmoles) was added to each tube, followed by PS-Trisamine resin (6 equivalents, 0.14 mmoles) and the Miniblocks were shaken at 25° C. for 18 h. The blocks were drained onto MP-TsOH resin (4 equivalents, 0.0932 mmoles) and the PS-Trisamine resins were washed with THF. The Miniblocks were shaken at 25° C. for 4 h and then drained and the resin was washed with dichloromethane and the filtrates were discarded. Ammonia in methanol (2N, 2 mL) was added to each tube and the Miniblocks were shaken at 25° C. for 4 h. The methanol filtrates were collected and the resin was again shaken with ammonia in methanol (2N, 2 mL). The combined filtrates from each tube were evaporated to dryness overnight, on a Speedvac concentrator. The resulting samples were analyzed by LCMS and those that were >70% pure are listed in the table below.

### Examples 24-1 Through 24-49

| EXAMPLE | STRUCTURE | MW | LCMS m/z |
|---|---|---|---|
| 24-1 | 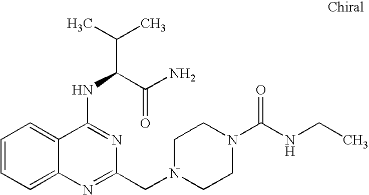 | 413.5 | 414.2 |
| 24-2 | 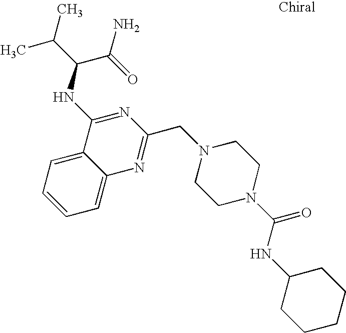 | 467.6 | 468.3 |
| 24-3 | 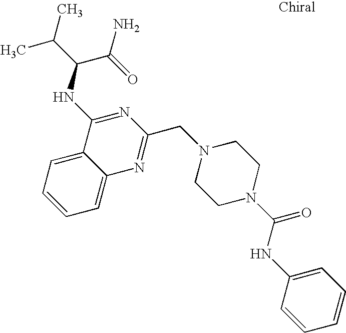 | 461.6 | 462.3 |
| 24-4 | 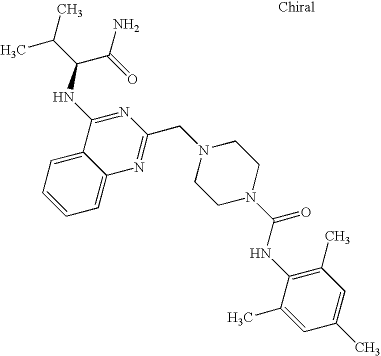 | 503.7 | 504.3 |
| 24-5 | 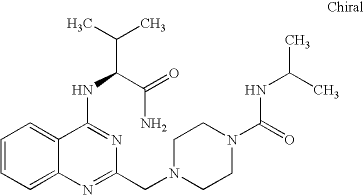 | 427.6 | 428.2 |
| 24-6 | 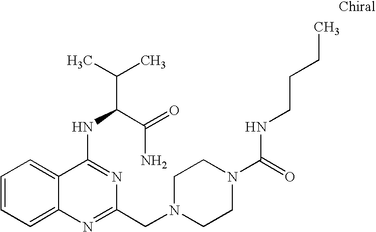 | 441.6 | 442.2 |
| 24-7 | 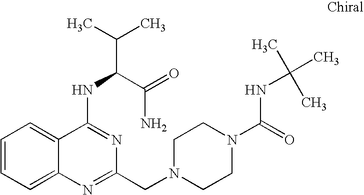 | 441.6 | 442.2 |
| 24-8 | 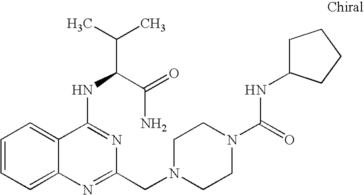 | 453.6 | 454.2 |
| 24-9 | 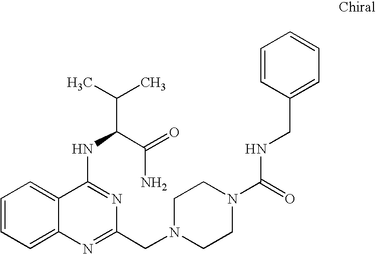 | 475.6 | 476.3 |
| 24-10 | 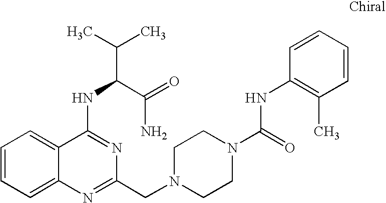 | 475.6 | 476.3 |
| 24-11 | 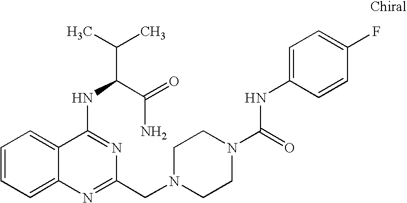 | 479.6 | 480.3 |
| 24-12 | 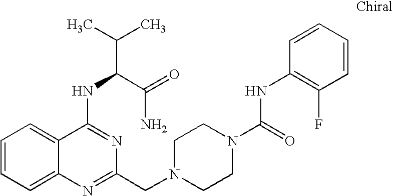 | 479.6 | 480.3 |
| 24-13 | 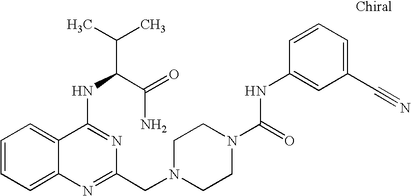 | 486.6 | 487.3 |
| 24-14 | 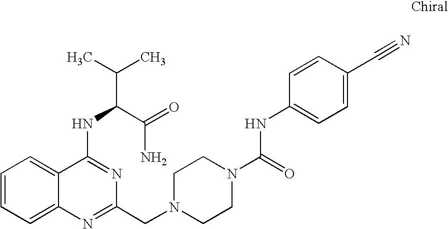 | 486.6 | 487.3 |
| 24-15 | 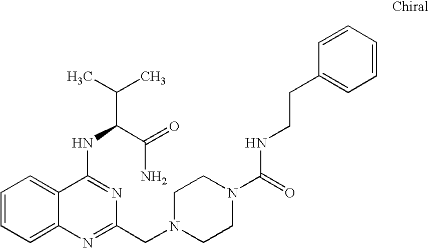 | 489.6 | 490.3 |
| 24-16 | 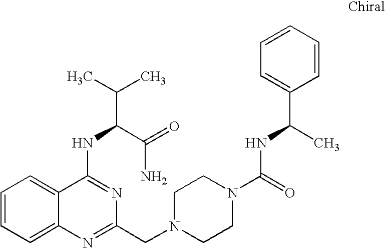 | 489.6 | 490.3 |
| 24-17 | 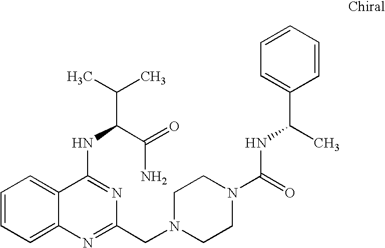 | 489.6 | 490.3 |
| 24-18 | 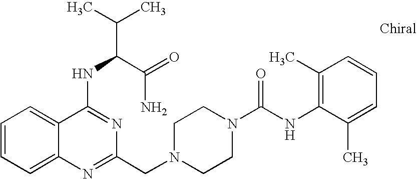 | 489.6 | 490.3 |
| 24-19 | 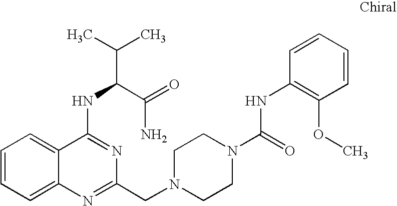 | 491.6 | 492.3 |
| 24-20 | 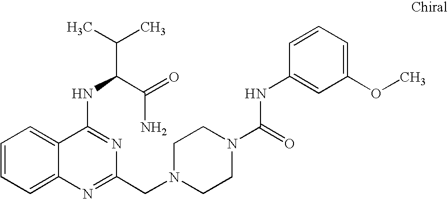 | 491.6 | 492.3 |
| 24-21 | 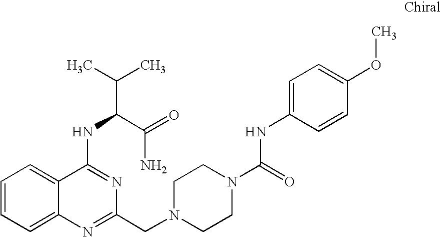 | 491.6 | 492.3 |
| 24-22 | 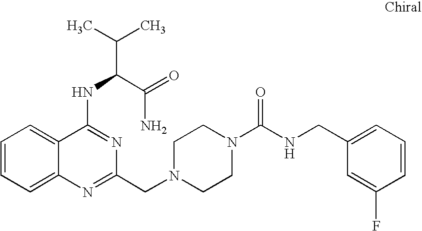 | 493.6 | 494.3 |
| 24-23 | 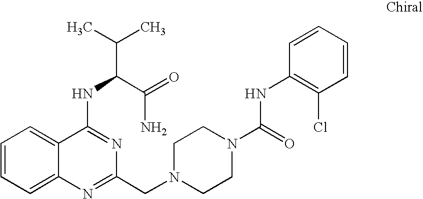 | 496.0 | 496.3 |
| 24-24 | 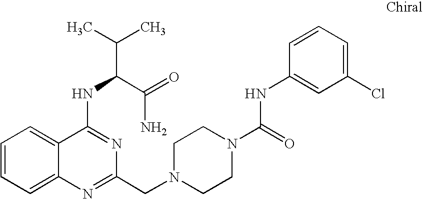 | 496.0 | 496.3 |
| 24-25 | 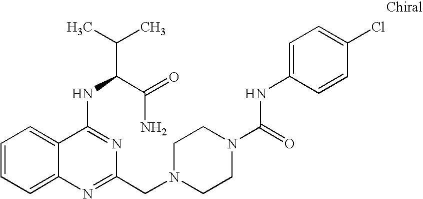 | 496.0 | 496.3 |
| 24-26 | 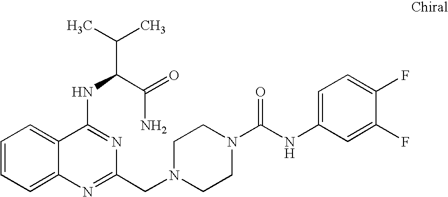 | 497.6 | 498.3 |
| 24-27 | 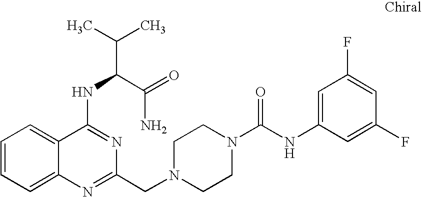 | 497.6 | 498.3 |
| 24-28 | 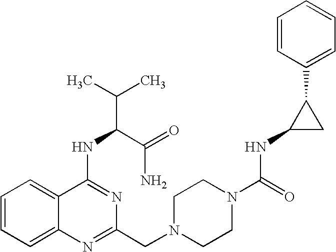 | 501.6 | 502.3 |
| 24-29 | 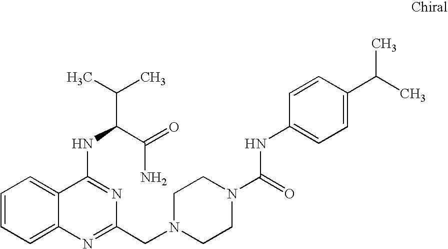 | 503.7 | 504.3 |
| 24-30 | 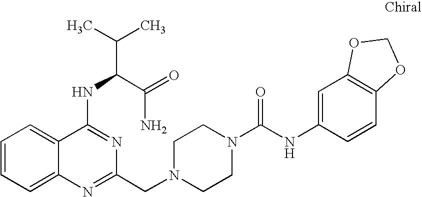 | 505.6 | 506.3 |
| 24-31 | 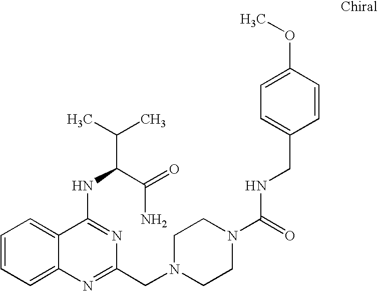 | 505.6 | 506.3 |
| 24-32 | 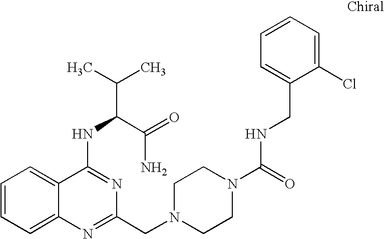 | 510.0 | 510.3 |
| 24-33 | 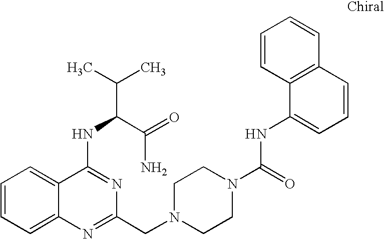 | 511.6 | 512.3 |
| 24-34 | 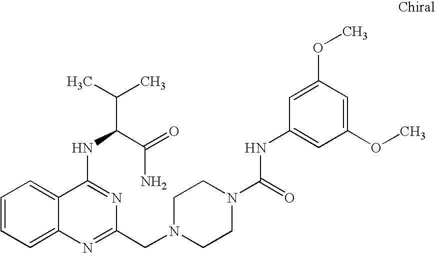 | 521.6 | 522.3 |
| 24-35 | 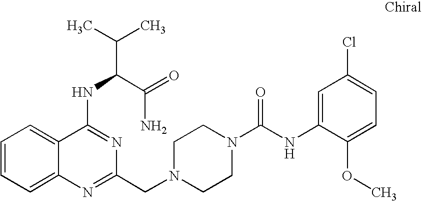 | 526.0 | 526.3 |
| 24-36 | 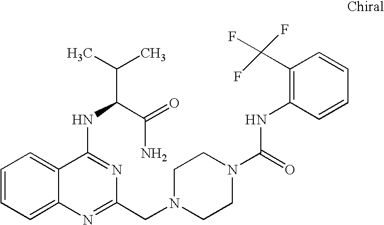 | 529.6 | 530.3 |
| 24-37 | 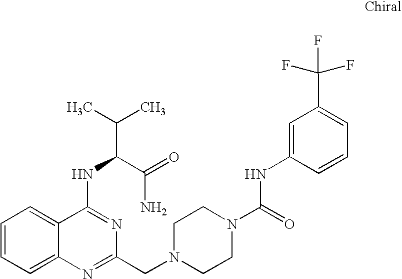 | 529.6 | 5303 |
| 24-38 | 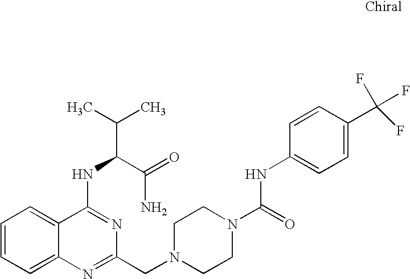 | 529.6 | 530.3 |
| 24-39 | 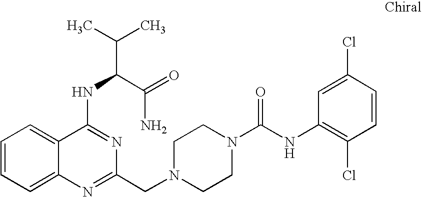 | 530.5 | 530.3 |
| 24-40 | 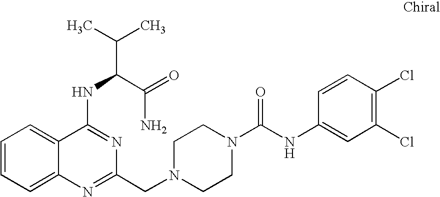 | 530.5 | 530.3 |
| 24-41 | 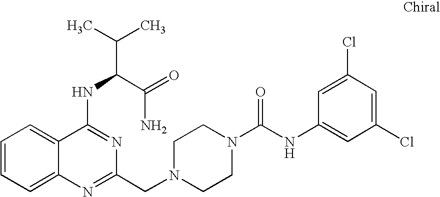 | 530.5 | 530.3 |
| 24-42 | 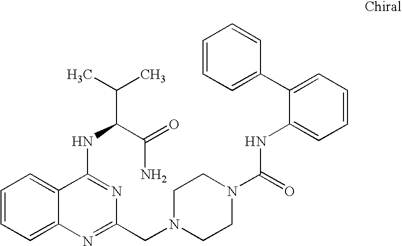 | 537.7 | 538.3 |
| 24-43 | 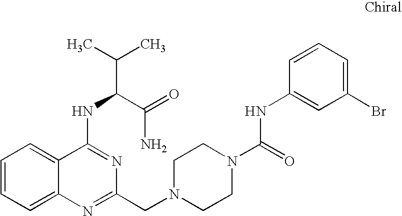 | 540.5 | 542.3 |
| 24-44 | 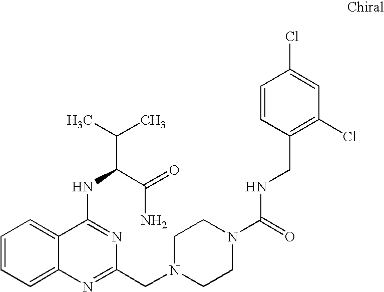 | 544.5 | 544.3 |
| 24-45 | 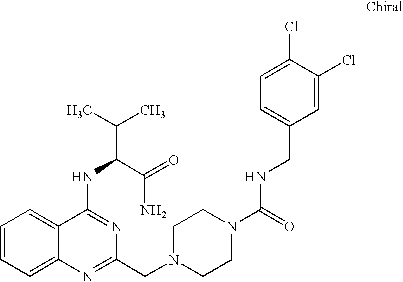 | 544.5 | 544.3 |
| 24-46 | 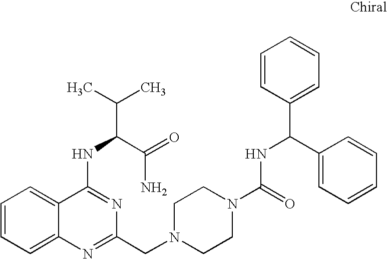 | 551.7 | 552.3 |
| 24-47 | 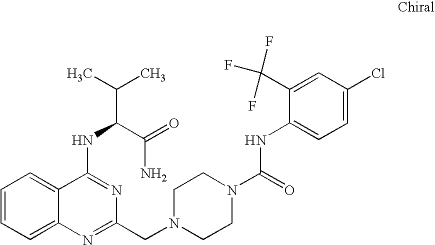 | 564.0 | 564.3 |
| 24-48 | 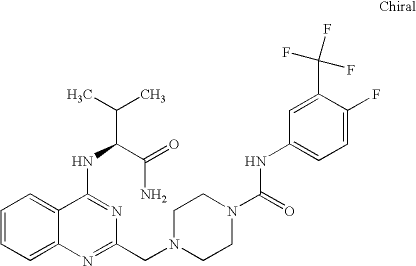 | 547.6 | 548.3 |
| 24-49 | 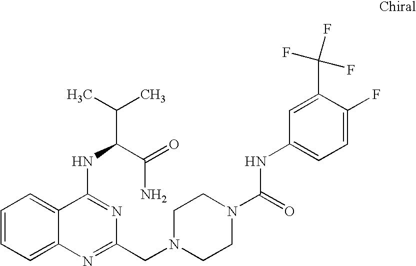 | 547.6 | 552.3 |

### Example 25

### Reaction of N,N-DIMETHYL-N′-[2-(PIPERAZIN-1-YLMETHYL)QUINAZOLIN-4-YL]PROPANE-1,3-DIAMINE with an ALDEHYDE/KETONE LIBRARY

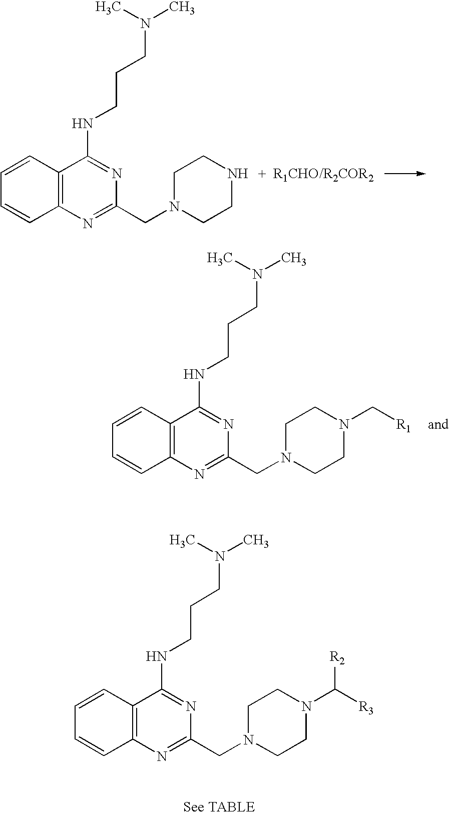

A stock solution of N,N-dimethyl-N′-[2-(piperazin-1-ylmethyl)quinazolin-4-yl]propane-1,3-diamine (1 mL, 0.0273 mmoles) (prepared as described in Preparative Example 24 above) in DCE was added to the tubes in four 24 tube Bohdan Miniblocks. A 1M stock solution of each of the individual aldehydes R₆CHO and ketones (R₂COR₃) in THF (0.1 mL, 0.0983 mmoles) was then added, one to each tube. Sodium triacetoxyborohydride (18 mg, 0.0846 mmoles) was added to each tube, followed by additional DCE (0.5 mL). The Miniblocks were sealed and shaken at 25° C. for 20 h. Methanol (0.5 mL) was added to each tube. MP-TsOH resin (˜0.12 g) was added to each tube and the blocks were shaken at 25° C. for 4 h. The tubes were drained and the resin was washed three times with methanol, shaking for 5 min each time, to remove unreacted reagents. Ammonia in methanol (2N, 2 mL) was added to each tube and the Miniblocks were again shaken at 25° C. for 20 min. The methanol filtrates were collected and the resin was again shaken with ammonia in methanol (2N, 2 mL). The combined filtrates from each tube, were evaporated to dryness overnight, on a Speedvac concentrator. The resulting samples were analyzed by LCMS and any samples that were <70% pure, were further purified by preparative LCMS. The compounds that were >70% pure are listed in the table below.

### Examples 25-1 Through 25-94

| | | | | | SPA Assay |
|---|---|---|---|---|---|
| | | | | PROFILE- | (% residual |
| | | | | RATION ASSAY | T @ 2 |
| | | | LCMS | EC50 MB468 | ug/mL of |
| EXAMPLE | STRUCTURE | MW | M+ | (uM) | drug) |
| 25-1 | 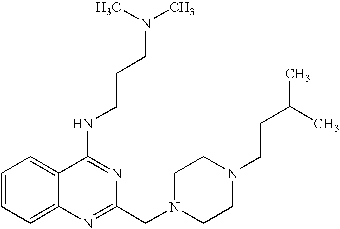 | 398.6 | 399.2 | | |
| 25-2 | 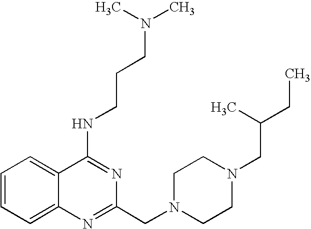 | 398.6 | 399.2 | | |
| 25-3 | 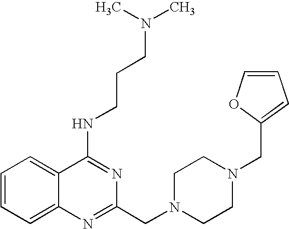 | 408.6 | 409.2 | | |
| 25-4 | 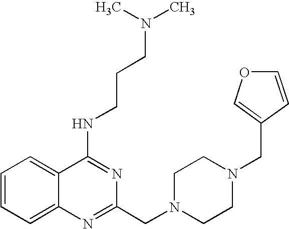 | 408.6 | 409.2 | | |
| 25-5 | 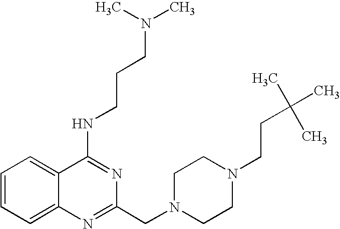 | 412.6 | 413.2 | | |
| 25-6 | 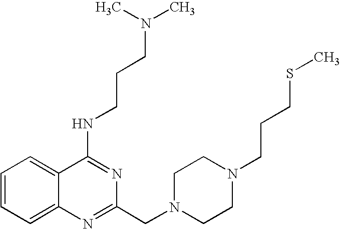 | 416.6 | 417.2 | | |
| 25-7 | 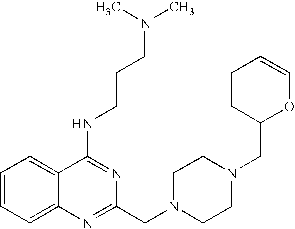 | 424.6 | 425.2 | | |
| 25-8 | 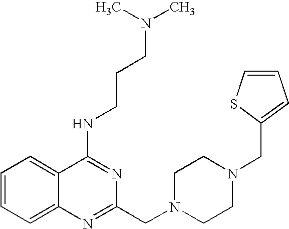 | 424.6 | 425.2 | | |
| 25-9 | 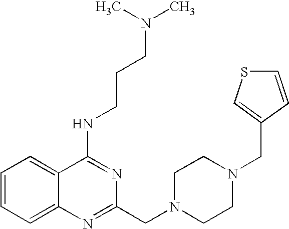 | 424.6 | 425.2 | | |
| 25-10 | 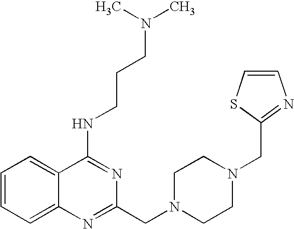 | 425.6 | 426.2 | | |
| 25-11 | 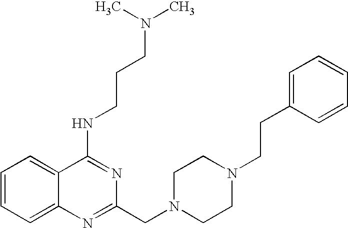 | 432.6 | 433.2 | | |
| 25-12 | 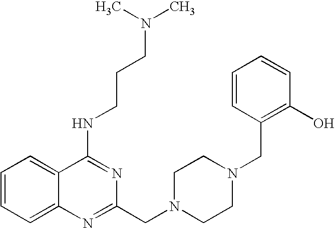 | 434.6 | 435.2 | | |
| 25-13 | 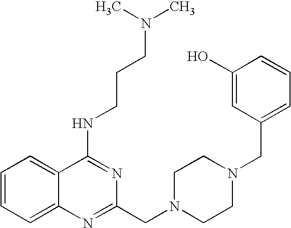 | 434.6 | 435.2 | | |
| 25-14 | 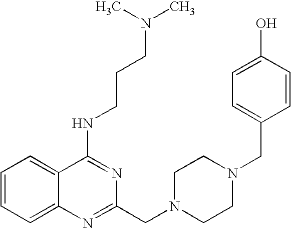 | 434.6 | 435.2 | | |
| 25-15 | 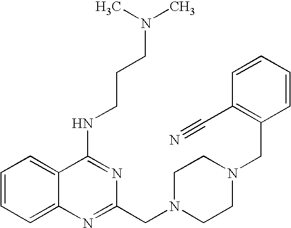 | 443.6 | 444.2 | | |
| 25-16 | 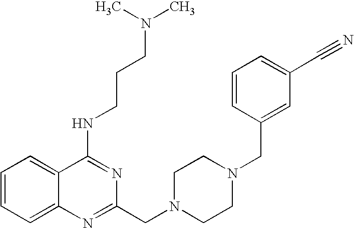 | 443.6 | 444.2 | | |
| 25-17 | 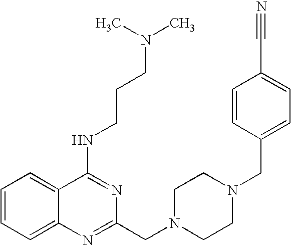 | 443.6 | 444.2 | | |
| 25-18 | 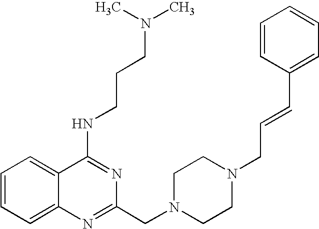 | 444.6 | 445.2 | | |
| 25-19 | 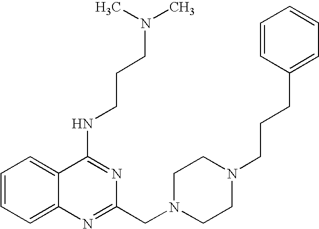 | 446.6 | 447.2 | | |
| 25-20 | 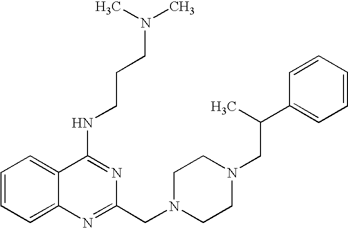 | 446.6 | 447.2 | | |
| 25-21 | 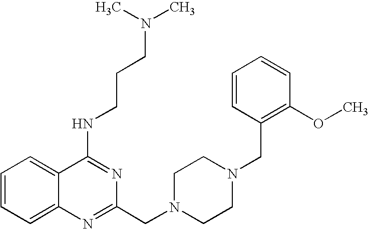 | 448.6 | 449.2 | | |
| 25-22 | 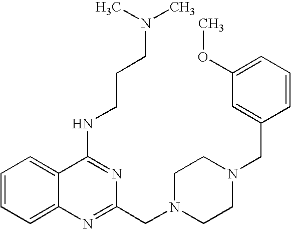 | 448.6 | 449.2 | | |
| 25-23 | 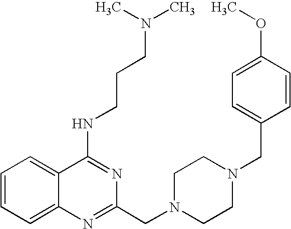 | 448.6 | 449.2 | | |
| 25-24 | 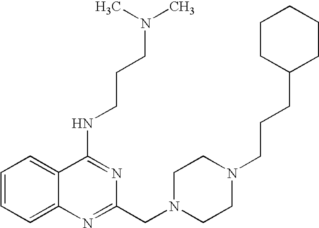 | 452.7 | 453.2 | | |
| 25-25 | 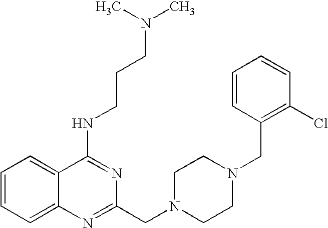 | 453.0 | 453.2 | | |
| 25-26 | 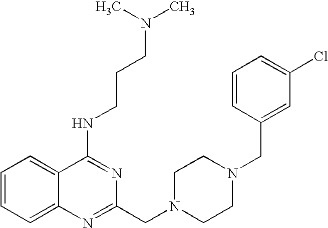 | 453.0 | 453.2 | | |
| 25-27 | 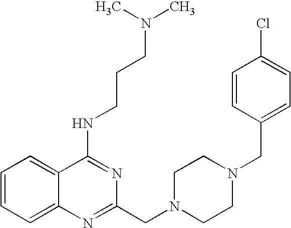 | 453.0 | 453.2 | | |
| 25-28 | 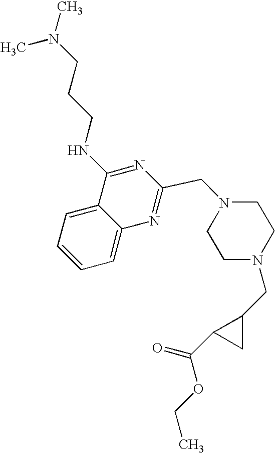 | 454.6 | 455.3 | | |
| 25-29 | 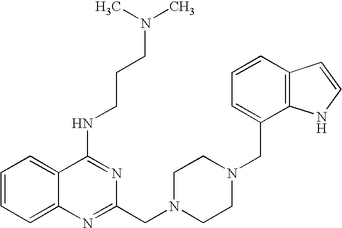 | 457.6 | 458.3 | C | B |
| 25-30 | 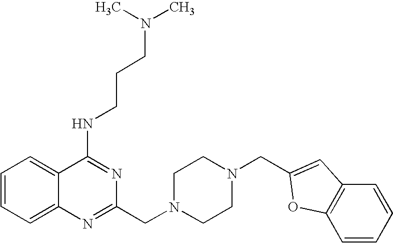 | 458.6 | 459.3 | | |
| 25-31 | 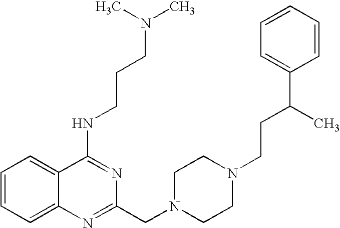 | 460.7 | 461.3 | | |
| 25-32 | 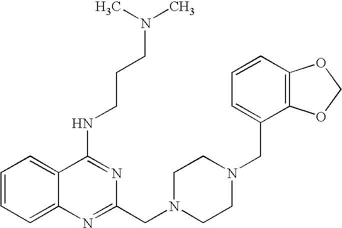 | 462.6 | 463.3 | | |
| 25-33 | 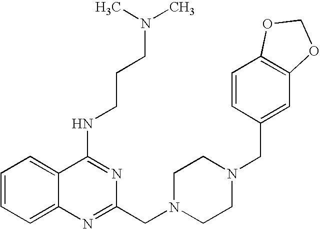 | 462.6 | 463.3 | | |
| 25-34 | 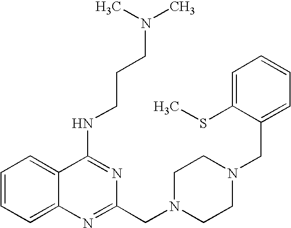 | 464.7 | 465.3 | | |
| 25-35 | 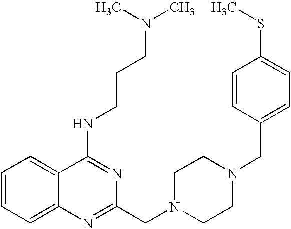 | 464.7 | 465.3 | | |
| 25-36 | 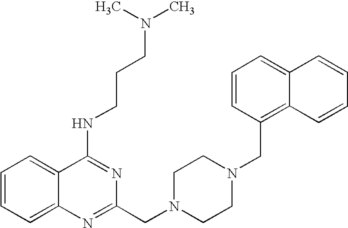 | 468.6 | 469.3 | C | B |
| 25-37 | 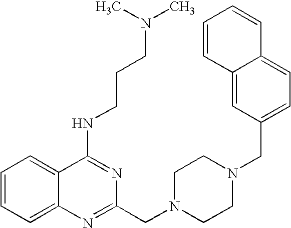 | 468.6 | 469.3 | | |
| 25-38 | 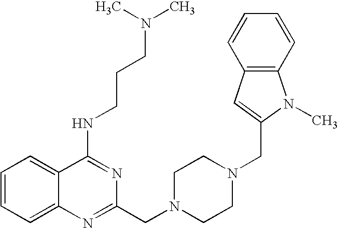 | 471.7 | 472.3 | | |
| 25-39 | 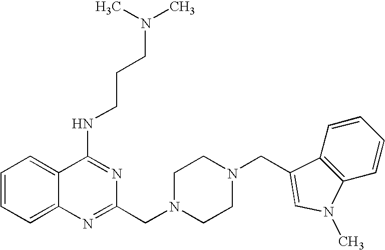 | 471.7 | 472.3 | | |
| 25-40 | 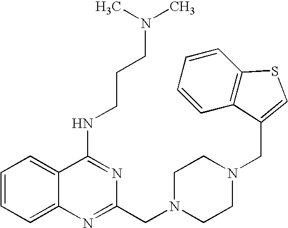 | 474.7 | 475.3 | | |
| 25-41 | 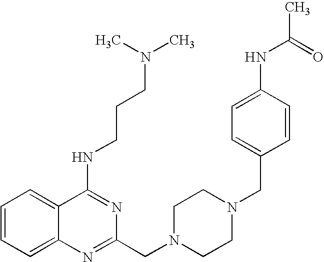 | 475.6 | 476.3 | | |
| 25-42 | 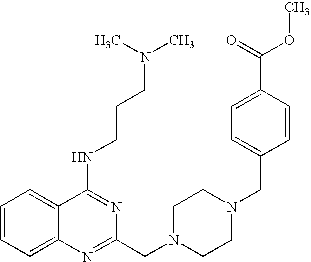 | 476.6 | 477.3 | | |
| 25-43 | 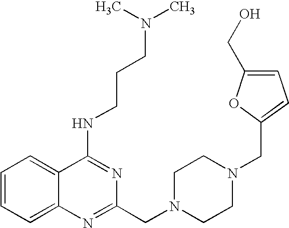 | 438.6 | 439.2 | | |
| 25-44 | 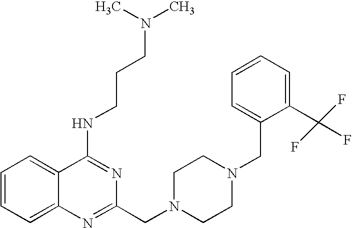 | 486.6 | 487.3 | | |
| 25-45 | 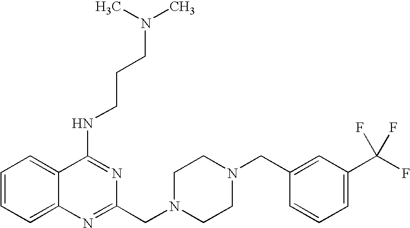 | 486.6 | 487.3 | | |
| 25-46 | 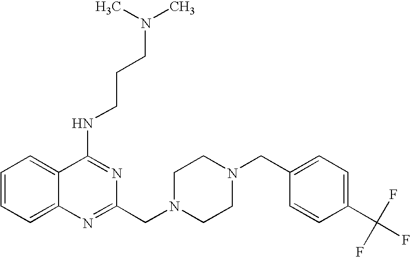 | 486.6 | 487.3 | | |
| 25-47 | 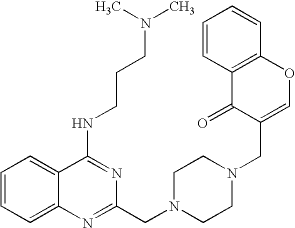 | 486.6 | 487.3 | | |
| 25-48 | 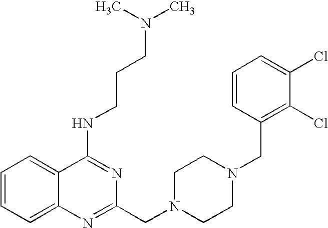 | 487.5 | 487.3 | | |
| 25-49 | 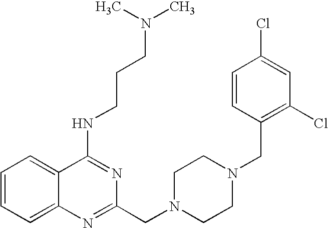 | 487.5 | 487.3 | | |
| 25-50 | 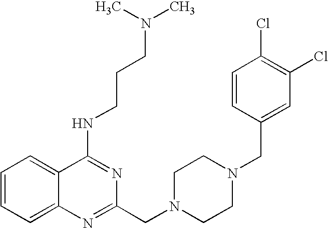 | 487.5 | 487.3 | | |
| 25-51 | 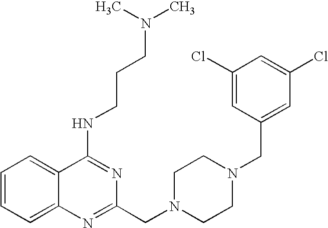 | 487.5 | 487.3 | | |
| 25-52 | 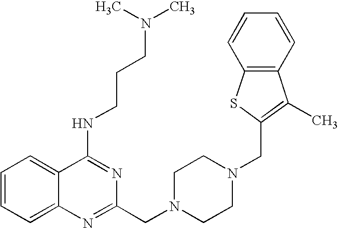 | 488.7 | 489.3 | | |
| 25-53 | 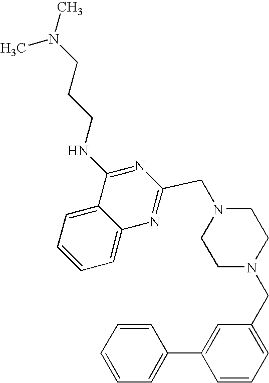 | 494.7 | 495.3 | | |
| 25-54 | 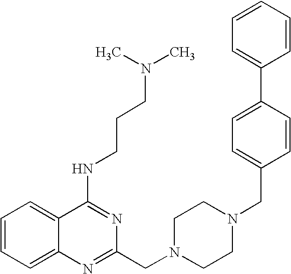 | 494.7 | 495.3 | | |
| 25-55 | 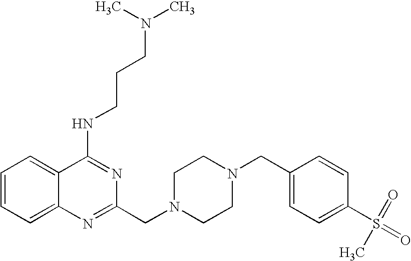 | 496.7 | 497.3 | | |
| 25-56 | 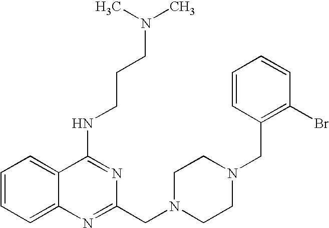 | 497.5 | 497.3 | | |
| 25-57 | 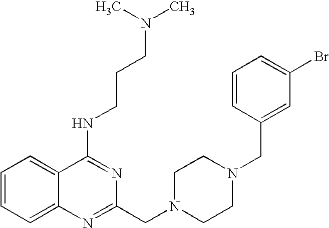 | 497.5 | 497.3 | | |
| 25-58 | 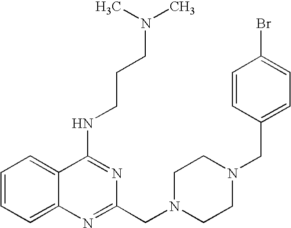 | 497.5 | 497.3 | | |
| 25-59 | 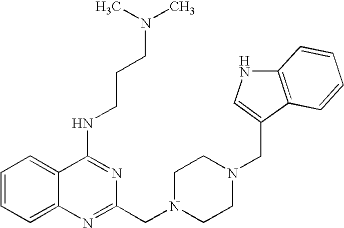 | 457.6 | 458.3 | | |
| 25-60 | 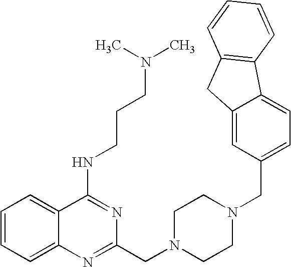 | 506.7 | 507.3 | | |
| 25-61 | 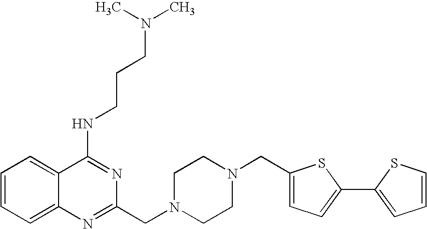 | 506.7 | 507.3 | | |
| 25-62 | 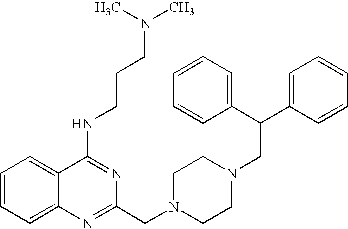 | 508.7 | 509.3 | | |
| 25-63 | 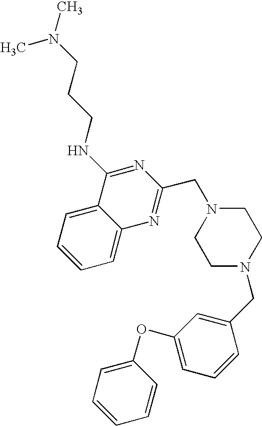 | 510.7 | 511.3 | | |
| 25-64 | 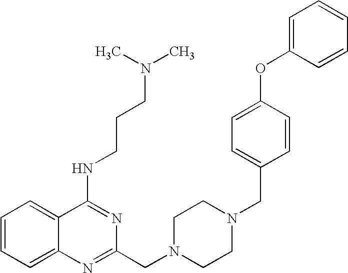 | 510.7 | 511.3 | | |
| 25-65 | 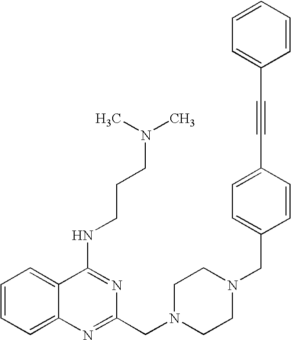 | 518.7 | 519.3 | | |
| 25-66 | 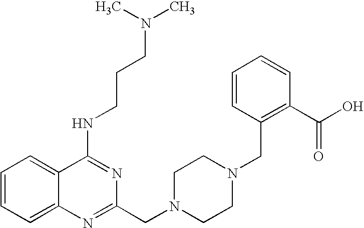 | 462.6 | 463.3 | | |
| 25-67 | 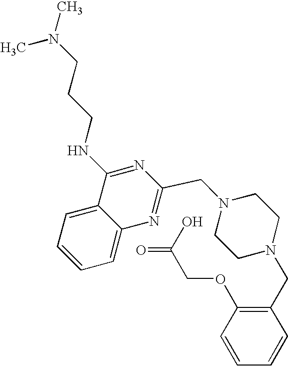 | 492.6 | 493.3 | | |
| 25-68 | 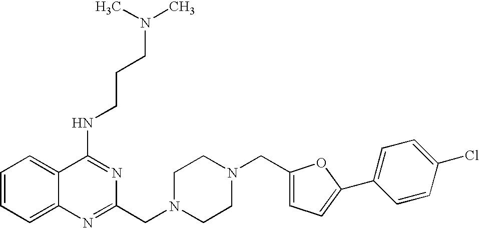 | 519.1 | 519.3 | | |
| 25-69 | 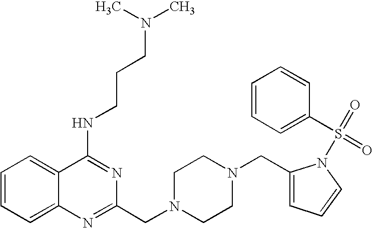 | 547.7 | 548.3 | | |
| 25-70 | 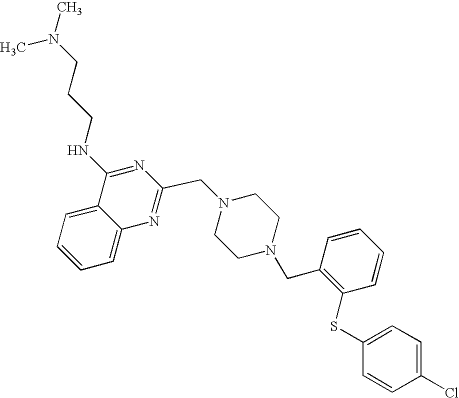 | 561.2 | 561.3 | | |
| 25-71 | 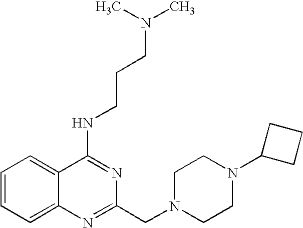 | 382.6 | 383.2 | | |
| 25-72 | 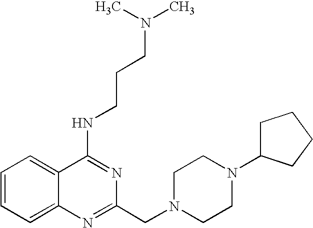 | 396.6 | 397.2 | | |
| 25-73 | 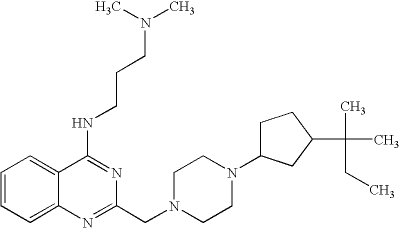 | 466.7 | 467.3 | C | C |
| 25-74 | 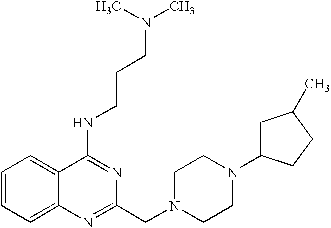 | 410.6 | 411.2 | | |
| 25-75 | 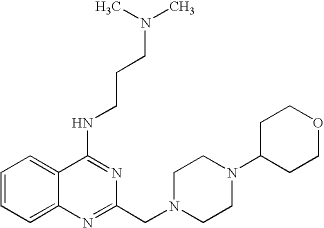 | 412.6 | 411.2 | | |
| 25-76 | 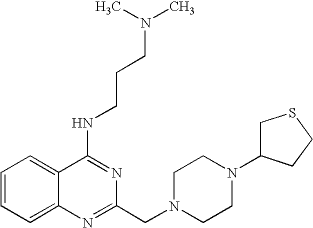 | 414.6 | 415.2 | | |
| 25-77 | 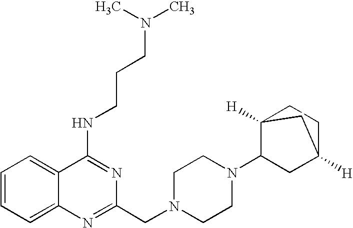 | 422.6 | 423.2 | | |
| 25-78 | 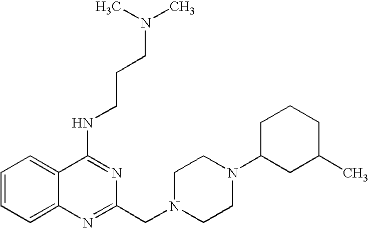 | 424.6 | 425.2 | | |
| 25-79 | 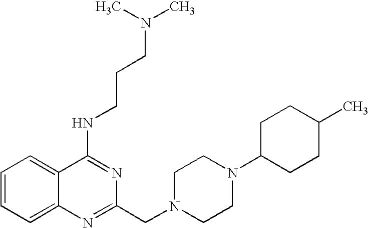 | 424.6 | 425.2 | | |
| 25-80 | 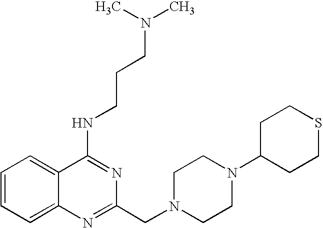 | 428.6 | 429.2 | | |
| 25-81 | 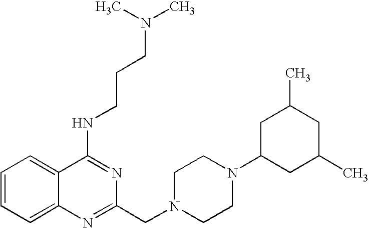 | 438.7 | 439.2 | | |
| 25-82 | 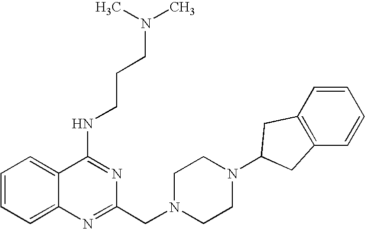 | 444.6 | 445.2 | | |
| 25-83 | 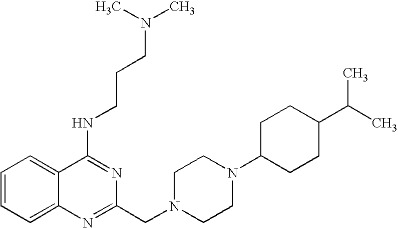 | 452.7 | 453.2 | | |
| 25-84 | 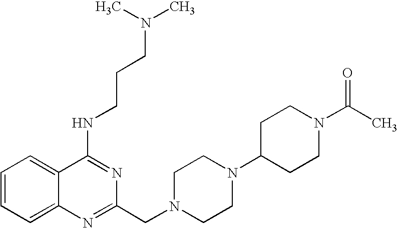 | 453.6 | 454.2 | | |
| 25-85 | 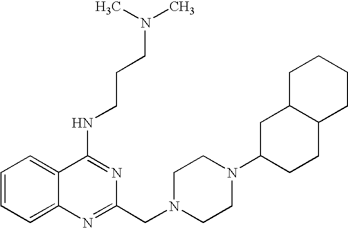 | 464.7 | 465.3 | | |
| 25-86 | 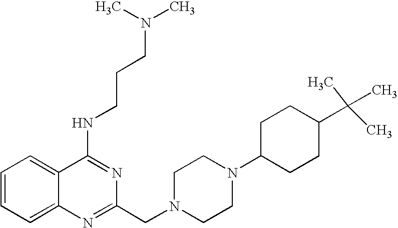 | 466.7 | 467.3 | | |
| 25-87 | 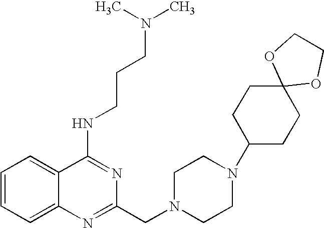 | 468.6 | 469.3 | | |
| 25-88 | 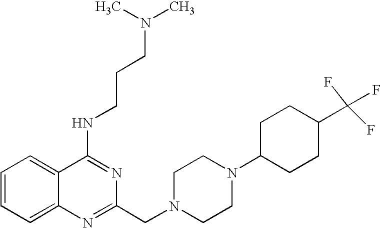 | 478.6 | 479.7 | | |
| 25-89 | 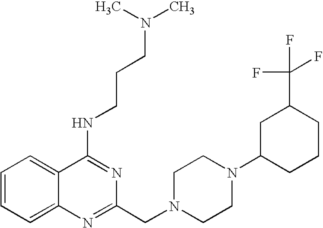 | 478.6 | 479.3 | | |
| 25-90 | 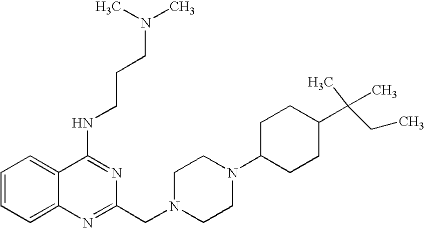 | 480.7 | 481.3 | B | B |
| 25-91 | 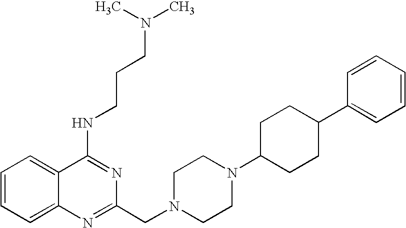 | 486.7 | 487.3 | | |
| 25-92 | 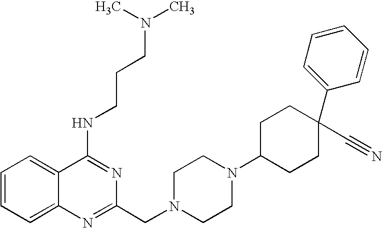 | 511.7 | 512.3 | | |
| 25-93 | 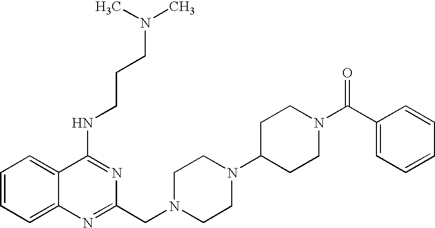 | 515.7 | 516.3 | | |
| 25-94 | 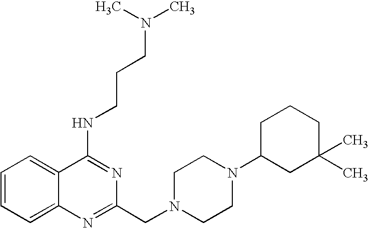 | 438.7 | 439.2 | | |

### Example 26

### Reaction of N,N-DIMETHYL-N′-[2-(PIPERAZIN-1-YLMETHYL)QUINAZOLIN-4-YL]PROPANE-1,3-DIAMINE with an ACID LIBRARY

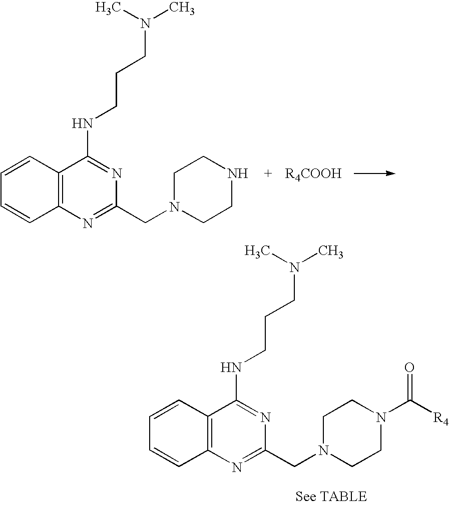

A stock solution of N,N-dimethyl-N′-[2-(piperazin-1-ylmethyl)quinazolin-4-yl]propane-1,3-diamine (1 mL, 0.0233 mmoles) (prepared as described in Preparative Example 24 above) in DCE was added to the tubes in three 24 tube Bohdan Miniblocks. PS-EDC resin (41 mg, 0.0583 mmoles) was added to each tube, followed by a stock solution of HOBT in THF (0.5 mL, 0.0350 mmoles). 1M stock solutions of each of the individual acids (R₄COOH) (0.03 mL, 0.0303 mmoles) were added to the tubes and the Miniblocks were sealed and shaken at 25° C. for 21 h. PS-Isocyanate resin (3 equivalents, 0.0699 mmoles) was added to each tube, followed by PS-Trisamine resin (6 equivalents, 0.1398 mmoles) and the Miniblocks were shaken at 25° C. for 4 h. The tubes were drained and the resin was washed with THF (2×1 mL), shaking for 5 min each time. The filtrates from each tube were combined and evaporated to dryness overnight on a Speedvac concentrator. The resulting samples were evaluated by LCMS and those that were >70% pure are listed in the table below.

### Examples 26-1 Through 26-70

| EXAMPLE | STRUCTURE | MW | LCMS m/z |
|---|---|---|---|
| 26-1 | 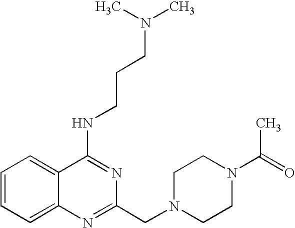 | 370.5 | 371.1 |
| 26-2 | 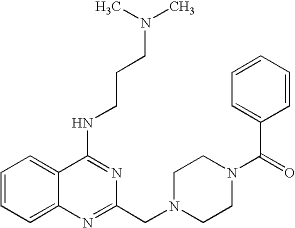 | 432.6 | 433.1 |
| 26-3 | 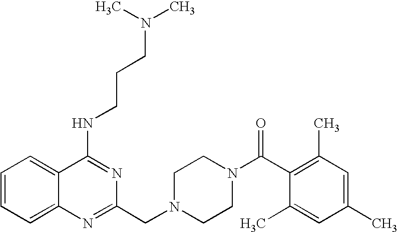 | 474.7 | 475.1 |
| 26-4 | 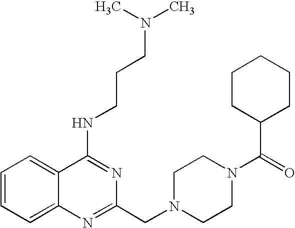 | 438.6 | 439.1 |
| 26-5 | 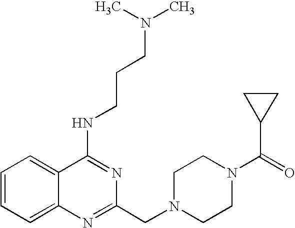 | 396.5 | 397.3 |
| 26-6 | 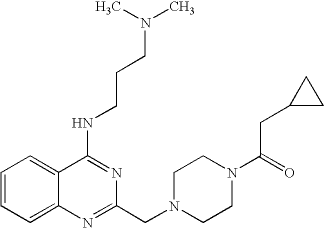 | 410.6 | 411.2 |
| 26-7 | 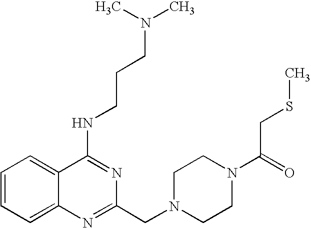 | 416.6 | 417.2 |
| 26-8 | 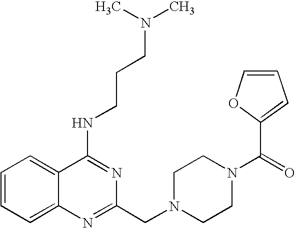 | 422.5 | 423.2 |
| 26-9 | 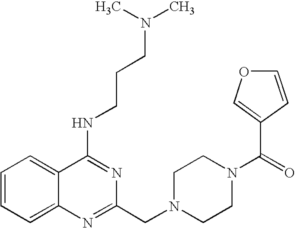 | 422.5 | 423.2 |
| 26-10 | 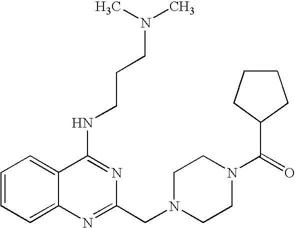 | 424.6 | 425.2 |
| 26-11 | 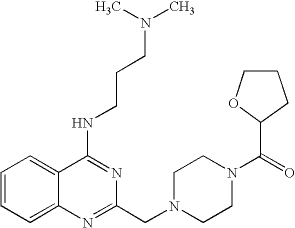 | 426.6 | 427.2 |
| 26-12 | 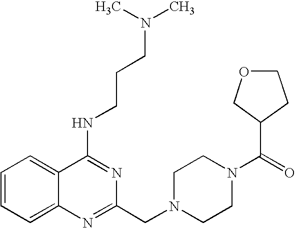 | 426.6 | 427.2 |
| 26-13 | 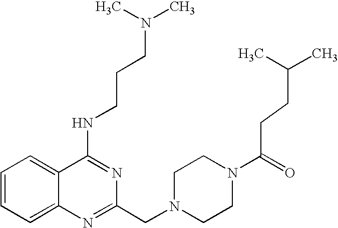 | 426.6 | 427.2 |
| 26-14 | 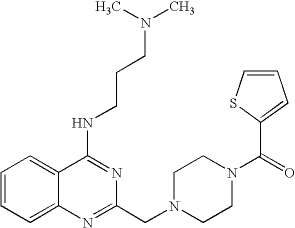 | 438.6 | 439.2 |
| 26-15 | 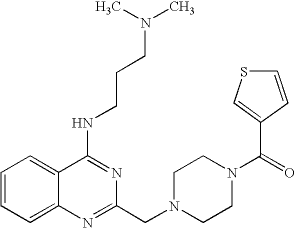 | 438.6 | 439.2 |
| 26-16 | 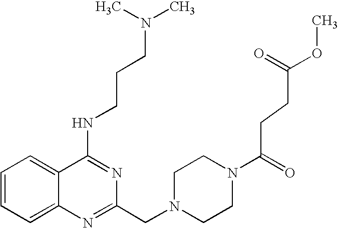 | 442.6 | 443.2 |
| 26-17 | 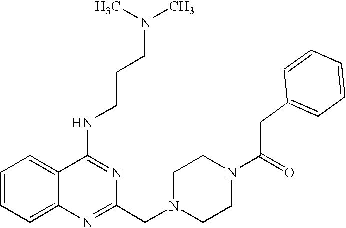 | 446.6 | 447.2 |
| 26-18 | 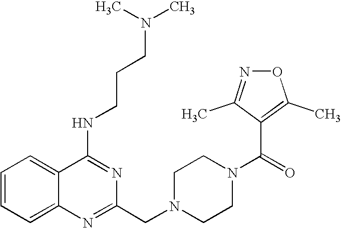 | 451.6 | 452.2 |
| 26-19 | 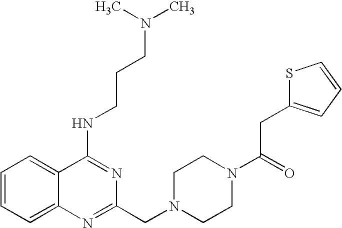 | 452.6 | 453.2 |
| 26-20 | 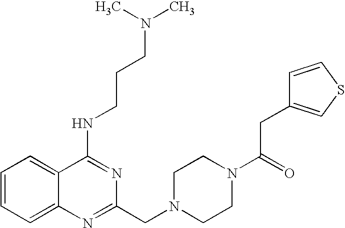 | 452.6 | 453.2 |
| 26-21 | 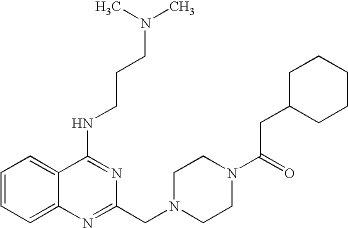 | 452.6 | 453.2 |
| 26-22 | 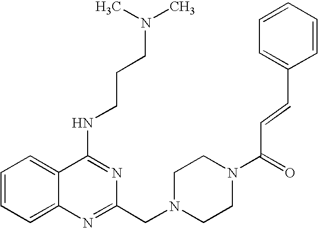 | 458.6 | 459.3 |
| 26-23 | 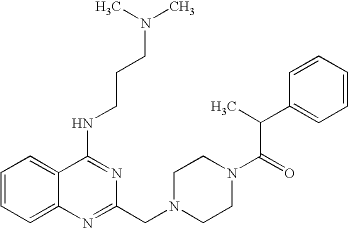 | 460.6 | 461.3 |
| 26-24 | 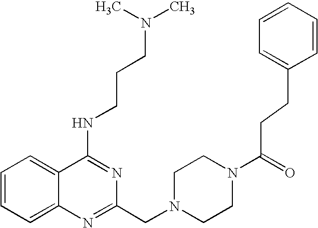 | 460.6 | 461.3 |
| 26-25 | 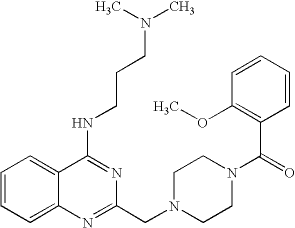 | 462.6 | 463.3 |
| 26-26 | 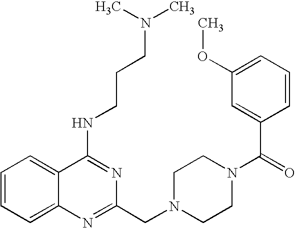 | 462.6 | 463.3 |
| 26-27 | 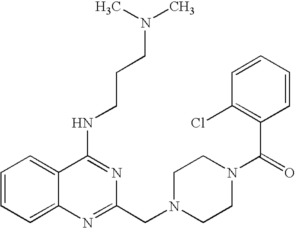 | 467.0 | 467.3 |
| 26-28 | 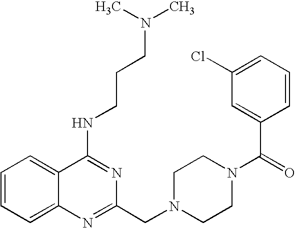 | 467.0 | 467.3 |
| 26-29 | 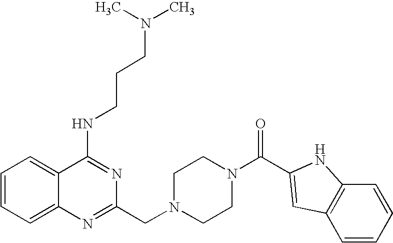 | 471.6 | 472.3 |
| 26-30 | 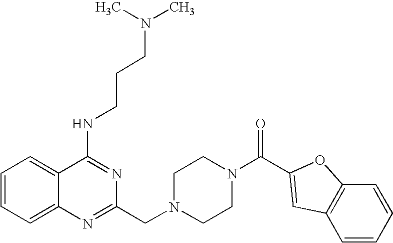 | 472.6 | 473.3 |
| 26-31 | 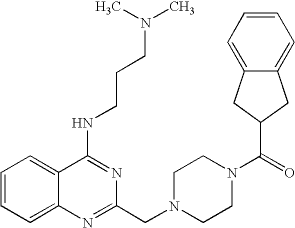 | 472.6 | 473.3 |
| 26-32 | 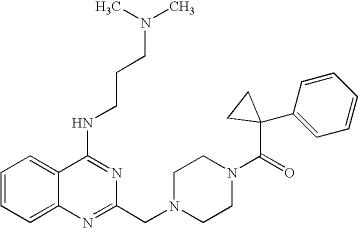 | 472.6 | 473.3 |
| 26-33 | 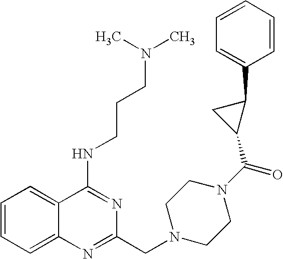 | 472.6 | 473.3 |
| 26-34 | 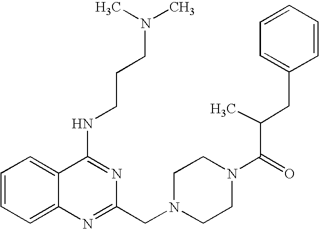 | 474.7 | 475.3 |
| 26-35 | 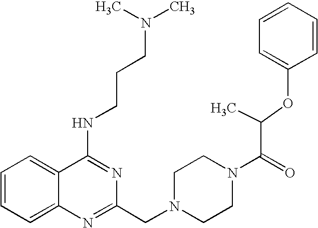 | 476.6 | 477.3 |
| 26-36 | 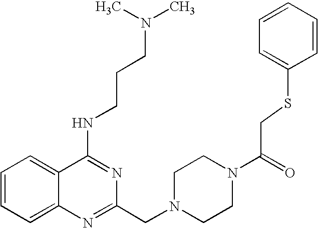 | 478.7 | 479.3 |
| 26-37 | 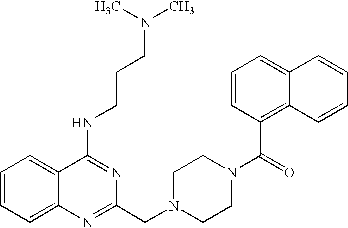 | 482.6 | 483.3 |
| 26-38 | 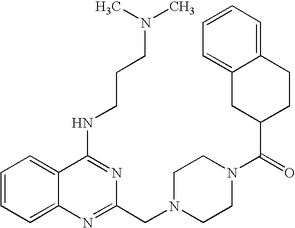 | 486.7 | 487.3 |
| 26-39 | 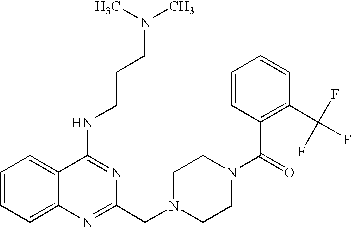 | 500.6 | 501.3 |
| 26-40 | 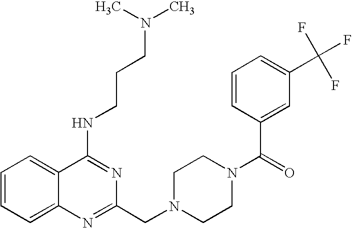 | 500.6 | 501.3 |
| 26-41 | 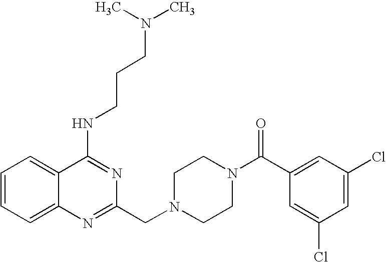 | 501.5 | 503.3 |
| 26-42 | 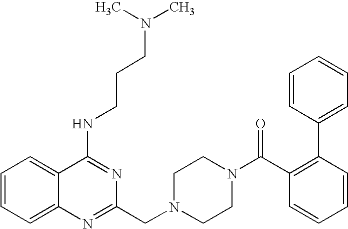 | 508.7 | 509.3 |
| 26-43 | 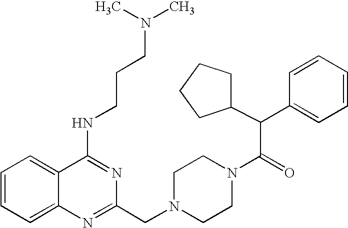 | 514.7 | 515.3 |
| 26-44 | 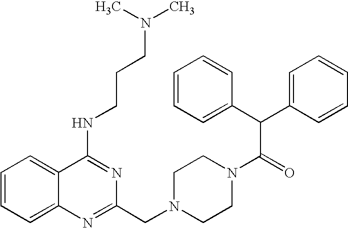 | 522.7 | 523.3 |
| 26-45 | 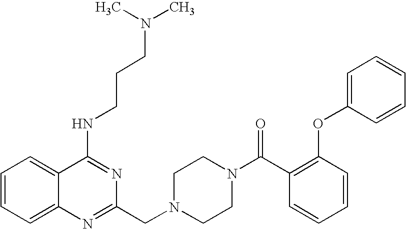 | 524.7 | 525.3 |
| 26-46 | 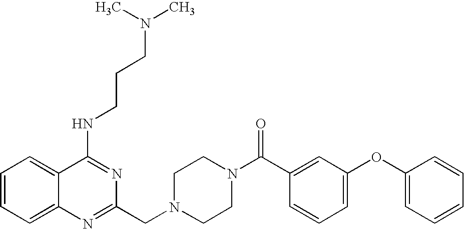 | 524.7 | 525.3 |
| 26-47 | 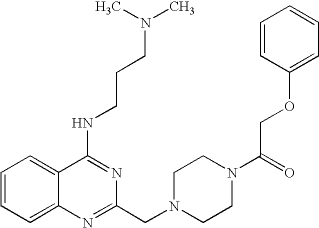 | 462.6 | 463.3 |
| 26-48 | 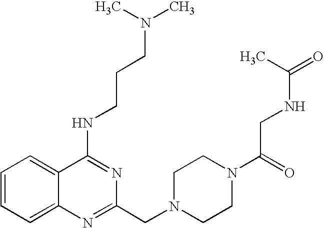 | 427.6 | 428.2 |
| 26-49 | 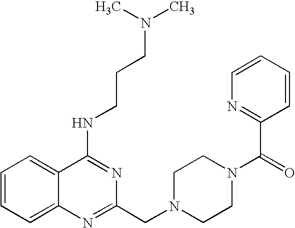 | 433.6 | 434.2 |
| 26-50 | 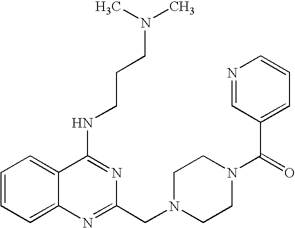 | 433.6 | 434.2 |
| 26-51 | 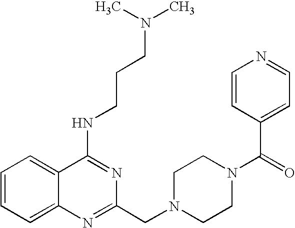 | 433.6 | 434.2 |
| 26-52 | 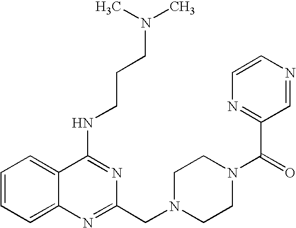 | 434.5 | 435.2 |
| 26-53 | 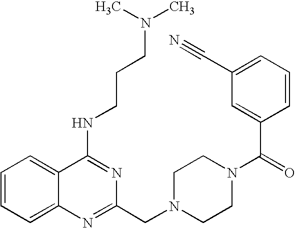 | 457.6 | 458.3 |
| 26-54 | 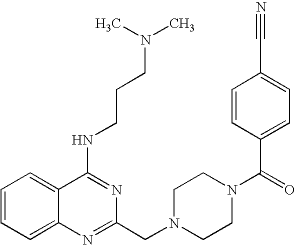 | 457.6 | 458.3 |
| 26-55 | 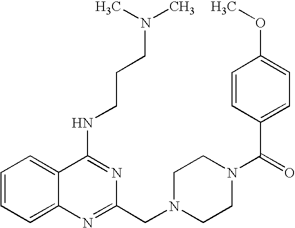 | 462.6 | 463.3 |
| 26-56 | 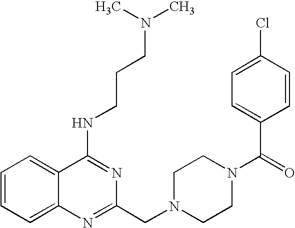 | 467.0 | 467.3 |
| 26-57 | 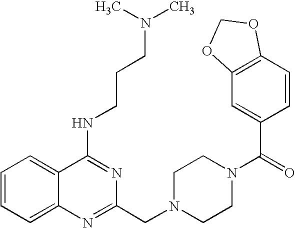 | 476.6 | 477.3 |
| 26-58 | 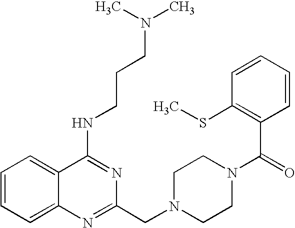 | 478.7 | 479.3 |
| 26-59 | 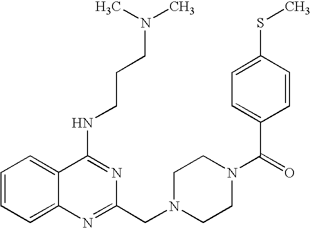 | 478.7 | 479.3 |
| 26-60 | 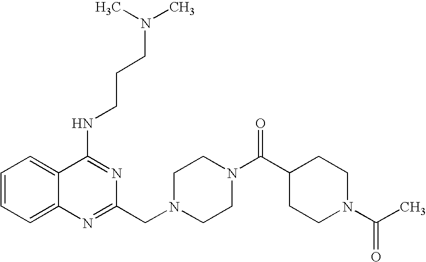 | 481.6 | 482.3 |
| 26-61 | 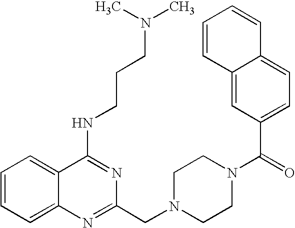 | 482.6 | 483.3 |
| 26-62 | 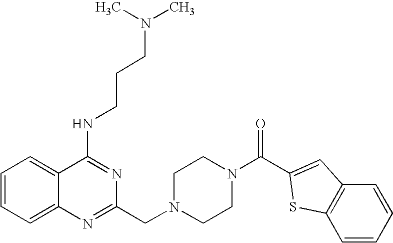 | 488.7 | 489.3 |
| 26-63 | 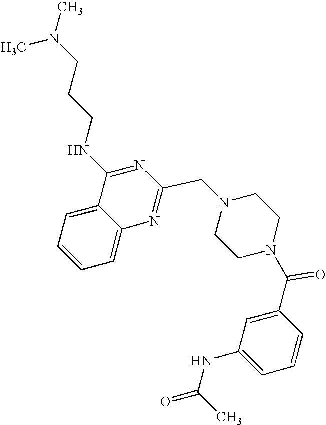 | 489.6 | 490.3 |
| 26-64 | 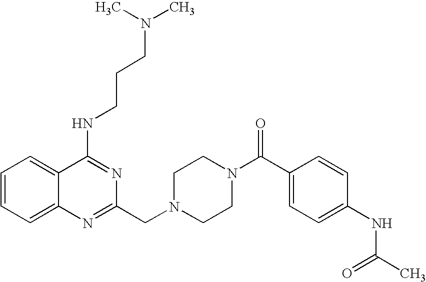 | 489.6 | 490.3 |
| 26-65 | 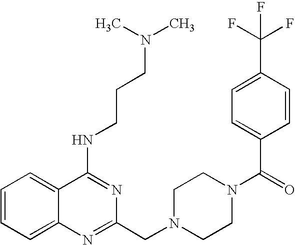 | 500.6 | 501.3 |
| 26-66 | 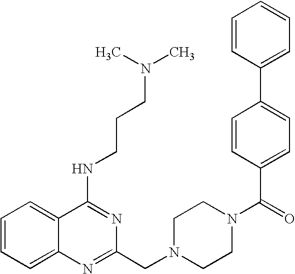 | 508.7 | 509.3 |
| 26-67 | 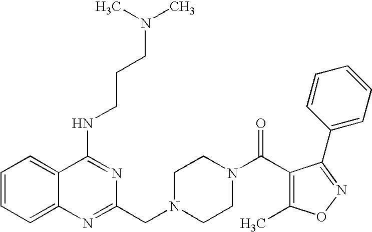 | 513.6 | 514.3 |
| 26-68 | 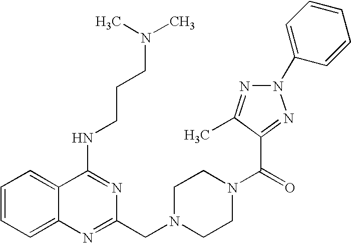 | 513.7 | 514.3 |
| 26-69 | 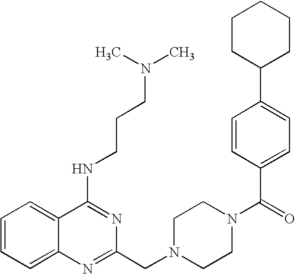 | 514.7 | 515.3 |
| 26-70 | 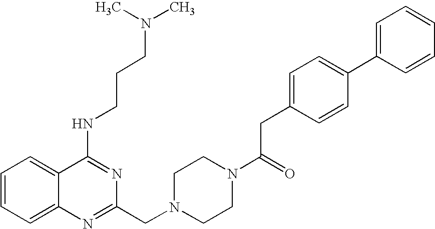 | 522.7 | 523.3 |

### Example 27

### Reaction of N,N-DIMETHYL-N′-[2-(PIPERAZIN-1-YLMETHYL)QUINAZOLIN-4-YL]PROPANE-1,3-DIAMINE with a SULFONYL CHLORIDE LIBRARY

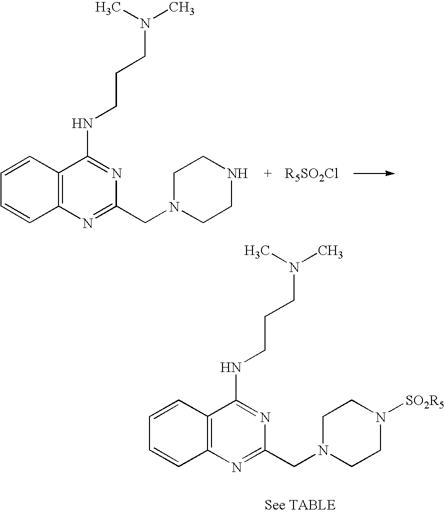

A stock solution of N,N-dimethyl-N′-[2-(piperazin-1-ylmethyl)quinazolin-4-yl]propane-1,3-diamine (1 mL, 0.0233 mmoles) (prepared as described in Preparative Example 24 above) in acetonitrile was added to the tubes in four 24 tube Bohdan Miniblocks. PS-DIEA resin (x mg, 0.0699 mmoles) was added to each of the tubes, followed by a 1M solution of each of the sulfonyl chlorides (R₅SO₂Cl) (1.5 equivalents, 0.0350 mmoles) in CH₃CN. Additional acetonitrile (0.5 mL) was added to each tube and the Miniblocks were sealed and shaken at 25° C. for 22 h. PS-Isocyanate resin (3 equivalents, 0.0699 mmoles) was added to each tube, followed by PS-Trisamine resin (6 equivalents, 0.14 mmoles) and the Miniblocks were shaken at 25° C. for 4 h. The tubes were drained and the resin was washed with acetonitrile (2×1 mL), shaking for 5 min each time. The filtrates from each tube were combined and evaporated to dryness overnight, on a Speedvac concentrator. The resulting samples were evaluated by LCMS and those that were >70% pure are listed in the table below.

### Examples 27-1 Through 27-75

| EXAMPLE | STRUCTURE | MW | LCMS m/z |
|---|---|---|---|
| 27-1 | 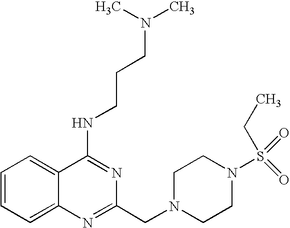 | 420.6 | 421.3 |
| 27-2 | 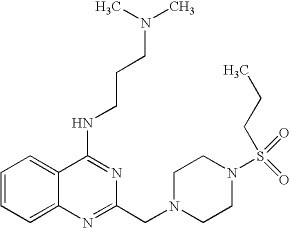 | 434.6 | 435.2 |
| 27-3 | 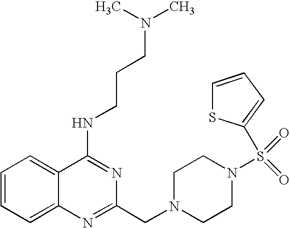 | 474.7 | 475.3 |
| 27-4 | 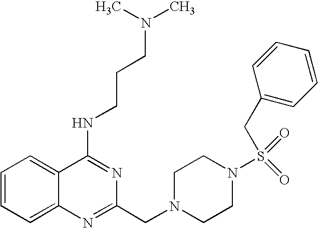 | 482.7 | 483.3 |
| 27-5 | 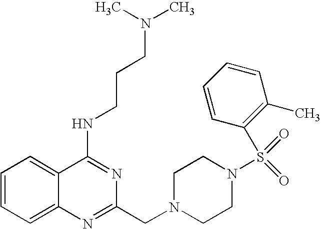 | 482.7 | 483.3 |
| 27-6 | 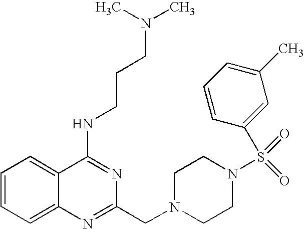 | 482.7 | 483.3 |
| 27-7 | 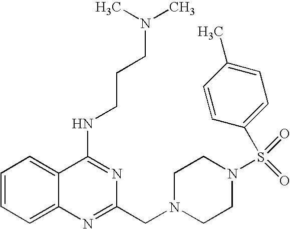 | 482.7 | 483.3 |
| 27-8 | 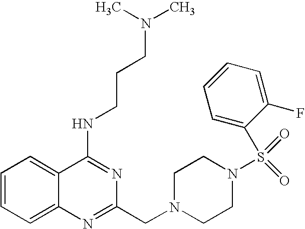 | 486.6 | 487.3 |
| 27-9 | 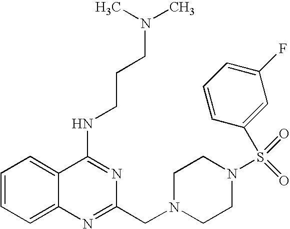 | 486.6 | 487.3 |
| 27-10 | 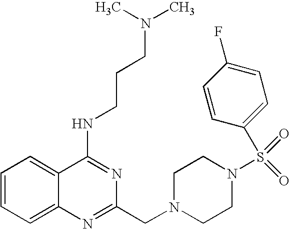 | 486.6 | 487.3 |
| 27-11 | 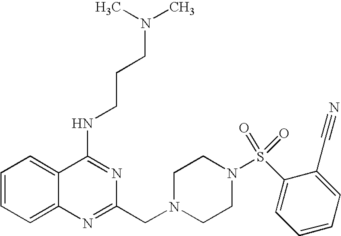 | 493.6 | 494.3 |
| 27-12 | 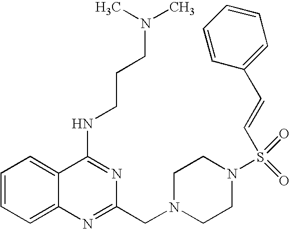 | 494.7 | 495.3 |
| 27-13 | 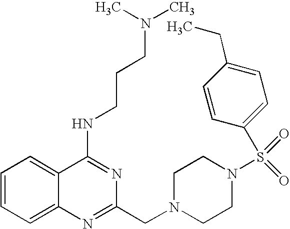 | 496.7 | 497.3 |
| 27-14 | 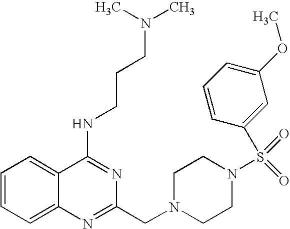 | 498.7 | 499.3 |
| 27-15 | 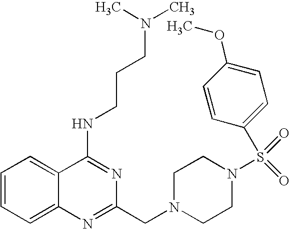 | 498.7 | 499.3 |
| 27-16 | 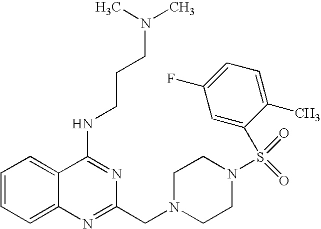 | 500.6 | 501.3 |
| 27-17 | 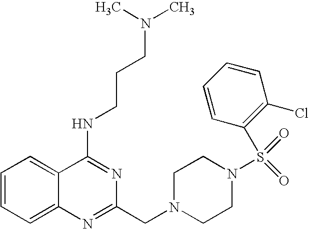 | 503.1 | 503.3 |
| 27-18 | 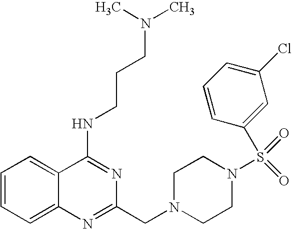 | 503.1 | 503.3 |
| 27-19 | 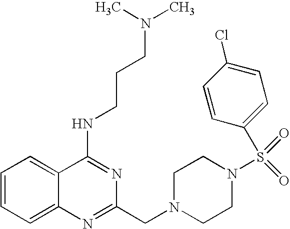 | 503.1 | 503.3 |
| 27-20 | 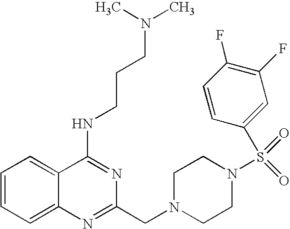 | 504.6 | 505.3 |
| 27-21 | 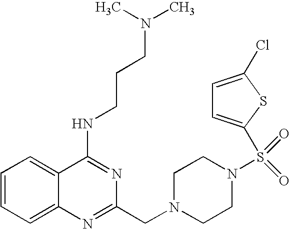 | 509.1 | 509.3 |
| 27-22 | 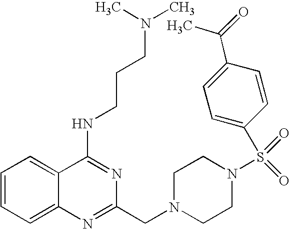 | 510.7 | 511.3 |
| 27-23 | 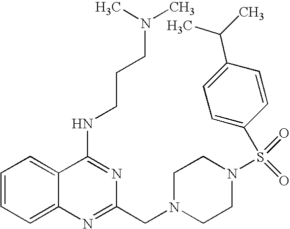 | 510.7 | 511.3 |
| 27-24 | 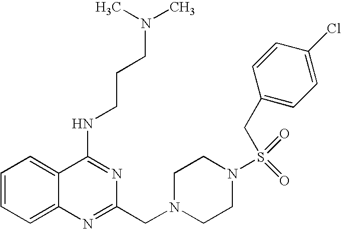 | 517.1 | 517.3 |
| 27-25 | 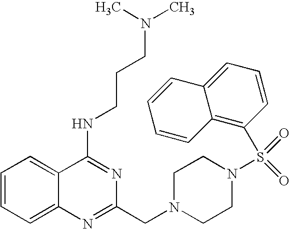 | 518.7 | 519.3 |
| 27-26 | 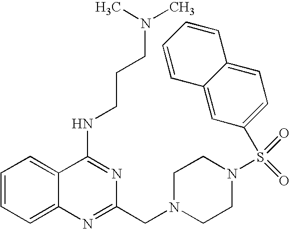 | 518.7 | 519.3 |
| 27-27 | 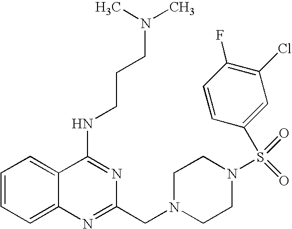 | 521.1 | 521.3 |
| 27-28 | 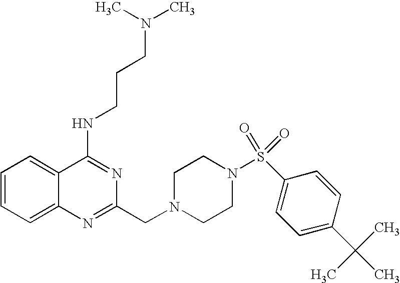 | 524.7 | 525.3 |
| 27-29 | 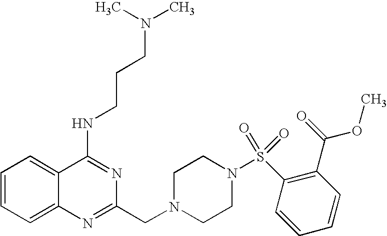 | 526.7 | 527.3 |
| 27-30 | 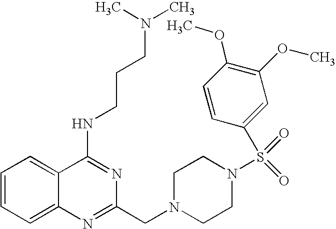 | 528.7 | 529.3 |
| 27-31 | 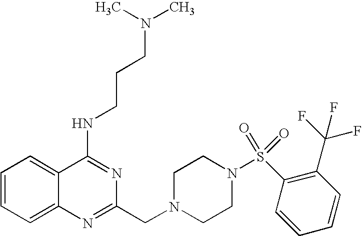 | 536.6 | 537.3 |
| 27-32 | 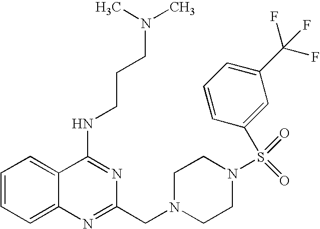 | 536.6 | 537.3 |
| 27-33 | 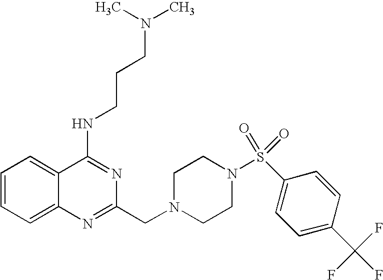 | 536.6 | 537.3 |
| 27-34 | 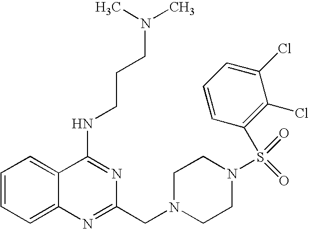 | 537.5 | 537.3 |
| 27-35 | 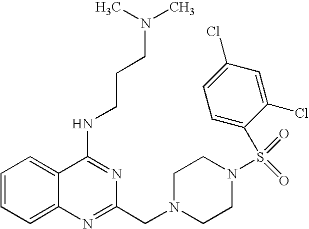 | 537.5 | 537.3 |
| 27-36 | 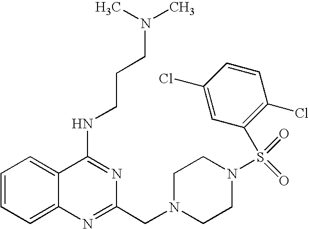 | 537.5 | 537.3 |
| 27-37 | 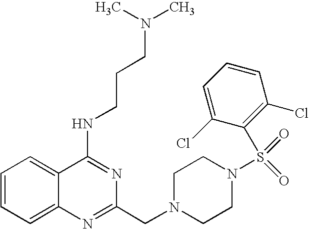 | 537.5 | 537.3 |
| 27-38 | 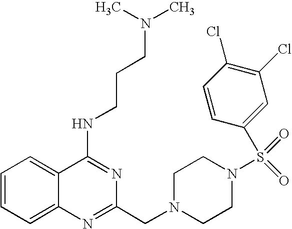 | 537.5 | 537.3 |
| 27-39 | 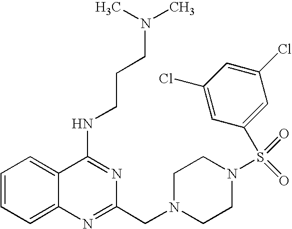 | 537.5 | 537.3 |
| 27-40 | 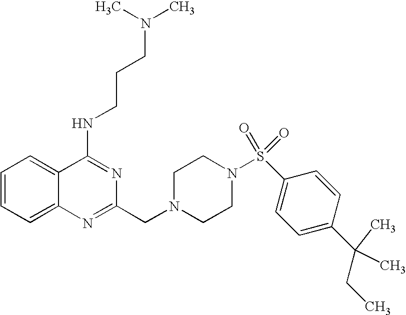 | 538.8 | 539.3 |
| 27-41 | 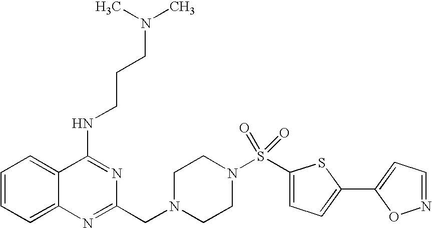 | 541.7 | 542.3 |
| 27-42 | 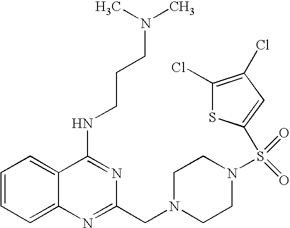 | 543.5 | 543.3 |
| 27-43 | 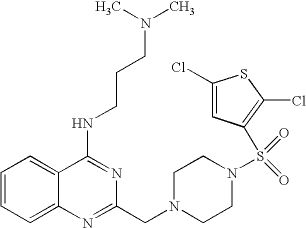 | 543.5 | 543.3 |
| 27-44 | 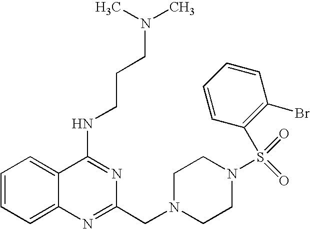 | 547.5 | 549.3 |
| 27-45 | 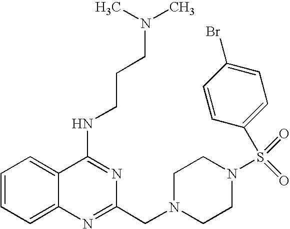 | 547.5 | 549.3 |
| 27-46 | 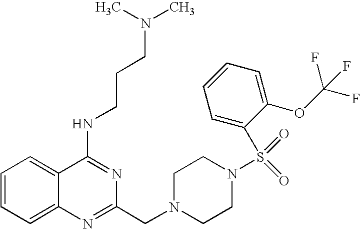 | 552.6 | 553.3 |
| 27-47 | 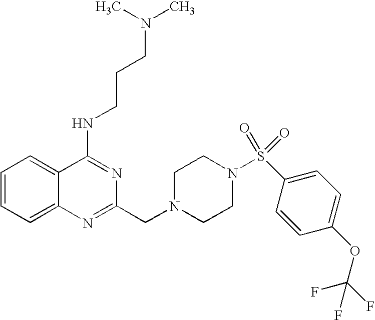 | 552.6 | 553.3 |
| 27-48 | 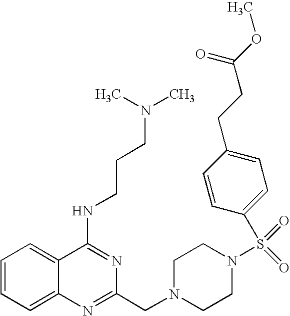 | 554.7 | 555.3 |
| 27-49 | 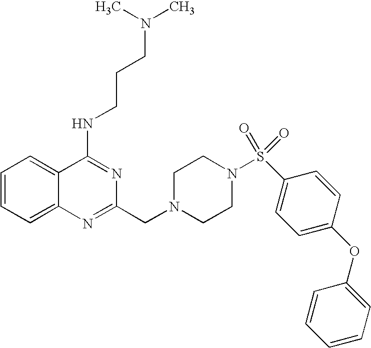 | 560.7 | 561.3 |
| 27-50 | 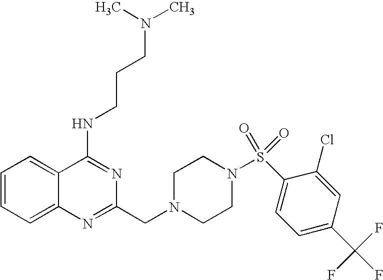 | 571.1 | 571.3 |
| 27-51 | 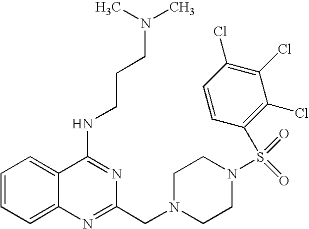 | 572.0 | 573.3 |
| 27-52 | 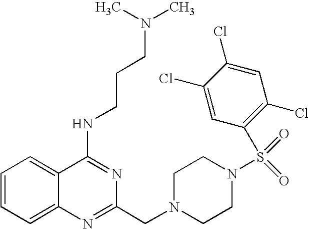 | 572.0 | 573.3 |
| 27-53 | 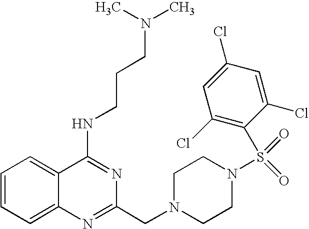 | 572.0 | 573.3 |
| 27-54 | 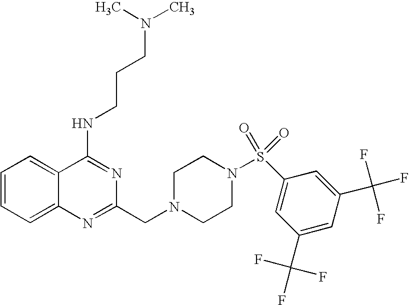 | 604.6 | 605.3 |
| 27-55 | 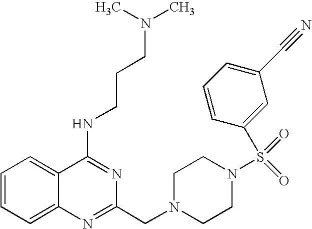 | 493.6 | 494.3 |
| 27-56 | 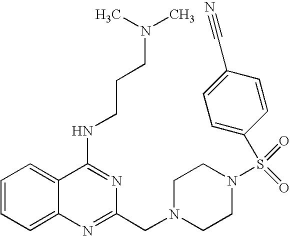 | 493.6 | 494.3 |
| 27-57 | 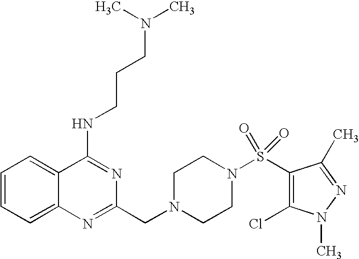 | 521.1 | 521.3 |
| 27-58 | 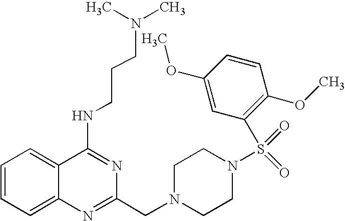 | 528.7 | 529.3 |
| 27-59 | 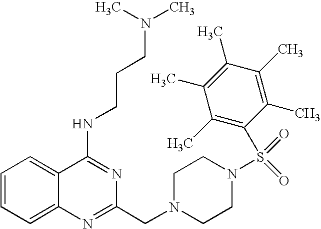 | 538.8 | 539.3 |
| 27-60 | 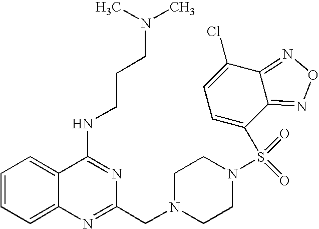 | 545.1 | 545.3 |
| 27-61 | 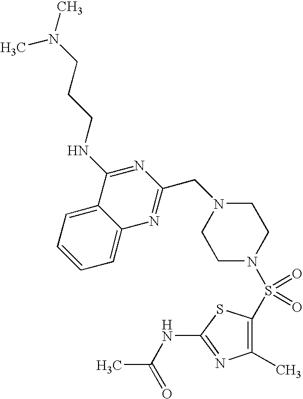 | 546.7 | 547.3 |
| 27-62 | 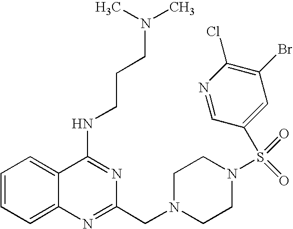 | 583.0 | 584.3 |
| 27-63 | 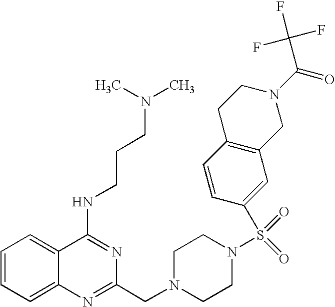 | 619.7 | 620.3 |
| 27-64 | 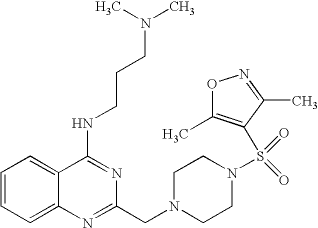 | 487.6 | 488.3 |
| 27-65 | 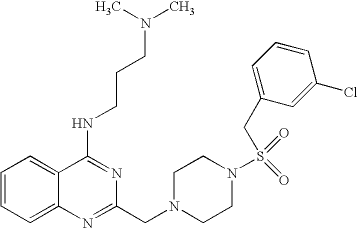 | 517.1 | 517.3 |
| 27-66 | 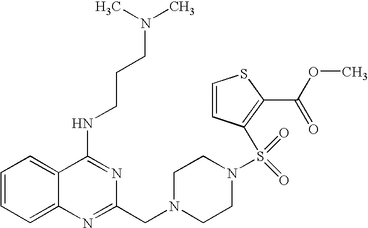 | 532.7 | 533.3 |
| 27-67 | 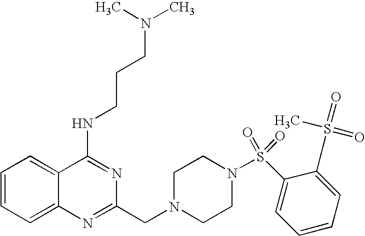 | 546.7 | 547.3 |
| 27-68 | 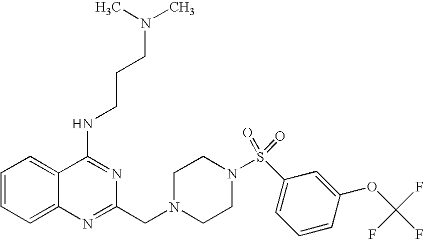 | 552.6 | 553.3 |
| 27-69 | 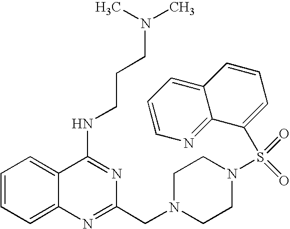 | 519.7 | 520.3 |
| 27-70 | 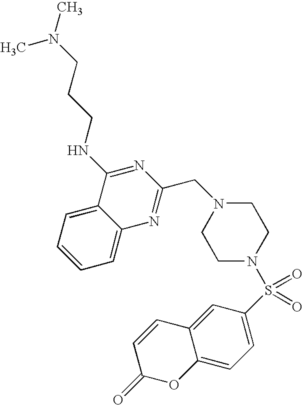 | 536.7 | 537.3 |
| 27-71 | 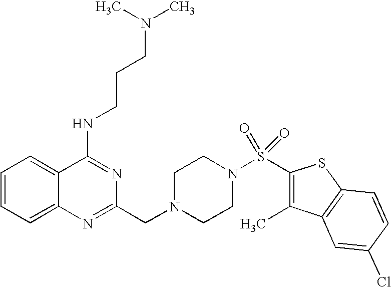 | 573.2 | 573.3 |
| 27-72 | 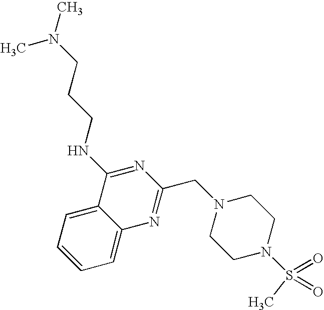 | 406.6 | 407.2 |
| 27-73 | 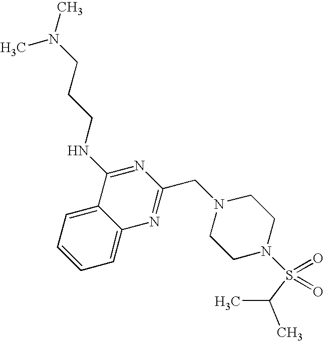 | 434.6 | 435.2 |
| 27-74 | 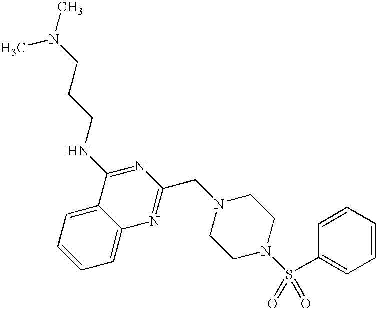 | 468.6 | 469.3 |
| 27-75 | 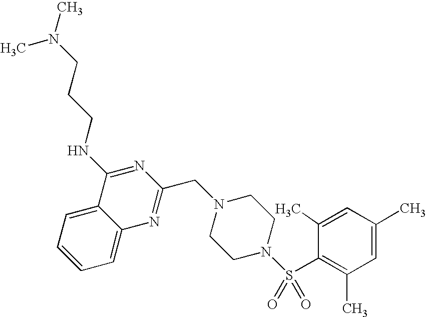 | 510.7 | 511.3 |

### Example 28

### Reaction of N,N-DIMETHYL-N′-[2-(PIPERAZIN-1-YLMETHYL)QUINAZOLIN-4-YL]PROPANE-1,3-DIAMINE with an ISOCYANATE LIBRARY

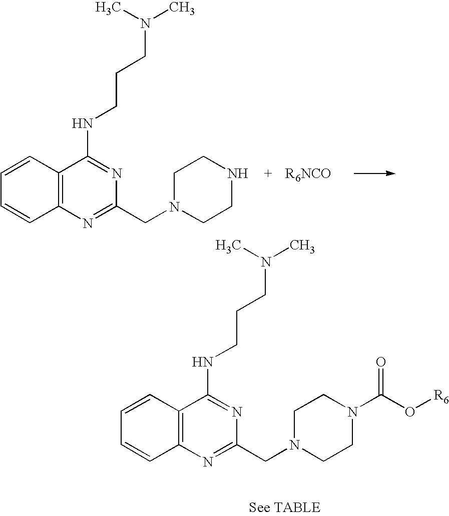

A stock solution of 2(S)-[2-(piperazin-1-ylmethyl)quinazolin-4-ylamino]-3-methylbutyramide (1 mL, 0.0233 mmoles) (prepared as described in Preparative Example 24 above) in DCE was added to the tubes in three 24 tube Bohdan Miniblocks. A 1M stock solution of each of the isocyanates (R₆NCO) in THF (0.05 mL, 0.0466 mmoles) was added to each tube. PS-Isocyanate resin (3 equivalents, 0.0699 mmoles) was added to each tube, followed by PS-Trisamine resin (6 equivalents, 0.14 mmoles) and the Miniblocks were shaken at 25° C. for 18 h. The blocks were drained onto MP-TsOH resin (4 equivalents, 0.0932 mmoles) and the PS-Trisamine resins were washed with DCE. The Miniblocks were shaken at 25° C. for 4 h and then drained and the resin was washed with dichloromethane and the filtrates were discarded. Ammonia in methanol (2N, 2 mL) was added to each tube and the Miniblocks were shaken at 25° C. for 4 h. The methanol filtrates were collected and the resin was again shaken with ammonia in methanol (2N, 2 mL). The combined filtrates from each tube were evaporated to dryness overnight, on a Speedvac concentrator. The resulting samples were analyzed by LCMS and those that were >70% pure are listed in the table below.

### Examples 28-1 Through 28-48

| | | | | | SPA |
|---|---|---|---|---|---|
| | | | | | ASSAY |
| | | | | | (% |
| | | | | PROFILE | Residual |
| | | | | RATION | T @ |
| | | | | ASSAY | 2 |
| | | | | EC50 | ug/mL |
| | | | LCMS | MB168 | of drug |
| EXAMPLE | STRUCTURE | MW | m/z | (uM) | (average of) |
| 28-1 | 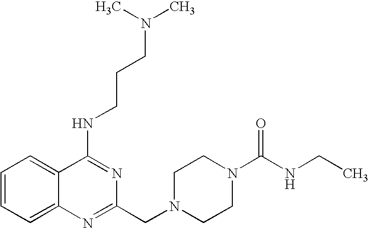 | 399.5 | 400.2 | | |
| 28-2 | 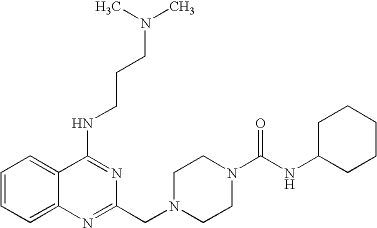 | 453.6 | 454.2 | | |
| 28-3 | 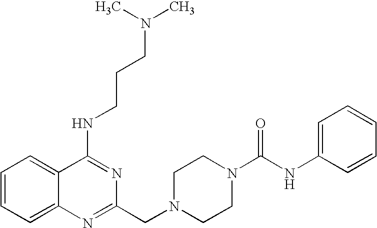 | 447.6 | 448.2 | | |
| 28-4 | 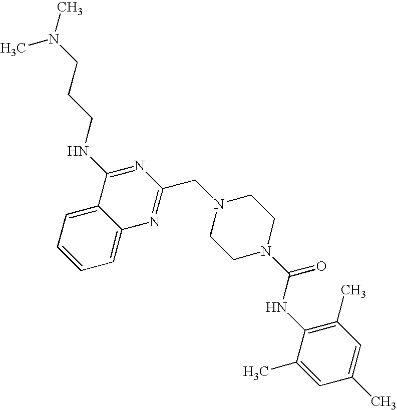 | 489.7 | 490.3 | C | C |
| 28-5 | 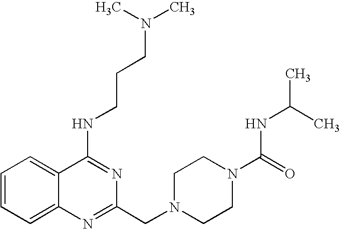 | 413.6 | 414.2 | | |
| 28-6 | 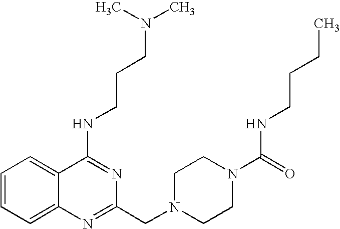 | 427.6 | 428.2 | | |
| 28-7 | 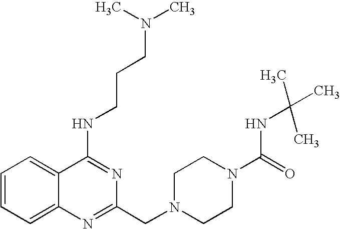 | 427.6 | 428.2 | | |
| 28-8 | 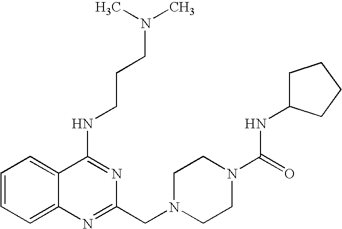 | 439.6 | 440.2 | | |
| 28-9 | 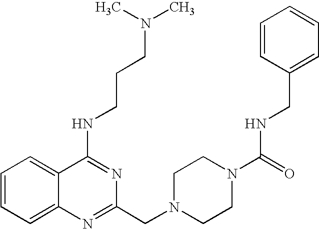 | 461.6 | 462.3 | | |
| 28-10 | 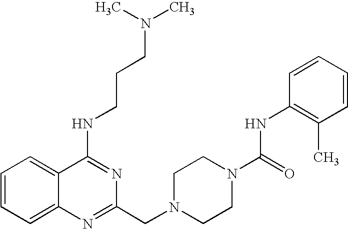 | 461.6 | 462.3 | | |
| 28-11 | 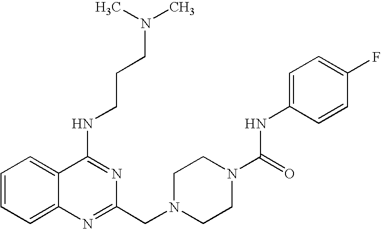 | 465.6 | 466.3 | | |
| 28-12 | 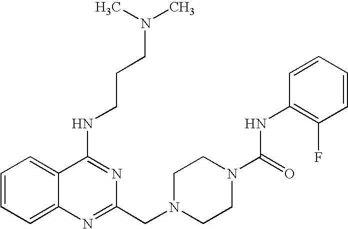 | 465.6 | 466.3 | | |
| 28-13 | 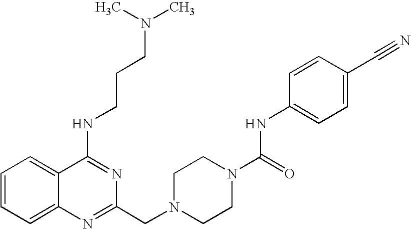 | 472.6 | 473.3 | D | C |
| 28-14 | 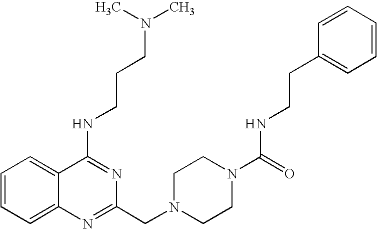 | 475.6 | 476.3 | | |
| 28-15 | 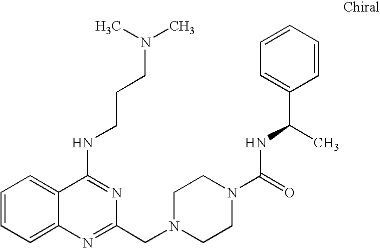 | 475.6 | 476.3 | | |
| 28-16 | 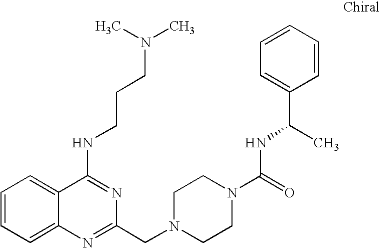 | 475.6 | 476.3 | | |
| 28-17 | 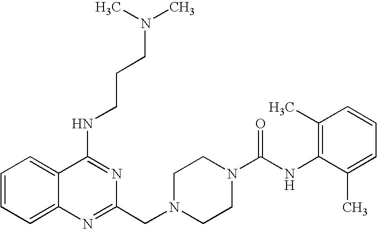 | 475.6 | 476.3 | | |
| 28-18 | 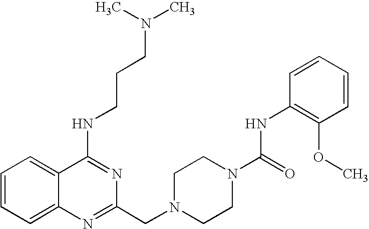 | 477.6 | 478.3 | | |
| 28-19 | 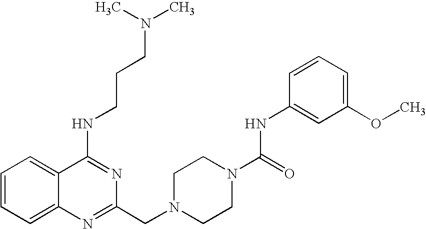 | 477.6 | 478.3 | | |
| 28-20 | 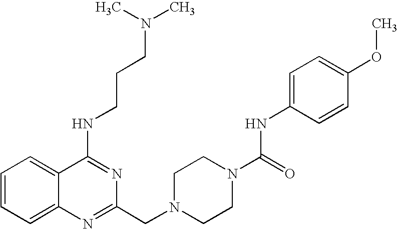 | 477.6 | 478.3 | D | B |
| 28-21 | 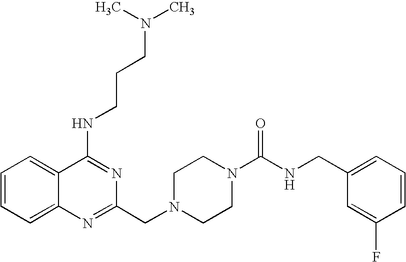 | 479.6 | 480.3 | | |
| 28-22 | 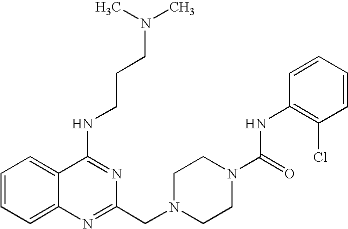 | 482.0 | 482.3 | | |
| 28-23 | 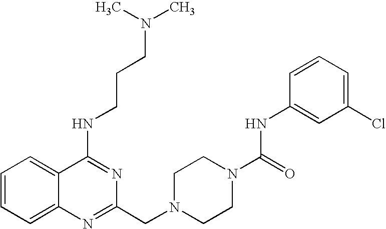 | 482.0 | 482.3 | | |
| 28-24 | 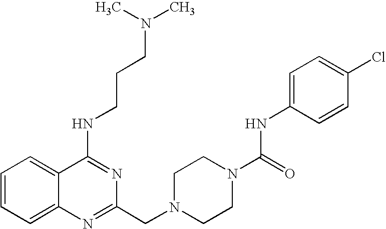 | 482.0 | 482.3 | | |
| 28-25 | 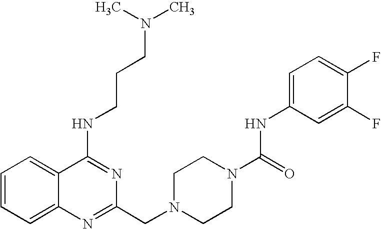 | 483.6 | 484.3 | | |
| 28-26 | 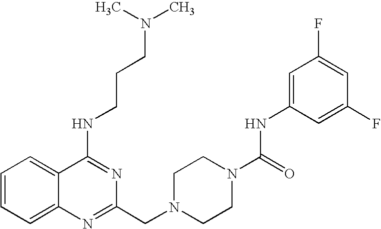 | 483.6 | 484.3 | | |
| 28-27 | 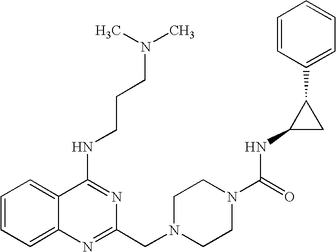 | 487.7 | 488.3 | | |
| 28-28 | 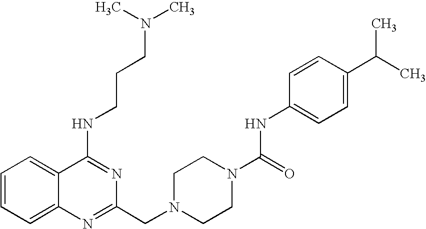 | 489.7 | 490.3 | | |
| 28-29 | 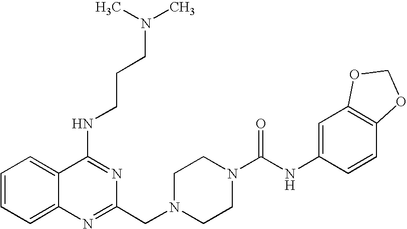 | 491.6 | 492.3 | | |
| 28-30 | 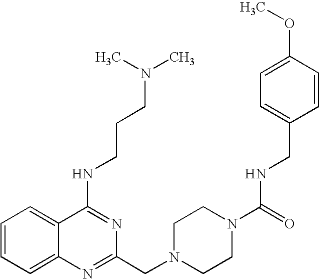 | 491.6 | 492.3 | | |
| 28-31 | 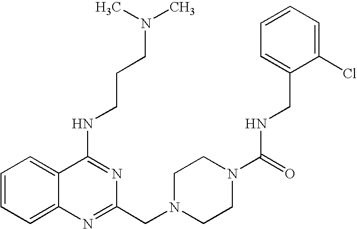 | 496.1 | 496.3 | | |
| 28-32 | 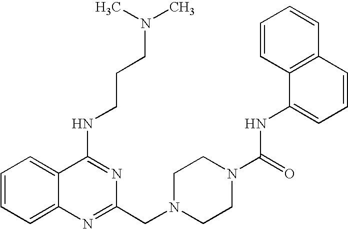 | 497.6 | 498.3 | | |
| 28-33 | 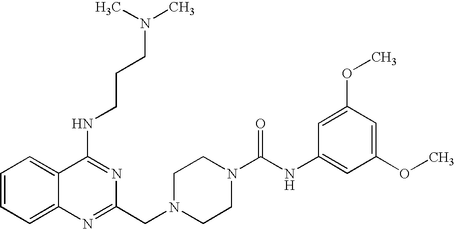 | 507.6 | 508.3 | | |
| 28-34 | 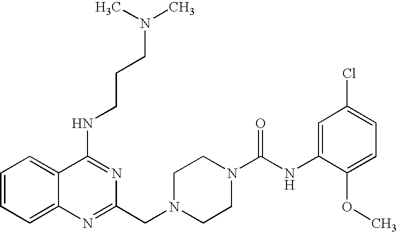 | 512.1 | 512.3 | | |
| 28-35 | 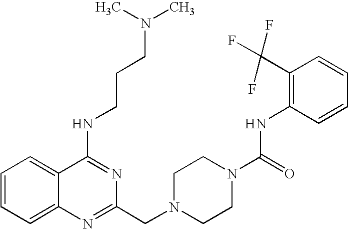 | 515.6 | 516.3 | | |
| 28-36 | 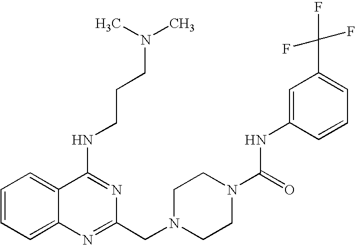 | 515.6 | 516.3 | | |
| 28-37 | 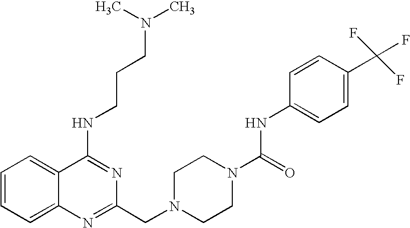 | 515.6 | 516.3 | | |
| 28-38 | 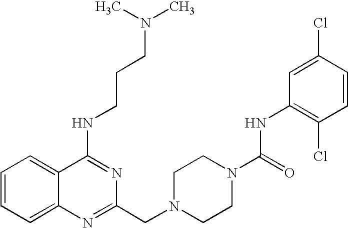 | 516.5 | 516.3 | | |
| 28-39 | 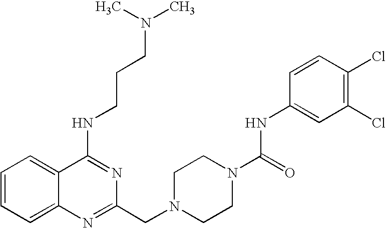 | 516.5 | 516.3 | C | B |
| 28-40 | 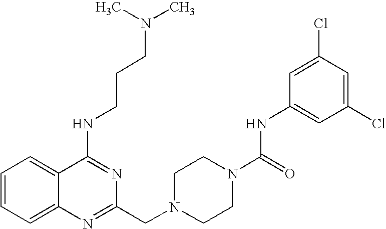 | 516.5 | 516.3 | | |
| 28-41 | 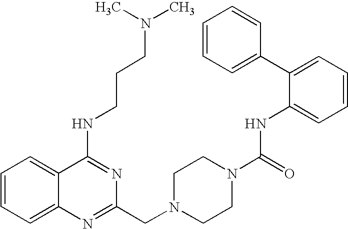 | 523.7 | 524.3 | | |
| 28-42 | 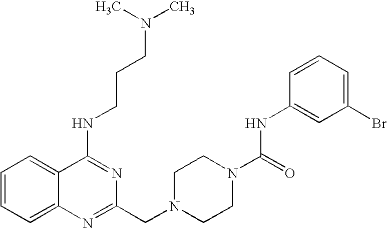 | 526.5 | 528.3 | | |
| 28-43 | 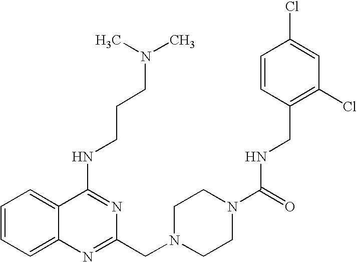 | 530.5 | 530.3 | | |
| 28-44 | 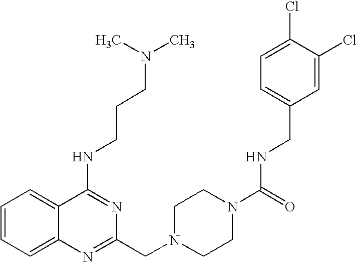 | 530.5 | 530.3 | | |
| 28-45 | 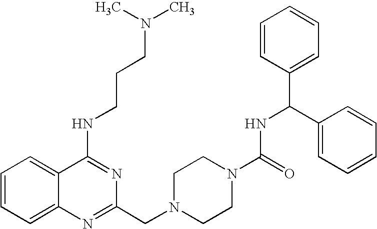 | 537.7 | 538.3 | | |
| 28-46 | 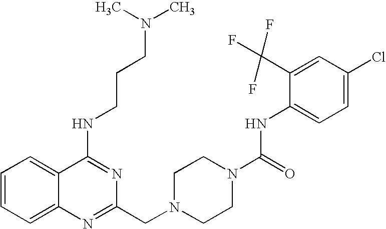 | 550.0 | 550.3 | | |
| 28-47 | 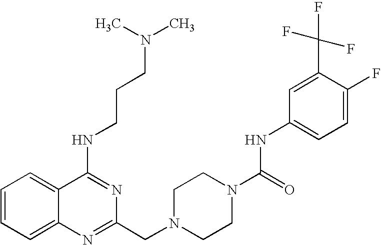 | 533.6 | 534.3 | | |
| 28-48 | 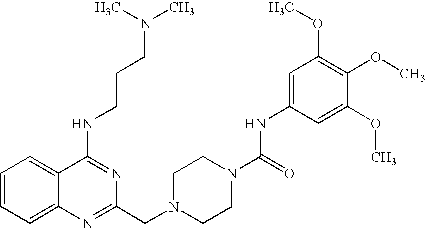 | 537.7 | 538.3 | | |

### Example 29

### Reaction of 2-{4-[BIS-(4-CHLOROPHENYL)METHYL]PIPERAZIN-1-YLMETHYL}-4-CHLOROQUINAZOLINE with a PRIMARY AMINE LIBRARY

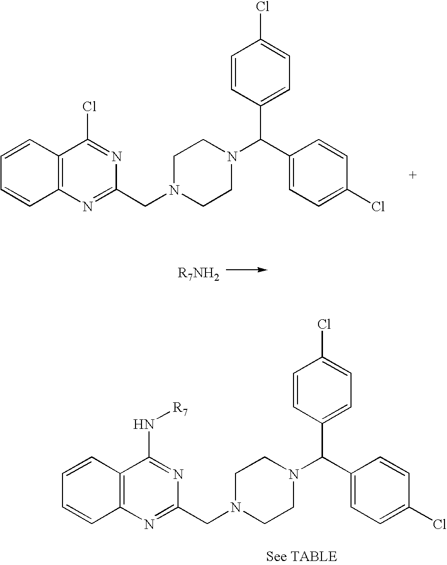

A slurry of PS-DMAP resin (28 mg, 0.04 mmoles for free primary amines; 84 mg, 0.12 mmoles for primary amine hydrochlorides) in anhydrous 1,4-dioxane was introduced into each tube of a heated 96 well shaker block containing anhydrous 1,4-dioxane (1 ml) in each tube. A stock solution of 2-{4-[bis-(4-chlorophenyl)methyl]piperazin-1-ylmethyl}-4-chloroquinazoline (1 mL, 0.02 mmoles) (prepared as described in Preparative Example 8 above) in anhydrous 1,4-dioxane was added to each tube. A 1M stock solution of each of the individual primary amines (R₇NH₂) (30 μL, 0.03 mmoles) in THF was added to each tube. The block was sealed and heated at 60° C. for 72 h. The block was allowed to cool to 25° C. and the volume of solvent in each tube was brought up to ˜2 ml with anhydrous 1,4-dioxane where needed. PS-Isocyanate resin (81.6 mg, 0.12 mmoles0 was added to each tube and the block was re-sealed and shaken at 25° C. for 17 h. The solvent was filtered off into suitable vials and the resin was washed with THF (2 mL/well). The combined filtrates were evaporated to dryness on a Speedvac concentrator. The resulting samples were analyzed by LCMS and any samples that were <70% pure, were further purified by preparative LCMS. The samples were each dissolved in 60% DMSO-acetonitrile (samples >16.9 mg in 1.5 mL; samples <16.98 mg in 0.8 mL) and 0.8 mL of each were injected onto the preparative HPLC (using a Phenomenex Luna 5 n C-18(2) column; 60×21.2 mm; 5 n micron; flow rate of 20 mL/min; gradient elution using water-acetonitrile-1% aqueous formic acid) and the fractions corresponding to the desired molecular weight of the product+/−1 mu were collected. Where the quantity of material was >0.8 mL, multiple injections of 0.8 ml were made to afford additional compound. The compounds that were prepared having a purity >70%, are listed in the table below. Using similar procedures to those described for Examples 29-1 through 29-26, the compounds of Examples 29-27 through 29-67 may also be prepared.

### Examples 29-1 Through 29-67

| EXAMPLE | STRUCTURE | MW | LCMS m/z |
|---|---|---|---|
| 29-1 | 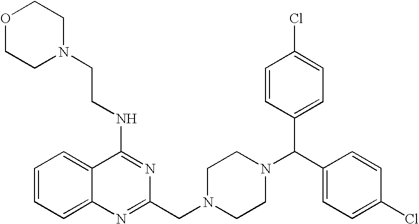 | 591.6 | 591.1 |
| 29-2 | 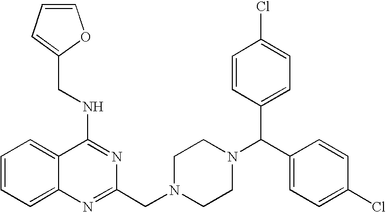 | 558.5 | 558.1 |
| 29-3 | 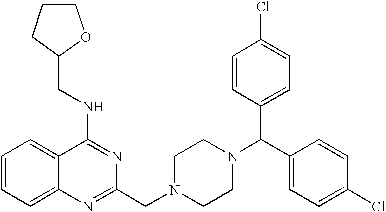 | 562.5 | 562.1 |
| 29-4 | 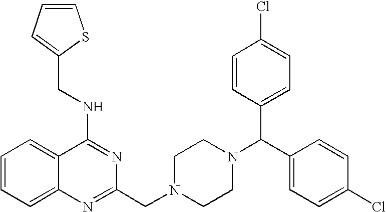 | 574.6 | 574.1 |
| 29-5 | 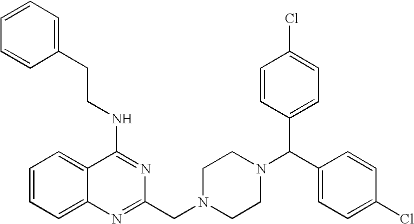 | 582.6 | 582.1 |
| 29-6 | 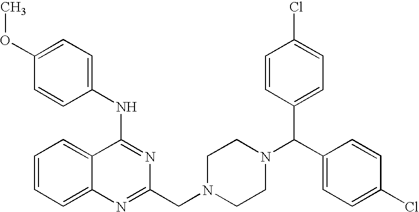 | 584.6 | 584.1 |
| 29-7 | 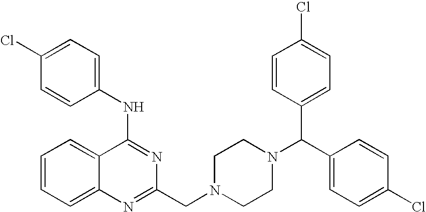 | 589.0 | 588.1 |
| 29-8 | 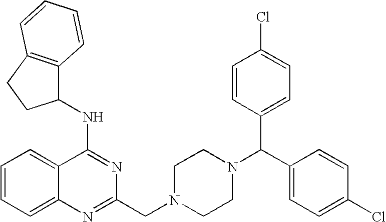 | 594.6 | 594.1 |
| 29-9 | 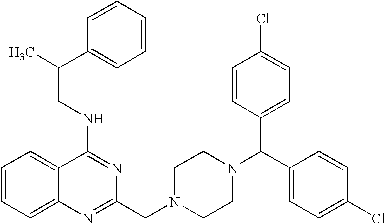 | 596.6 | 596.1 |
| 29-10 | 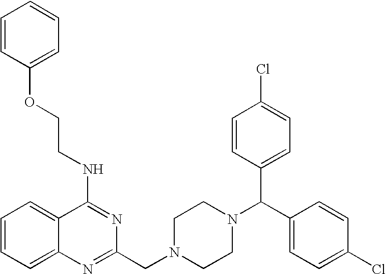 | 598.6 | 598.1 |
| 29-11 | 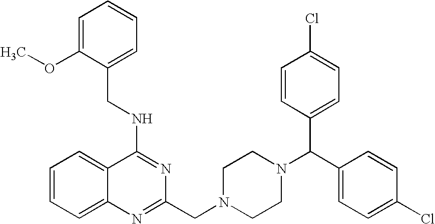 | 598.6 | 598.1 |
| 29-12 | 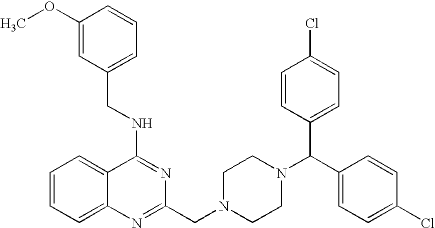 | 598.6 | 598.1 |
| 29-13 | 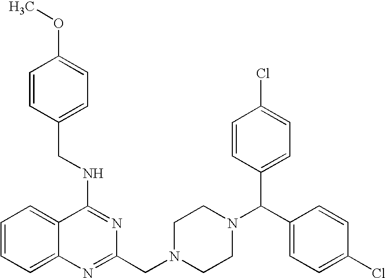 | 598.6 | 598.1 |
| 29-14 | 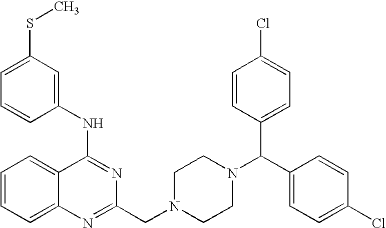 | 600.6 | 600.1 |
| 29-15 | 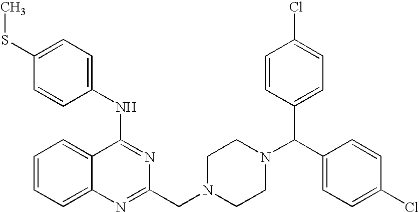 | 600.6 | 600.1 |
| 29-16 | 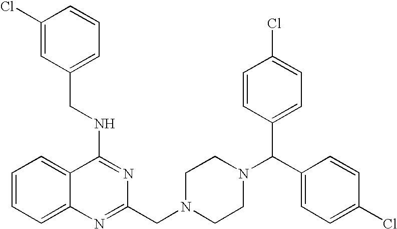 | 603.0 | 604.1 |
| 29-17 | 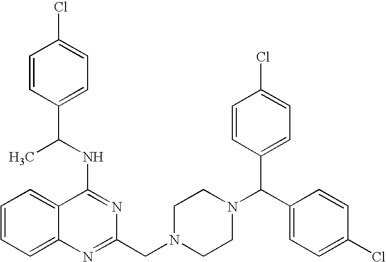 | 617.0 | 617.1 |
| 29-18 | 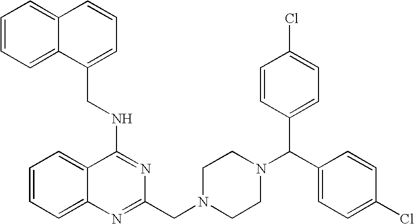 | 618.6 | 618.1 |
| 29-19 | 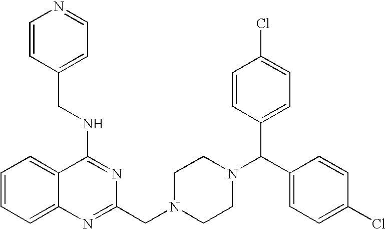 | 569.5 | 569.1 |
| 29-20 | 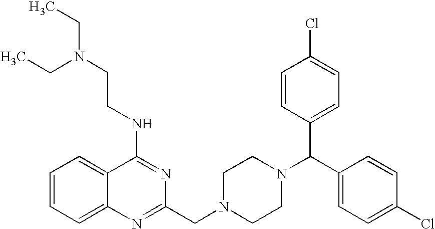 | 577.6 | 577.1 |
| 29-21 | 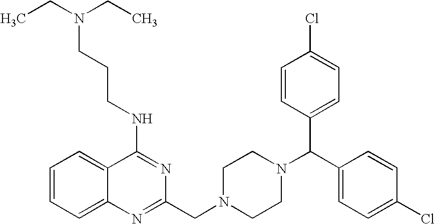 | 591.6 | 591.1 |
| 29-22 | 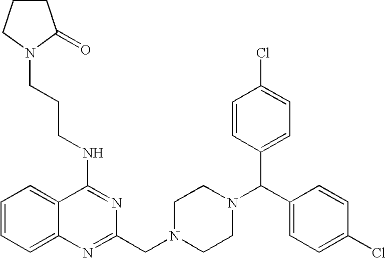 | 603.6 | 603.1 |
| 29-23 | 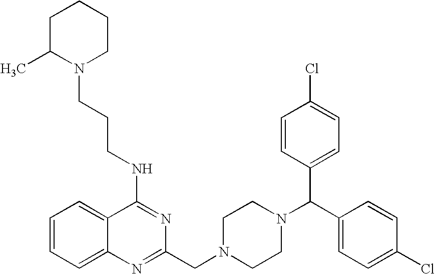 | 617.7 | 617.1 |
| 29-24 | 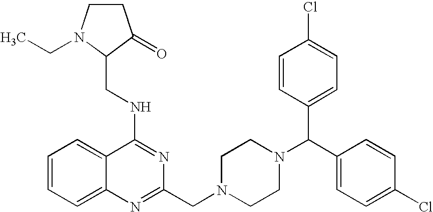 | 589.6 | 589.1 |
| 29-25 | 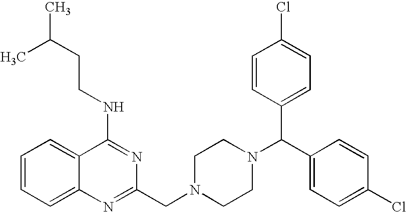 | 548.6 | 548.1 |
| 29-26 | 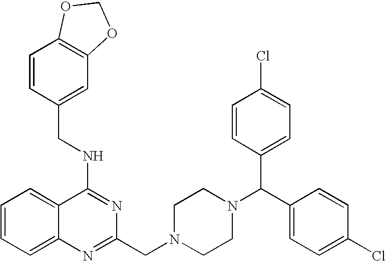 | 612.6 | 612.6 |
| 29-27 | 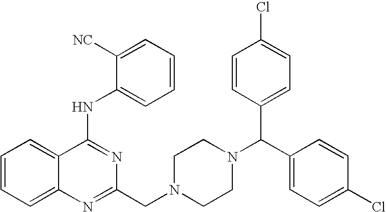 | | |
| 29-28 | 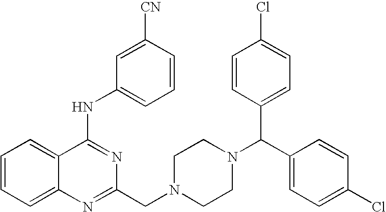 | | |
| 29-29 | 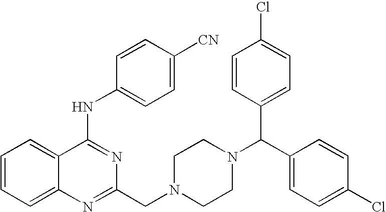 | | |
| 29-30 | 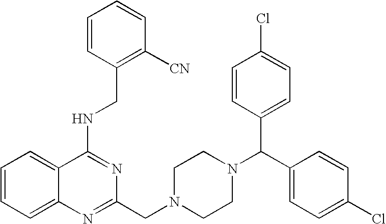 | | |
| 29-31 | 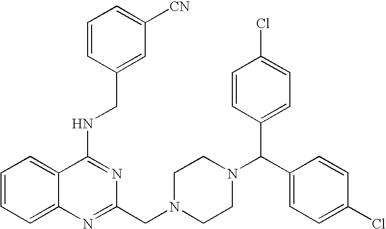 | | |
| 29-32 | 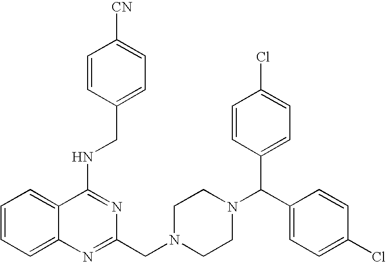 | | |
| 29-33 | 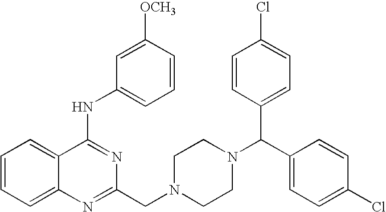 | | |
| 29-34 | 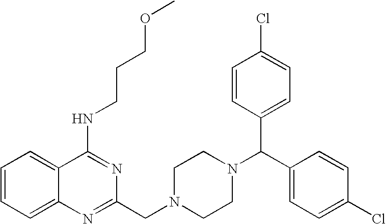 | | |
| 29-35 | 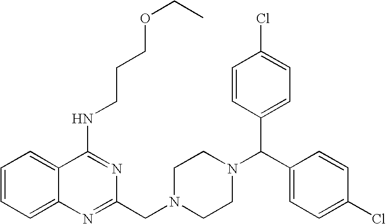 | | |
| 29-36 | 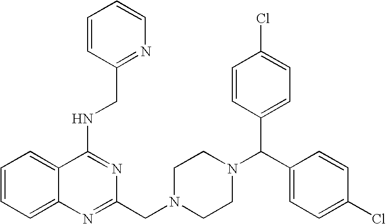 | | |
| 29-37 | 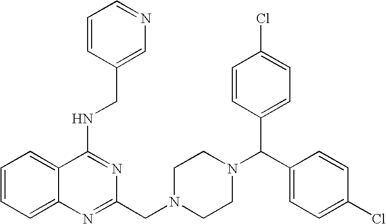 | | |
| 29-38 | 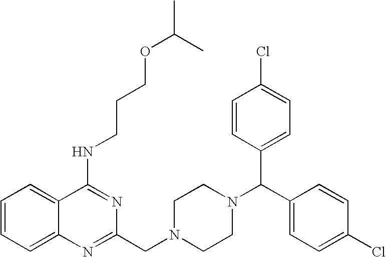 | | |
| 29-39 | 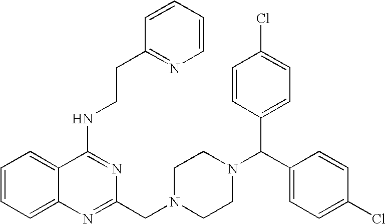 | | |
| 29-40 | 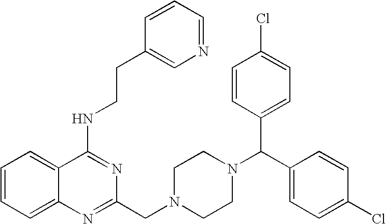 | | |
| 29-41 | 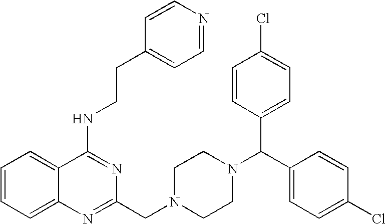 | | |
| 29-42 | 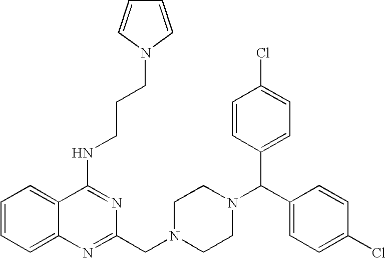 | | |
| 29-43 | 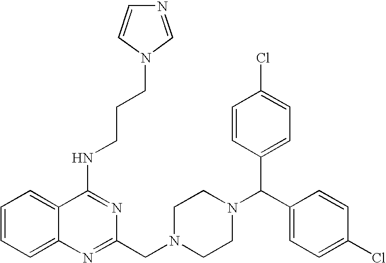 | | |
| 29-44 | 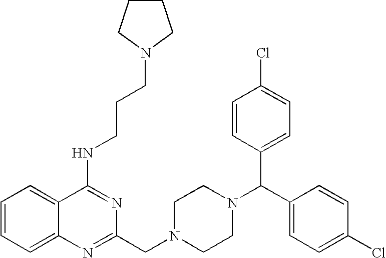 | | |
| 29-45 | 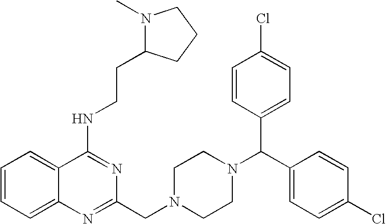 | | |
| 29-46 | 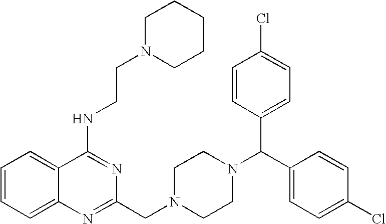 | | |
| 29-47 | 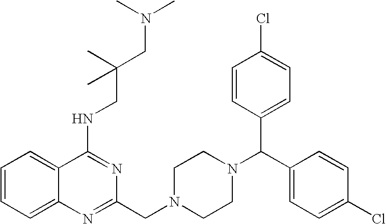 | | |
| 29-48 | 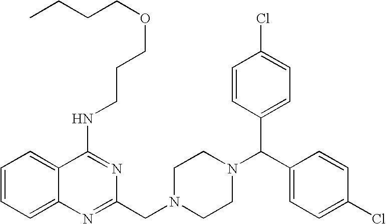 | | |
| 29-49 | 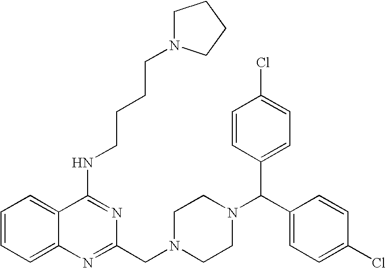 | | |
| 29-50 | 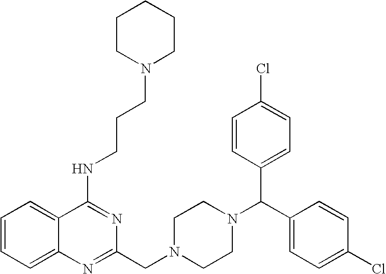 | | |
| 29-51 | 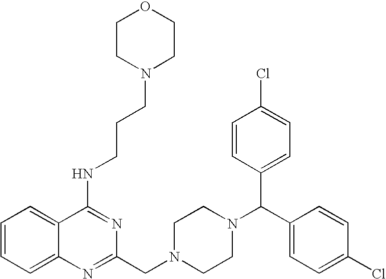 | | |
| 29-52 | 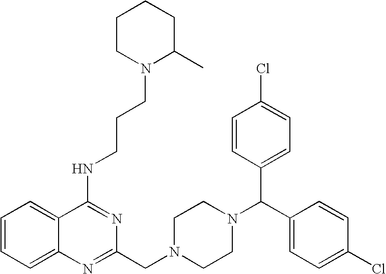 | | |
| 29-53 | 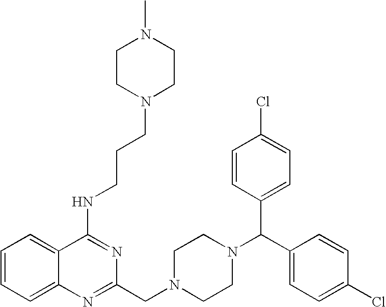 | | |
| 29-54 | 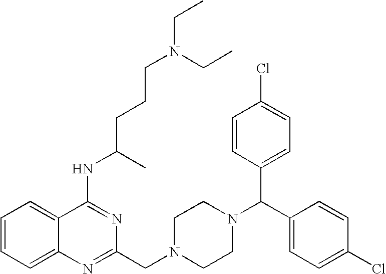 | | |
| 29-55 | 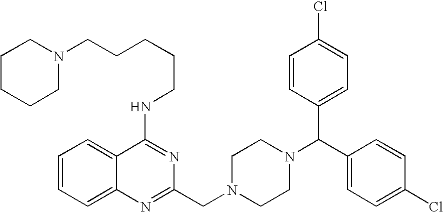 | | |
| 29-56 | 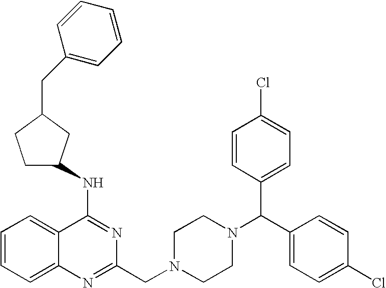 | | |
| 29-57 | 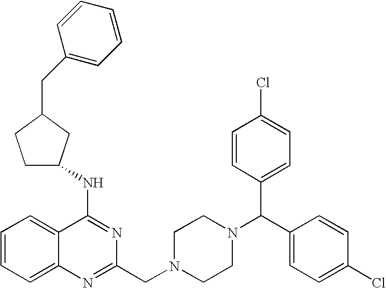 | | |
| 29-58 | 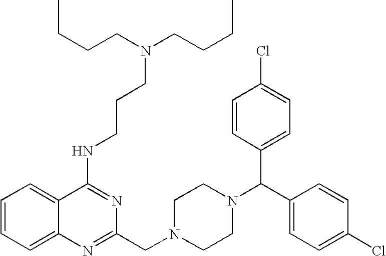 | | |
| 29-59 | 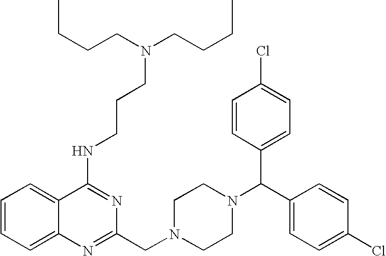 | | |
| 29-60 | 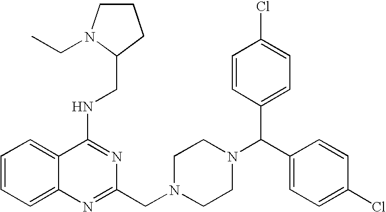 | | |
| 29-61 | 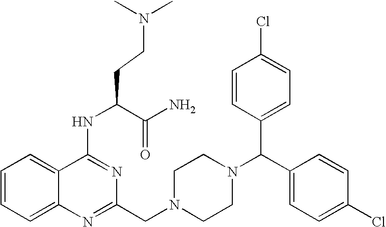 | | |
| 29-62 | 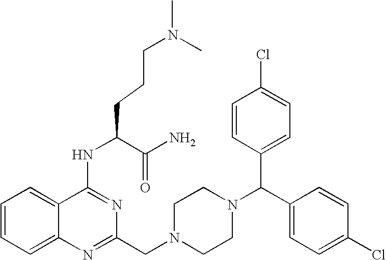 | | |
| 29-63 | 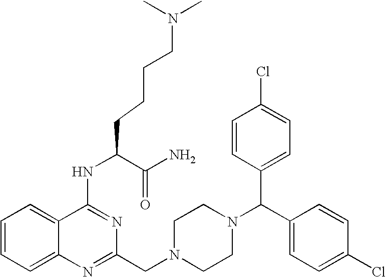 | | |
| 29-64 | 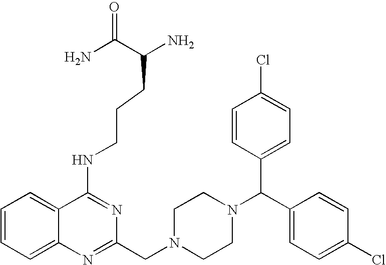 | | |
| 29-65 | 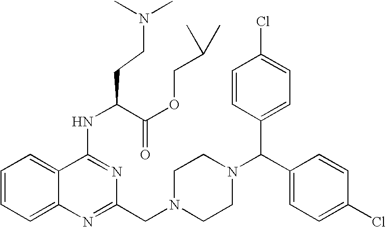 | | |
| 29-66 | 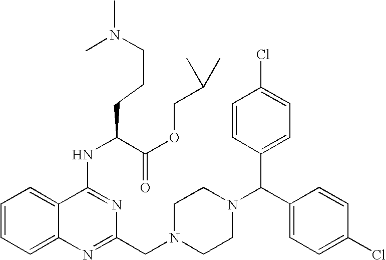 | | |
| 29-67 | 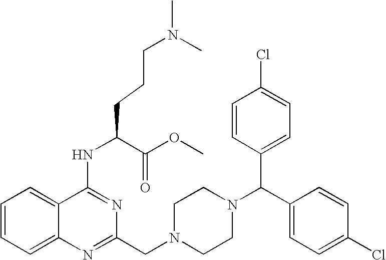 | | |

### Example 30

### Reaction of 2-{4-[BIS-(4-CHLOROPHENYL)METHYL]PIPERAZIN-1-YLMETHYL}-4-CHLOROQUINAZOLINE with a SECONDARY AMINE LIBRARY

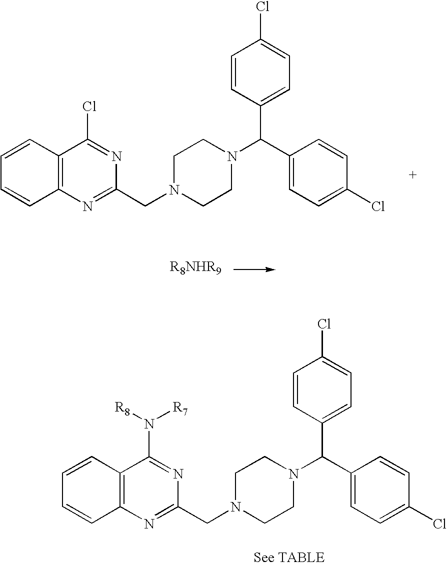

A slurry of PS-DMAP resin (28 mg, 0.04 mmoles for free primary amines; 84 mg, 0.12 mmoles for primary amine hydrochlorides) in anhydrous 1,4-dioxane was introduced into each tube of a heated 96 well shaker block containing anhydrous 1,4-dioxane (1 ml) in each tube. A stock solution of 2-{4-[bis-(4-chlorophenyl)methyl]piperazin-1-ylmethyl}-4-chloroquinazoline (1 mL, 0.02 mmoles) (prepared as described in Preparative Example 8 above) in anhydrous 1,4-dioxane was added to each tube. A 1M stock solution of each of the individual secondary amines (R₈NHR₉) (30 μL, 0.03 mmoles) in THF was added to each tube. The block was sealed and heated at 60° C. for 72 h. The block was allowed to cool to 25° C. and the volume of solvent in each tube was brought up to ˜2 ml with anhydrous 1,4-dioxane where needed. PS-Isocyanate resin (81.6 mg, 0.12 mmoles0 was added to each tube and the block was re-sealed and shaken at 25° C. for 17 h. The solvent was filtered off into suitable vials and the resin was washed with THF (2 mL/well). The combined filtrates were evaporated to dryness on a Speedvac concentrator. The resulting samples were analyzed by LCMS and any samples that were <70% pure, were further purified by preparative LCMS. The samples were each dissolved in 60% DMSO-acetonitrile (1 mL) and 0.8 mL of each were injected onto the preparative HPLC (using a Phenomenex Luna 5 n C-18(2) column; 60×21.2 mm; 5 n micron; flow rate of 20 mL/min; gradient elution using water-acetonitrile-1% aqueous formic acid) and the fractions corresponding to the desired molecular weight of the product+/−1 mu were collected. Where the quantity of material was >0.8 mL, multiple injections of 0.8 ml were made to afford additional compound. The compounds that were prepared having a purity >70%, are listed in the table below. Using similar procedures to those described above for Examples 30-1 through 30-30, the compounds of Examples 30-31 through 30-58 may also be prepared.

### Examples 30-1 Through 30-58

| | | | | PROLI- | SPA |
|---|---|---|---|---|---|
| | | | | FERAT | ASSAY |
| | | | | ION | (% |
| | | | | ASSAY | residual |
| | | | | EC50 | T @ 2 |
| | | | LCMS | MB468 | ug/mL of |
| EXAMPLE | STRUCTURE | MW | m/z | (uM) | drug |
| 30-1 | 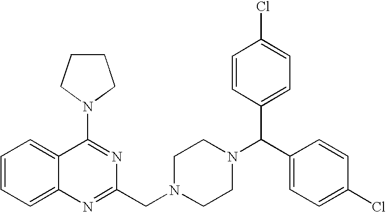 | 532.5 | 532.1 | | |
| 30-2 | 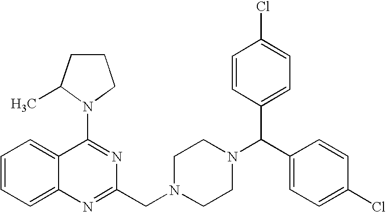 | 546.5 | 546.1 | | |
| 30-3 | 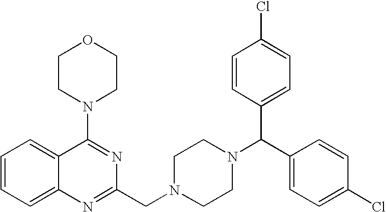 | 548.5 | 548.1 | D | D |
| 30-4 | 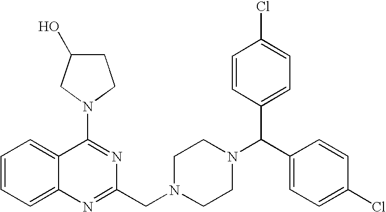 | 548.5 | 548.1 | D | D |
| 30-5 | 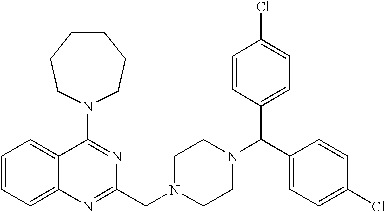 | 560.6 | 560.1 | B | |
| 30-6 | 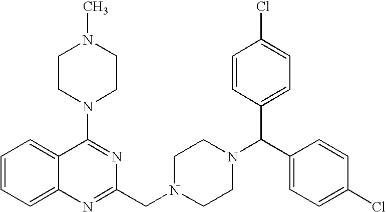 | 561.6 | 561.1 | | |
| 30-7 | 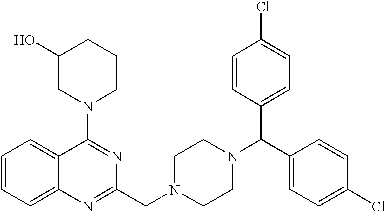 | 562.5 | 562.1 | | |
| 30-8 | 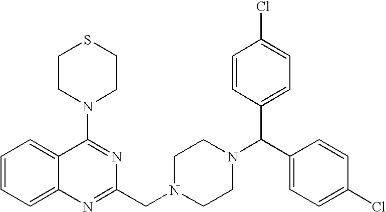 | 564.6 | 564.1 | | |
| 30-9 | 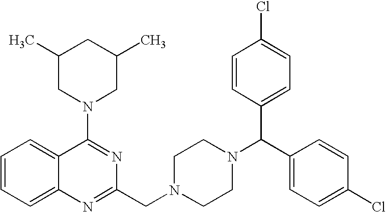 | 574.6 | 574.1 | | |
| 30-10 | 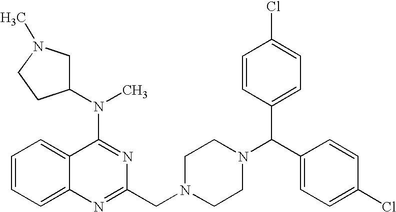 | 575.6 | 574.1 | | |
| 30-11 | 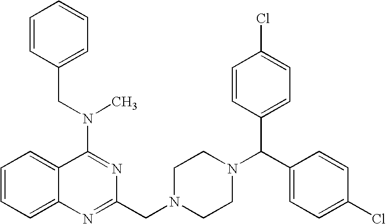 | 582.6 | 582.1 | D | E |
| 30-12 | 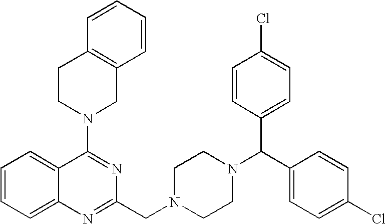 | 594.6 | 594.1 | D | E |
| 30-13 | 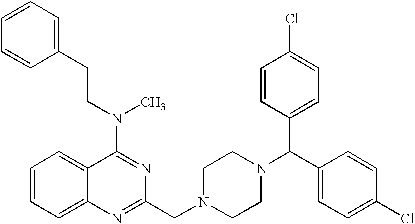 | 596.6 | 596.1 | | |
| 30-14 | 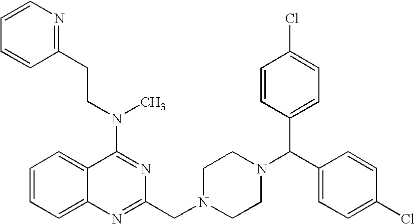 | 597.6 | 597.1 | C | D |
| 30-15 | 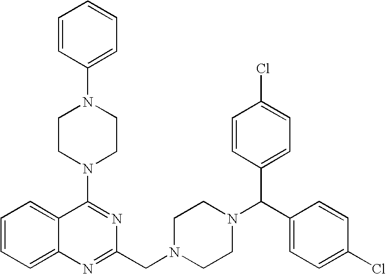 | 623.6 | 623.1 | C | E |
| 30-16 | 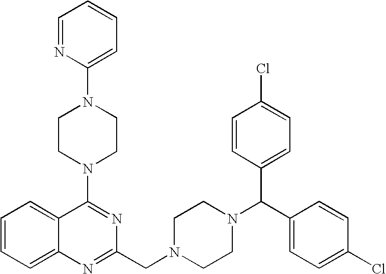 | 624.6 | 624.1 | | |
| 30-17 | 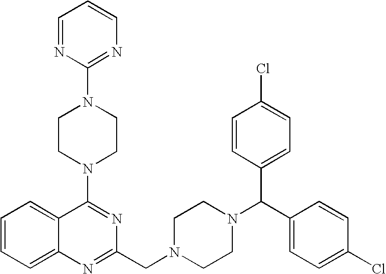 | 625.6 | 625.1 | D | E |
| 30-18 | 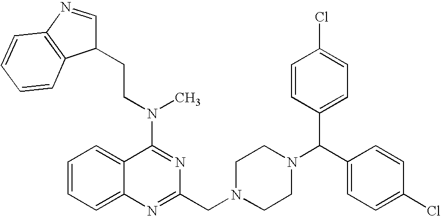 | 635.6 | 635.2 | B | |
| 30-19 | 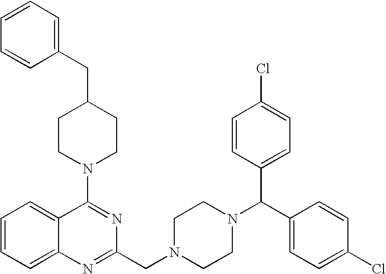 | 636.7 | 636.2 | C | |
| 30-20 | 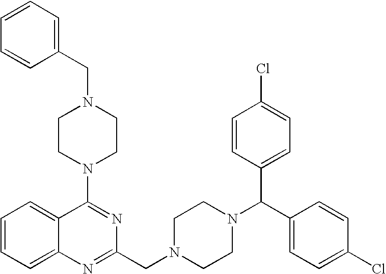 | 637.7 | 636.2 | B | |
| 30-21 | 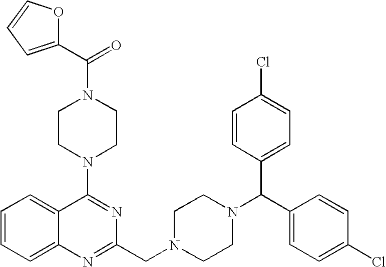 | 641.6 | 641.2 | | |
| 30-22 | 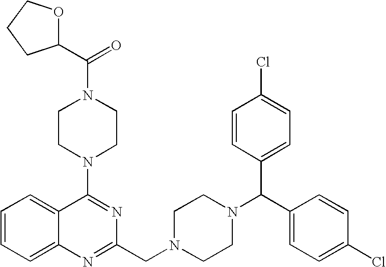 | 645.6 | 645.2 | D | D |
| 30-23 | 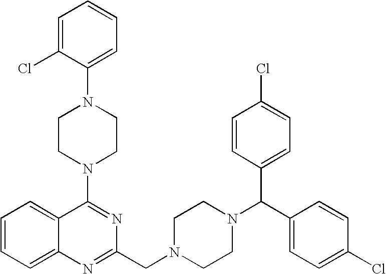 | 658.1 | 659.2 | | |
| 30-24 | 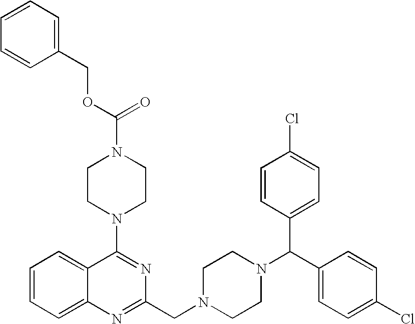 | 681.7 | 681.2 | | |
| 30-25 | 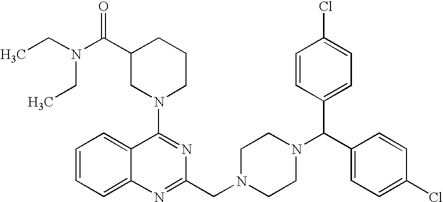 | 645.7 | 645.2 | D | D |
| 30-26 | 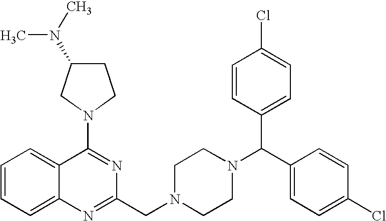 | 575.6 | 575.1 | D | C |
| 30-27 | 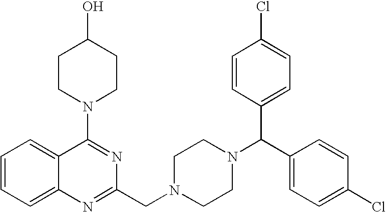 | 562.5 | 562.1 | C | D |
| 30-28 | 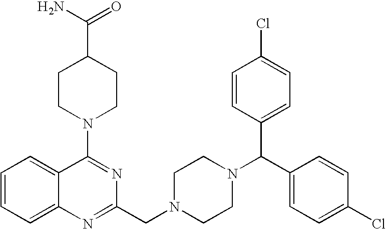 | 589.6 | 589.1 | D | D |
| 30-29 | 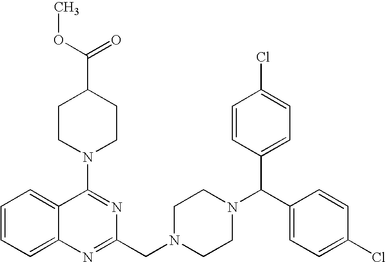 | 604.6 | 604.1 | C | D |
| 30-30 | 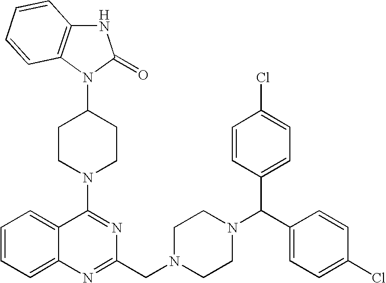 | 678.7 | 678.2 | C | C |
| 30-31 | 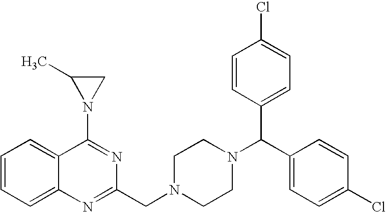 | | | | |
| 30-32 | 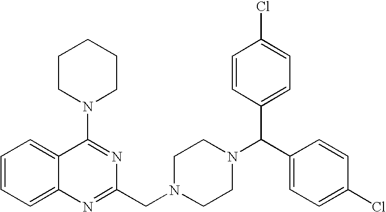 | | | | |
| 30-33 | 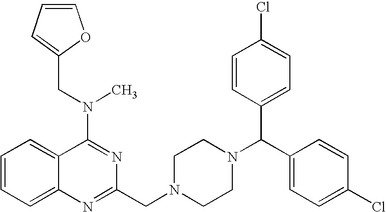 | | | | |
| 30-34 | 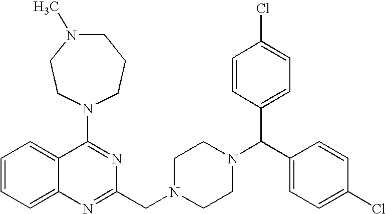 | | | | |
| 30-35 | 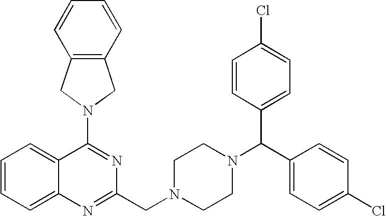 | | | | |
| 30-36 | 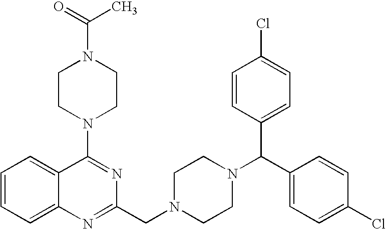 | | | | |
| 30-37 | 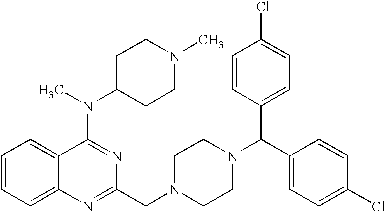 | | | | |
| 30-38 | 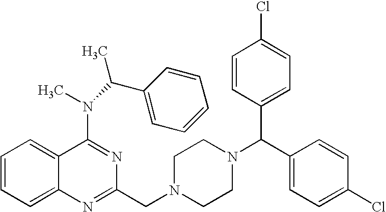 | | | | |
| 30-39 | 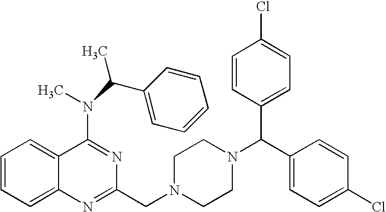 | | | | |
| 30-40 | 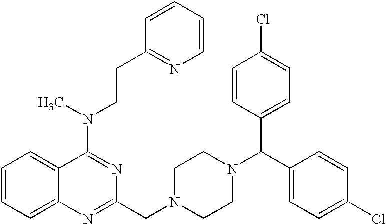 | | | | |
| 30-41 | 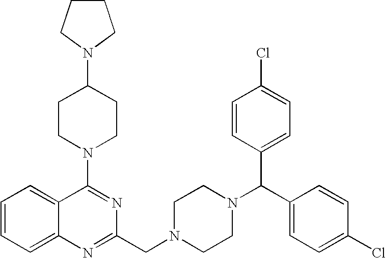 | | | | |
| 30-42 | 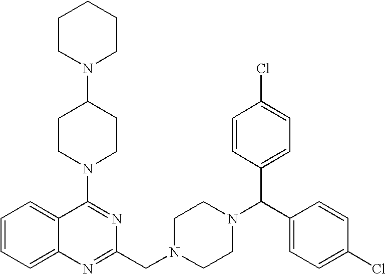 | | | | |
| 30-43 | 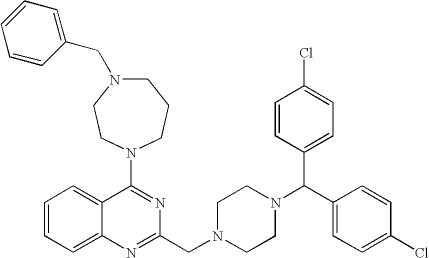 | | | | |
| 30-44 | 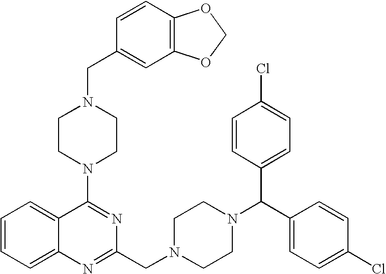 | | | | |
| 30-45 | 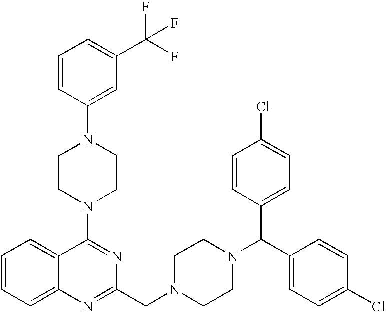 | | | | |
| 30-46 | 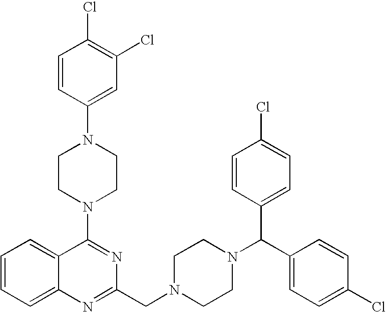 | | | | |
| 30-47 | 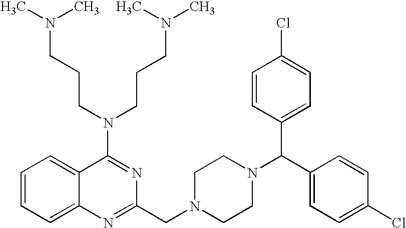 | | | | |
| 30-48 | 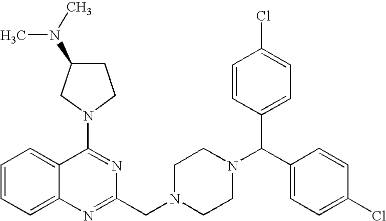 | | | | |
| 30-49 | 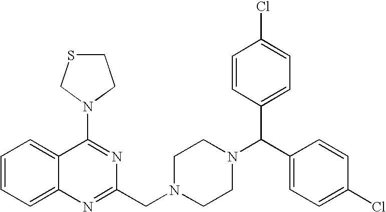 | | | | |
| 30-50 | 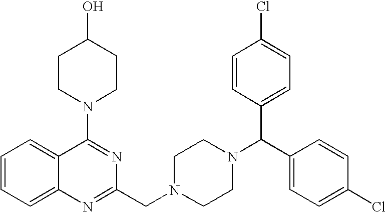 | | | | |
| 30-51 | 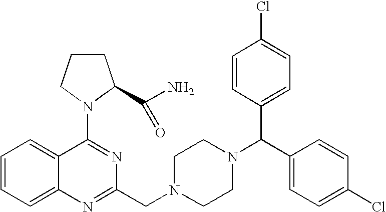 | | | | |
| 30-52 | 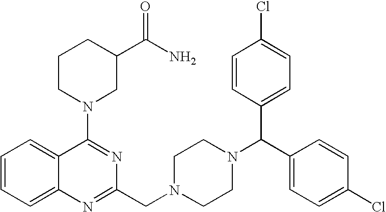 | | | | |
| 30-53 | 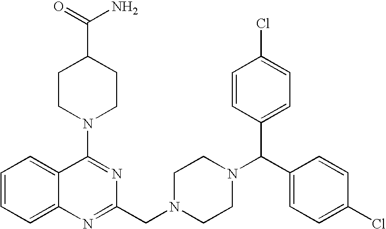 | | | | |
| 30-54 | 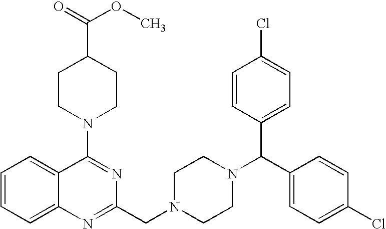 | | | | |
| 30-55 | 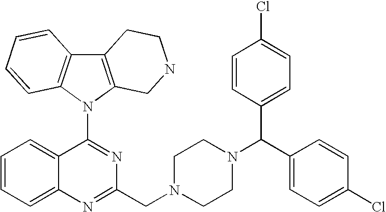 | | | | |
| 30-56 | 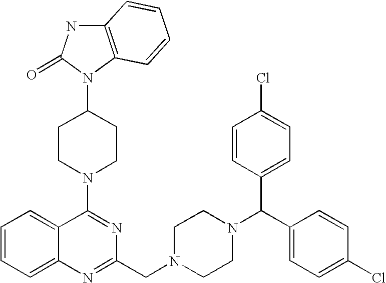 | | | | |
| 30-57 | 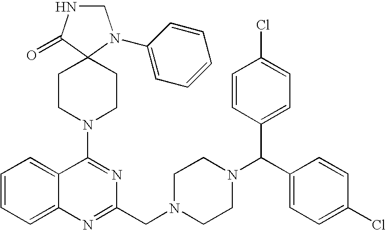 | | | | |
| 30-58 | 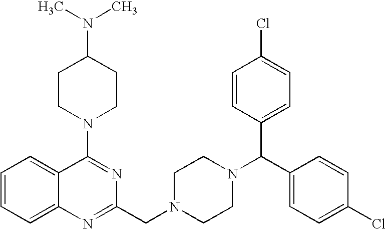 | | | | |

### Example 31

### Reaction of 2-{4-[BIS-(4-CHLOROPHENYL)METHYL]PIPERAZIN-1-YLMETHYL}-4-CHLOROQUINAZOLINE with an AMINO ALCOHOL LIBRARY

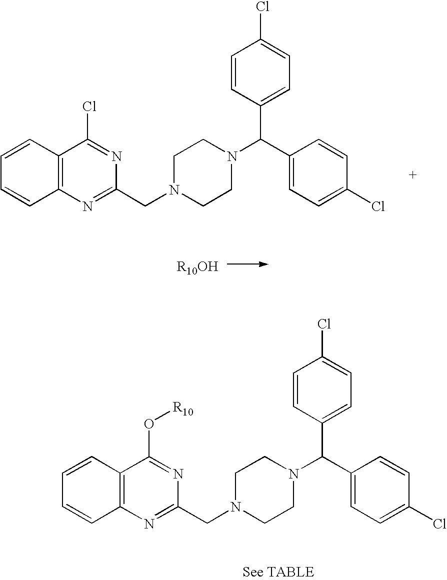

A stock solution of 2-{4-[bis-(4-chlorophenyl)methyl]piperazin-1-ylmethyl}-4-chloroquinazoline (50 mg, 0.1 mmoles) (prepared as described in Preparative Example 8 above) in anhydrous acetonitrile (5 mL) may be placed in each of the vessels in a Miniblock XT Solution Phase Synthesizer together with anhydrous potassium carbonate (0.1 mmoles). A 1M stock solution of each of the aminoalcohols (0.2 mmoles) in anhydrous acetonitrile is added and the Miniblock may be sealed and shaken at 80° C. for 24 h. The Miniblock is cooled to 25° C. and the contents of each vessel may be evaporated to dryness and chromatographed on silica gel using 0.5-5% (10% conc. ammonium hydroxide in methanol)-dichloromethane as the eluant to give the products listed in the table below.

### Examples 31-1 Through 31-6

| EXAMPLE | STRUCTURE | MW | LCMS m/z |
|---|---|---|---|
| 31-1 | 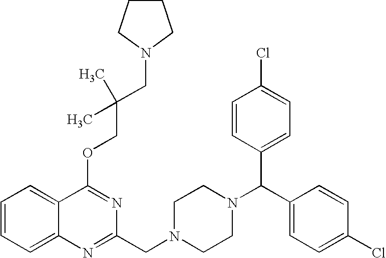 | 618.7 | |
| 31-2 | 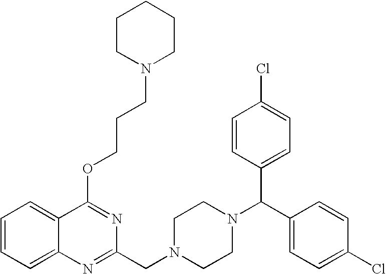 | 604.6 | |
| 31-3 | 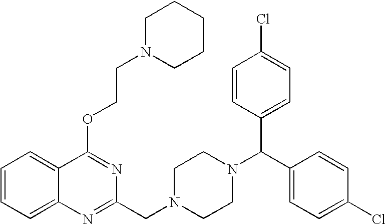 | 590.6 | |
| 31-4 | 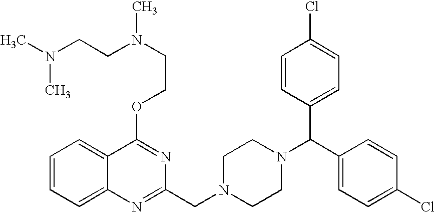 | 606.6 | |
| 31-5 | 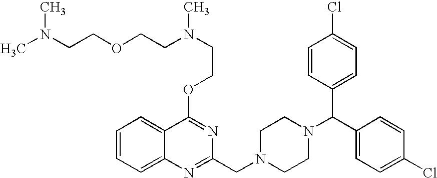 | 651.7 | |
| 31-6 | 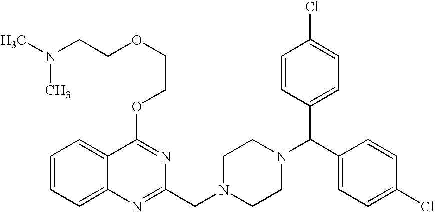 | 594.6 | |

### Example 43

### 2(S)-(−)-2-(2-{4-[BIS-(4-CHLOROPHENYL)METHYL]PIPERAZIN-1-YLMETHYL}QUINAZOLIN-4-YLAMINO)-4-DIMETHYLAMINOBUTYRAMIDE

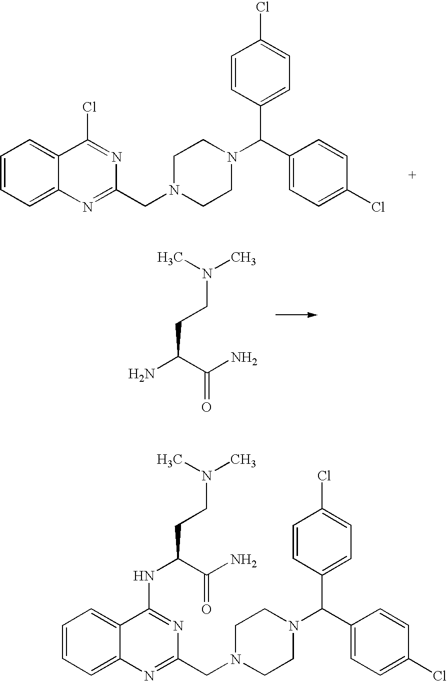

2-{4-[Bis-(4-chlorophenyl)methyl]piperazin-1-ylmethyl}-4-chloroquinazoline (62.5 mg, 0.1 mmoles) (prepared as described in Preparative Example 8 above) and 2(S)-amino-4-dimethylaminobutyramide (38 mg, 0.2 mmoles) (prepared as described in Preparative Example 35 above) were dissolved in 200 proof ethanol (6 mL) and the mixture was heated under nitrogen at 80° C. for 22 h. The solution was evaporated to dryness and the residue was chromatographed on a silica gel column (30×2.5 cm) using 5% (10% concentrated ammonium hydroxide in methanol)-dichloromethane as the eluant to give 2(S)-(−)-2-(2-{4-[bis-(4-chlorophenyl)methyl]piperazin -1-ylmethyl}quinazoline-4-ylamino)-4-dimethylaminobutyramide (46.3 mg, 58%): ESMS: m/z 607.86 (MH⁺); HRFABMS: m/z 606.2499 (MH⁺). Calcd. for C₃₂H₃₈Cl₂N₇O: m/z 606.2515; δ_{H} (CDCl₃) 2.02 (2H, m, CHCH₂CH₂N(CH₃)₂), 2.27 (1H, m, CHCH₂CH₂N(CH₃)₂), 2.43 (6H, s, N(CH₃)₂), 2.48 (4H, m, N(CH₂CH₂)N), 2.57 (1H, m, CHCH₂CH₂N(CH₃)₂), 2.70 (4H, m, N(CH₂CH₂)N), 2.97 (1H, m, NH), 3.68/3.80 (2H, AB system, 2-CH₂N), 4.22 (1H, s, NCH(C₆H₄Cl)₂), 4.92 (1H, m, CHCH₂CH₂N(CH₃)₂), 5.45 (1H, bs, CONH₂), 7.30 (4H, m, NCH(C₆H₄Cl)₂), 7.36 (4H, m, NCH(C₆H₄Cl)₂), 7.48 (1H, m, H₆), 7.68 (1H, m, H₅), 7.74 (1H, m, H₇), 7.86 (1H, m, H₈) and 9.48 ppm (1H, bs, CONH₂); δ_{C} (CDCl₃) CH₃: 45.7, 45.7; CH₂: 26.9, 52.3, 52.3, 54.1, 54.1, 57.7, 65.8; CH: 55.3, 75.2, 121.6, 126.2, 128.8, 129.5, 129.5, 129.5, 129.5, 129.6, 129.6, 129.6, 129.6, 133.0; C: 114.5, 133.2, 133.2, 141.3, 141.3, 150.3, 160.0, 163.5, 174.9; [α]_{D}^{25° C. -}2.68° (c=0.50, MeOH). The compound was found to have % Residual T @ 2 ug/mL rating according to scintillation proximity assay (SPA) of “C”

### Example 44

### 2(S)-(+)-2-(2({4-[BIS-(4-CHLOROPHENYL)METHYL]PIPERAZIN-1-YLMETHYL}QUINAZOLIN-4-YLAMINO)-4-DIMETHYLAMINOBUTYRIC ACID ETHYL ESTER

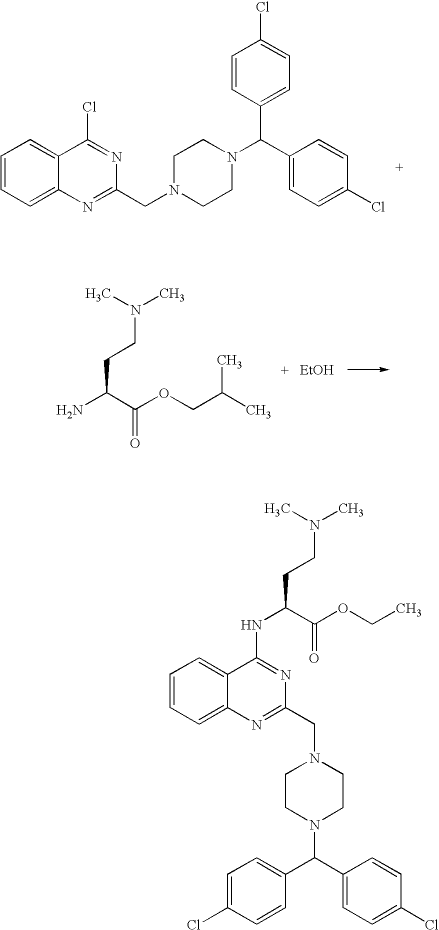

2-{4-[Bis-(4-chlorophenyl)methyl]piperazin-1-ylmethyl}-4-chloroquinazoline (174.7 mg, 0.4 mmoles) (prepared as described in Preparative Example 8 above) and 2(S)-(+)-amino-4-dimethylaminobutyric acid isobutyl ester (142 mg, 0.8 mmoles) (prepared as described in Preparative Example 31 above) were dissolved in 200 proof ethanol (6 mL) and the mixture was heated under nitrogen at 80° C. for 40 h. The solution was evaporated to dryness and the residue was chromatographed on a silica gel column (30×2.5 cm) using 3% (10% concentrated ammonium hydroxide in methanol)-dichloromethane as the eluant to give 2(S)-(+)-2-(2-{4-[bis-(4-chlorophenyl)methyl]piperazin-1-ylmethyl}quinazoline-4-ylamino)-4-dimethylaminobutyric acid ethyl ester (162 mg, 73%): ESMS: m/z 635.12 (MH⁺); HRFABMS: m/z 635.2679 (MH⁺). Calcd. for C₃₄H₄₁Cl₂N₆O₂: m/z 635.2668; δ_{H} (CDCl₃) 1.28 (3H, t, COOCH₂CH₃), 2.05 (2H, m, CHCH₂CH₂N(CH₃)₂), 2.22 (2H, m, CHCH₂CH₂N(CH₃)₂), 2.42 (6H, s, N(CH₃)₂), 2.48 (4H, m, N(CH₂CH₂)N), 2.72 (4H, m N(CH₂CH₂)N), 2.77 (1H, m, NH), 3.77 (1H, s, NCH(C₆H₄Cl)₂), 4.18/4.27 (2H, AB system, 2-CH₂N), 4.91 (1H, m, CHCH₂CH₂N(CH₃)₂), 7.27 (4H, m, NCH(C₆H₄Cl)₂), 7.37 (4H, m, NCH(C₆H₄Cl)₂), 7.48 (1H, m, H₆), 7.67 (1H, m, H₇), 7.72 (1H, m, H₅), 7.87 (1H, m, H₈) and 9.41 ppm (1H, bs, CONH₂); δ_{C} (CDCl₃) CH₃: 14.7, 45.9, 45.9; CH₂: 27.4, 52.2, 52.2, 53.8, 53.8, 57.4, 65.6; CH: 55.5, 75.5, 121.6, 125.6, 128.7, 129.1, 129.1, 129.1, 129.1, 129.6, 129.6, 129.6, 129.6, 132.7; C: 114.3, 133.1, 133.1, 141.4, 141.5, 150.4, 159.8, 163.3, 173.1; [α]_{D}^{25° C.} +2.77° (c=0.97, MeOH). The compound was found to have % Residual T @ 2 ug/mL rating according to scintillation proximity assay (SPA) of “C”

### Example 45

### 2(S)-(−)-2-(2-{4-[BIS-(4-CHLOROPHENYL)METHYL]PIPERAZIN-1-YLMETHYL}QUINAZOLIN-4-YLAMINO)-5-DIMETHYLAMINOPENTANOIC ACID ISOBUTYL ESTER

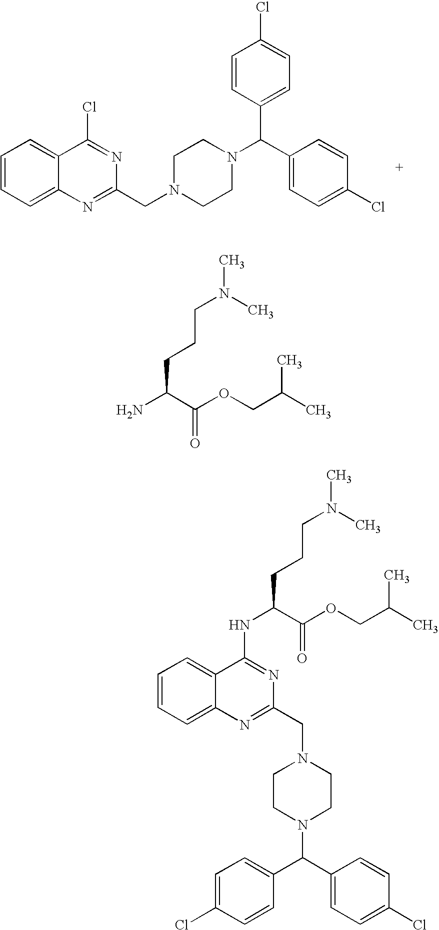

2-{4-[Bis-(4-chlorophenyl)methyl]piperazin-1-ylmethyl}-4-chloroquinazoline (141.5 mg, 0.3 mmoles) (prepared as described in Preparative Example 8 above) and 2(S)-(+)-amino-5-dimethylaminopentanoic acid isobutyl ester (123 mg, 0.6 mmoles) (prepared as described in Preparative Example 38 above) were dissolved in 200 proof ethanol (6 mL) and the mixture was heated under nitrogen at 80° C. for 22 h. The solution was evaporated to dryness and the residue was chromatographed on a silica gel column (30×2.5 cm) using 4% (10% concentrated ammonium hydroxide in methanol)-dichloromethane as the eluant to give 2(S)-(−)-2-(2-{4-[bis-(4-chlorophenyl)methyl]piperazin-1-ylmethyl}quinazoline-4-ylamino)-5-dimethylaminopentanoic acid isobutyl ester (113.2 mg, 59%): ESMS: m/z 677.14 (MH⁺); HRFABMS: m/z 677.3142 (MH⁺). Calcd. for C₃₇H₄₇Cl₂N₆O₂: m/z 677.3138; δ_{H} (CDCl₃) 0.91 (6H, d, COOCH₂CH(CH₃)₃), 1.72 (2H, m, CHCH₂CH₂CH₂N(CH₃)₂), 1.94 (1H, m, CHCH₂CH₂CH₂N(CH₃)₂), 2.08 (1H, m, CHCH₂CH₂CH₂N(CH₃)₂), 2.20 (1H, m, CHCH₂CH₂CH₂N(CH₃)₂), 2.20 (1H, m, COOCH₂CH(CH₃)₂), 2.32 (6H, s, N(CH₃)₂), 2.42 (1H, m, CHCH₂CH₂CH₂N(CH₃)₂), 2.48 (4H, m, N(CH₂CH₂)N), 2.76 (4H, m N(CH₂CH₂)N), 3.74 (2H, s, COOCH₂CH(CH₃)₂), 3.88/3.95 (2H, AB system, 2-CH₂N), 4.23 (1H, m, NH), 4.88 (1H, m, CHCH₂CH₂CH₂N(CH₃)₂), 7.28 (4H, m, NCH(C₆H₄Cl)₂), 7.37 (4H, m, NCH(C₆H₄Cl)₂), 7.47 (1H, m, H₆), 7.72 (1H, m, H₇) and 7.88 ppm (2H, m, H₅ and H₈); δ_{C} (CDCl₃) CH₃: 19.5, 19.5, 46.0, 46.0; CH₂: 23.9, 30.6, 52.2, 52.2, 53.9, 53.9, 60.1, 65.7, 71.6; CH: 28.2, 54.4, 75.2, 121.7, 125.6, 128.8, 129.1, 129.1, 129.1, 129.1, 129.6, 129.6, 129.6, 129.6, 132.7; C: 114.1, 133.1, 133.1, 141.4, 141.5, 150.5, 159.8, 163.3, 173.5; [α]_{D}^{25° C.} −11.48° (c=0.95, MeOH). The compound was found to have % Residual T @ 2 ug/mL rating according to scintillation proximity assay (SPA) of “D”. (See descriptions of assays below).

### Example 46

### 2(S)-(+)-2-(2-{4-[BIS-(4-CHLOROPHENYL)METHYL]PIPERAZIN-1-YLMETHYL}QUINAZOLIN-4-YLAMINO)-5-DIMETHYLAMINOPENTANAMIDE

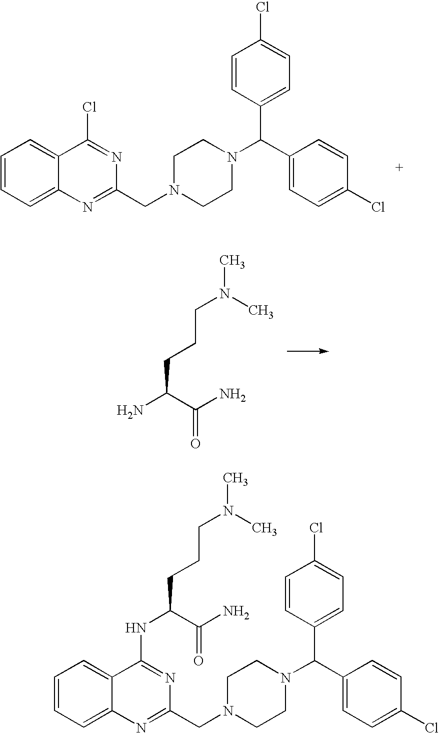

2-{4-[Bis-(4-chlorophenyl)methyl]piperazin-1-ylmethyl}-4-chloroquinazoline (234.5 mg, 0.5 mmoles) (prepared as described in Preparative Example 8 above) and 2(S)-(+)-amino-5-dimethylaminopentanamide (150 mg, 1.0 mmoles) (prepared as described in Preparative Example 42 above) were dissolved in 200 proof ethanol (6 mL) and the mixture was heated under nitrogen at 80° C. for 22 h. The solution was evaporated to dryness and the residue was chromatographed on a silica gel column (30×2.5 cm) using 4%-6% (10% concentrated ammonium hydroxide in methanol)-dichloromethane as the eluant to give 2(S)-(+)-2-(2-{4-[bis-(4-chlorophenyl)methyl]piperazin-1-ylmethyl}quinazoline-4-ylamino)-5-dimethylaminopentanamide (134.4 mg, 72%): ESMS: m/z 620.08 (MH⁺); HRFABMS: m/z 620.2678 (MH⁺). Calcd. for C₃₃H₄₀Cl₂N₇O: m/z 620.2671; δ_{H} (CDCl₃) 1.72 (1H, m, CHCH₂CH₂CH₂N(CH₃)₂), 1.89 (1H, m, CHCH₂CH₂CH₂N(CH₃)₂), 2.11 (2H, m, CHCH₂CH₂CH₂N(CH₃)₂), 2.30 (6H, s, N(CH₃)₂), 2.38 (2H, m, CHCH₂CH₂CH₂N(CH₃)₂), 2.47 (4H, m, N(CH₂CH₂)N), 2.70 (4H, m N(CH₂CH₂)N), 3.70/3.80 (2H, AB system, 2-CH₂N), 4.23 (1H, s, NCH(C₆H₄Cl)₂), 4.87 (1H, m, CHCH₂CH₂CH₂N(CH₃)₂), 5.57 (1H, m, NH), 7.28 (4H, m, NCH(C₆H₄Cl)₂), 7.34 (4H, m, NCH(C₆H₄Cl)₂), 7.48 (1H, m, H₆), 7.74 (1H, m, H₇) and 7.87 ppm (2H, m, H₅ and H₈); δ_{C} (CDCl₃) CH₃: 45.9, 45.9; CH₂: 24.0, 30.7, 52.2, 52.2, 54.1, 54.1, 60.0, 65.8; CH: 54.4, 75.2, 121.8, 126.0, 128.8, 129.2, 129.2, 129.2, 129.2, 129.5, 129.5, 129.5, 129.5, 133.1; C: 114.2, 133.2, 133.2, 141.3, 141.3, 150.4, 160.0, 163.3, 175.3; [α]_{D}^{25° C.} +3.23° (c=0.96, MeOH). The compound was found to have % Residual T @ 2 ug/mL rating according to scintillation proximity assay (SPA) of “D”. (See descriptions of assays below).

### Example 47

### 2(S)-(+)-2-(2-{4-[BIS-(4-CHLOROPHENYL)METHYL]PIPERAZIN-1-YLMETHYL}QUINAZOLIN-4-YLAMINO)-6-DIMETHYLAMINOHEXANAMIDE

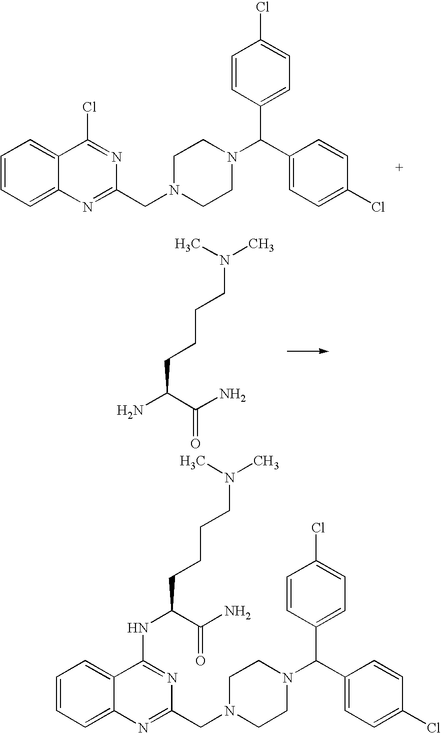

2-{4-[Bis-(4-chlorophenyl)methyl]piperazin-1-yl methyl}-4-chloroquinazoline (200 mg, 0.5 mmoles) (prepared as described in Preparative Example 8 above) and 2(S)-(+)-amino-6-dimethylaminohexanamide (139.2 mg, 1.0 mmoles) (prepared as described in Preparative Example 47 above) were dissolved in 200 proof ethanol (10 mL) and the mixture was heated under nitrogen at 80° C. for 40 h. The solution was evaporated to dryness and the residue was chromatographed on a silica gel column (30×2.5 cm) using 6%-20% (10% concentrated ammonium hydroxide in methanol)-dichloromethane as the eluant to give 2(S)-(+)-2-(2-{4-[bis-(4-chlorophenyl)methyl]piperazin-1-ylmethyl}quinazoline-4-ylamino)-6-dimethylaminohexanamide (146.4 mg, 57%): ESMS: m/z 634.09 (MH⁺); HRFABMS: m/z 634.2838 (MH⁺). Calcd. for C₃₄H₄₂Cl₂N₇O: m/z 634.2828; δ_{H} (CDCl₃) 1.53 (4H, m, CHCH₂CH₂CH₂CH₂N(CH₃)₂), 1.93 (1H, m, CHCH₂CH₂CH₂CH₂N(CH₃)₂), 2.08 (1H, m, CHCH₂CH₂CH₂CH₂N(CH₃)₂), 2.22 (6H, s, N(CH₃)₂), 2.29 (1H, m, CHCH₂CH₂CH₂CH₂N(CH₃)₂), 2.40 (1H, m, CHCH₂CH₂CH₂CH₂N(CH₃)₂), 2.47 (4H, m, N(CH₂CH₂)N), 2.68 (4H, m N(CH₂CH₂)N), 3.68/3.79 (2H, AB system, 2-CH₂N), 4.22 (1H, s, NCH(C₆H₄Cl)₂), 4.97 (1H, m, CHCH₂CH₂CH₂CH₂N(CH₃)₂), 5.73 (1H, m, CONH₂), 6.77 (1H, m, CONH₂), 7.28 (4H, m, NCH(C₆H₄Cl)₂), 7.36 (4H, m, NCH(C₆H₄Cl)₂), 7.43 (1H, m, H₆), 7.72 (1H, m, H₇), 7.84 (1H, m, H₅) and 7.88 ppm (1H, m, H₈); δ_{C} (CDCl₃) CH₃: 45.7, 45.7; CH₂: 23.5, 27.5, 31.6, 52.2, 52.2, 54.0, 54.0, 59.3, 65.9, 75.2; CH: 54.1, 121.5, 126.2, 128.7, 129.2, 129.2, 129.2, 129.2, 129.5, 129.5, 129.5, 129.5, 133.2; C: 114.0, 133.2, 133.2, 141.3, 141.3, 150.3, 159.6, 163.1, 175.1; [α]_{D}^{25° C.} +2.53° (c=1.09, MeOH). The compound was found to have % Residual T @ 2 ug/mL rating according to scintillation proximity assay (SPA) of “C”. (See descriptions of assays below).

### Proliferation Assay

This assay measures the growth suppression effects of small molecules in cells with mutant p53 vs. p53 null background. It uses Calcein AM to measure cellular viability. Cells (p53 null and mutant) are harvested and plated at 5000 cells per well in a 96-well tissue culture plate. The volume of cells in growth media is 100 □l. Serial dilutions (2× concentration) of compounds are then made and transferred to the plate of cells. The volume of compounds in the growth media is 100 μL. This dilution of compound with cells gives a 1× final dilution of compound (200 μL total volume). Plates are then incubated at 37° C. for 72 hours. Media is then poured off and Calcein AM is added at the appropriate concentration and the plates are incubated in the dark for 15 minutes and read for fluorescence. A letter rating corresponding to EC50 values (uM; MB468) from this assay have been assigned as follows: Compounds having EC50 values less than 2 uM have been assigned the letter “A”. Compounds having EC50 values of from 2 uM to less than 4 uM have been assigned the letter “B”. Compounds having EC50 values of from 4 uM to less than 6 uM have been assigned the letter “C”. Compounds having EC50 values of 6 uM or higher have been assigned the letter “D”. These letter rating has been used throughout in the data tables above. Specific EC50 values for some illustrative compounds are given in Table 2 below.

TABLE 2

| | | PROLIFERATION |
|---|---|---|
| COMPOUND | | ASSAY EC50 |
| # | STRUCTURE | MB468 (uM) |
| 1 | 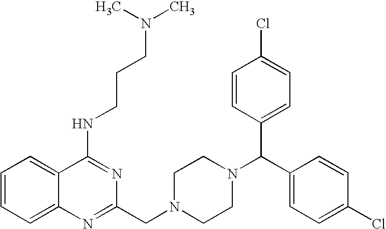 | 1.1 |
| 11-1 | 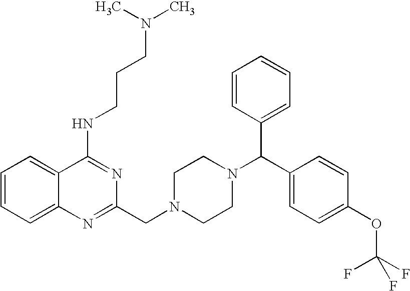 | 1.4 |
| 11-2 | 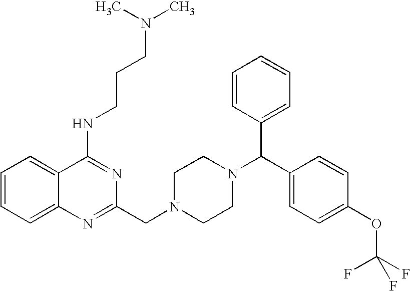 | 1.6 |
| 25-36 | 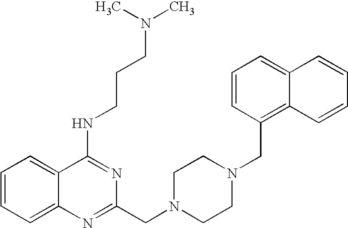 | 2.0 |
| 25-90 | 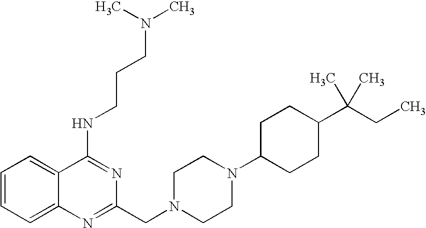 | 2.0 |
| Reference Structure CP-31398 (Pfizer) | 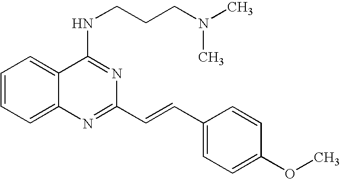 | 4.1 |

### Scintillation Proximity Assay (SPA)

Most of the oncogenic mutants of the tumor suppressor protein p53 lack sequence specific DNA binding activity at physiological temperature due to conformational changes in the DNA binding domain. Small molecules and peptide which bind to the p53 DNA binding domain stabilize the conformation and restore DNA binding activity to mutant p53 protein (Science 286, 2507-2510, 1999; PNAS, 99, 937-942, 2002). Using ³H STANDARD COMPOUND (which is ³H of the compound #1 of the present invention; the * Carbon atom is the one labeled; structure shown below), 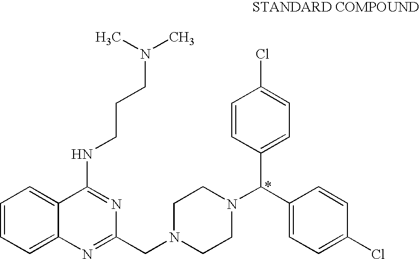
a radio labeled small molecule which binds to p53, and the GST-p53 DNA binding domain (aa 92-aa 312), we have developed a quantitative screening assay. The assay is based on Scintillation Proximity Assay (SPA) technology, developed by Amersham Biosciences to measure molecular interactions. Briefly, the complex of GST-p53, ³H STANDARD and Glutathione-SPA beads (Amersham Biosciences) are incubated with mixing for 1 hr at room temperature in the presence of the novel compounds to be screened. The signal is read on Microbeta. The compounds which have the ability to displace ³H STANDARD COMPOUND are selected. Such molecules will stabilize the conformation and restore DNA binding activity to mutant p53 protein.

The above assay was used to determine the ability of the compounds of this invention to restore DNA binding activity to mutant p53 and the results for selected compounds have been given above in various tables. Lower “% Residual total binding @ 2 ug/mL of drug” indicates superior performance.

A letter rating corresponding to % Residual total binding (T) @ 2 ug/mL of drug (i.e., compound of the present invention) from this assay have been assigned as follows: Compounds having % Residual T values of from 0% to less than 20% have been assigned the letter “A”. Compounds having % Residual T values of from 20% to less than 40% have been assigned the letter “B”. Compounds having % Residual T values of from 40% to less than 80% have been assigned the letter “C”. Compounds having % Residual T values of 80% or higher have been assigned the letter “D”. The exact % Residual T values @ 2 ug/mL for some illustrative compounds are shown below:

| | | SPA ASSAY |
|---|---|---|
| | | % Residual T @ |
| COMPOUND | | 2 ug/mL of drug |
| # | STRUCTURE | (Average of) |
| 1 | 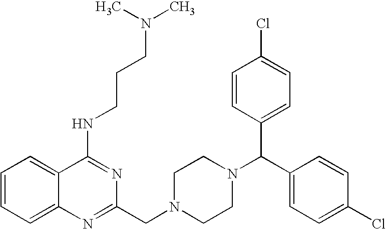 | 16.1 |
| 11-1 | 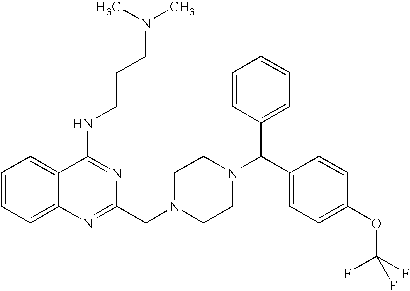 | 27.9 |
| | one enantiomer | |
| 20-29 | 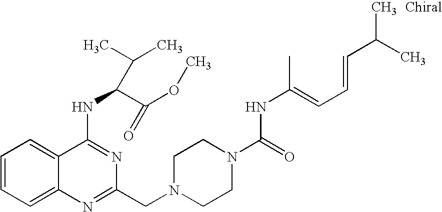 | 39.5 |
| 25-29 | 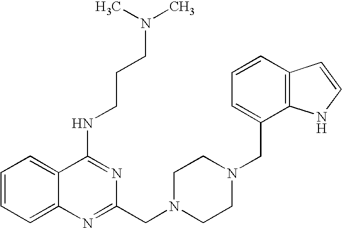 | 37.9 |
| 25-36 | 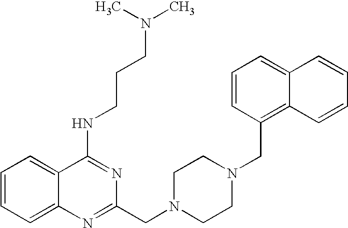 | 27.9 |
| 25-90 | 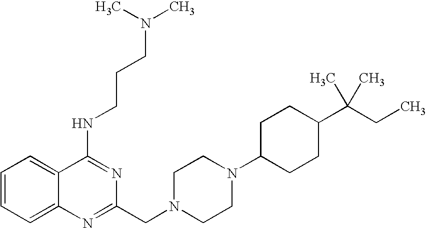 | 38.1 |
| 28-20 | 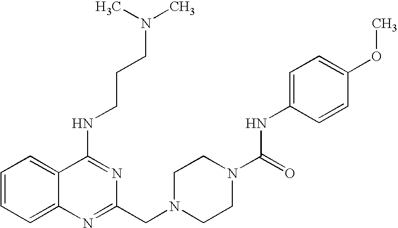 | 38.0 |
| 28-39 | 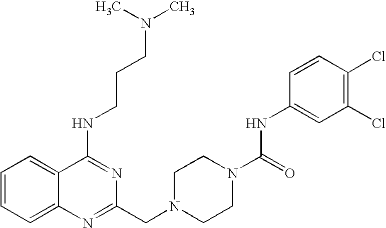 | 29.9 |

### Soft Agar Assay

This method assesses the ability of cells to grow in the absence of adhesion, which is a characteristic of tumorigenic cell lines. Small molecules are evaluated in this assay for their antitumor activity and the results are given in Table 3.

Human tumor DLD1 cells containing mutant p53 are suspended in growth medium containing 0.3% agarose and an indicated concentration of small molecule. The solution is overlayed onto growth medium solidified with 0.6% agarose containing the same concentration of the small molecule as the top layer. After the top layer is solidified the plates are incubated for 10-16 days at 37° C. under 5% CO₂ to allow colony outgrowth. After incubation, colonies are stained by overlaying the agar with a solution of MTT (3-[4,5-dimethyl-thiazol-2-yl]-2,5-diphenyltetrazolium bromide; Thiazolyl blue; 91 mg/mL in PBS). Colonies are counted to measure growth and efficacy of the small molecule.

TABLE 3

| COMPOUND # | SOFT AGAR IC₅₀ (μM) |
|---|---|
| 1 | 0.39 |

### In Vivo Antitumor Studies

Unstaged Model:

In this model the therapy was started immediately after the tumor cells had been inoculated.

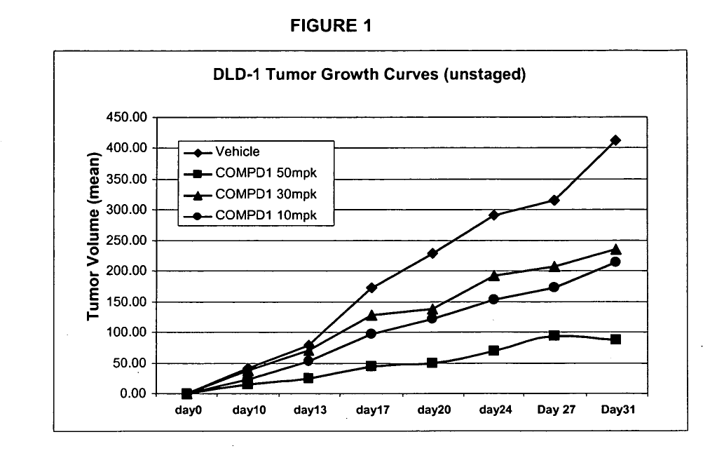

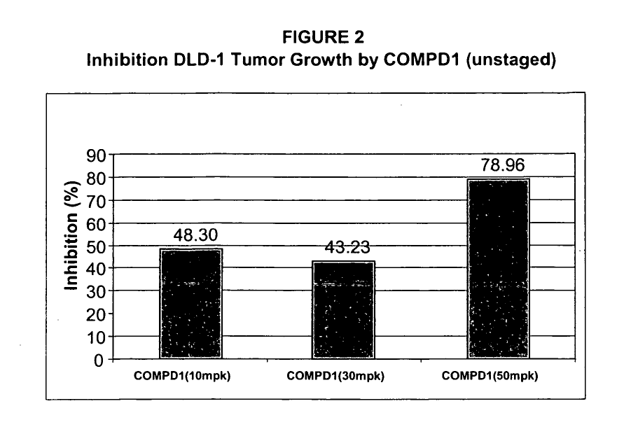

Nude mice, 5-6 week old female, were inoculated 5×10⁶ DLD-1 human colon adenocarcinoma cells on day 1, and randomized on day 3. The dosing of these mice was started on day 4. Groups 1 to 4, having 10 mice in each group, were dosed orally every 12 hour with vehicle, SCH 529074 10 mpk, SCH 529074 30 mpk, and SCH 529074 50 mpk, respectively, for 31 days. All animals were carefully monitored at least daily and each tumor was measured twice a week. Tumor growth curves (FIG. 1) and tumor growth inhibitions (FIG. 2) are shown below.

Staged Model:

In this model the initiation of therapy was delayed until the tumors had reached a certain volume.

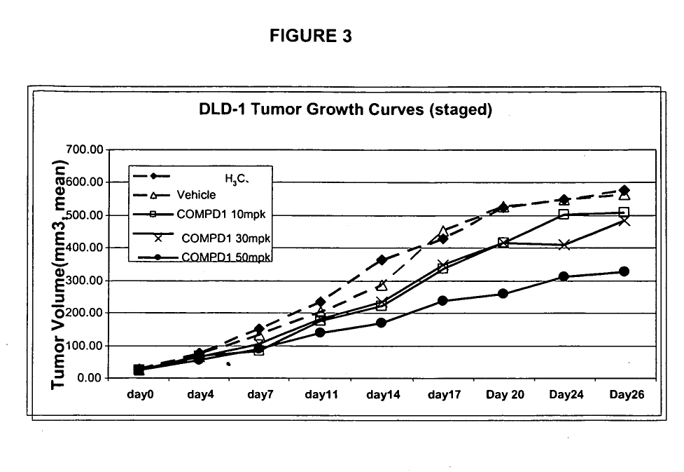

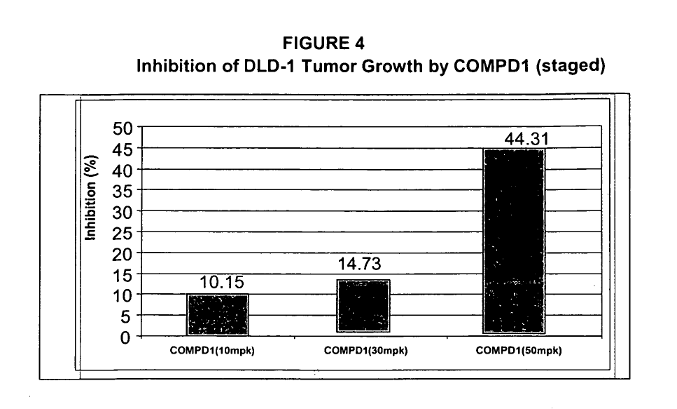

Nude mice, 5-6 week old female, were inoculated with 5×10⁶ DLD-1 human colon adenocarcinoma cells on day 1, and then randomized on day 10. The dosing of these mice was started on day 10. Groups 1 to 5, having 10 mice in each group, were dosed orally every 12 hour with no treatment, vehicle, SCH 529074 10 mpk, SCH 529074 30 mpk, and SCH 529074 50 mpk, respectively, for 26 days. All animals were carefully monitored at least daily and each tumor was measured twice a week. Tumor growth curves (FIG. 3) and tumor growth inhibitions (FIG. 4) are shown below.

Potentiation of Growth Suppression with Temozolomide

That the compounds of the present invention potentiate the growth suppression activity of temozolomide is illustrated by the fact that the compounds lower the temolozomide IC50 in various cell lines. The proliferation assay used is similar to that set forth above, and comprises the following general steps.
TMZ is diluted 2 fold in complete medium.
Cells are harvested and placed in each well with diluted TMZ. Cell conc. is 5000 cells per well in complete medium.
Appropriate concentration of compounds of present invention are then added to each well in combination with diluted TMZ.
Plates are incubated at 37° C. for 72 hrs.
Medium is poured off and 50 μL per well of Calcein AM (10 uM is added. Plate is then read in the fluorescent plate reader.

The results are shown in the Table below:

TABLE 4

| | | | | TMZ | TMZ IC50 (uM) | TMZ IC50 (uM) |
|---|---|---|---|---|---|---|
| | Tumor | p53 | MGMT | IC50 | @ 0.5 uM | @ 1.0 uM |
| Cell line | type | status | (fmol/mg) | (uM) | COMPD 25-36 | COMPD 25-36 |
| Capan-1 | Pancreas | mutant | 1200 | 2865 | 460 | 215 |
| Capan-2 | Pancreas | wild | 1680 | 4310 | 2065 | 1275 |
| Miapaca-2 | Pancreas | mutant | 900 | 3321 | 769 | 240 |
| U87 | Glioma | wild | 3 | 2542 | 364 | 256 |
| LN 18 | Glioma | mutant | | 2697 | 344 | 223 |

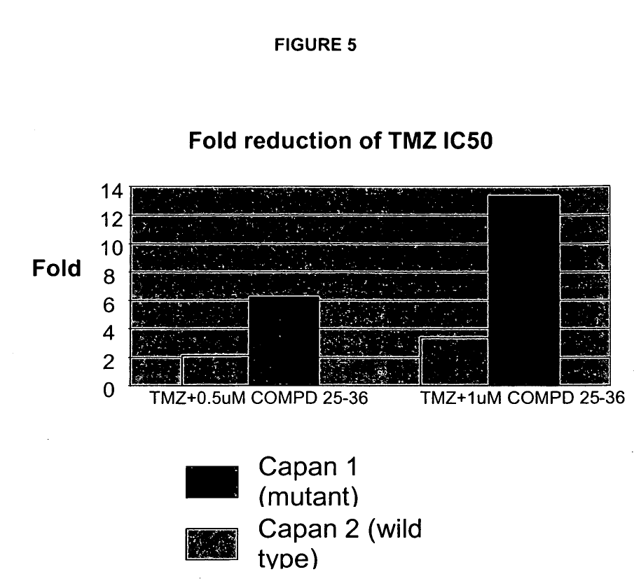

The data in the above table shows that the compounds of the present invention increases the sensitivity of pancreatic cells to temozolomide. The extent of increase can be quantitated as is shown below in FIG. 5.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A compound of formula I 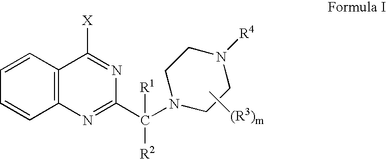
or a pharmaceutically acceptable salt, solvate or ester thereof, wherein:
(i) m is 0 to 2;
(ii) X is OR⁵ or N(R⁶)₂;
(iii) R¹ and R² are each independently selected from the group consisting of hydrogen and alkyl;
(iv) each R³ independently is alkyl;
(v) R⁴ is selected from the group of substituents consisting of alkyl, alkenyl, alkynyl, cycloalkyl, cycloalkenyl, aryl, heteroaryl, heterocyclyl, R⁷—(C═O)—, R⁸—(S(O)₂)—, and —(C═O)—NR⁹R¹⁰—, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when the aforesaid cycloalkyl and aryl substituents contains two moieties on the same carbon, such moieties may optionally be taken together with the carbon atom to which they are attached to form a carbocyclic or heterocyclic ring; wherein each of the aforesaid moieties containing an aryl alternative may optionally be independently substituted by one or two radicals independently selected from the group consisting of alkyl, halo, alkoxy, cyano, perhaloalkyl and perhaloalkoxy;
wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—;
wherein when said aryl moiety contains two radicals on adjacent carbon atoms anywhere within said moiety, such radicals may optionally and independently in each occurrence, be taken together with the carbon atoms to which they are attached to form a five to six membered carbocyclic or heterocyclyl ring; wherein each of the aforementioned radicals containing an aryl alternative may optionally be independently substituted by one or two radicals independently selected from the group consisting of alkyl, halo, alkoxy, cyano, perhaloalkyl and perhaloalkoxy;
(vi) R⁵, and each R⁶ are independently selected from the group consisting of hydrogen, alkyl, alkenyl, alkynyl, cycloalkyl, cycloalkenyl, aryl, heteroaryl, and heterocyclyl, wherein each of the R⁵ and R⁶ substituents alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, H₂N—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—NH—(C═O)—, alkyl-O—NH—(C═O)-alkyl-NH—(C═O)—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said cycloalkyl or aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring, which carbocyclic or heterocyclic ring may optionally be fused to an aryl ring;
wherein each of said aryl, cycloalkyl, heterocyclyl and heteroaryl moieties of said R⁵ and R⁶ substituents may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C≡O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy;
wherein when X is N(R⁶)₂, the two R⁶ groups may optionally be taken together with the nitrogen atom to which they are shown attached to form a heterocyclyl or heteroaryl ring which heterocyclyl or heteroaryl ring may optionally be independently substituted with one to two substituents independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;
wherein each of the aforesaid alkyl, alkenyl, aryl, arylalkyl-, cycloalkyl, heteroaryl, and heterocyclyl substituents may optionally be independently substituted with one to two moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl, heterocyclycl, heteroaryl and aryl moieties contains two radicals on adjacent carbon atoms, such radicals may optionally and independently in each occurrence, be taken together with the carbon atoms to which they are attached to form a five to six membered carbocyclic or heterocyclic ring; wherein when each of said cycloalkyl, heterocyclycl, heteroaryl and aryl moieties contains two radicals on the same carbon, such moieties may optionally be taken together with the carbon atom to which they are attached to form a five to six membered carbocyclic or heterocyclic ring; wherein each of the aforesaid moieties containing an aryl alternative may optionally be substituted by one or two radicals independently selected from the group consisting of alkyl, halo, alkoxy, cyano, perhaloalkyl and perhaloalkoxy;
(vii) R⁷ is selected from the group of substitutents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said R⁷ aryl or cycloalkyl substituent contains two moieties on adjacent carbon atoms, such moieties may optionally and independently in each occurrence, be taken together with the carbon atoms to which they are attached to form a five to six membered carbocyclic or heterocyclyl ring; wherein each of said moieties containing an aryl alternative may optionally be substituted by one or two radicals independently selected from the group consisting of alkyl, halo, alkoxy, cyano, perhaloalkyl and perhaloalkoxy;
(viii) R⁸ is selected from the group of substituents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said R⁸ aryl or cycloalkyl substituent contains two moieties on adjacent carbon atoms, such moieties may optionally and independently in each occurrence, be taken together with the carbon atoms to which they are attached to form a five to six membered carbocyclic or heterocyclyl ring, which carbocyclic or heterorcycli ring may optionally be substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy;
(ix) R⁹ is selected from the group of substituents consisting of alkyl, alkenyl, alkynyl, cycloalkyl, cycloalkenyl, aryl, heteroaryl, and heterocyclyl, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;
wherein each of said aryl, cycloalkyl, heterocyclyl, and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said radicals containing an aryl alternative may optionally be substituted by one or two groups independently selected from the group consisting of alkyl, halo, alkoxy, cyano, perhaloalkyl and perhaloalkoxy; and
(x) R¹⁰ is selected from the group consisting of hydrogen and alkyl;
with the following provisos:
(a) when X is OR⁵, R⁴ and R⁵ simultaneously are other than unsubstituted alkyl;
(b) when X is OR⁵, R⁴ is other than R⁸—(S(O)₂)—;
(c) when X is N(R⁶)₂ wherein each R⁶ is independently hydrogen or straight or branched alkyl with no further substitution, and R⁴ is R⁸—(S(O)₂)— wherein R⁸ is an aryl which may optionally be substituted, the substituents on said aryl are other than alkoxy and halo; and
(d) when X is N(R⁶)₂ wherein the two R⁶ groups are taken together with the nitrogen atom to which they are shown attached form a piperidine ring, R⁴ is other than R⁸—S(O)₂—.

2. The compound of claim 1, wherein X is N(R⁶)₂.

3. The compound of claim 2, wherein R¹ and R² are both hydrogen.

4. The compound of claim 2, wherein m is 0 or 1.

5. The compound of claim 4, wherein m is 0.

6. The compound of claim 4, wherein R⁴ is selected from the group of substituents consisting of alkyl and alkenyl;
wherein said R⁴ alkyl and alkenyl substituents may optionally be independently substituted by one to four moieties selected independently from the group consisting of alkyl-S—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy;
wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—;
wherein when said aryl moiety contains two radicals on adjacent carbon atoms anywhere within said moiety, such radicals may optionally and independently in each occurence be taken together with the carbon atoms to which they are attached to form a five to six membered carbocyclic or heterocyclic ring; wherein each of the aforementioned radicals containing an aryl alternative may optionally be independently substituted by one or two radicals independently selected from the group consisting of alkyl, halo, alkoxy, cyano, perhaloalkyl and perhaloalkoxy;

7. The compound of claim 6, wherein each of said R⁴ alkyl and alkenyl substituents may optionally be independently substituted by one to four moieties selected independently from the group consisting of aryl, cycloalkyl, heterocyclyl, heteroaryl, alkyl-S—, and fluorenyl;
wherein said aryl moiety may optionally be independently substituted by one to two radicals selected independently from the group consisting of alkyl, alkoxy, halo, hydroxyl, cyano, alkyl-S—, aryl-S-alkyl-S(O)₂—, alkyl-(C═O)—NH—, alkyl-O—(C═O)—, perhaloalkyl, aryl, aryloxy, aryl-alkynyl-, and alkyl-O—(C═O)-alkyl-O—; wherein when said aryl moiety contains two radicals on adjacent carbon atoms anywhere within said moiety, such radicals may optionally and independently in each occurrence, be taken together with the carbon atoms to which they are attached to form a five to six membered carbocyclic or heterocyclic ring; wherein each of said radicals containing an aryl alternative may optionally be substituted by one or two radicals independently selected from the group consisting of alkyl, halo, alkoxy, cyano, perhaloalkyl and perhaloalkoxy;
wherein said cycloalkyl moiety may optionally be independently substituted by one or two radicals selected independently from the group consisting of alkyl, halo, hydroxy, cyano, and alkyl-O—(C═O)—;
wherein said heterocyclyl moiety may optionally be independently substituted by one or two radicals selected independently from the group consting of halo, hydroxyl, alkoxy;
wherein said heteroaryl moiety may optionally be independently substituted by one or two radicals selected independently from the group consting of alkyl, hydroxyalkyl, heteroaryl, aryl, and aryl-S(O)₂—; wherein each of said radicals containing an aryl alternative may optionally be substituted by one or two radicals independently selected from the group consisting of alkyl, halo, alkoxy, cyano, perhaloalkyl and perhaloalkoxy.

8. The compound of claim 7, wherein said cycloalkyl moiety is selected from the group consisting of cyclopropyl, cyclobutyl, cyclopentyl, cyclohexyl and cycloheptyl, each of which may be optionally substituted.

9. The compound of claim 7, wherein said heterocyclyl moiety is selected from the group consisting of dihydropyranyl, tetrahydropyranyl, and piperidinyl, each of which may be optionally substituted.

10. The compound of claim 7, wherein said heteroaryl moiety is selected from the group consisting of pyridinyl, furanyl, thiophenyl, pyrrolyl, 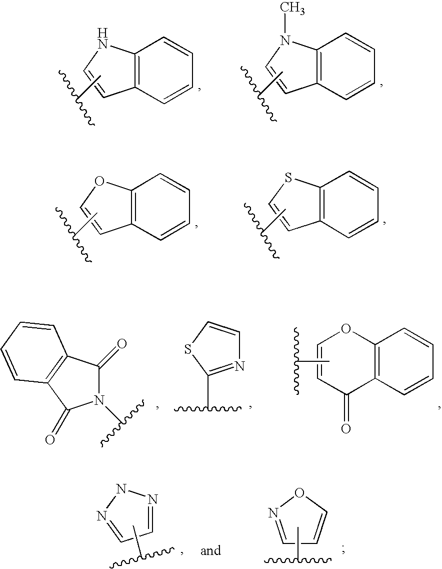
each of which may be optionally substituted.

11. The compound of claim 7, wherein said aryl moiety, including aryl moiety containing two radicals on adjacent carbon atoms which are taken together with the carbon atoms to which said radicals are attached to form a five to six memebered carbocyclic or heterocyclic ring, is selected from the group consisting of phenyl, naphthyl, 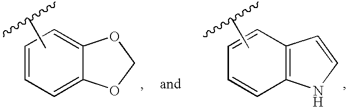
each of which may optionally be substituted.

12. The compound of claim 4, wherein R⁴ is selected from group of substituents consisting of cycloalkyl, cycloalkenyl and heterocyclyl;
wherein each of the aforesaid cycloalkyl, cycloalkenyl and heterocyclyl substituents may optionally be independently substituted by one to four moieties independently selected from the group consisting of halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of the aforesaid cycloalkyl, cycloalkenyl and heterocyclyl substituents contains two moieties on the same carbon, such moieties may optionally be taken together with the carbon atom to which they are attached to form a carbocyclic or heterocyclic ring; wherein when each of the aforesaid cycloalkyl, cycloalkenyl and heterocyclyl substituents contain two moieties on adjacent carbon atoms anywhere within said substituent, such moieties may optionally and independently in each occurrence, be taken together with the carbon atoms to which they are attached, to form a five to six membered carbocyclic or heterocyclic ring; wherein said aryl moiety and each of the aforesaid moieties containing an aryl alternative may optionally be independently substituted by one or two radicals independently selected from the group consisting of alkyl, halo, alkoxy, cyano, perhaloalkyl and perhaloalkoxy.

13. The compound of claim 12, wherein said R⁴ cycloalkyl, cycloalkenyl and heterocyclyl substituents may optionally be independently substituted by one to four moieties independently selected from the group consisting of cyano, alkyl, alkyl-(C═O)—, perhaloalkyl, aryl, and aryl-(C═O)—; wherein when each of the aforesaid cycloalkyl, cycloalkenyl and heterocyclyl substituents contains two moieties on the same carbon, such moieties may optionally be taken together with the carbon atom to which they are attached to form a carbocyclic or heterocyclic ring; wherein when each of the aforesaid cycloalkyl, cycloalkenyl and heterocyclyl substituents contains two moieties on adjacent carbon atoms anywhere within said substituent, such moieties may optionally and independently in each occurrence, be taken together with the carbon atoms to which they are attached, to form a five to six membered carbocyclic or heterocyclic ring; wherein the aryl and aryl-(C═O)— moieties may optionally be independently substituted by one or two radicals independently selected from the group consisting of alkyl, halo, alkoxy, cyano, perhaloalkyl and perhaloalkoxy.

14. The compound of claim 13, wherein said cycloalkyl substituent, including including cycloalkyl substituent containing two moieties on adjacent carbon atoms which are taken together with the carbon atoms to which said moieties are attached to form a five to six membered carbocyclic or heterocyclic ring, and including cycloalkyl substituent containing two moieties on the same carbon atom which are taken together with the carbon atom to which said moieties are attached to form a five to six membered carbocyclic or heterocyclic ring, is selected from the group consisting of multicyclic ring system, cyclopropyl, cyclobutyl, cyclopenyl, cyclohexyl, cycloheptyl, polycycloalkyl, 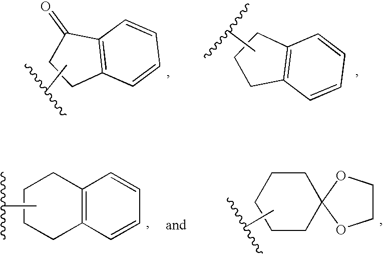
each of which may optionally be substituted.

15. The compound of claim 13, wherein said heterocyclyl substituent is selected from the group consisting of tetrahydrofuranyl, tetrahydropyranyl, tetrahydrothiophenyl, tetrahydrothiopyranyl, and piperidinyl, each of which may be optionally substituted.

16. The compound of claim 4, wherein R⁴ is R⁷—(C═O)—; wherein R⁷ is selected from the group of substitutents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said said R⁷ aryl, heteroaryl, heterocyclyl, and cycloalkyl substituents contains two moieties on adjacent carbon atoms anywhere within said substituent, such moieties may optionally and independently in each occurrence, be taken together with the carbon atoms to which they are attached to form a five to six membered carbocyclic or heterocyclyl ring; wherein each of said moieties containing an aryl alternative may optionally be substituted by one or two radicals independently selected from the group consisting of alkyl, halo, alkoxy, cyano, perhaloalkyl and perhaloalkoxy.

17. The compound of 16, wherein R⁷ is selected from the group of substitutents consisting of alkyl, alkenyl, aryl, cycloalkyl, heteroaryl, and heterocyclyl; wherein when each of said aryl, cycloalkyl, heteroaryl, and heterocyclyl substituents contains two moieties on adjacent carbon atoms anywhere within said substituent, such moieties may optionally and independently in each occurrence, be taken together with the carbon atoms to which such moieties are attached to form a five to six membered carbocyclic or heterocyclic ring;
wherein said alkyl and alkenyl substitutents may optionally be independently substituted with one to four moieties independently selected from the group consisting of alkyl, cycloalkyl, heterocyclyl, alkyl-S—, alkyl-O—(C═O)—, aryl, aryloxy, aryl-S—, and heteroaryl;
wherein said heterocyclyl substituent may optionally be substituted with one to four moieties independently selected from the group consisting of alkyl, halo, alkoxy, and alkyl-(C═O)—,
wherein said heteroaryl substitutent may optionally be substituted with one to four moieties independently selected from the group consisting of alkyl, aryl, halo and alkoxy;
wherein said aryl substitutent may optionally be substituted with one to four moieties independently selected from the group consisting of alkyl, alkyl-S—, cycloalkyl, alkoxy, halo, aryl, cyano, alkyl-(C═O)—NH—, and perhaloalkyl;
wherein said cycloalkyl substituent may optionally be substituted with one to four moieties independently selected from the group consisting of alkyl, halo, alkoxy, and aryl;
wherein said aryl moiety may optionally be substituted with one or two radicals selected from the group consisting of alkyl, cyano, halo, aryl, and perhaloalkyl;
wherein each of the aforesaid moieties containing an aryl alternative may optionally be substituted by one or two radicals independently selected from the group consisting of alkyl, halo, alkoxy, cyano, perhaloalkyl and perhaloalkoxy.

18. The compound of claim 17, wherein said cycloalkyl substituent, including cycloalkyl substituent containing two moieties on adjacent carbon atoms which are taken together with the carbon atoms to which said moieties are attached to form a five to six membered carbocyclic or heterocyclic ring, is selected from the group consisting of multicyclic ring system, cyclopropyl, cyclobutyl, cyclopenyl, cyclohexyl, cycloheptyl, polycycloalkyl, 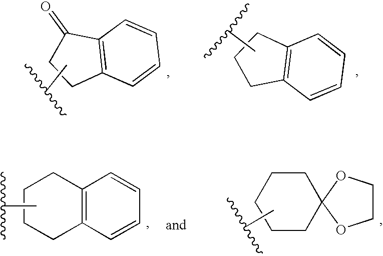
each of which may optionally be substituted.

19. The compound of claim 17, wherein said heteroaryl substituent, including heteroaryl substituent containing two moieties on adjacent carbon atoms which are taken together with the carbon atoms to which said moieties are attached to form a five to six membered carbocyclic or heterocyclic ring, is selected from the group consisting of pyridinyl, furanyl, thiophenyl, pyrrolyl, 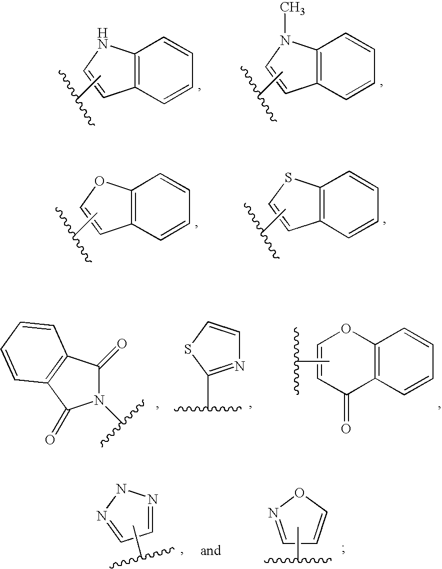
each of which may be optionally substituted.

20. The compound of claim 17, wherein said heterocyclyl substituent, including heterocyclyl substituent containing two moieties on adjacent carbon atoms which are taken together with the carbon atoms to which said moieties are attached to form a five to six membered carbocyclic or heterocyclic ring, is selected from the group consisting of tetrahydrofuranyl, tetrahydropyranyl, tetrahydrothiophenyl, tetrahydrothiopyranyl, piperidinyl, 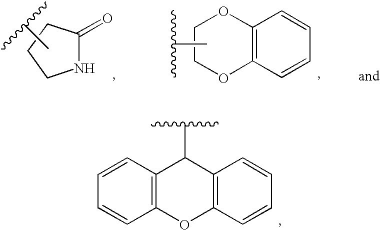
each of which may be optionally substituted.

21. The compound of claim 17, wherein said aryl substituent, including aryl substituent containing two moieties on adjacent carbon atoms which are taken together with the carbon atoms to which said radicals are attached, to form a five to six membered carbocyclic or heterocyclic ring, is selected from the group consisting of phenyl, naphthyl, 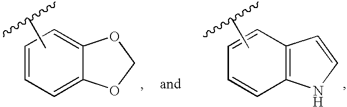
each of which may optionally be substituted.

22. The compound of claim 4, wherein R⁴ is R⁸—(S(O)₂)— wherein R⁸ is selected from the group of substituents consisting of alkyl, alkenyl, alkynyl, cycloalkyl, cycloalkenyl, aryl, heteroaryl, and heterocyclyl; wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said R⁸ aryl, heteroaryl, heterocyclyl, or cycloalkyl substituents contains two moieties on adjacent carbon atoms anywhere within said substituent, such moieties may optionally and independently in each occurrence, be taken together with the carbon atoms to which they are attached to form a five to six membered carbocyclic or heterocyclic ring, which carbocyclic or heterorcyclic ring may optionally be substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy.

23. The compound of claim 22, wherein R⁸ is selected from the group of substituents consisting of alkyl, alkenyl, heteroaryl, and aryl;
wherein said alkyl and alkenyl substitutents may optionally be independently substituted with one to four aryl moieties;
wherein said heteroaryl substitutent may optionally be substituted with one to four moieties independently selected from the group consisting of halo, alkyl, heteroaryl, alkyl-(C═O)—NH—, and alkyl-O—(C═O)—.
wherein said aryl substitutent may optionally be substituted with one to four moieties independently selected from the group consisting of alkyl, aryl, halo, cyano, alkoxy, alkyl-(C═O)—, alkyl-O—(C═O)—, alkyl-S(O)₂—, perhaloalkyl, perhaloalkoxy, and aryloxy; wherein when each of said aryl and heteroaryl substituents contains two moieties on adjacent carbon atoms anywhere within said substituent, such moieties may optionally and independently in each occurrence, be taken together with the carbon atoms to which they are attached to form a five to six membered carbocyclic or heterocyclic ring which carbocyclic or heterocyclic ring may optionally be independently substituted with one or two radicals selected independently from the group consisting of alkyl, alkyl-(C═O)—, perfluoroalkyl-(C═O)—, and halo; wherein each of said moieties containing an aryl alternative may optionally be substituted by one or two radicals independently selected from the group consisting of alkyl, halo, alkoxy, cyano, perhaloalkyl and perhaloalkoxy;
wherein said aryl moiety of said alkyl and alkenyl substituents may optionally be substituted with one or two radicals selected from the group consisting of alkyl, cyano, halo, aryl, and perhaloalkyl.

24. The compound of claim 23, wherein said heteroaryl substituent, including heteroaryl substituent containing two moieties on adjacent carbon atoms which are taken together with the carbon atoms to which said moieties are attached to form a five to six membered carbocyclic or heterocyclic ring, is selected from the group consisting of pyridinyl, furanyl, thiophenyl, pyrrolyl, 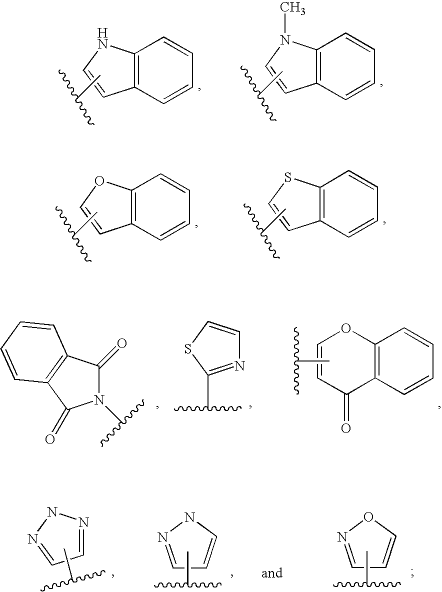
each of which may be optionally substituted.

25. The compound of claim 23, wherein said aryl substituent, including aryl substituent containing two moieties on adjacent carbon atoms which are taken together with the carbon atoms to which said moieties are attached to form a five to six membered carbocyclic or heterocyclic ring, is selected from the group consisting of phenyl, naphthyl, 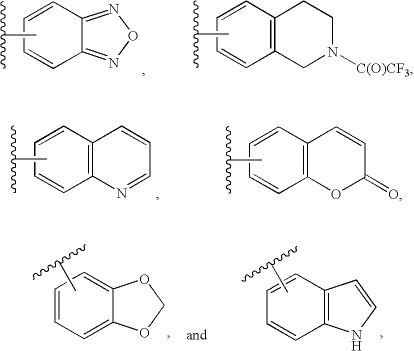
each of which may be optionally substituted.

26. The compound of claim 4, wherein R⁴ is —(C═O)—NR⁹R¹⁰—;
wherein R⁹ is selected from the group of substituents consisting of alkyl, alkenyl, alkynyl, cycloalkyl, cycloalkenyl, aryl, heteroaryl, and heterocyclyl, wherein each of the aforesaid alkyl, alkenyl, alkynyl, cycloalkyl, cycloalkenyl, aryl, heteroaryl and heterocyclyl substituents may optionally be independently substituted by one to four moieties independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;
wherein each of said aryl, cycloalkyl, heterocyclyl, and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said radicals containing an aryl alternative may optionally be substituted by one or two groups independently selected from the group consisting of alkyl, halo, alkoxy, cyano, perhaloalkyl and perhaloalkoxy; and
R¹⁰ is selected from the group consisting of hydrogen or alkyl.

27. The compound of claim 26, wherein R⁹ is selected from the group of substituents consisting of alkyl, cycloalkyl, and aryl;
wherein said alkyl substituent may optionally be substituted with one to four moieties independently selected from the group consisting of halo, alkoxy, hydroxyl, perhaloalkyl and aryl; wherein said aryl moiety may optionally be substituted with one or two radicals selected independently from the group consisting of alkyl, cyano, halo, aryl, and perhaloalkyl;
said cycloalkyl substitutent may optionally be substituted with one to four moieties independently selected from the group consisting of aryl, halo, alkyl, and alkoxy;
said aryl substitutent may optionally be substituted with one to four moieties independently selected from the group consisting of halo, alkyl, cyano, alkoxy, perhaloalkyl, nitro, and aryl; wherein when said aryl substituent contains two moieties on adjacent carbon atoms anywhere within said substituent, such moieties may optionally and independently in each occurrence, be taken together with the carbon atoms to which they are attached, to form a five to six membered carbocyclic or heterocyclic ring; and
R¹⁰ is selected from the group consisting of hydrogen or alkyl.

28. The compound of claim 27, wherein said cycloalkyl substituent is selected from the group consisting of cyclopropyl, cyclobutyl, cyclopenyl, cyclohexyl, and cycloheptyl, each of which may optionally be substituted.

29. The compound of claim 27, wherein said aryl substituent, including aryl substituent containing two moieties on adjacent carbon atoms which are taken together with the carbon atoms to which said moieties are attached to form a five to six membered carbocyclic or heterocyclic ring, is selected from the group consisting of phenyl, naphthyl, 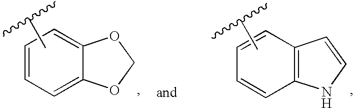
each of which may be optionally substituted.

30. The compound of claim 4, wherein one R⁶ is selected from the group of substituents consisting of hydrogen or alkyl, and the other R⁶ is selected from the group of substituents consisting of alkyl, cycloalkyl, heterocyclyl, heteroaryl and aryl; wherein each of the aforesaid other R⁶ alkyl, cycloalkyl, heterocyclyl, heteroaryl and aryl substituents may optionally be independently substituted by one to four moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl and aryl substituent contains two moieties on adjacent carbon atoms anywhere within said substituent, such moieties may optionally and independently in each occurence be taken together with the carbon atoms to which they are attached to form a five to six membered carbocyclic or heterocyclic ring;
wherein each of said aryl, cycloalkyl, heterocyclyl and heteroaryl moieties may optionally be independently substituted by one to two radicals selected independently from the group consisting of, methylenedioxy, alkyl-S—, aryl-S—, aryl-alkynyl-, alkyl-O—(C═O)-alkyl-O—, halo, alkyl, alkenyl, alkynyl, perhaloalkyl, aryl, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, fluorenyl, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein each of said moieties containing an aryl alternative may optionally be substituted by one or two radicals independently selected from the group consisting of alkyl, halo, alkoxy, cyano, perhaloalkyl and perhaloalkoxy.

31. The compound of claim 30, wherein one R⁶ is selected from the group of substituents consisting of hydrogen or alkyl, and the other R⁶ is selected from the group of substituents consisting of alkyl, cycloalkyl, heterocyclyl and aryl;
wherein the other R⁶ alkyl substituent is substituted by one to four moieties independently selected from the group consisting of amino, (alkyl)₂-amino, alkyl-O—(C═O)—, H₂N—(C═O)—, alkyl-O—NH—(C═O)—, alkyl-O—NH—(C═O)-alkyl-NH—(C═O)—, heterocyclyl, aryl, aryloxy and heteroaryl; wherein each of said heterocyclyl, aryl and aryloxy moieties may optionally be substituted by one or two radicals independently selected from the group consisting of alkyl, halo, cyano, alkoxy, perhaloalkyl and perhaloalkoxy; wherein when each of said aryl and aryloxy moieties contains two radicals on adjacent carbon atoms anywhere within said moiety, such radicals may optionally and independently in each occurence be taken together with the carbon atoms to which they are attached to form a five to six membered carbocyclic or heterocyclic ring;
wherein when the other R⁶ cycloalkyl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;
wherein the other R⁶ heterocyclyl substituent may optionally be substituted with arylalkyl-;
wherein the other R⁶ aryl substituent may optionally be substituted by one to four moieties independently selected from the group consisting of alkyl, alkoxy, halo, cyano, and alkyl-S—.

32. The compound of claim 31, wherein one R⁶ is hydrogen, and the other R⁶ is alkyl substituted by one or two moieties selected indendently from the group consisting of alkyl-(C═O)—, H₂N—(C═O)—, amino, and (alkyl)₂-amino.

33. The compound of claim 32, wherein N(R⁶)₂ is selected from the group consisting of

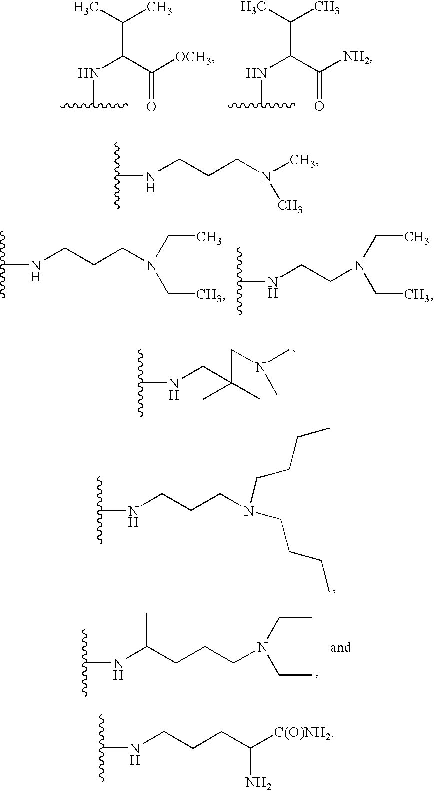

34. The compound of claim 31, wherein the heteroaryl moiety of the other R⁶ alkyl substituent is selected from the group consisting of imidazolyl, pyridinyl, furanyl, thiophenyl, and pyrrolyl, each of which may be optionally substituted.

35. The compound of claim 31, wherein the heterocyclyl moiety of the other R⁶ alkyl substituent is selected from the group consisting of tetrahydrofuranyl, tetrahydropyranyl, tetrahydrothiophenyl, tetrahydrothiopyranyl, piperidinyl, morpholinyl, piperizinyl, and pyrrolidinyl, each of which may be optionally substituted.

36. The compound of claim 31, wherein the aryl and aryloxy moieties of the other R⁶ alkyl substituent including aryl and aryloxy moieties containing two radicals on adjacent carbon atoms which are taken together with the carbon atoms to which said radicals are attached to form a five to six membered carbocyclic or heterocyclic ring, is selected from the group consisting of phenyl, phenyloxy, naphthyl, naphthyloxy, 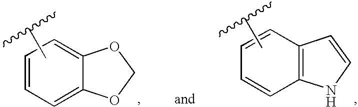
each of which may optionally be substituted.

37. The compound of claim 4, wherein the two R⁶ groups of N(R⁶)₂ are taken together with the nitrogen atom to which they are shown attached to form a heterocyclyl or heteroaryl ring which heterocyclyl or heteroaryl ring may optionally be independently substituted with one to two substituents independently selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)—, (alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when said aryl substituent contains two moieties on adjacent carbon atoms anywhere within said substituent, such moieties may optionally and independently in each occurrence, be taken together with the carbon atoms to which they are attached to form a five to six membered carbocyclic or heterocyclic ring;
wherein each of the aforesaid alkyl, alkenyl, aryl, arylalkyl-, cycloalkyl, heteroaryl, and heterocyclyl substituents may optionally be independently substituted with one to two moieties selected from the group consisting of halo, alkyl, alkenyl, alkynyl, perhaloalkyl, aryl, arylalkyl-, cycloalkyl, heteroaryl, heterocyclyl, formyl, —C≡N, alkyl-(C═O)—, aryl-(C═O)—, HO—(C═O)—, alkyl-O—(C═O)—, alkyl-NH—(C═O)-(alkyl)₂-N—(C═O)—, aryl-NH—(C═O)—, aryl-[(alkyl)-N]—(C═O)—, —NO₂, amino, alkylamino, (alkyl)₂-amino, alkyl-(C═O)—NH—, alkyl-(C═O)-[(alkyl)-N]—, aryl-(C═O)—NH—, aryl-(C═O)-[(alkyl)-N]—, H₂N—(C═O)—NH—, alkyl-HN—(C═O)—NH—, (alkyl)₂-N—(C═O)—NH—, alkyl-HN—(C═O)-[(alkyl)-N]—, (alkyl)₂-N—(C═O)-[(alkyl)-N]—, aryl-HN—(C═O)—NH—, (aryl)₂-N—(C═O)—NH—, aryl-HN—(C═O)-[(alkyl)-N]—, (aryl)₂-N—(C═O)-[(alkyl)-N]—, alkyl-O—(C═O)—NH—, alkyl-O—(C═O)-[(alkyl)-N]—, aryl-O—(C═O)—NH—, aryl-O—(C═O)-[(alkyl)-N]—, alkyl-S(O)₂NH—, aryl-S(O)₂NH—, alkyl-S(O)₂—, aryl-S(O)₂—, aryl-S—, hydroxy, alkoxy, perhaloalkoxy, aryloxy, alkyl-(C═O)—O—, aryl-(C═O)—O—, H₂N—(C═O)—O—, alkyl-HN—(C═O)—O—, (alkyl)₂-N—(C═O)—O—, aryl-HN—(C═O)—O— and (aryl)₂-N—(C═O)—O—; wherein when each of said cycloalkyl, heterocyclycl, heteroaryl and aryl moieties contains two radicals on adjacent carbon atoms anywhere within said moiety, such radicals may optionally and independently in each occurrence, be taken together with the carbon atoms to which they are attached to form a five to six membered carbocyclic or heterocyclic ring; wherein when each of said cycloalkyl, heterocyclycl, heteroaryl and aryl moieties contains two radicals on the same carbon, such radicals may optionally be taken together with the carbon atom to which they are attached to form a five to six membered carbocyclic or heterocyclic ring; wherein each of the aforesaid moieties containing an aryl alternative may optionally be substituted by one or two radicals independently selected from the group consisting of alkyl, halo, alkoxy, cyano, perhaloalkyl and perhaloalkoxy.

38. The compound of claim 37, wherein the two R⁶ groups of N(R⁶)₂ are taken together with the nitrogen atom to which they are shown attached form a heterocyclyl or heteroaryl ring which heterocyclyl or heteroaryl ring may optionally be independently subsitiuted with one to two substituents independently selected from the group consisting of halo, alkyl, hydroxy, cycloalkyl, aryl, heteroaryl, heterocyclyl, aryl-(C═O)—, heterocyclyl-(C═O)—, heteroaryl-(C═O)—, arylalkyl-O—(C═O)—, (alkyl)₂-N—(C═O)—, (alkyl)₂-amino, H₂N—(C═O)—, alkyl-O—(C═O)—, and alkyl-(C═O)—; wherein when said heterocyclyl or heteroaryl ring formed by said N(R⁶)₂ contains two moieties on adjacent carbon atoms anywhere within said heterocyclyl or heteroaryl ring, such moieties may optionally and independently in each occurrence, be taken together with the carbon atoms to which they are attached to form a five to six membered carbocyclic or heterocyclic ring;
wherein when each of said cycloalkyl, aryl, heteroaryl, and heterocyclyl substituents of said heterocyclyl or heteroaryl ring contains two moieties on adjacent carbon atoms anywhere within said substituent, such moieties may optionally and independently in each occurrence, be taken together with the carbon atoms to which they are attached, to form a five to six membered carbocyclic or heterocyclic ring;
wherein the alkyl substituent may optionally be substituted with one to two moieties independently selected from the group consisting of alkoxy, halo, and aryl moieties wherein each of said aryl moieties may optionally be substituted with one to two radicals independently selected from the group consisting of alkyl, cyano, halo, perhaloalkyl, and perhaloalkoxy; wherein when said aryl moiety contains two radicals on adjacent carbon atoms anywhere within said aryl moiety, such radicals may optionally and independently in each occurrence, be taken together with the carbon atoms to which they are attached to form a five to six membered carbocyclic or heterocyclyl ring;
wherein the aryl substituent may optionally be substituted with one or two moieties independently selected from the group consisting of halo, and perhaloalkyl;
wherein when the heterocyclyl substituent contains two moieties on the same carbon atom, such moieties may optionally be taken together with the carbon atom to which they are attached to form a five or six membered carbocyclic or heterocyclic ring.

39. The compound of claim 38, wherein the two R groups of N(R⁶)₂ are taken together with the nitrogen atom to which they are shown attached form a heterocyclyl ring, wherein said heterocyclyl ring, including heterocyclyl ring containing two moieties on adjacent carbon atoms which are taken together with the carbon atoms to which they are attached to form a five to six membered carbocyclic or heterocyclyl ring, is selected from the group consisting of pyrrolidinyl, morpholinyl, hexamethyleneiminyl, piperizinyl, piperidinyl, thiomorpholinyl, azacyclopropyl, homopiperizinyl, thiazolidinyl, 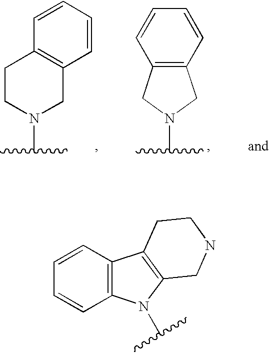
and each of which may be optionally substituted.

40. The compound of claim 1, selected from the group consisting of

| COMPOUND | | COMPOUND | |
|---|---|---|---|
| # | STRUCTURE | # | STRUCTURE |
| 1 | 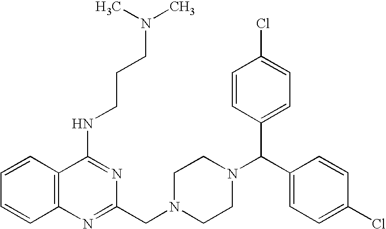 | 4-3 | 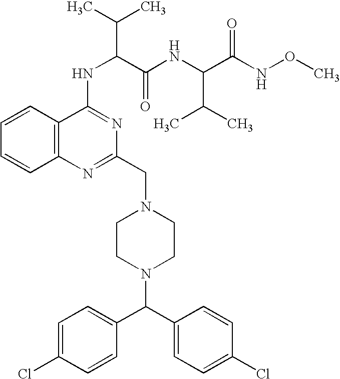 (+)-isomer |
| 2 | 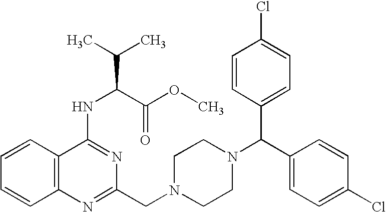 | 5-1 | 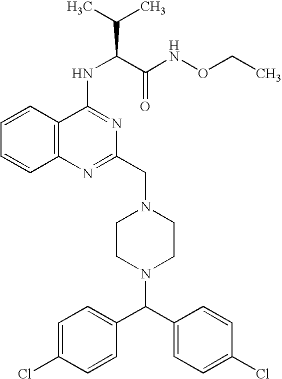 |
| 3 | 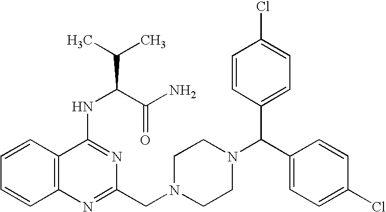 | 5-2 | 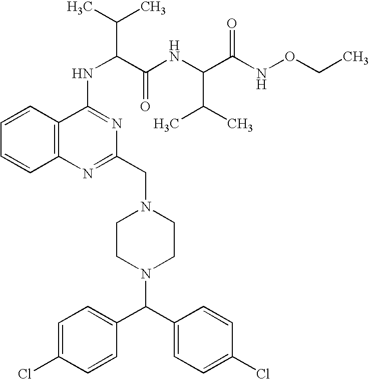 (−)-isomer |
| 4-1 | 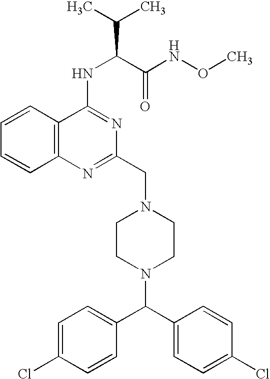 | 5-3 | 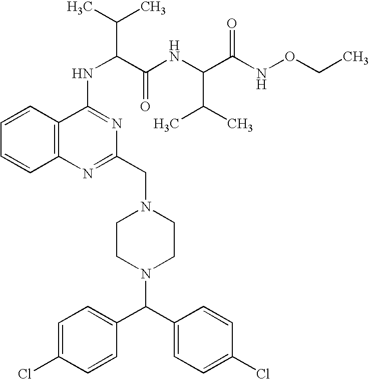 (−)-isomer |
| 4-2 | 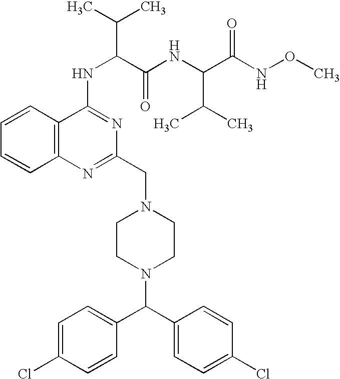 (−)-isomer | 6 | 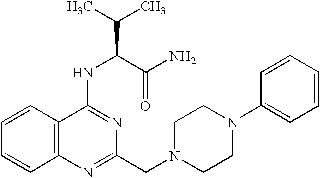 |
| 7 | 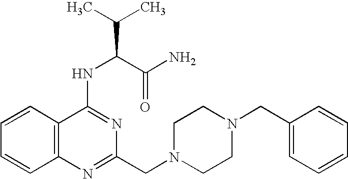 | 12-1 | 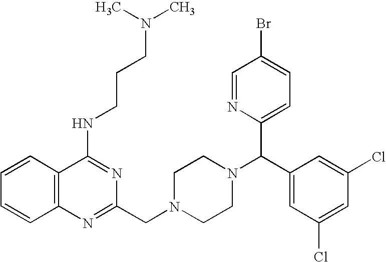 (+)-isomer |
| 8 | 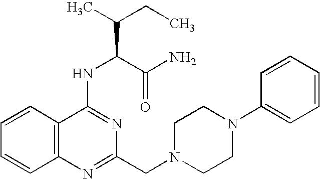 | 12-2 | 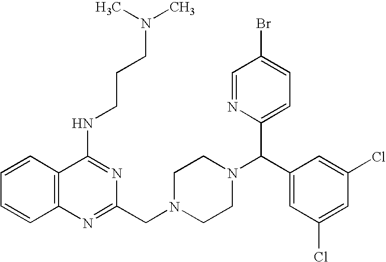 (−)-isomer |
| 9 | 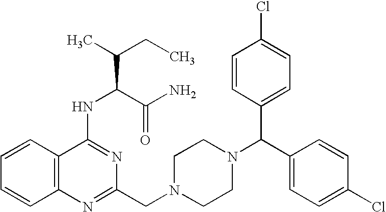 | 13-1 | 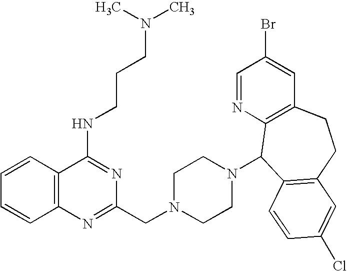 (+)-isomer |
| 10 | 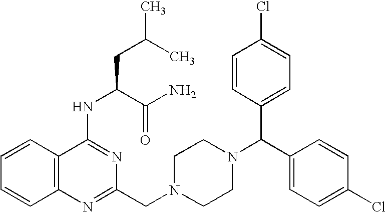 | 13-2 | 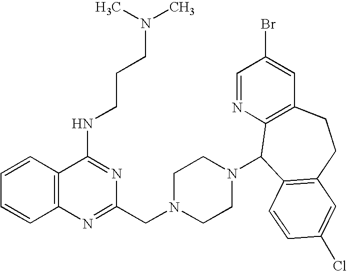 (−)-isomer |
| 11-1 | 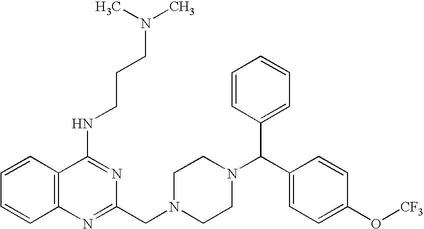 (+)-isomer | 14 | 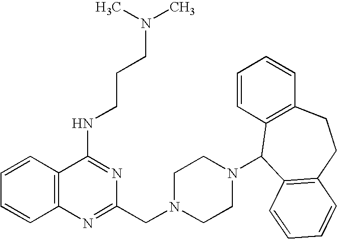 |
| 11-2 | 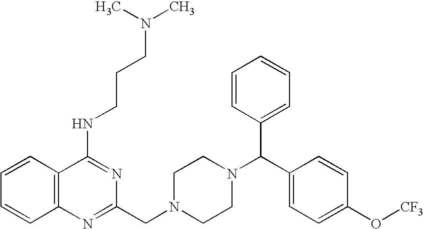 (−)-isomer | 15 | 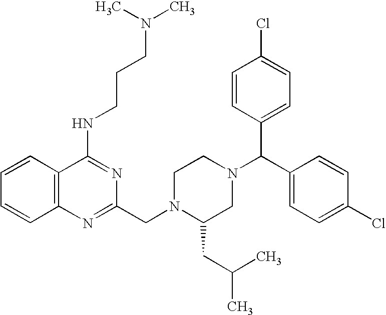 |
| 17-1 | 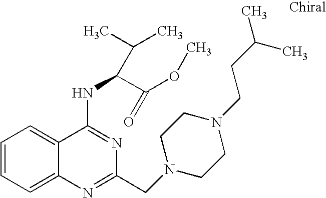 | 17-8 | 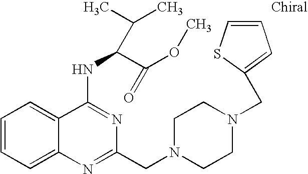 |
| 17-2 | 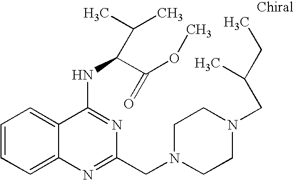 | 17-9 | 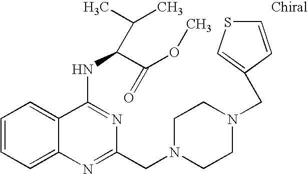 |
| 17-3 | 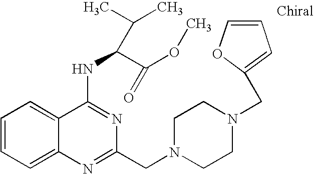 | 17-10 | 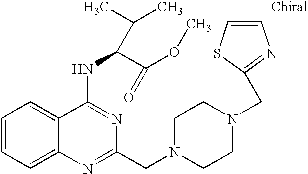 |
| 17-4 | 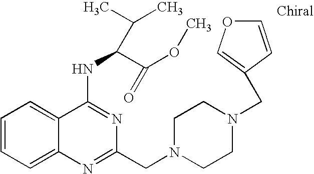 | 17-11 | 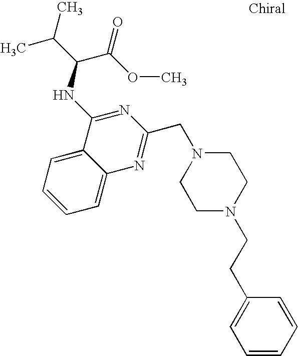 |
| 17-5 | 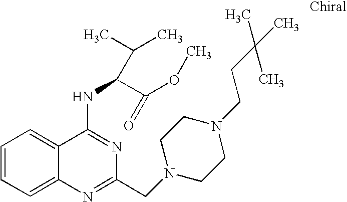 | 17-12 | 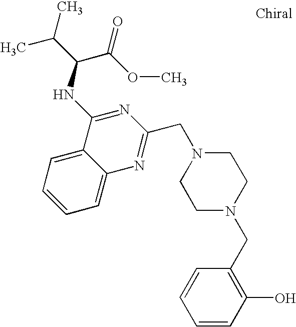 |
| 17-6 | 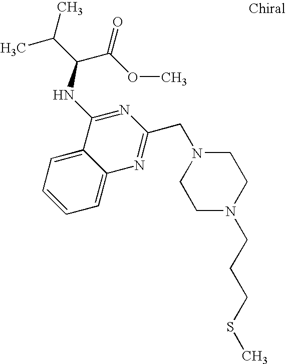 | 17-13 | 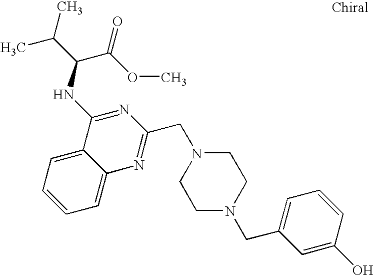 |
| 17-7 | 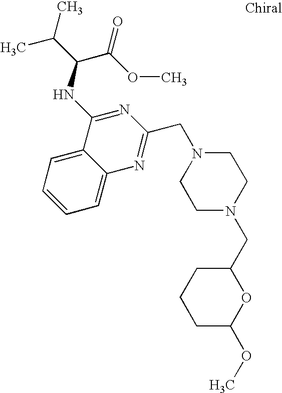 | 17-14 | 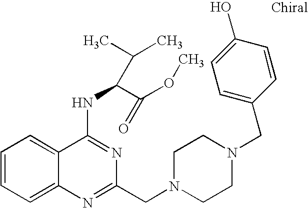 |
| 17-15 | 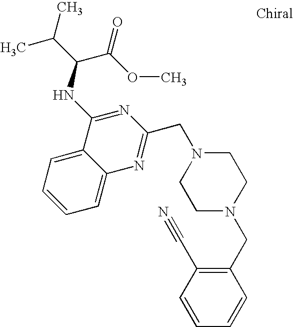 | 17-22 | 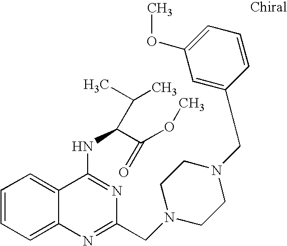 |
| 17-16 | 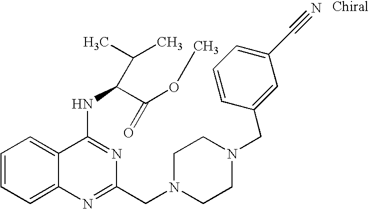 | 17-23 | 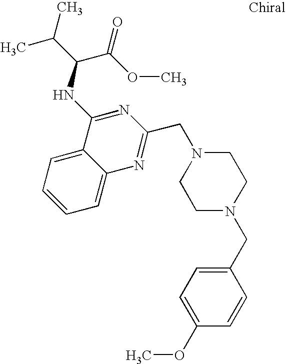 |
| 17-17 | 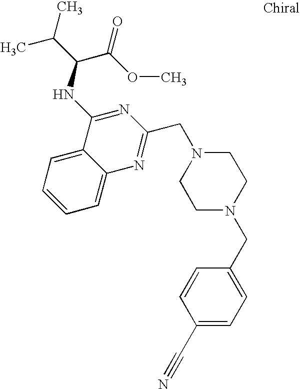 | 17-24 | 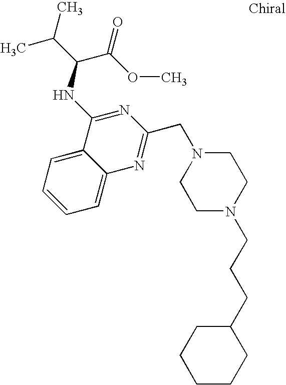 |
| 17-18 | 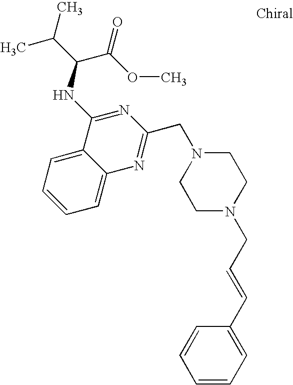 | 17-25 | 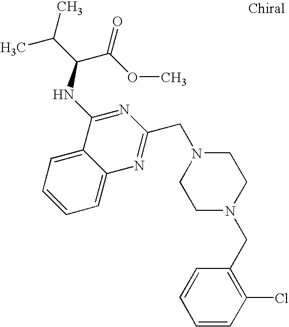 |
| 17-19 | 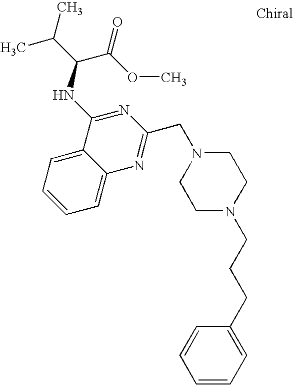 | 17-26 | 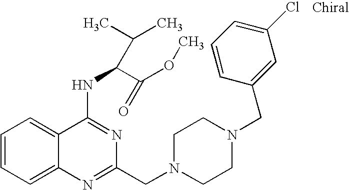 |
| 17-20 | 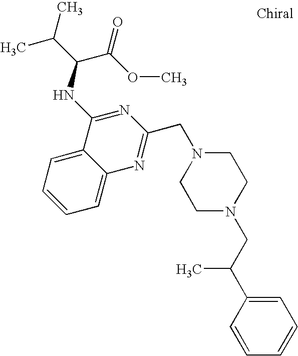 | 17-27 | 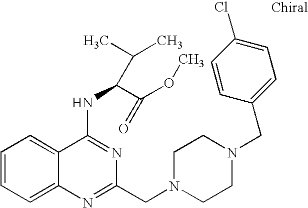 |
| 17-21 | 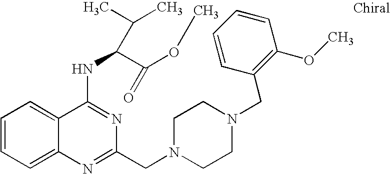 | 17-28 | 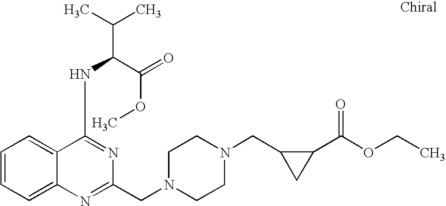 |
| 17-29 | 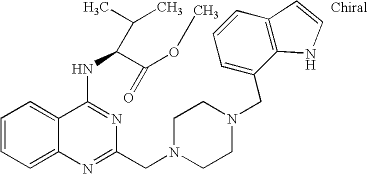 | 17-36 | 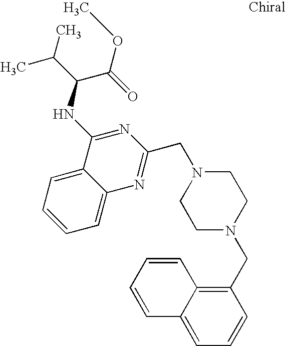 |
| 17-30 | 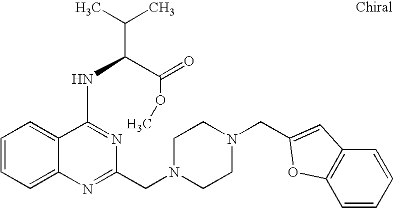 | 17-37 | 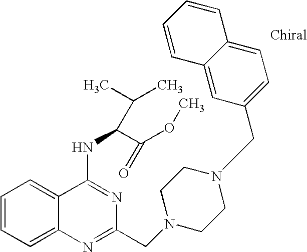 |
| 17-31 | 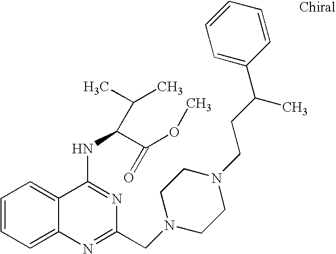 | 17-38 | 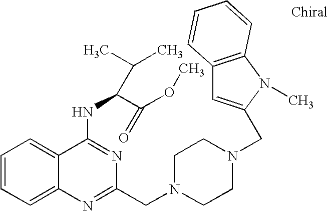 |
| 17-32 | 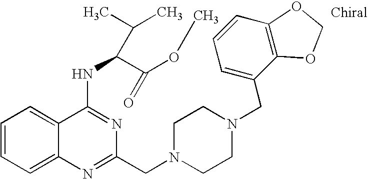 | 17-39 | 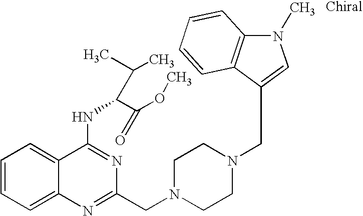 |
| 17-33 | 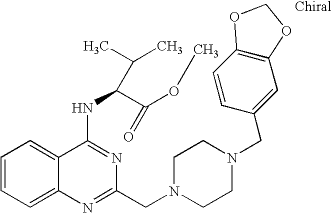 | 17-40 | 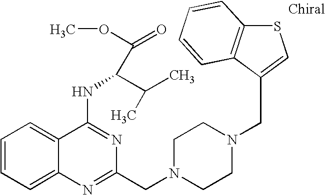 |
| 17-34 | 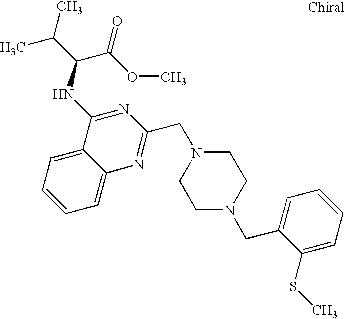 | 17-41 | 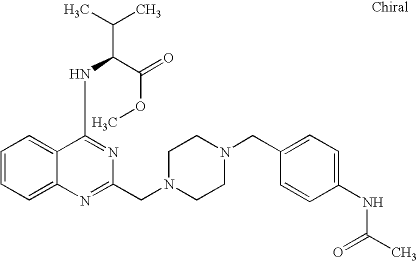 |
| 17-35 | 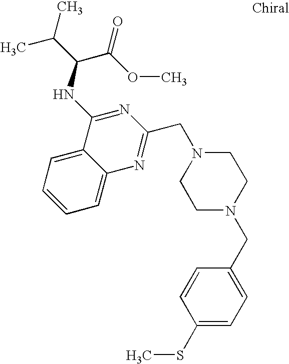 | 17-42 | 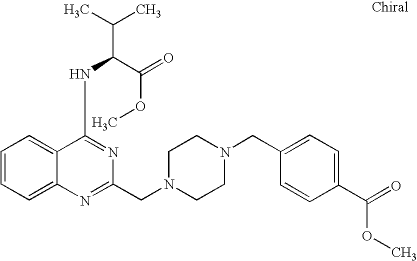 |
| 17-43 | 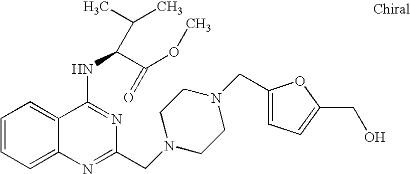 | 17-50 | 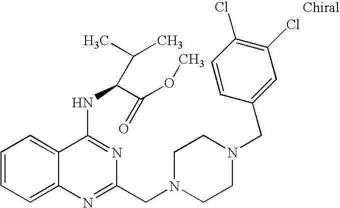 |
| 17-44 | 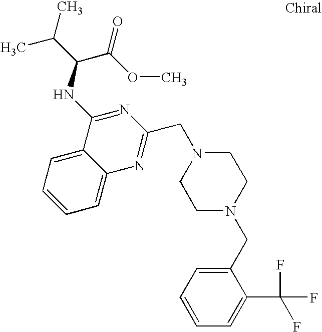 | 17-51 | 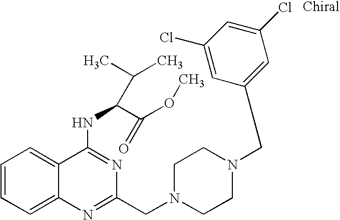 |
| 17-45 | 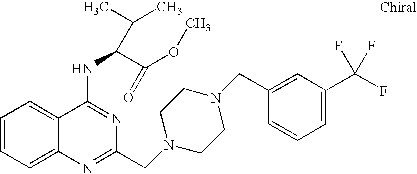 | 17-52 | 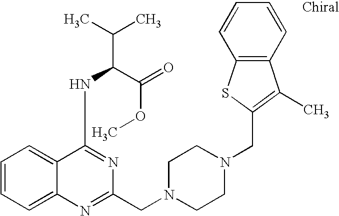 |
| 17-46 | 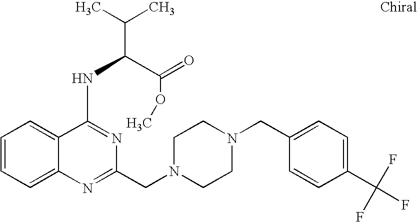 | 17-53 | 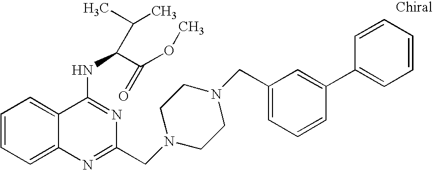 |
| 17-47 | 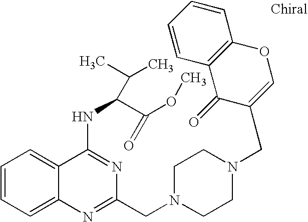 | 17-54 | 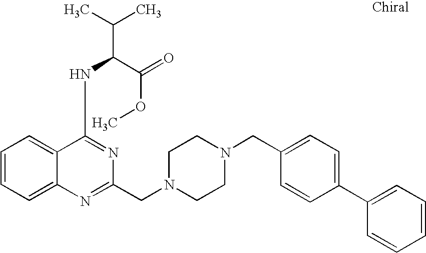 |
| 17-48 | 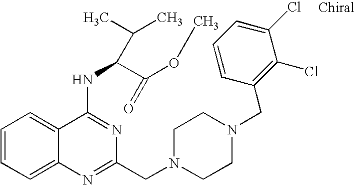 | 17-55 | 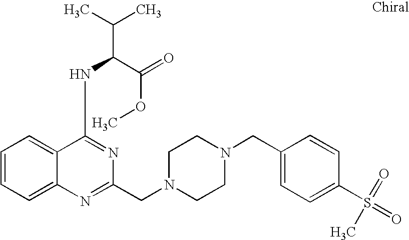 |
| 17-49 | 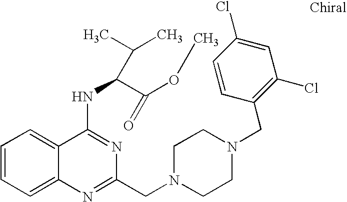 | 17-56 | 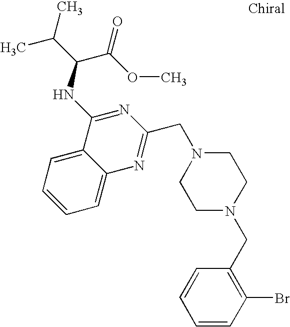 |
| 17-57 | 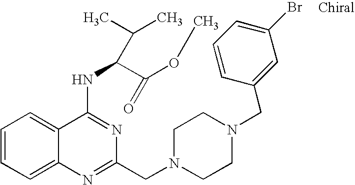 | 17-64 | 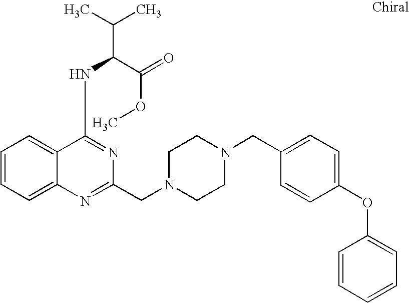 |
| 17-58 | 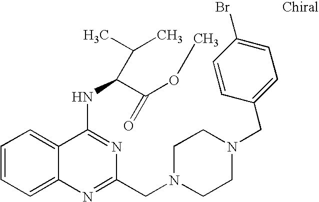 | 17-65 | 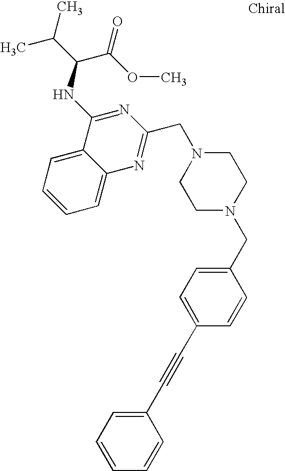 |
| 17-59 | 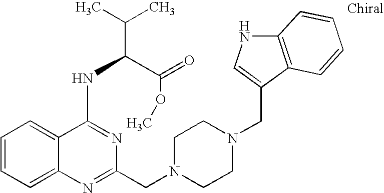 | 17-66 | 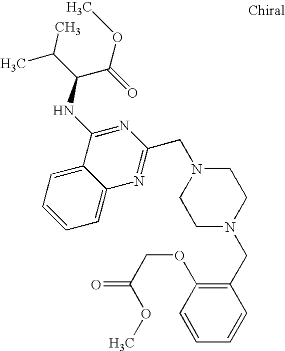 |
| 17-60 | 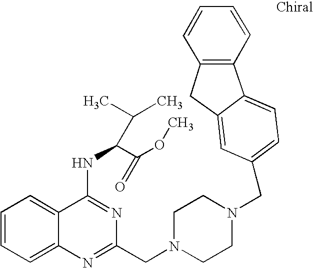 | 17-67 | 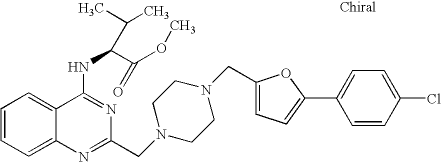 |
| 17-61 | 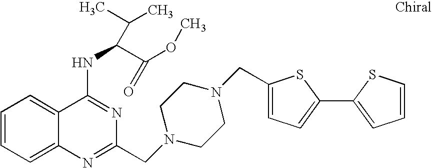 | 17-68 | 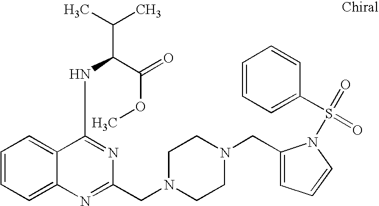 |
| 17-62 | 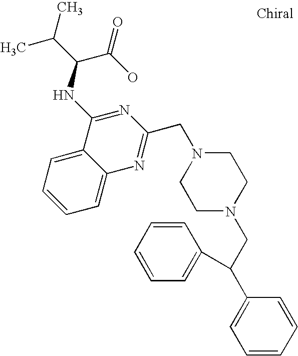 | 17-69 | 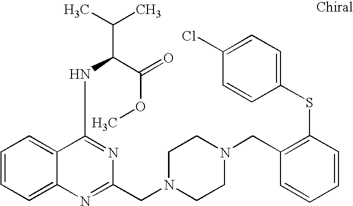 |
| 17-63 | 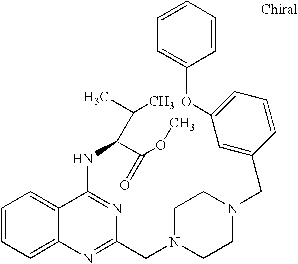 | 17-70 | 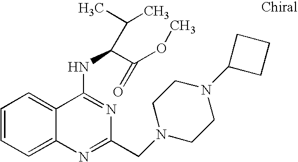 |
| 17-71 | 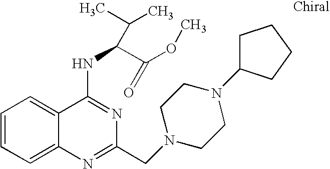 | 17-78 | 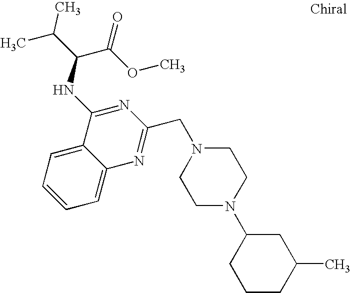 |
| 17-72 | 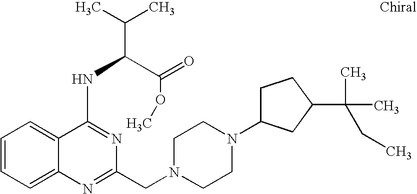 | 17-79 | 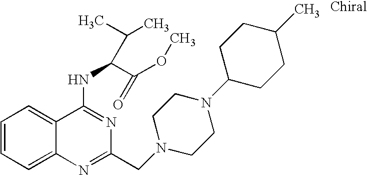 |
| 17-73 | 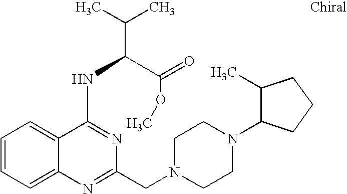 | 17-80 | 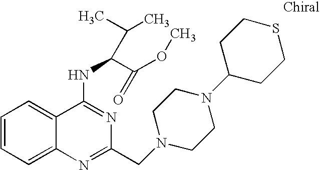 |
| 17-74 | 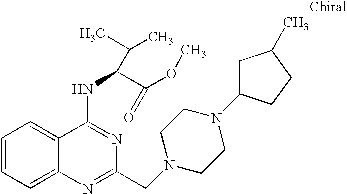 | 17-81 | 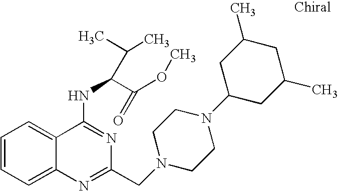 |
| 17-75 | 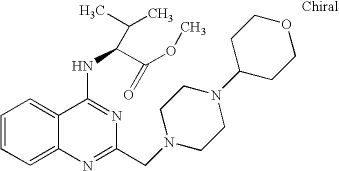 | 17-82 | 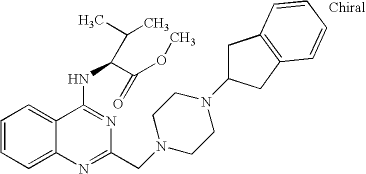 |
| 17-76 | 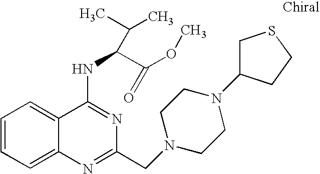 | 17-83 | 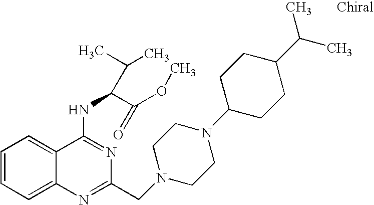 |
| 17-77 | 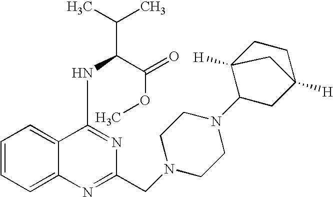 | 17-84 | 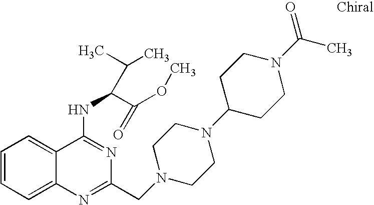 |
| 17-85 | 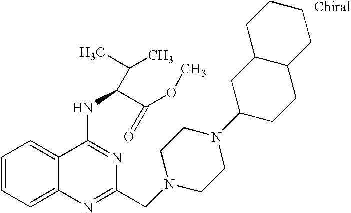 | 17-92 | 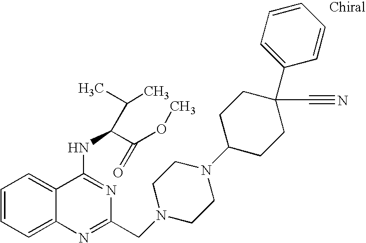 |
| 17-86 | 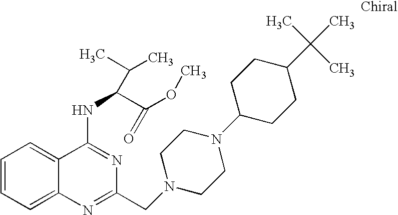 | 17-93 | 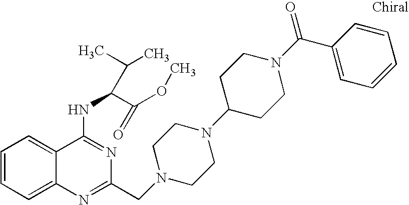 |
| 17-87 | 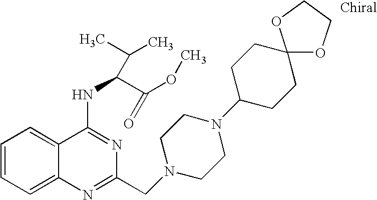 | 17-94 | 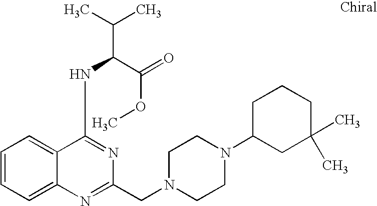 |
| 17-88 | 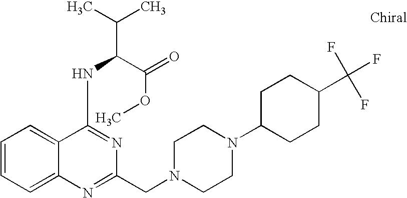 | 17-95 | 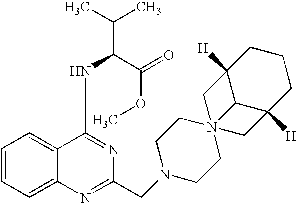 |
| 17-89 | 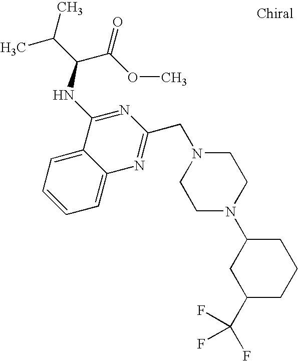 | 18-1 | 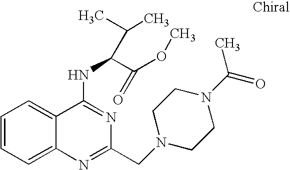 |
| 17-90 | 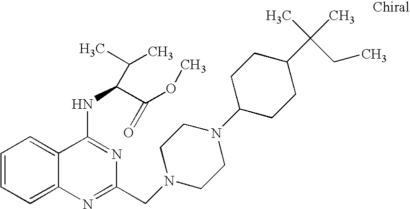 | 18-2 | 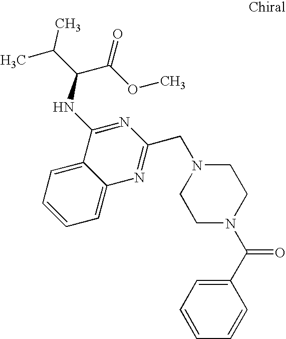 |
| 17-91 | 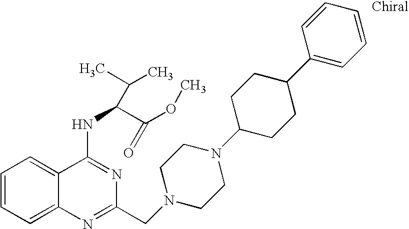 | 18-3 | 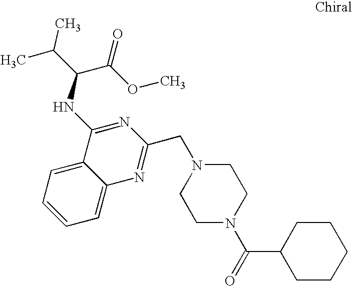 |
| 18-4 | 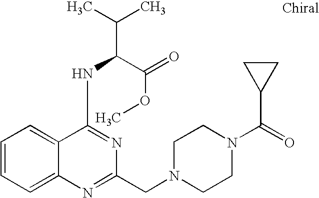 | 18-11 | 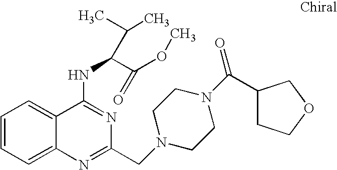 |
| 18-5 | 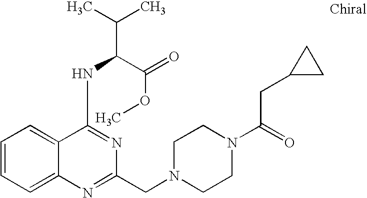 | 18-12 | 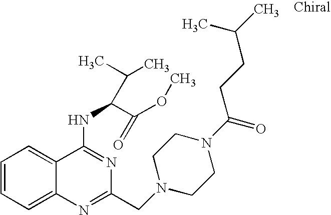 |
| 18-6 | 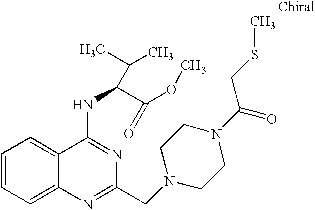 | 18-13 | 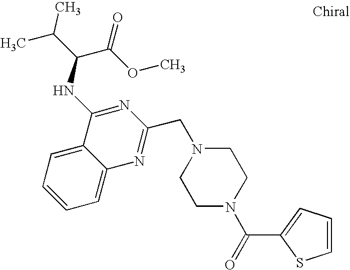 |
| 18-7 | 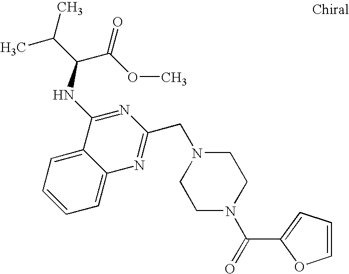 | 18-14 | 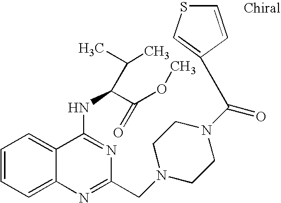 |
| 18-8 | 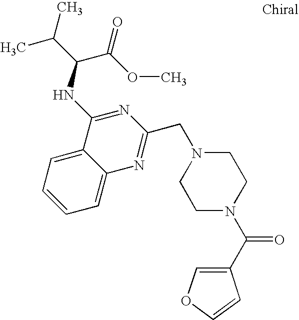 | 18-15 | 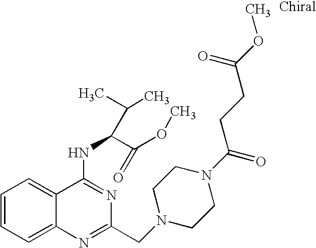 |
| 18-9 | 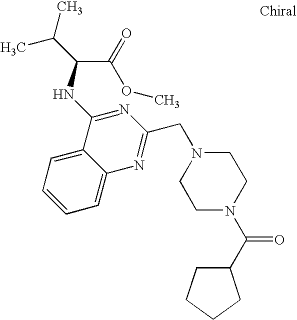 | 18-16 | 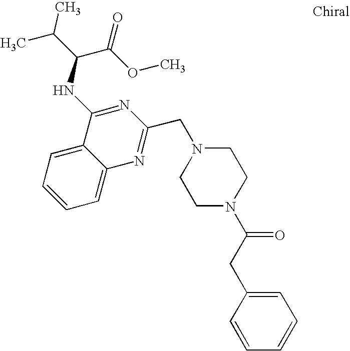 |
| 18-10 | 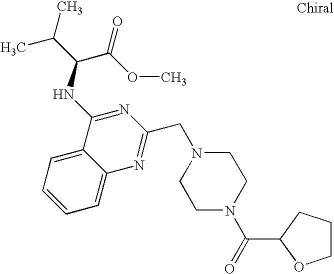 | 18-17 | 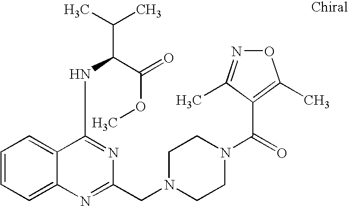 |
| 18-18 | 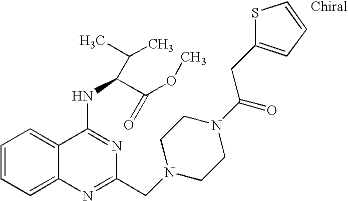 | 18-25 | 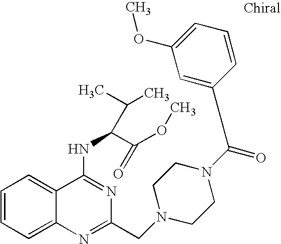 |
| 18-19 | 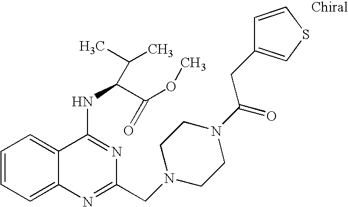 | 18-26 | 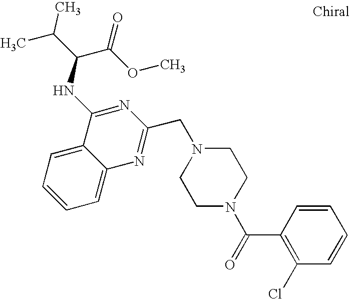 |
| 18-20 | 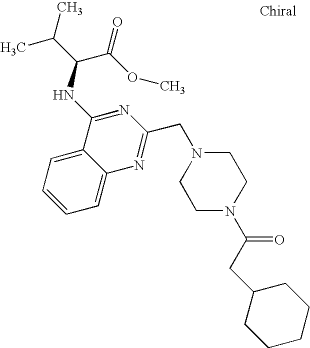 | 18-27 | 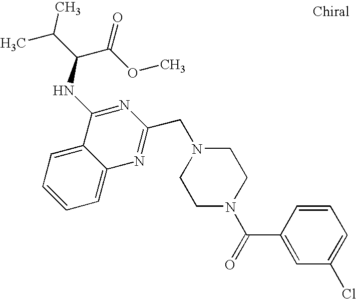 |
| 18-21 | 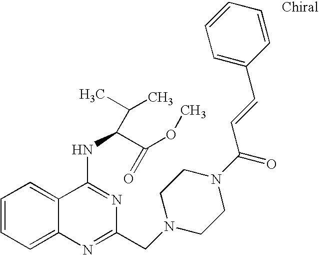 | 18-28 | 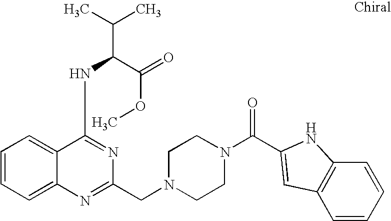 |
| 18-22 | 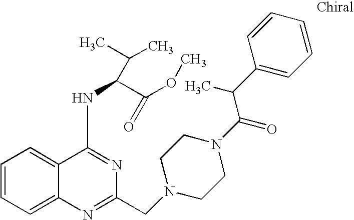 | 18-29 | 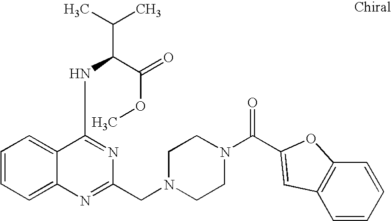 |
| 18-23 | 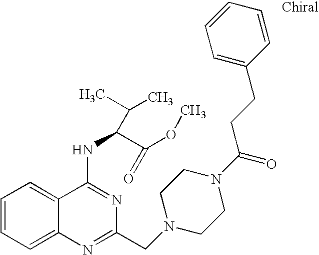 | 18-30 | 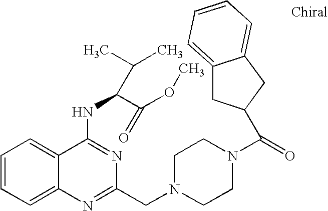 |
| 18-24 | 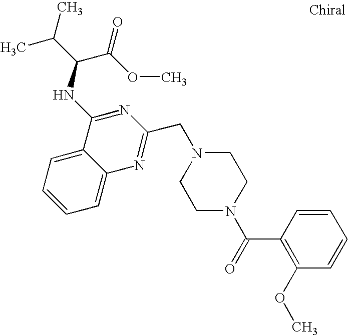 | 18-31 | 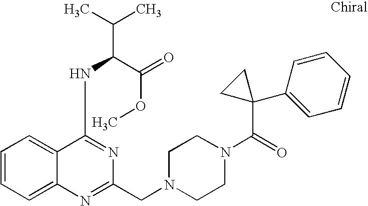 |
| 18-32 | 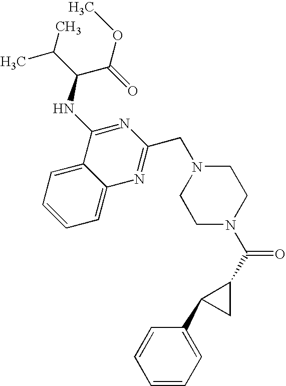 | 18-39 | 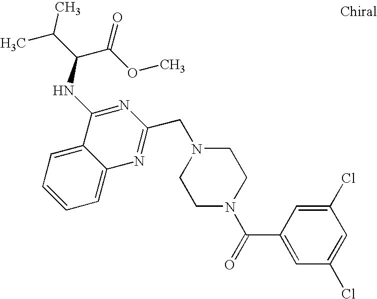 |
| 18-33 | 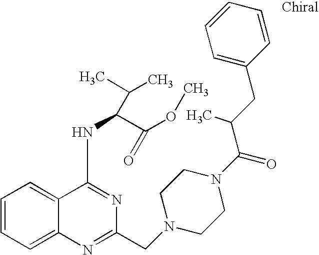 | 18-40 | 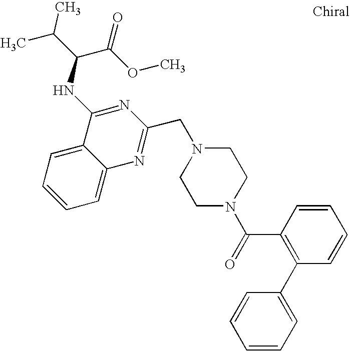 |
| 18-34 | 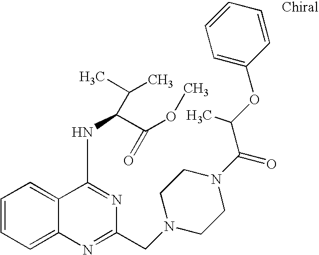 | 18-41 | 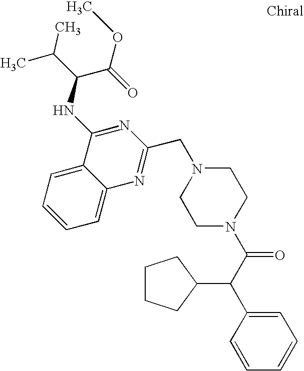 |
| 18-35 | 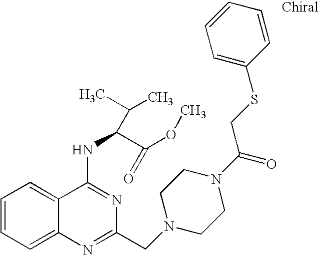 | 18-42 | 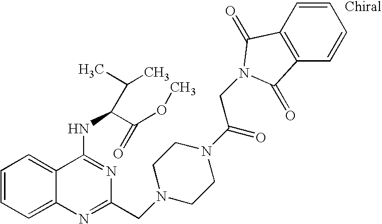 |
| 18-36 | 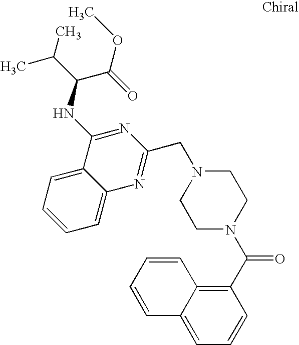 | 18-43 | 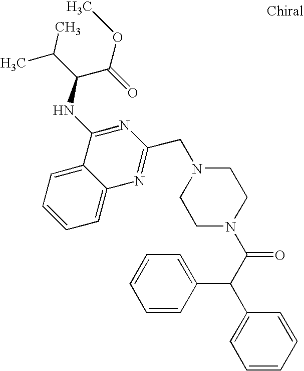 |
| 18-37 | 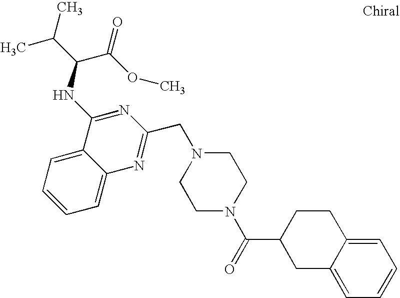 | 18-44 | 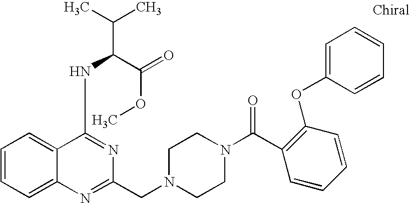 |
| 18-38 | 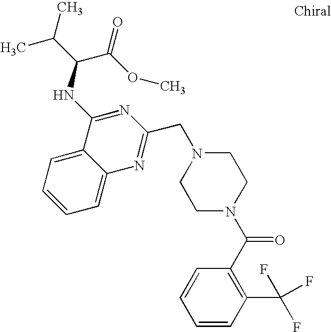 | 18-45 | 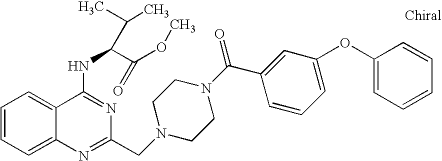 |
| 18-46 | 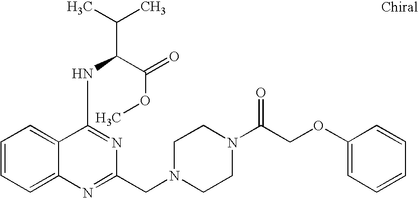 | 18-53 | 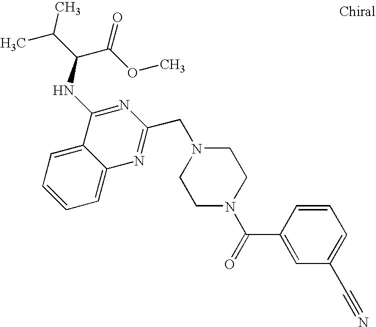 |
| 18-47 | 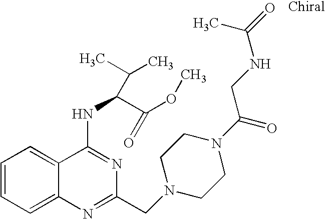 | 18-54 | 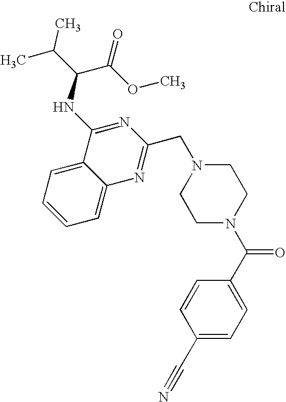 |
| 18-48 | 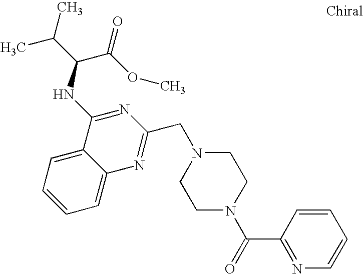 | 18-55 | 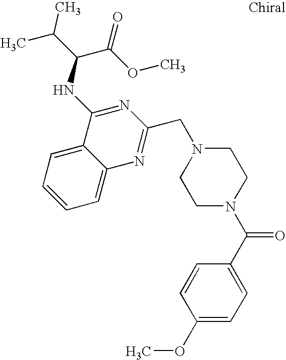 |
| 18-49 | 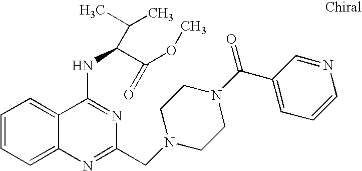 | 18-56 | 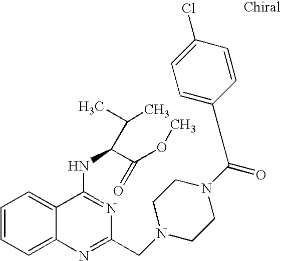 |
| 18-50 | 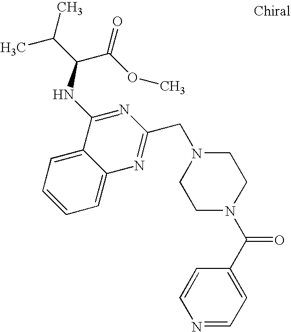 | 18-57 | 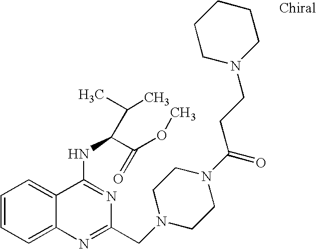 |
| 18-51 | 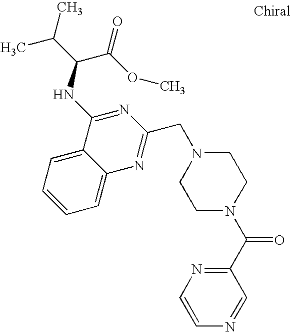 | 18-58 | 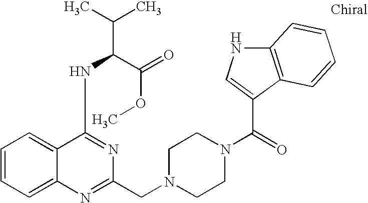 |
| 18-52 | 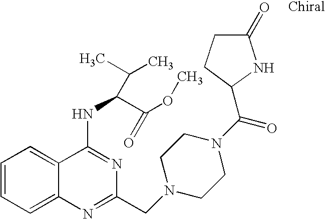 | 18-59 | 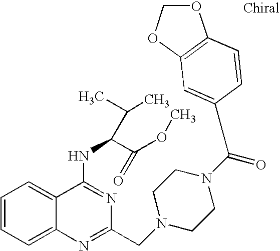 |
| 18-60 | 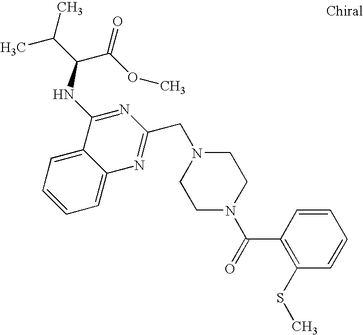 | 18-67 | 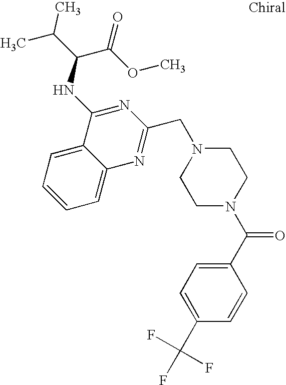 |
| 18-61 | 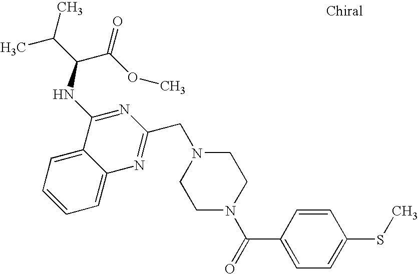 | 18-68 | 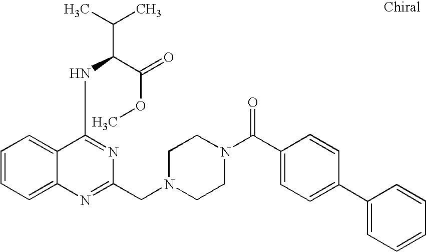 |
| 18-62 | 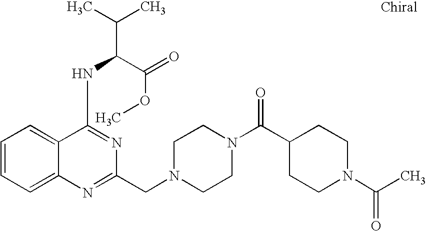 | 18-69 | 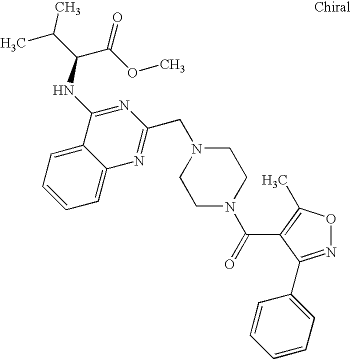 |
| 18-63 | 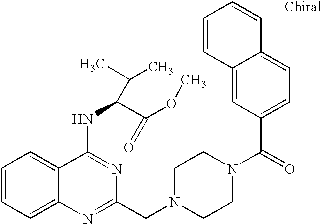 | 18-70 | 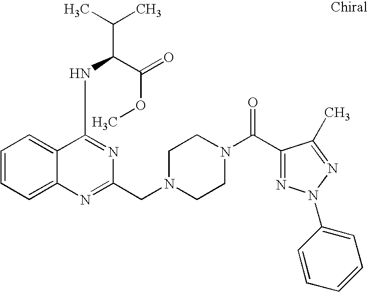 |
| 18-64 | 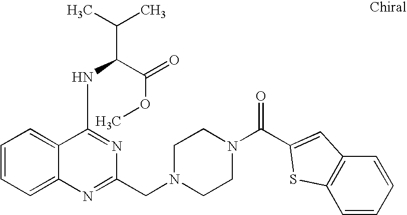 | 18-71 | 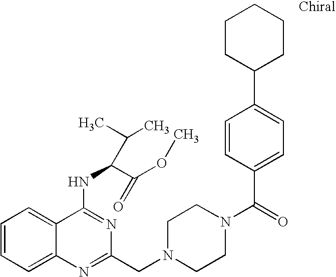 |
| 18-65 | 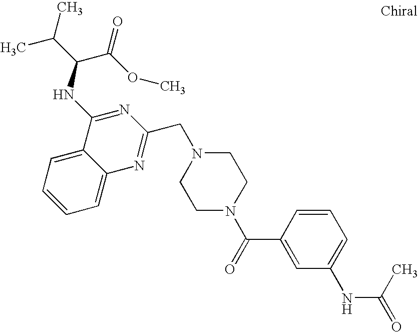 | 18-72 | 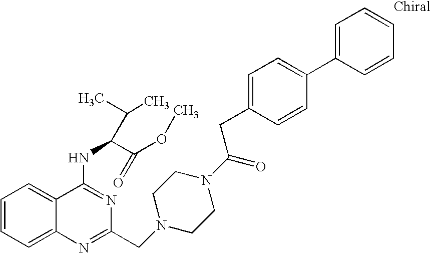 |
| 18-66 | 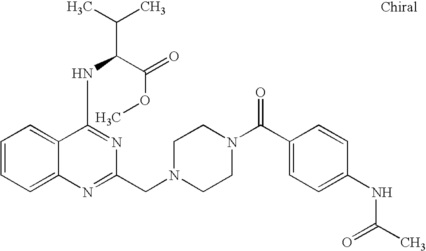 | 19-1 | 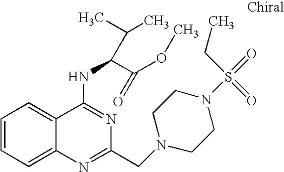 |
| 19-2 | 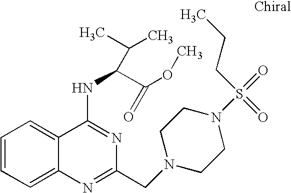 | 19-9 | 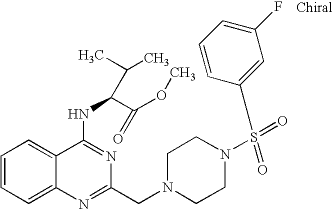 |
| 19-3 | 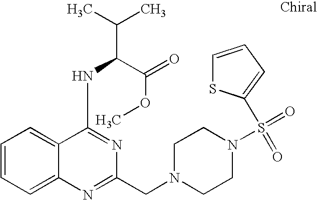 | 19-10 | 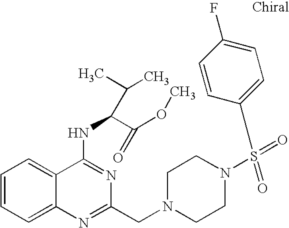 |
| 19-4 | 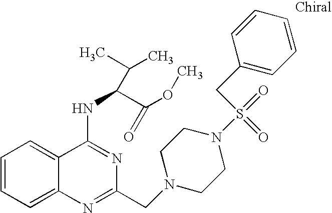 | 19-11 | 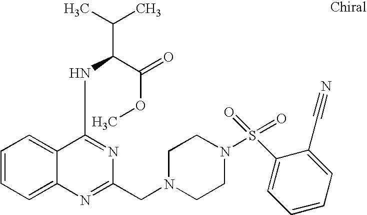 |
| 19-5 | 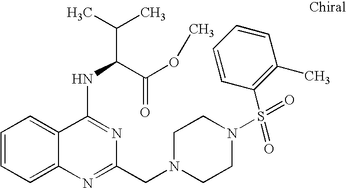 | 19-12 | 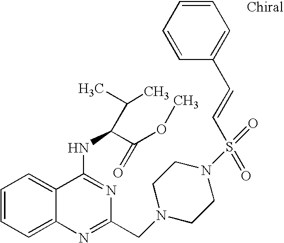 |
| 19-6 | 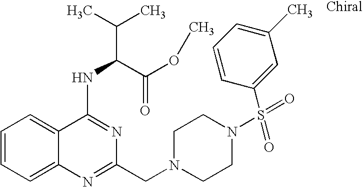 | 19-13 | 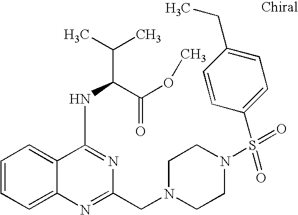 |
| 19-7 | 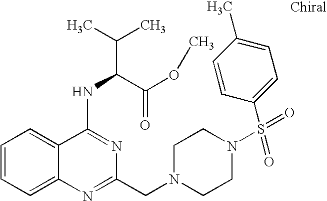 | 19-14 | 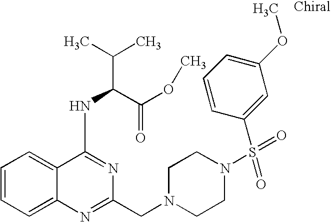 |
| 19-8 | 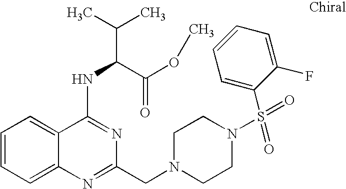 | 19-15 | 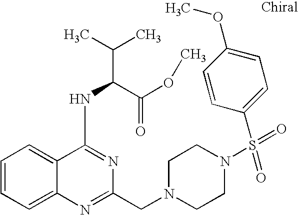 |
| 19-16 | 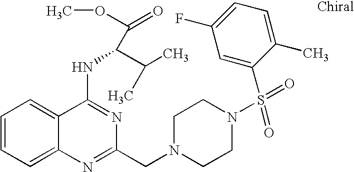 | 19-23 | 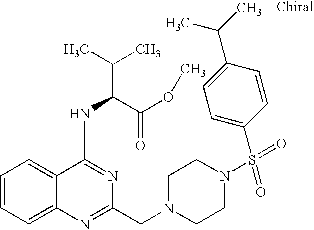 |
| 19-17 | 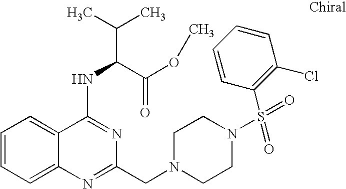 | 19-24 | 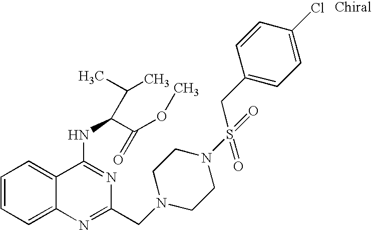 |
| 19-18 | 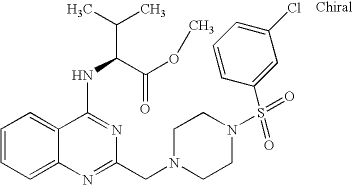 | 19-25 | 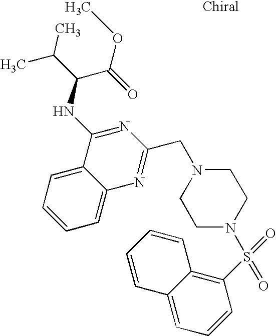 |
| 19-19 | 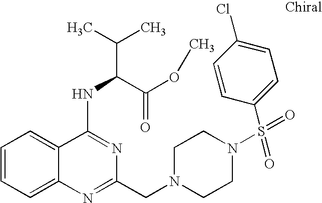 | 19-26 | 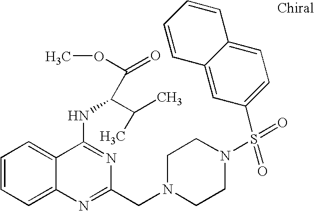 |
| 19-20 | 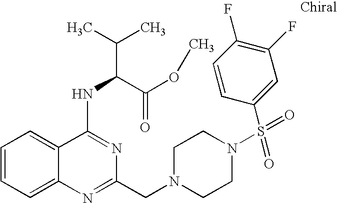 | 19-27 | 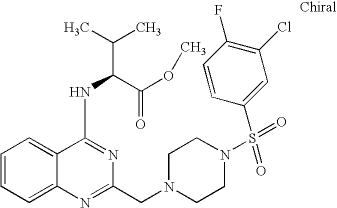 |
| 19-21 | 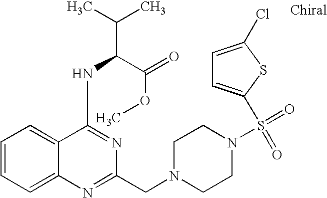 | 19-28 | 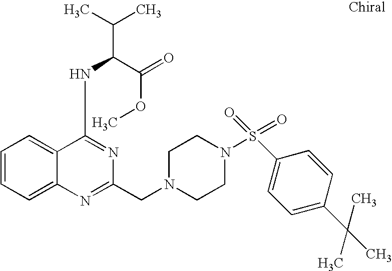 |
| 19-22 | 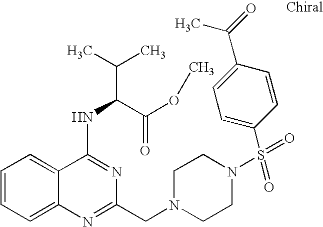 | 19-29 | 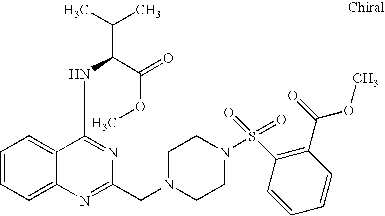 |
| 19-30 | 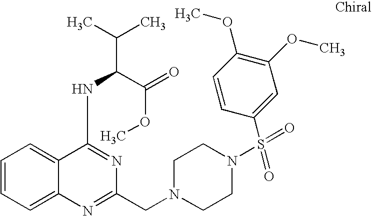 | 19-37 | 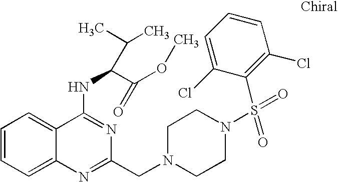 |
| 19-31 | 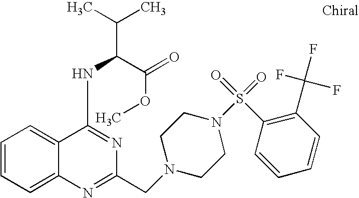 | 19-38 | 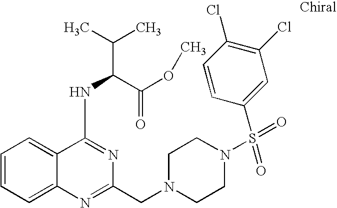 |
| 19-32 | 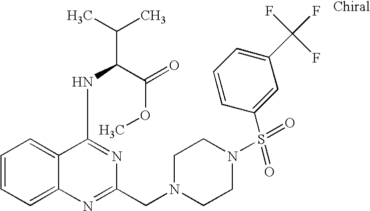 | 19-39 | 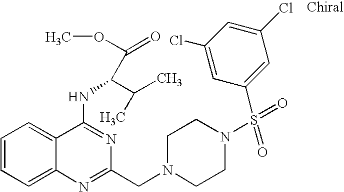 |
| 19-33 | 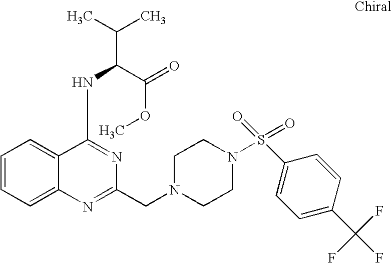 | 19-40 | 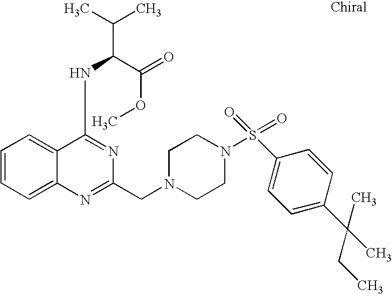 |
| 19-34 | 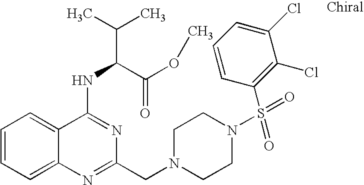 | 19-41 | 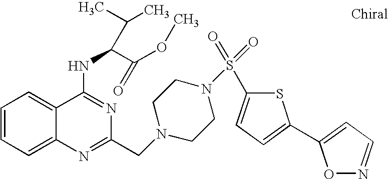 |
| 19-35 | 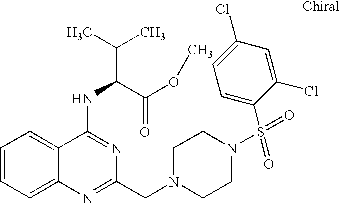 | 19-42 | 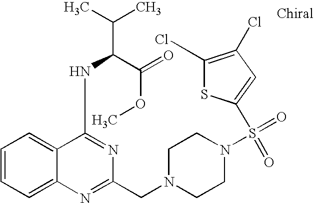 |
| 19-36 | 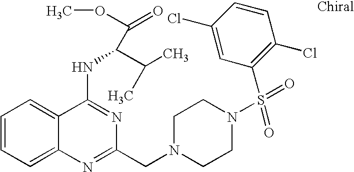 | 19-43 | 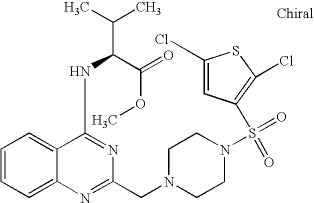 |
| 19-44 | 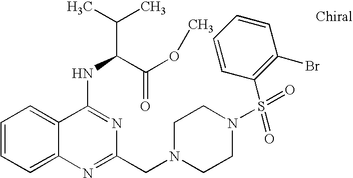 | 19-51 | 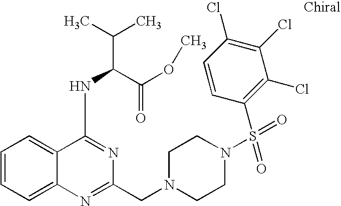 |
| 19-45 | 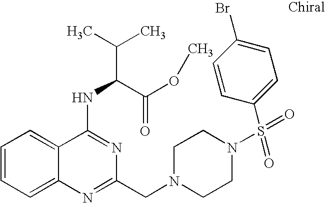 | 19-52 | 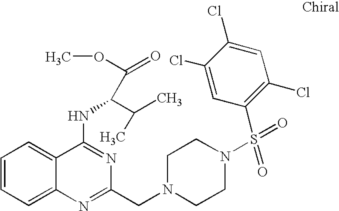 |
| 19-46 | 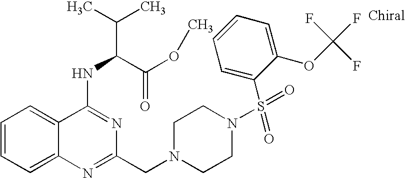 | 19-53 | 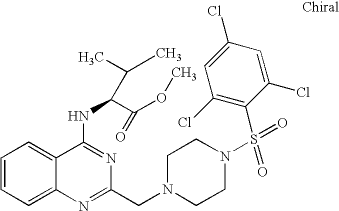 |
| 19-47 | 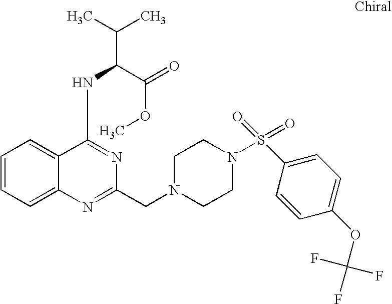 | 19-54 | 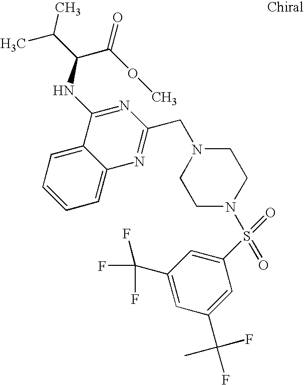 |
| 19-48 | 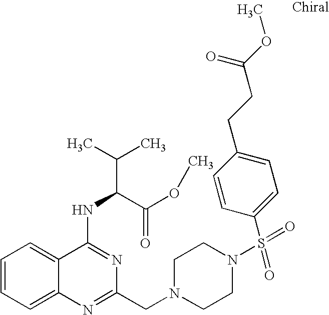 | 19-55 | 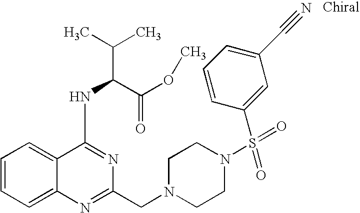 |
| 19-49 | 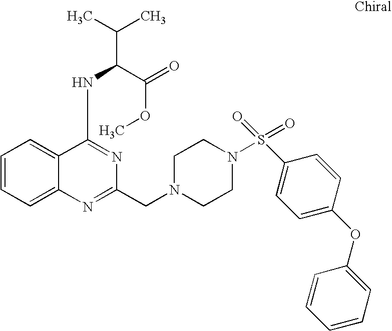 | 19-56 | 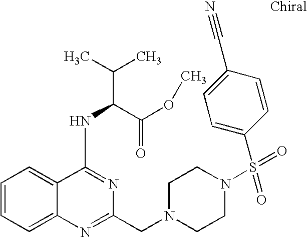 |
| 19-50 | 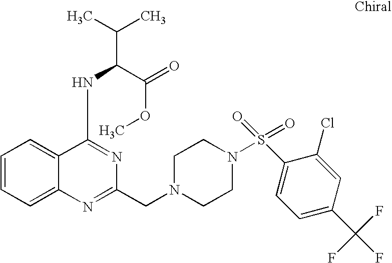 | 19-57 | 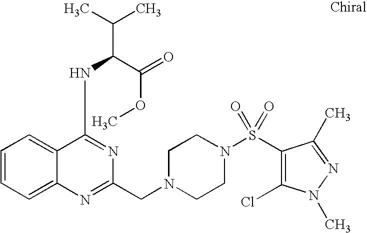 |
| 19-58 | 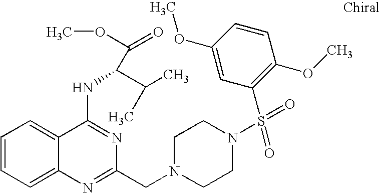 | 19-65 | 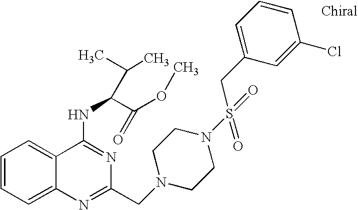 |
| 19-59 | 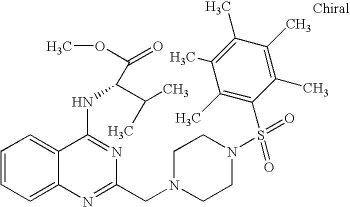 | 19-66 | 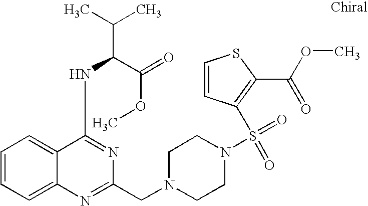 |
| 19-60 | 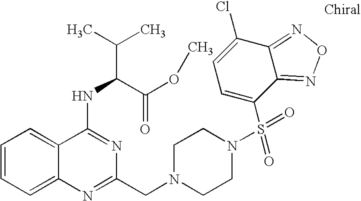 | 19-67 | 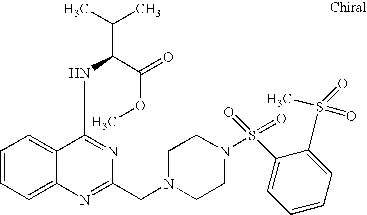 |
| 19-61 | 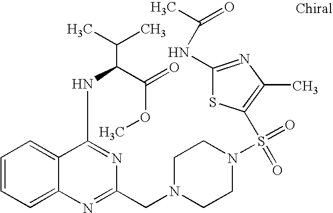 | 19-68 | 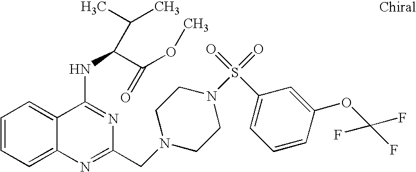 |
| 19-62 | 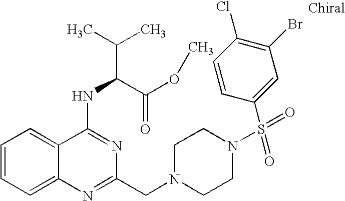 | 19-69 | 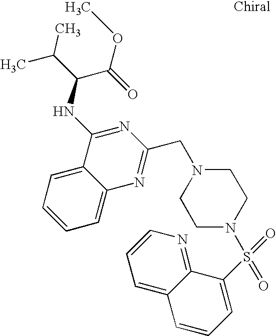 |
| 19-63 | 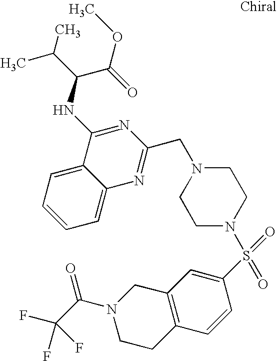 | 19-70 | 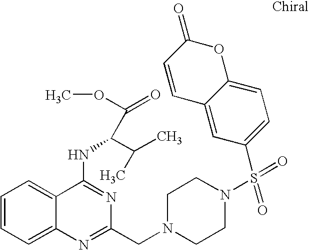 |
| 19-64 | 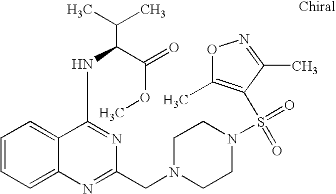 | 19-71 | 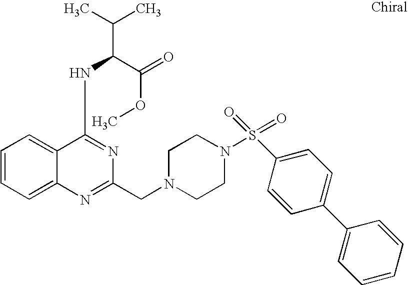 |
| 19-72 | 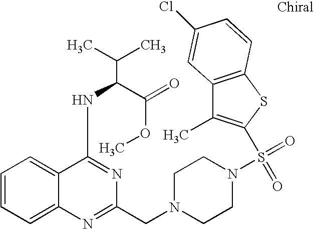 | 20-3 | 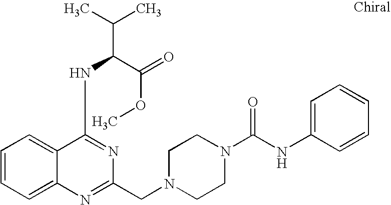 |
| 19-73 | 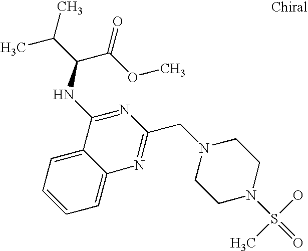 | 20-4 | 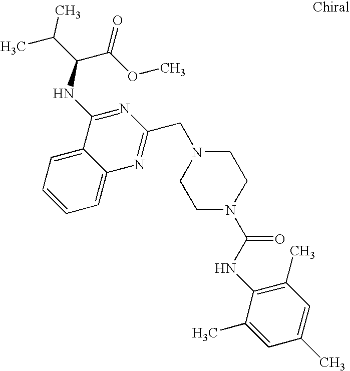 |
| 19-74 | 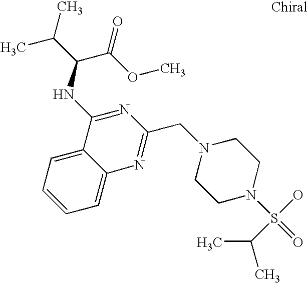 | 20-5 | 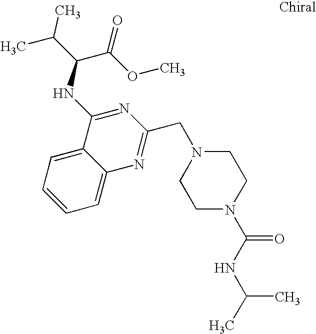 |
| 19-75 | 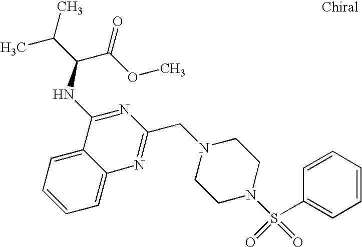 | 20-6 | 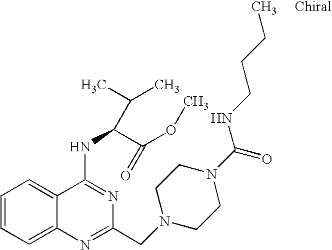 |
| 19-76 | 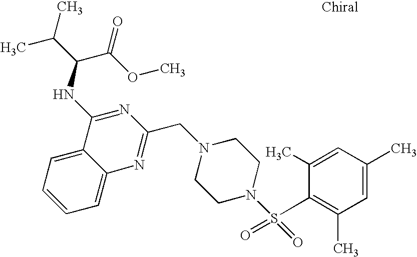 | 20-7 | 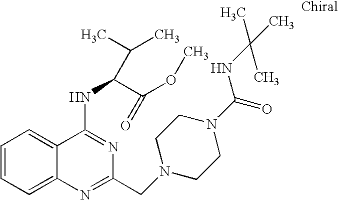 |
| 20-1 | 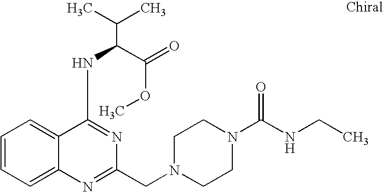 | 20-8 | 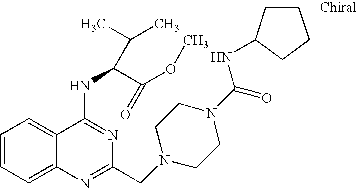 |
| 20-2 | 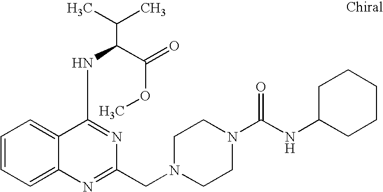 | 20-9 | 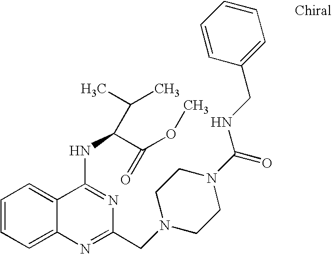 |
| 20-10 | 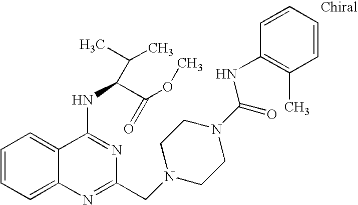 | 20-17 | 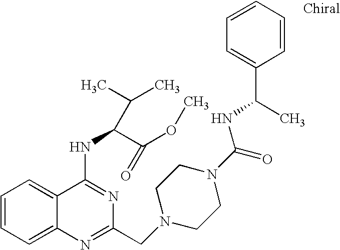 |
| 20-11 | 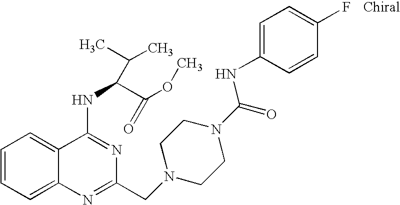 | 20-18 | 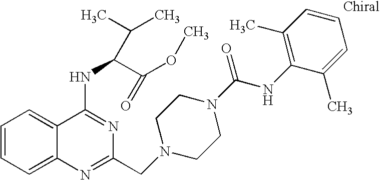 |
| 20-12 | 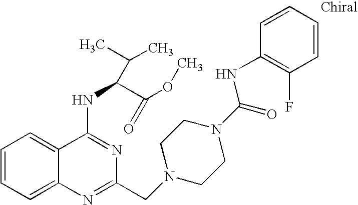 | 20-19 | 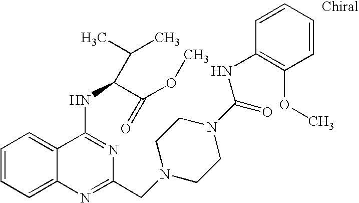 |
| 20-13 | 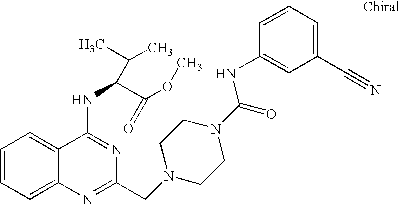 | 20-20 | 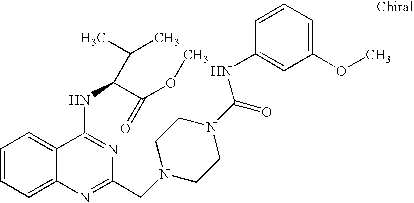 |
| 20-14 | 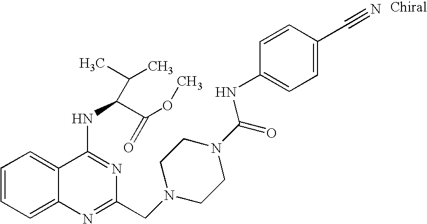 | 20-21 | 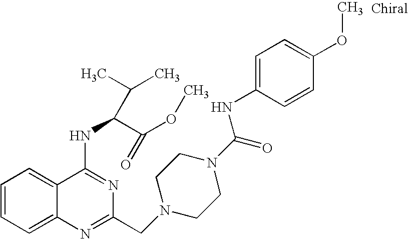 |
| 20-15 | 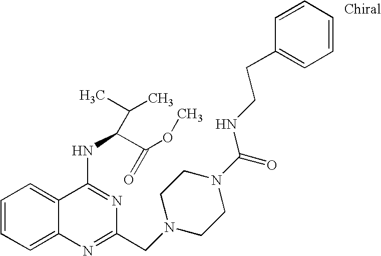 | 20-22 | 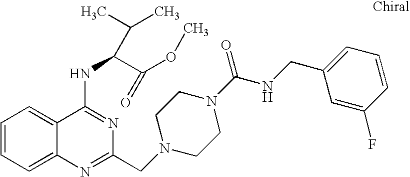 |
| 20-16 | 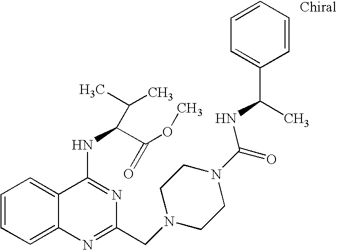 | 20-23 | 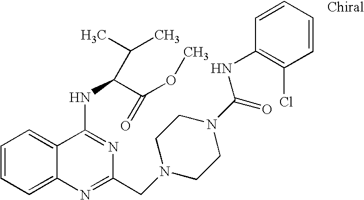 |
| 20-24 | 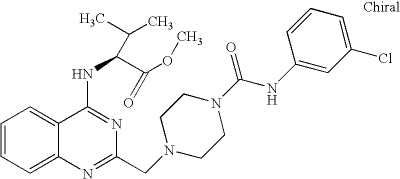 | 20-31 | 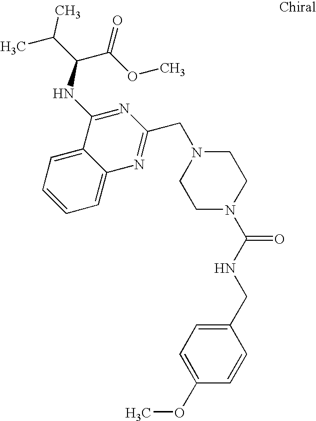 |
| 20-25 | 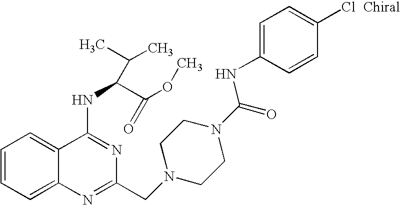 | 20-32 | 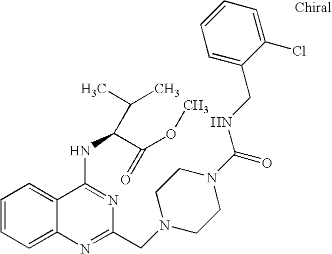 |
| 20-26 | 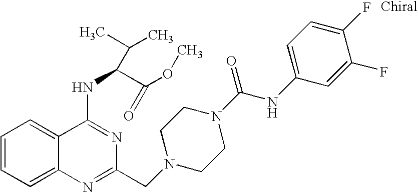 | 20-33 | 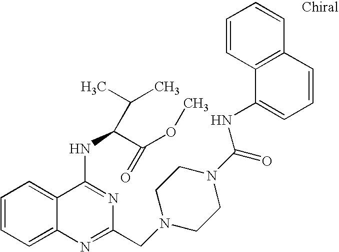 |
| 20-27 | 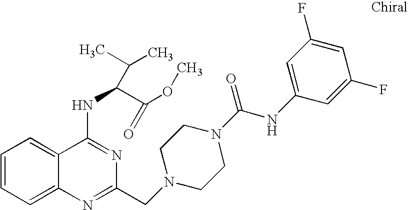 | 20-34 | 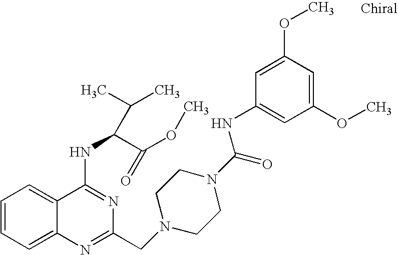 |
| 20-28 | 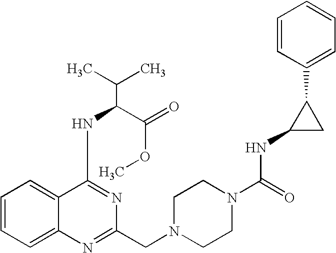 | 20-35 | 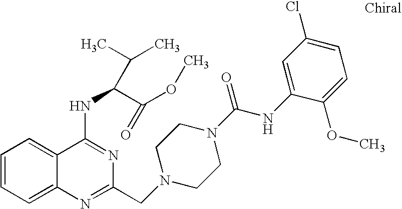 |
| 20-29 | 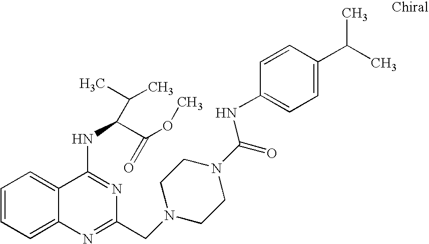 | 20-36 | 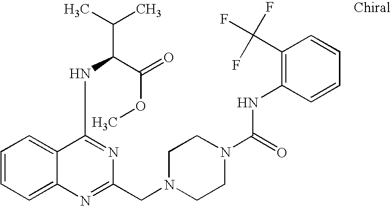 |
| 20-30 | 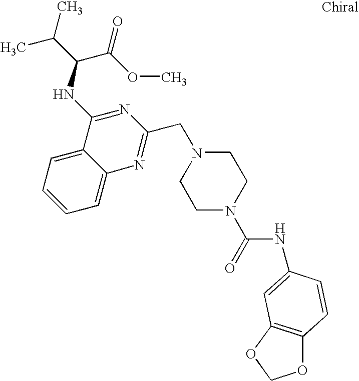 | 20-37 | 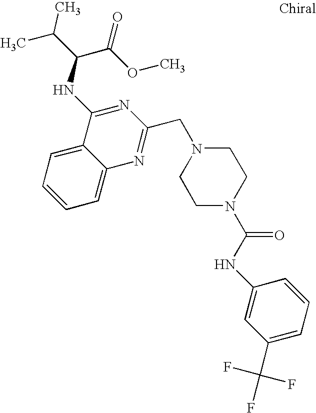 |
| 20-38 | 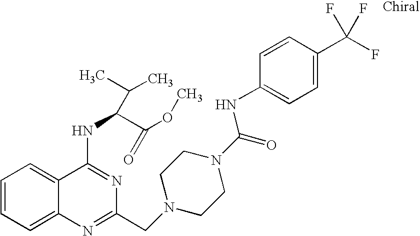 | 20-45 | 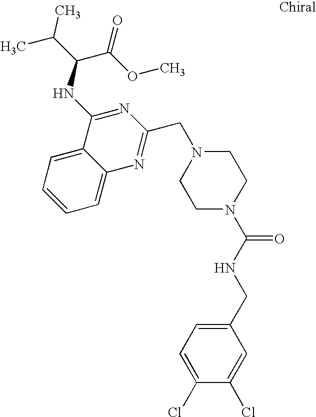 |
| 20-39 | 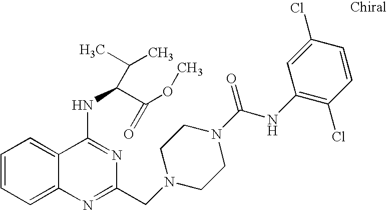 | 20-46 | 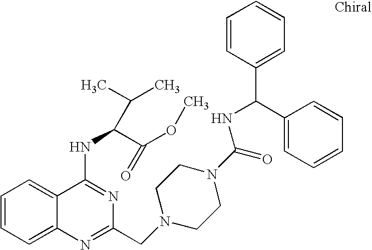 |
| 20-40 | 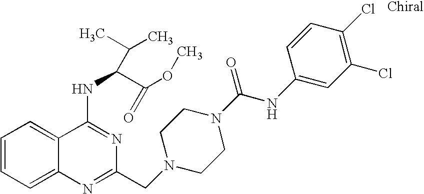 | 20-47 | 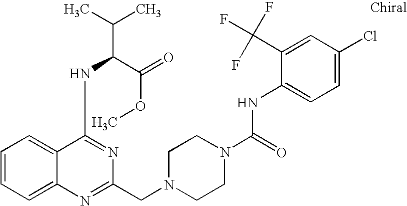 |
| 20-41 | 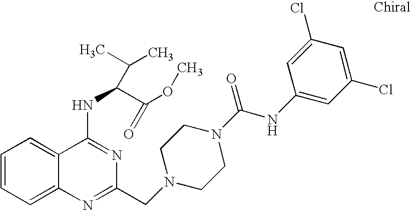 | 20-48 | 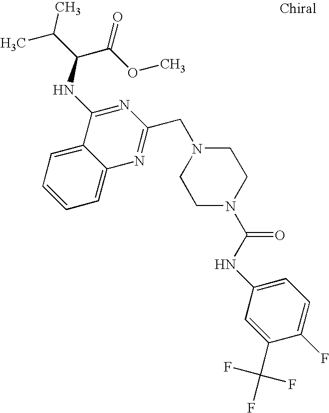 |
| 20-42 | 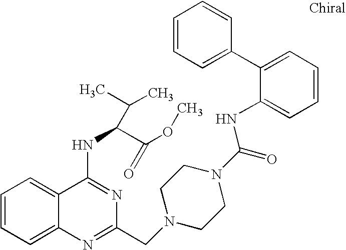 | 20-49 | 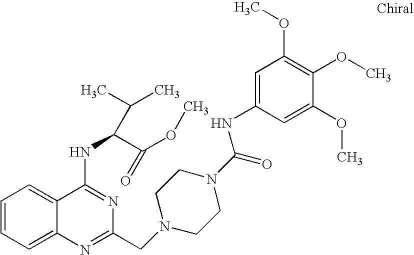 |
| 20-43 | 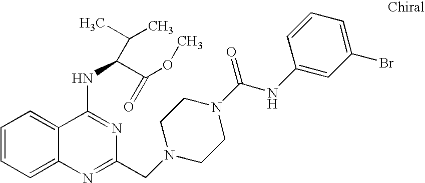 | 20-50 | 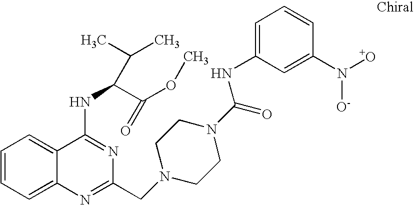 |
| 20-44 | 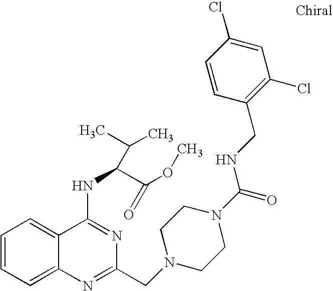 | 21-1 | 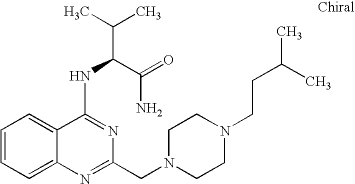 |
| 21-2 | 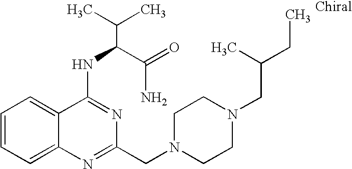 | 21-9 | 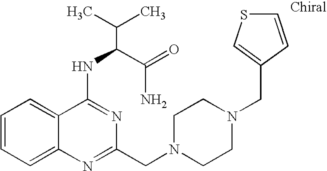 |
| 21-3 | 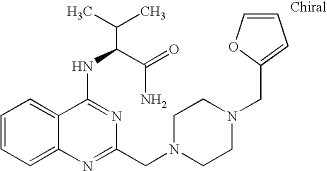 | 21-10 | 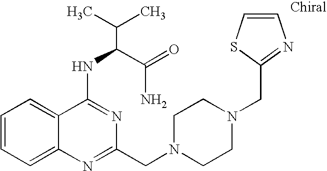 |
| 21-4 | 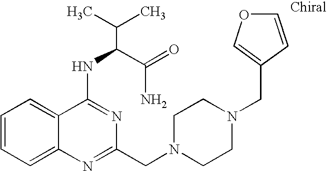 | 21-11 | 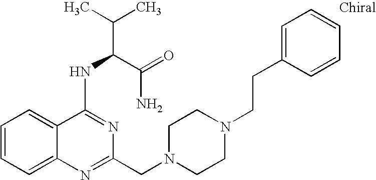 |
| 21-5 | 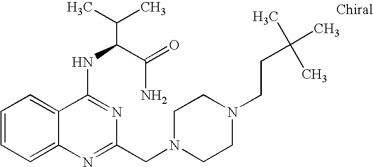 | 21-12 | 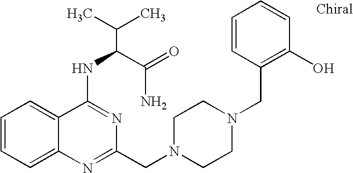 |
| 21-6 | 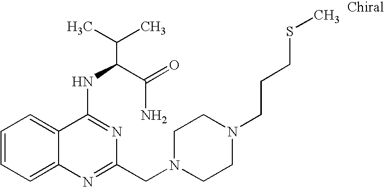 | 21-13 | 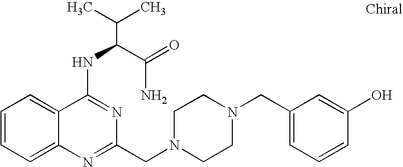 |
| 21-7 | 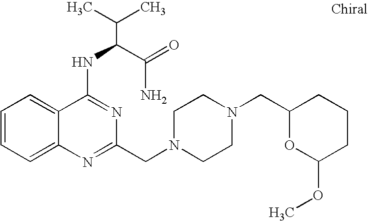 | 21-14 | 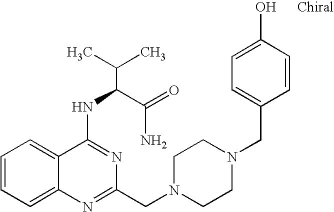 |
| 21-8 | 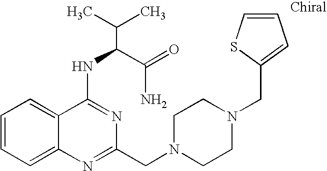 | 21-15 | 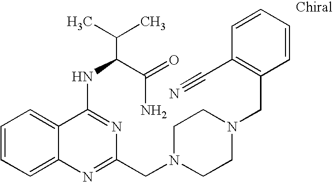 |
| 21-16 | 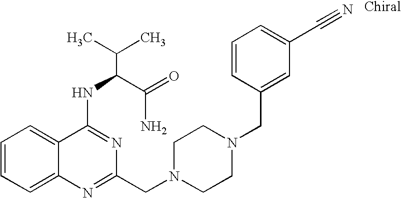 | 21-23 | 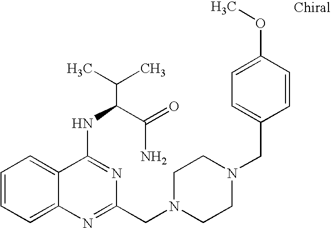 |
| 21-17 | 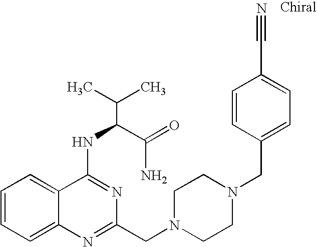 | 21-24 | 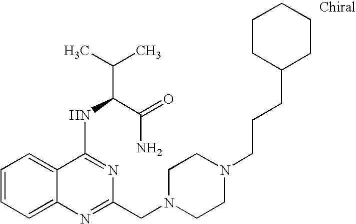 |
| 21-18 | 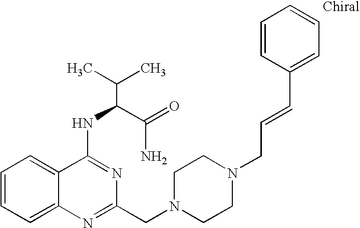 | 21-25 | 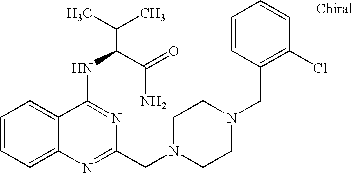 |
| 21-19 | 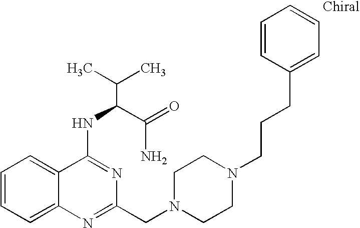 | 21-26 | 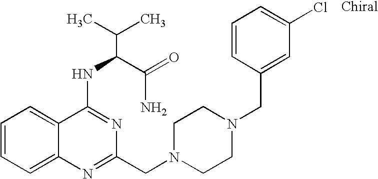 |
| 21-20 | 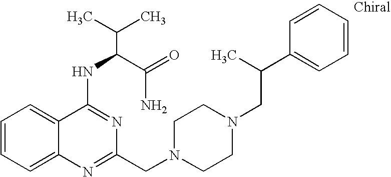 | 21-27 | 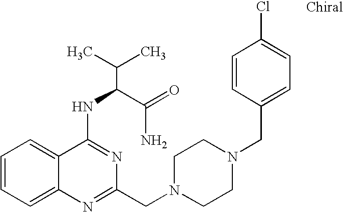 |
| 21-21 | 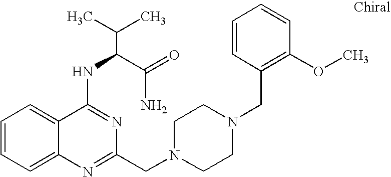 | 21-28 | 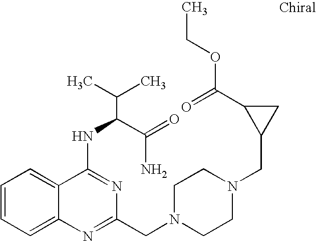 |
| 21-22 | 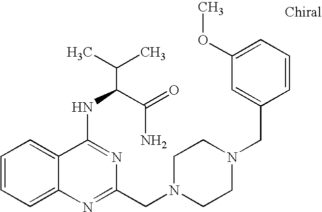 | 21-29 | 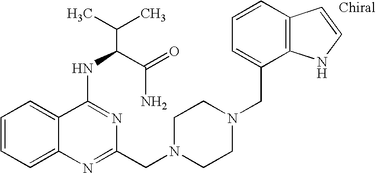 |
| 21-30 | 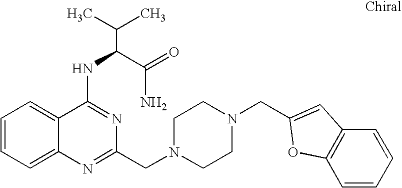 | 21-37 | 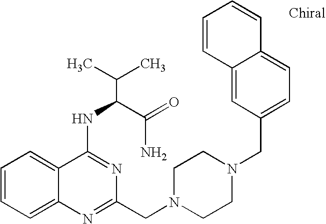 |
| 21-31 | 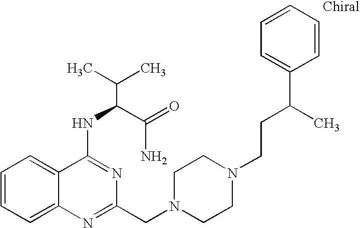 | 21-38 | 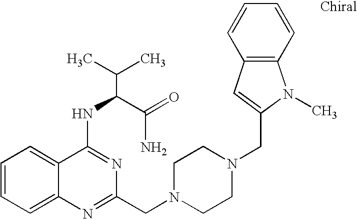 |
| 21-32 | 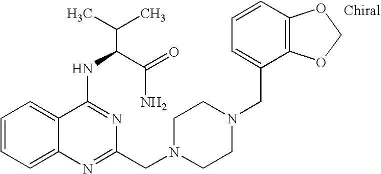 | 21-39 | 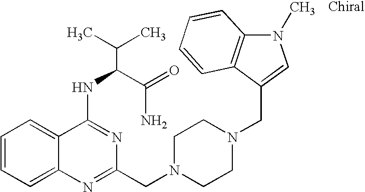 |
| 21-33 | 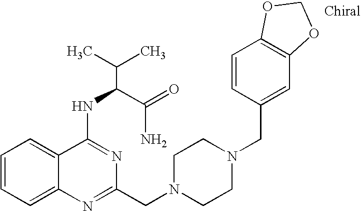 | 21-40 | 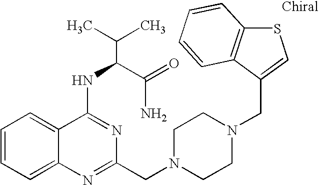 |
| 21-34 | 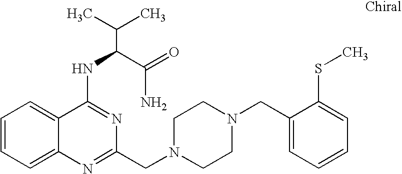 | 21-41 | 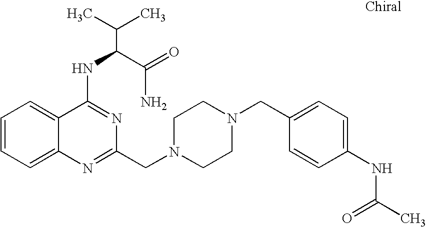 |
| 21-35 | 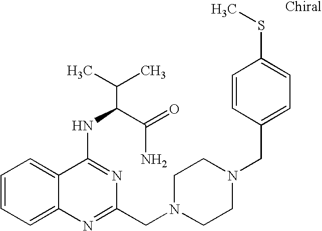 | 21-42 | 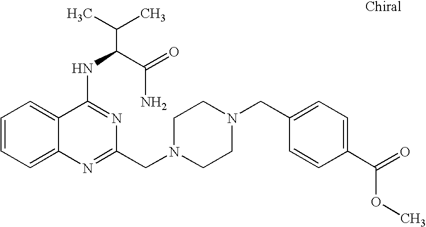 |
| 21-36 | 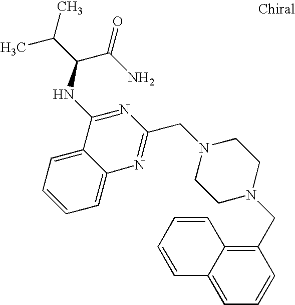 | 21-43 | 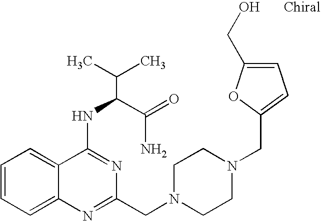 |
| 21-44 | 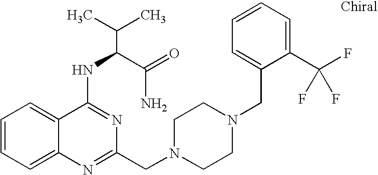 | 21-51 | 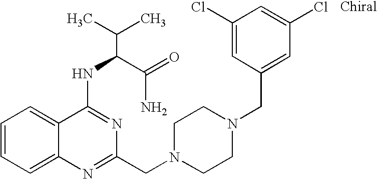 |
| 21-45 | 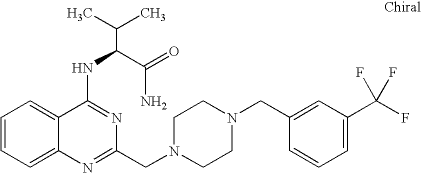 | 21-52 | 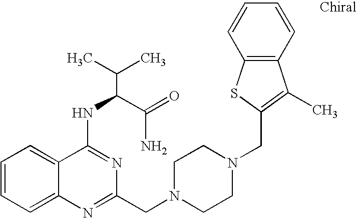 |
| 21-46 | 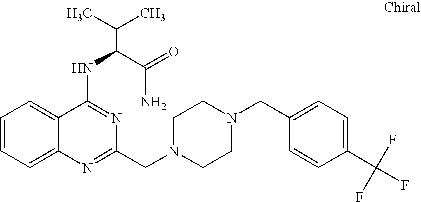 | 21-53 | 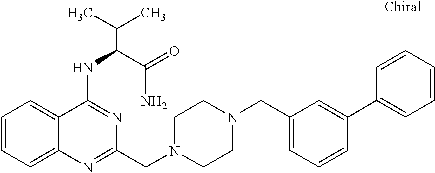 |
| 21-47 | 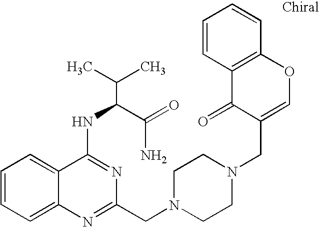 | 21-54 | 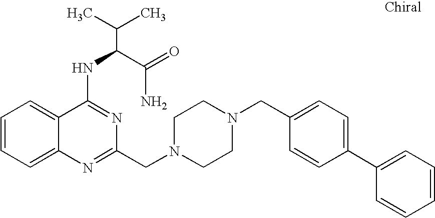 |
| 21-48 | 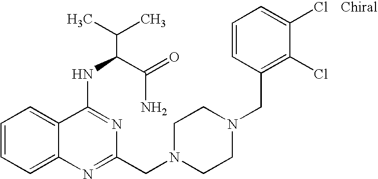 | 21-55 | 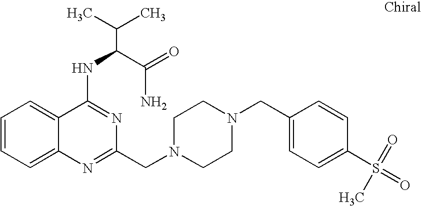 |
| 21-49 | 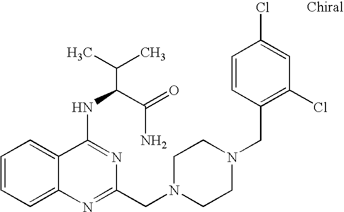 | 21-56 | 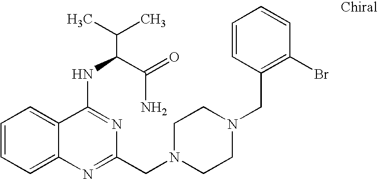 |
| 21-50 | 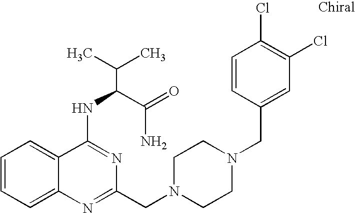 | 21-57 | 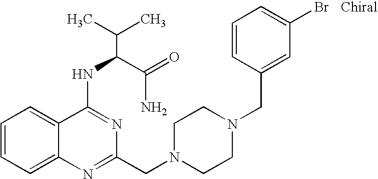 |
| 21-58 | 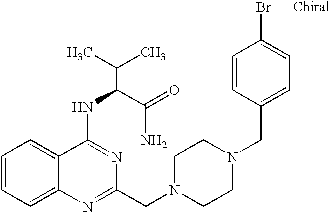 | 21-65 | 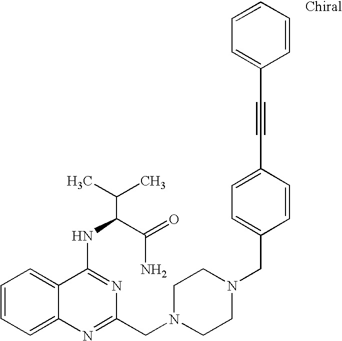 |
| 21-59 | 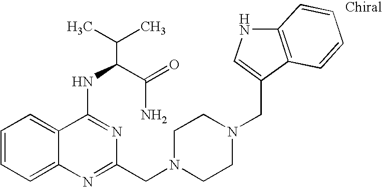 | 21-66 | 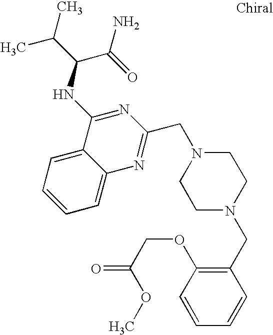 |
| 21-60 | 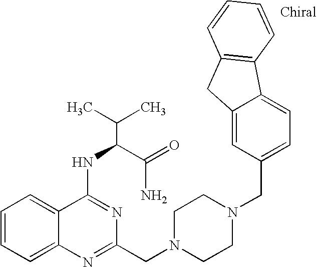 | 21-67 | 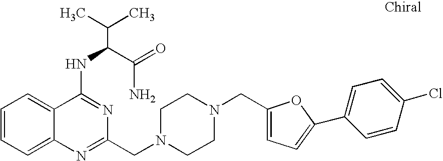 |
| 21-61 | 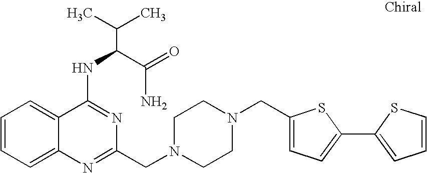 | 21-68 | 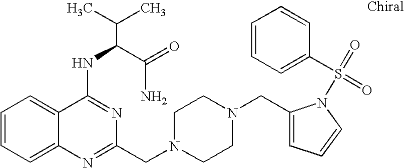 |
| 21-62 | 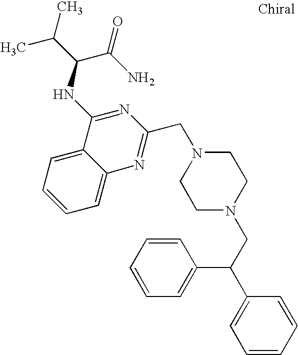 | 21-69 | 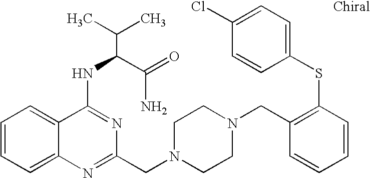 |
| 21-63 | 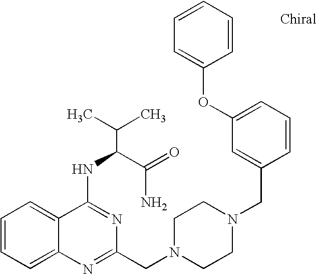 | 21-70 | 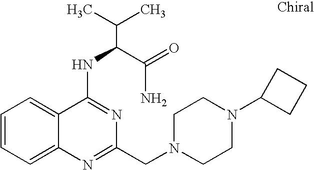 |
| 21-64 | 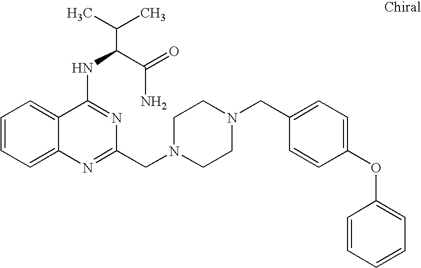 | 21-71 | 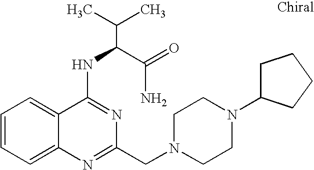 |
| 21-72 | 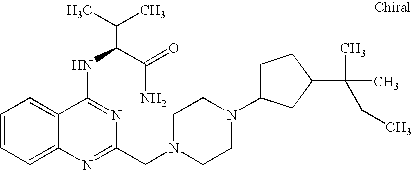 | 21-79 | 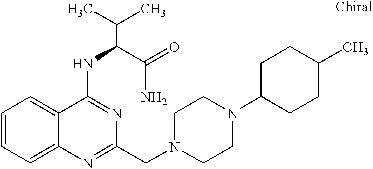 |
| 21-73 | 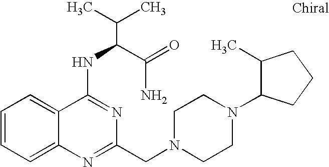 | 21-80 | 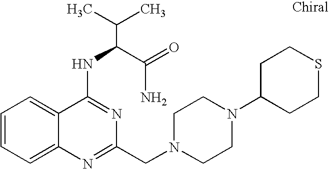 |
| 21-74 | 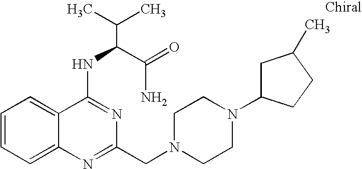 | 21-81 | 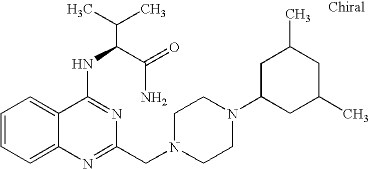 |
| 21-75 | 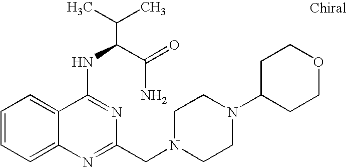 | 21-82 | 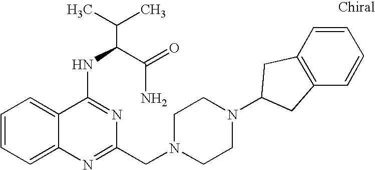 |
| 21-76 | 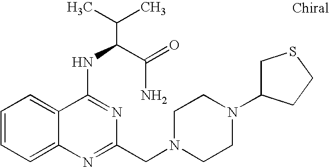 | 21-83 | 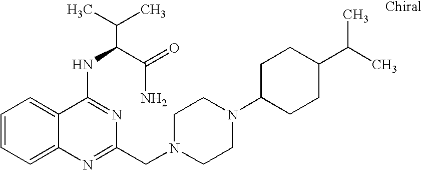 |
| 21-77 | 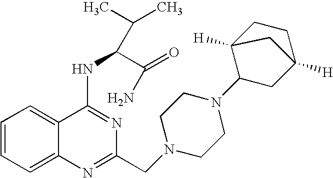 | 21-84 | 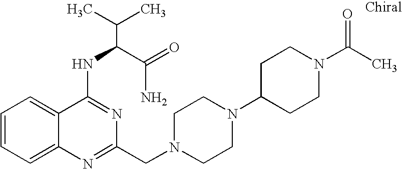 |
| 21-78 | 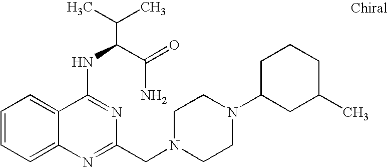 | 21-85 | 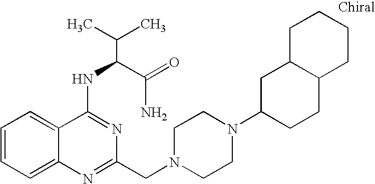 |
| 21-86 | 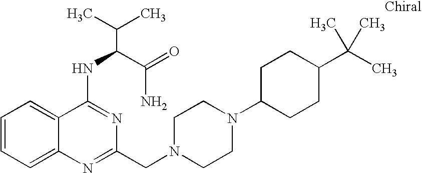 | 21-93 | 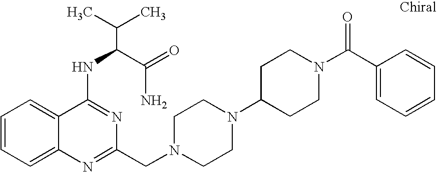 |
| 21-87 | 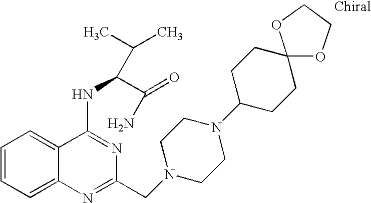 | 21-94 | 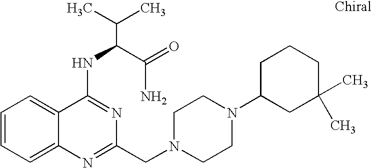 |
| 21-88 | 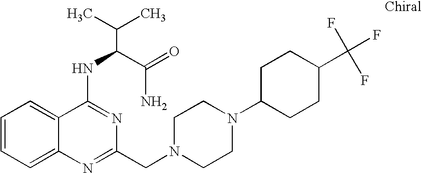 | 21-95 | 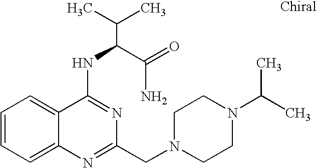 |
| 21-89 | 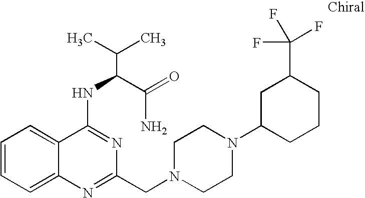 | 21-96 | 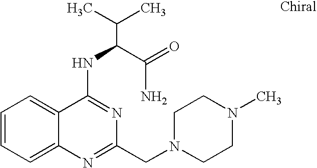 |
| 21-90 | 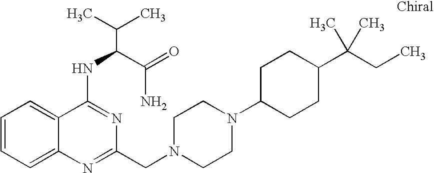 | 21-97 | 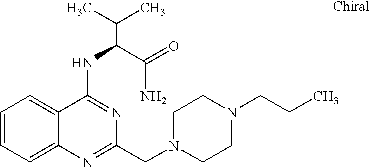 |
| 21-91 | 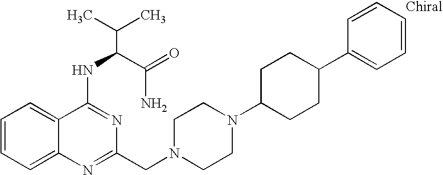 | 22-1 | 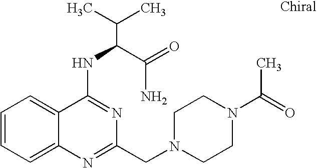 |
| 21-92 | 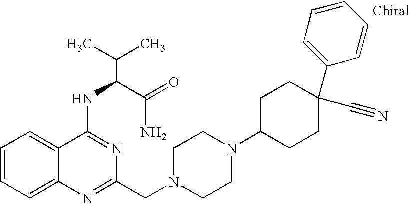 | 22-2 | 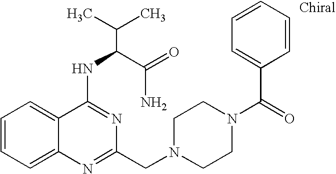 |
| 22-3 | 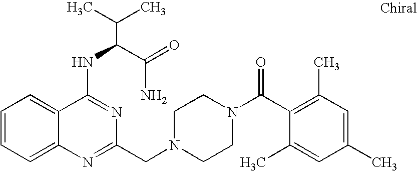 | 22-10 | 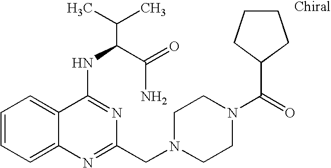 |
| 22-4 | 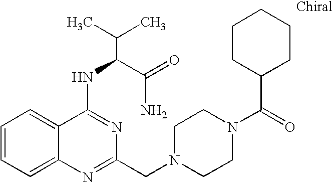 | 22-11 | 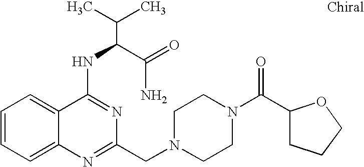 |
| 22-5 | 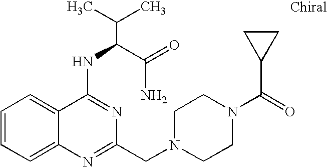 | 22-12 | 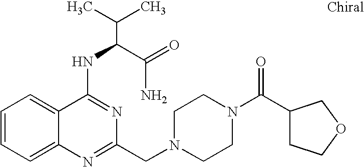 |
| 22-6 | 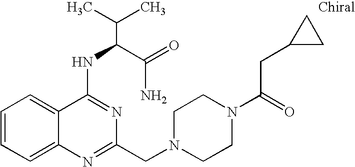 | 22-13 | 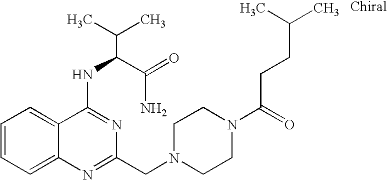 |
| 22-7 | 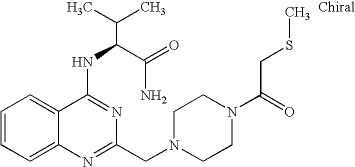 | 22-14 | 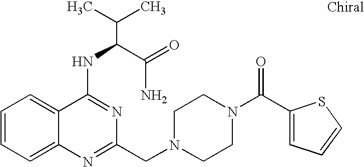 |
| 22-8 | 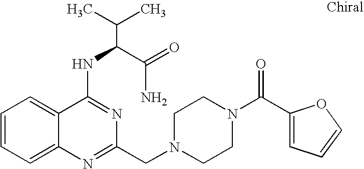 | 22-15 | 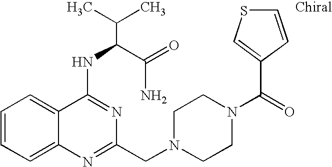 |
| 22-9 | 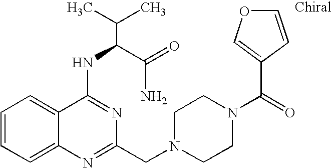 | 22-16 | 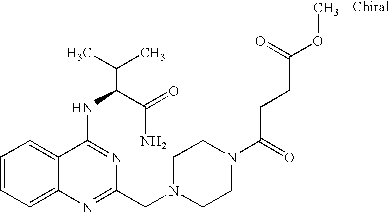 |
| 22-17 | 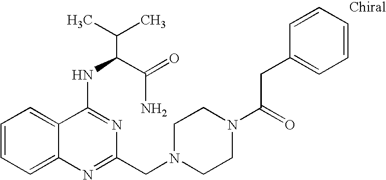 | 22-24 | 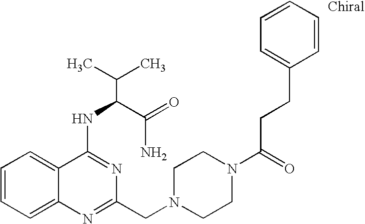 |
| 22-18 | 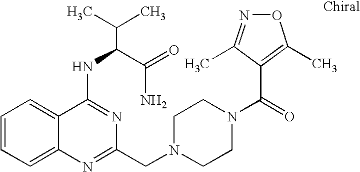 | 22-25 | 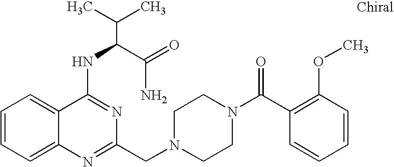 |
| 22-19 | 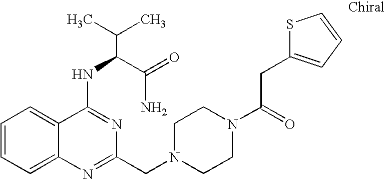 | 22-26 | 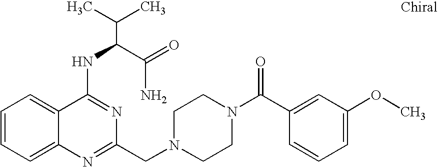 |
| 22-20 | 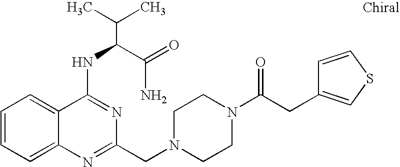 | 22-27 | 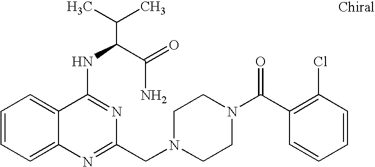 |
| 22-21 | 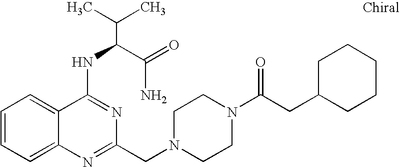 | 22-28 | 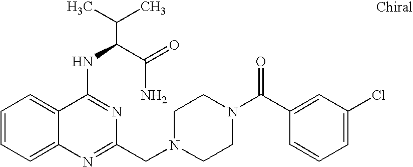 |
| 22-22 | 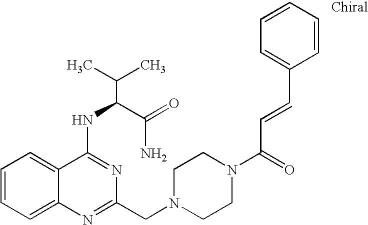 | 22-29 | 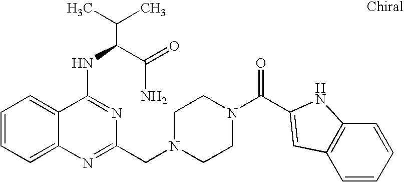 |
| 22-23 | 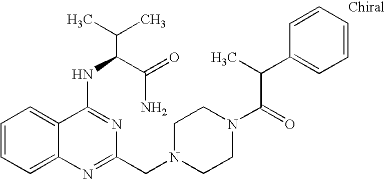 | 22-30 | 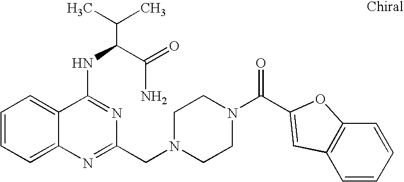 |
| 22-31 | 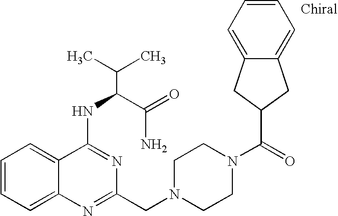 | 22-38 | 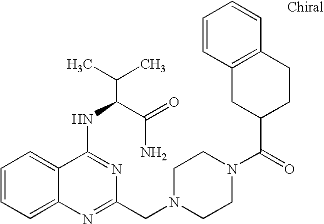 |
| 22-32 | 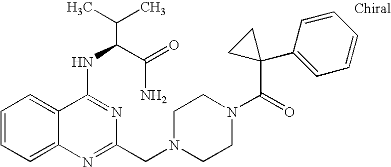 | 22-39 | 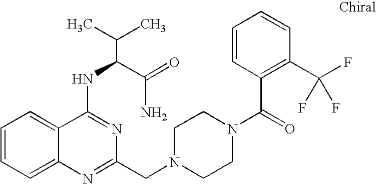 |
| 22-33 | 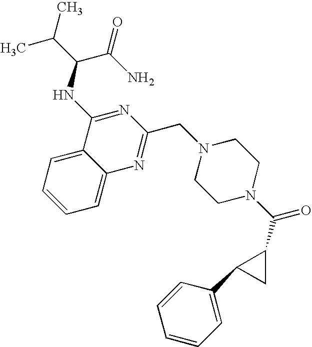 | 22-40 | 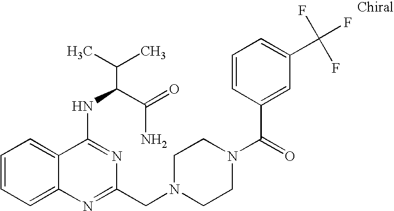 |
| 22-34 | 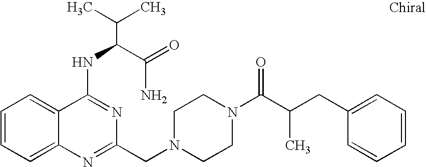 | 22-41 | 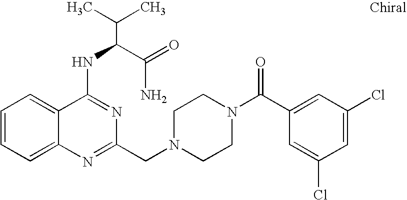 |
| 22-35 | 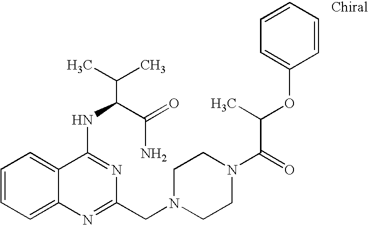 | 22-42 | 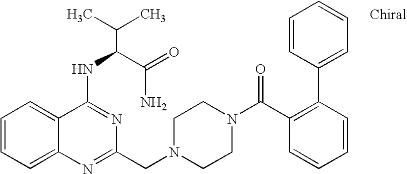 |
| 22-36 | 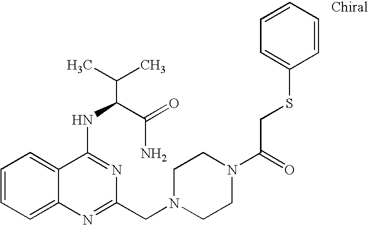 | 22-43 | 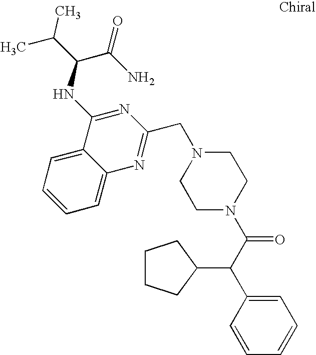 |
| 22-37 | 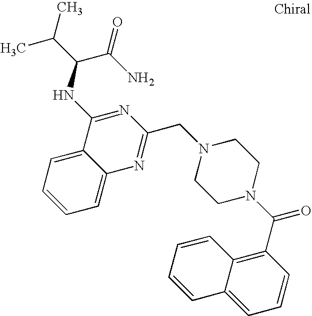 | 22-44 | 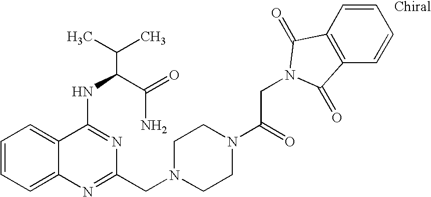 |
| 22-45 | 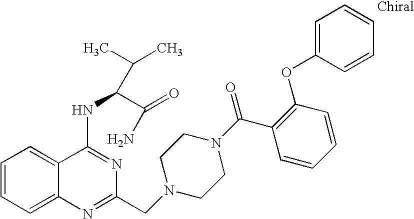 | 22-52 | 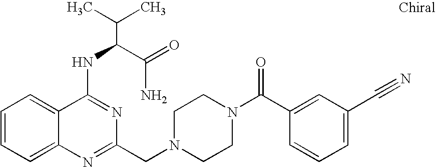 |
| 22-46 | 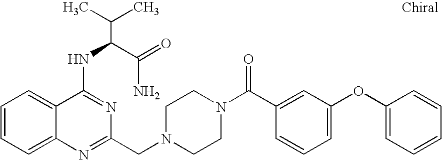 | 22-53 | 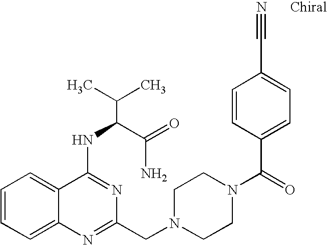 |
| 22-47 | 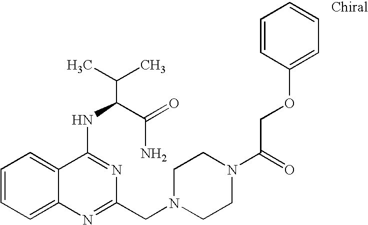 | 22-54 | 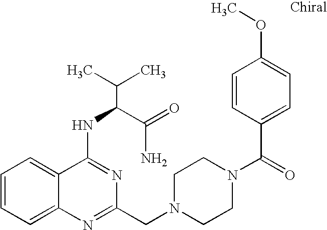 |
| 22-48 | 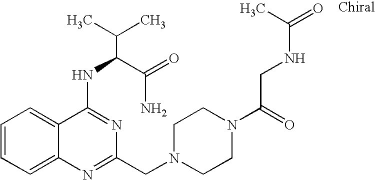 | 22-55 | 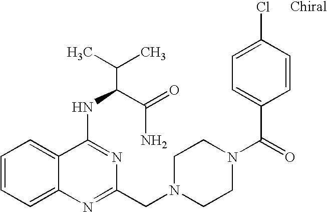 |
| 22-49 | 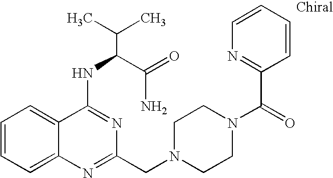 | 22-56 | 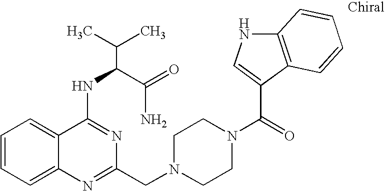 |
| 22-50 | 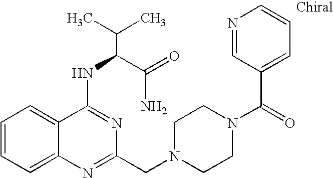 | 22-57 | 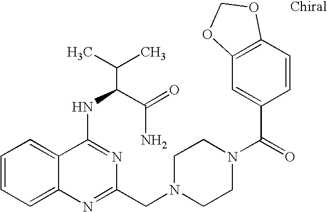 |
| 22-51 | 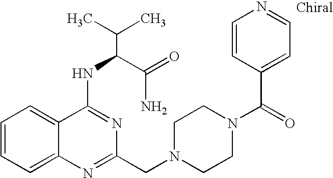 | 22-58 | 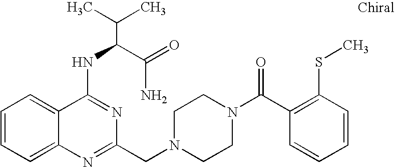 |
| 22-59 | 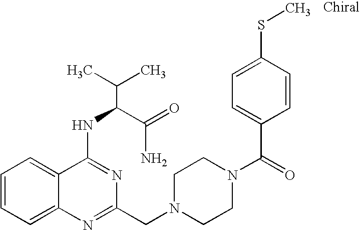 | 22-66 | 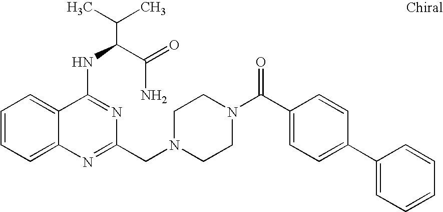 |
| 22-60 | 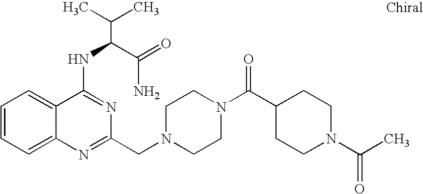 | 22-67 | 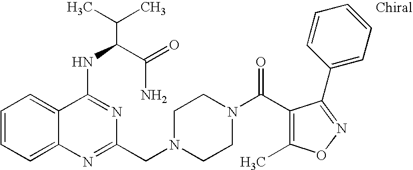 |
| 22-61 | 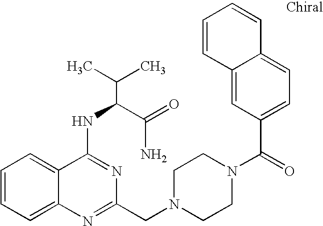 | 22-68 | 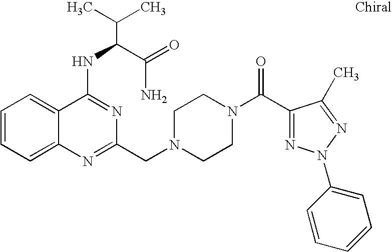 |
| 22-62 | 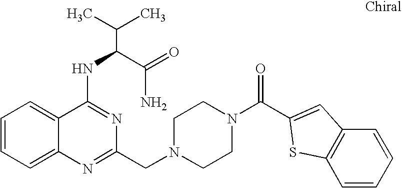 | 22-69 | 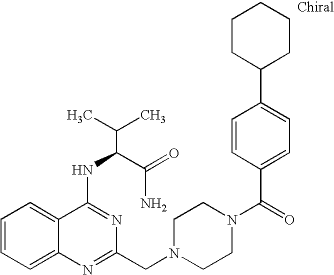 |
| 22-63 | 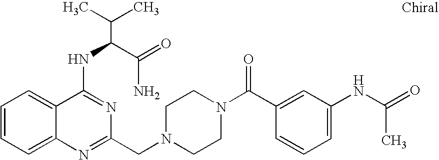 | 22-70 | 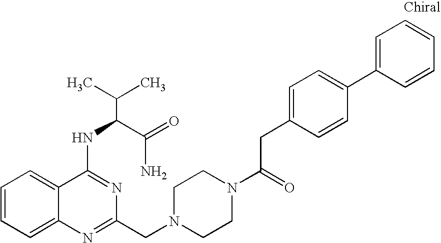 |
| 22-64 | 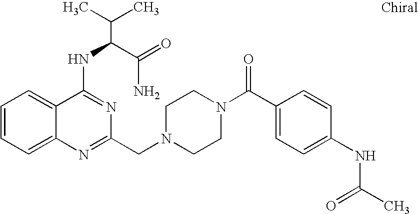 | 22-71 | 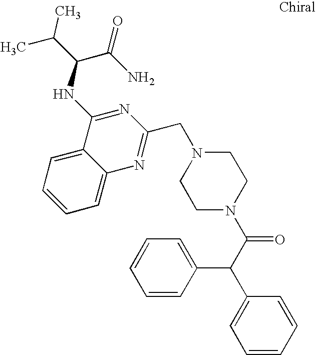 |
| 22-65 | 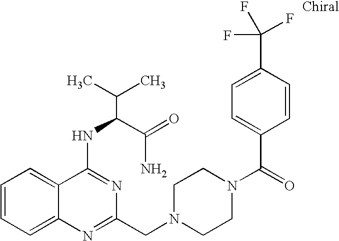 | 23-1 | 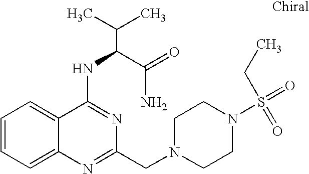 |
| 23-2 | 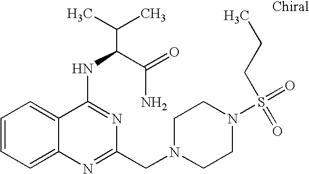 | 23-9 | 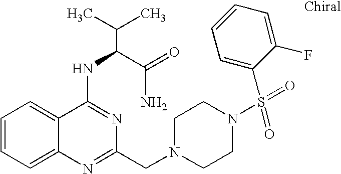 |
| 23-3 | 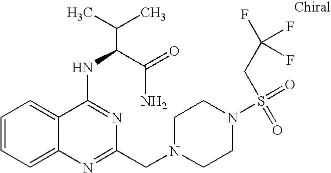 | 23-10 | 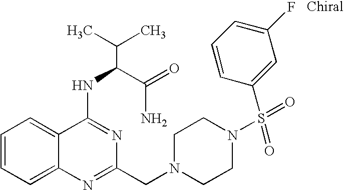 |
| 23-4 | 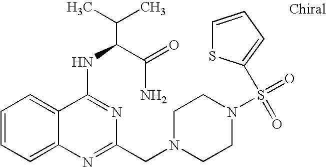 | 23-11 | 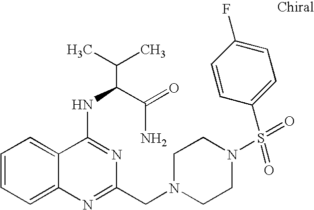 |
| 23-5 | 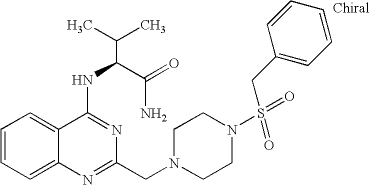 | 23-12 | 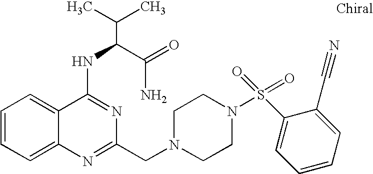 |
| 23-6 | 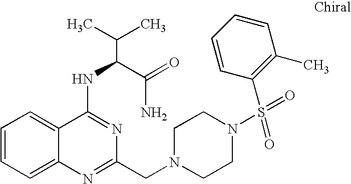 | 23-13 | 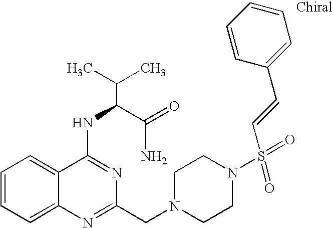 |
| 23-7 | 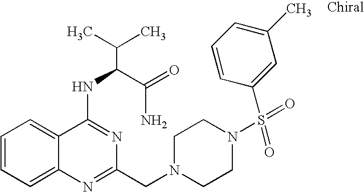 | 23-14 | 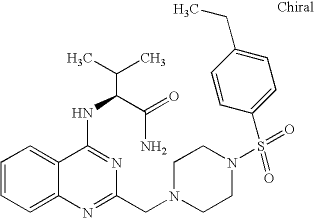 |
| 23-8 | 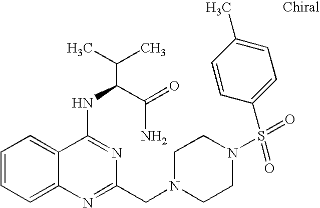 | 23-15 | 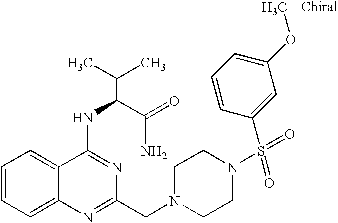 |
| 23-16 | 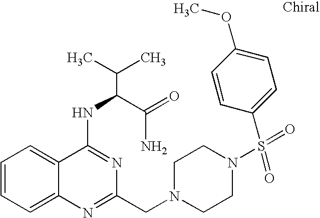 | 23-23 | 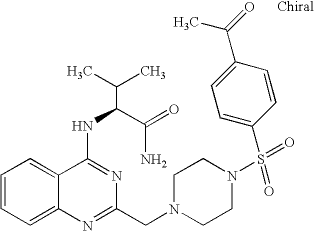 |
| 23-17 | 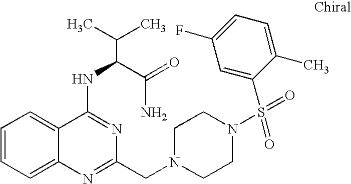 | 23-24 | 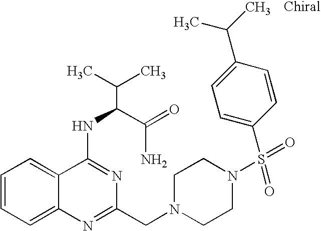 |
| 23-18 | 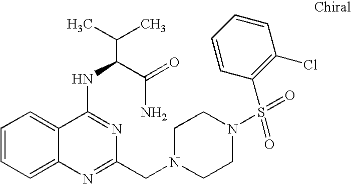 | 23-25 | 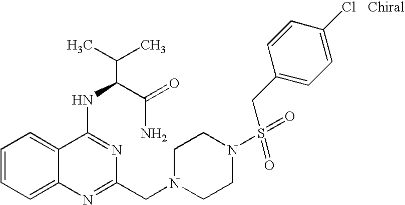 |
| 23-19 | 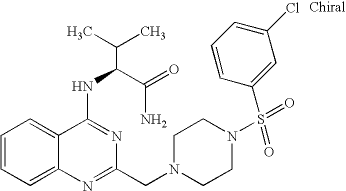 | 23-26 | 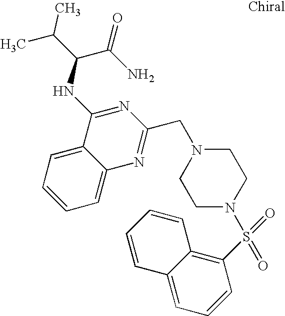 |
| 23-20 | 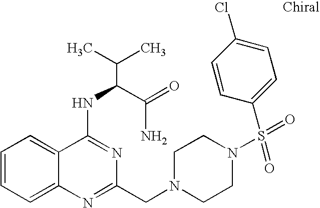 | 23-27 | 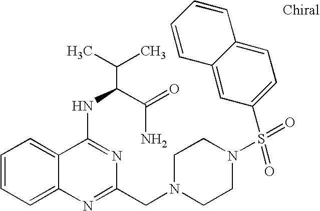 |
| 23-21 | 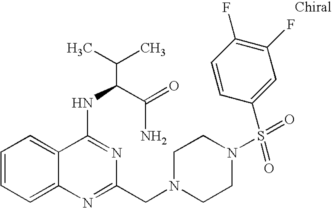 | 23-28 | 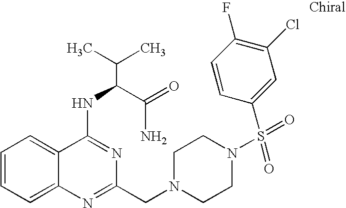 |
| 23-22 | 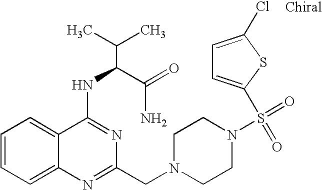 | 23-29 | 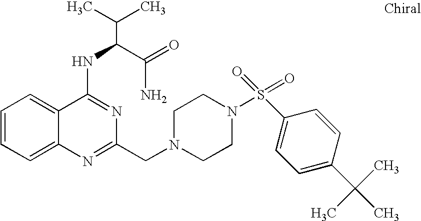 |
| 23-30 | 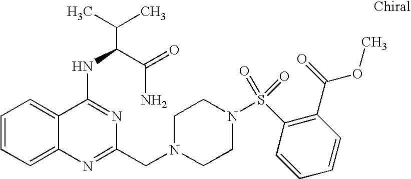 | 23-37 | 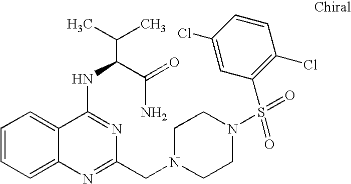 |
| 23-31 | 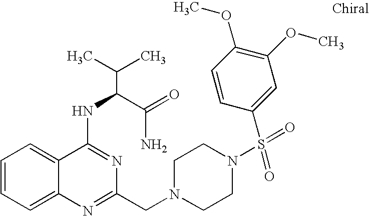 | 23-38 | 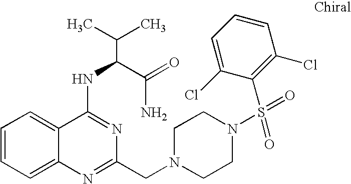 |
| 23-32 | 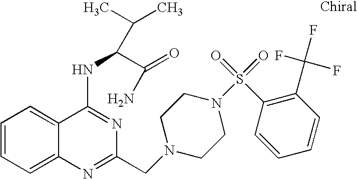 | 23-39 | 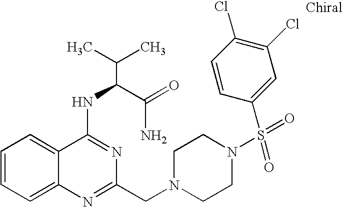 |
| 23-33 | 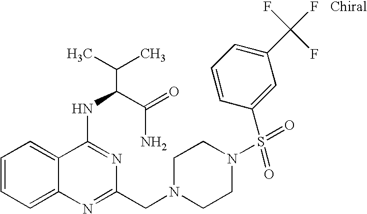 | 23-40 | 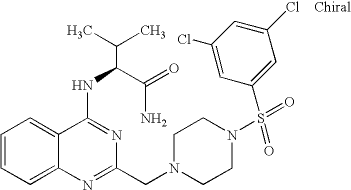 |
| 23-34 | 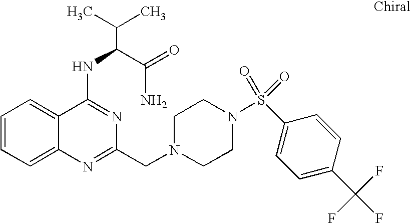 | 23-41 | 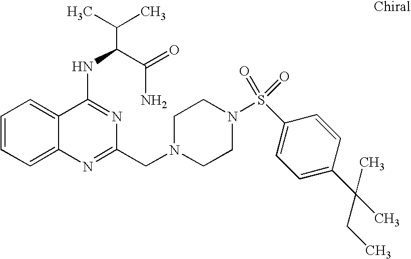 |
| 23-35 | 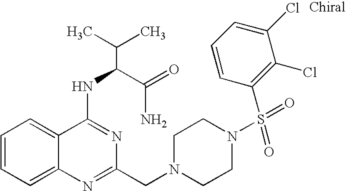 | 23-42 | 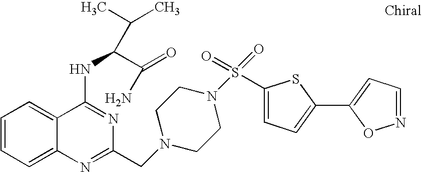 |
| 23-36 | 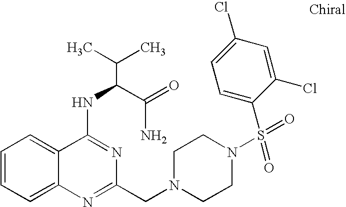 | 23-43 | 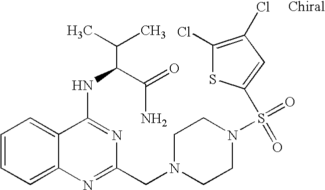 |
| 23-44 | 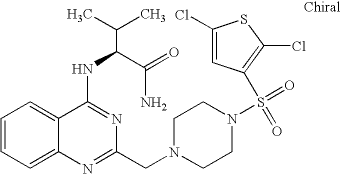 | 23-51 | 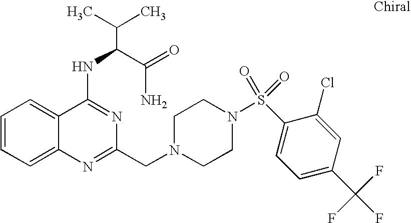 |
| 23-45 | 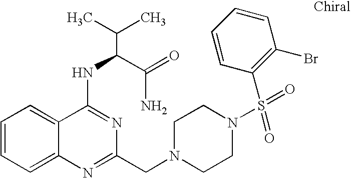 | 23-52 | 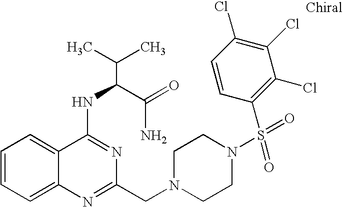 |
| 23-46 | 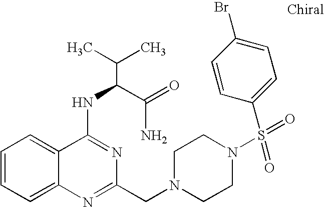 | 23-53 | 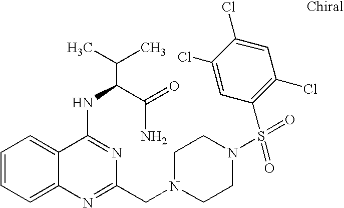 |
| 23-47 | 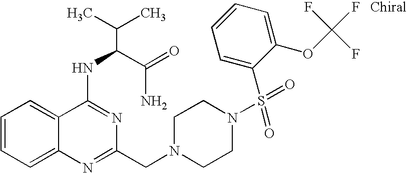 | 23-54 | 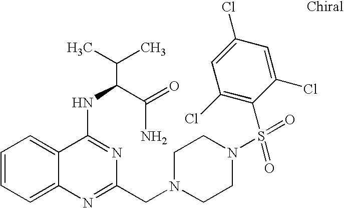 |
| 23-48 | 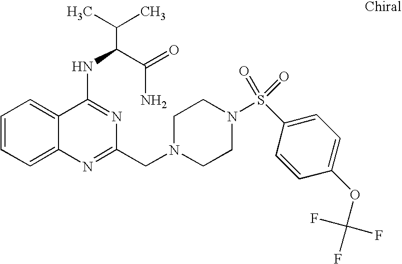 | 23-55 | 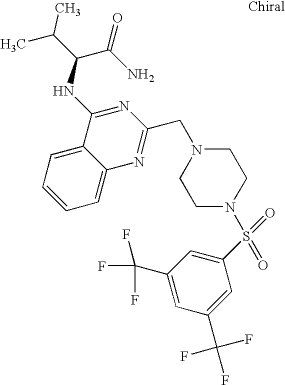 |
| 23-49 | 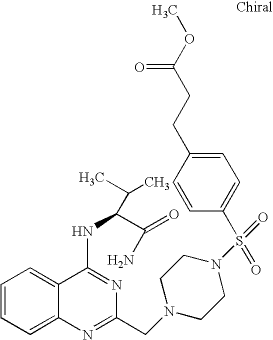 | 23-56 | 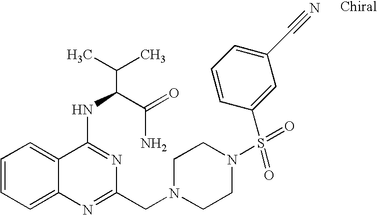 |
| 23-50 | 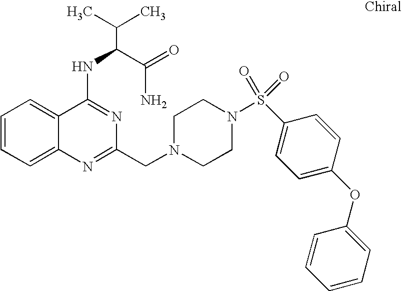 | 23-57 | 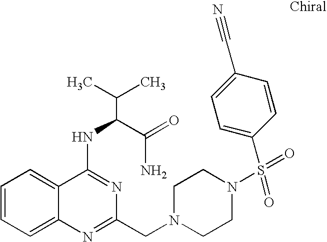 |
| 23-58 | 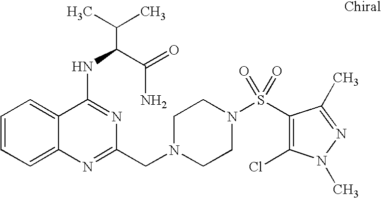 | 23-65 | 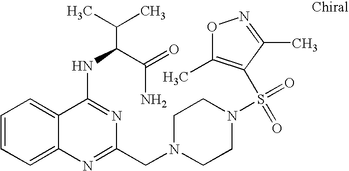 |
| 23-59 | 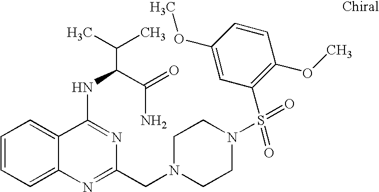 | 23-66 | 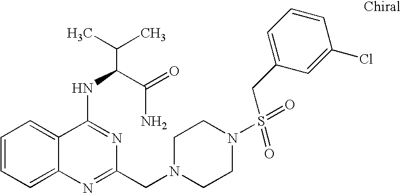 |
| 23-60 | 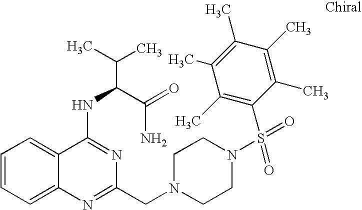 | 23-67 | 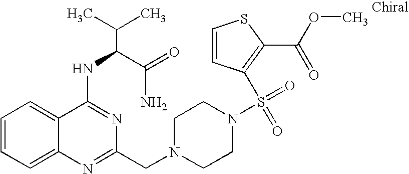 |
| 23-61 | 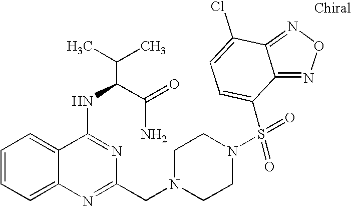 | 23-68 | 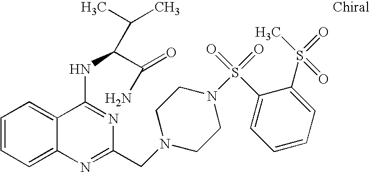 |
| 23-62 | 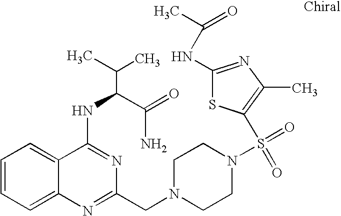 | 23-69 | 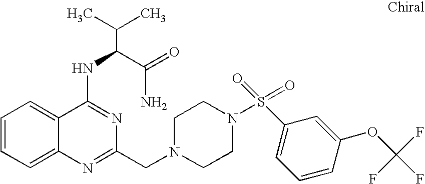 |
| 23-63 | 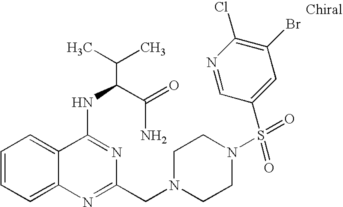 | 23-70 | 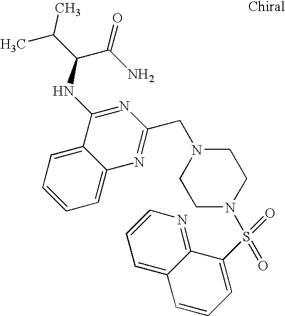 |
| 23-64 | 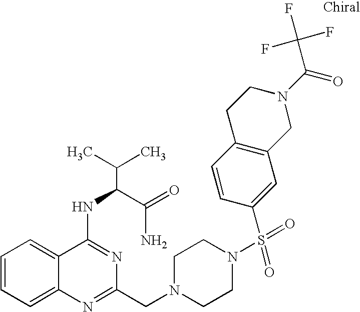 | 23-71 | 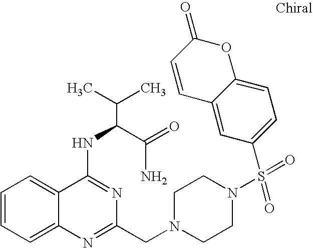 |
| 23-72 | 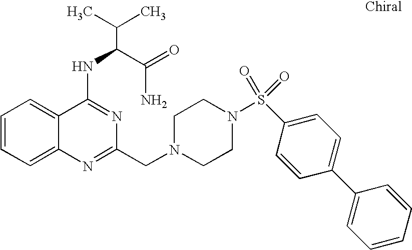 | 24-2 | 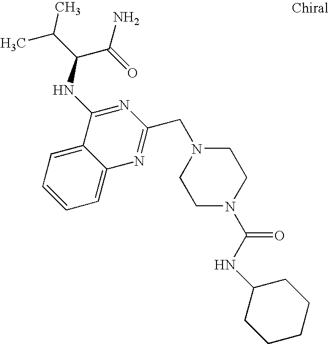 |
| 23-73 | 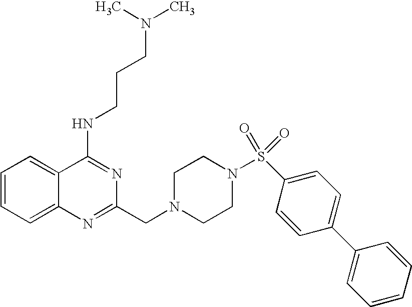 | 24-3 | 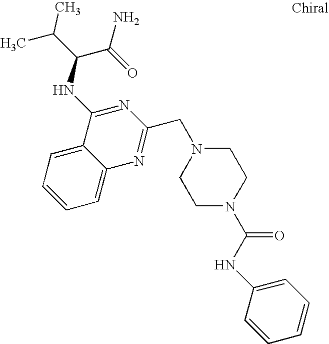 |
| 23-74 | 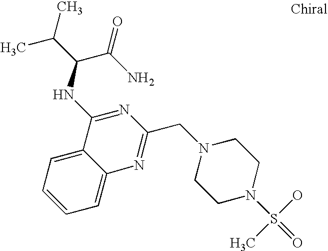 | 24-4 | 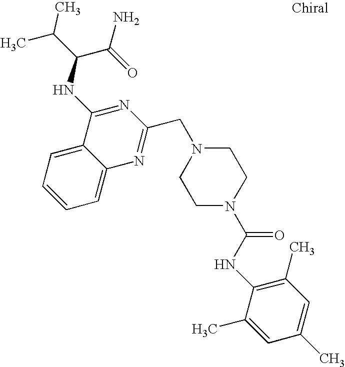 |
| 23-75 | 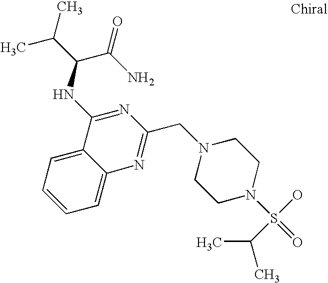 | 24-5 | 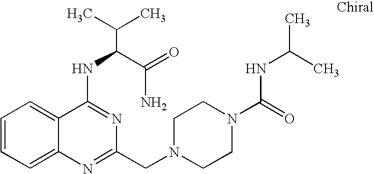 |
| 23-76 | 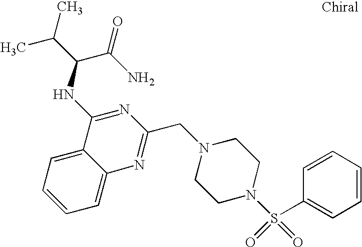 | 24-6 | 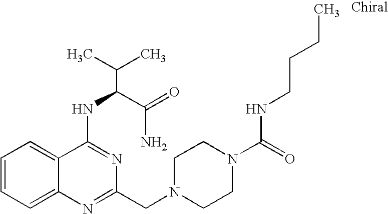 |
| 23-77 | 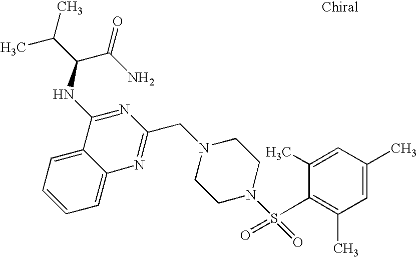 | 24-7 | 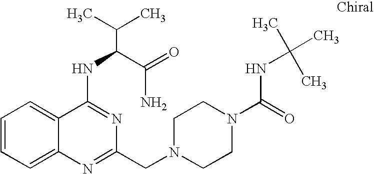 |
| 24-1 | 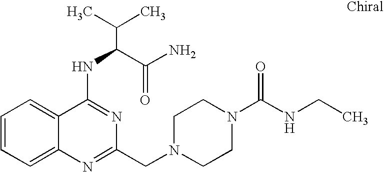 | 24-8 | 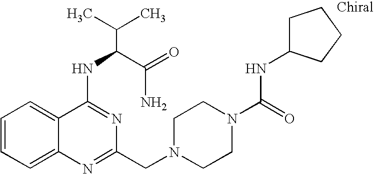 |
| 24-9 | 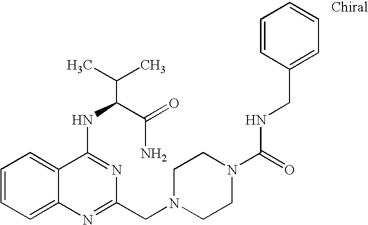 | 24-16 | 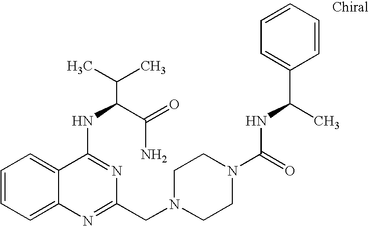 |
| 24-10 | 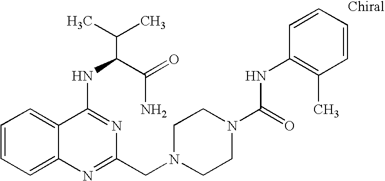 | 24-17 | 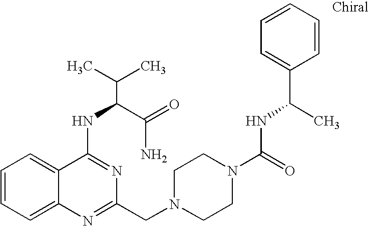 |
| 24-11 | 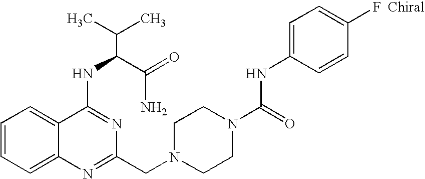 | 24-18 | 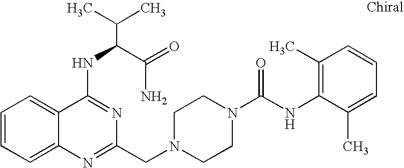 |
| 24-12 | 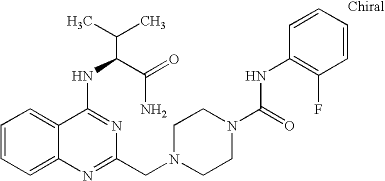 | 24-19 | 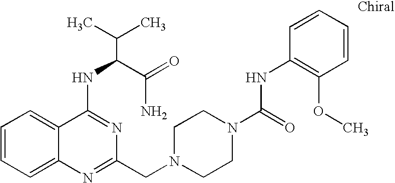 |
| 24-13 | 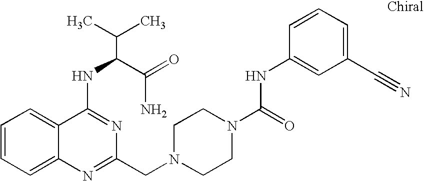 | 24-20 | 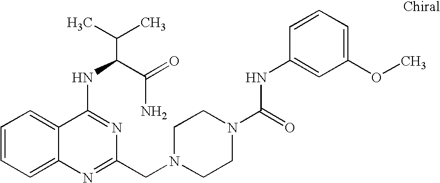 |
| 24-14 | 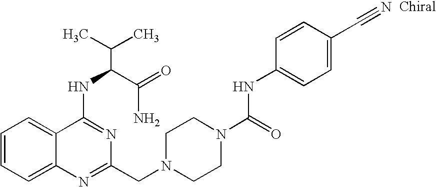 | 24-21 | 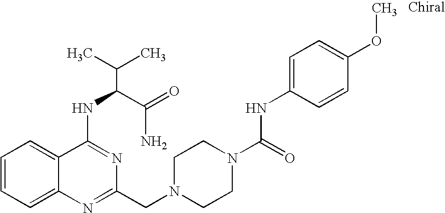 |
| 24-15 | 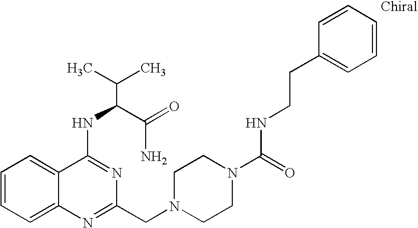 | 24-22 | 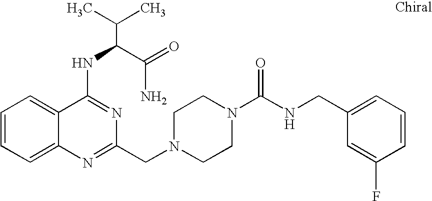 |
| 24-23 | 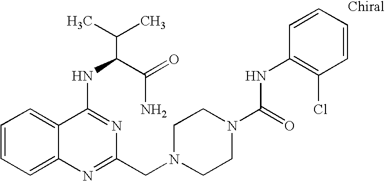 | 24-30 | 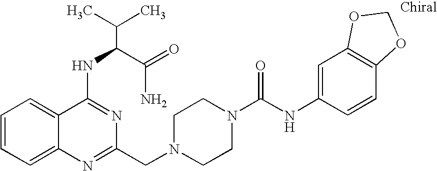 |
| 24-24 | 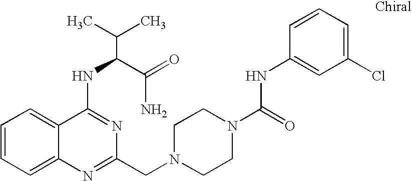 | 24-31 | 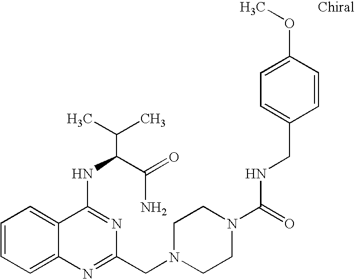 |
| 24-25 | 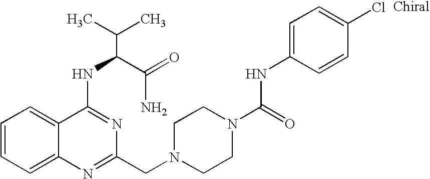 | 24-32 | 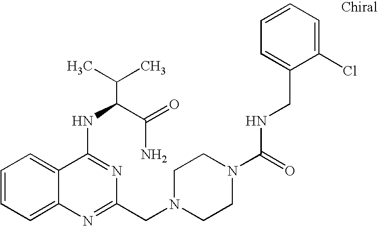 |
| 24-26 | 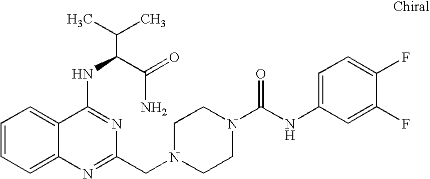 | 24-33 | 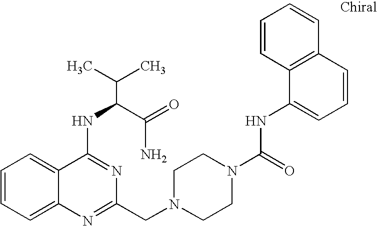 |
| 24-27 | 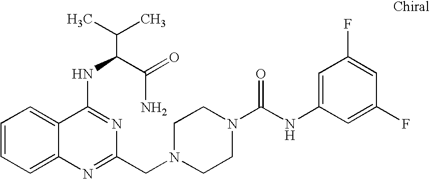 | 24-34 | 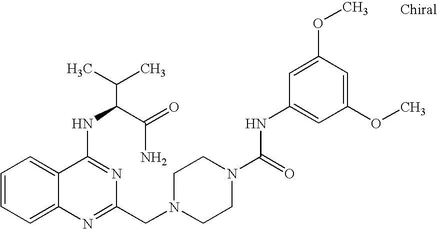 |
| 24-28 | 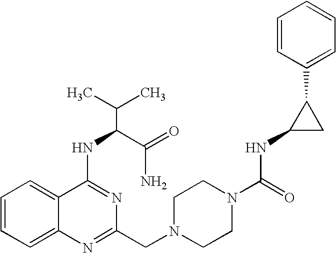 | 24-35 | 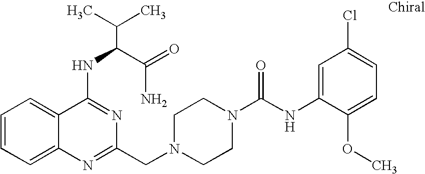 |
| 24-29 | 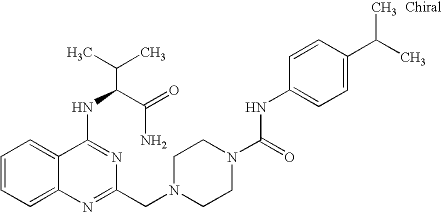 | 24-36 | 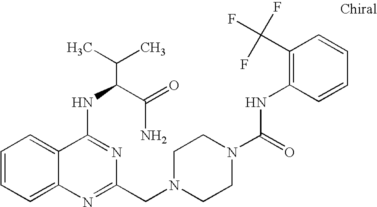 |
| 24-37 | 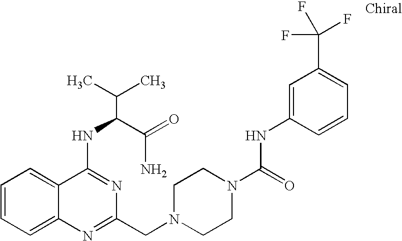 | 24-44 | 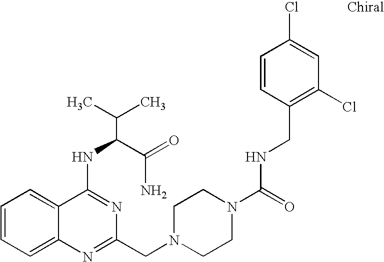 |
| 24-38 | 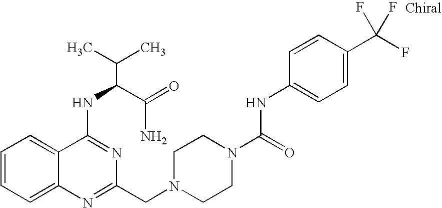 | 24-45 | 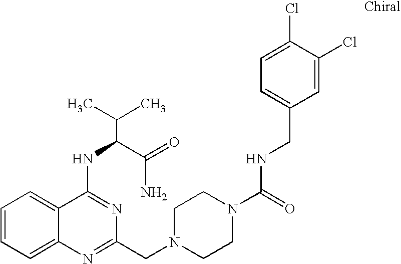 |
| 24-39 | 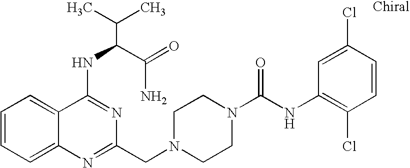 | 24-46 | 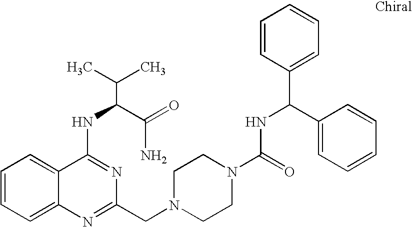 |
| 24-40 | 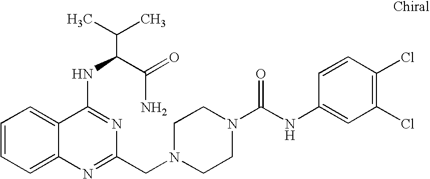 | 24-47 | 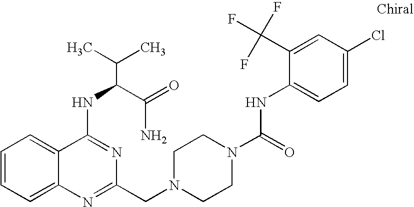 |
| 24-41 | 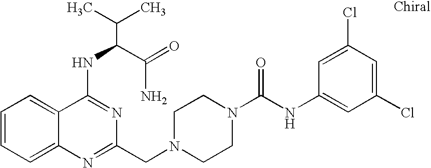 | 24-48 | 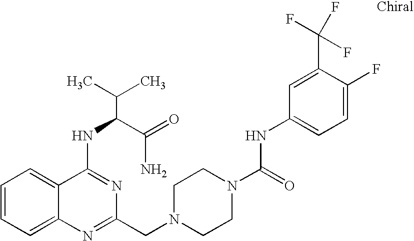 |
| 24-42 | 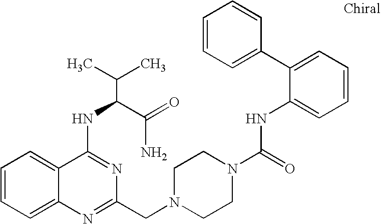 | 24-49 | 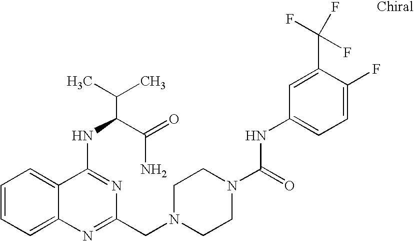 |
| 24-43 | 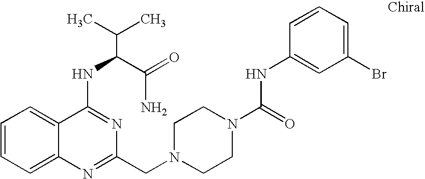 | 25-1 | 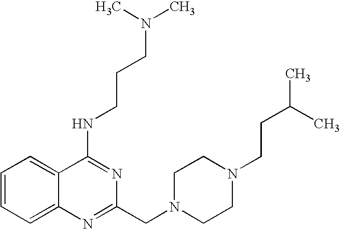 |
| 25-2 | 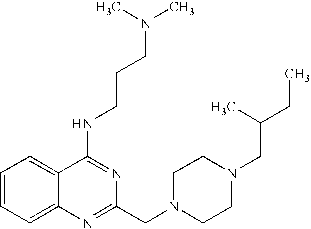 | 25-9 | 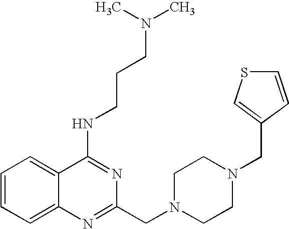 |
| 25-3 | 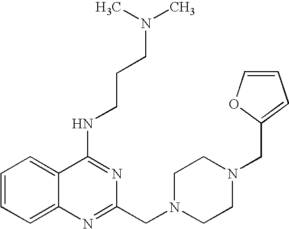 | 25-10 | 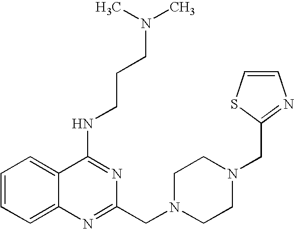 |
| 25-4 | 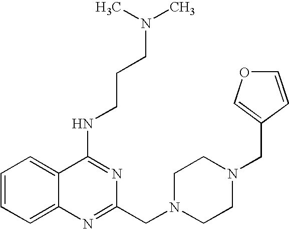 | 25-11 | 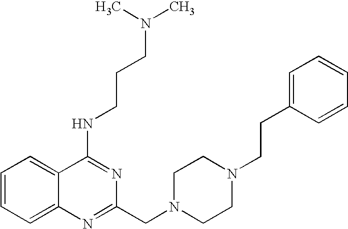 |
| 25-5 | 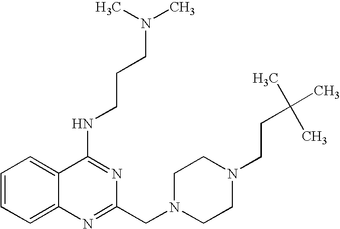 | 25-12 | 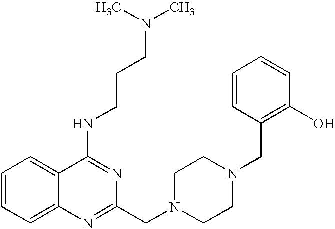 |
| 25-6 | 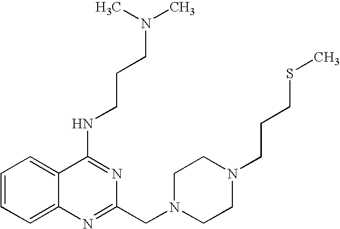 | 25-13 | 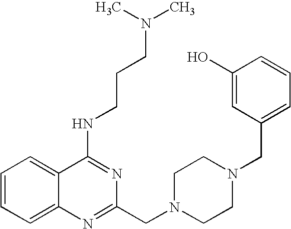 |
| 25-7 | 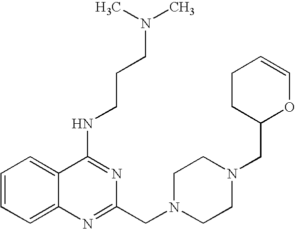 | 25-14 | 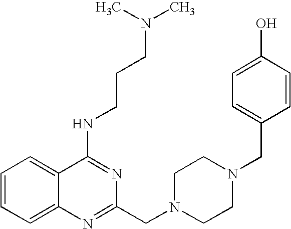 |
| 25-8 | 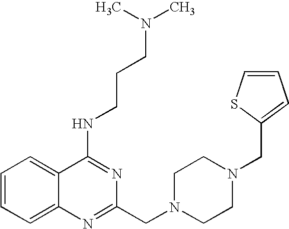 | 25-15 | 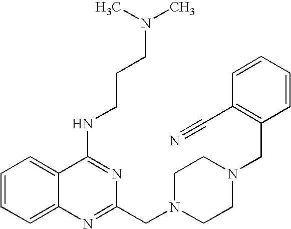 |
| 25-16 | 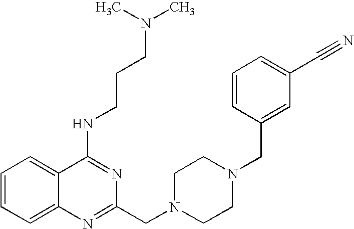 | 25-23 | 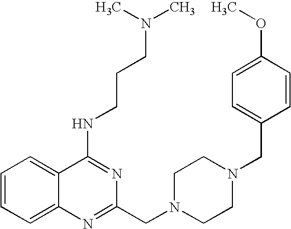 |
| 25-17 | 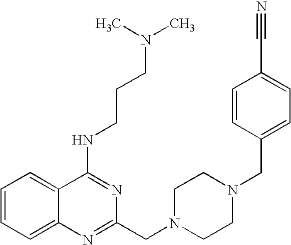 | 25-24 | 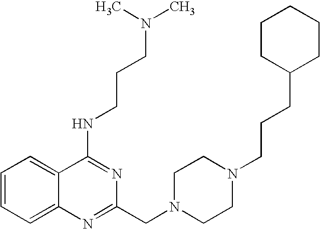 |
| 25-18 | 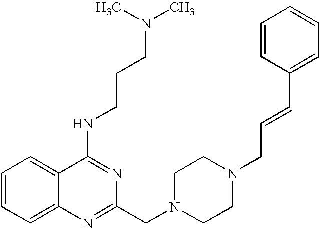 | 25-25 | 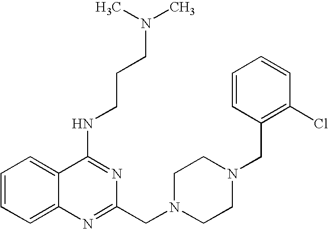 |
| 25-19 | 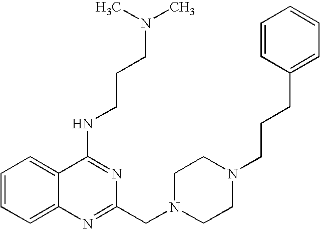 | 25-26 | 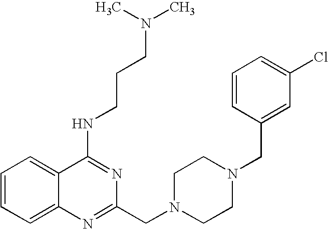 |
| 25-20 | 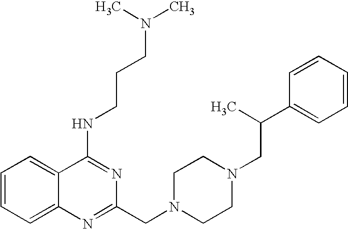 | 25-27 | 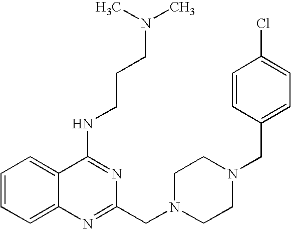 |
| 25-21 | 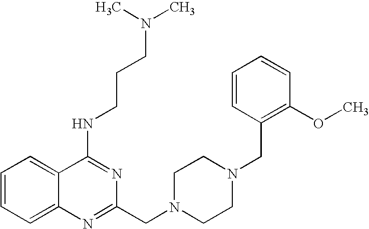 | 25-28 | 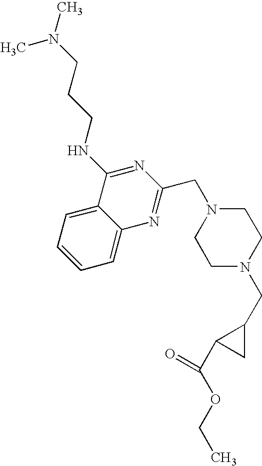 |
| 25-22 | 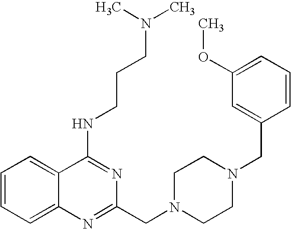 | 25-29 | 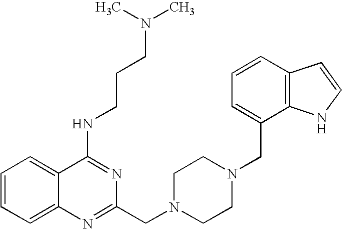 |
| 25-30 | 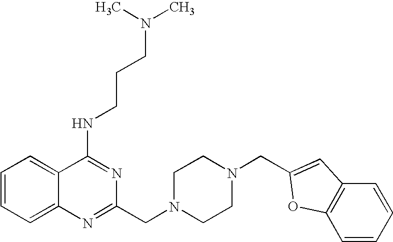 | 25-37 | 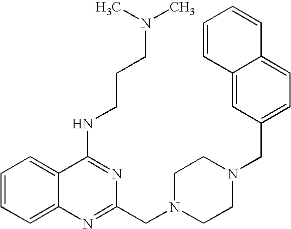 |
| 25-31 | 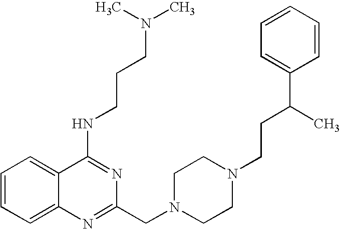 | 25-38 | 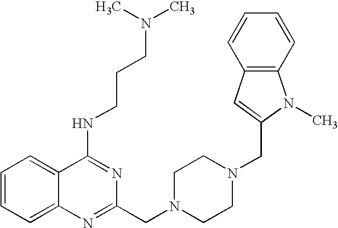 |
| 25-32 | 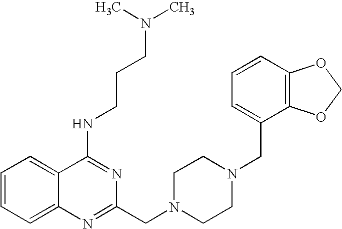 | 25-39 | 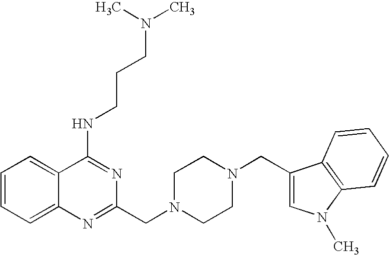 |
| 25-33 | 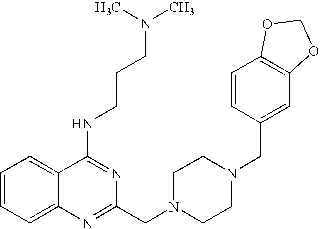 | 25-40 | 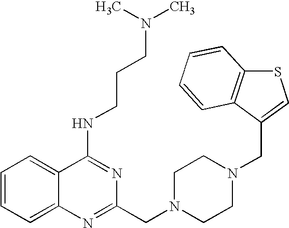 |
| 25-34 | 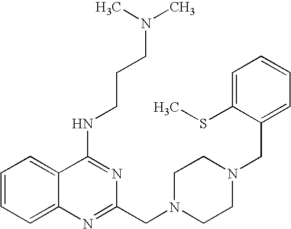 | 25-41 | 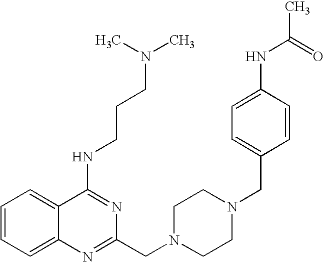 |
| 25-35 | 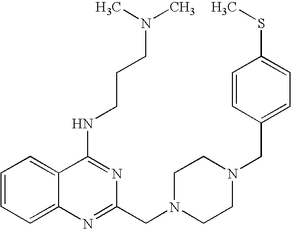 | 25-42 | 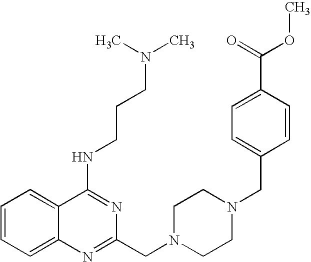 |
| 25-36 | 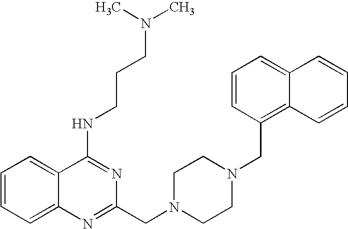 | 25-43 | 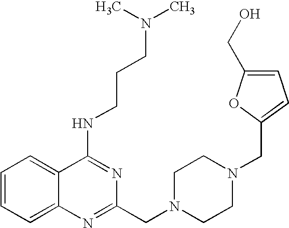 |
| 25-44 | 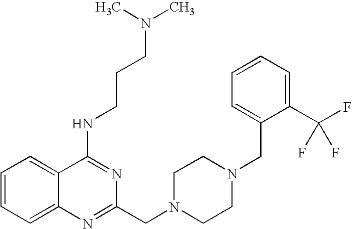 | 25-51 | 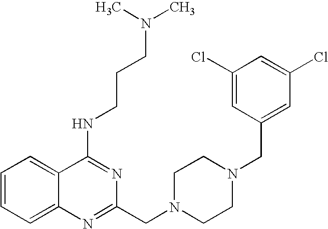 |
| 25-45 | 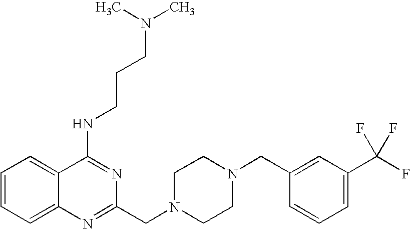 | 25-52 | 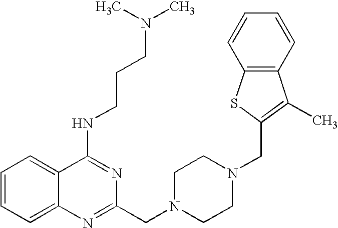 |
| 25-46 | 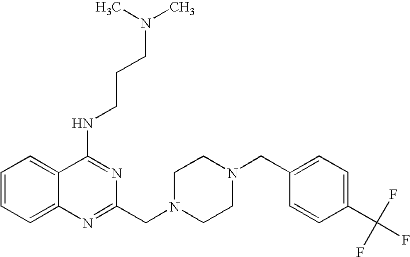 | 25-53 | 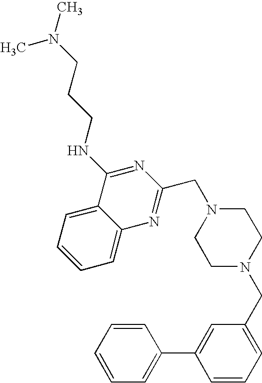 |
| 25-47 | 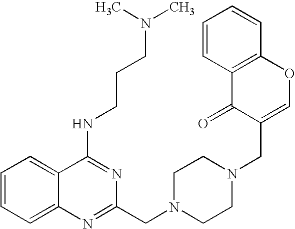 | 25-54 | 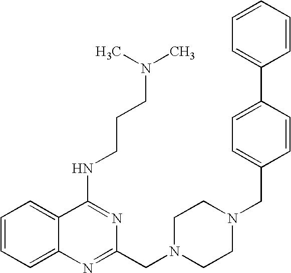 |
| 25-48 | 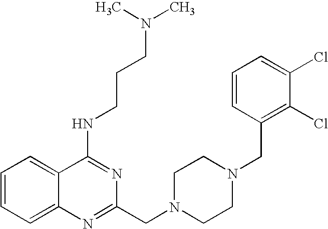 | 25-55 | 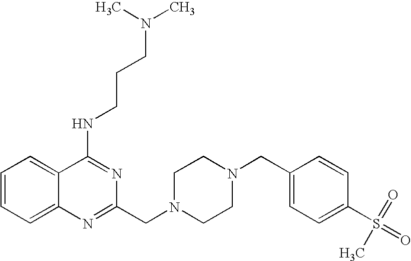 |
| 25-49 | 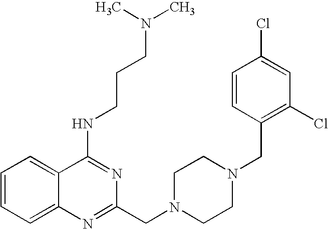 | 25-56 | 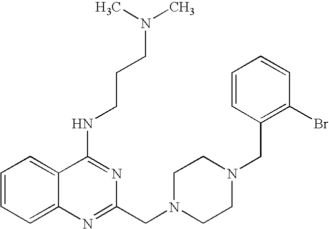 |
| 25-50 | 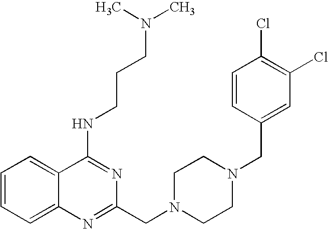 | 25-57 | 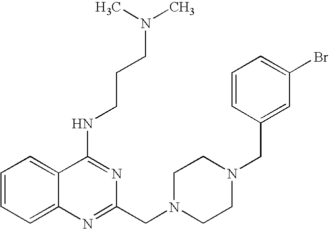 |
| 25-58 | 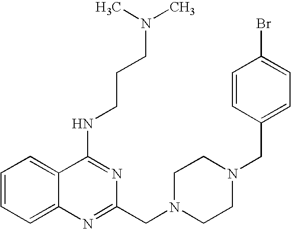 | 25-65 | 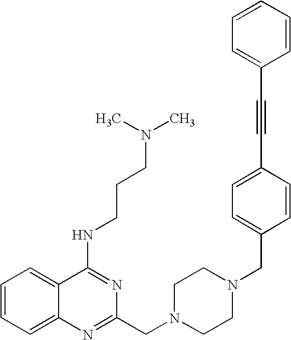 |
| 25-59 | 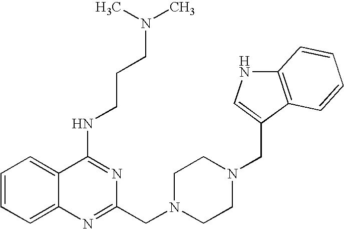 | 25-66 | 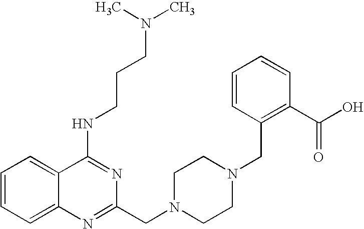 |
| 25-60 | 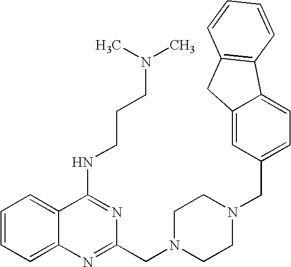 | 25-67 | 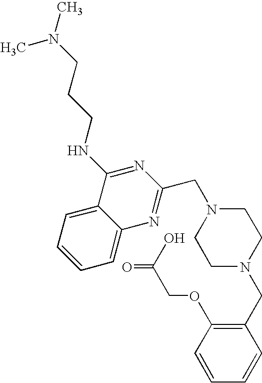 |
| 25-61 | 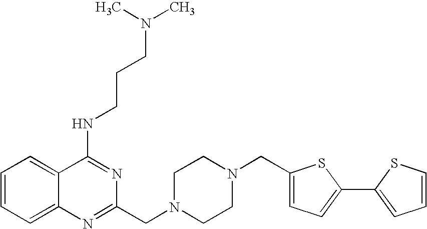 | 25-68 | 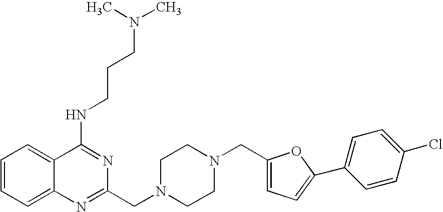 |
| 25-62 | 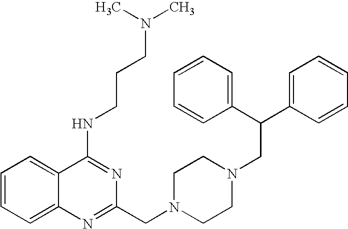 | 25-69 | 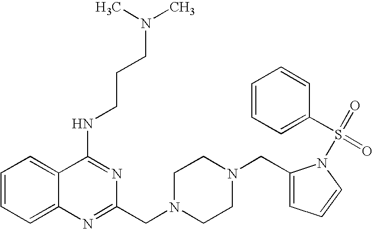 |
| 25-63 | 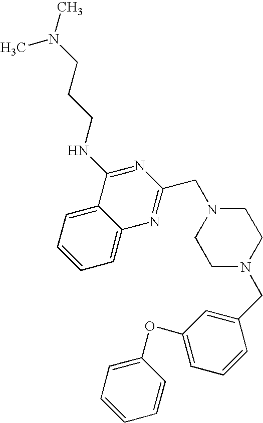 | 25-70 | 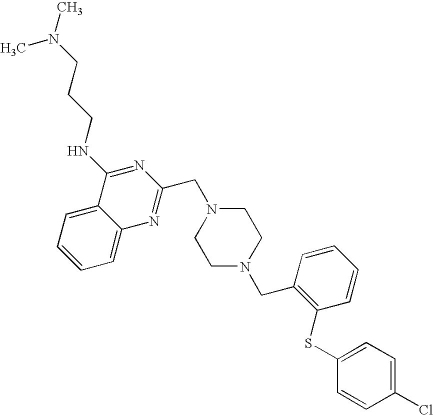 |
| 25-64 | 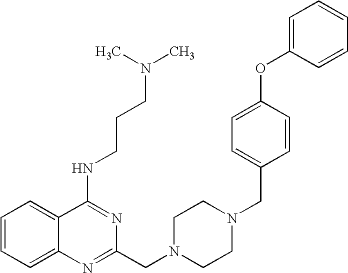 | 25-71 | 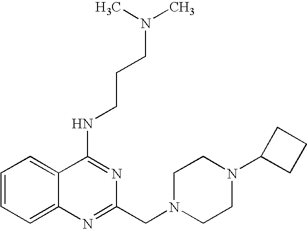 |
| 25-72 | 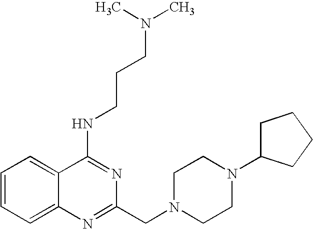 | 25-79 | 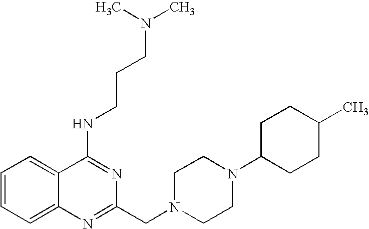 |
| 25-73 | 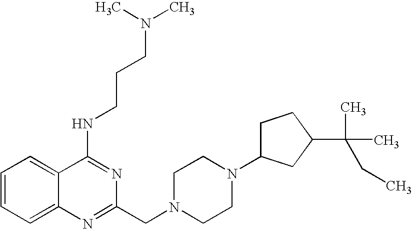 | 25-80 | 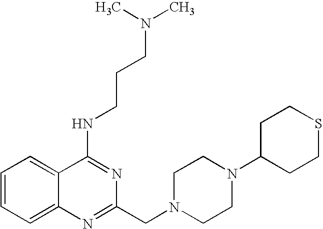 |
| 25-74 | 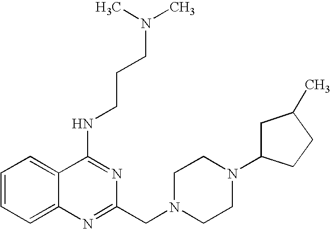 | 25-81 | 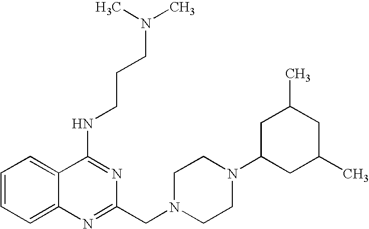 |
| 25-75 | 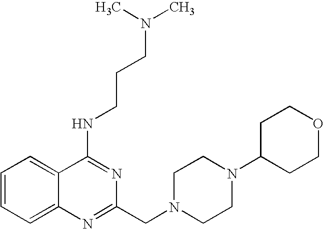 | 25-82 | 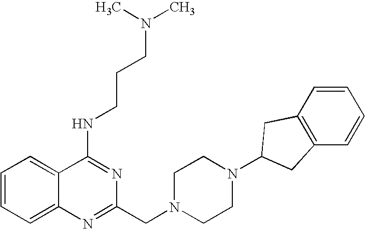 |
| 25-76 | 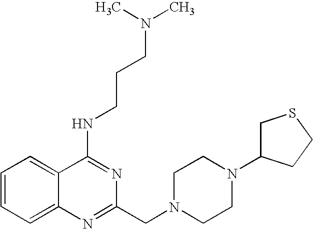 | 25-83 | 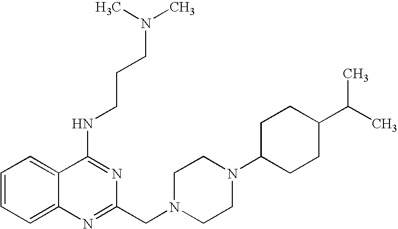 |
| 25-77 | 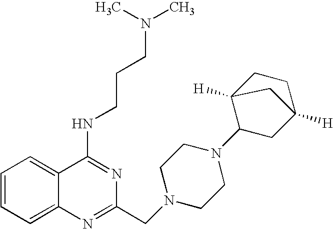 | 25-84 | 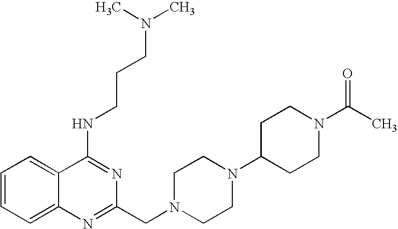 |
| 25-78 | 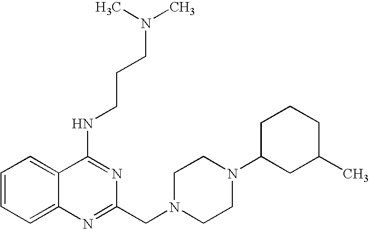 | 25-85 | 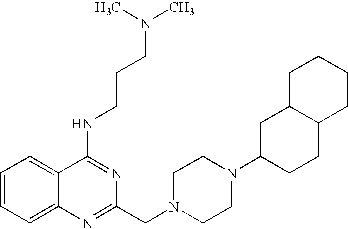 |
| 25-86 | 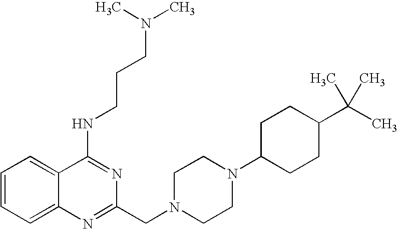 | 25-93 | 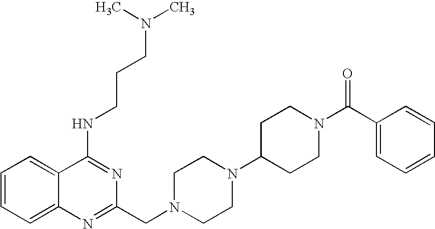 |
| 25-87 | 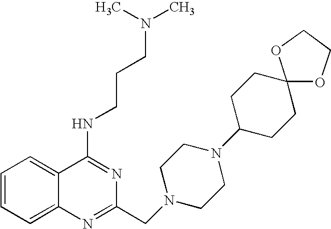 | 25-94 | 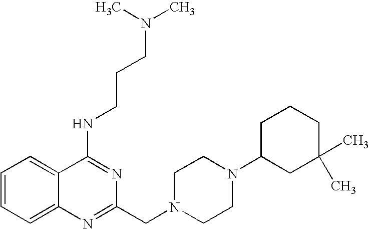 |
| 25-88 | 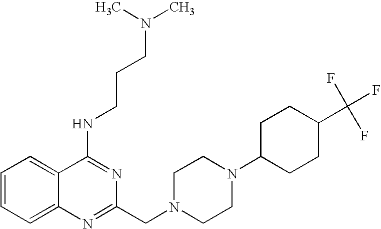 | 26-1 | 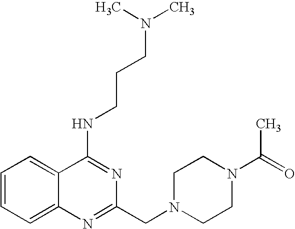 |
| 25-89 | 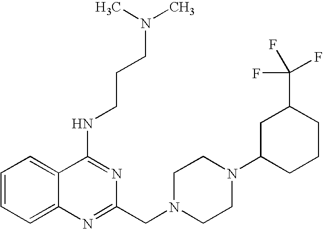 | 26-2 | 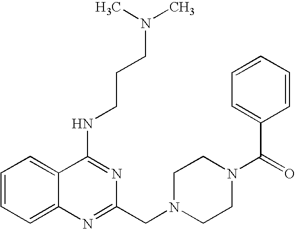 |
| 25-90 | 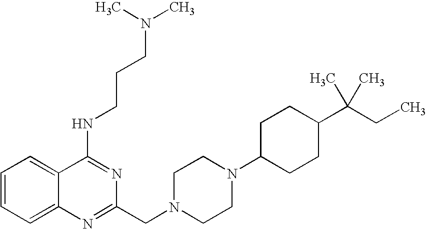 | 26-3 | 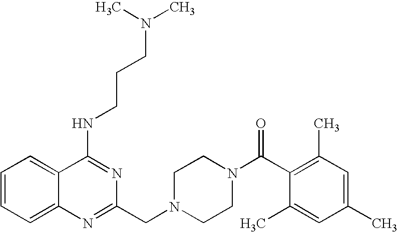 |
| 25-91 | 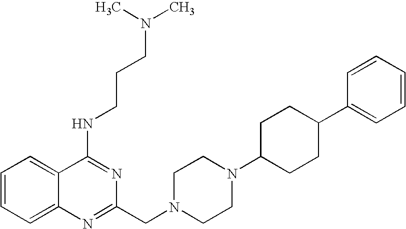 | 26-4 | 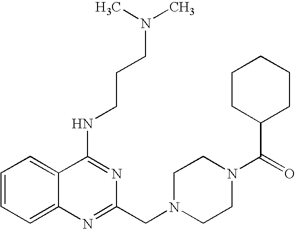 |
| 25-92 | 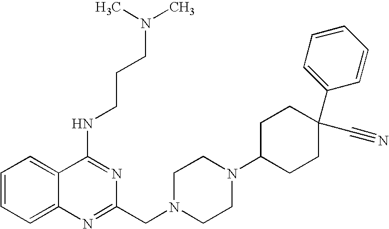 | 26-5 | 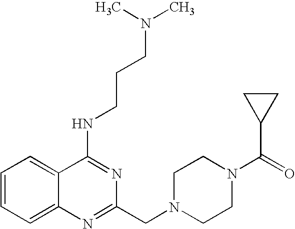 |
| 26-6 | 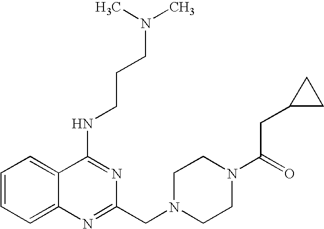 | 26-13 | 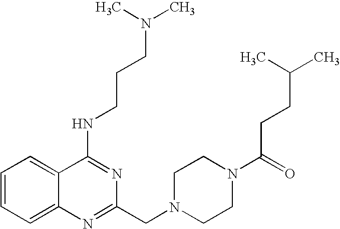 |
| 26-7 | 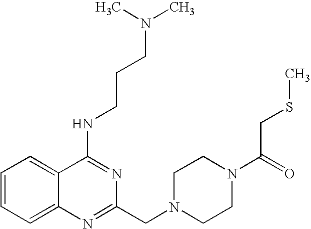 | 26-14 | 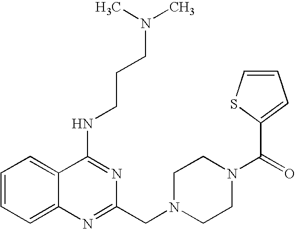 |
| 26-8 | 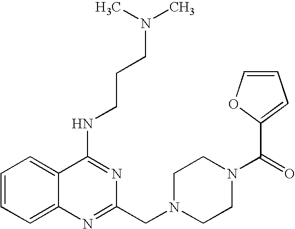 | 26-15 | 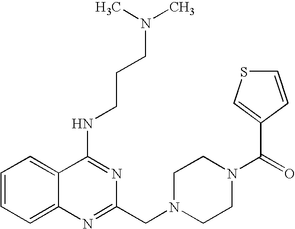 |
| 26-9 | 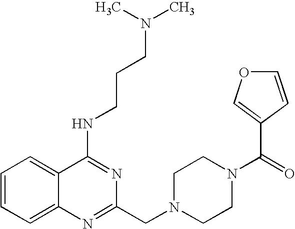 | 26-16 | 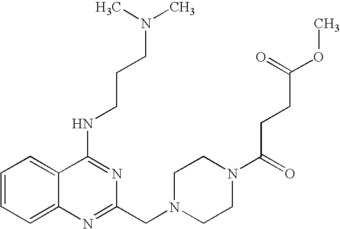 |
| 26-10 | 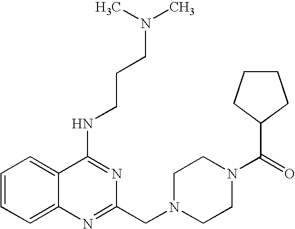 | 26-17 | 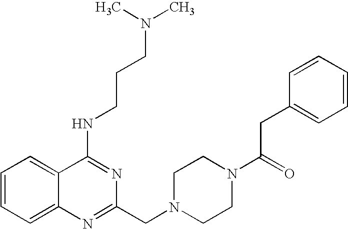 |
| 26-11 | 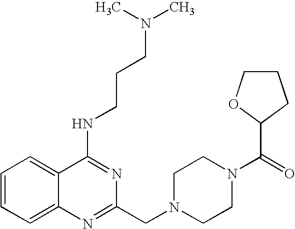 | 26-18 | 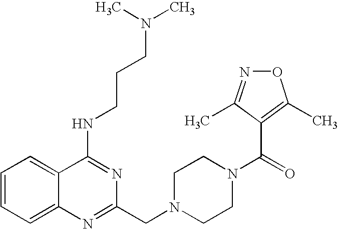 |
| 26-12 | 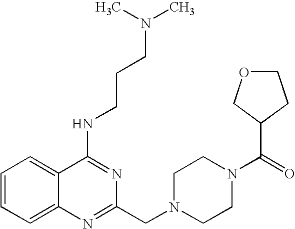 | 26-19 | 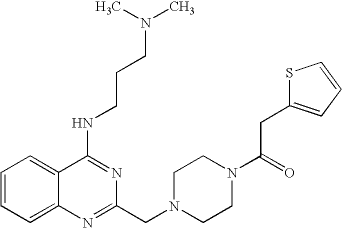 |
| 26-20 | 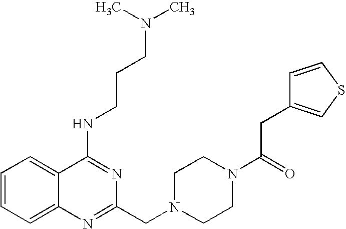 | 26-27 | 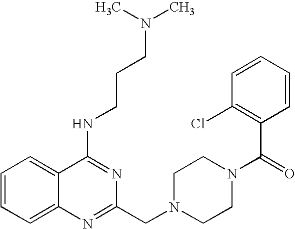 |
| 26-21 | 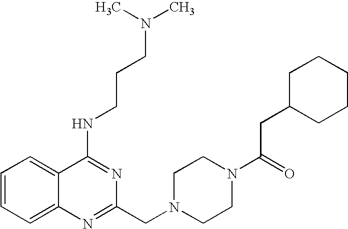 | 26-28 | 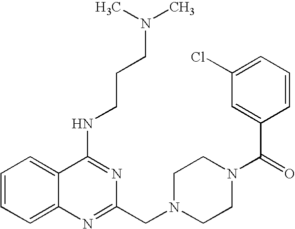 |
| 26-22 | 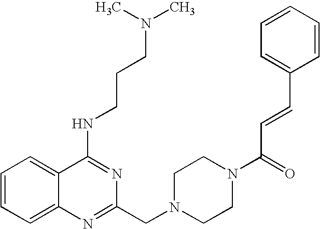 | 26-29 | 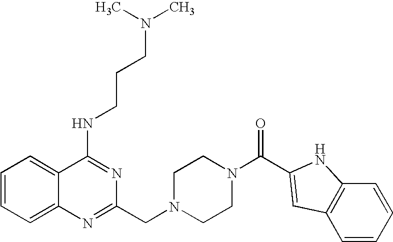 |
| 26-23 | 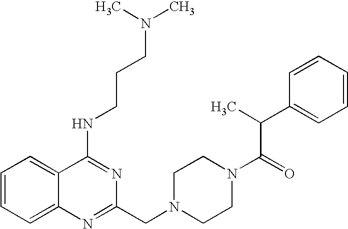 | 26-30 | 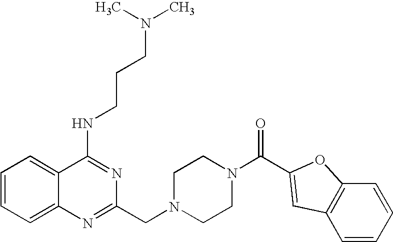 |
| 26-24 | 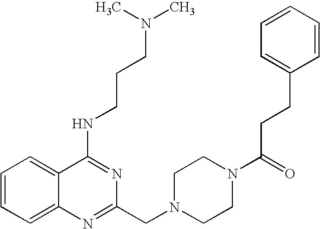 | 26-31 | 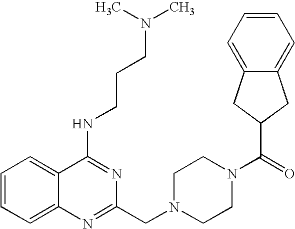 |
| 26-25 | 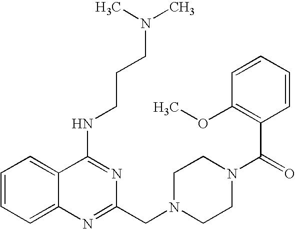 | 26-32 | 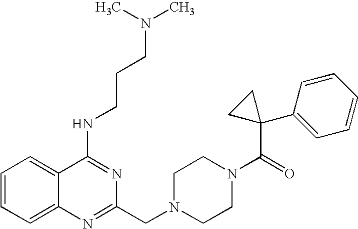 |
| 26-26 | 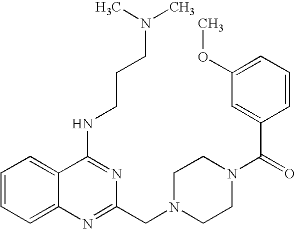 | 26-33 | 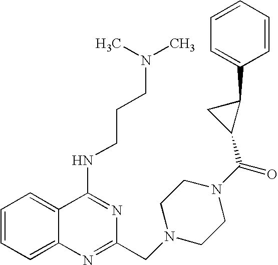 |
| 26-34 | 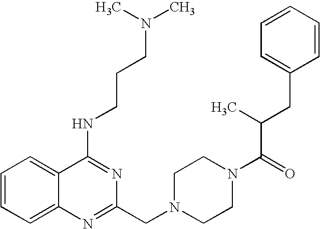 | 26-41 | 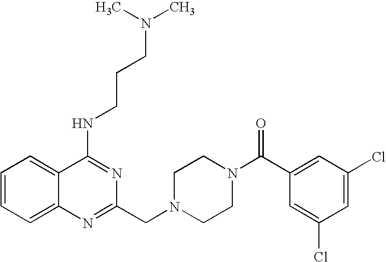 |
| 26-35 | 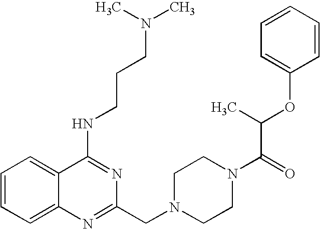 | 26-42 | 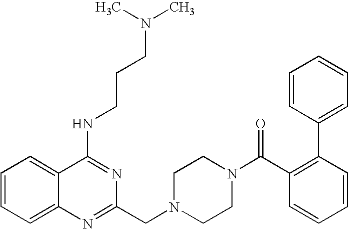 |
| 26-36 | 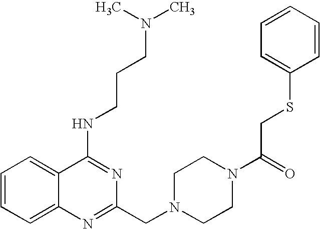 | 26-43 | 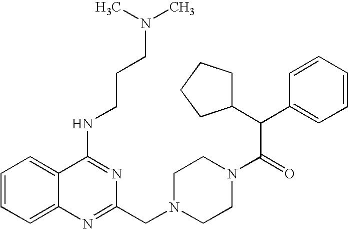 |
| 26-37 | 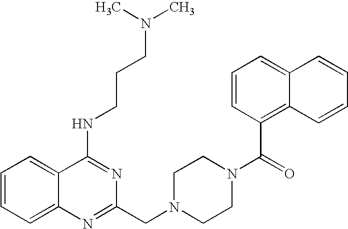 | 26-44 | 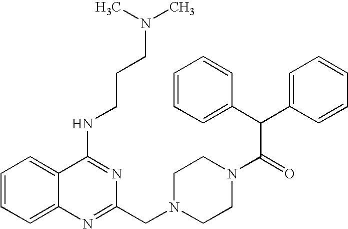 |
| 26-38 | 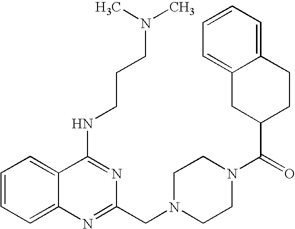 | 26-45 | 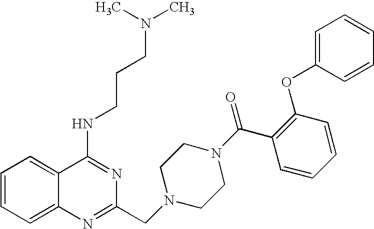 |
| 26-39 | 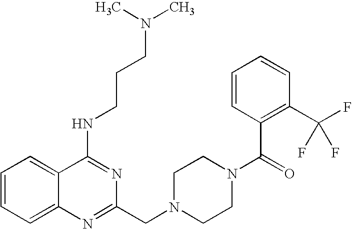 | 26-46 | 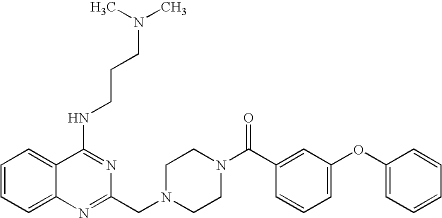 |
| 26-40 | 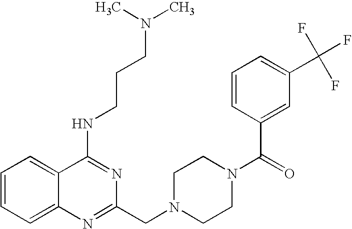 | 26-47 | 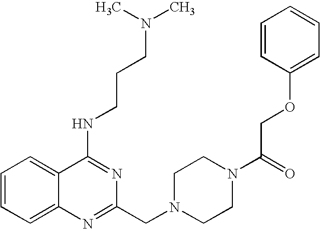 |
| 26-48 | 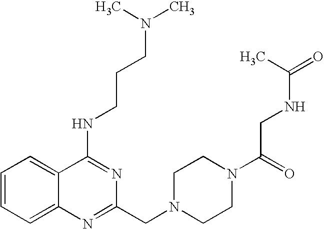 | 26-55 | 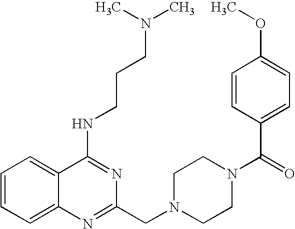 |
| 26-49 | 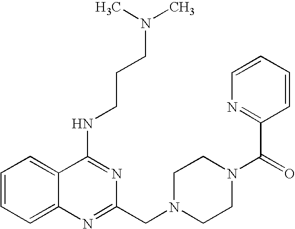 | 26-56 | 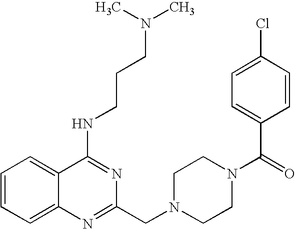 |
| 26-50 | 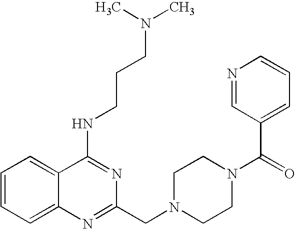 | 26-57 | 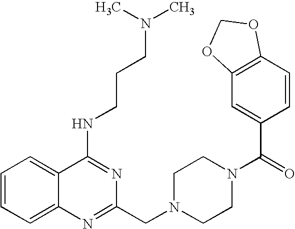 |
| 26-51 | 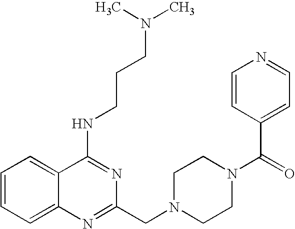 | 26-58 | 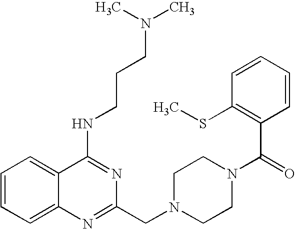 |
| 26-52 | 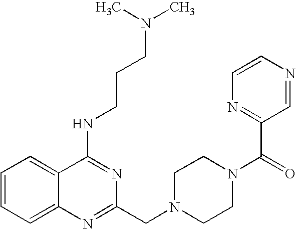 | 26-59 | 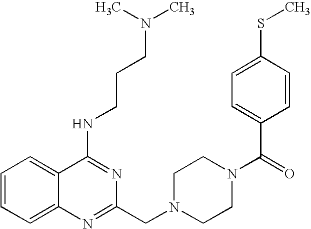 |
| 26-53 | 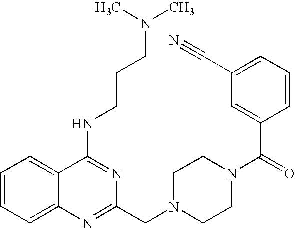 | 26-60 | 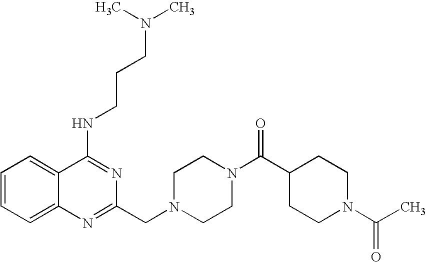 |
| 26-54 | 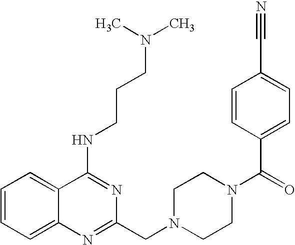 | 26-61 | 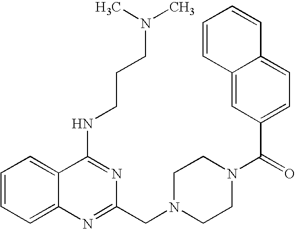 |
| 26-62 | 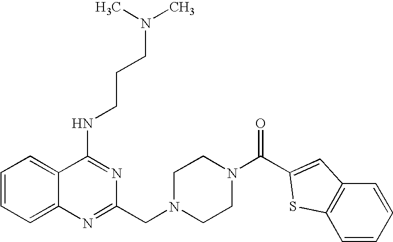 | 26-69 | 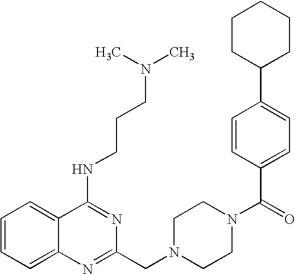 |
| 26-63 | 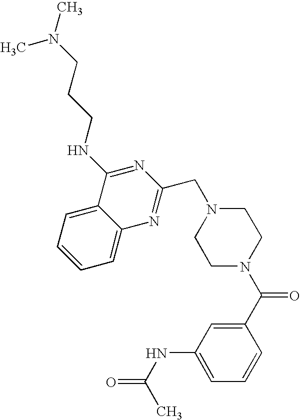 | 26-70 | 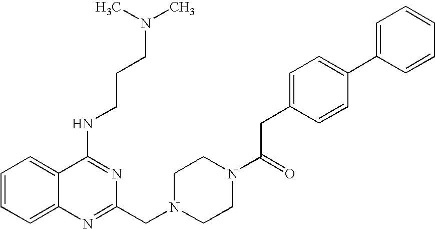 |
| 26-64 | 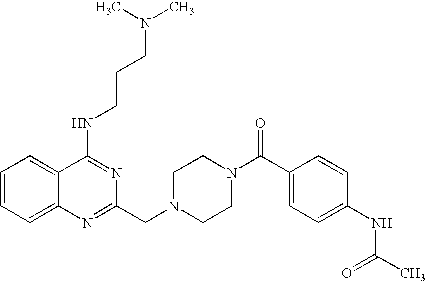 | 27-1 | 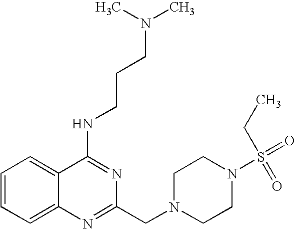 |
| 26-65 | 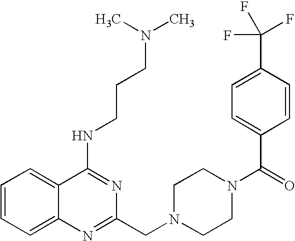 | 27-2 | 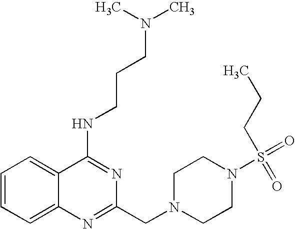 |
| 26-66 | 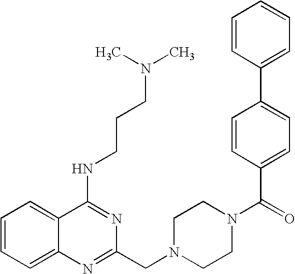 | 27-3 | 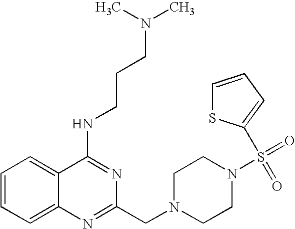 |
| 26-67 | 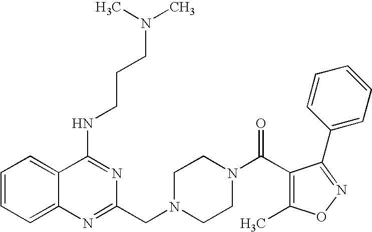 | 27-4 | 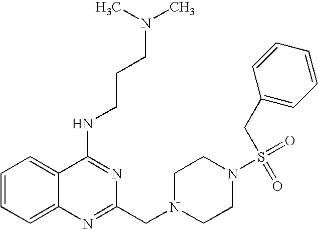 |
| 26-68 | 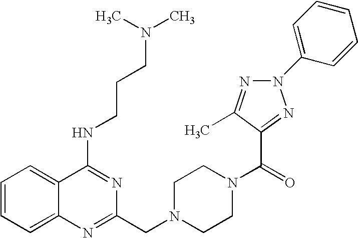 | 27-5 | 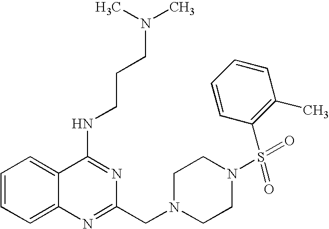 |
| 27-6 | 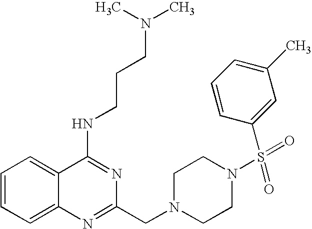 | 27-13 | 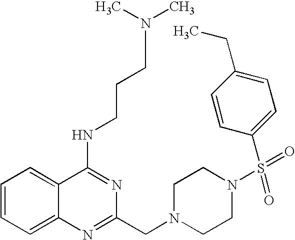 |
| 27-7 | 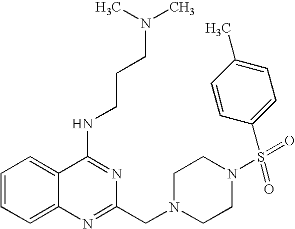 | 27-14 | 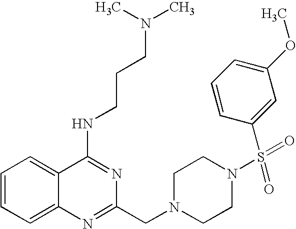 |
| 27-8 | 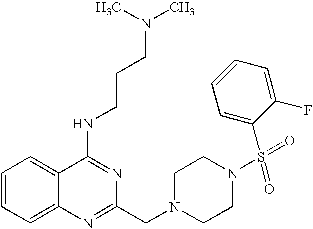 | 27-15 | 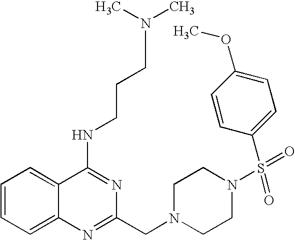 |
| 27-9 | 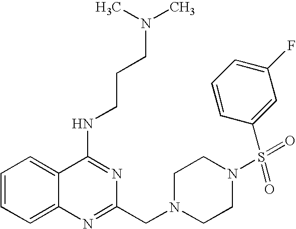 | 27-16 | 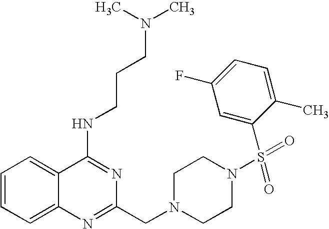 |
| 27-10 | 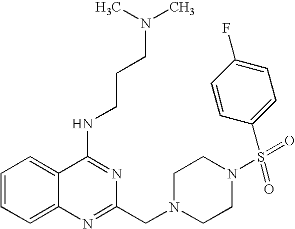 | 27-17 | 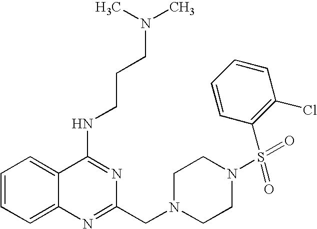 |
| 27-11 | 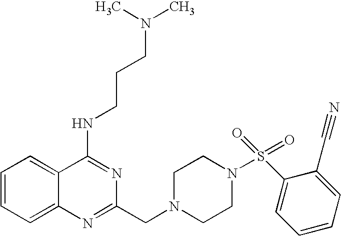 | 27-18 | 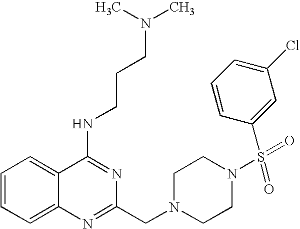 |
| 27-12 | 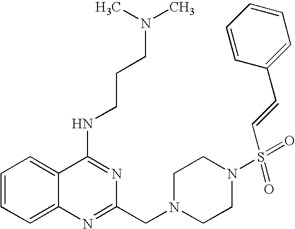 | 27-19 | 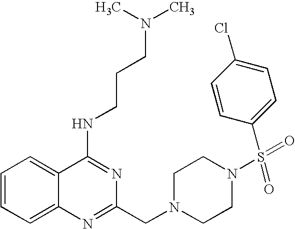 |
| 27-20 | 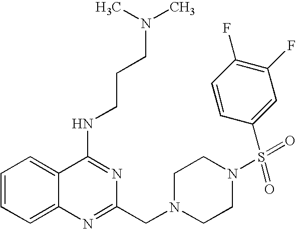 | 27-27 | 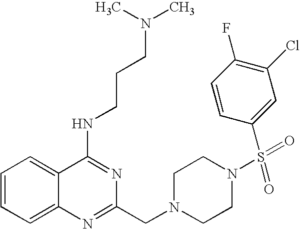 |
| 27-21 | 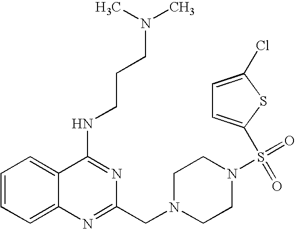 | 27-28 | 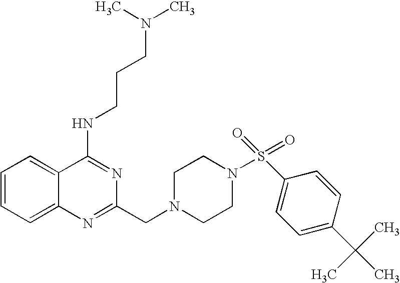 |
| 27-22 | 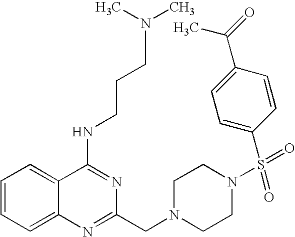 | 27-29 | 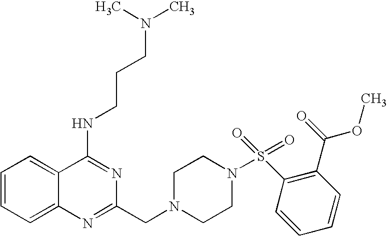 |
| 27-23 | 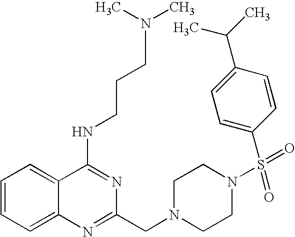 | 27-30 | 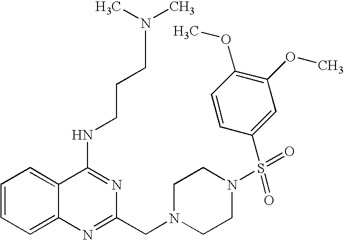 |
| 27-24 | 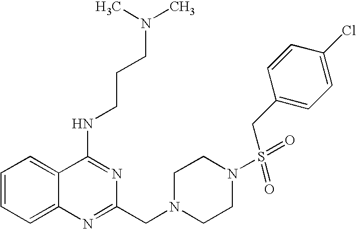 | 27-31 | 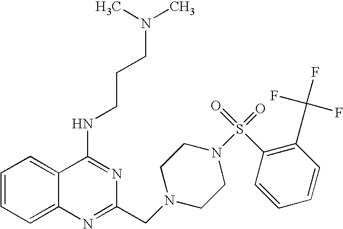 |
| 27-25 | 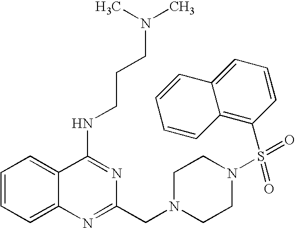 | 27-32 | 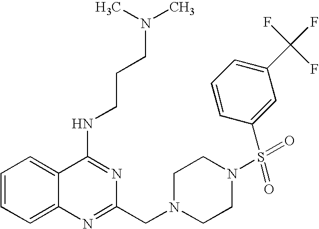 |
| 27-26 | 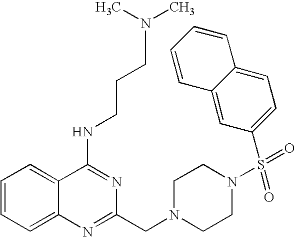 | 27-33 | 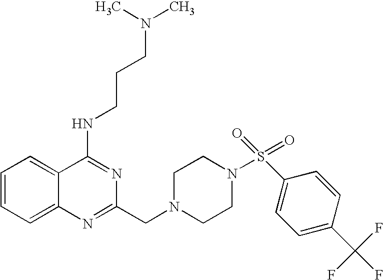 |
| 27-34 | 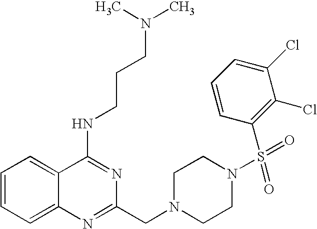 | 27-41 | 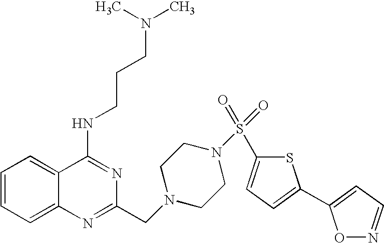 |
| 27-35 | 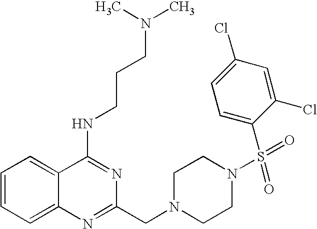 | 27-42 | 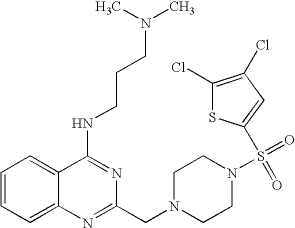 |
| 27-36 | 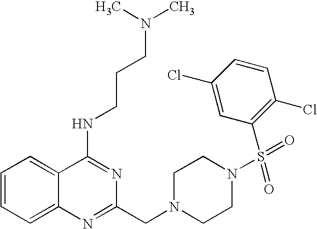 | 27-43 | 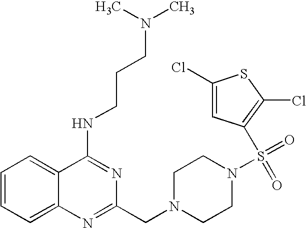 |
| 27-37 | 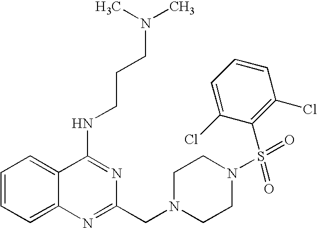 | 27-44 | 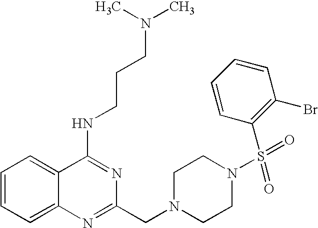 |
| 27-38 | 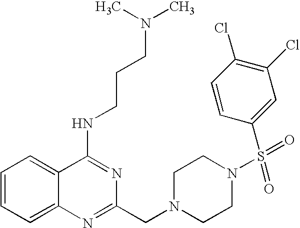 | 27-45 | 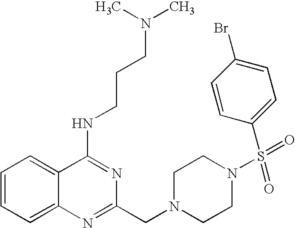 |
| 27-39 | 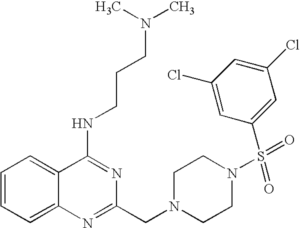 | 27-46 | 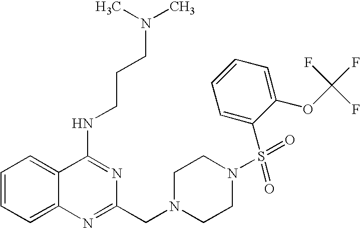 |
| 27-40 | 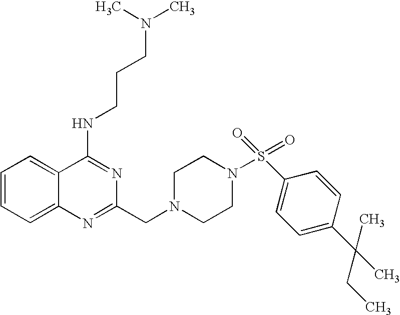 | 27-47 | 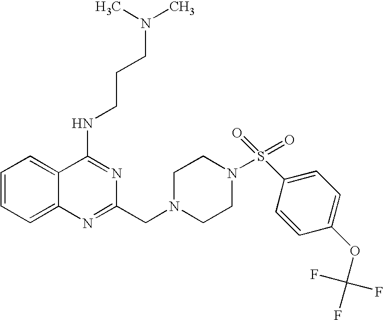 |
| 27-48 | 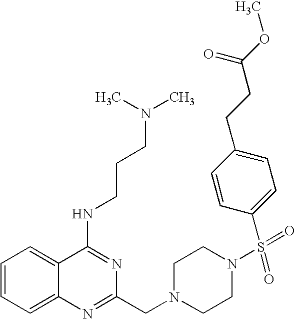 | 27-55 | 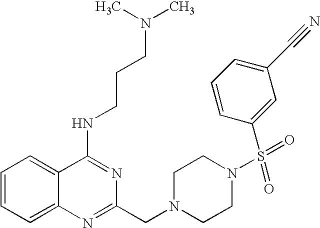 |
| 27-49 | 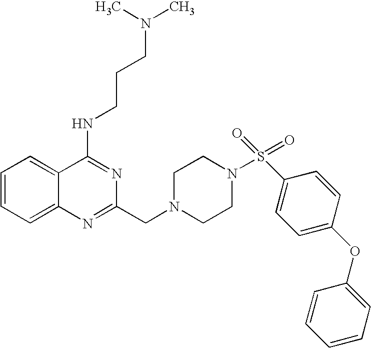 | 27-56 | 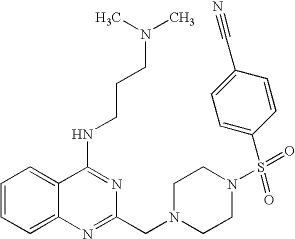 |
| 27-50 | 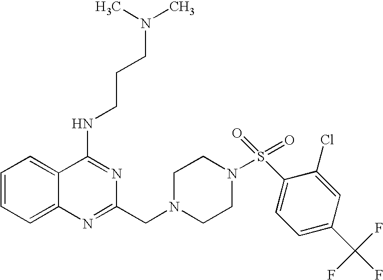 | 27-57 | 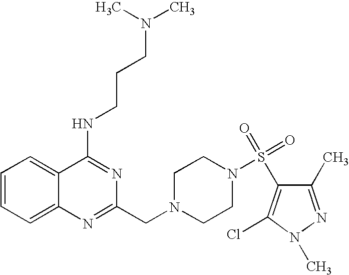 |
| 27-51 | 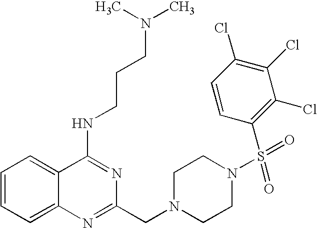 | 27-58 | 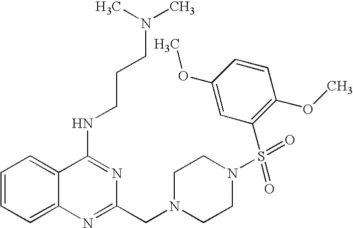 |
| 27-52 | 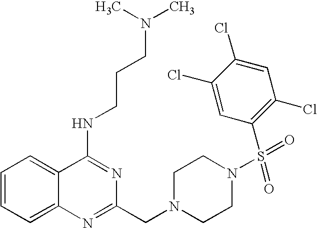 | 27-59 | 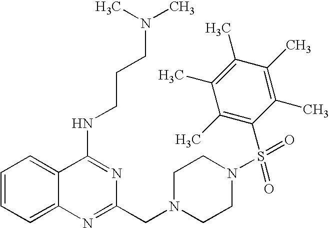 |
| 27-53 | 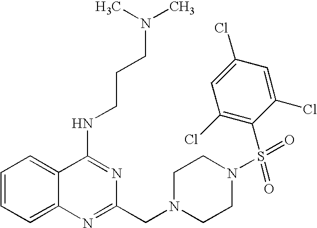 | 27-60 | 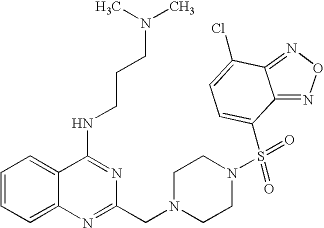 |
| 27-54 | 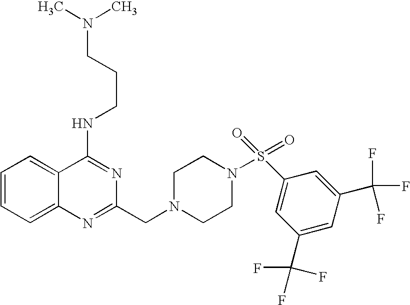 | 27-61 | 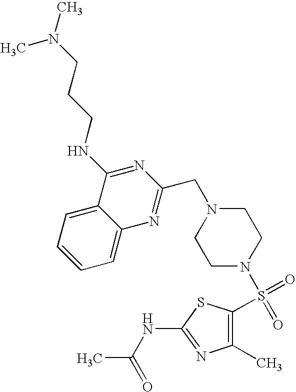 |
| 27-62 | 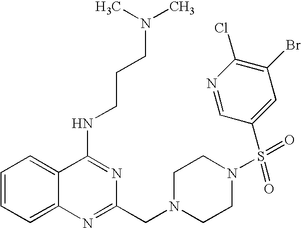 | 27-69 | 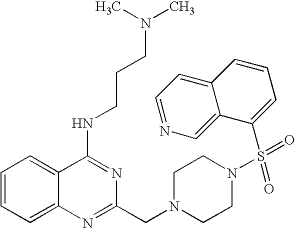 |
| 27-63 | 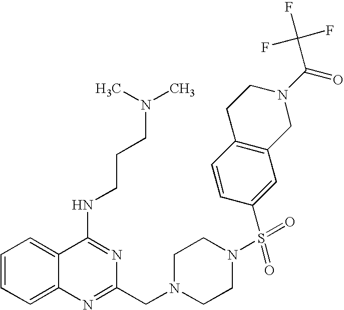 | 27-70 | 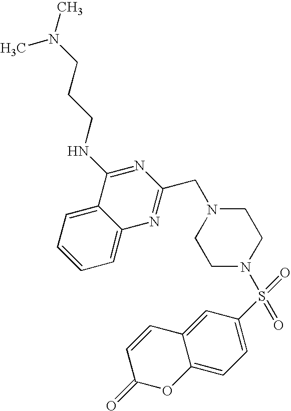 |
| 27-64 | 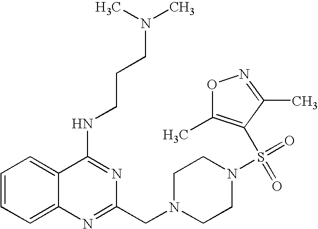 | 27-71 | 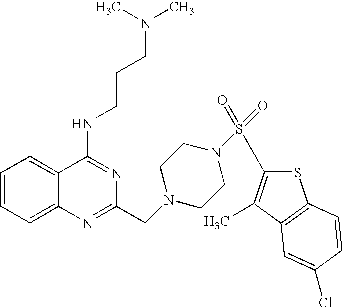 |
| 27-65 | 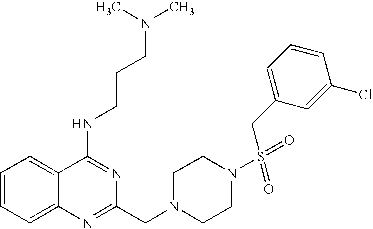 | 27-72 | 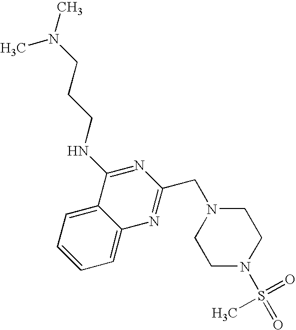 |
| 27-66 | 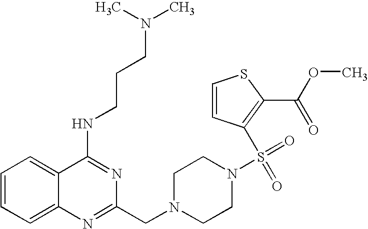 | 27-73 | 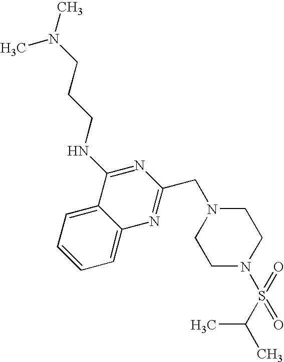 |
| 27-67 | 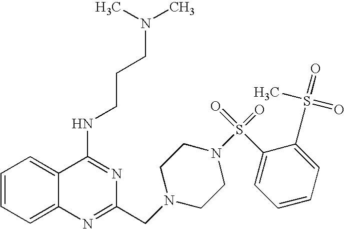 | 27-74 | 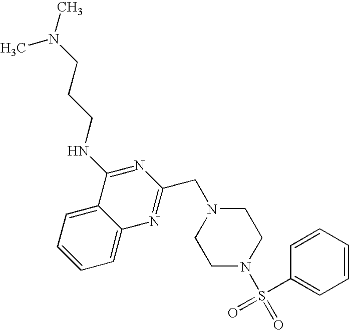 |
| 27-68 | 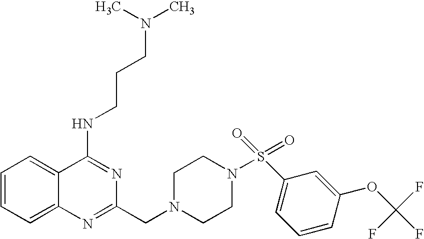 | 27-75 | 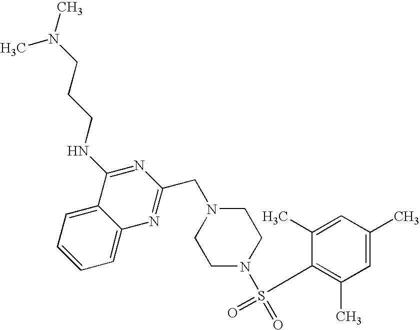 |
| 28-1 | 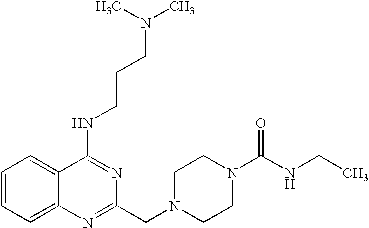 | 28-8 | 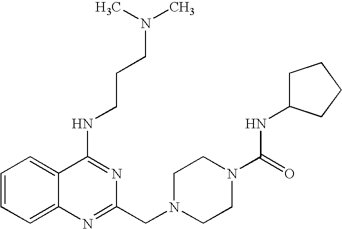 |
| 28-2 | 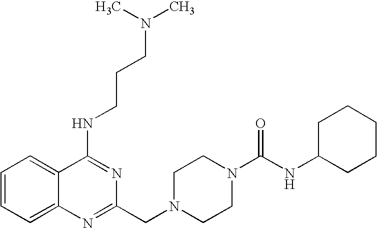 | 28-9 | 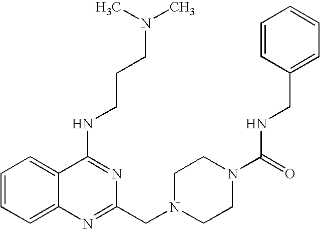 |
| 28-3 | 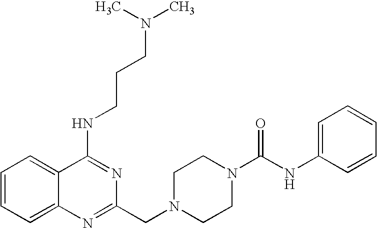 | 28-10 | 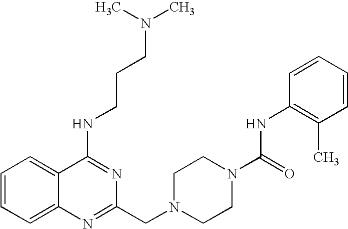 |
| 28-4 | 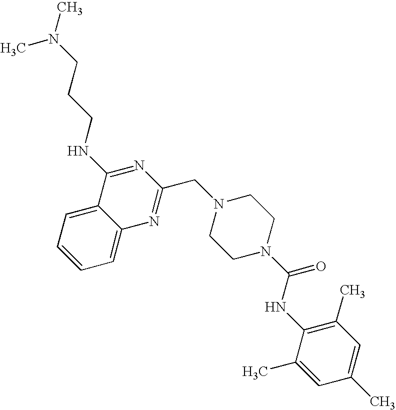 | 28-11 | 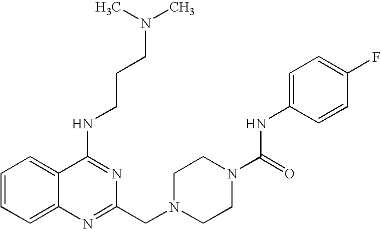 |
| 28-5 | 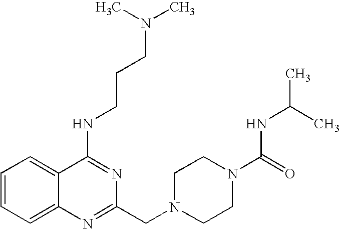 | 28-12 | 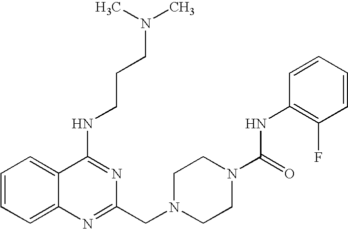 |
| 28-6 | 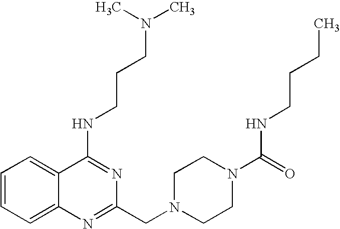 | 28-13 | 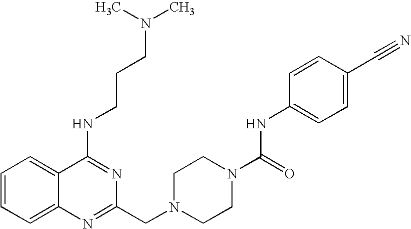 |
| 28-7 | 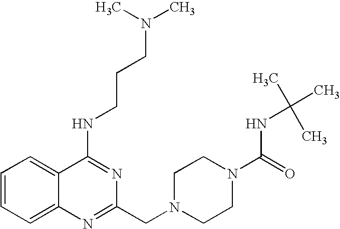 | 28-14 | 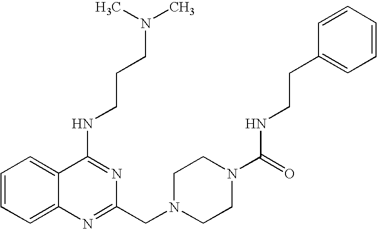 |
| 28-15 | 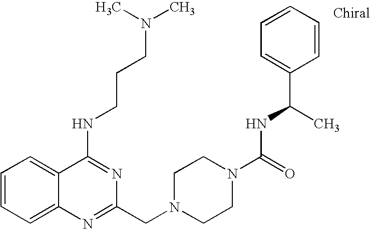 | 28-22 | 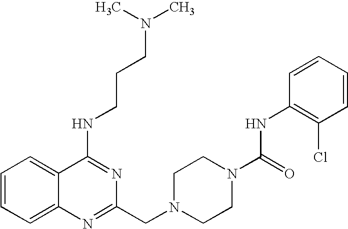 |
| 28-16 | 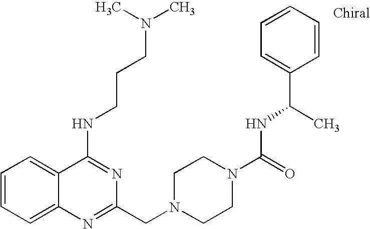 | 28-23 | 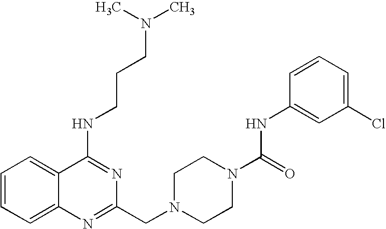 |
| 28-31 | 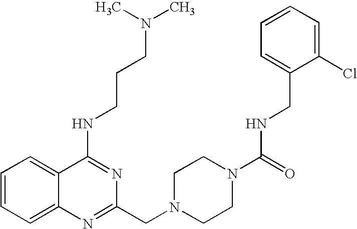 | 28-38 | 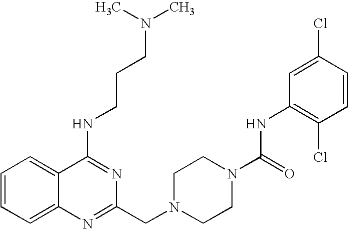 |
| 28-32 | 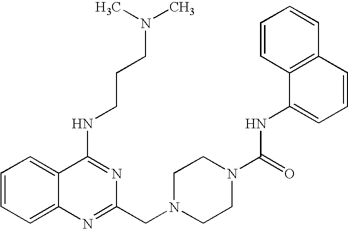 | 28-39 | 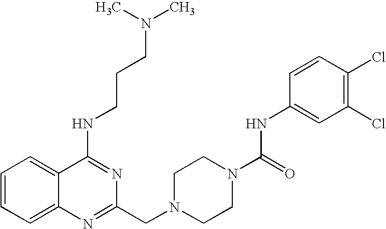 |
| 28-33 | 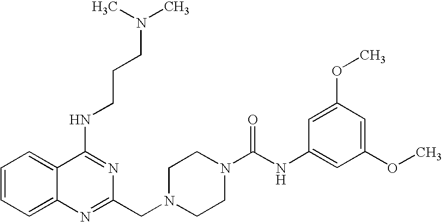 | 28-40 | 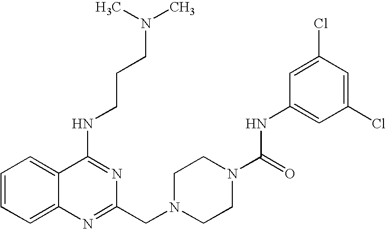 |
| 28-34 | 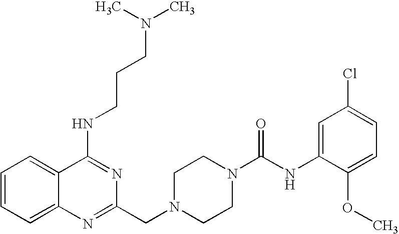 | 28-41 | 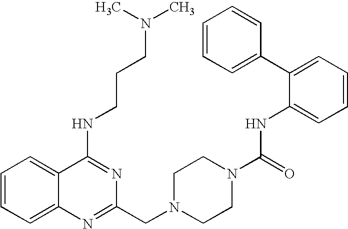 |
| 28-35 | 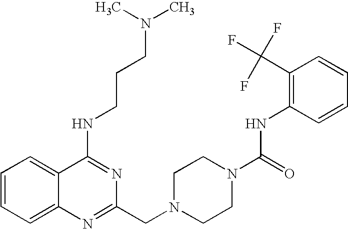 | 28-42 | 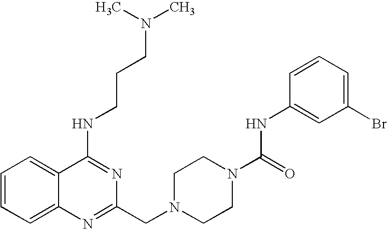 |
| 28-43 | 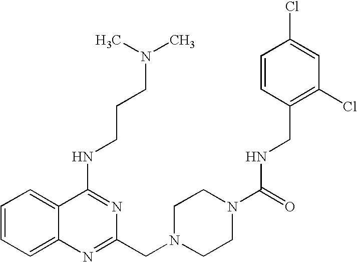 | 29-2 | 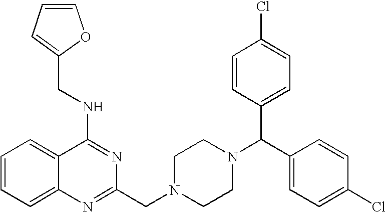 |
| 28-44 | 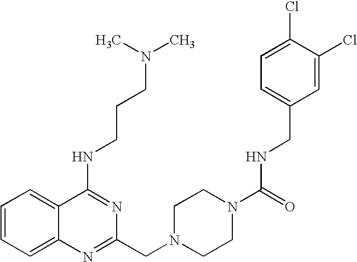 | 29-3 | 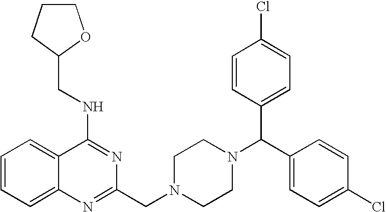 |
| 28-45 | 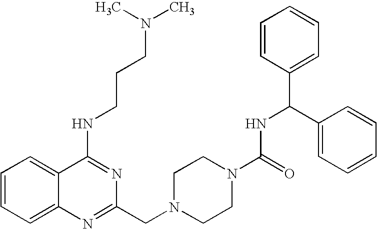 | 29-4 | 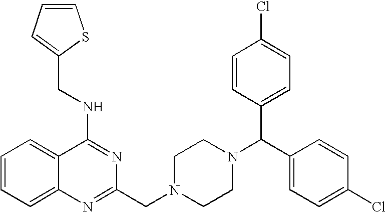 |
| 28-46 | 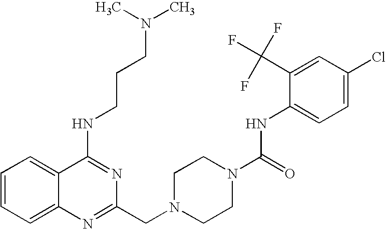 | 29-5 | 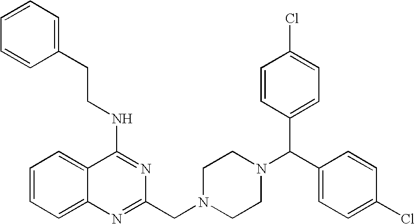 |
| 28-47 | 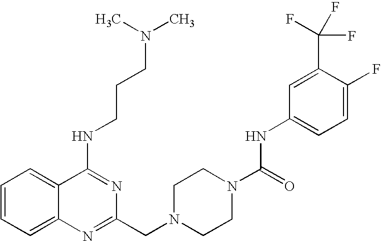 | 29-6 | 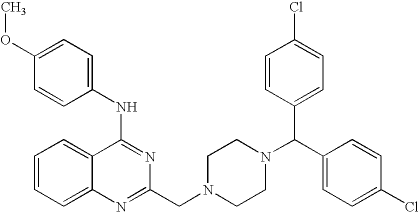 |
| 28-48 | 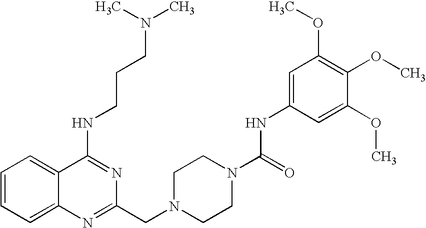 | 29-7 | 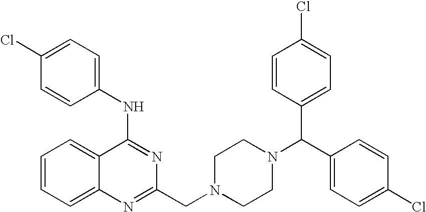 |
| 29-1 | 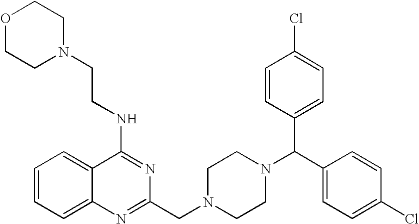 | 29-8 | 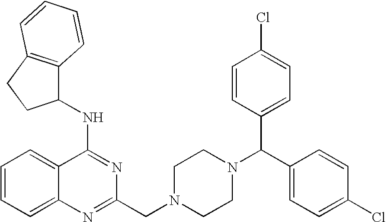 |
| 29-9 | 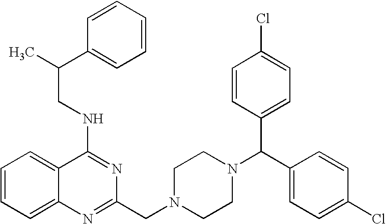 | 29-16 | 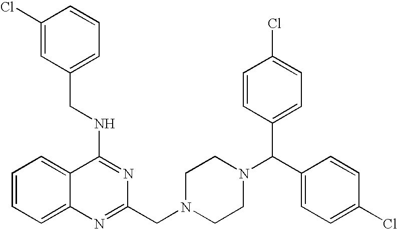 |
| 29-10 | 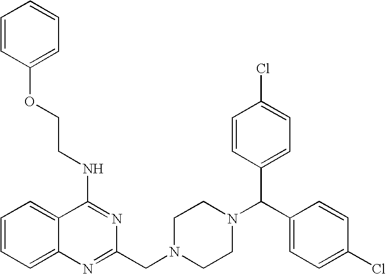 | 29-17 | 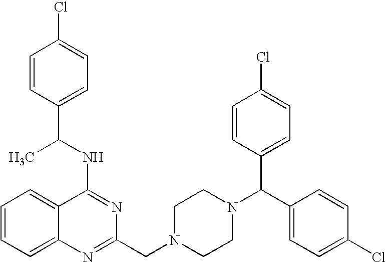 |
| 29-11 | 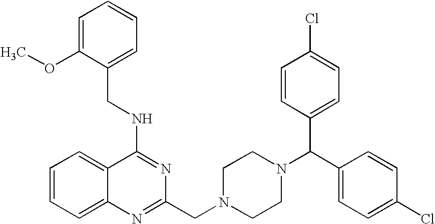 | 29-18 | 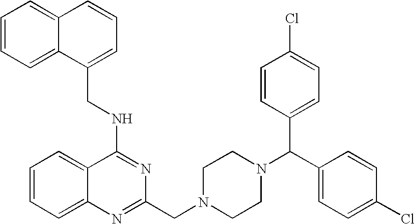 |
| 29-12 | 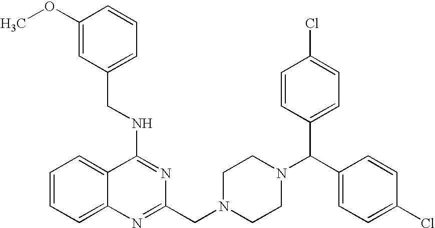 | 29-19 | 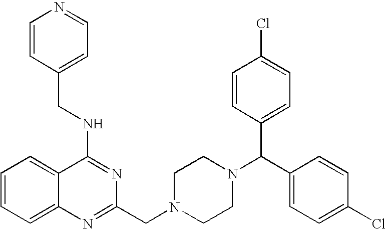 |
| 29-13 | 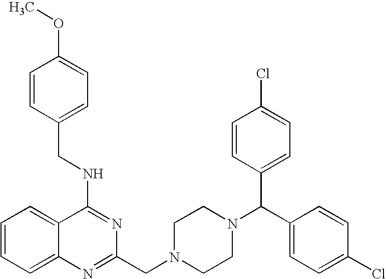 | 29-20 | 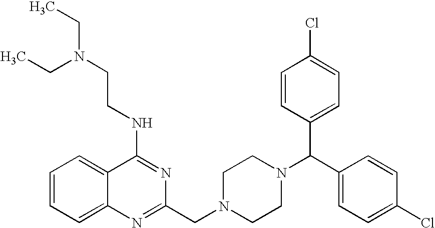 |
| 29-14 | 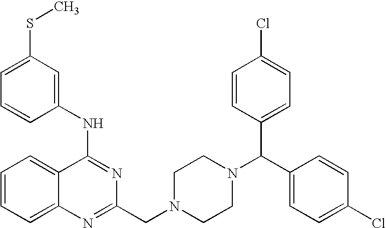 | 29-21 | 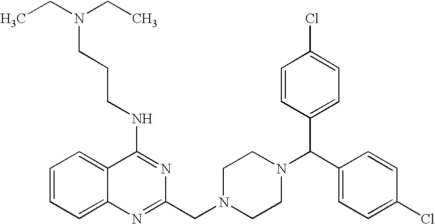 |
| 29-15 | 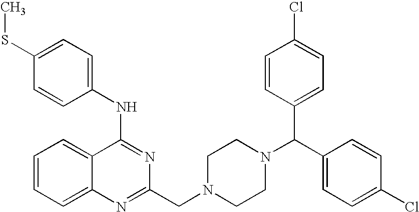 | 29-22 | 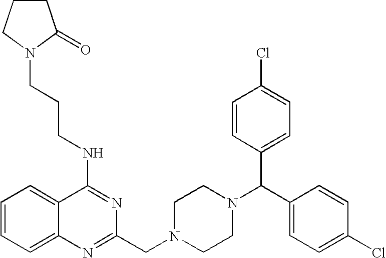 |
| 29-23 | 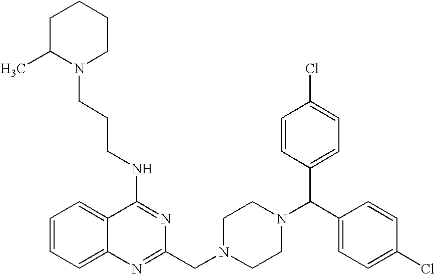 | 29-30 | 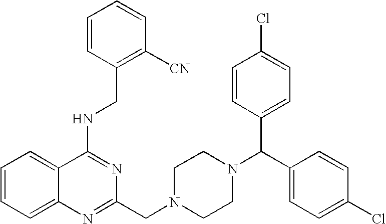 |
| 29-24 | 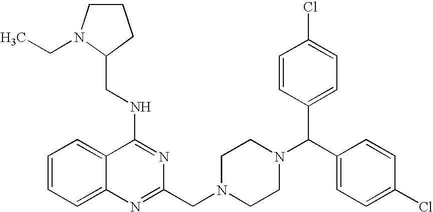 | 29-31 | 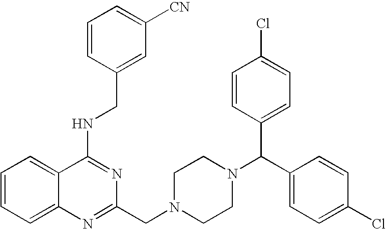 |
| 29-25 | 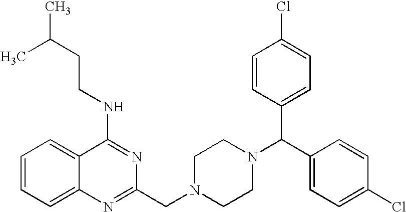 | 29-32 | 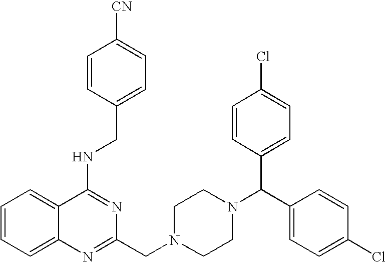 |
| 29-26 | 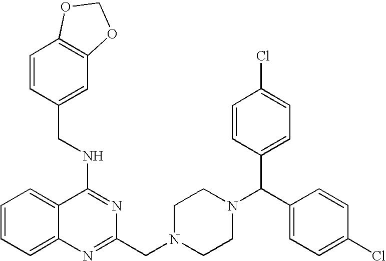 | 29-33 | 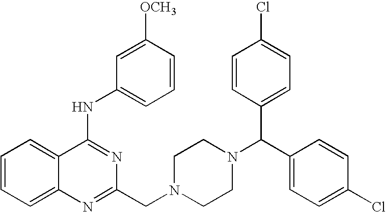 |
| 29-27 | 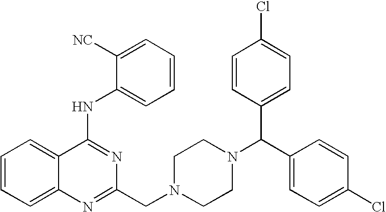 | 29-34 | 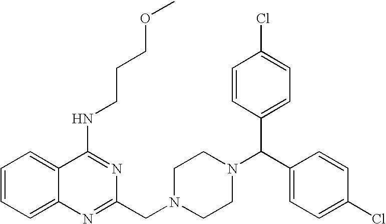 |
| 29-28 | 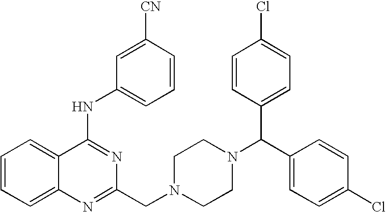 | 29-35 | 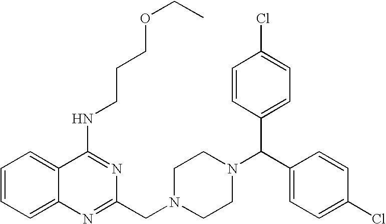 |
| 29-29 | 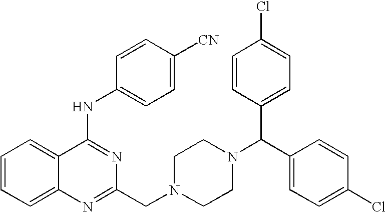 | 29-36 | 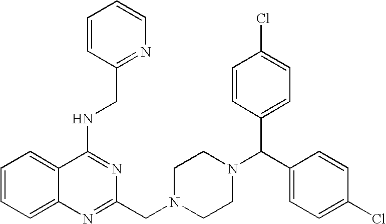 |
| 29-37 | 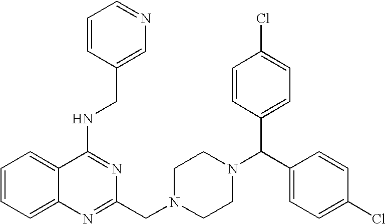 | 29-44 | 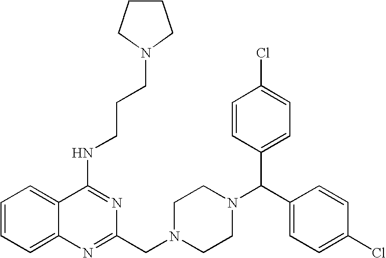 |
| 29-38 | 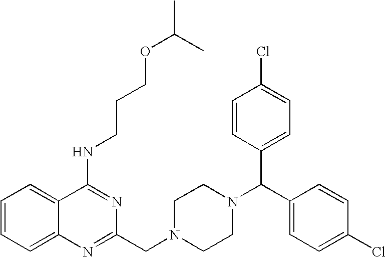 | 29-45 | 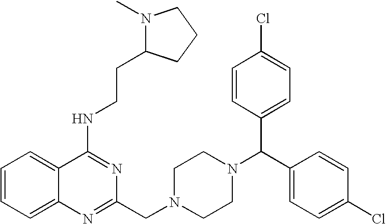 |
| 29-39 | 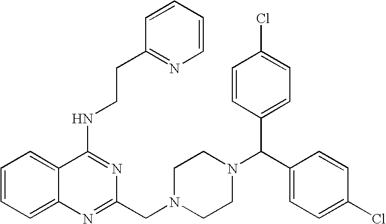 | 29-46 | 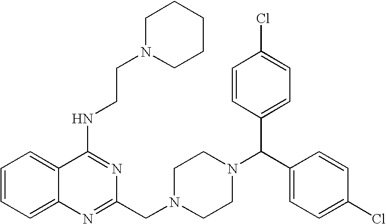 |
| 29-40 | 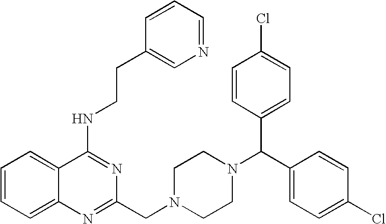 | 29-47 | 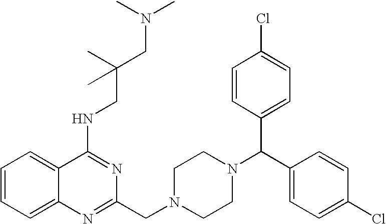 |
| 29-41 | 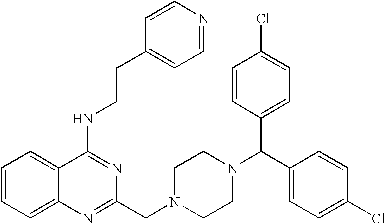 | 29-48 | 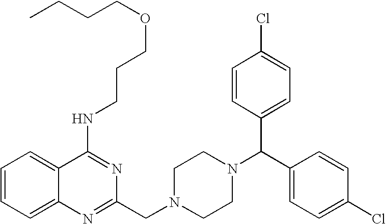 |
| 29-42 | 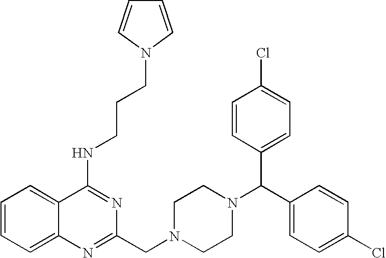 | 29-49 | 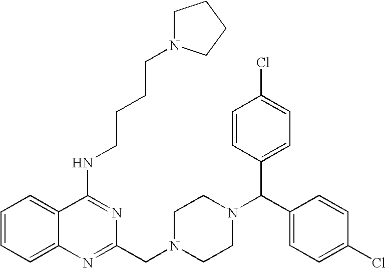 |
| 29-43 | 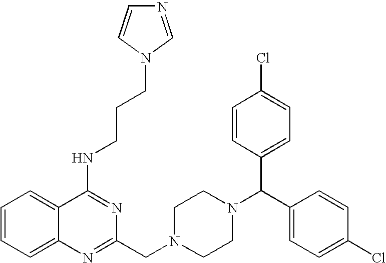 | 29-50 | 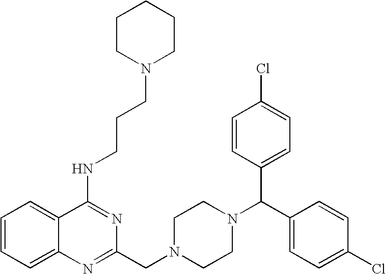 |
| 29-51 | 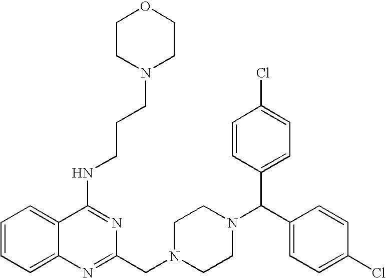 | 29-58 | 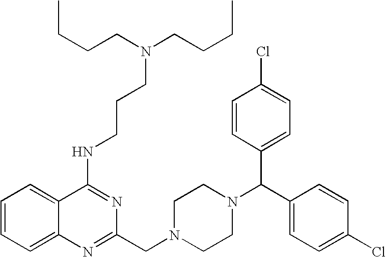 |
| 29-52 | 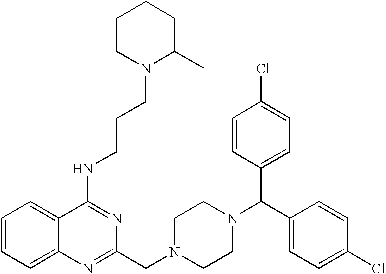 | 29-59 | 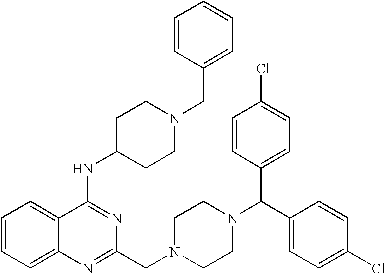 |
| 29-53 | 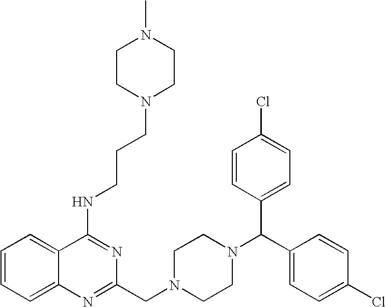 | 29-60 | 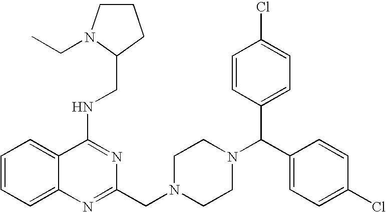 |
| 29-54 | 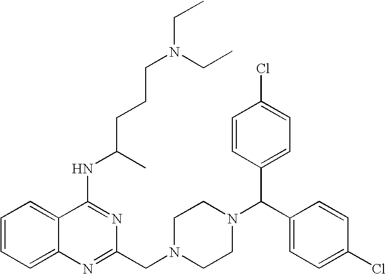 | 29-61 | 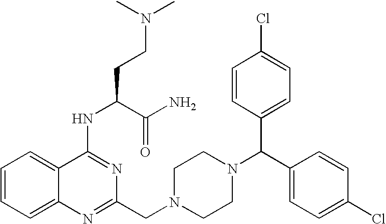 |
| 29-55 | 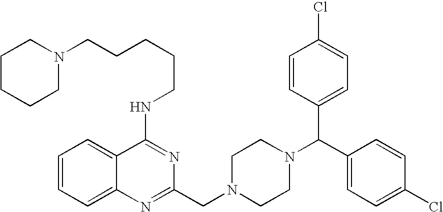 | 29-62 | 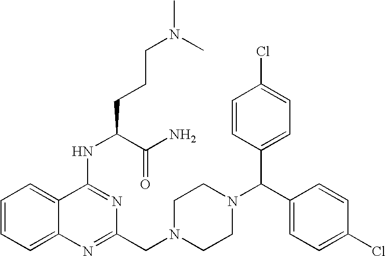 |
| 29-56 | 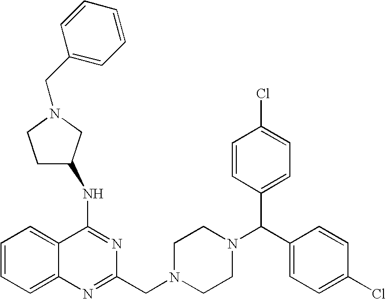 | 29-63 | 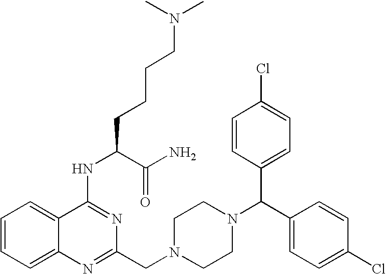 |
| 29-57 | 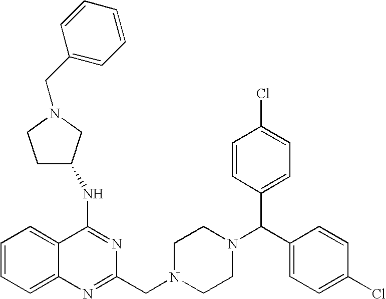 | 29-64 | 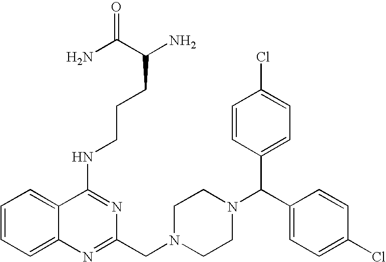 |
| 29-65 | 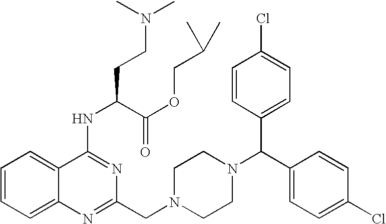 | 30-5 | 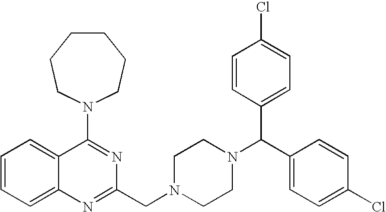 |
| 29-66 | 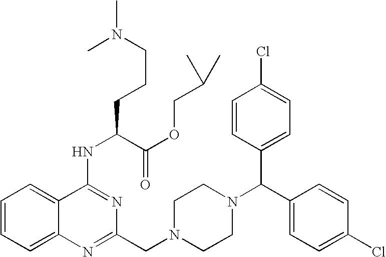 | 30-6 | 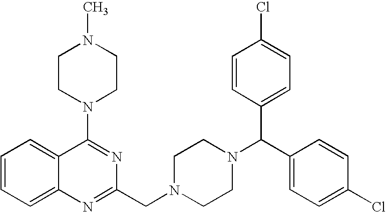 |
| 29-67 | 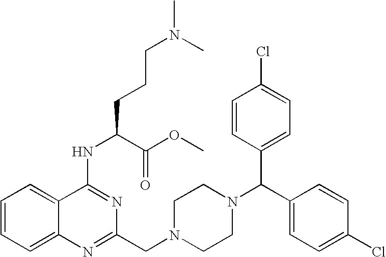 | 30-7 | 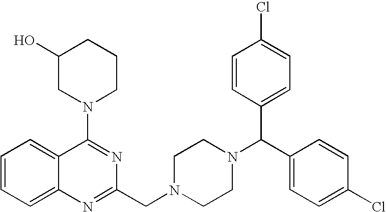 |
| 30-1 | 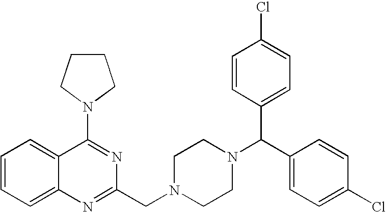 | 30-8 | 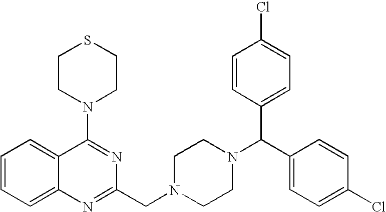 |
| 30-2 | 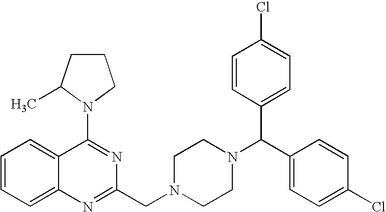 | 30-9 | 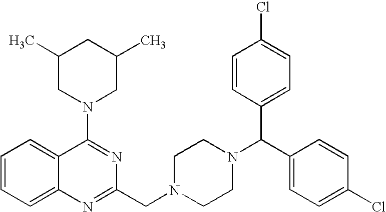 |
| 30-3 | 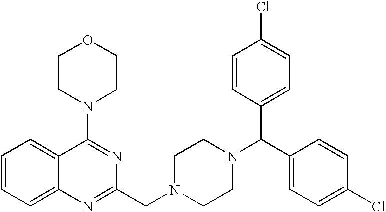 | 30-10 | 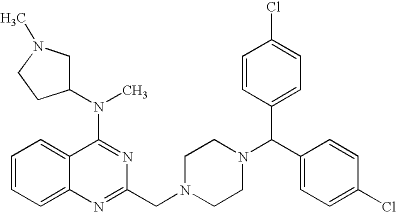 |
| 30-4 | 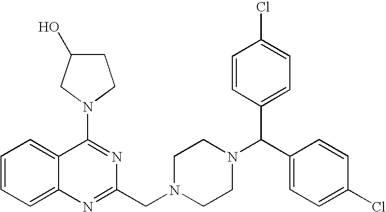 | 30-11 | 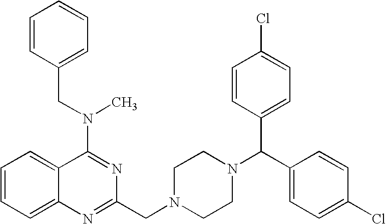 |
| 30-12 | 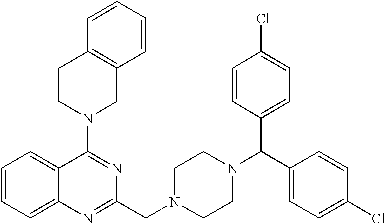 | 30-19 | 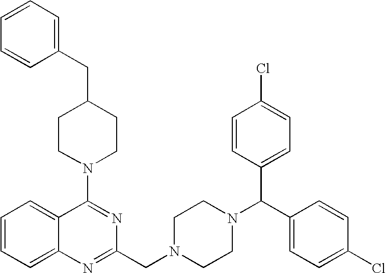 |
| 30-13 | 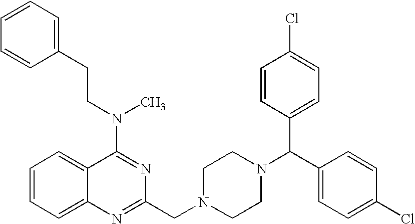 | 30-20 | 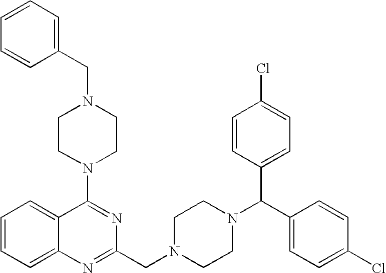 |
| 30-14 | 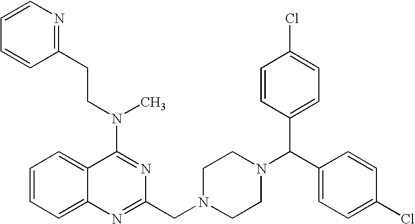 | 30-21 | 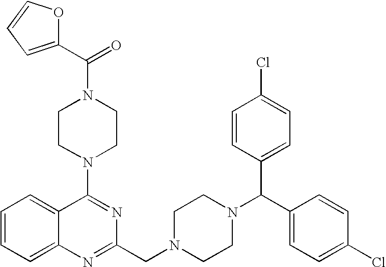 |
| 30-15 | 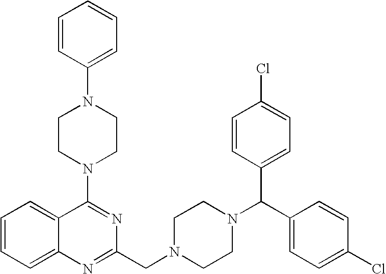 | 30-22 | 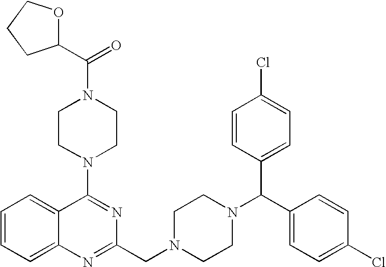 |
| 30-16 | 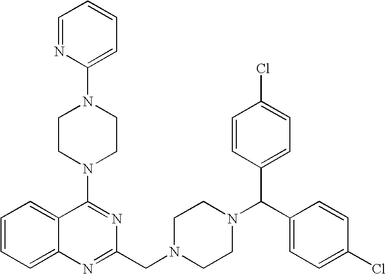 | 30-23 | 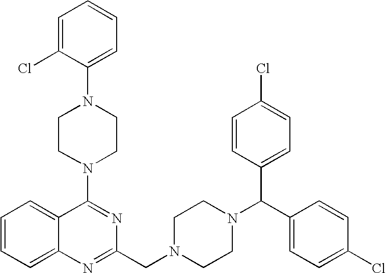 |
| 30-17 | 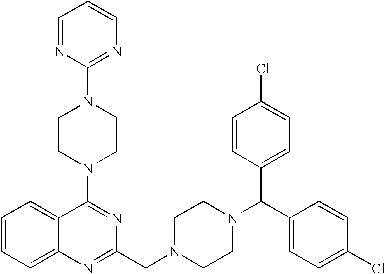 | 30-24 | 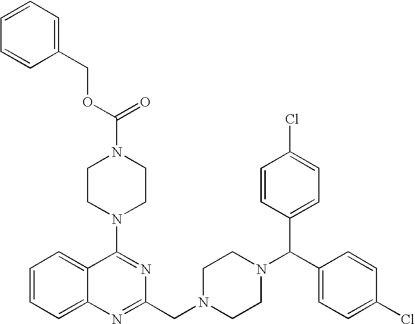 |
| 30-18 | 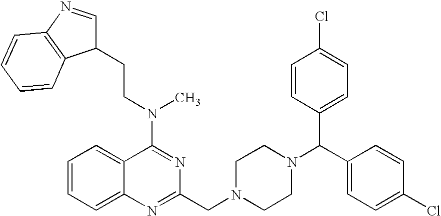 | 30-25 | 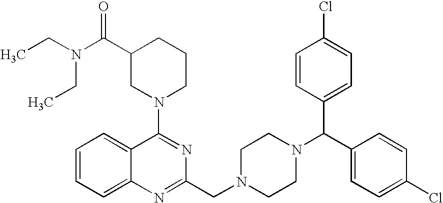 |
| 30-26 | 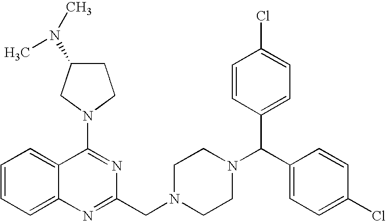 | 30-33 | 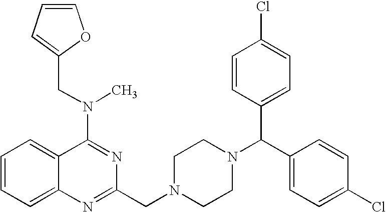 |
| 30-27 | 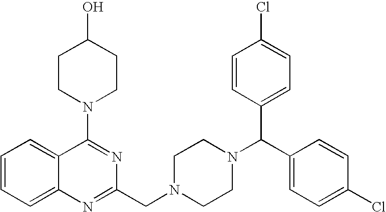 | 30-34 | 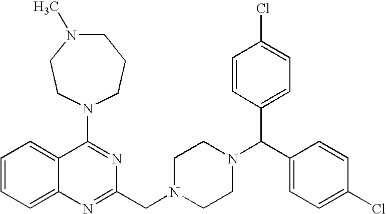 |
| 30-28 | 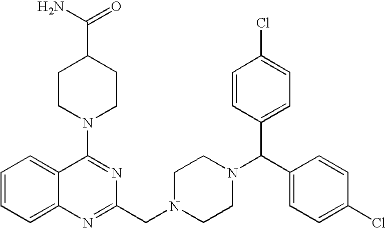 | 30-35 | 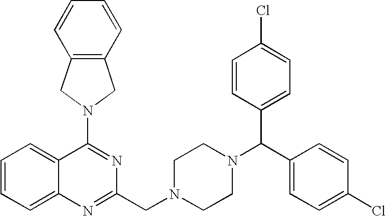 |
| 30-29 | 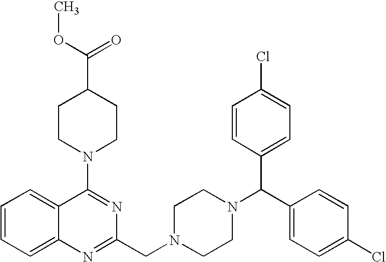 | 30-36 | 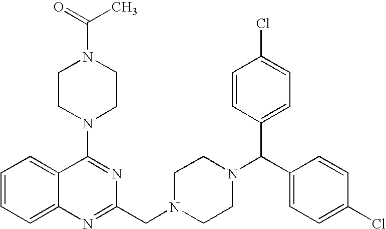 |
| 30-30 | 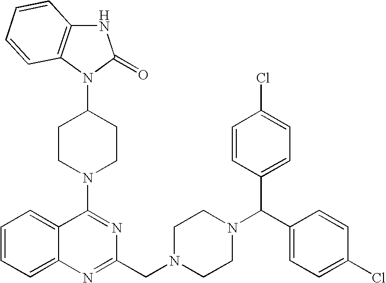 | 30-37 | 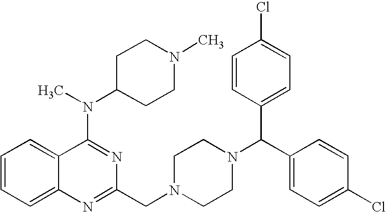 |
| 30-31 | 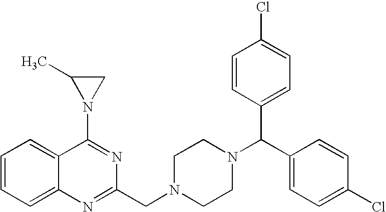 | 30-38 | 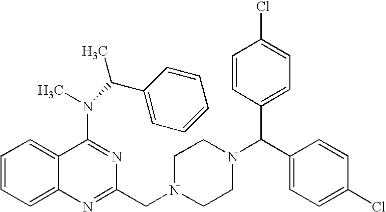 |
| 30-32 | 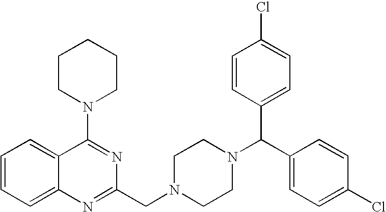 | 30-39 | 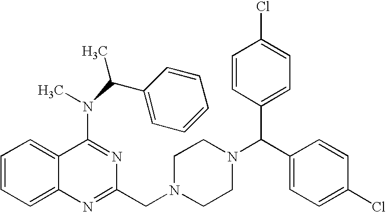 |
| 30-40 | 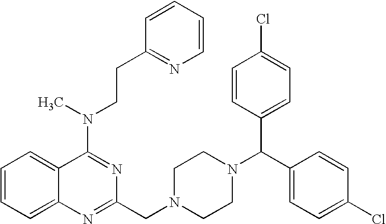 | 30-47 | 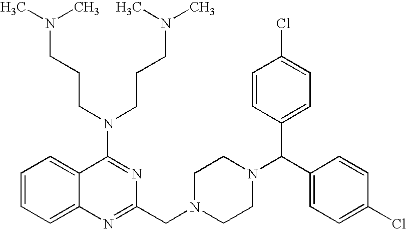 |
| 30-41 | 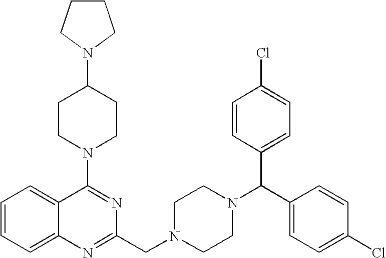 | 30-48 | 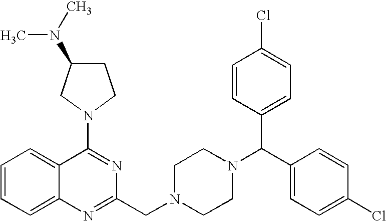 |
| 30-42 | 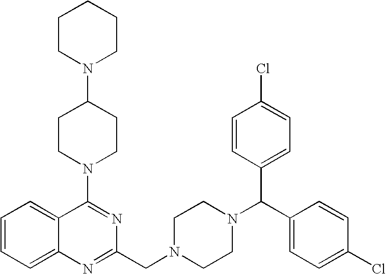 | 30-49 | 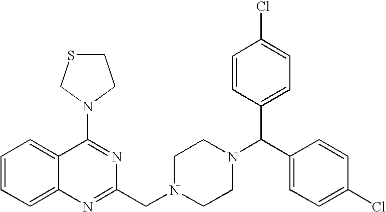 |
| 30-43 | 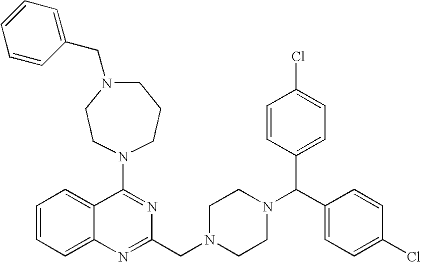 | 30-50 | 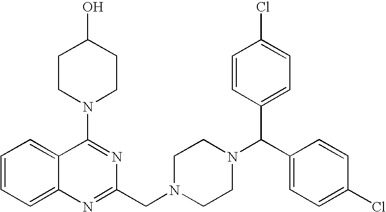 |
| 30-44 | 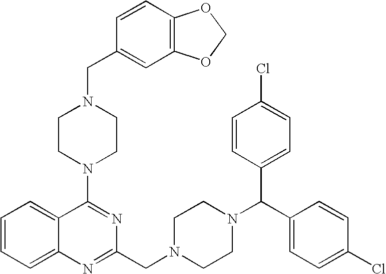 | 30-51 | 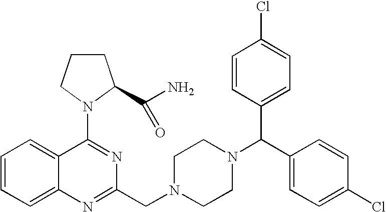 |
| 30-45 | 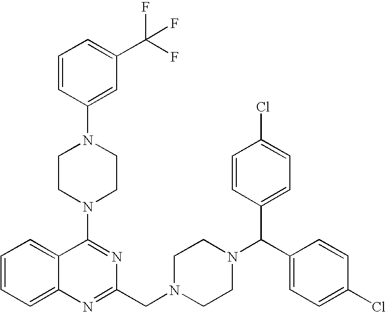 | 30-52 | 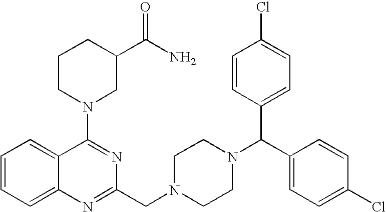 |
| 30-46 | 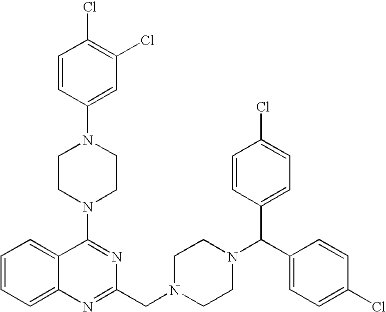 | 30-53 | 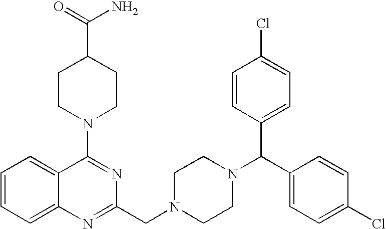 |
| 30-54 | 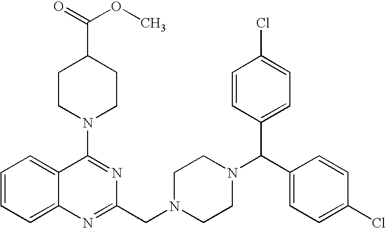 | 44 | 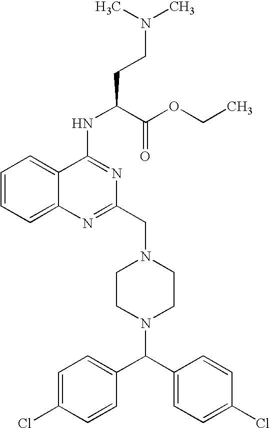 |
| 30-55 | 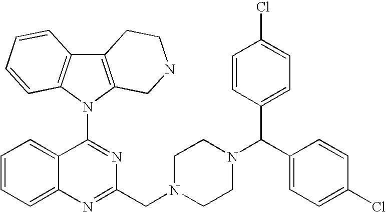 | 45 | 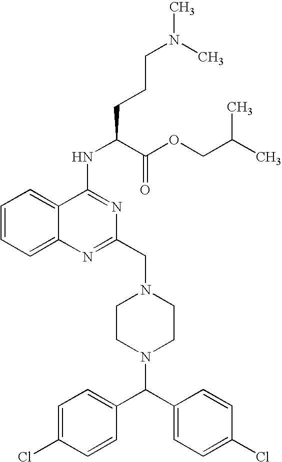 |
| 30-56 | 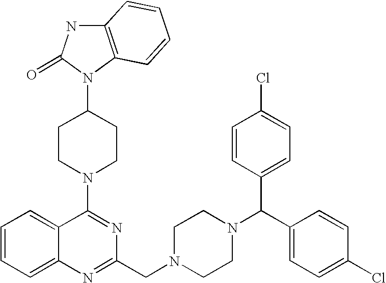 | 46 | 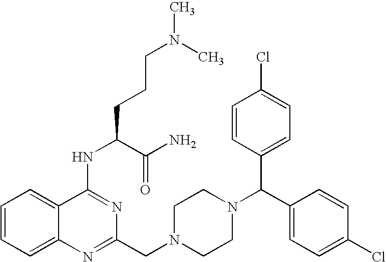 |
| 30-57 | 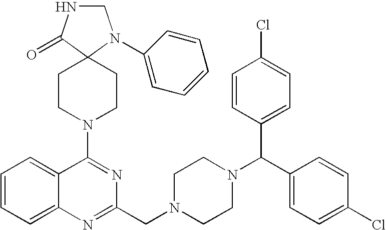 | 47 | 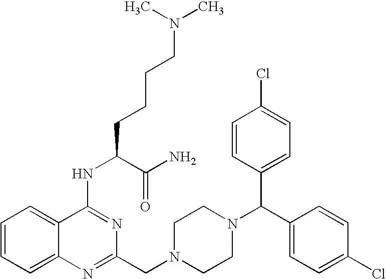 |
| 30-58 | 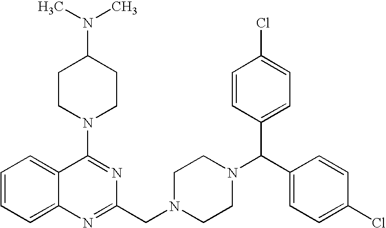 | | |
| 43 | 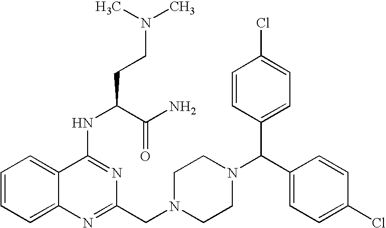 | | |

or a pharmaceutically acceptable salt, solvate or ester thereof.

41. The compound of claim 40, wherein said compound is selected from the group consisting of

| COMPOUND # | STRUCTURE |
|---|---|
| 1 | 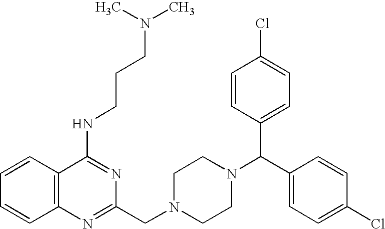 |
| 11-1 | 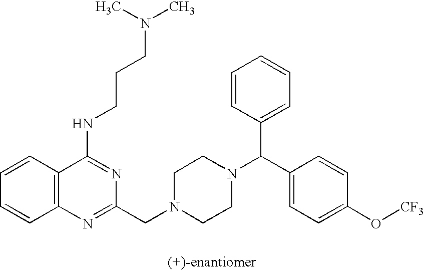 |
| 11-2 | 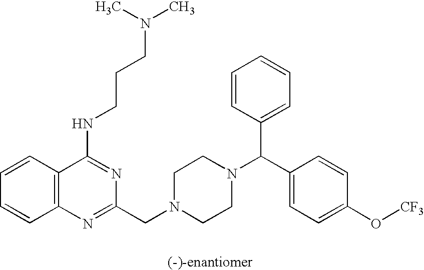 |
| 20-29 | 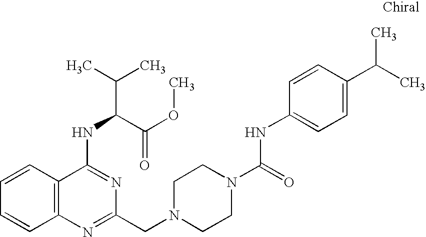 |
| 20-46 | 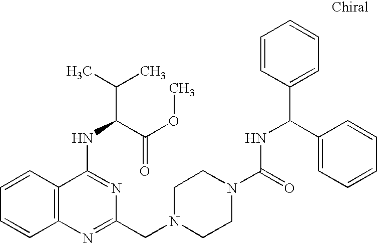 |
| 25-29 | 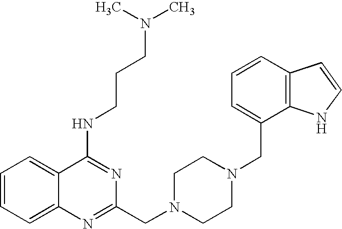 |
| 25-36 | 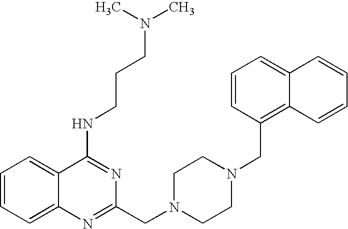 |
| 25-73 | 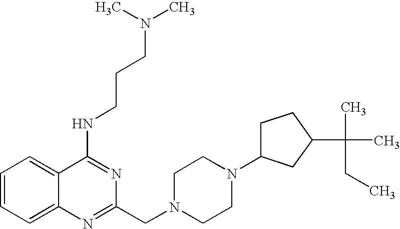 |
| 25-90 | 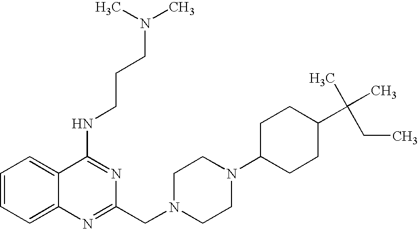 |
| 28-4 | 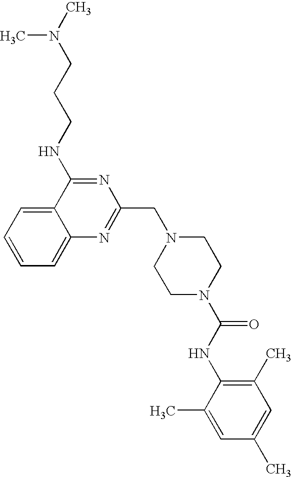 |
| 28-13 | 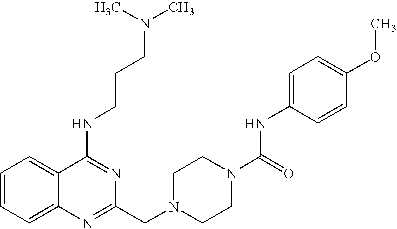 |
| 28-20 | 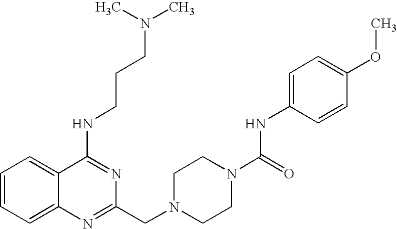 |
| 28-39 | 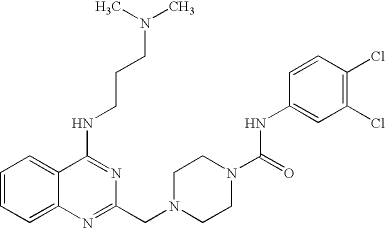 |
| 30-3 | 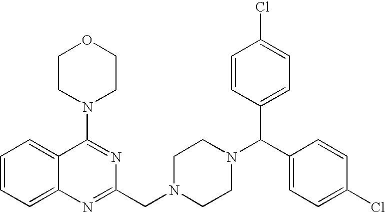 |
| 30-4 | 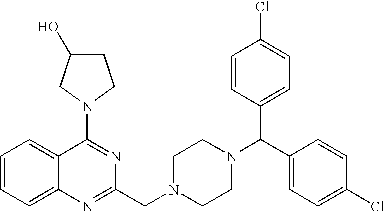 |
| 30-11 | 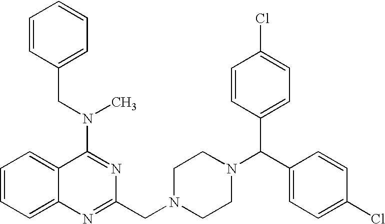 |
| 30-12 | 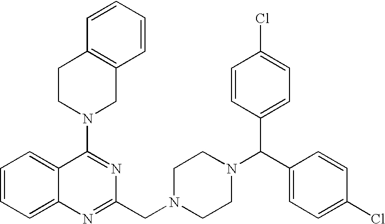 |
| 30-14 | 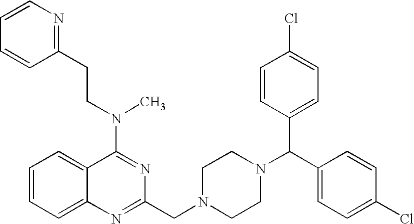 |
| 30-15 | 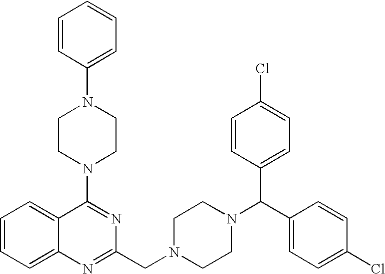 |
| 30-17 | 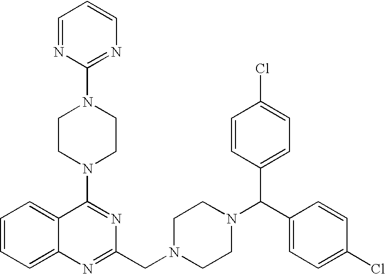 |
| 30-22 | 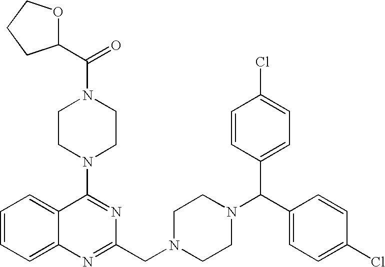 |
| 30-25 | 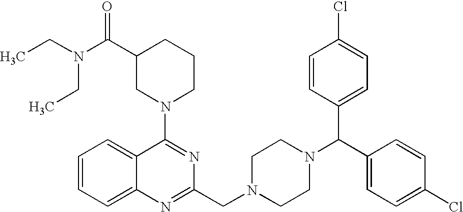 |
| 30-26 | 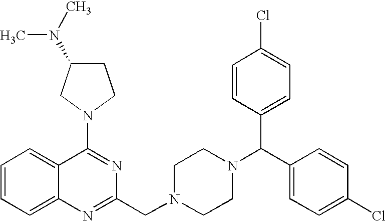 |
| 30-27 | 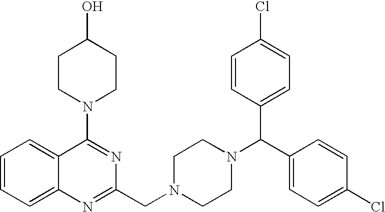 |
| 30-28 | 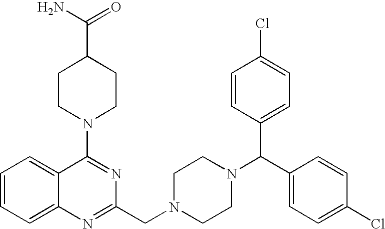 |
| 30-29 | 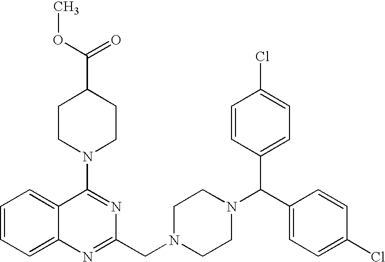 |
| 30-30 | 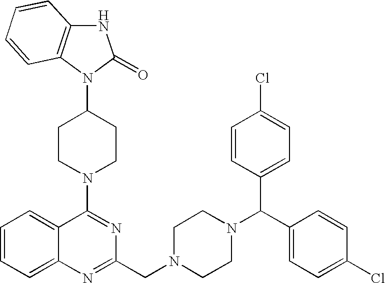 |
| 43 | 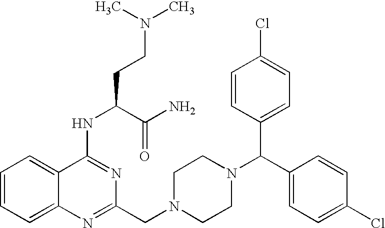 |
| 44 | 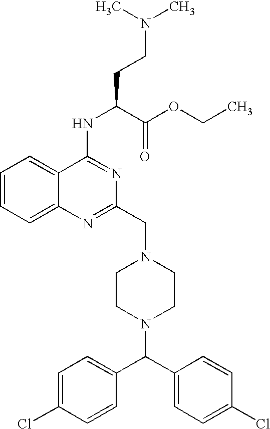 |
| 45 | 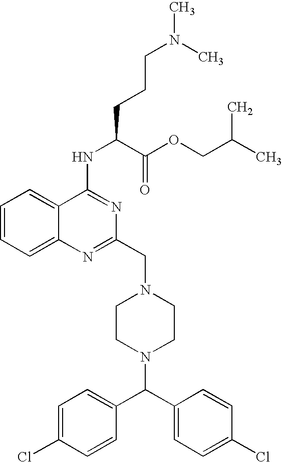 |
| 46 | 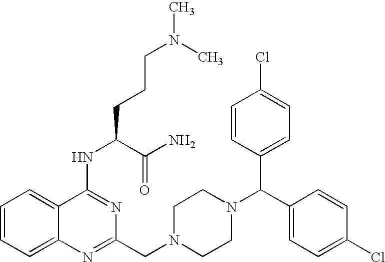 |
| 47 | 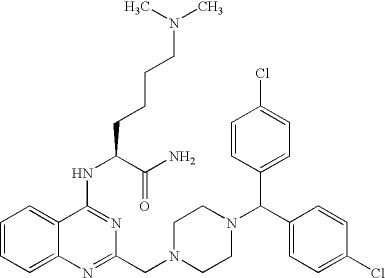 |

or a pharmaceutically acceptable salt, solvate or ester thereof.

42. The compound of claim 41, wherein said compound is selected from the group consisting of

| COMPOUND # | STRUCTURE |
|---|---|
| 1 | 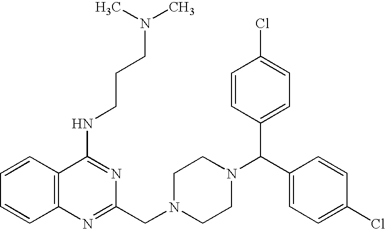 |
| 11-1 | 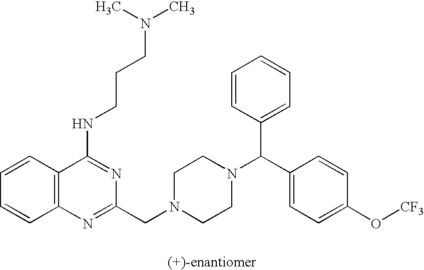 |
| 11-2 | 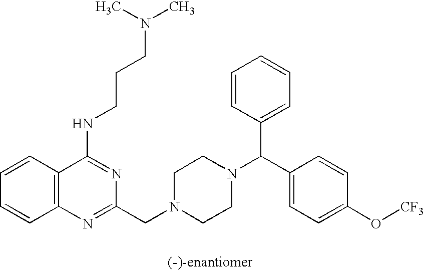 |
| 20-29 | 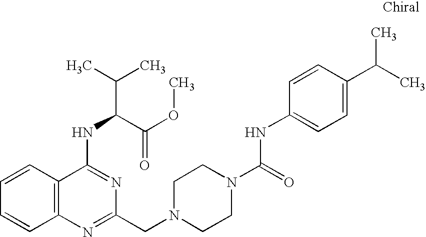 |
| 20-46 | 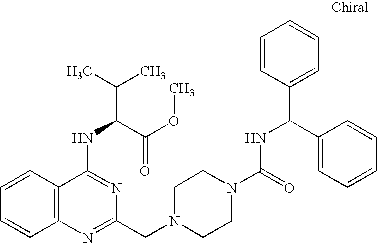 |
| 25-29 | 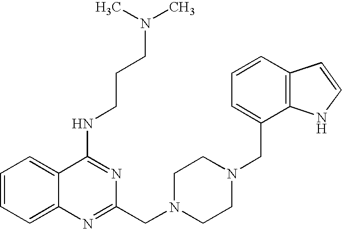 |
| 25-36 | 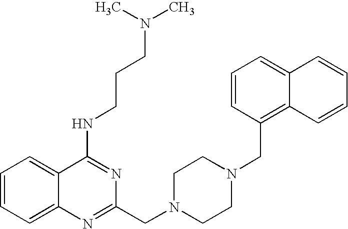 |
| 25-90 | 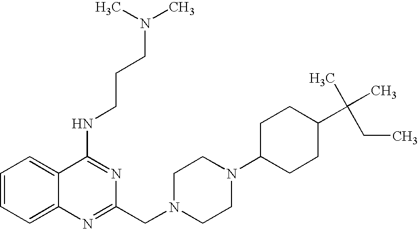 |
| 28-20 | 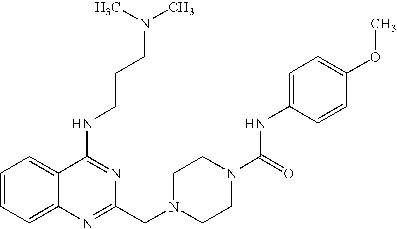 |
| 28-39 | 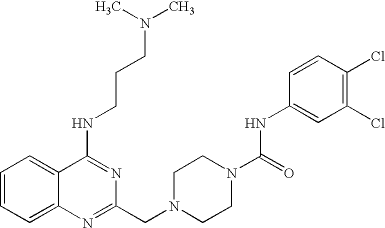 |
| 30-30 | 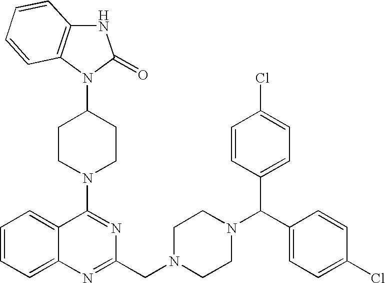 |
| 43 | 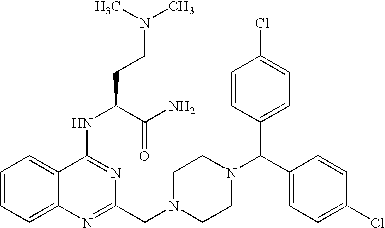 |
| 44 | 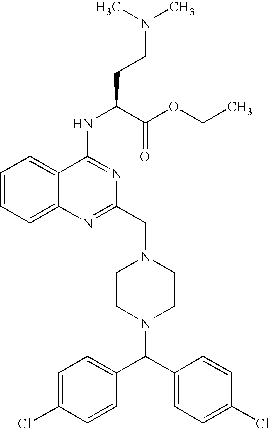 |
| 45 | 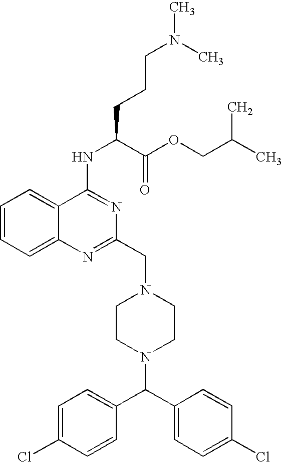 |
| 46 | 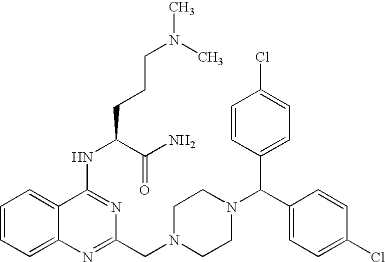 |
| 47 | 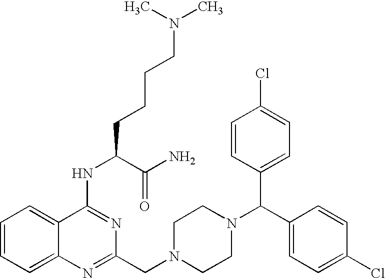 |

or a pharmaceutically acceptable salt, solvate or ester thereof.

43. The compound of claim 38, wherein said compound is selected from the group consisting of

| COMPOUND # | STRUCTURE |
|---|---|
| 1 | 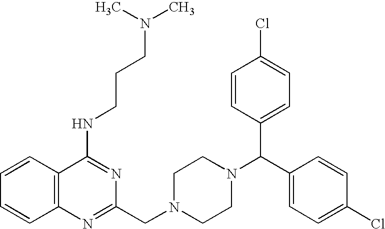 |
| 11-1 | 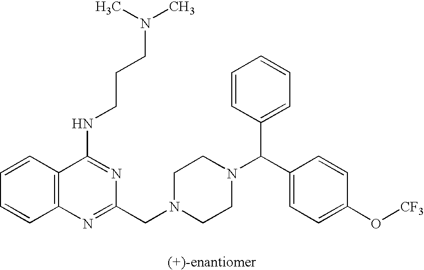 |
| 11-2 | 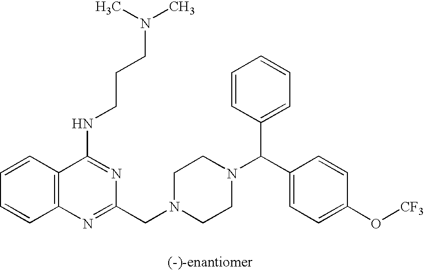 |
| 25-36 | 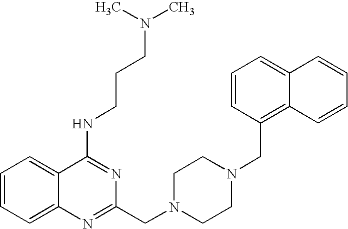 |
| 25-90 | 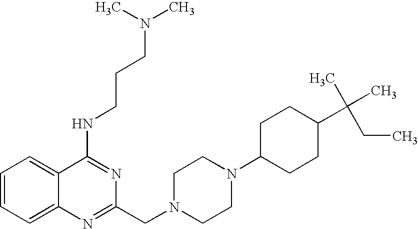 |

or a pharmaceutically acceptable salt, solvate or ester thereof.

44. The compound of claim 1, wherein X is OR⁵.

45. The compound of claim 44, wherein R¹ and R² are both hydrogen.

46. The compound of claim 45, wherein m is 0 or 1.

47. The compound of claim 46, wherein m is 0.

48. The compound of claim 46, wherein R⁵ is alkyl; wherein said alkyl is substituted with one moiety selected from the group consisting of heterocyclic, (alkyl)₂-amino, and alkoxy; wherein each of the alkyl alteratives of the aforesaid (alkyl)₂-amino and alkoxy moieties may optionally be substituted with an (alkyl)₂-amino radical.

49. The compound of claim 46, wherein R⁴ is alkyl; wherein said alkyl is substituted with two phenyl substituents; wherein each phenyl substituent is substituted with two halo moieties.

50. The compound of claim 1, selected from the group consisting of

| EXAMPLE | STRUCTURE |
|---|---|
| 31-1 | 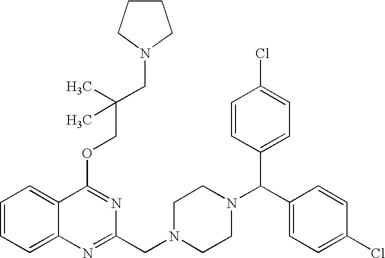 |
| 31-2 | 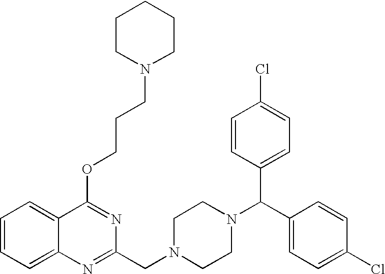 |
| 31-3 | 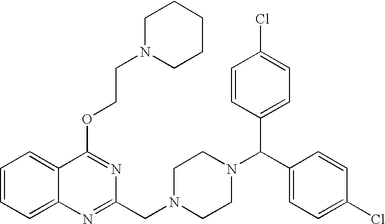 |
| 31-4 | 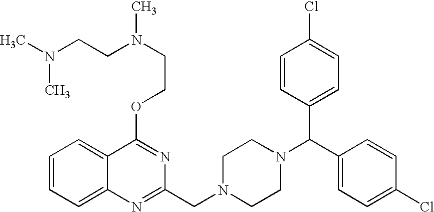 |
| 31-5 | 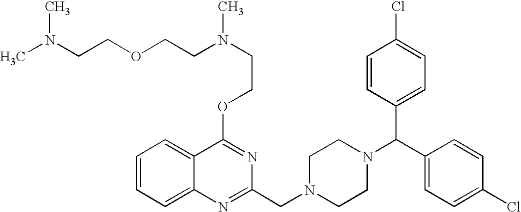 |
| 31-6 | 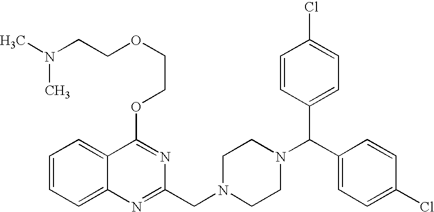 |

or a pharmaceutically acceptable salt, solvate or ester thereof.

51. An isolated or purified form of a compound of claim 1.

52. A pharmaceutical composition comprising a therapeutically effective amount of at least one compound of claim 1, or a pharmaceutically acceptable salt, solvate or ester thereof in combination with a pharmaceutically acceptable carrier.

53. The pharmaceutical composition of claim 52, further comprising one or more compounds selected from the group consisting of an anti-cancer agent, a PPAR-γ agonist, a PPAR-δ agonist, an inhibitor of inherent multidrug resistance, an anti-emetic agent, and an immunologic-enhancing drug.

54. The pharmaceutical composition of claim 53, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

55. The pharmaceutical composition of claim 54, wherein the cytotoxic agent is temozolamide.

56. A pharmaceutical composition comprising a therapeutically effective amount of a combination of at least one compound of claim 1 or a pharmaceutically acceptable salt, solvate or ester thereof, and temozolamide.

57. The pharmaceutical composition of claim 53, further comprising one or more anti-cancer agents selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

58. The pharmaceutical composition of claim 57, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

59. A method of treating a cellular proliferative disease in a subject comprising administering to said subject in need of such treatment a therapeutically effective amount of at least one compound of claim 1 or a pharmaceutically acceptable salt, solvate or ester thereof.

60. The method of claim 59, wherein the cellular proliferative disease is cancer, hyperplasia, cardiac hypertrophy, autoimmune diseases, fungal disorders, arthritis, graft rejection, inflammatory bowel disease, immune disorders, inflammation, cellular proliferation induced after medical procedures.

61. The method of claim 60, wherein the cancer is selected from cancers of the brain, genitourinary tract, cardiac, gastrointestine, liver, bone, nervous system, and lung.

62. The method of claim 60, wherein the cancer is selected from lung adenocarcinama, small cell lung cancer, pancreatic cancer, and breast carcinoma.

63. The method of claim 60, wherein said cancer is selected from pancreatic cancer and brain cancer.

64. The method of claim 60, further comprising radiation therapy.

65. The method of claim 59, further comprising administering to the subject at least one compound selected from the group consisting of an anti-cancer agent, a PPAR-γ agonist, a PPAR-δ agonist, an inhibitor of inherent multidrug resistance, an anti-emetic agent, and an immunologic-enhancing drug.

66. The method of claim 65, wherein the cellular proliferative disease is cancer.

67. The method of claim 66, further comprising radiation therapy.

68. The method of claim 65, wherein the anti-cancer agent is selected from the group consisting of an estrogen receptor modulator, an androgen receptor modulator, retinoid receptor modulator, a cytotoxic/cytostatic agent, an antiproliferative agent, a prenyl-protein transferase inhibitor, an HMG-CoA reductase inhibitor, an angiogenesis inhibitor, an inhibitor of cell proliferation and survival signaling, an agent that interferes with a cell cycle checkpoint, and an apoptosis inducing agent.

69. The method of claim 68, wherein the cytotoxic agent is temozolamide.

70. A method of treating a cellular proliferative disease in a subject comprising administering to said subject in need of such treatment a therapeutically effective amount of a combination of at least one compound of claim 1 or a pharmaceutically acceptable salt, solvate or ester thereof, and temozolamide.

71. The method of claim 65, further comprising one or more anti-cancer agent selected from the group consisting of cytostatic agent, cytotoxic agent, taxane, topoisomerase II inhibitor, topoisomerase I inhibitor, tubulin interacting agent, hormonal agent, thymidilate synthase inhibitor, anti-metabolite, alkylating agent, farnesyl protein transferase inhibitor, signal transduction inhibitor, EGFR kinase inhibitor, antibody to EGFR, C-abl kinase inhibitor, hormonal therapy combination, and aromatase combination.

72. The method of claim 65, further comprising one or more agents selected from the group consisting of Uracil mustard, Chlormethine, Ifosfamide, Melphalan, Chlorambucil, Pipobroman, Triethylenemelamine, Triethylenethiophosphoramine, Busulfan, Carmustine, Lomustine, Streptozocin, Dacarbazine, Floxuridine, Cytarabine, 6-Mercaptopurine, 6-Thioguanine, Fludarabine phosphate, oxaliplatin, leucovirin, oxaliplatin, Pentostatine, Vinblastine, Vincristine, Vindesine, Bleomycin, Dactinomycin, Daunorubicin, Doxorubicin, Epirubicin, Idarubicin, Mithramycin, Deoxycoformycin, Mitomycin-C, L-Asparaginase, Teniposide 17α-Ethinylestradiol, Diethylstilbestrol, Testosterone, Prednisone, Fluoxymesterone, Dromostanolone propionate, Testolactone, Megestrolacetate, Methylprednisolone, Methyltestosterone, Prednisolone, Triamcinolone, Chlorotrianisene, Hydroxyprogesterone, Aminoglutethimide, Estramustine, Medroxyprogesteroneacetate, Leuprolide, Flutamide, Toremifene, goserelin, Cisplatin, Carboplatin, Hydroxyurea, Amsacrine, Procarbazine, Mitotane, Mitoxantrone, Levamisole, Navelbene, Anastrazole, Letrazole, Capecitabine, Reloxafine, Droloxafine, Hexamethylmelamine, doxorubicin, cyclophosphamide, gemcitabine, interferons, pegylated interferons, Erbitux and mixtures thereof.

73. A process for potentiating the growth suppression activity of temolozomide in cancer cells comprising administering to said cells a therapeutically effective amount of a combination of at least one compound of claim 1 or a pharmaceutically acceptable salt, solvate or ester thereof, and temozolamide.

74. The process of claim 73, wherein said cancer cells are selected from the group consisting of pancreatic cells and glioma cells.
